(19) United States
(12) Patent Application Publication
(10) Pub. No.: US 2024/0254119 A1
(43) Pub. Date: Aug. 1, 2024

(54) COMPOUNDS AND COMPOSITIONS AS c-Kit KINASE INHIBITORS

(71) Applicant: THIRD HARMONIC BIO, INC., San Francisco, CA (US)

(72) Inventors: Gregg F. KEANEY, Lexington, MA (US); Brian T. CHAMBERLAIN, Cape Elizabeth, ME (US); Caitlin Nicole KENT, Waltham, MA (US); John A. LOWE, III, Stonington, CT (US); Matthew Charles LUCAS, Lexington, MA (US)

(21) Appl. No.: 18/532,573

(22) Filed: Dec. 7, 2023

Related U.S. Application Data
(60) Provisional application No. 63/515,031, filed on Jul. 21, 2023.
(60) Provisional application No. 63/430,918, filed on Dec. 7, 2022.

(51) Int. Cl. C07D 471/04; C07D 487/04; C07D 491/147

(52) U.S. Cl. CPC C07D 471/04; C07D 487/04; C07D 491/147

(57) ABSTRACT

The invention provides novel compounds, pharmaceutical compositions, and their use for inhibiting c-kit kinase activity.

### CROSS REFERENCE TO RELATED APPLICATIONS

This application claims the benefit of and priority to U.S. Provisional Patent Application Ser. No. 63/430,918, filed Dec. 7, 2022, and U.S. Provisional Patent Application Ser. No. 63/515,031, filed Jul. 21, 2023; the contents of which are hereby incorporated by reference.

### FIELD OF THE INVENTION

The present disclosure relates generally to various compounds and compositions useful as a selective inhibitor of c-kit kinase and uses of the same in the treatment of c-kit kinase associated diseases, disorders, and conditions.

### BACKGROUND

Compounds of the present disclosure are selective inhibitors of c-kit kinase, useful for the depletion of mast cells and thus is useful for treating mast-cell associated diseases including, inter alia, asthma, allergic rhinitis, pulmonary arterial hypertension (PAH), primary pulmonary hypertension (PPH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, food allergy, chronic rhinosinusitis, type I diabetes, type II diabetes, systemic sclerosis, allergic keratoconjunctivitis, vernal keratoconjunctivitis, Crohn's disease, or systemic and cutaneous lupus erythematosus and dermatomyositis. Exemplary other mast-cell diseases are described further herein.

There remains a need in the art for novel compounds, compositions and methods for treating mast-cell associated diseases.

### SUMMARY

The present disclosure provides compounds, compositions, and methods of treating c-kit kinase mediated diseases comprising administering to a patient in need thereof a compound of the present disclosure, or a pharmaceutical salt or composition thereof. In general, the compounds and methods disclosed herein are useful for treating mast-cell associated diseases as described herein.

In some embodiments, a compound of the present disclosure is represented by Formula I:

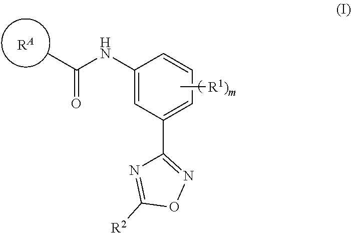

or a pharmaceutically acceptable salt thereof, where the variables are as defined in the detailed description.

In some embodiments, a compound of the present disclosure is represented by Formula I-1:

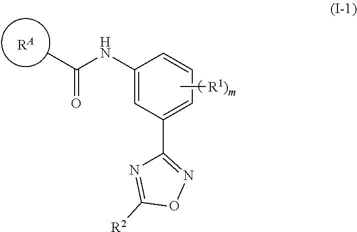

or a pharmaceutically acceptable salt thereof, where the variables are as defined in the detailed description. Further description of such compounds is described herein in the detailed description. The compounds may be part of a pharmaceutical composition comprising a pharmaceutically acceptable carrier.

In some embodiments, the invention provides a method of treating a disorder mediated by c-kit kinase in a subject. The method comprises administering a therapeutically effective amount of a compound described herein, such as a compound of Formula I, to a subject in need thereof to treat the disorder, as further described in the detailed description. Further methods comprise administering a therapeutically effective amount of a compound described herein, such as a compound of Formula I-1, to a subject in need thereof to treat the disorder, as further described in the detailed description.

In some embodiments, the invention provides methods of inhibiting c-kit kinase activity. Some such methods comprise contacting c-kit kinase with an effective amount of a compound described herein, such as a compound of Formula I, to inhibit c-kit kinase activity, as further described in the detailed description. Further methods comprise contacting c-kit kinase with an effective amount of a compound described herein, such as a compound of Formula I-1, to inhibit c-kit kinase activity, as further described in the detailed description.

### DETAILED DESCRIPTION

The present disclosure is based at least in part on the identification of novel compounds that modulate c-kit kinase and methods of using the same to treat c-kit kinase associated diseases. Disclosed herein are compounds of Formula I:

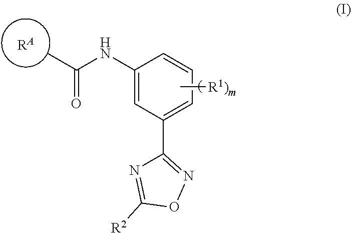

or a pharmaceutically acceptable salt thereof, wherein:
R¹ represents independently for each occurrence halogen, —CN, C₁₋₆ alkyl, or C₁₋₆ haloalkyl;
R² is C₁₋₆ aliphatic, phenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; hydrogen; or L¹-R⁴, wherein R² is substituted with p occurrences of R⁶;
L¹ is a C₁₋₃ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R⁴ is a 3-7 membered saturated or partially unsaturated monocyclic carbocyclic ring, a 3-7 membered saturated or partially unsaturated monocyclic heterocyclic ring having 1-2 heteroatoms independently selected from nitrogen, oxygen, and sulfur, or optionally substituted phenyl;
R⁶ represents independently for each occurrence oxo, halogen, C₁₋₆ aliphatic, C₁₋₆ haloaliphatic, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or optionally substituted phenyl;
R^{A} is of any of the following structures:

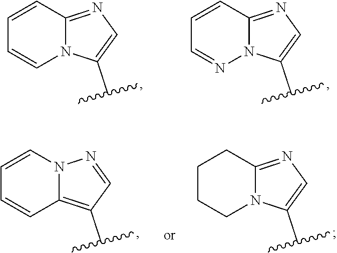 [SMILES: *c1cnc2ccccn12 ; *c1cnc2cccnn12 ; *c1cnn2ccccc12 ; *c1cnc2n1CCCC2]

each of which is substituted by n occurrences of R³;
R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(R)₂OCR₃, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, -L²-R⁵, or an optionally substituted group selected from C₁₋₆ aliphatic, C₁₋₆ haloaliphatic, phenyl, naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; each of which is substituted with r instances of R; or:
two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur, substituted with r instances of R;
L² represents independently for each occurrence a C₁₋₆ bivalent saturated or unsaturated, straight or branched hydrocarbon chain wherein one, two, or three methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂—or -Cy-;
Cy represents independently for each occurrence phenyl, a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
R⁵ represents independently for each occurrence hydrogen, OR, C₁₋₆ aliphatic, C₁₋₆ haloaliphatic, or phenyl fused to a 5-6 membered saturated heterocyclic ring having 1-2 heteroatoms independently selected from nitrogen, oxygen, and sulfur;
each R is independently hydrogen, —CN, halogen, oxo, or an optionally substituted group selected from C₁₋₆ aliphatic; C₁₋₆ haloaliphatic; C₁₋₃ hydroxyalkyl; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur;
m is 0 or 1;
n is 0, 1, 2, 3, 4, or 5;
p is 0, 1, 2, 3, 4, or 5; and
r is 0, 1, 2, 3, 4, or 5.

Additionally disclosed herein are compounds of Formula I-1:

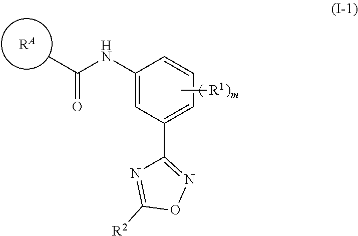

or a pharmaceutically acceptable salt thereof, wherein:
R¹ represents independently for each occurrence halogen, —CN, —OR, C₁₋₆ alkyl, or C₁₋₆ haloalkyl;
R² is C₁₋₆ aliphatic, phenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; or L¹-R⁴, wherein R² is substituted with p occurrences of R⁶;
L¹ is a C₁₋₂ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R⁴ is a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur;
R⁶ represents independently for each occurrence oxo, halogen, C₁₋₆ aliphatic, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or optionally substituted phenyl;
R^{A} is of any of the following structures:

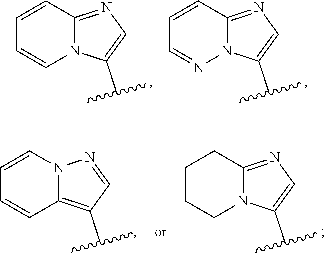

each of which is substituted by n occurrences of R³;
R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, -L²-R⁵, or an optionally substituted group selected from C₁₋₆ aliphatic, C₁₋₆ haloaliphatic, phenyl, naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; each of which is substituted with r instances of R; or:
two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur;
L² represents independently for each occurrence a C₁₋₆ bivalent saturated or unsaturated, straight or branched hydrocarbon chain wherein one, two, or three methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂— or -Cy-;
Cy represents independently for each occurrence phenyl, a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
R⁵ represents independently for each occurrence hydrogen, OR, C₁₋₆ aliphatic, C₁₋₆ haloaliphatic, or phenyl fused to a 5-6 membered saturated heterocyclic ring having 1-2 heteroatoms independently selected from nitrogen, oxygen, and sulfur;
each R is independently hydrogen, —CN, halogen, or an optionally substituted group selected from C₁₋₆ aliphatic; C₁₋₆ haloaliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur;
m is 0, 1, 2, 3, or 4;
n is 0, 1, 2, 3, 4, or 5;
p is 0, 1, 2, 3, 4, or 5; and
r is 0, 1, 2, 3, 4, or 5.

The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

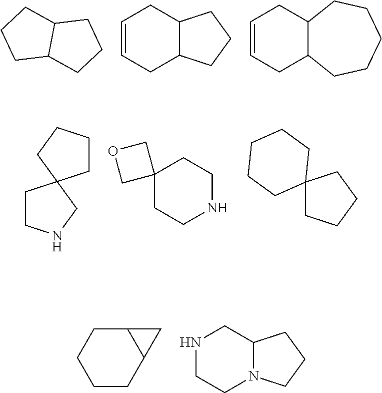

Exemplary bridged bicyclics include:

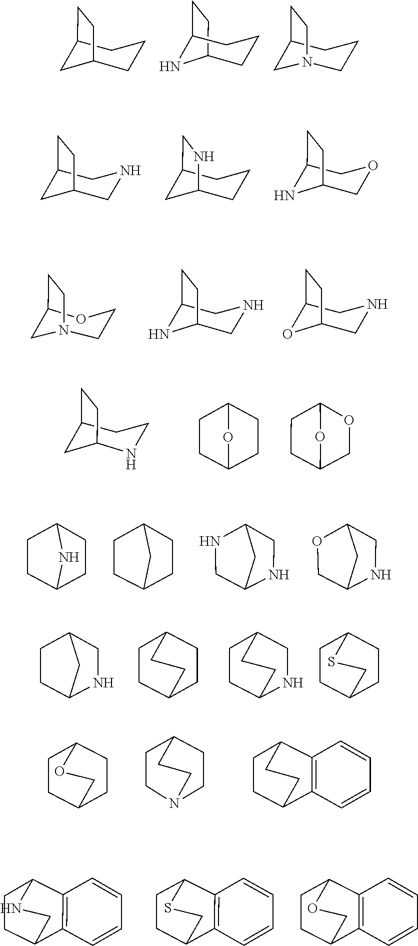

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

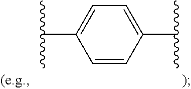 [SMILES: *c1ccc(*)cc1]

“phenylene” is a trivalent phenyl group when it has three groups attached to it

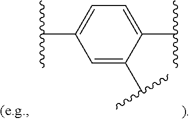 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it. The term “pyridinylene” refers to a multivalent pyridine radical having the appropriate number of open valences to account for groups attached to it. For example, “pyridinylene” is a bivalent pyridine radical when it has two groups attached to it

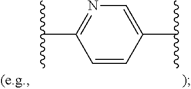 [SMILES: *c1ccc(*)nc1]

“pyridinylene” is a trivalent pyridine radical when it has three groups attached to it

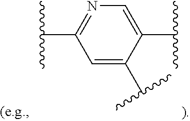 [SMILES: *c1cc(*)c(*)cn1]

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by an oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄0S(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}*.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*2))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when RT is C₁₋₆ aliphatic, R^{†} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66 (1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomers are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The term “carbocyclylene” refers to a multivalent carbocyclyl group having the appropriate number of open valences to account for groups attached to it. For example, “carbocyclylene” is a bivalent carbocyclyl group when it has two groups attached to it; “carbocyclylene” is a trivalent carbocyclyl group when it has three groups attached to it.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like. The term “hydroxyalkoxyl” refers to an alkoxyl group that is substituted with at least one hydroxyl. Exemplary hydroxyalkoxyl groups include —OCH₂CH₂OH, —OCH₂C(H)(OH)CH₂CH₂OH, and the like. The term “alkoxylene” refers to a bivalent alkoxyl group.

The term “oxo” is art-recognized and refers to a “═O” substituent. For example, a cyclopentane substituted with an oxo group is cyclopentanone.

The symbol “ [symbol] ” indicates a point of attachment.

When a chemical structure containing a ring is depicted with a substituent having a bond that crosses a ring bond, the substituent may be attached at any available position on the ring. For example, the chemical structure

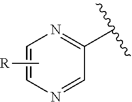

encompasses

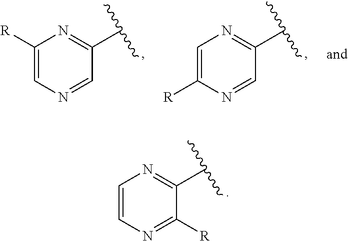

In the context of a polycyclic fused ring, when a chemical structure containing a polycyclic fused ring is depicted with one or more substituent(s) having a bond that crosses multiple rings, the one or more substituent(s) may be independently attached to any of the rings crossed by the bond. To illustrate, the chemical structure

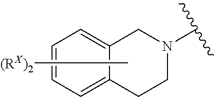

encompasses, for example,

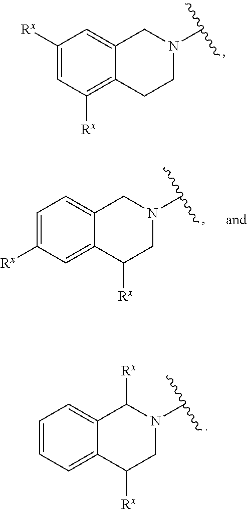

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and, most preferably, includes humans.

The term “IC₅₀” is art-recognized and refers to the concentration of a compound that is required to achieve 50% inhibition of the target. The potency of an inhibitor is usually defined by its IC₅₀ value. The lower the IC₅₀ value the greater the potency of the antagonist and the lower the concentration that is required to inhibit the maximum biological response. In certain embodiments, an inhibitor has an IC₅₀ and/or binding constant of less than about 100 μM, less than about 50 μM, less than about 1 μM, less than about 500 nM, less than about 100 nM, less than about 10 nM, or less than about 1 nM.

As used herein, the terms “inhibitor” or “c-kit inhibitor” are defined as a compound that binds to and/or inhibits c-kit kinase with measurable affinity. In some embodiments, inhibition in the presence of the inhibitor is observed in a dose-dependent manner. In some embodiments, the measured signal (e.g., signaling activity or biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100% lower than the signal measured with a negative control under comparable conditions.

The terms “measurable affinity” and “measurably inhibit,” as used herein, means a measurable change or inhibition in c-kit kinase activity between a sample comprising a compound of the present invention, or composition thereof an equivalent sample comprising c-kit kinase, in the absence of said compound, or composition thereof.

As used herein, the term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example, to prevent or delay their recurrence.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Compounds of the Present Disclosure

The present disclosure provides compounds and pharmaceutically acceptable salts thereof that may be used in pharmaceutical compositions and therapeutic methods described herein. Exemplary compounds are described in the following sections, along with exemplary procedures for making the compounds.

### Formula (I)

In some embodiments, the present disclosure provides a compound represented by Formula (I):

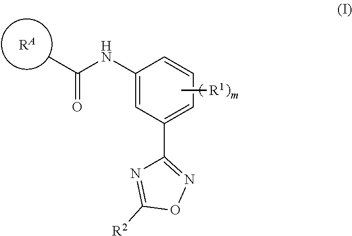

or a pharmaceutically acceptable salt thereof, wherein:
R¹ represents independently for each occurrence halogen, —CN, C₁₋₆ alkyl, or C₁₋₆ haloalkyl;
R² is C₁₋₆ aliphatic, phenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; hydrogen; or L¹-R⁴, wherein R² is substituted with p occurrences of R⁶;
L¹ is a C₁₋₃ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R⁴ is a 3-7 membered saturated or partially unsaturated monocyclic carbocyclic ring, a 3-7 membered saturated or partially unsaturated monocyclic heterocyclic ring having 1-2 heteroatoms independently selected from nitrogen, oxygen, and sulfur, or optionally substituted phenyl;
R⁶ represents independently for each occurrence oxo, halogen, C₁₋₆ aliphatic, C₁₋₆ haloaliphatic, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or optionally substituted phenyl;
R^{A} is of any of the following structures:

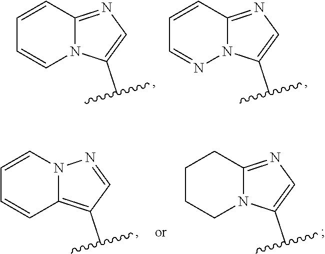

each of which is substituted by n occurrences of R³;
R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(R)₂OCR₃, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, -L²-R⁵, or an optionally substituted group selected from C₁₋₆ aliphatic, C₁₋₆ haloaliphatic, phenyl, naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; each of which is substituted with r instances of R; or:
two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur, substituted with r instances of R;
L² represents independently for each occurrence a C₁₋₆ bivalent saturated or unsaturated, straight or branched hydrocarbon chain wherein one, two, or three methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂— or -Cy-;
Cy represents independently for each occurrence phenyl, a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
R⁵ represents independently for each occurrence hydrogen, OR, C₁₋₆ aliphatic, C₁₋₆ haloaliphatic, or phenyl fused to a 5-6 membered saturated heterocyclic ring having 1-2 heteroatoms independently selected from nitrogen, oxygen, and sulfur;
each R is independently hydrogen, —CN, halogen, oxo, or an optionally substituted group selected from C₁₋₆ aliphatic; C₁₋₆ haloaliphatic; C₁₋₃ hydroxyalkyl; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur;
m is 0 or 1;
n is 0, 1, 2, 3, 4, or 5;
p is 0, 1, 2, 3, 4, or 5; and
r is 0, 1, 2, 3, 4, or 5.

The definitions of variables in Formula I above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

The description above describes multiple embodiments relating to compounds of Formula I. The patent application specifically contemplates all combinations of the embodiments.

It has been surprisingly discovered that certain compounds of the present invention do not significantly penetrate the brain or minimally penetrate the brain, wherein the extent of brain penetration is measured by measuring “Kₚ,” i.e., the ratio of compound concentration in the brain and blood plasma (C_{brain}/Cₚₗₐₛₘₐ) as demonstrated by certain assays described herein. Exemplary such compounds include, e.g., I-296. In some such embodiments, a compound of the present invention is characterized as having a Kₚ (brain) of less than about 0.7, less than about 0.6, less than about 0.5, less than about 0.4, less than about 0.3, less than about 0.2, or less than about 0.1. In some embodiments, a compound of the present invention is characterized as having a Kₚ of less than about 0.7. In some embodiments, a compound of the present invention is characterized by having a Kₚ of less than about 0.6. In some embodiments, a compound of the present invention is characterized by having a Kₚ of less than about 0.5. In some embodiments, a compound of the present invention is characterized by having a Kₚ of less than about 0.4. In some embodiments, a compound of the present invention is characterized by having a Kₚ of less than about 0.3. In some embodiments, a compound of the present invention is characterized by having a Kₚ of less than about 0.2. In some embodiments, a compound of the present invention is characterized by having a Kₚ of less than about 0.1. In some embodiments, a compound of the present invention is characterized by having a Kₚ of less than about 0.09. In some embodiments, a compound of the present invention is characterized by having a Kₚ of less than about 0.08. In some embodiments, a compound of the present invention is characterized by having a Kₚ of less than about 0.05. In some embodiments, a compound of the present invention is characterized by having a Kₚ of less than about 0.04. In some embodiments, a compound of the present invention is characterized by having a Kₚ of less than about 0.03. In some embodiments, a compound of the present invention is characterized by having a Kₚ of less than about 0.02. In some embodiments, a compound of the present invention is characterized by having a Kₚ of less than about 0.01. Various methods of assessing brain exposure are known to those of skill in the art and/or are described herein. Exemplary such compounds which do not significantly penetrate the brain or minimally penetrate the brain, as indicated by Kₚ, include, but are not limited to, I-114, I-71, I-84, I-114, and I-296.

In some embodiments, for compounds with low Kₚ values, determination of Kₚᵤᵤ from the unbound compounds plasma, brain and testes concentrations also supports peripheral restriction of the compounds.

In some embodiments, it has been surprisingly discovered that compounds of the present invention are Breast Cancer Resistance Protein (BCRP) efflux substrates. The human breast cancer resistance protein (BCRP, gene symbol ABCG2) is an ATP-binding cassette (ABC) efflux transporter. Among normal human tissues, BCRP is highly expressed on the apical membranes of the placental syncytiotrophoblasts, the intestinal epithelium, the liver hepatocytes, the endothelial cells of brain microvessels, testis, and the renal proximal tubular cells, contributing to the absorption, distribution, and elimination of drugs and endogenous compounds as well as tissue protection against xenobiotic exposure. As a result, BCRP has now been recognized by the FDA to be one of the key drug transporters involved in clinically relevant drug disposition.

Various methods of assessing whether a compound is a BCRP efflux substrate are known to those of skill in the art and/or are described herein, for instance in Example 99. Exemplary such compounds include, e.g., I-296. In some embodiments, a compound of the present invention exhibits an efflux ratio (ER) of about 1-fold, indicating substantially no efflux. In some embodiments, a compound of the present invention exhibits an efflux ratio (ER) of at least about 1.5-fold. In some embodiments, a compound of the present invention exhibits an efflux ratio (ER) of at least about 2.0-fold. In some embodiments, a compound of the present invention exhibits an efflux ratio (ER) of at least about 3.5-fold. In some embodiments, a compound of the present invention exhibits an efflux ratio (ER) of at least about 4.0-fold. In some embodiments, a compound of the present invention exhibits an efflux ratio (ER) of at least about 4.5-fold. In some embodiments, a compound of the present invention exhibits an efflux ratio (ER) of at least about 5-fold. In some embodiments, a compound of the present invention exhibits an efflux ratio (ER) of at least about 6-fold. In some embodiments, a compound of the present invention exhibits an efflux ratio (ER) of at least about 7-fold. In some embodiments, a compound of the present invention exhibits an efflux ratio (ER) of at least about 8-fold. In some embodiments, a compound of the present invention exhibits an efflux ratio (ER) of at least about 9-fold. In some embodiments, a compound of the present invention exhibits an efflux ratio (ER) of at least about 10-fold. In some embodiments, a compound of the present invention exhibits an efflux ratio (ER) of at least about 15-fold. In some embodiments, a compound of the present invention exhibits an efflux ratio (ER) of at least about 20-fold. In some embodiments, a compound of the present invention exhibits an efflux ratio (ER) of at least about 25-fold. In some embodiments, a compound of the present invention exhibits an efflux ratio (ER) of at least about 30-fold. In some embodiments, a compound of the present invention exhibits an efflux ratio (ER) of at least about 35-fold. In some embodiments, a compound of the present invention exhibits an efflux ratio (ER) of at least about 40-fold. In some embodiments, a compound of the present invention exhibits an efflux ratio (ER) of at least about 45-fold. In some embodiments, a compound of the present invention exhibits an efflux ratio (ER) of at least about 50-fold.

In some embodiments, it has been surprisingly discovered that certain compounds of the present invention do not significantly inhibit BCRP.

Various methods of assessing whether a compound is a BCRP inhibitor are known to those of skill in the art and/or are described herein, for instance in Example 103. Exemplary such compounds include, e.g., I-296. In some embodiments, compounds of the present invention have a BCRP inhibition IC₅₀ of about 400 nM, or about 500 nM, or about 600 nM, or about 700 nM, or about 800 nM, or about 900 nM. In some embodiments, compounds of the present invention have a BCRP inhibition IC₅₀ of about 1 mM, about 2 mM, about 3 mM, about 4 mM, about 5 mM, about 6 mM, about 7 mM, about 8 mM, about 9 mM, or about 10 mM. In some embodiments, compounds of the present invention have a BCRP inhibition IC₅₀ of between about 500 nM and 10 mM. In some embodiments, compounds of the present invention have a BCRP inhibition IC₅₀ of between about 500 nM and 5 mM. In some embodiments, compounds of the present invention have a BCRP inhibition IC₅₀ of between about 500 nM and 1 mM. In some embodiments, compounds of the present invention have a BCRP inhibition IC₅₀ of between about 1 mM and 10 mM. In some embodiments, compounds of the present invention have a BCRP inhibition IC₅₀ of between about 1 mM and 5 mM. In some embodiments, compounds of the present invention have a BCRP inhibition IC₅₀ of between about 5 mM and 10 mM. Exemplary compounds of the present invention are described further herein.

In some embodiments, it has been surprising discovered that compounds of the present invention are P-glycoprotein (PGP) efflux substrates. P-glycoprotein (PGP), an efflux membrane transporter, is also referred to in the art as multi-drug resistance protein 1 ((MDR1), permeability glycoprotein, P-gp, or Pgp, encoded by MDR1/ABCB1 and belonging to the family of ATP-binding cassette transporters), and is widely distributed throughout the body and responsible for limiting cellular uptake and the distribution of xenobiotics and toxic substances. PGP is one of the most important transporters at the blood-brain barrier (BBB), where it is highly expressed in the vessel walls of the brain capillaries functioning as an efflux pump. PGP is also located throughout the human body in organs or tissues with an excretory and/or barrier function, such as the liver, kidney, placenta, and testes.

With respect to the placenta, PGP has been found to have a role in the regulation of drug disposition to the fetus and has been extensively studied. Expression of PGP in the placental trophoblast layer has been confirmed at the mRNA and protein levels in all phases of pregnancy. Several in vitro and in vivo studies have demonstrated functional activity of the transporter in materno-fetal drug transport. PGP is able to actively pump drugs and other xenobiotics from trophoblast cells back to the maternal circulation, thus providing protection to the fetus.

In some embodiments, compounds of the present invention are efflux substrates of BRCP. In some embodiments, compounds of the present invention are efflux substrates of PGP. In some embodiments, compounds of the present invention are efflux substrates of one or both of BCRP and PGP.

It has been further surprisingly discovered that certain compounds of the present invention afford lower testes exposure, which may lead to better spermatogonia survival and/or spermatogonia maturation. Various methods of assessing whether a compound affords lower testes exposure are known to those of skill in the art and/or are described herein, for instance in Example 8. Exemplary such compounds include, e.g., I-296. Kₚ (testes) is defined as the ratio of compound concentration in the testes and in the plasma (Cₜₑₛₜₑₛ/Cₚₗₐₛₘₐ). By lower testes exposure is meant a compound measured as having a Kₚ (testes) of less than about 1.0, or less than about 0.9, or less than about 0.8, or less than about 0.7, or less than about 0.6, or less than about 0.5, or less than about 0.4, or less than about 0.3, or less than about 0.2, or less than about 0.1, or less than about 0.09, or less than about 0.08, or less than about 0.07, or less than about 0.06, or less than about 0.05, or less than about 0.04, or 0.03, or less than about 0.02, or less than about 0.01.

In some embodiments, a compound of the present invention is not an inducer of CYP3A4, as measured by CYP3A4 gene expression, for instance, see Example 101. In some embodiments, such may lead to reduced risk for drug-drug interactions. Exemplary such compounds include, e.g., I-296. For instance, in some embodiments, a compound of the present invention when measured as described herein exhibits an induction fold of less than or equal to about 10-fold. In some embodiments, a compound of the present invention when measured as described herein exhibits an induction fold of less than or equal to about 9-fold. In some embodiments, a compound of the present invention when measured as described herein exhibits an induction fold of less than or equal to about 8-fold. In some embodiments, a compound of the present invention when measured as described herein exhibits an induction fold of less than or equal to about 7-fold. In some embodiments, a compound of the present invention when measured as described herein exhibits an induction fold of less than or equal to about 6-fold. In some embodiments, a compound of the present invention when measured as described herein exhibits an induction fold of less than or equal to about 5-fold. In some embodiments, a compound of the present invention when measured as described herein exhibits an induction fold of less than or equal to about 4-fold. In some embodiments, a compound of the present invention when measured as described herein exhibits an induction fold of less than or equal to about 3-fold. In some embodiments, a compound of the present invention when measured as described herein exhibits an induction fold of less than or equal to about 2-fold.

In some embodiments, a compound of the present invention is not an inducer of CYP1A2, as measured by CYP1A2 gene expression, for instance, in Example 6. In some embodiments, such may lead to reduced risk for drug-drug interactions. Exemplary such compounds include, e.g., I-296. For instance, in some embodiments, a compound of the present invention when measured as described herein exhibits an induction fold of less than or equal to about 10-fold. In some embodiments, a compound of the present invention when measured as described herein exhibits an induction fold of less than or equal to about 9-fold. In some embodiments, a compound of the present invention when measured as described herein exhibits an induction fold of less than or equal to about 8-fold. In some embodiments, a compound of the present invention when measured as described herein exhibits an induction fold of less than or equal to about 7-fold. In some embodiments, a compound of the present invention when measured as described herein exhibits an induction fold of less than or equal to about 6-fold. In some embodiments, a compound of the present invention when measured as described herein exhibits an induction fold of less than or equal to about 5-fold. In some embodiments, a compound of the present invention when measured as described herein exhibits an induction fold of less than or equal to about 4-fold. In some embodiments, a compound of the present invention when measured as described herein exhibits an induction fold of less than or equal to about 3-fold. In some embodiments, a compound of the present invention when measured as described herein exhibits an induction fold of less than or equal to about 2-fold.

In some embodiments, a compound of the present invention is not an inducer of CYP2C19, as measured by CYP2C19 gene expression, which may lead to reduced risk for drug-drug interactions. For instance, in some embodiments, a compound of the present invention when measured as described herein exhibits an induction fold of less than or equal to about 10-fold. In some embodiments, a compound of the present invention when measured as described herein exhibits an induction fold of less than or equal to about 9-fold. In some embodiments, a compound of the present invention when measured as described herein exhibits an induction fold of less than or equal to about 8-fold. In some embodiments, a compound of the present invention when measured as described herein exhibits an induction fold of less than or equal to about 7-fold. In some embodiments, a compound of the present invention when measured as described herein exhibits an induction fold of less than or equal to about 6-fold. In some embodiments, a compound of the present invention when measured as described herein exhibits an induction fold of less than or equal to about 5-fold. In some embodiments, a compound of the present invention when measured as described herein exhibits an induction fold of less than or equal to about 4-fold. In some embodiments, a compound of the present invention when measured as described herein exhibits an induction fold of less than or equal to about 3-fold. In some embodiments, a compound of the present invention when measured as described herein exhibits an induction fold of less than or equal to about 2-fold.

In some embodiments, a compound of Formula I is administered orally, as described further herein. In some embodiments, a compound of Formula I is administered by a means other than oral administration, as described further herein.

In some embodiments, it has been unexpectedly found that certain compounds of Formula I exhibit improved solubility as compared to c-KIT inhibitors known in the art when measured according to the procedure set forth in Example 102 herein. In some embodiments, compounds of the present invention have a solubility greater than 2.0 μM and less than or equal to 10.0 μM. In some embodiments, compounds of the present invention have a solubility of about 2.5 μM, about 3.0 μM, about 3.5 μM, about 4.0 μM, about 4.5 μM, about 5.0 μM, about 5.5 μM, about 6.0 μM, about 6.5 μM, about 7.0 μM, about 7.5 μM, about 8.0 μM, about 9.0 μM, about 9.5 μM, or about 10.0 μM. In some embodiments, compounds of the present invention have a solubility greater than 10 μM and less than or equal to 50 μM. In some embodiments, compounds of the present invention have a solubility of about 15 μM, about 20 μM, about 25 μM, about 30 μM, about 35 μM, about 40 μM, about 45 μM, or about 50 μM. In some embodiments, compounds of the present invention have a solubility greater than 50 μM. In some embodiments, compounds of the present invention have a solubility of about 60 μM, about 70 μM, about 80 μM, about 90 μM, about 100 μM, about 200 μM, about 300 μM, about 400 μM, 500 μM, about 600 μM, about 700 μM, about 800 μM, about 900 μM, about 1000 μM, about 1500 μM or about 2000 μM.

As described above and herein, in some embodiments, R¹ represents independently for each occurrence halogen, —CN, —OR, C₁₋₆ alkyl, or C₁₋₆ haloalkyl. In some embodiments, R¹ represents independently for each occurrence halogen. In some embodiments, R¹ represents independently for each occurrence fluoro, chloro, or bromo. In some embodiments, R¹ represents independently for each occurrence —CN. In some embodiments, R¹ represents independently for each occurrence —OR. In some embodiments, R¹ represents independently for each occurrence C₁₋₆ alkyl or C₁₋₆ haloalkyl. In some embodiments, R¹ represents independently for each occurrence C₁₋₆ alkyl. In some such embodiments, R¹ represents independently for each occurrence methyl. In some embodiments, R¹ represents independently for each occurrence C₁₋₆ haloalkyl. In some such embodiments, R¹ represents independently for each occurrence —CF₃, —CF₂H, or —CFH₂. In some embodiments, at least one R¹ is fluoro. In some embodiments, at least one R¹ is chloro. In some embodiments, at least one R¹ is bromo. In some embodiments, at least one R¹ is —CN. In some embodiments, at least one R¹ is —OR. In some such embodiments, at least one R¹ is —OMe. In some embodiments, at least one R¹ is methyl. In some embodiments, at least one R¹ is —CF₃. In some embodiments, at least one R¹ is —CF₂H. In some embodiments, at least one R¹ is —CFH₂.

In some embodiments, R¹ is as depicted in Table 1, below.

In some embodiments, R¹ is as described above and herein, wherein m is 0 or 1. In some such embodiments, m is 0. In some such embodiments, m is 1.

As described above and herein, R² is C₁₋₆ aliphatic, phenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; hydrogen: or L¹-R⁴, wherein R² is substituted with p occurrences of R⁶.

In some embodiments, R² is C₁₋₆ aliphatic. In some such embodiments, R² is C₁₋₆ alkyl. In some such embodiments, R² is methyl, ethyl, or propyl. In some embodiments, R² is —CH₂CH(OH)CH₃.

In some embodiments, R² is phenyl. In some embodiments, R² is hydrogen.

In some embodiments, R² is a 3-7 membered saturated or partially unsaturated monocyclic carbocyclic ring. In some embodiments, R² is a 3-membered saturated monocyclic carbocyclic ring. In some embodiments, R² is a 4-membered saturated monocyclic carbocyclic ring. In some embodiments, R² is a 5-membered saturated monocyclic carbocyclic ring. In some embodiments, R² is a 6-membered saturated monocyclic carbocyclic ring. In some embodiments, R² is a 7-membered saturated monocyclic carbocyclic ring.

In some embodiments, R² is a 3-4 membered saturated monocyclic carbocyclic ring substituted with 0-2 occurrences of R⁶. In some embodiments, R² is a 3 membered saturated monocyclic carbocyclic ring substituted with 0-2 occurrences of R⁶. In some embodiments, R² is a 4 membered saturated monocyclic carbocyclic ring substituted with 0-2 occurrences of R⁶. In some embodiments, R² is a 3-4 membered saturated monocyclic carbocyclic ring substituted with 1-2 occurrences of R⁶. In some such embodiments, at least one R⁶ is fluoro. In some such embodiments, two R⁶ are fluoro. In some such embodiments, at least one R⁶ is methyl. In some such embodiments, at least one R⁶ is —CN.

In some embodiments, R² is a 5 membered saturated monocyclic carbocyclic ring substituted with 0-2 occurrences of R⁶. In some embodiments, R² is a 5 membered saturated monocyclic carbocyclic ring substituted with 1-2 occurrences of R⁶. In some such embodiments, at least one R⁶ is fluoro. In some such embodiments, two R⁶ are fluoro. In some such embodiments, at least one R⁶ is methyl. In some such embodiments, at least one R⁶ is —CN.

In some embodiments, R² is a 3-7 membered saturated or partially unsaturated monocyclic heterocyclic ring having 1-2 heteroatoms independently selected from nitrogen, oxygen, and sulfur. In some embodiments, R² is a 3 membered saturated monocyclic heterocyclic ring having 1-2 heteroatoms independently selected from nitrogen, oxygen, and sulfur. In some embodiments, R² is a 4 membered saturated monocyclic heterocyclic ring having 1-2 heteroatoms independently selected from nitrogen, oxygen, and sulfur. In some embodiments, R² is a 4 membered saturated monocyclic heterocyclic ring having 1 heteroatom independently selected from nitrogen, oxygen, and sulfur. In some embodiments, R² is a 4 membered saturated monocyclic heterocyclic ring having 1 heteroatom independently selected from nitrogen. In some embodiments, R² is a 5 membered saturated or partially unsaturated monocyclic heterocyclic ring having 1-2 heteroatoms independently selected from nitrogen, oxygen, and sulfur. In some embodiments, R² is a 6 membered saturated or partially unsaturated monocyclic heterocyclic ring having 1-2 heteroatoms independently selected from nitrogen, oxygen, and sulfur. In some embodiments, R² is a 7 membered saturated or partially unsaturated monocyclic heterocyclic ring having 1-2 heteroatoms independently selected from nitrogen, oxygen, and sulfur.

In some embodiments, R² is a 4-5 membered saturated monocyclic heterocyclic ring having 1-2 heteroatoms independently selected from nitrogen, oxygen, and sulfur substituted with 0-2 occurrences of R⁶. In some embodiments, R² is a 4-5 membered saturated monocyclic heterocyclic ring having 1-2 heteroatoms independently selected from nitrogen, oxygen, and sulfur substituted with 1-2 occurrences of R⁶. In some such embodiments, at least one R⁶ is fluoro. In some such embodiments, two R⁶ are fluoro. In some such embodiments, at least one R⁶ is methyl. In some such embodiments, at least one R⁶ is —CN.

In some embodiments, R² is a 5-6 membered monocyclic heteroaryl ring having 1-4 heteroatoms independently selected from nitrogen, oxygen, and sulfur. In some embodiments, R² is a 5 membered monocyclic heteroaryl ring having 1-4 heteroatoms independently selected from nitrogen, oxygen, and sulfur. In some embodiments, R² is a 6 membered monocyclic heteroaryl ring having 1-4 heteroatoms independently selected from nitrogen, oxygen, and sulfur.

In some embodiments, R² is an 8-10 membered bicyclic heteroaryl ring having 1-5 heteroatoms independently selected from nitrogen, oxygen, and sulfur. In some embodiments, R² is an 8-10 membered bicyclic heteroaryl ring having 1-3 heteroatoms independently selected from nitrogen, oxygen, and sulfur. In some embodiments, R² is an 8 membered bicyclic heteroaryl ring having 1-5 heteroatoms independently selected from nitrogen, oxygen, and sulfur. In some embodiments, R² is a 9 membered bicyclic heteroaryl ring having 1-5 heteroatoms independently selected from nitrogen, oxygen, and sulfur. In some embodiments, R² is a 10 membered bicyclic heteroaryl ring having 1-5 heteroatoms independently selected from nitrogen, oxygen, and sulfur.

In some embodiments, R² is a 5-10 membered saturated or partially unsaturated bridged bicyclic ring having 0-3 heteroatoms independently selected from nitrogen, oxygen, and sulfur. In some embodiments, R² is a 5-10 membered saturated or partially unsaturated bridged bicyclic ring having 1-3 heteroatoms independently selected from nitrogen, oxygen, and sulfur. In some embodiments, R² is a 5-10 membered saturated bridged bicyclic ring having 0-3 heteroatoms independently selected from nitrogen, oxygen, and sulfur. In some embodiments, R² is a 5-10 membered partially unsaturated bridged bicyclic ring having 0-3 heteroatoms independently selected from nitrogen, oxygen, and sulfur. In some embodiments, R² is a 5-10 membered saturated or partially unsaturated bridged bicyclic ring having 0 heteroatoms. In some embodiments, R² is a 5-10 membered saturated or partially unsaturated bridged bicyclic ring having one heteroatom independently selected from nitrogen, oxygen, and sulfur. In some embodiments, R² is a 5-10 membered saturated or partially unsaturated bridged bicyclic ring having two heteroatoms independently selected from nitrogen, oxygen, and sulfur. In some embodiments, R² is a 5-10 membered saturated or partially unsaturated bridged bicyclic ring having three heteroatoms independently selected from nitrogen, oxygen, and sulfur.

In some embodiments, R² is a 6-11 membered saturated or partially unsaturated spirocyclic ring having 0-3 heteroatoms independently selected from nitrogen, oxygen, and sulfur. In some embodiments, R² is a 6-11 membered saturated or partially unsaturated spirocyclic ring having 0 heteroatoms. In some embodiments, R² is a 6-11 membered saturated or partially unsaturated spirocyclic ring having 1-3 heteroatoms independently selected from nitrogen, oxygen, and sulfur.

In some embodiments, R² is a 6-11 membered saturated or partially unsaturated bicyclic heterocyclic ring having 1-3 heteroatoms independently selected from nitrogen, oxygen, and sulfur.

In some embodiments, R² is L¹-R⁴.

As described above and defined herein, L¹ represents is a C₁₋₃ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₂ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—.

As described above and defined herein, R⁴ is a 3-7 membered saturated or partially unsaturated monocyclic carbocyclic ring, a 3-7 membered saturated or partially unsaturated monocyclic heterocyclic ring having 1-2 heteroatoms independently selected from nitrogen, oxygen, and sulfur, or optionally substituted phenyl. In some embodiments, R⁴ is a 3-7 membered saturated or partially unsaturated monocyclic carbocyclic ring. In some embodiments, R⁴ is a 3-7 membered saturated or partially unsaturated monocyclic heterocyclic ring having 1-2 heteroatoms independently selected from nitrogen, oxygen, and sulfur. In some embodiments, R⁴ is optionally substituted phenyl.

As described above and herein, R⁶ represents independently for each occurrence oxo, halogen, C₁₋₆ aliphatic, C₁₋₆ haloaliphatic, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or optionally substituted phenyl.

In some embodiments, at least one R⁶ is halogen. In some embodiments, at least one R⁶ is fluoro. In some embodiments, at least two R⁶ are fluoro. In some embodiments, at least one R⁶ is methyl. In some embodiments, at least one R⁶ is cyano.

In some embodiments, R² is selected from:

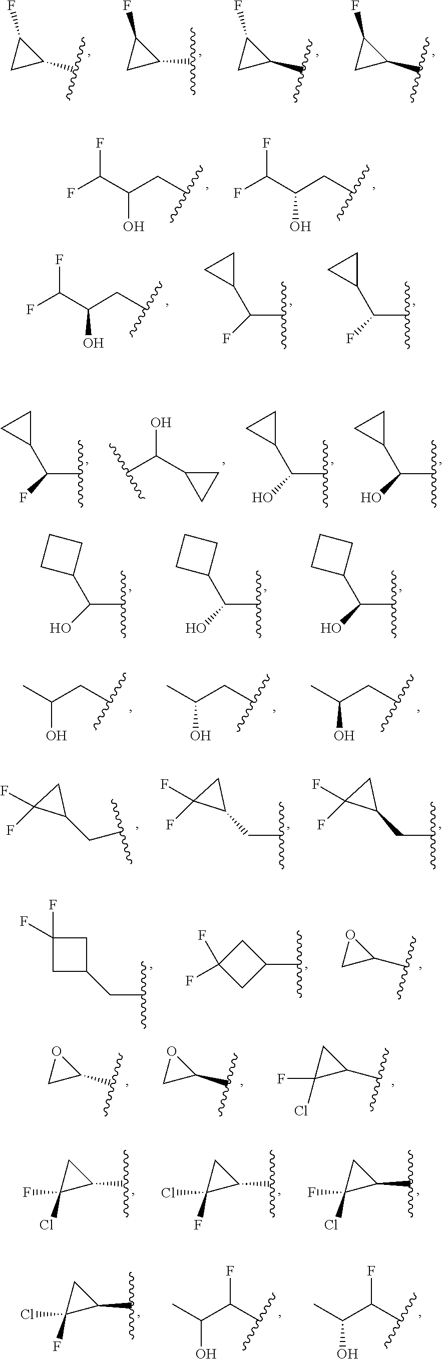

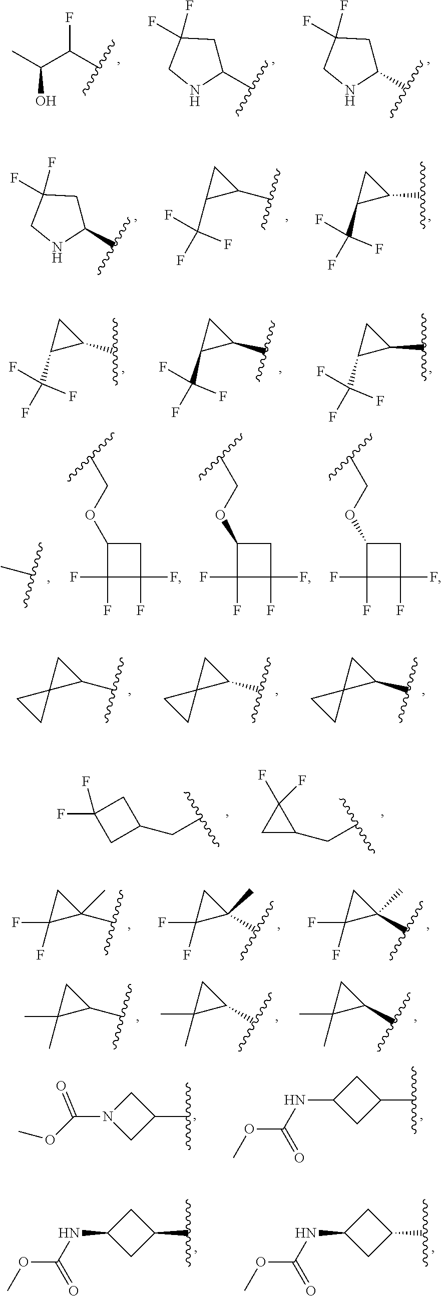

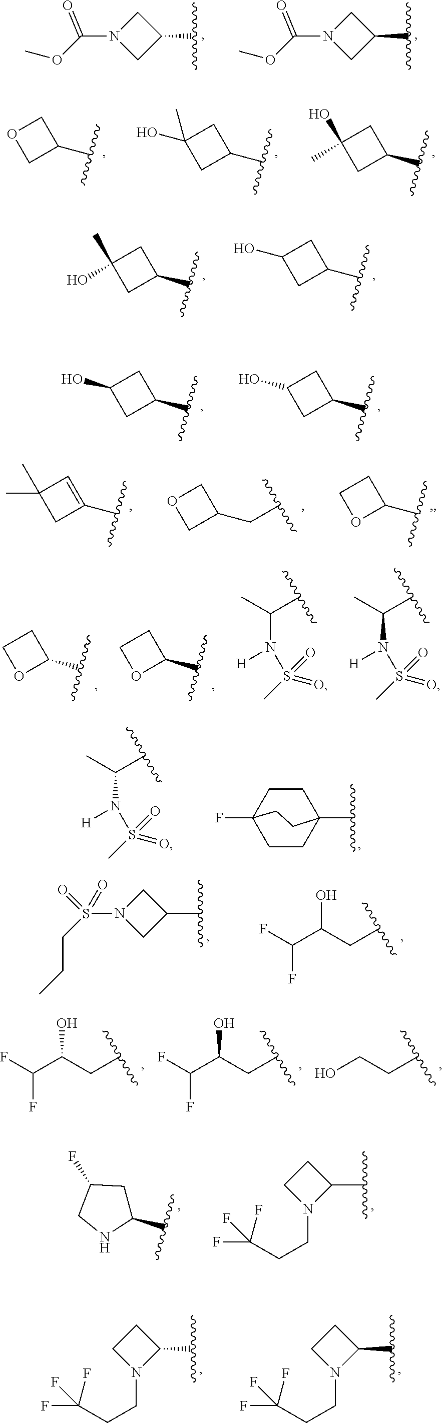

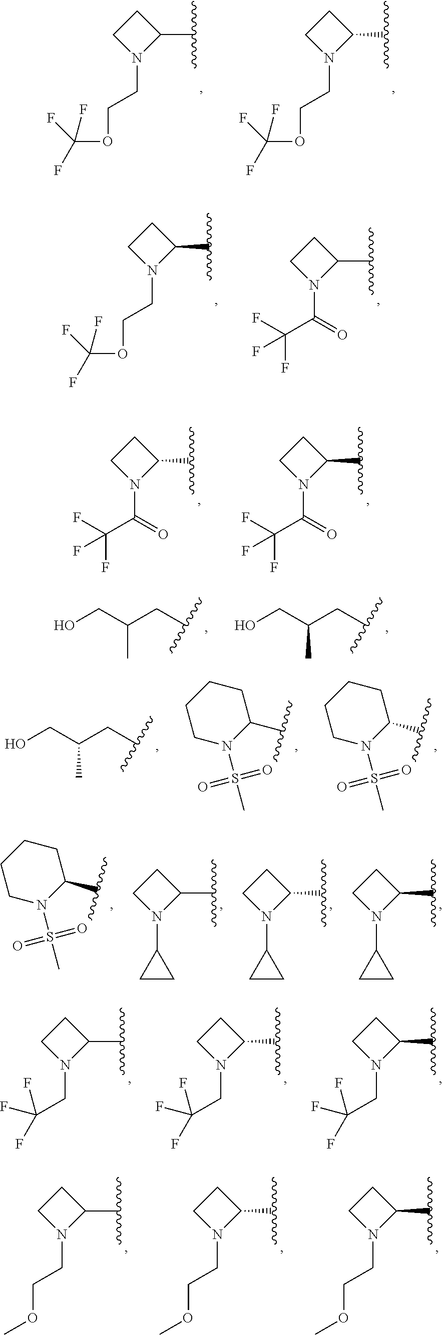

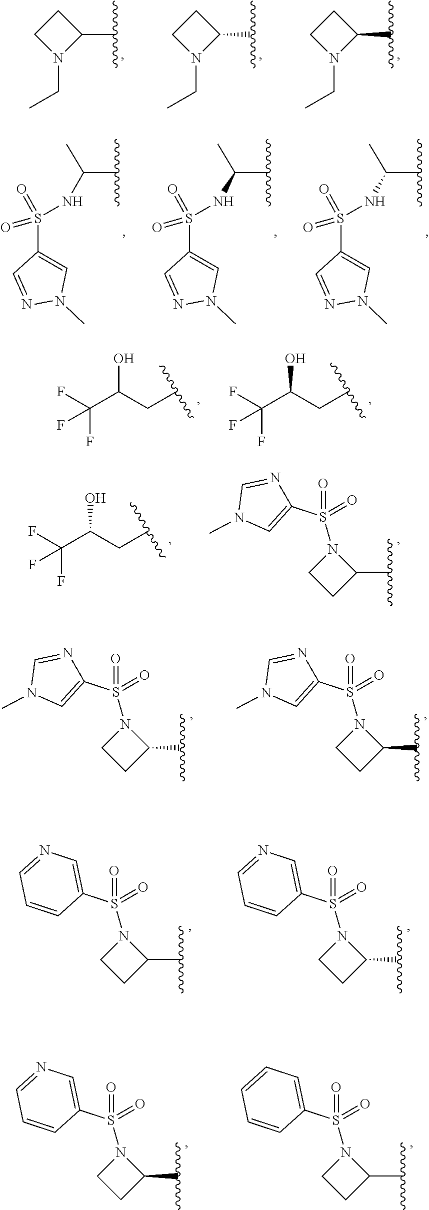

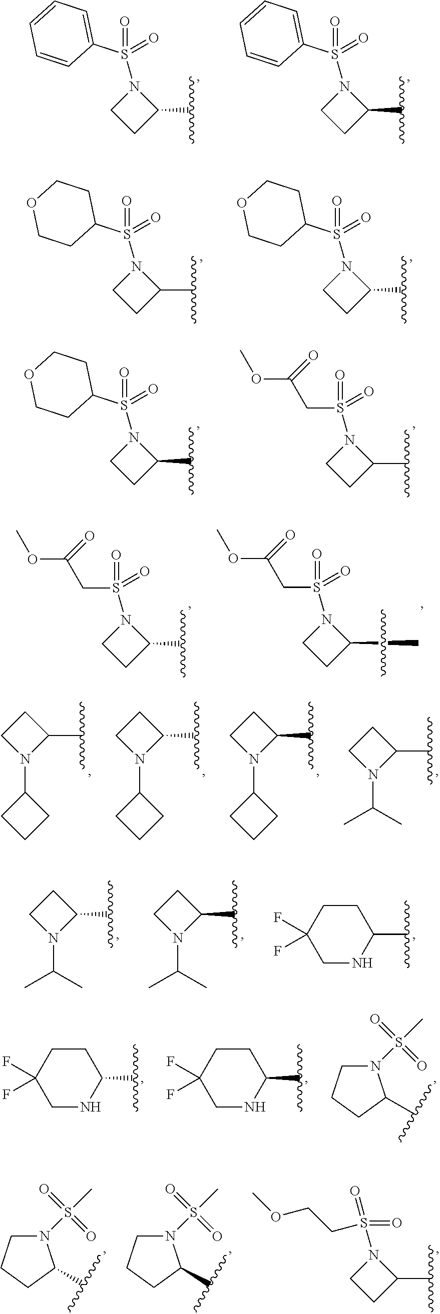

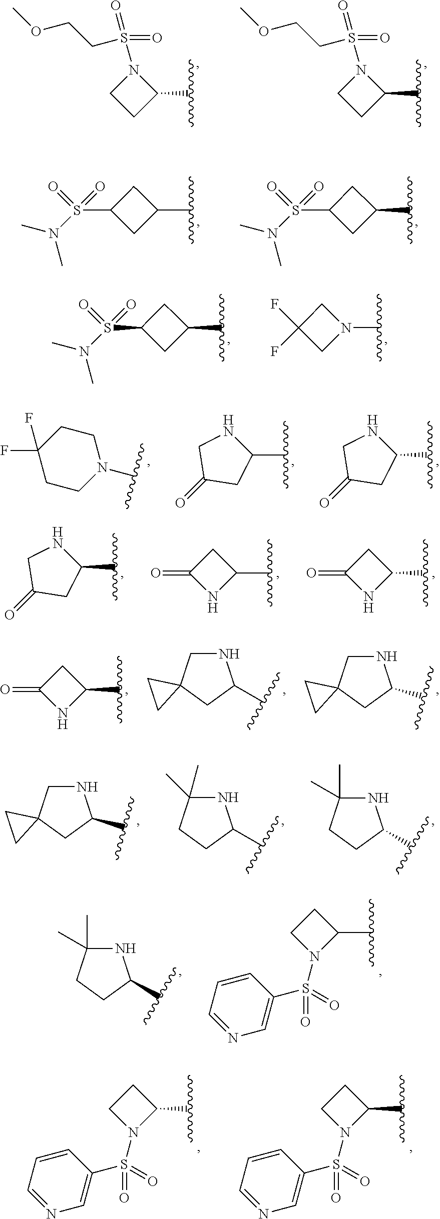

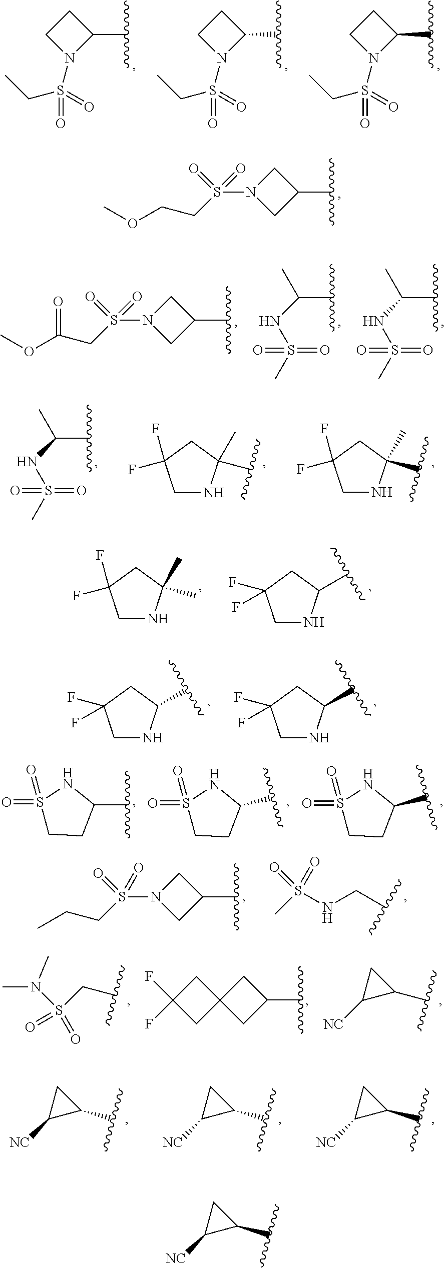

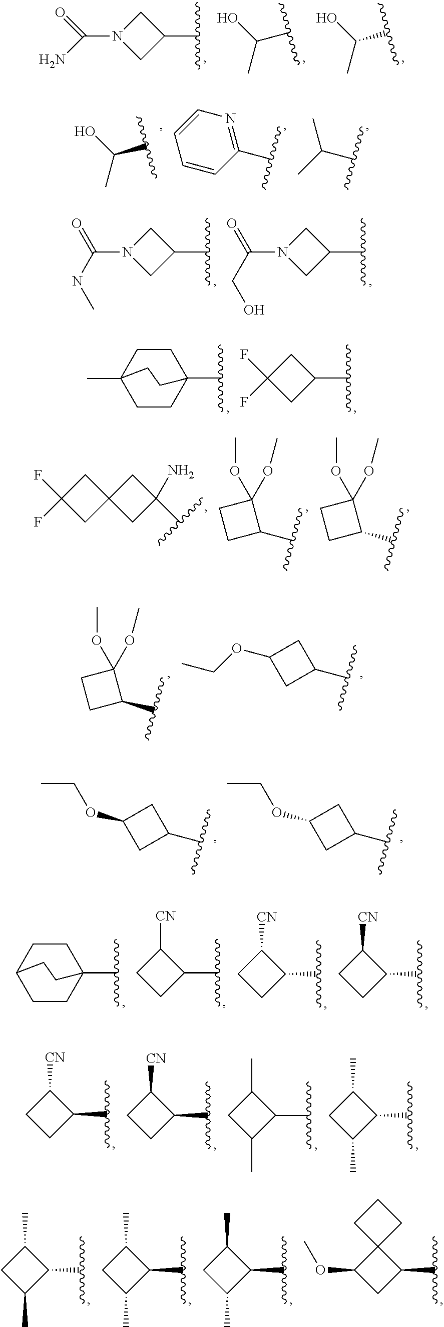

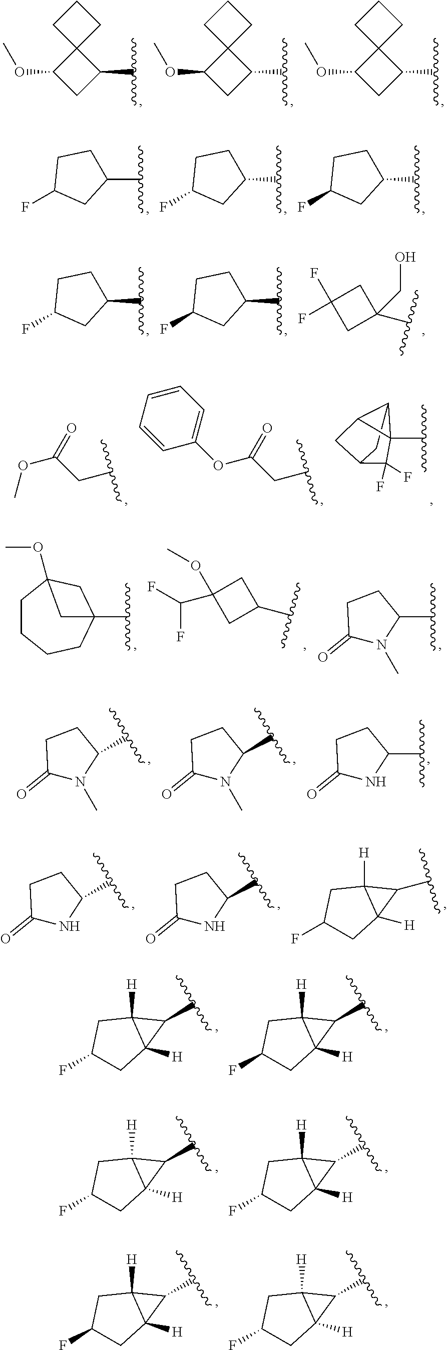

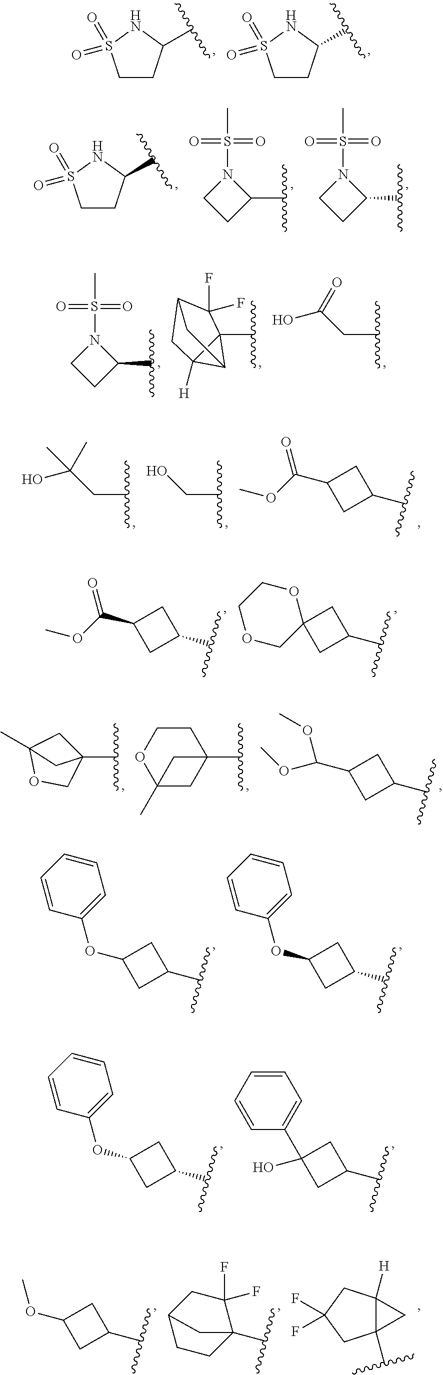

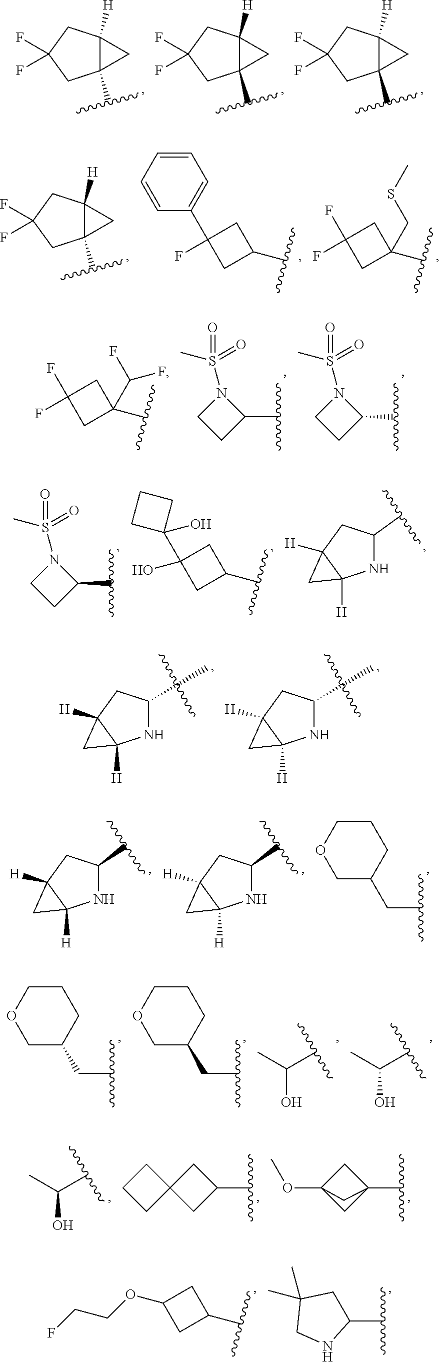

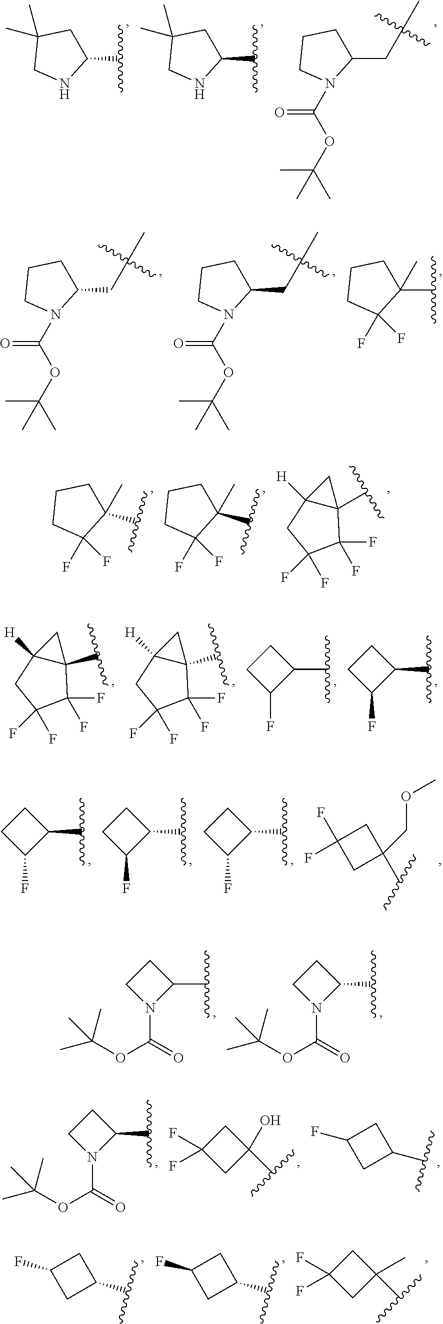

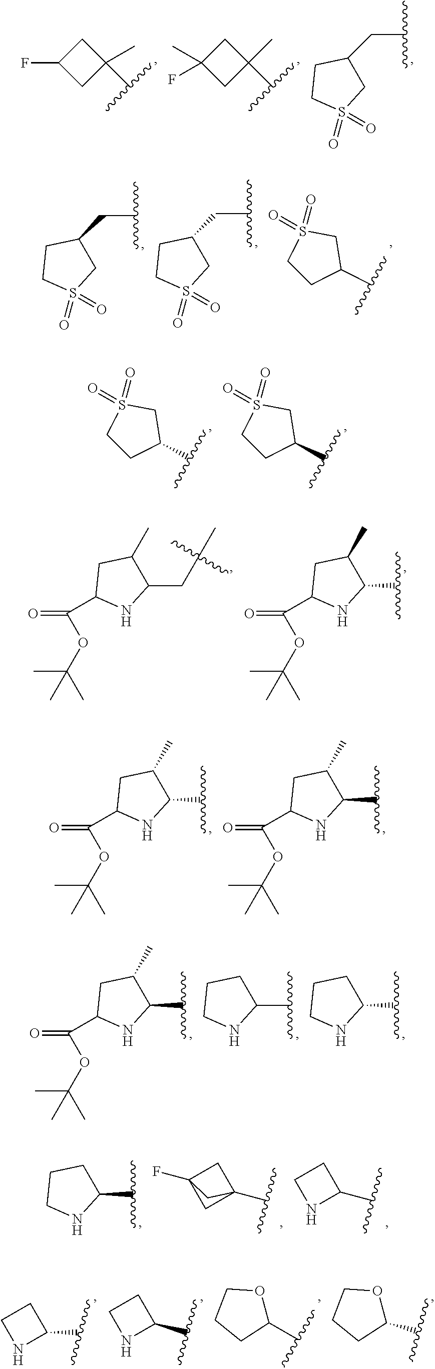

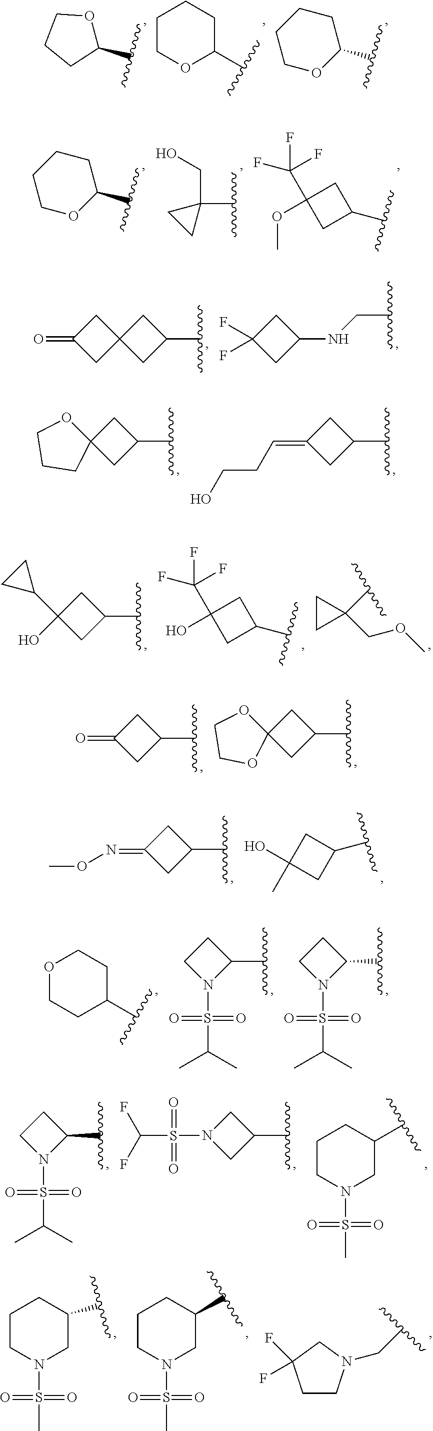

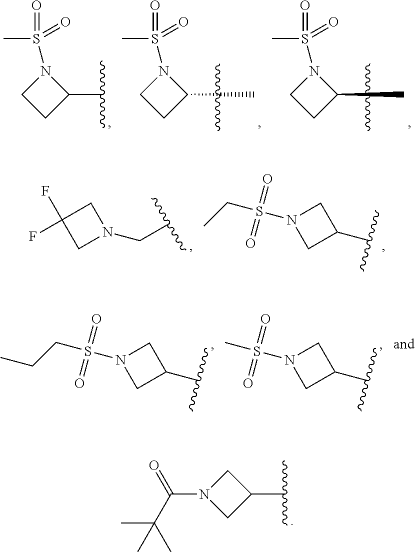

In some embodiments, R² is selected from:

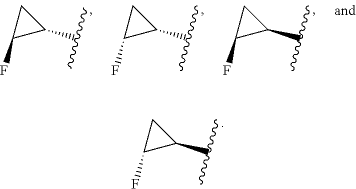 [SMILES: *[C@H]1C[C@@H]1F ; *[C@H]1C[C@H]1F ; *[C@@H]1C[C@@H]1F ; *[C@@H]1C[C@H]1F]

In some embodiments, R^{Z} is selected from:

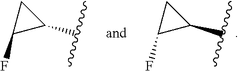 [SMILES: *[C@H]1C[C@@H]1F ; *[C@@H]1C[C@H]1F]

In some embodiments, R² is:

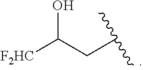 [SMILES: *CC(O)C(F)F]

In some embodiments, R² is selected from:

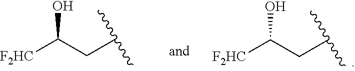 [SMILES: *C[C@H](O)C(F)F ; *C[C@@H](O)C(F)F]

In some embodiments, R² is as described above and herein, wherein R² is substituted with p occurrences of R⁶. In some such embodiments, p is 0. In some such embodiments, p is 1. In some such embodiments, p is 2. In some such embodiments, p is 3. In some such embodiments, p is 4. In some such embodiments, p is 5.

In some embodiments, R² is as depicted in Table 1, below.

As described above and herein, R^{A} is of any of the following structures:

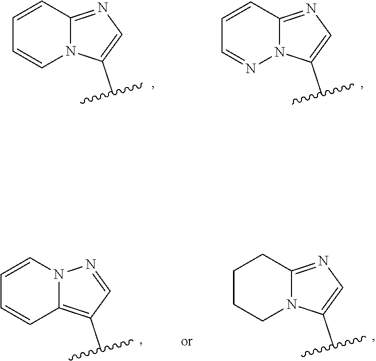 [SMILES: *c1cnc2ccccn12 ; *c1cnc2cccnn12 ; *c1cnn2ccccc12 ; *c1cnc2n1CCCC2]

each of which is substituted by n occurrences of R³.

In some embodiments, R^{A} is

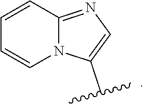 [SMILES: *c1cnc2ccccn12]

In some embodiments, R^{A} is

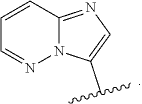 [SMILES: *c1cnc2cccnn12]

In some embodiments, R^{A} is

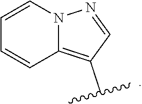 [SMILES: *c1cnn2ccccc12]

In some embodiments, R^{A} is

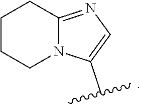 [SMILES: *c1cnc2n1CCCC2]

In some embodiments, R^{A} is any of those depicted in Table 1 below.

As described above and herein, each R^{A} is substituted by n occurrences of R³. In some embodiments, n is 0. In some embodiments, n is 1. In some embodiments, n is 2. In some embodiments, n is 3. In some embodiments, n is 4. In some embodiments, n is 5.

As described above and herein, R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(R)₂OCR₃, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, -L²-R⁵, or an optionally substituted group selected from C₁₋₆ aliphatic, C₁₋₆ haloaliphatic, phenyl, naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; each of which is substituted with r instances of R; or:
two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur, each of which is substituted with r instance of R.

In some embodiments, R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or -L²-R⁵. In some embodiments, R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(R)₂OCR₃, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or -L²-R⁵.

In some embodiments, R³ represents independently for each occurrence halogen. In some embodiments, at least one R³ is fluoro. In some embodiments, at least one R³ is chloro. In some embodiments, at least one R³ is bromo. In some embodiments, at least one R³ is cyano. In some embodiments, at least one R³ is —OR. In some embodiments, wherein at least one R³ is —OR, wherein R is C₁₋₆ alkyl. In some embodiments, at least one R³ is —OR, wherein R is methyl, ethyl, or propyl. In some embodiments, at least one R³ is —OR, wherein R is methyl. In some embodiments, at least one R³ is —OR, wherein R is ethyl. In some embodiments, at least one R³ is —OR, wherein R is propyl. In some embodiments, at least one R³ is —OCR₃, wherein at least one R is fluoro. In some embodiments, at least one R³ is —NR₂. In some embodiments, wherein at least one R³ is —NR₂, at least one R is hydrogen. In some embodiments, wherein at least one R³ is —NR₂, at least one R is methyl or ethyl. In some embodiments, wherein at least one R³ is —NR₂, at least one R is optionally substituted phenyl. In some embodiments, wherein at least one R³ is —NR₂, the two R groups on the same nitrogen are taken together with the nitrogen to form an optionally substituted 4-7 membered monocyclic saturated ring having 1-3 heteroatoms independently selected from nitrogen, oxygen, and sulfur. In some embodiments, at least one R³ is —N(R)S(O)₂R. In some such embodiments, each R is independently hydrogen, C₁₋₆ alkyl, C₃₋₆ cycloalkyl, naphthalenyl, or a 5-membered heteroaryl ring having one, two, or three heteroatoms independently selected from nitrogen, oxygen, or sulfur.

In some embodiments, at least one R³ is -L²-R⁵. In some such embodiments, one, two, or three methylene units of L² are independently replaced by —O— or -Cy-. In some such embodiments, one, two, or three methylene units of L² are independently replaced by —N(R)— or -Cy-.

In some embodiments, at least one R³ is a 3-7 membered saturated or partially unsaturated monocyclic heterocyclic ring having 1-2 heteroatoms independently selected from nitrogen, oxygen, and sulfur. In some such embodiments, at least one R³ is oxetane.

In some embodiments, at least one R³ is —CF₃, —CF₂H, or —CFH₂.

In some embodiments, R³ represents independently for each occurrence C₁₋₆ aliphatic or C₁₋₆ haloaliphatic.

In some embodiments, R³ represents independently for each occurrence phenyl or naphthalenyl.

In some embodiments, R³ represents independently for each occurrence a 3-7 membered saturated or partially unsaturated monocyclic carbocyclic ring.

In some embodiments, R³ represents independently for each occurrence a 3-7 membered saturated or partially unsaturated monocyclic heterocyclic ring having 1-2 heteroatoms independently selected from nitrogen, oxygen, and sulfur.

In some embodiments, R³ represents independently for each occurrence a 5-6 membered monocyclic heteroaryl ring having 1-4 heteroatoms independently selected from nitrogen, oxygen, and sulfur.

In some embodiments, R³ represents independently for each occurrence an 8-10 membered bicyclic heteroaryl ring having 1-5 heteroatoms independently selected from nitrogen, oxygen, and sulfur.

In some embodiments, R³ represents independently for each occurrence a 5-8 membered saturated or partially unsaturated bridged bicyclic ring having 0-3 heteroatoms independently selected from nitrogen, oxygen, and sulfur.

In some embodiments, R³ represents independently for each occurrence a 6-11 membered saturated or partially unsaturated spirocyclic ring having 0-3 heteroatoms independently selected from nitrogen, oxygen, and sulfur.

In some embodiments, R³ represents independently for each occurrence or a 6-11 membered saturated or partially unsaturated bicyclic heterocyclic ring having 1-3 heteroatoms independently selected from nitrogen, oxygen, and sulfur.

In some embodiments, two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur. In some embodiments, two R³ groups on adjacent carbon atoms are taken together with the carbon atoms to which they attach to form an optionally substituted 4-7 membered saturated monocyclic ring having 1-3 heteroatoms independently selected from nitrogen, oxygen, and sulfur. In some embodiments, two R³ groups on adjacent carbon atoms are taken together with the carbon atoms to which they attach to form an optionally substituted 5 membered saturated monocyclic ring having 1-3 heteroatoms independently selected from nitrogen, oxygen, and sulfur. In some embodiments, two R³ groups on adjacent carbon atoms are taken together with the carbon atoms to which they attach to form an optionally substituted 4-7 membered saturated monocyclic ring having one heteroatom independently selected from oxygen.

In some embodiments, R³ represents independently for each occurrence -L²-R⁵.

As described above and herein, each L² represents independently for each occurrence a C₁₋₆ bivalent saturated or unsaturated, straight or branched hydrocarbon chain wherein one, two, or three methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂— or -Cy-. In some embodiments, each L² represents independently for each occurrence a C₁₋₆ bivalent saturated or straight or branched hydrocarbon chain wherein one, two, or three methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂— or -Cy-.

As described above and herein, -Cy- represents independently for each occurrence phenyl, a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur.

In some embodiments, -Cy- represents independently for each occurrence phenyl.

In some embodiments, -Cy- represents independently for each occurrence a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, -Cy- represents independently for each occurrence a 3-7 membered saturated monocyclic carbocyclic ring. In some embodiments, -Cy- represents independently for each occurrence a 3-7 membered saturated or partially unsaturated monocyclic heterocyclic ring having 1-2 heteroatoms independently selected from nitrogen, oxygen, and sulfur.

As described above and herein, R⁵ represents independently for each occurrence hydrogen, OR, C₁₋₆ aliphatic, C₁₋₆ haloaliphatic, or phenyl fused to a 5-6 membered saturated heterocyclic ring having 1-2 heteroatoms independently selected from nitrogen, oxygen, and sulfur. In some embodiments, R⁵ represents independently for each occurrence hydrogen. In some embodiments, R⁵ represents independently for each occurrence OR. In some such embodiments, R⁵ represents independently for each occurrence OH or OMe. In some embodiments, R⁵ represents independently for each occurrence C₁₋₆ aliphatic or C₁₋₆ haloaliphatic. In some embodiments, R⁵ represents independently for each occurrence phenyl fused to a 5-6 membered saturated heterocyclic ring having 1-2 heteroatoms independently selected from nitrogen, oxygen, and sulfur.

In some embodiments, each R³ group is independently substituted with r instances of R. In some embodiments, r is 0. In some embodiments, r is 1. In some embodiments, r is 2. In some embodiments, r is 3. In some embodiments, r is 4. In some embodiments, r is 5.

In some embodiments, R³ is independently for each occurrence selected from:

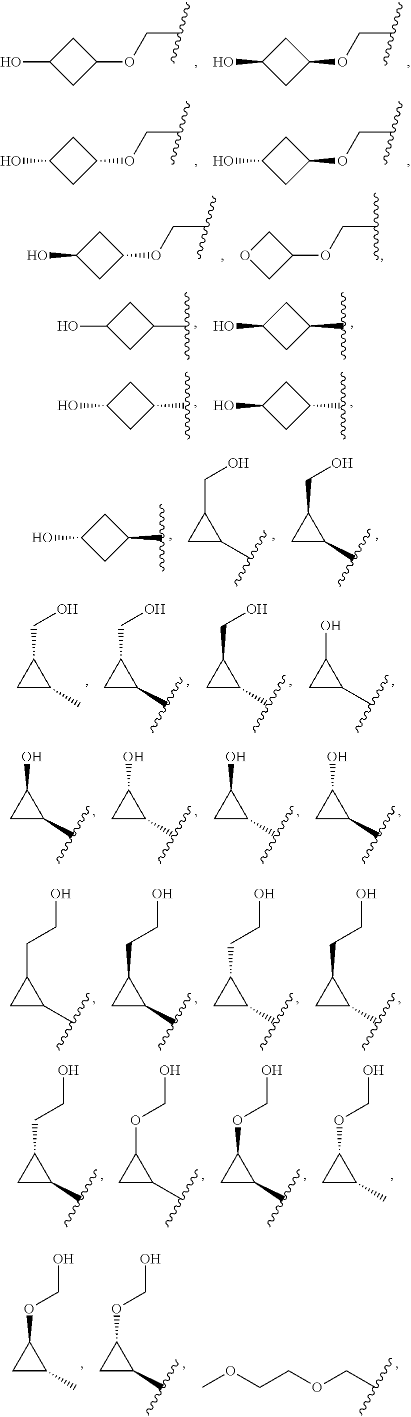

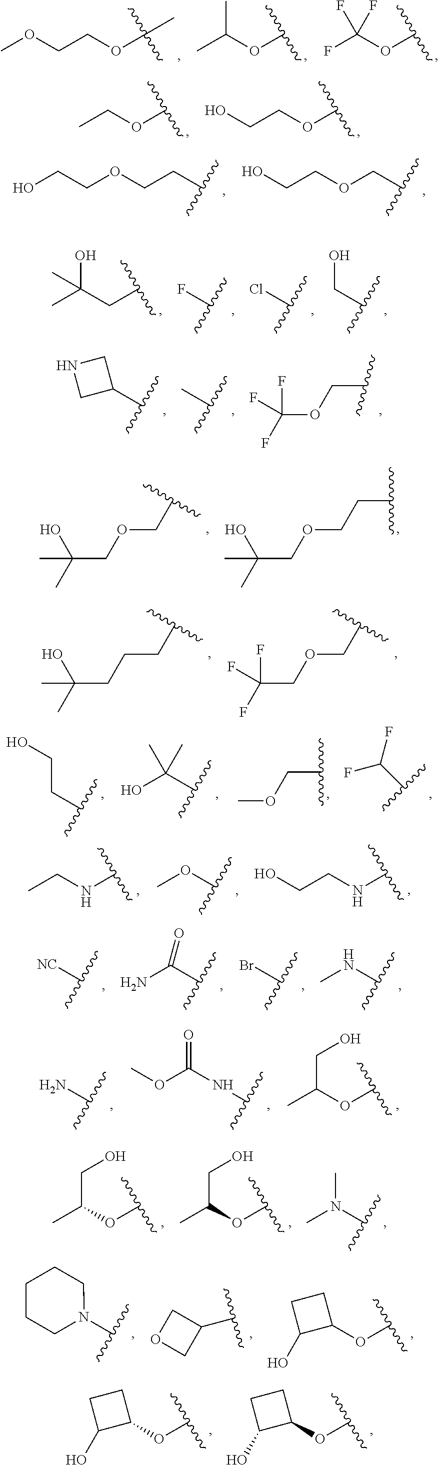

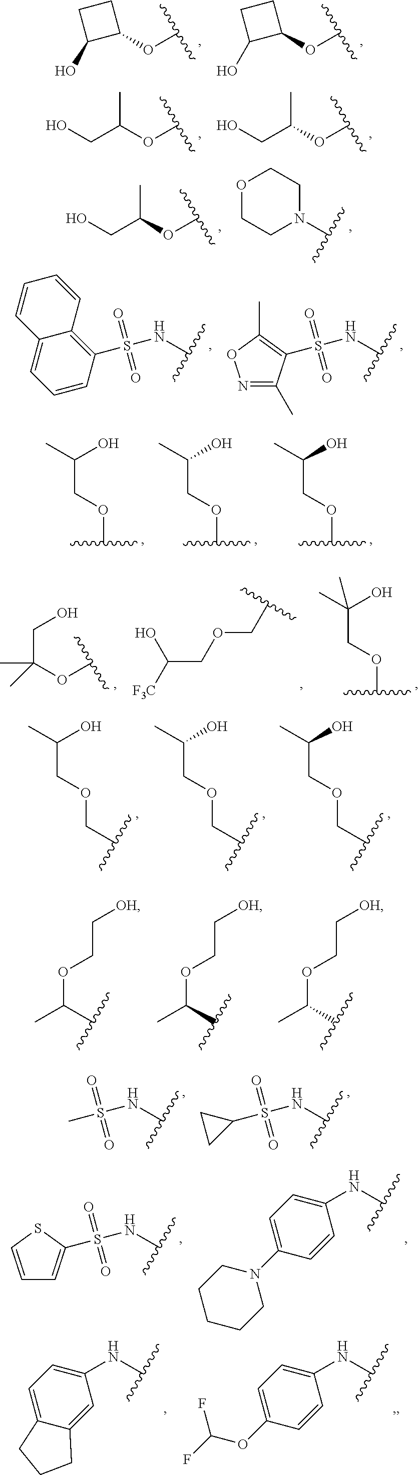

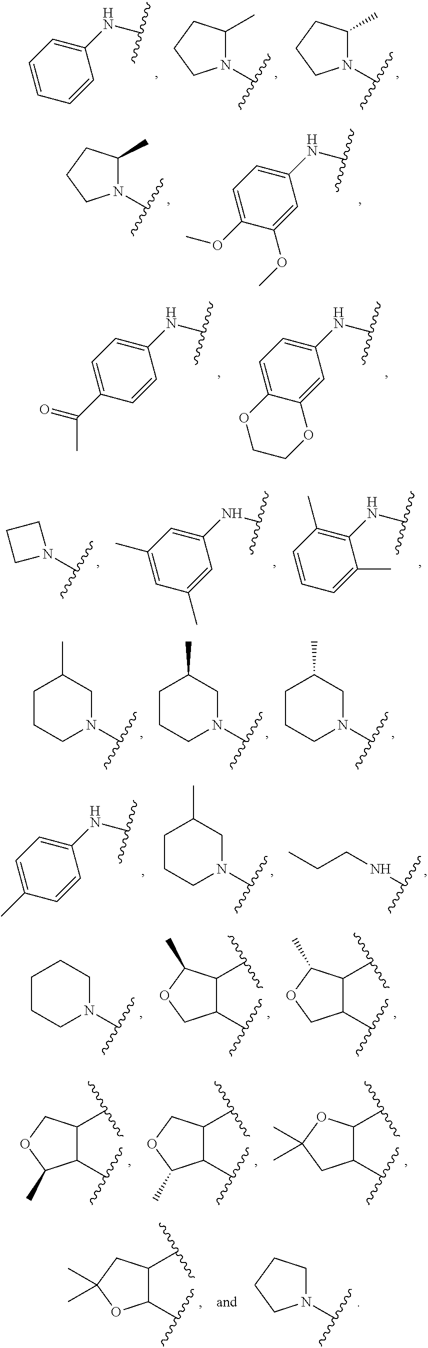

In some embodiments, each R³ is independently selected from:

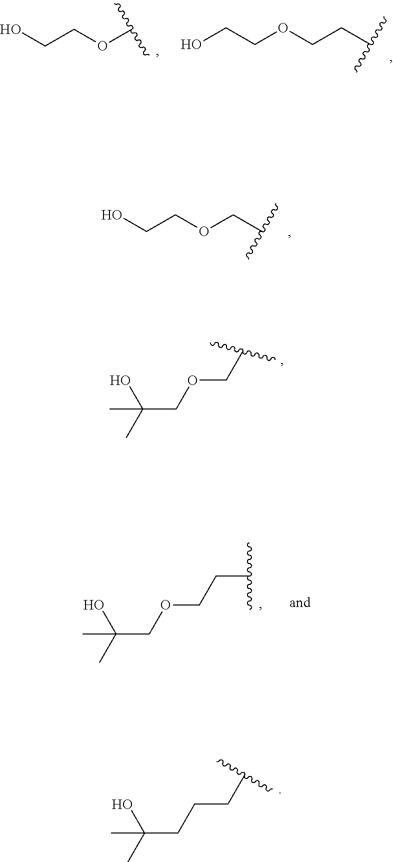

In some embodiments, each R³ is independently selected from:

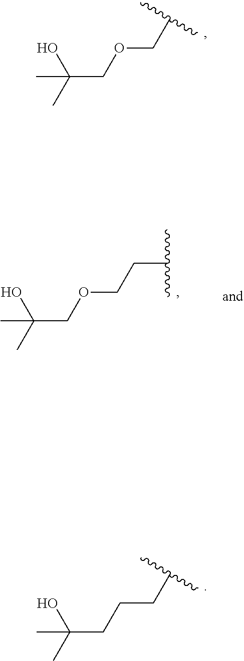

In some embodiments, each R³ is independently selected from:

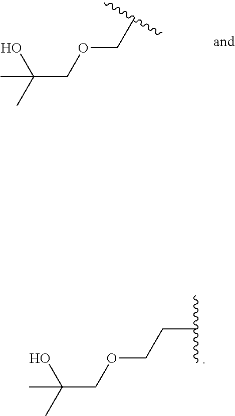

In some embodiments, R³ is —OMe or -OiPr.

In some embodiments, R³ is as depicted in Table 1 below.

As defined above and herein, R is independently for each occurrence hydrogen, —CN, halogen, oxo, or an optionally substituted group selected from C₁₋₆ aliphatic; C₁₋₆ haloaliphatic; C₁₋₃ hydroxyalkyl; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are independently taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur.

In some embodiments, R is independently for each occurrence hydrogen, —CN, halogen, or an optionally substituted group selected from C₁₋₆ aliphatic or C₁₋₆ haloaliphatic. In some embodiments, R is independently for each occurrence hydrogen. In some embodiments, R is independently for each occurrence halogen, for instance fluoro. In some embodiments, R is independently for each occurrence an optionally substituted group selected from C₁₋₆ alkyl. In some such embodiments, R is independently for each occurrence methyl.

In some embodiments, R is independently for each occurrence an optionally substituted group selected from phenyl or naphthalenyl.

In some embodiments, R is independently for each occurrence an optionally substituted group selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic carbocyclic ring having 1-2 heteroatoms independently selected from nitrogen, oxygen, and sulfur.

In some embodiments, two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur.

In some embodiments, a compound of the present disclosure is represented by any of the following or a pharmaceutically acceptable salt thereof:

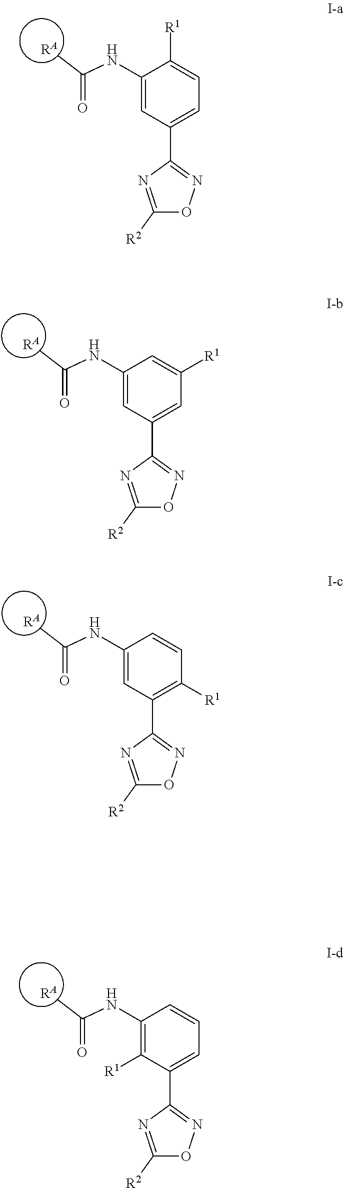

wherein R^{A}, R¹, and R² are as defined above and described herein.

In some embodiments, a compound of the present disclosure is represented by either of the following or a pharmaceutically acceptable salt thereof:

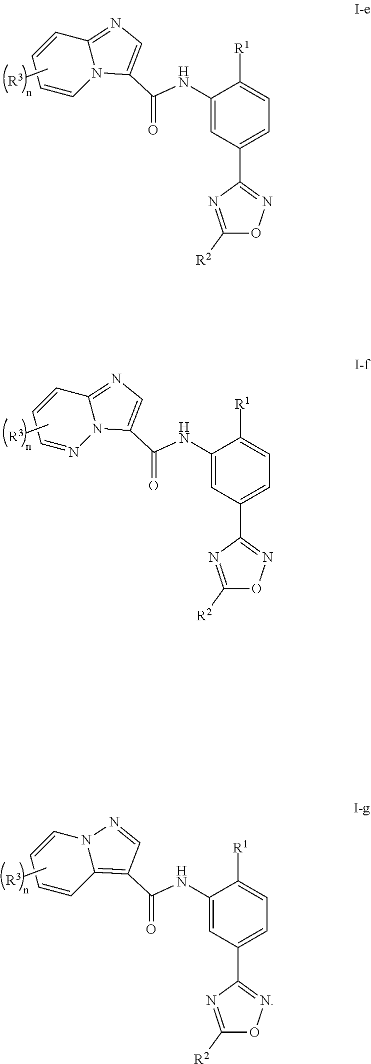

wherein R¹, R², R³, and n are as defined above and described herein.

In some embodiments, a compound of the present disclosure is represented by either of the following or a pharmaceutically acceptable salt thereof:

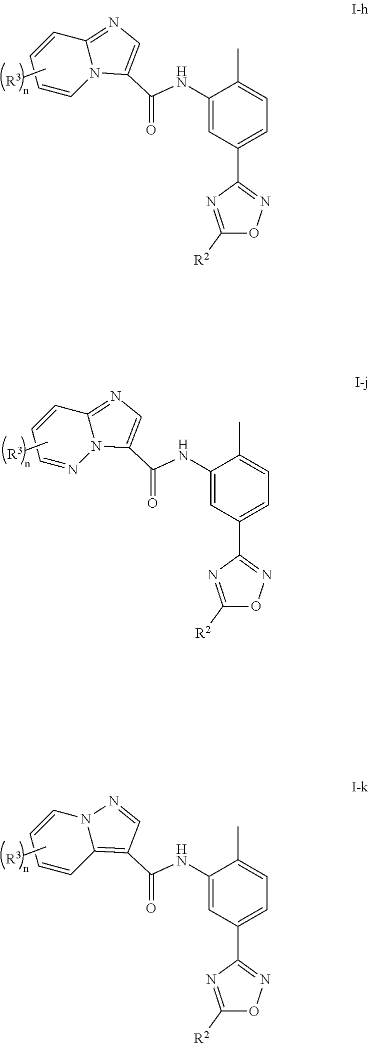

wherein R², R³, and n are as defined above and described herein.

In some embodiments, a compound of the present disclosure is represented by either of the following or a pharmaceutically acceptable salt thereof:

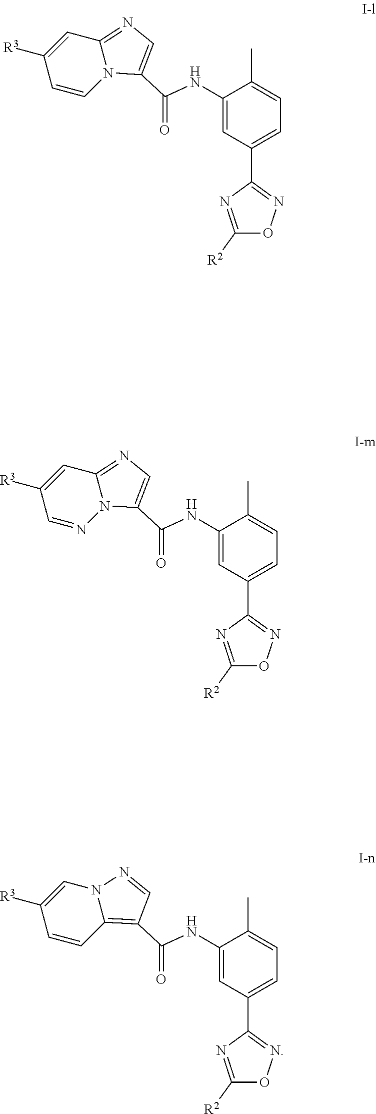

wherein R² and R³ are as defined above and described herein. In some such embodiments, R³ is -OiPr, —CH₂O(CH₂)₂OH, or —CH₂OCH₂C(CH₃)₂OH, and R² is selected from

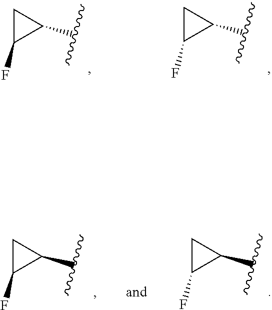 [SMILES: *[C@H]1C[C@@H]1F ; *[C@H]1C[C@H]1F ; *[C@@H]1C[C@@H]1F ; *[C@@H]1C[C@H]1F]

In some embodiments, a compound of the present disclosure is represented by either of the following or a pharmaceutically acceptable salt thereof:

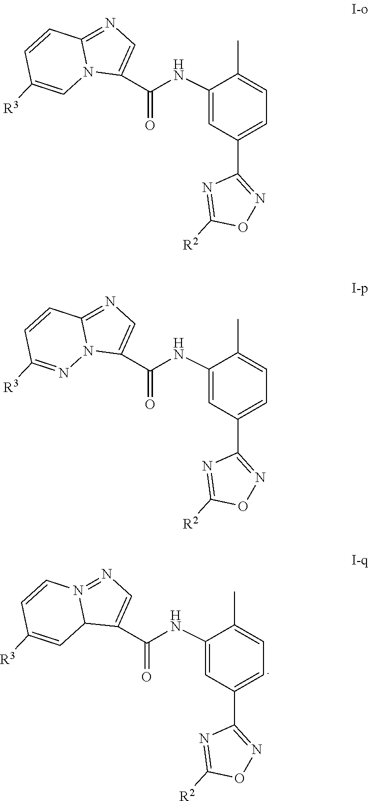

wherein R² and R³ are as defined above and described herein.

In some embodiments, a compound of the present disclosure is represented by either of the following or a pharmaceutically acceptable salt thereof:

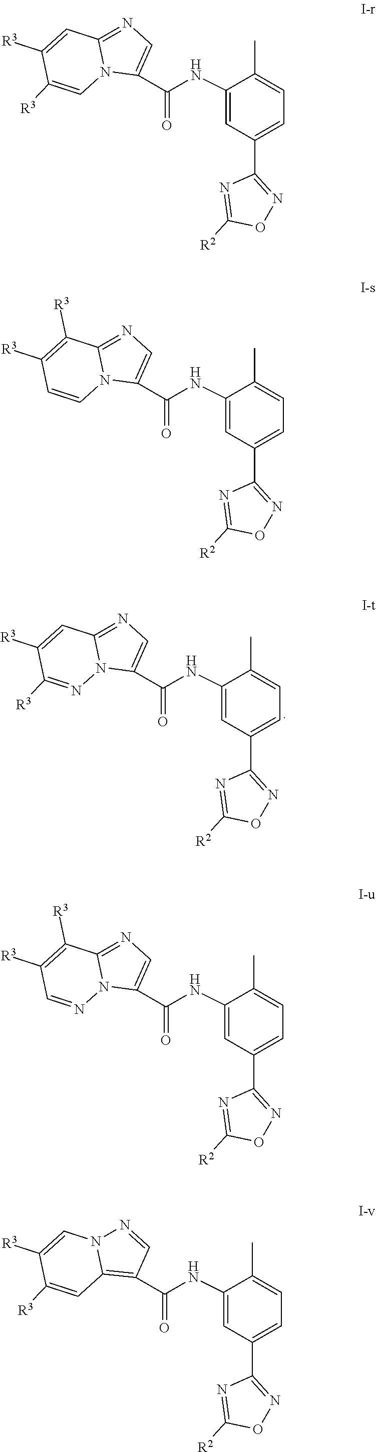

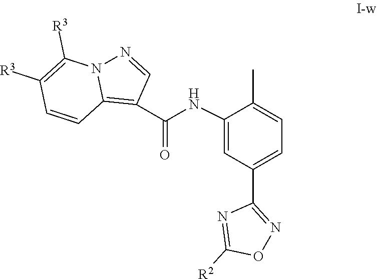

wherein R² and R³ are as defined above and described herein.

In some embodiments, a compound of the present disclosure is represented by either of the following or a pharmaceutically acceptable salt thereof:

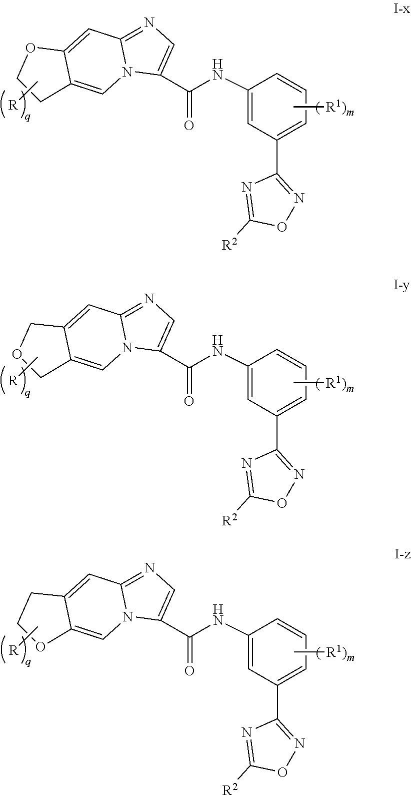

wherein R¹, R², R, and m are as defined above and described herein and q is 0, 1, 2, 3 or 4.

In some embodiments, a compound of the present disclosure is represented by either of the following or a pharmaceutically acceptable salt thereof:

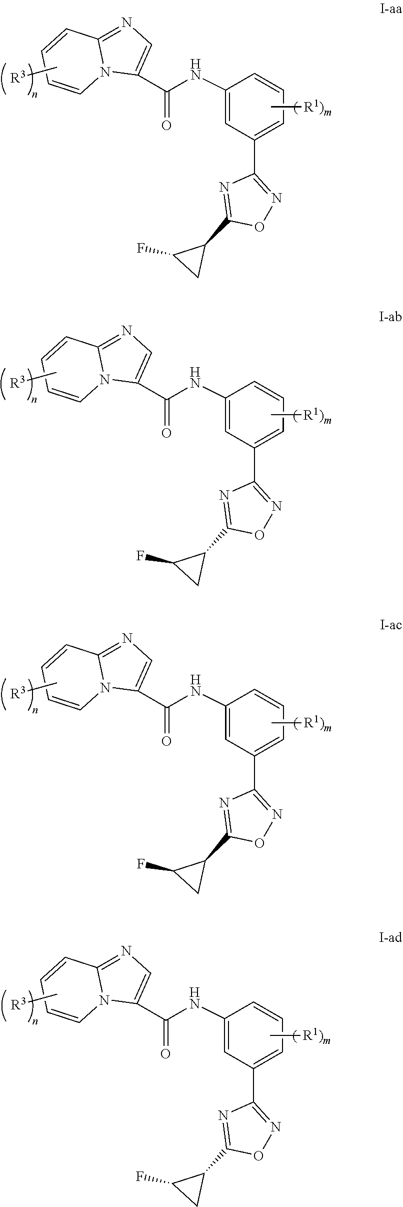

wherein R¹, R³, m, and n are as defined above and described herein.

In some embodiments, a compound of the present disclosure is represented by either of the following or a pharmaceutically acceptable salt thereof:

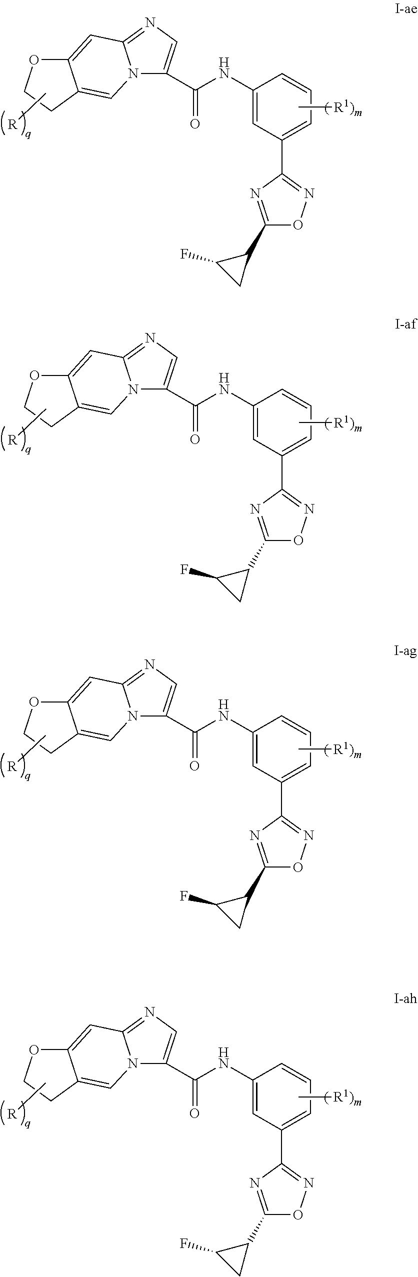

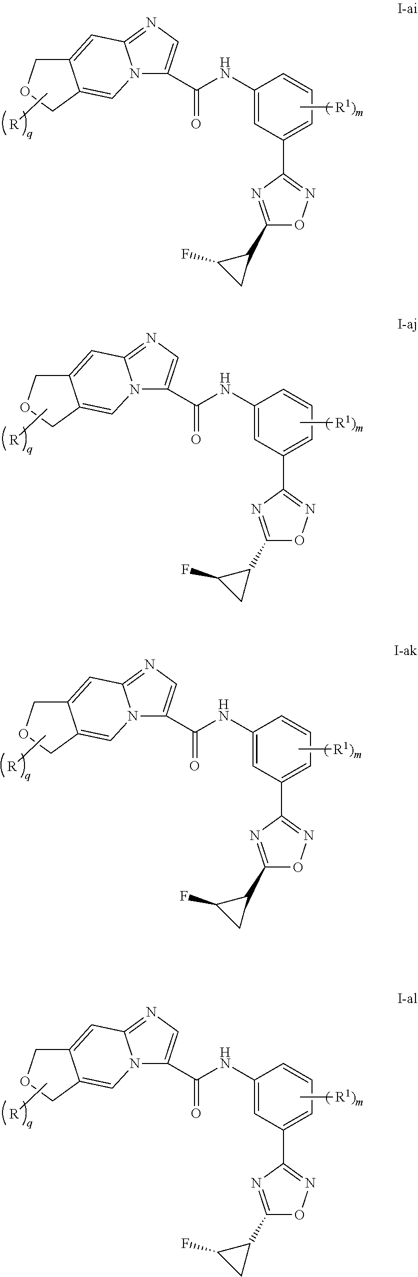

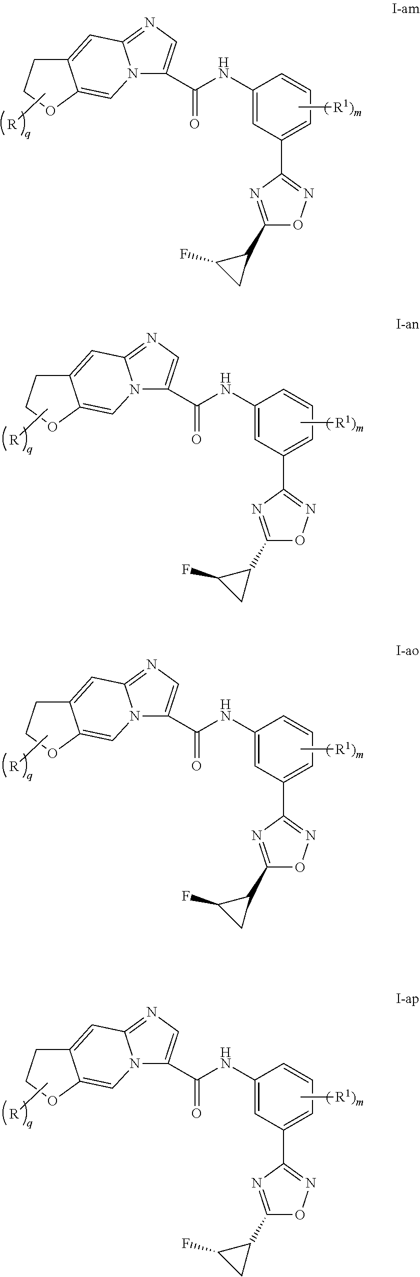

wherein R¹, R, m, and q are as defined above and described herein.

In some embodiments, a compound of the present disclosure is represented by any of the following or a pharmaceutically acceptable salt thereof:

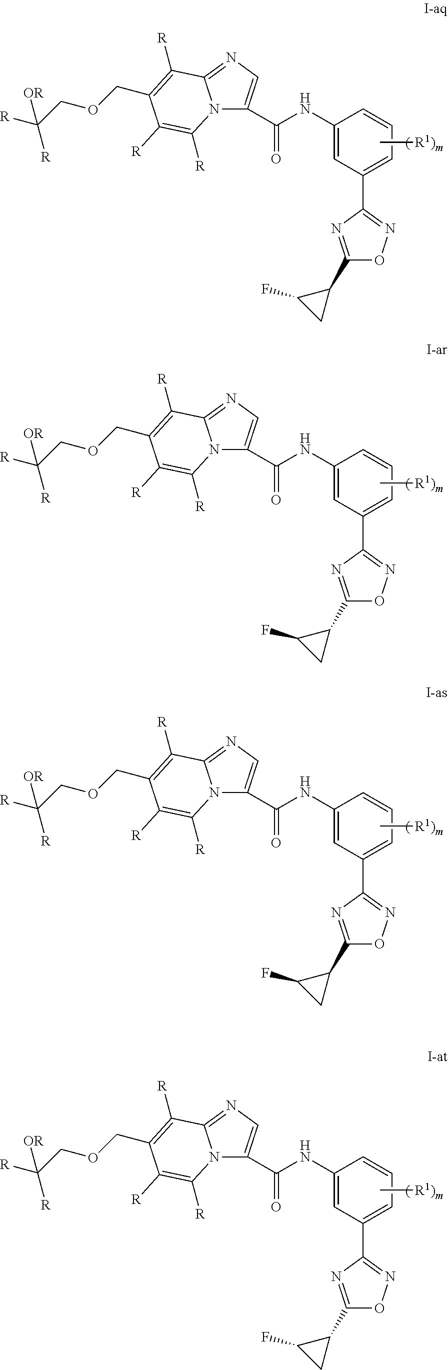

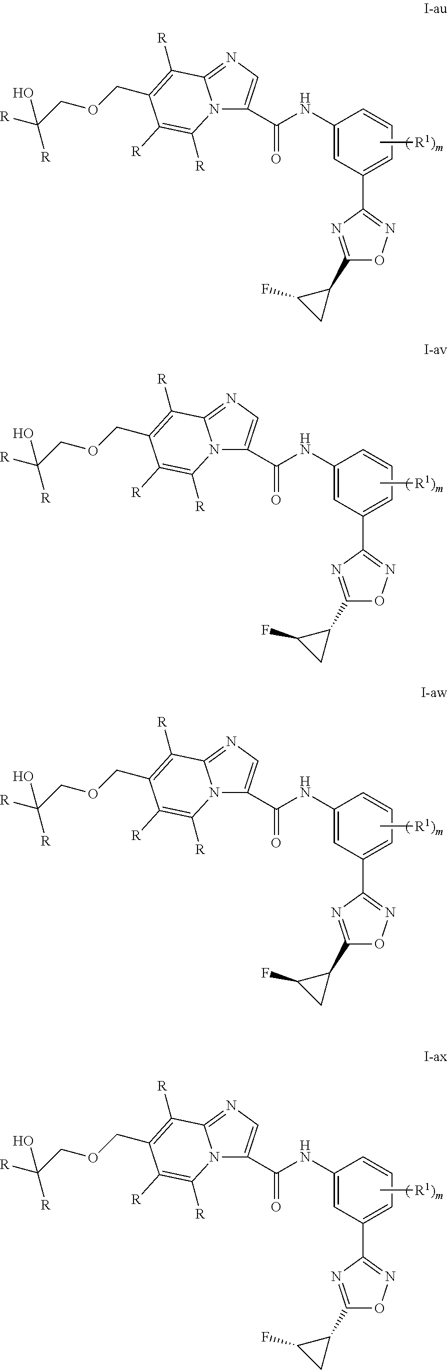

wherein R, R¹, and m are as defined above and described herein. In some embodiments, each R is independently selected from H, F, Cl, —CH₃, —CH₂F, —CHF₂ and —CF₃.

In some embodiments, a compound of the present disclosure is represented by any of the following or a pharmaceutically acceptable salt thereof:

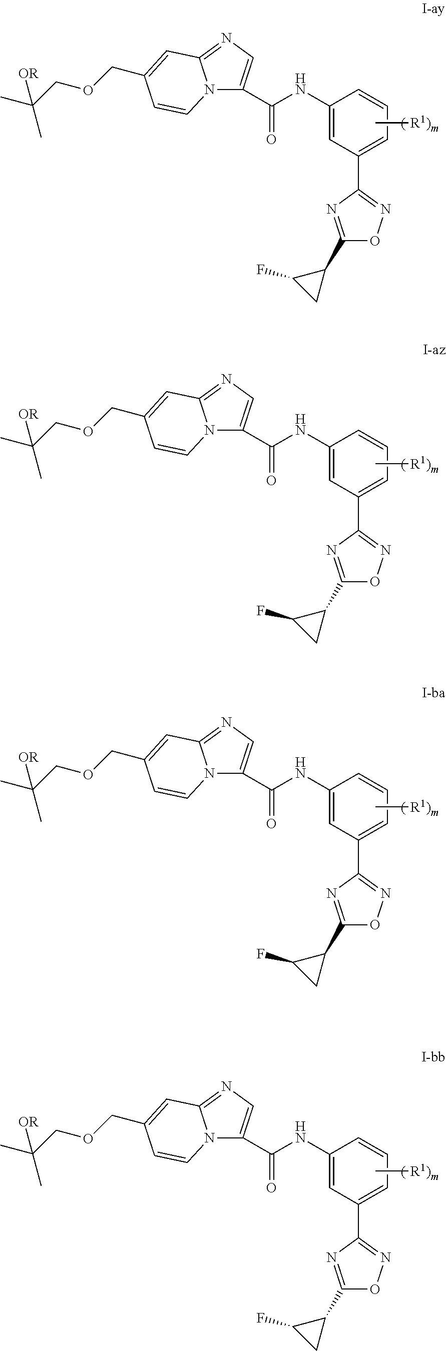

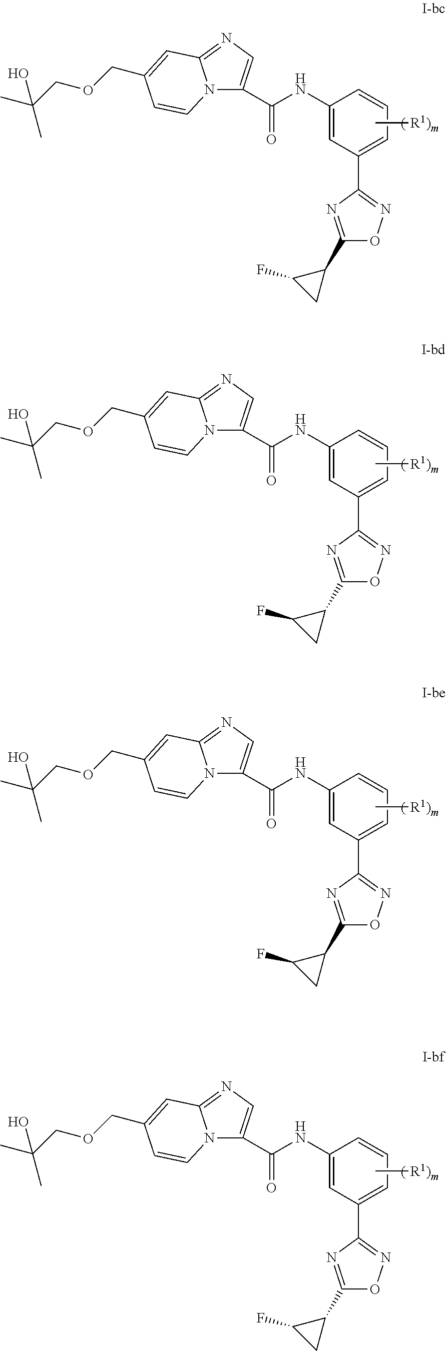

wherein R, R¹, and m are as defined above and described herein.

In some embodiments, a compound of the present disclosure is represented by any of the following or a pharmaceutically acceptable salt thereof:

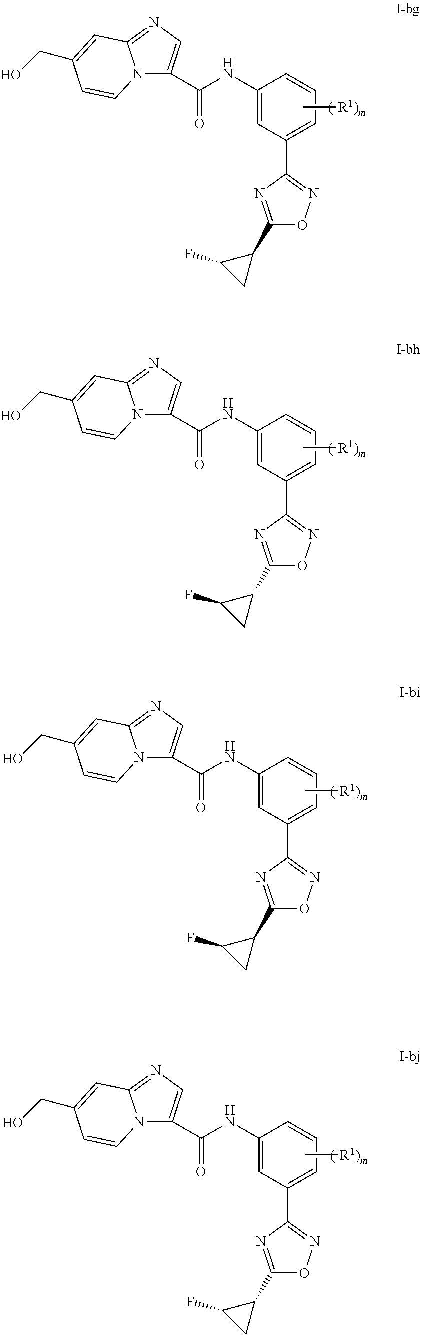

wherein R¹ and m are as defined above and described herein.

In some embodiments, a compound of the present disclosure is represented by any of the following or a pharmaceutically acceptable salt thereof:

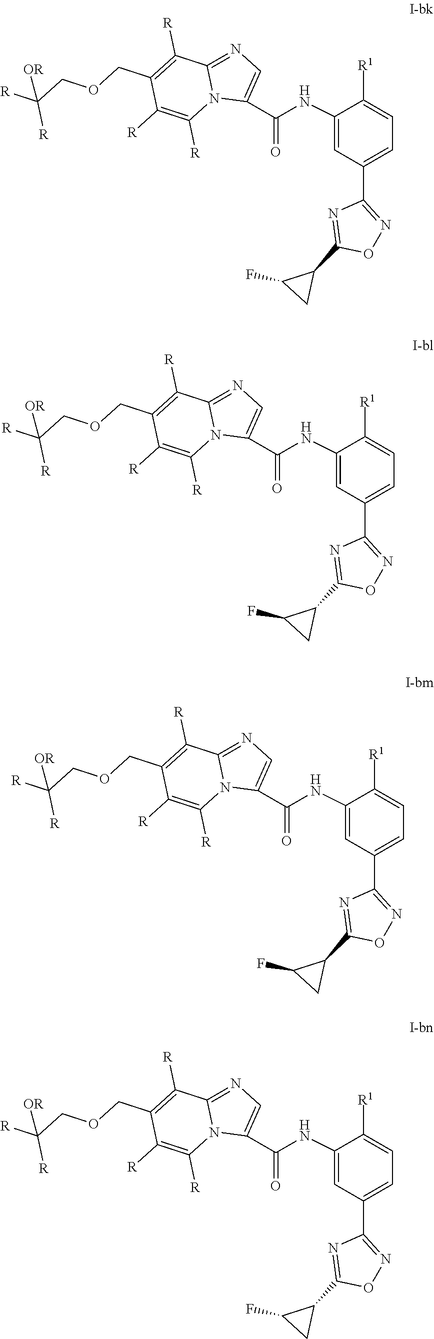

wherein R and R¹ are as defined above and described herein. In some embodiments, each R is independently selected from H, F, Cl, —CH₃, —CH₂F, —CHF₂ and —CF₃.

In some embodiments, a compound of the present disclosure is represented by any of the following or a pharmaceutically acceptable salt thereof:

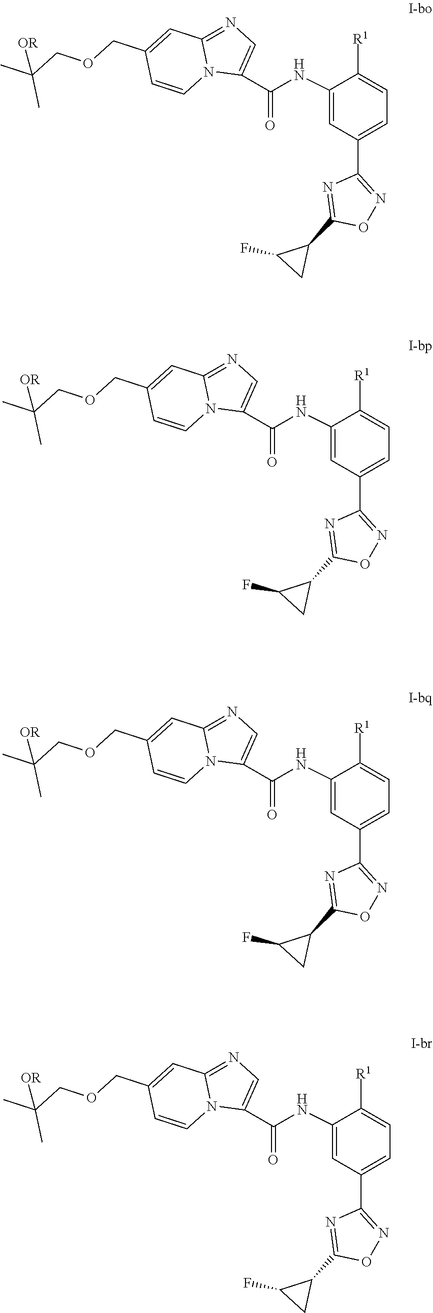

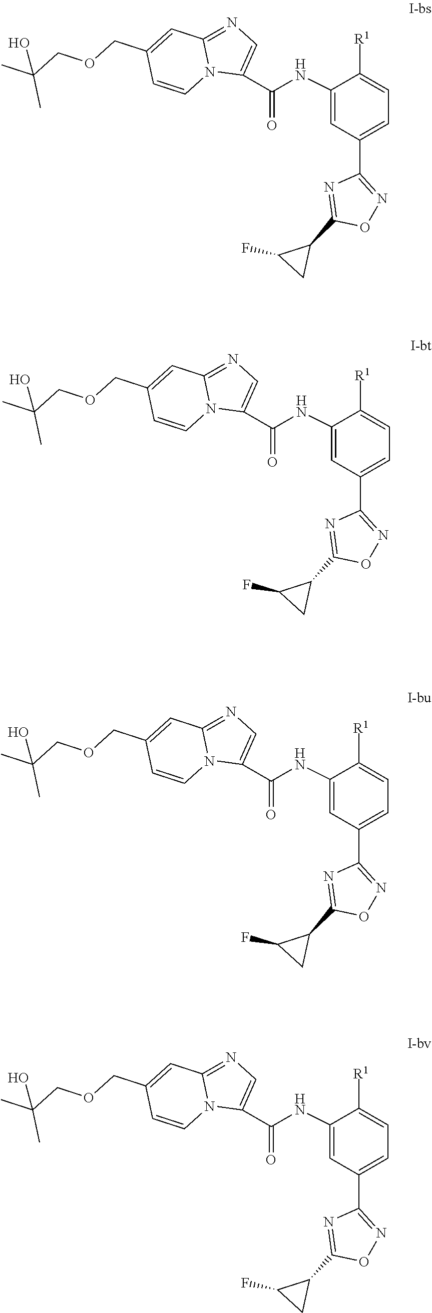

wherein R and R¹ are as defined above and described herein.

In some embodiments, a compound of the present disclosure is represented by any of the following or a pharmaceutically acceptable salt thereof:

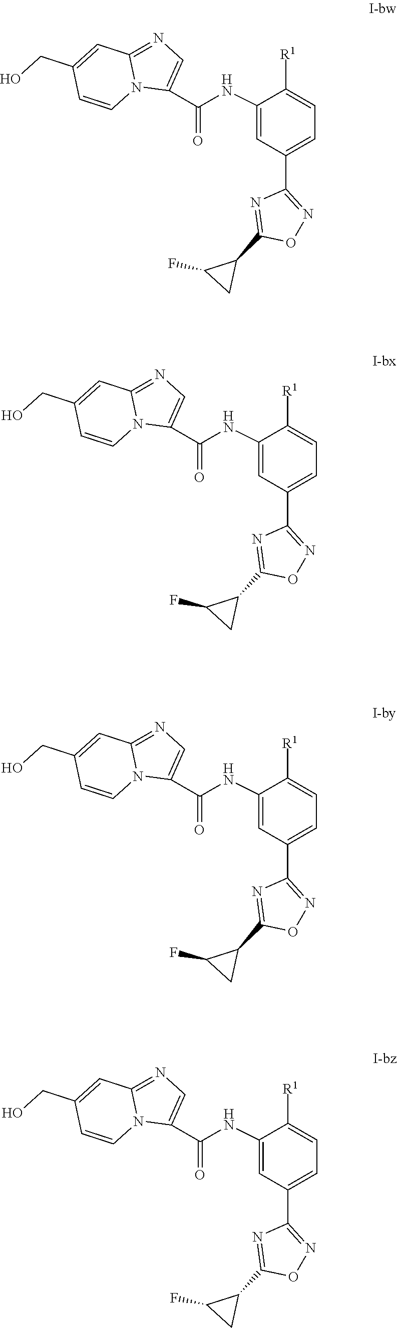

wherein R¹ is as defined above and described herein.

In some embodiments, a compound of the present disclosure is represented by either of the following or a pharmaceutically acceptable salt thereof:

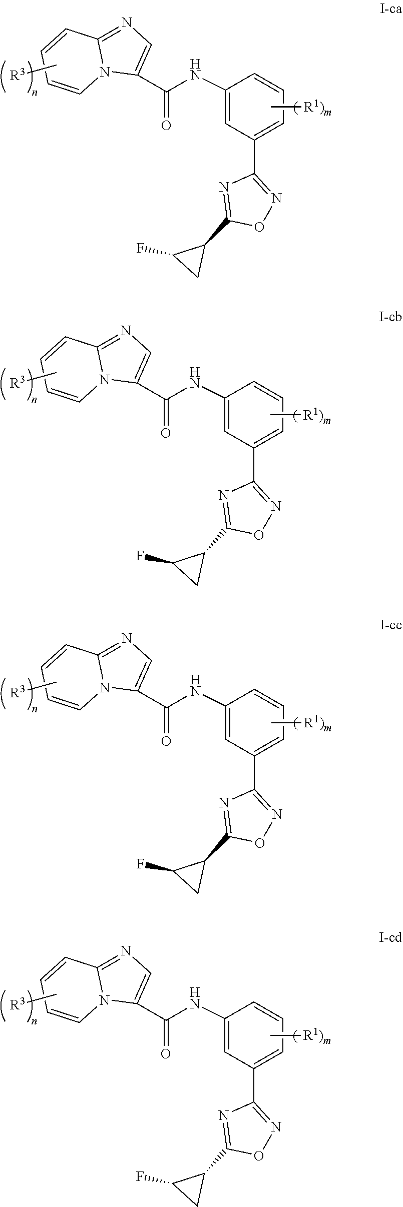

wherein R¹, R³, m and n are as defined above and described herein. In some such embodiments, n is 1 and R³ is H,

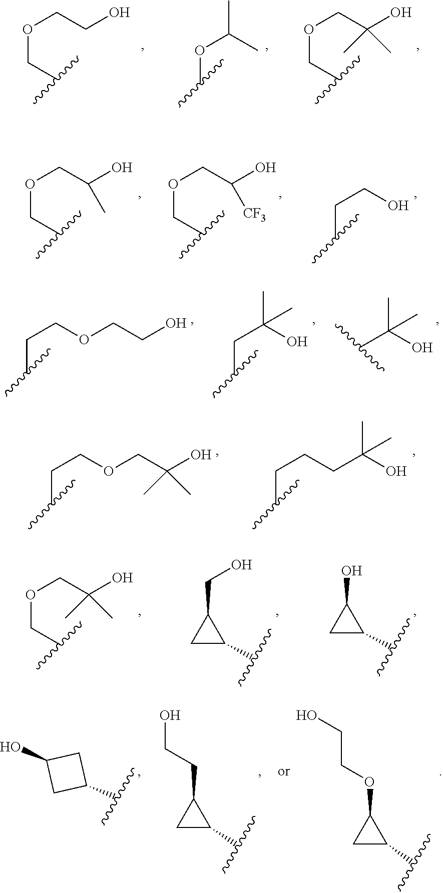

In some such embodiments n is 2 and each R³ is independently H, F, Cl

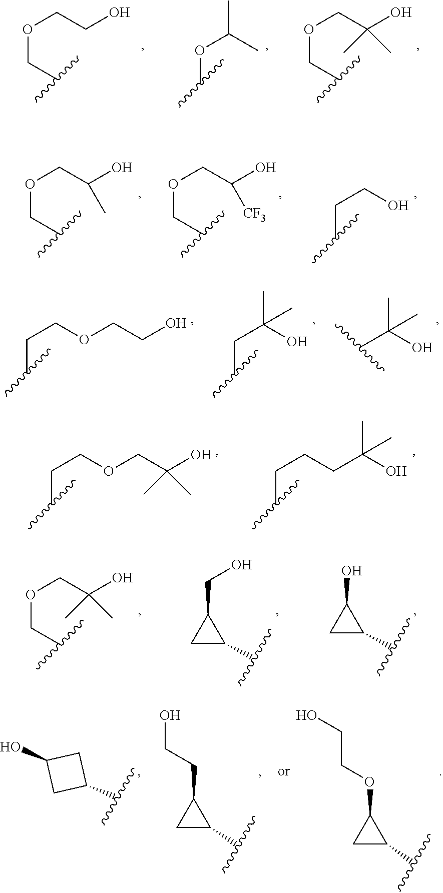

In some such embodiments n is 2, one of R³ is selected from H, F and Cl, and second R³ is selected from

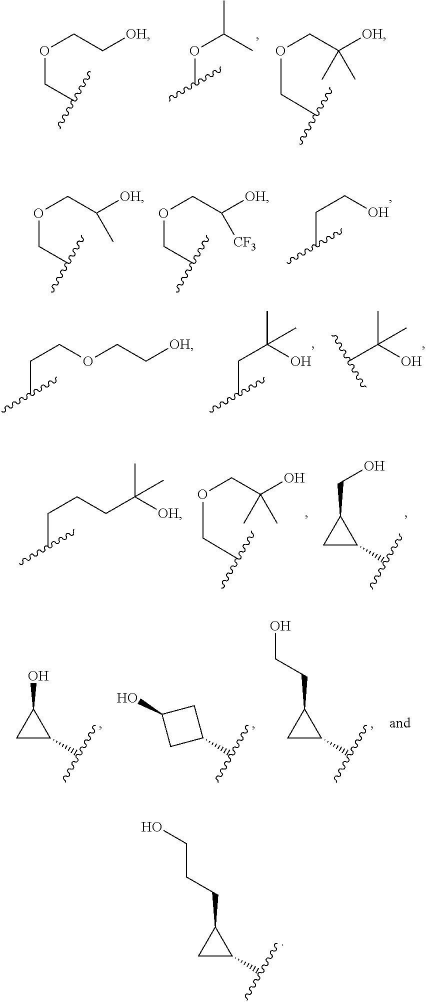

In some embodiments, a compound of the present disclosure is represented by either of the following or a pharmaceutically acceptable salt thereof:

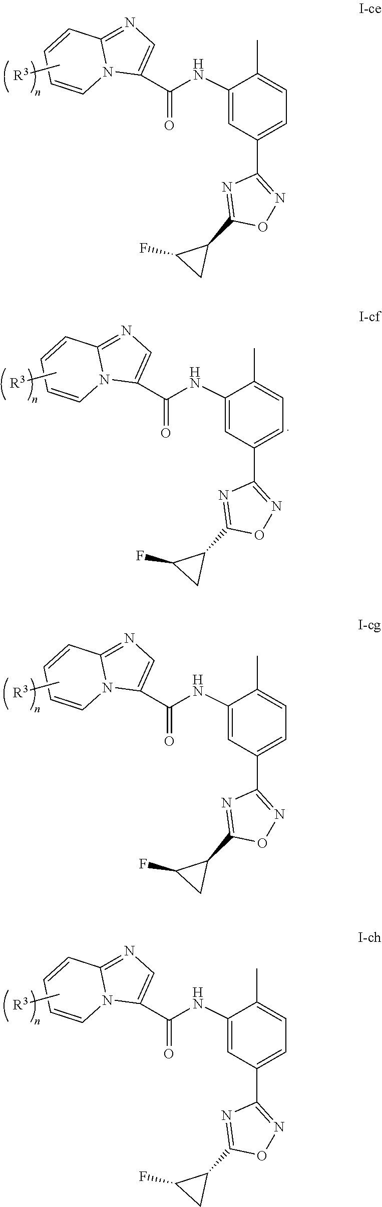

wherein R³ and n are as defined above and described herein. In some such embodiments, n is 1 and R³ is H,

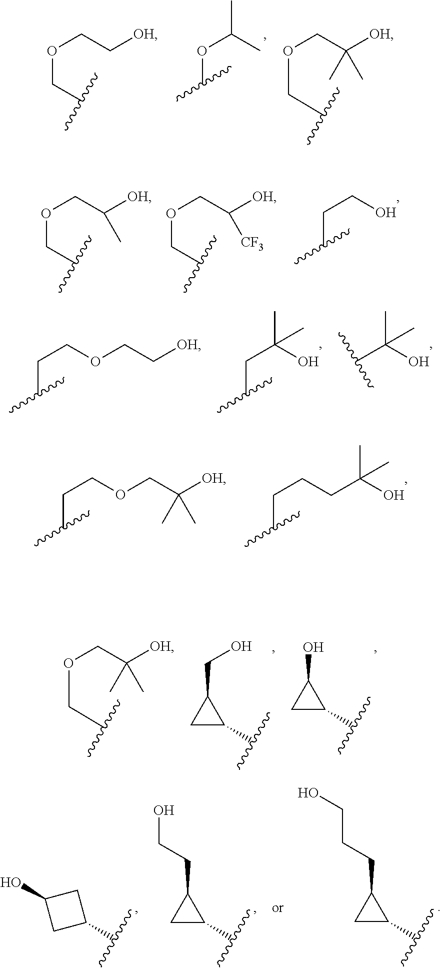

In some such embodiments, n is 2 and each R³ is independently H, F, Cl,

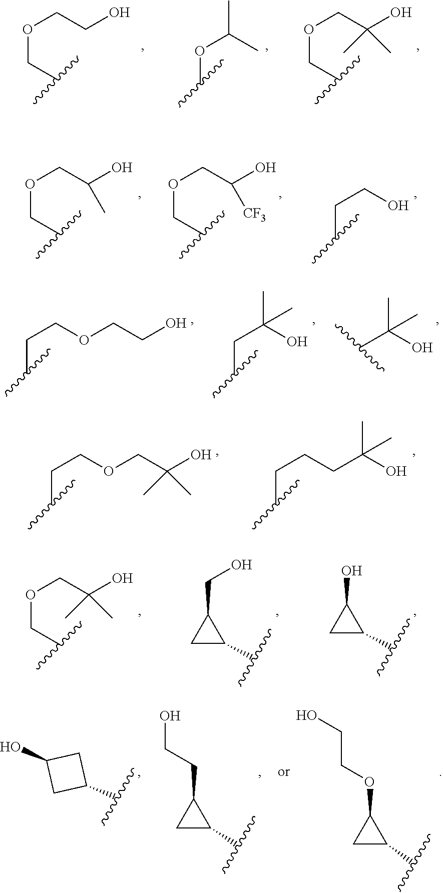

In some such embodiments, n is 2, one of R³ is selected from H, F and Cl, and second R³ is selected from

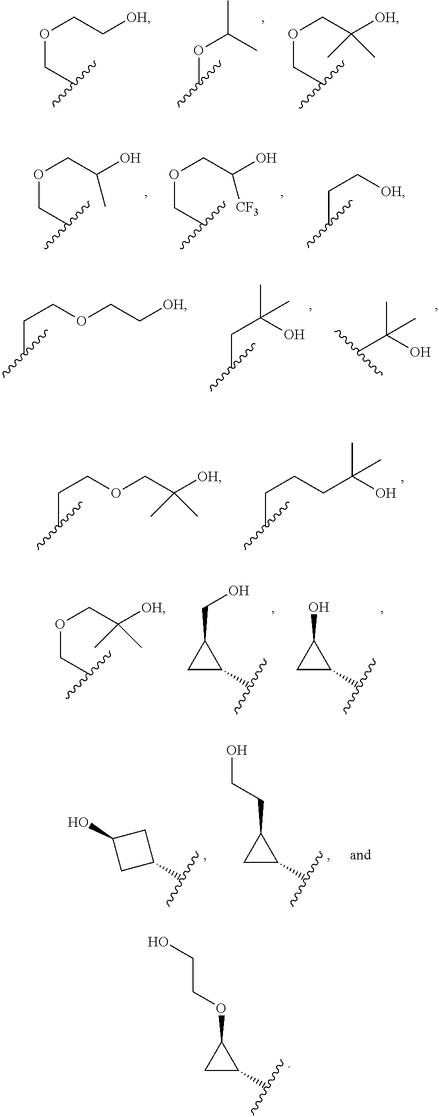

In some embodiments, a compound of the present disclosure is represented by any of the following or a pharmaceutically acceptable salt thereof:

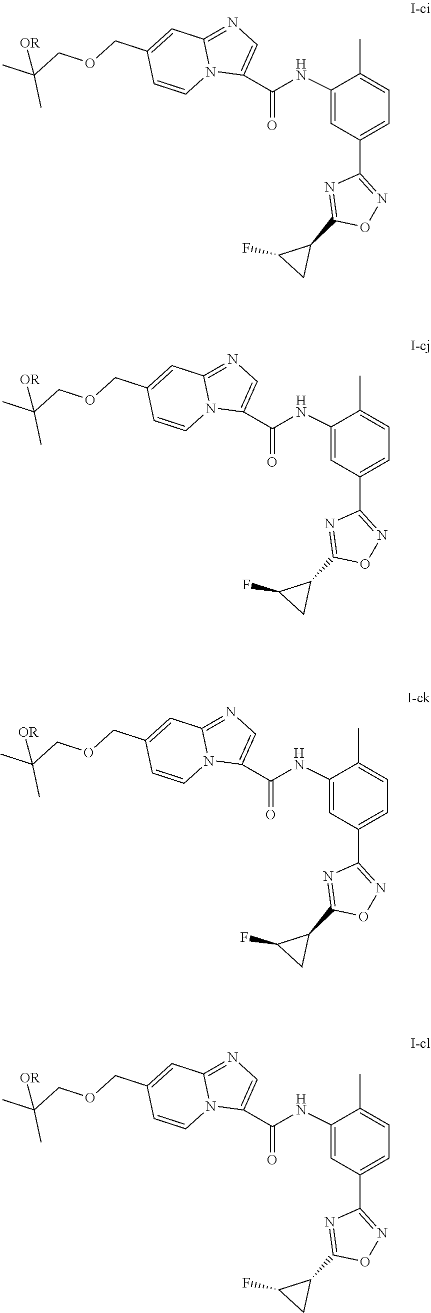

wherein R is as defined above and described herein.

In some embodiments, a compound of the present disclosure is represented by any of the following or a pharmaceutically acceptable salt thereof:

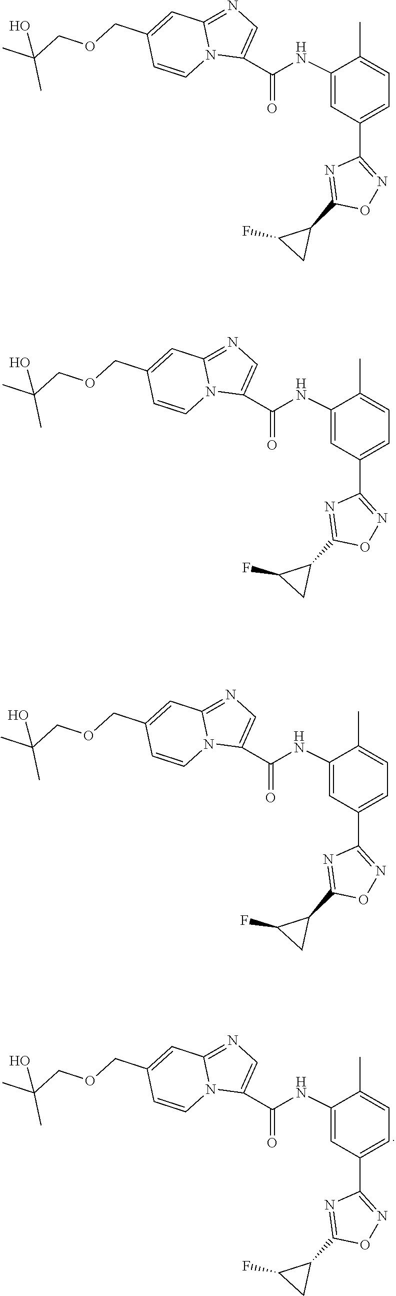 [SMILES: Cc1ccc(-c2noc([C@H]3C[C@@H]3F)n2)cc1NC(=O)c1cnc2cc(COCC(C)(C)O)ccn12 ; Cc1ccc(-c2noc([C@@H]3C[C@H]3F)n2)cc1NC(=O)c1cnc2cc(COCC(C)(C)O)ccn12 ; Cc1ccc(-c2noc([C@H]3C[C@H]3F)n2)cc1NC(=O)c1cnc2cc(COCC(C)(C)O)ccn12 ; Cc1ccc(-c2noc([C@@H]3C[C@@H]3F)n2)cc1NC(=O)c1cnc2cc(COCC(C)(C)O)ccn12]

In some embodiments, a compound of the present disclosure is represented by any of the following or a pharmaceutically acceptable salt thereof:

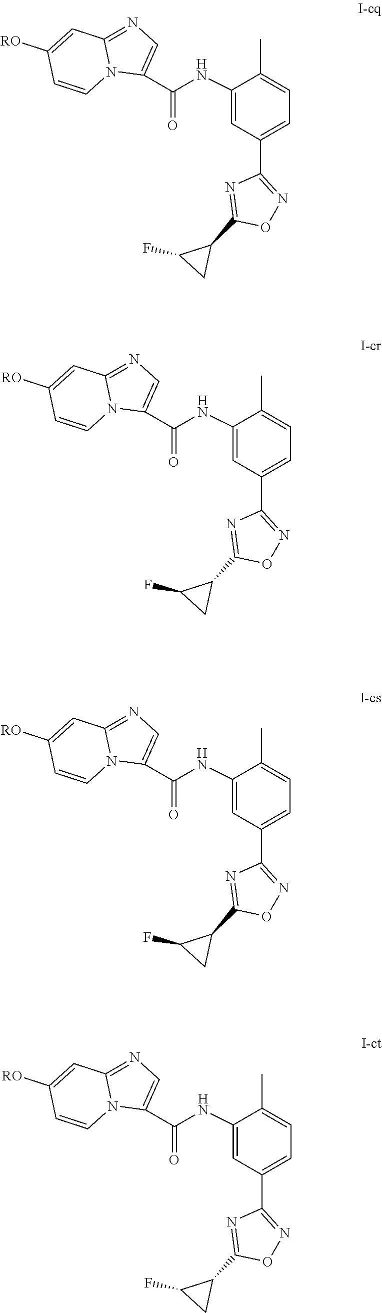

wherein R is as defined above and described herein. In some embodiments, R is i-Pr group.

In some embodiments, a compound of the present disclosure is represented by any of the following or a pharmaceutically acceptable salt thereof:

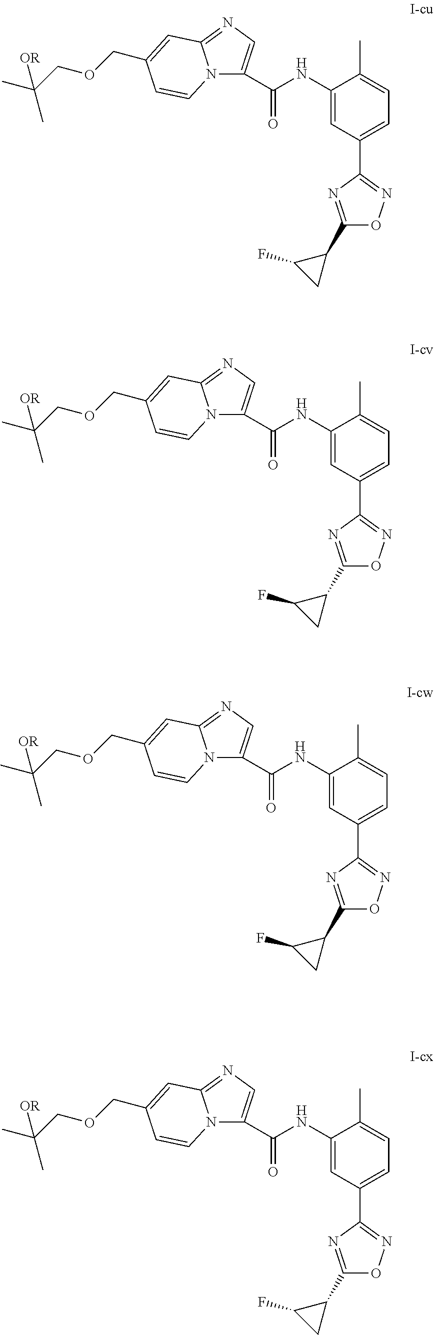

wherein R is as defined above and described herein.

In some embodiments, a compound of the present disclosure is represented by any of the following or a pharmaceutically acceptable salt thereof:

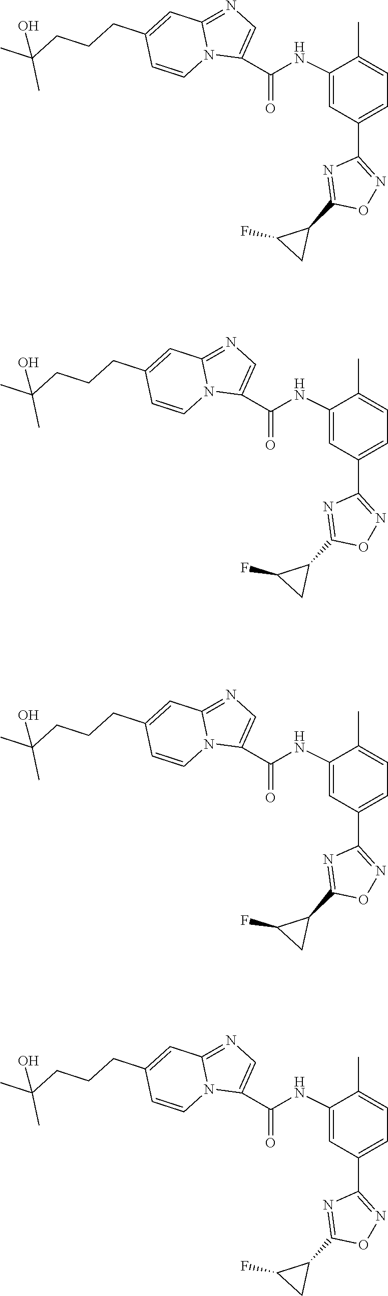 [SMILES: Cc1ccc(-c2noc([C@H]3C[C@@H]3F)n2)cc1NC(=O)c1cnc2cc(CCCC(C)(C)O)ccn12 ; Cc1ccc(-c2noc([C@@H]3C[C@H]3F)n2)cc1NC(=O)c1cnc2cc(CCCC(C)(C)O)ccn12 ; Cc1ccc(-c2noc([C@H]3C[C@H]3F)n2)cc1NC(=O)c1cnc2cc(CCCC(C)(C)O)ccn12 ; Cc1ccc(-c2noc([C@@H]3C[C@@H]3F)n2)cc1NC(=O)c1cnc2cc(CCCC(C)(C)O)ccn12]

In some embodiments, a compound of the present disclosure is represented by any of the following or a pharmaceutically acceptable salt thereof:

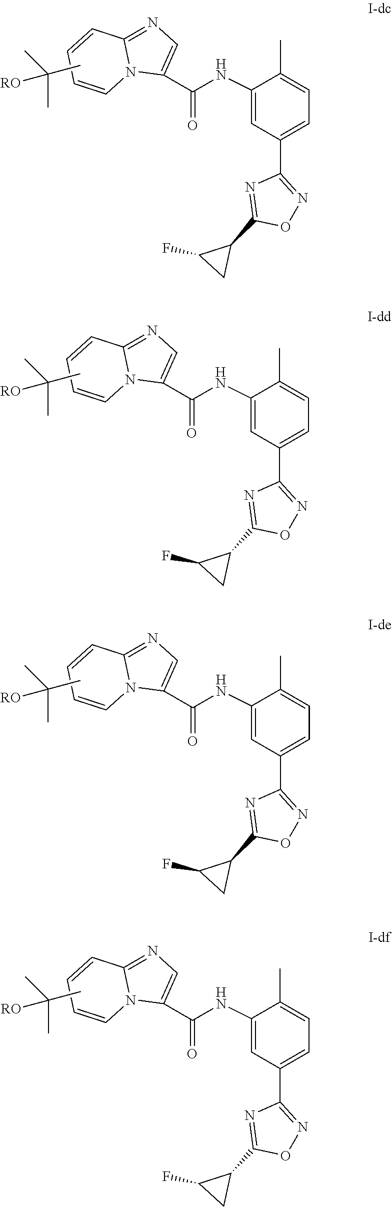

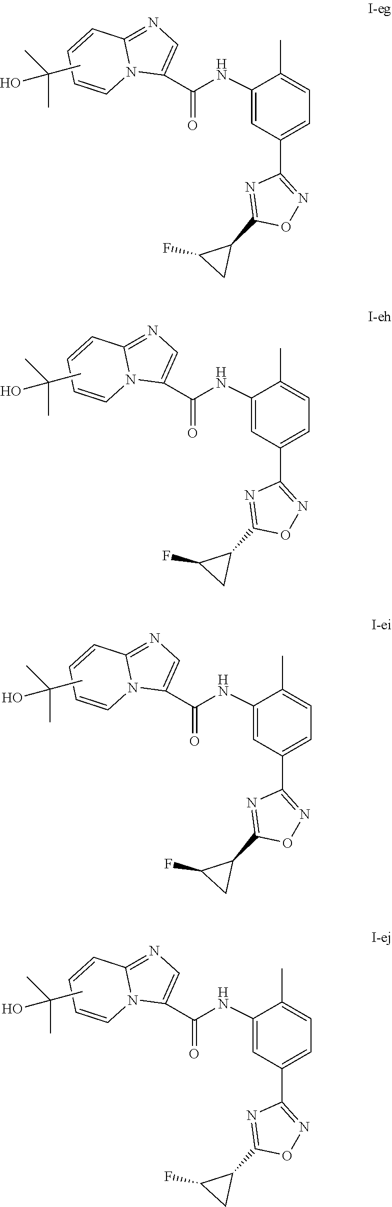

wherein R is as defined above and described herein.

In some embodiments, a compound of the present disclosure is represented by any of the following or a pharmaceutically acceptable salt thereof:

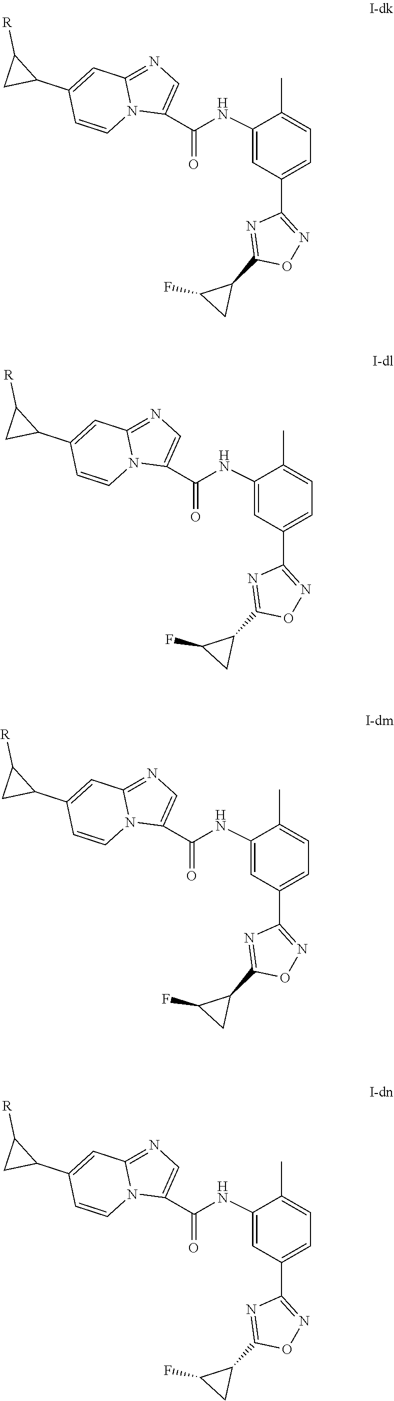

wherein R is as defined above and described herein. In some embodiments, R is —OH,

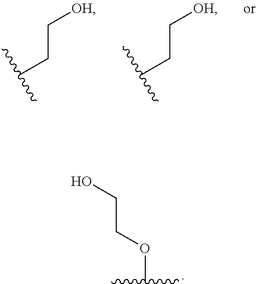

In some embodiments, a compound of the present disclosure is represented by any of the following or a pharmaceutically acceptable salt thereof:

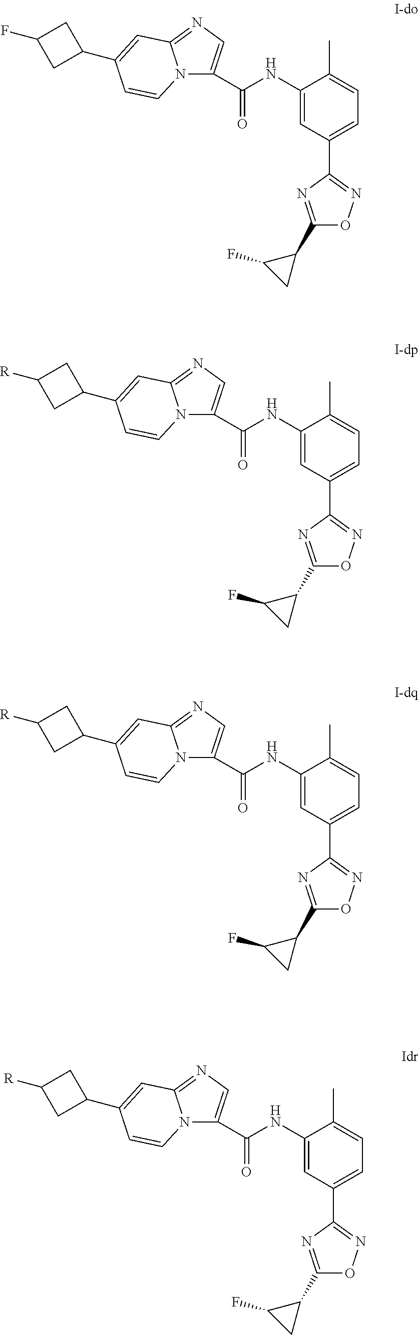

wherein R is as defined above and described herein. In some embodiments, R is —OH.

In some embodiments, a compound of the present disclosure is represented by any of the following or a pharmaceutically acceptable salt thereof:

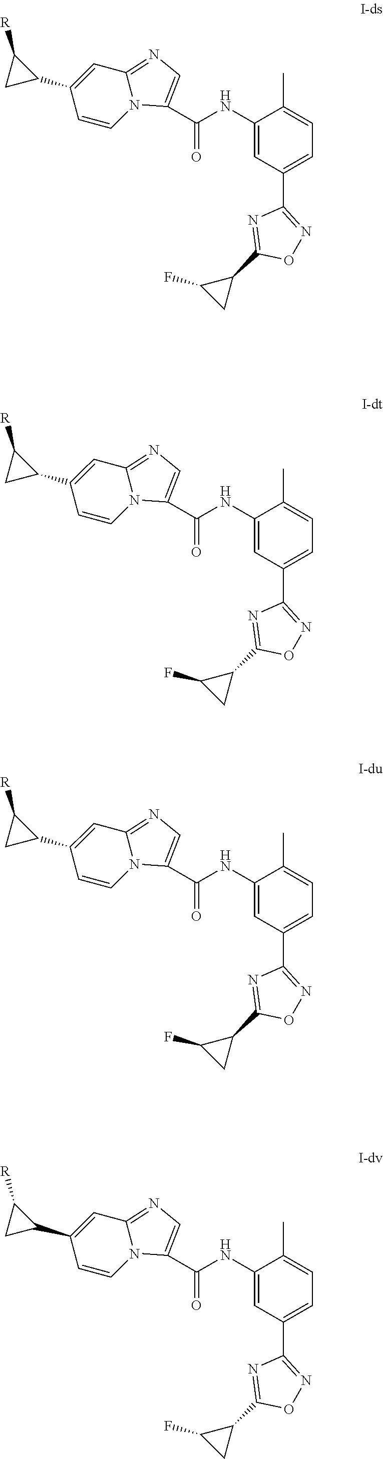

wherein R is as defined above and described herein. In some embodiments, R is —OH,

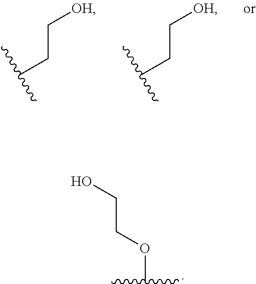

In some embodiments, a compound of the present disclosure is represented by any of the following or a pharmaceutically acceptable salt thereof:

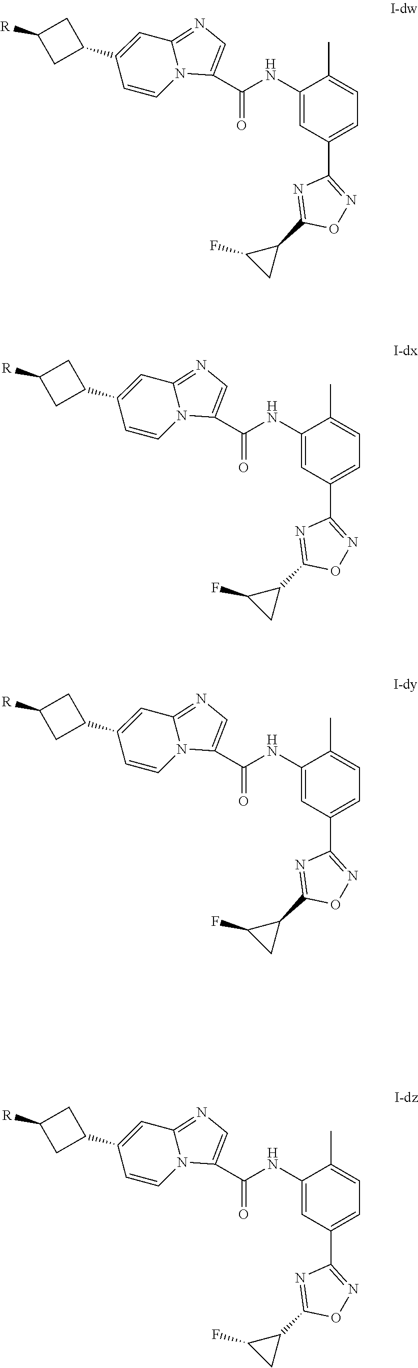

wherein R is as defined above and described herein. In some embodiments, R is —OH.

In some embodiments, a compound of the present disclosure is represented by any of the following or a pharmaceutically acceptable salt thereof:

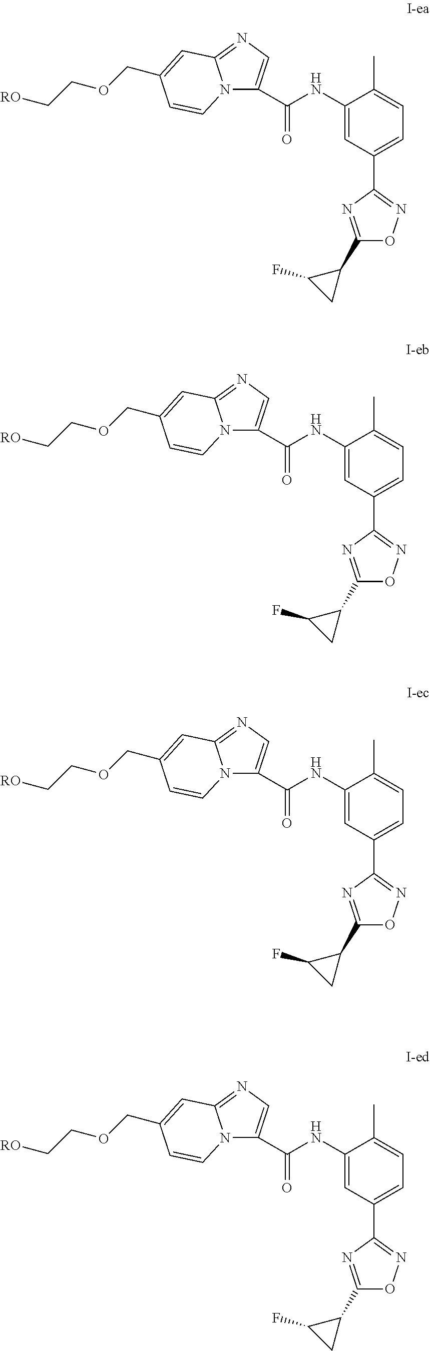

wherein R is as defined above and described herein.

In some embodiments, a compound of the present disclosure is represented by any of the following or a pharmaceutically acceptable salt thereof:

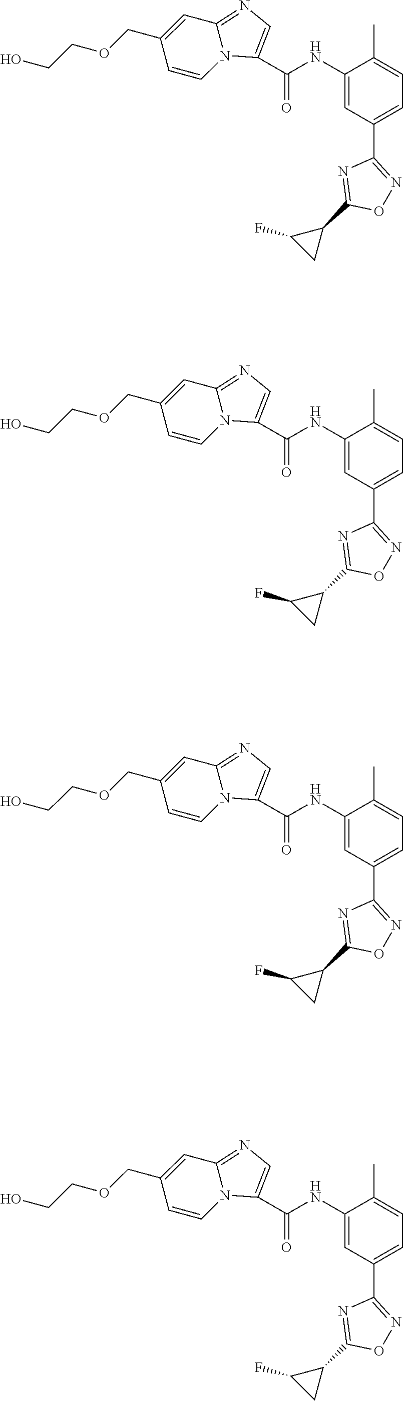 [SMILES: Cc1ccc(-c2noc([C@H]3C[C@@H]3F)n2)cc1NC(=O)c1cnc2cc(COCCO)ccn12 ; Cc1ccc(-c2noc([C@@H]3C[C@H]3F)n2)cc1NC(=O)c1cnc2cc(COCCO)ccn12 ; Cc1ccc(-c2noc([C@H]3C[C@H]3F)n2)cc1NC(=O)c1cnc2cc(COCCO)ccn12 ; Cc1ccc(-c2noc([C@@H]3C[C@@H]3F)n2)cc1NC(=O)c1cnc2cc(COCCO)ccn12]

In some embodiments, a compound of the present disclosure is represented by any of the following or a pharmaceutically acceptable salt thereof:

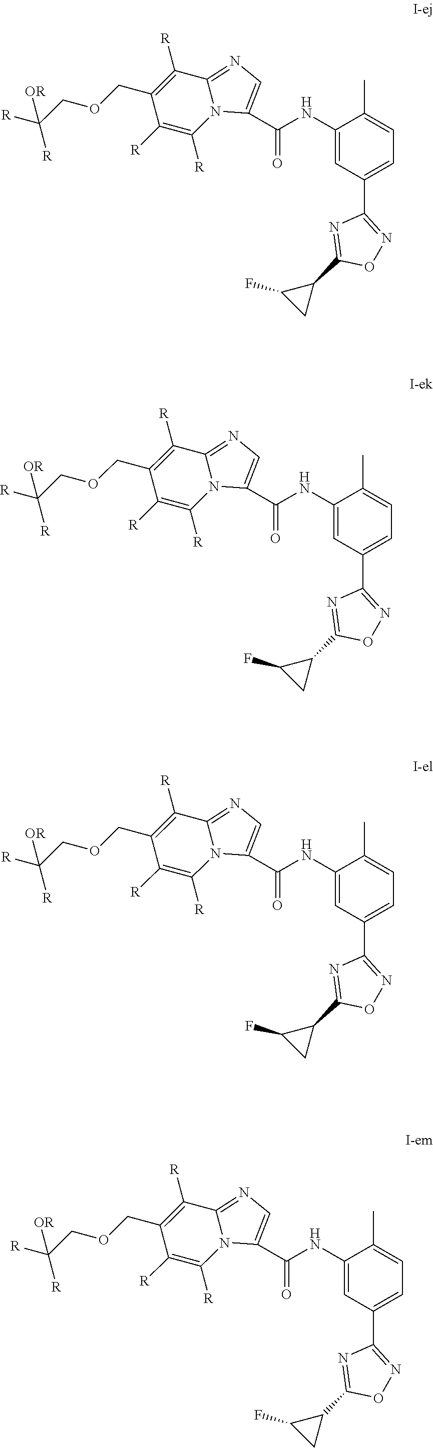

wherein R is as defined above and described herein. In some embodiments, each R is independently selected from H, F, Cl, —CH₃, —CH₂F, —CHF₂ and —CF₃.

In some embodiments, a compound of the present disclosure is represented by any of the following or a pharmaceutically acceptable salt thereof:

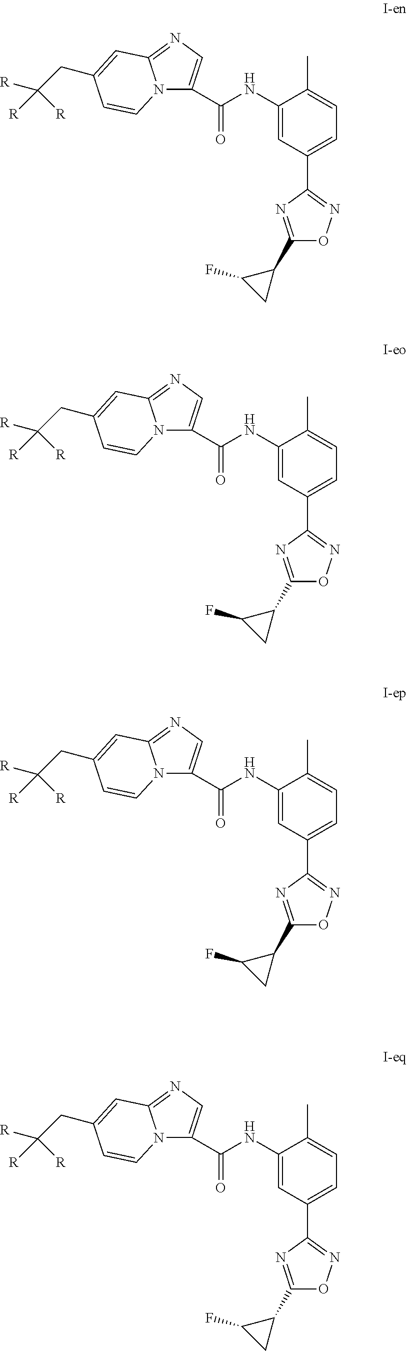

wherein R is as defined above and described herein. In some embodiments, each R is independently selected from H, —CH₃,

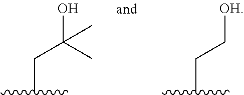

In some embodiments, a compound of the present disclosure is represented by any of the following or a pharmaceutically acceptable salt thereof:

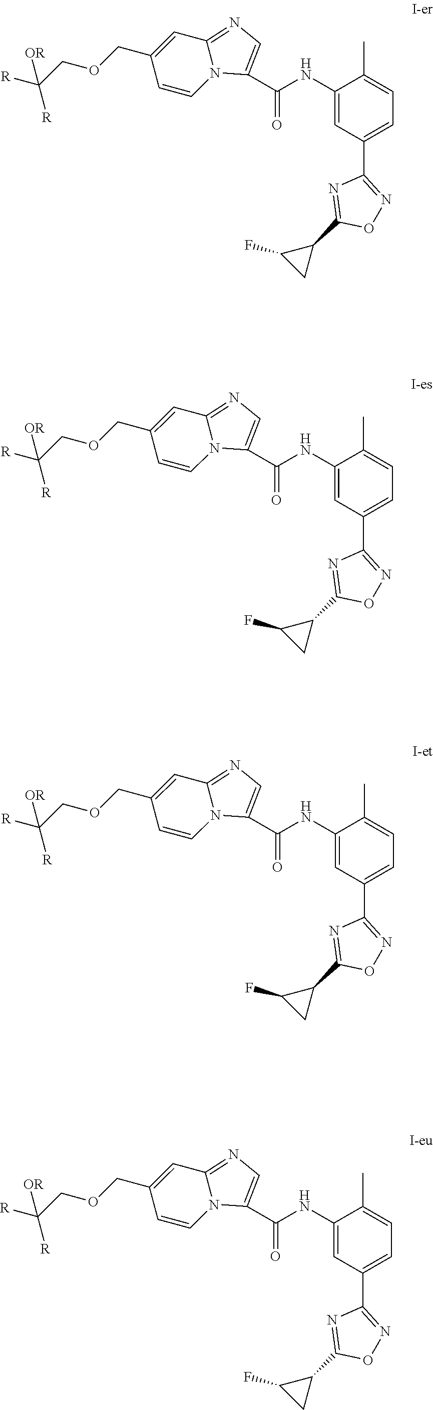

wherein R is as defined above and described herein. In some embodiments, each R is independently selected from H, —CH₃, —CH₂F, —CHF₂ and —CF₃.

In some embodiments, a compound of the present disclosure is represented by any of the following or a pharmaceutically acceptable salt thereof:

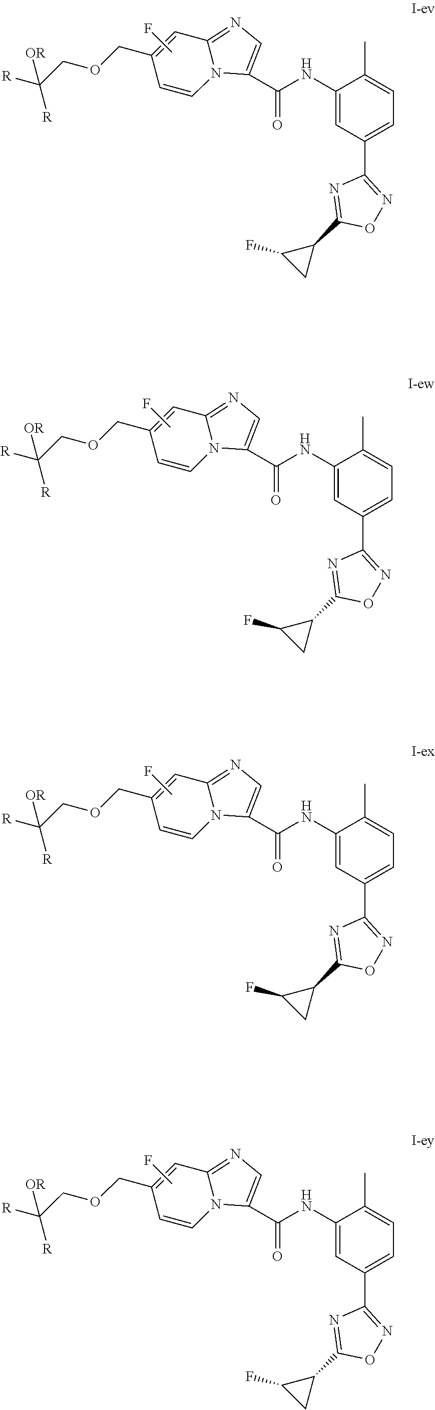

wherein R is as defined above and described herein. In some embodiments, each R is independently selected from H, —CH₃, —CH₂F, —CHF₂ and —CF₃.

In some embodiments, a compound of the present disclosure is represented by any of the following or a pharmaceutically acceptable salt thereof:

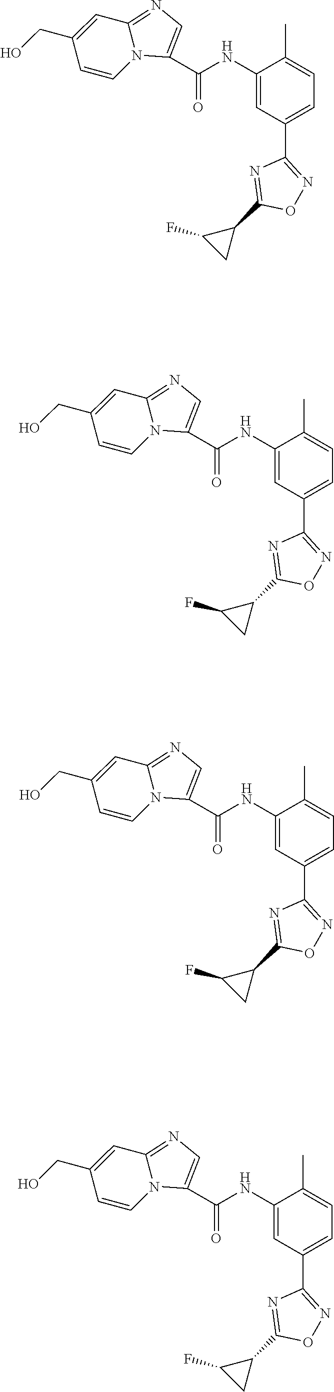 [SMILES: Cc1ccc(-c2noc([C@H]3C[C@@H]3F)n2)cc1NC(=O)c1cnc2cc(CO)ccn12 ; Cc1ccc(-c2noc([C@@H]3C[C@H]3F)n2)cc1NC(=O)c1cnc2cc(CO)ccn12 ; Cc1ccc(-c2noc([C@H]3C[C@H]3F)n2)cc1NC(=O)c1cnc2cc(CO)ccn12 ; Cc1ccc(-c2noc([C@@H]3C[C@@H]3F)n2)cc1NC(=O)c1cnc2cc(CO)ccn12]

In some embodiments, a compound of the present disclosure is represented by either of the following or a pharmaceutically acceptable salt thereof:

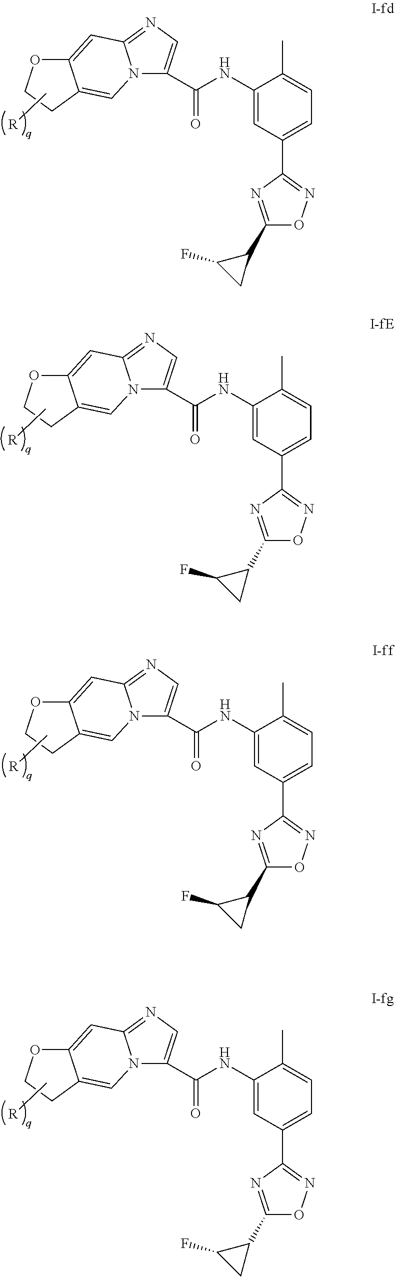

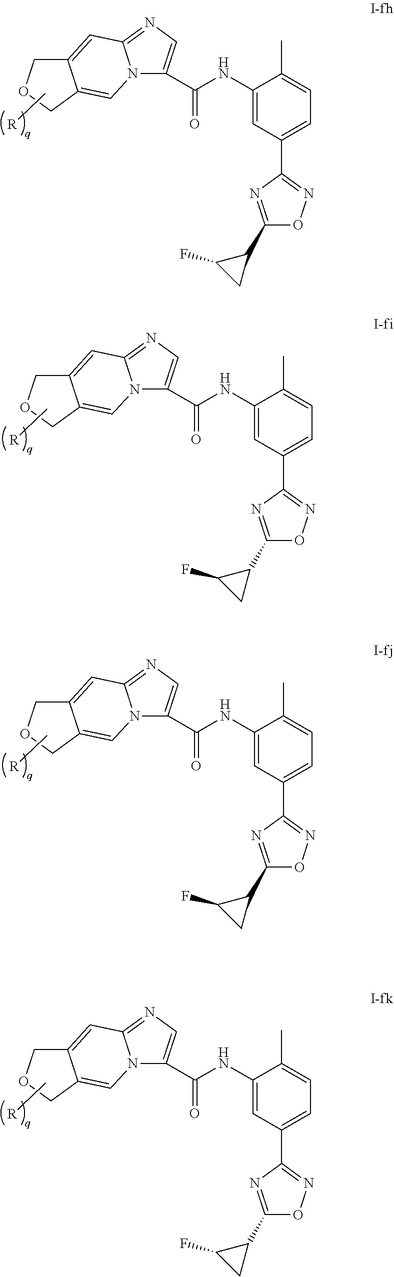

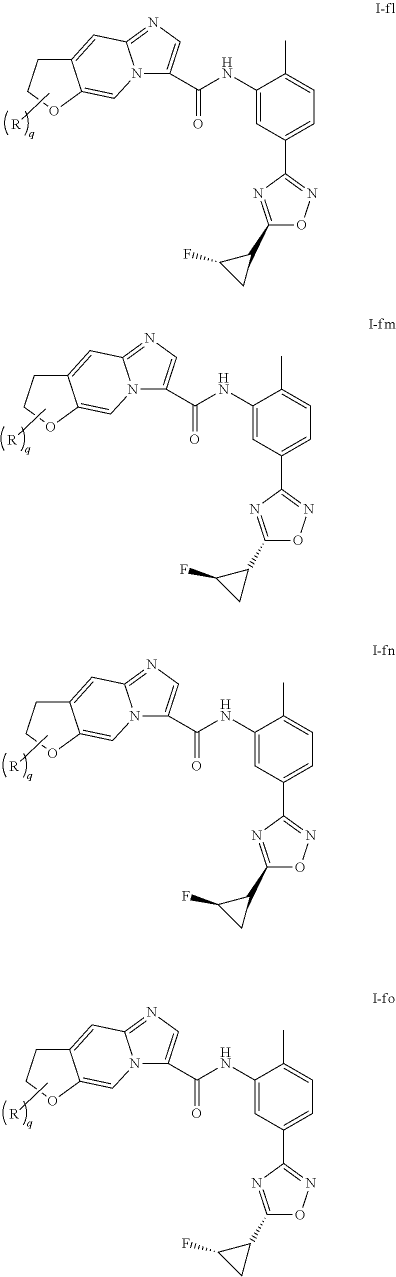

wherein R and q are as defined above and described herein. In some embodiments, q is 1. In some embodiments, q is 2. In some embodiments, R is Me. In some embodiments, the two R groups are present on the same carbon atom. In some embodiments, the two R groups are present on different carbon atoms. In some embodiments, R is Me, q is 2 and the two methyl groups are present on the same carbon atom.

In some embodiments, a compound of the present disclosure is represented by either of the following or a pharmaceutically acceptable salt thereof:

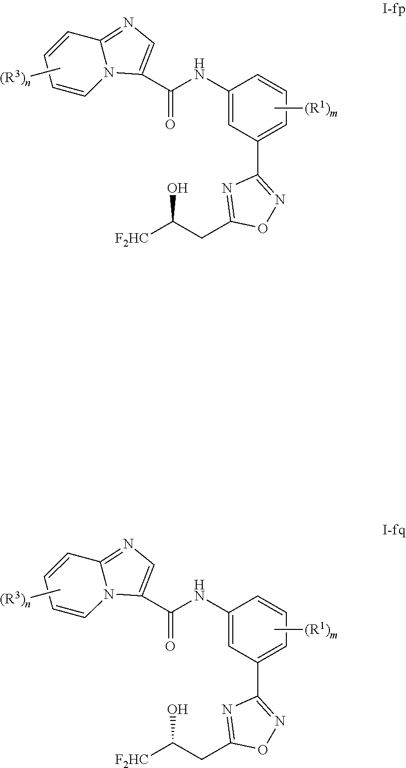

wherein R¹, R³, m and n are as defined above and described herein.

In some embodiments, a compound of the present disclosure is represented by either of the following or a pharmaceutically acceptable salt thereof:

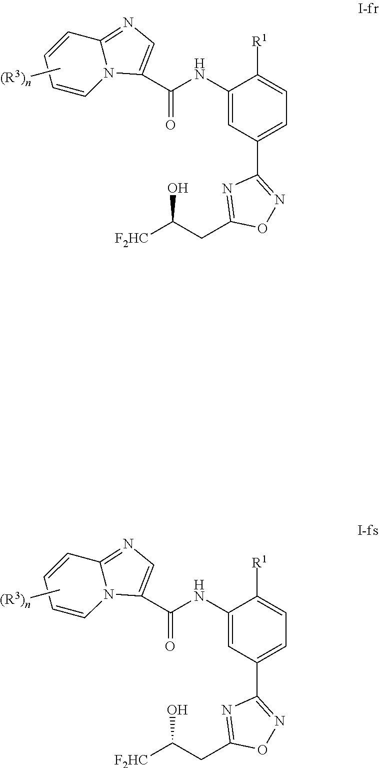

wherein R¹, R³, and n are as defined above and described herein.

In some embodiments, a compound of the present disclosure is represented by either of the following or a pharmaceutically acceptable salt thereof:

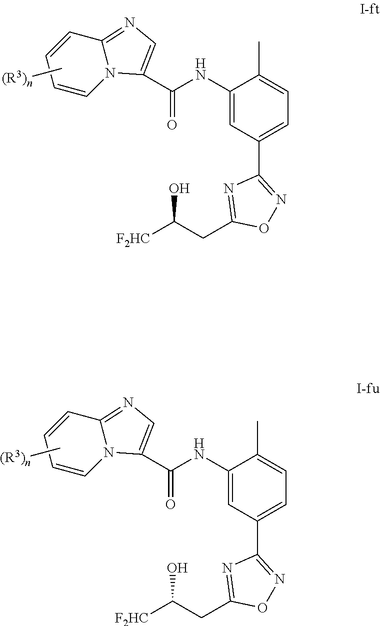

wherein R³ and n are as defined above and described herein. In some such embodiments, n is 0.

In some embodiments, a compound is of formulae I-a-I-fu above, wherein n is 0. In some embodiments, a compound is of formulae I-a-I-fu above, wherein n is 1.

In some embodiments, a compound is of formulae I-a-I-fu above, wherein n is 1 and R³ is selected from:

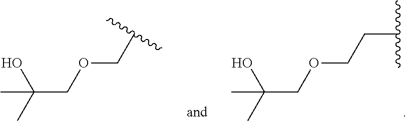

### Formula (I-1)

In some embodiments, the present disclosure provides a compound represented by Formula (I-1):

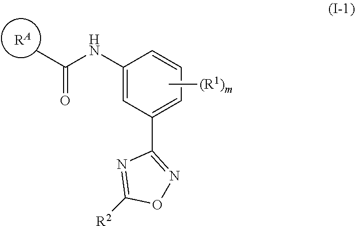

or a pharmaceutically acceptable salt thereof, wherein:
R¹ represents independently for each occurrence halogen, —CN, —OR, C₁₋₆ alkyl, or C₁₋₆ haloalkyl;
R² is C₁₋₆ aliphatic, phenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; or L¹-R⁴, wherein R² is substituted with p occurrences of R⁶;
L¹ is a C₁₋₂ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R⁴ is a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur;
R⁶ represents independently for each occurrence oxo, halogen, C₁₋₆ aliphatic, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or optionally substituted phenyl;
R^{A} is of any of the following structures:

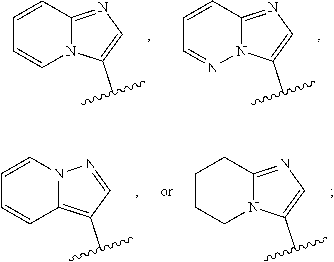 [SMILES: *c1cnc2ccccn12 ; *c1cnc2cccnn12 ; *c1cnn2ccccc12 ; *c1cnc2n1CCCC2]

each of which is substituted by n occurrences of R³;
R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, -L²-R⁵, or an optionally substituted group selected from C₁₋₆ aliphatic, C₁₋₆ haloaliphatic, phenyl, naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; each of which is substituted with r instances of R; or:
two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur;
L² represents independently for each occurrence a C₁₋₆ bivalent saturated or unsaturated, straight or branched hydrocarbon chain wherein one, two, or three methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂— or -Cy-; Cy represents independently for each occurrence phenyl, a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
R⁵ represents independently for each occurrence hydrogen, OR, C₁₋₆ aliphatic, C₁₋₆ haloaliphatic, or phenyl fused to a 5-6 membered saturated heterocyclic ring having 1-2 heteroatoms independently selected from nitrogen, oxygen, and sulfur;
each R is independently hydrogen, —CN, halogen, or an optionally substituted group selected from C₁₋₆ aliphatic; C₁₋₆ haloaliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur;
m is 0, 1, 2, 3, or 4;
n is 0, 1, 2, 3, 4, or 5;
p is 0, 1, 2, 3, 4, or 5; and
r is 0, 1, 2, 3, 4, or 5.

The definitions of variables in Formula I-1 above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-1.

The description above describes multiple embodiments relating to compounds of Formula I-1. The patent application specifically contemplates all combinations of the embodiments.

It has been surprisingly discovered that certain compounds of the present invention do not significantly penetrate the brain or minimally penetrate the brain, wherein the extent of brain penetration is measured by measuring “Kₚ,” i.e., the ratio of compound concentration in the brain and blood plasma (C_{brain}/Cₚₗₐₛₘₐ) as demonstrated by certain assays described herein. See, e.g., Example 100. Exemplary such compounds include, e.g., I-296. In some such embodiments, a compound of the present invention is characterized as having a Kₚ (brain) of less than about 0.7, less than about 0.6, less than about 0.5, less than about 0.4, less than about 0.3, less than about 0.2, or less than about 0.1. In some embodiments, a compound of the present invention is characterized as having a Kₚ of less than about 0.7. In some embodiments, a compound of the present invention is characterized by having a Kₚ of less than about 0.6. In some embodiments, a compound of the present invention is characterized by having a Kₚ of less than about 0.5. In some embodiments, a compound of the present invention is characterized by having a Kₚ of less than about 0.4. In some embodiments, a compound of the present invention is characterized by having a Kₚ of less than about 0.3. In some embodiments, a compound of the present invention is characterized by having a Kₚ of less than about 0.2. In some embodiments, a compound of the present invention is characterized by having a Kₚ of less than about 0.1. In some embodiments, a compound of the present invention is characterized by having a Kₚ of less than about 0.09. In some embodiments, a compound of the present invention is characterized by having a Kₚ of less than about 0.08. In some embodiments, a compound of the present invention is characterized by having a Kₚ of less than about 0.05. In some embodiments, a compound of the present invention is characterized by having a Kₚ of less than about 0.04. In some embodiments, a compound of the present invention is characterized by having a Kₚ of less than about 0.03. In some embodiments, a compound of the present invention is characterized by having a Kₚ of less than about 0.02. In some embodiments, a compound of the present invention is characterized by having a Kₚ of less than about 0.01. Various methods of assessing brain exposure are known to those of skill in the art and/or are described herein. Exemplary such compounds which do not significantly penetrate the brain or minimally penetrate the brain, as indicated by Kₚ, include, but are not limited to, I-114, I-71, I-84, I-114, and I-296.

In some embodiments, for compounds with low Kₚ values, determination of Kₚᵤᵤ from the unbound compounds plasma, brain and testes concentrations also supports peripheral restriction of the compounds.

In some embodiments, it has been surprisingly discovered that compounds of the present invention are Breast Cancer Resistance Protein (BCRP) efflux substrates. The human breast cancer resistance protein (BCRP, gene symbol ABCG2) is an ATP-binding cassette (ABC) efflux transporter. Among normal human tissues, BCRP is highly expressed on the apical membranes of the placental syncytiotrophoblasts, the intestinal epithelium, the liver hepatocytes, the endothelial cells of brain microvessels, testis, and the renal proximal tubular cells, contributing to the absorption, distribution, and elimination of drugs and endogenous compounds as well as tissue protection against xenobiotic exposure. As a result, BCRP has now been recognized by the FDA to be one of the key drug transporters involved in clinically relevant drug disposition.

Various methods of assessing whether a compound is a BCRP efflux substrate are known to those of skill in the art and/or are described herein, for instance in Example 99. Exemplary such compounds include, e.g., I-296. In some embodiments, a compound of the present invention exhibits an efflux ratio (ER) of about 1-fold, indicating substantially no efflux. In some embodiments, a compound of the present invention exhibits an efflux ratio (ER) of at least about 1.5-fold. In some embodiments, a compound of the present invention exhibits an efflux ratio (ER) of at least about 2.0-fold. In some embodiments, a compound of the present invention exhibits an efflux ratio (ER) of at least about 3.5-fold. In some embodiments, a compound of the present invention exhibits an efflux ratio (ER) of at least about 4.0-fold. In some embodiments, a compound of the present invention exhibits an efflux ratio (ER) of at least about 4.5-fold. In some embodiments, a compound of the present invention exhibits an efflux ratio (ER) of at least about 5-fold. In some embodiments, a compound of the present invention exhibits an efflux ratio (ER) of at least about 6-fold. In some embodiments, a compound of the present invention exhibits an efflux ratio (ER) of at least about 7-fold. In some embodiments, a compound of the present invention exhibits an efflux ratio (ER) of at least about 8-fold. In some embodiments, a compound of the present invention exhibits an efflux ratio (ER) of at least about 9-fold. In some embodiments, a compound of the present invention exhibits an efflux ratio (ER) of at least about 10-fold. In some embodiments, a compound of the present invention exhibits an efflux ratio (ER) of at least about 15-fold. In some embodiments, a compound of the present invention exhibits an efflux ratio (ER) of at least about 20-fold. In some embodiments, a compound of the present invention exhibits an efflux ratio (ER) of at least about 25-fold. In some embodiments, a compound of the present invention exhibits an efflux ratio (ER) of at least about 30-fold. In some embodiments, a compound of the present invention exhibits an efflux ratio (ER) of at least about 35-fold. In some embodiments, a compound of the present invention exhibits an efflux ratio (ER) of at least about 40-fold. In some embodiments, a compound of the present invention exhibits an efflux ratio (ER) of at least about 45-fold. In some embodiments, a compound of the present invention exhibits an efflux ratio (ER) of at least about 50-fold.

In some embodiments, it has been surprisingly discovered that certain compounds of the present invention do not significantly inhibit BCRP.

Various methods of assessing whether a compound is a BCRP inhibitor are known to those of skill in the art and/or are described herein, for instance in Example 103. Exemplary such compounds include, e.g., I-296. In some embodiments, compounds of the present invention have a BCRP inhibition IC₅₀ of about 400 nM, or about 500 nM, or about 600 nM, or about 700 nM, or about 800 nM, or about 900 nM. In some embodiments, compounds of the present invention have a BCRP inhibition IC₅₀ of about 1 mM, about 2 mM, about 3 mM, about 4 mM, about 5 mM, about 6 mM, about 7 mM, about 8 mM, about 9 mM, or about 10 mM. In some embodiments, compounds of the present invention have a BCRP inhibition IC₅₀ of between about 500 nM and 10 mM. In some embodiments, compounds of the present invention have a BCRP inhibition IC₅₀ of between about 500 nM and 5 mM. In some embodiments, compounds of the present invention have a BCRP inhibition IC₅₀ of between about 500 nM and 1 mM. In some embodiments, compounds of the present invention have a BCRP inhibition IC₅₀ of between about 1 mM and 10 mM. In some embodiments, compounds of the present invention have a BCRP inhibition IC₅₀ of between about 1 mM and 5 mM. In some embodiments, compounds of the present invention have a BCRP inhibition IC₅₀ of between about 5 mM and 10 mM. Exemplary compounds of the present invention are described further herein.

In some embodiments, it has been surprising discovered that compounds of the present invention are P-glycoprotein (PGP) efflux substrates. P-glycoprotein (PGP), an efflux membrane transporter, is also referred to in the art as multi-drug resistance protein 1 ((MDR1), permeability glycoprotein, P-gp, or Pgp, encoded by MDR1/ABCB1 and belonging to the family of ATP-binding cassette transporters), and is widely distributed throughout the body and responsible for limiting cellular uptake and the distribution of xenobiotics and toxic substances. PGP is one of the most important transporters at the blood-brain barrier (BBB), where it is highly expressed in the vessel walls of the brain capillaries functioning as an efflux pump. PGP is also located throughout the human body in organs or tissues with an excretory and/or barrier function, such as the liver, kidney, placenta, and testes.

With respect to the placenta, PGP has been found to have a role in the regulation of drug disposition to the fetus and has been extensively studied. Expression of PGP in the placental trophoblast layer has been confirmed at the mRNA and protein levels in all phases of pregnancy. Several in vitro and in vivo studies have demonstrated functional activity of the transporter in materno-fetal drug transport. PGP is able to actively pump drugs and other xenobiotics from trophoblast cells back to the maternal circulation, thus providing protection to the fetus.

In some embodiments, compounds of the present invention are efflux substrates of BRCP. In some embodiments, compounds of the present invention are efflux substrates of PGP. In some embodiments, compounds of the present invention are efflux substrates of one or both of BCRP and PGP.

It has been further surprisingly discovered that certain compounds of the present invention afford lower testes exposure, which may lead to better spermatogonia survival and/or spermatogonia maturation. Various methods of assessing whether a compound affords lower testes exposure are known to those of skill in the art and/or are described herein, for instance in Example 103. Exemplary such compounds include, e.g., I-296. Kₚ (testes) is defined as the ratio of compound concentration in the testes and in the plasma (Cₜₑₛₜₑₛ/Cₚₗₐₛₘₐ). By lower testes exposure is meant a compound measured as having a Kₚ (testes) of less than about 1.0, or less than about 0.9, or less than about 0.8, or less than about 0.7, or less than about 0.6, or less than about 0.5, or less than about 0.4, or less than about 0.3, or less than about 0.2, or less than about 0.1, or less than about 0.09, or less than about 0.08, or less than about 0.07, or less than about 0.06, or less than about 0.05, or less than about 0.04, or 0.03, or less than about 0.02, or less than about 0.01.

In some embodiments, a compound of the present invention is not an inducer of CYP3A4, as measured by CYP3A4 gene expression, for instance, see Example 101. In some embodiments, such may lead to reduced risk for drug-drug interactions. Exemplary such compounds include, e.g., I-296. For instance, in some embodiments, a compound of the present invention when measured as described herein exhibits an induction fold of less than or equal to about 10-fold. In some embodiments, a compound of the present invention when measured as described herein exhibits an induction fold of less than or equal to about 9-fold. In some embodiments, a compound of the present invention when measured as described herein exhibits an induction fold of less than or equal to about 8-fold. In some embodiments, a compound of the present invention when measured as described herein exhibits an induction fold of less than or equal to about 7-fold. In some embodiments, a compound of the present invention when measured as described herein exhibits an induction fold of less than or equal to about 6-fold. In some embodiments, a compound of the present invention when measured as described herein exhibits an induction fold of less than or equal to about 5-fold. In some embodiments, a compound of the present invention when measured as described herein exhibits an induction fold of less than or equal to about 4-fold. In some embodiments, a compound of the present invention when measured as described herein exhibits an induction fold of less than or equal to about 3-fold. In some embodiments, a compound of the present invention when measured as described herein exhibits an induction fold of less than or equal to about 2-fold.

In some embodiments, a compound of the present invention is not an inducer of CYP1A2, as measured by CYP1A2 gene expression, for instance, in Example 101. In some embodiments, such may lead to reduced risk for drug-drug interactions. Exemplary such compounds include, e.g., I-296. For instance, in some embodiments, a compound of the present invention when measured as described herein exhibits an induction fold of less than or equal to about 10-fold. In some embodiments, a compound of the present invention when measured as described herein exhibits an induction fold of less than or equal to about 9-fold. In some embodiments, a compound of the present invention when measured as described herein exhibits an induction fold of less than or equal to about 8-fold. In some embodiments, a compound of the present invention when measured as described herein exhibits an induction fold of less than or equal to about 7-fold. In some embodiments, a compound of the present invention when measured as described herein exhibits an induction fold of less than or equal to about 6-fold. In some embodiments, a compound of the present invention when measured as described herein exhibits an induction fold of less than or equal to about 5-fold. In some embodiments, a compound of the present invention when measured as described herein exhibits an induction fold of less than or equal to about 4-fold. In some embodiments, a compound of the present invention when measured as described herein exhibits an induction fold of less than or equal to about 3-fold. In some embodiments, a compound of the present invention when measured as described herein exhibits an induction fold of less than or equal to about 2-fold.

In some embodiments, a compound of the present invention is not an inducer of CYP2C19, as measured by CYP2C19 gene expression, which may lead to reduced risk for drug-drug interactions. For instance, in some embodiments, a compound of the present invention when measured as described herein exhibits an induction fold of less than or equal to about 10-fold. In some embodiments, a compound of the present invention when measured as described herein exhibits an induction fold of less than or equal to about 9-fold. In some embodiments, a compound of the present invention when measured as described herein exhibits an induction fold of less than or equal to about 8-fold. In some embodiments, a compound of the present invention when measured as described herein exhibits an induction fold of less than or equal to about 7-fold. In some embodiments, a compound of the present invention when measured as described herein exhibits an induction fold of less than or equal to about 6-fold. In some embodiments, a compound of the present invention when measured as described herein exhibits an induction fold of less than or equal to about 5-fold. In some embodiments, a compound of the present invention when measured as described herein exhibits an induction fold of less than or equal to about 4-fold. In some embodiments, a compound of the present invention when measured as described herein exhibits an induction fold of less than or equal to about 3-fold. In some embodiments, a compound of the present invention when measured as described herein exhibits an induction fold of less than or equal to about 2-fold.

In some embodiments, a compound of Formula I-1 is administered orally, as described further herein. In some embodiments, a compound of Formula I-1 is administered by a means other than oral administration, as described further herein.

In some embodiments, it has been unexpectedly found that certain compounds of Formula I-1 exhibit improved solubility as compared to c-KIT inhibitors known in the art when measured according to the procedure set forth in Example 102 herein. In some embodiments, compounds of the present invention have a solubility greater than 2.0 μM and less than or equal to 10.0 μM. In some embodiments, compounds of the present invention have a solubility of about 2.5 μM, about 3.0 μM, about 3.5 μM, about 4.0 μM, about 4.5 μM, about 5.0 μM, about 5.5 μM, about 6.0 μM, about 6.5 μM, about 7.0 μM, about 7.5 μM, about 8.0 μM, about 9.0 μM, about 9.5 μM, or about 10.0 μM. In some embodiments, compounds of the present invention have a solubility greater than 10 μM and less than or equal to 50 μM. In some embodiments, compounds of the present invention have a solubility of about 15 μM, about 20 μM, about 25 μM, about 30 μM, about 35 μM, about 40 μM, about 45 μM, or about 50 μM. In some embodiments, compounds of the present invention have a solubility greater than 50 μM. In some embodiments, compounds of the present invention have a solubility of about 60 μM, about 70 μM, about 80 μM, about 90 μM, about 100 μM, about 200 μM, about 300 μM, about 400 μM, 500 μM, about 600 μM, about 700 μM, about 800 μM, about 900 μM, about 1000 μM, about 1500 μM or about 2000 μM.

As described above and herein, in some embodiments, R¹ represents independently for each occurrence halogen, —CN, —OR, C₁₋₆ alkyl, or C₁₋₆ haloalkyl. In some embodiments, R¹ represents independently for each occurrence halogen. In some embodiments, R¹ represents independently for each occurrence fluoro, chloro, or bromo. In some embodiments, R¹ represents independently for each occurrence —CN. In some embodiments, R¹ represents independently for each occurrence —OR. In some embodiments, R¹ represents independently for each occurrence C₁₋₆ alkyl or C₁₋₆ haloalkyl. In some embodiments, R¹ represents independently for each occurrence C₁₋₆ alkyl. In some such embodiments, R¹ represents independently for each occurrence methyl. In some embodiments, R¹ represents independently for each occurrence C₁₋₆ haloalkyl. In some such embodiments, R¹ represents independently for each occurrence —CF₃, —CF₂H, or —CFH₂. In some embodiments, at least one R¹ is fluoro. In some embodiments, at least one R¹ is chloro. In some embodiments, at least one R¹ is bromo. In some embodiments, at least one R¹ is —CN. In some embodiments, at least one R¹ is —OR. In some such embodiments, at least one R¹ is —OMe. In some embodiments, at least one R¹ is methyl. In some embodiments, at least one R¹ is —CF₃. In some embodiments, at least one R¹ is —CF₂H. In some embodiments, at least one R¹ is —CFH₂.

In some embodiments, R¹ is as depicted in Table 1, below.

In some embodiments, R¹ is as described above and herein, wherein m is 0, 1, 2, 3, or 4. In some such embodiments, m is 0. In some such embodiments, m is 1. In some such embodiments, m is 2. In some such embodiments, m is 3. In some such embodiments, m is 4.

As described above and herein, R² is C₁₋₆ aliphatic, phenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; or L¹-R⁴, wherein R² is substituted with p occurrences of R⁶.

In some embodiments, R² is C₁₋₆ aliphatic. In some such embodiments, R² is C₁₋₆ alkyl. In some such embodiments, R² is methyl, ethyl, or propyl. In some embodiments, R² is —CH₂CH(OH)CH₃.

In some embodiments, R² is phenyl.

In some embodiments, R² is a 3-7 membered saturated or partially unsaturated monocyclic carbocyclic ring. In some embodiments, R² is a 3-membered saturated monocyclic carbocyclic ring. In some embodiments, R² is a 4-membered saturated monocyclic carbocyclic ring. In some embodiments, R² is a 5-membered saturated monocyclic carbocyclic ring. In some embodiments, R² is a 6-membered saturated monocyclic carbocyclic ring. In some embodiments, R² is a 7-membered saturated monocyclic carbocyclic ring.

In some embodiments, R² is a 3-4 membered saturated monocyclic carbocyclic ring substituted with 0-2 occurrences of R⁶. In some embodiments, R² is a 3 membered saturated monocyclic carbocyclic ring substituted with 0-2 occurrences of R⁶. In some embodiments, R² is a 4 membered saturated monocyclic carbocyclic ring substituted with 0-2 occurrences of R⁶. In some embodiments, R² is a 3-4 membered saturated monocyclic carbocyclic ring substituted with 1-2 occurrences of R⁶. In some such embodiments, at least one R⁶ is fluoro. In some such embodiments, two R⁶ are fluoro. In some such embodiments, at least one R⁶ is methyl. In some such embodiments, at least one R⁶ is —CN.

In some embodiments, R² is a 5 membered saturated monocyclic carbocyclic ring substituted with 0-2 occurrences of R⁶. In some embodiments, R² is a 5 membered saturated monocyclic carbocyclic ring substituted with 1-2 occurrences of R⁶. In some such embodiments, at least one R⁶ is fluoro. In some such embodiments, two R⁶ are fluoro. In some such embodiments, at least one R⁶ is methyl. In some such embodiments, at least one R⁶ is —CN.

In some embodiments, R² is a 3-7 membered saturated or partially unsaturated monocyclic heterocyclic ring having 1-2 heteroatoms independently selected from nitrogen, oxygen, and sulfur. In some embodiments, R² is a 3 membered saturated monocyclic heterocyclic ring having 1-2 heteroatoms independently selected from nitrogen, oxygen, and sulfur. In some embodiments, R² is a 4 membered saturated monocyclic heterocyclic ring having 1-2 heteroatoms independently selected from nitrogen, oxygen, and sulfur. In some embodiments, R² is a 4 membered saturated monocyclic heterocyclic ring having 1 heteroatom independently selected from nitrogen, oxygen, and sulfur. In some embodiments, R² is a 4 membered saturated monocyclic heterocyclic ring having 1 heteroatom independently selected from nitrogen. In some embodiments, R² is a 5 membered saturated or partially unsaturated monocyclic heterocyclic ring having 1-2 heteroatoms independently selected from nitrogen, oxygen, and sulfur. In some embodiments, R² is a 6 membered saturated or partially unsaturated monocyclic heterocyclic ring having 1-2 heteroatoms independently selected from nitrogen, oxygen, and sulfur. In some embodiments, R² is a 7 membered saturated or partially unsaturated monocyclic heterocyclic ring having 1-2 heteroatoms independently selected from nitrogen, oxygen, and sulfur.

In some embodiments, R² is a 4-5 membered saturated monocyclic heterocyclic ring having 1-2 heteroatoms independently selected from nitrogen, oxygen, and sulfur substituted with 0-2 occurrences of R⁶. In some embodiments, R² is a 4-5 membered saturated monocyclic heterocyclic ring having 1-2 heteroatoms independently selected from nitrogen, oxygen, and sulfur substituted with 1-2 occurrences of R⁶. In some such embodiments, at least one R⁶ is fluoro. In some such embodiments, two R⁶ are fluoro. In some such embodiments, at least one R⁶ is methyl. In some such embodiments, at least one R⁶ is —CN.

In some embodiments, R² is a 5-6 membered monocyclic heteroaryl ring having 1-4 heteroatoms independently selected from nitrogen, oxygen, and sulfur. In some embodiments, R² is a 5 membered monocyclic heteroaryl ring having 1-4 heteroatoms independently selected from nitrogen, oxygen, and sulfur. In some embodiments, R² is a 6 membered monocyclic heteroaryl ring having 1-4 heteroatoms independently selected from nitrogen, oxygen, and sulfur.

In some embodiments, R² is an 8-10 membered bicyclic heteroaryl ring having 1-5 heteroatoms independently selected from nitrogen, oxygen, and sulfur. In some embodiments, R² is an 8-10 membered bicyclic heteroaryl ring having 1-3 heteroatoms independently selected from nitrogen, oxygen, and sulfur. In some embodiments, R² is an 8 membered bicyclic heteroaryl ring having 1-5 heteroatoms independently selected from nitrogen, oxygen, and sulfur. In some embodiments, R² is a 9 membered bicyclic heteroaryl ring having 1-5 heteroatoms independently selected from nitrogen, oxygen, and sulfur. In some embodiments, R² is a 10 membered bicyclic heteroaryl ring having 1-5 heteroatoms independently selected from nitrogen, oxygen, and sulfur.

In some embodiments, R² is a 5-10 membered saturated or partially unsaturated bridged bicyclic ring having 0-3 heteroatoms independently selected from nitrogen, oxygen, and sulfur. In some embodiments, R² is a 5-10 membered saturated or partially unsaturated bridged bicyclic ring having 1-3 heteroatoms independently selected from nitrogen, oxygen, and sulfur. In some embodiments, R² is a 5-10 membered saturated bridged bicyclic ring having 0-3 heteroatoms independently selected from nitrogen, oxygen, and sulfur. In some embodiments, R² is a 5-10 membered partially unsaturated bridged bicyclic ring having 0-3 heteroatoms independently selected from nitrogen, oxygen, and sulfur. In some embodiments, R² is a 5-10 membered saturated or partially unsaturated bridged bicyclic ring having 0 heteroatoms. In some embodiments, R² is a 5-10 membered saturated or partially unsaturated bridged bicyclic ring having one heteroatom independently selected from nitrogen, oxygen, and sulfur. In some embodiments, R² is a 5-10 membered saturated or partially unsaturated bridged bicyclic ring having two heteroatoms independently selected from nitrogen, oxygen, and sulfur. In some embodiments, R² is a 5-10 membered saturated or partially unsaturated bridged bicyclic ring having three heteroatoms independently selected from nitrogen, oxygen, and sulfur.

In some embodiments, R² is a 6-11 membered saturated or partially unsaturated spirocyclic ring having 0-3 heteroatoms independently selected from nitrogen, oxygen, and sulfur. In some embodiments, R² is a 6-11 membered saturated or partially unsaturated spirocyclic ring having 0 heteroatoms. In some embodiments, R² is a 6-11 membered saturated or partially unsaturated spirocyclic ring having 1-3 heteroatoms independently selected from nitrogen, oxygen, and sulfur.

In some embodiments, R² is a 6-11 membered saturated or partially unsaturated bicyclic heterocyclic ring having 1-3 heteroatoms independently selected from nitrogen, oxygen, and sulfur.

In some embodiments, R² is L¹-R⁴.

As described above and defined herein, L¹ represents is a C₁₋₂ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₂ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—.

As described above and defined herein, R⁴ is a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur. In some embodiments, R⁴ is a 3-7 membered saturated or partially unsaturated monocyclic carbocyclic ring. In some embodiments, R⁴ is a 3-7 membered saturated or partially unsaturated monocyclic heterocyclic ring having 1-2 heteroatoms independently selected from nitrogen, oxygen, and sulfur.

As described above and herein, R⁶ represents independently for each occurrence oxo, halogen, C₁₋₆ aliphatic, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or optionally substituted phenyl;

In some embodiments, at least one R⁶ is halogen. In some embodiments, at least one R⁶ is fluoro. In some embodiments, at least two R⁶ are fluoro. In some embodiments, at least one R⁶ is methyl. In some embodiments, at least one R⁶ is cyano.

In some embodiments, R² is selected from:

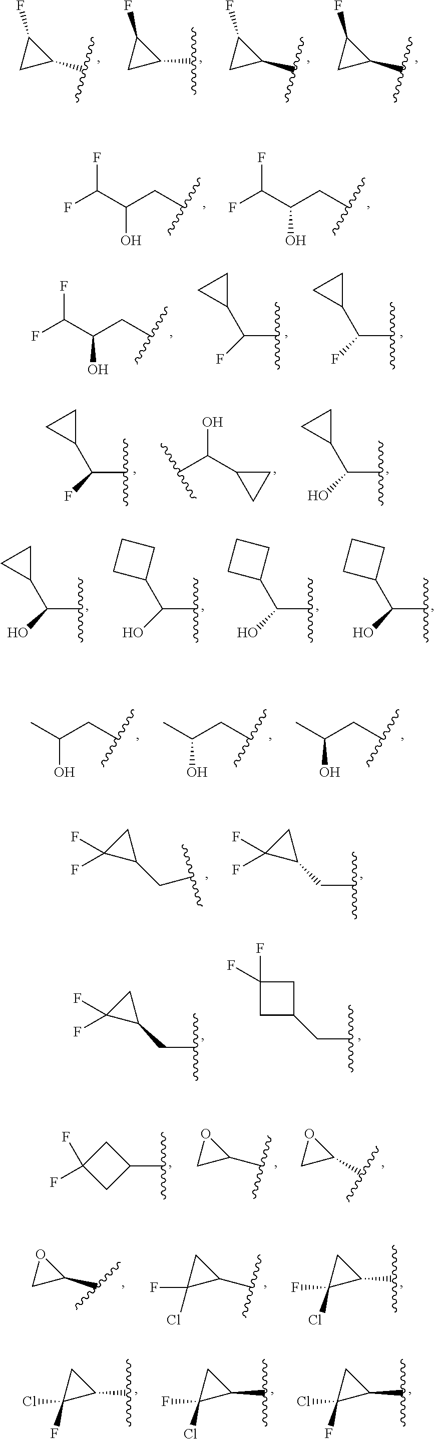

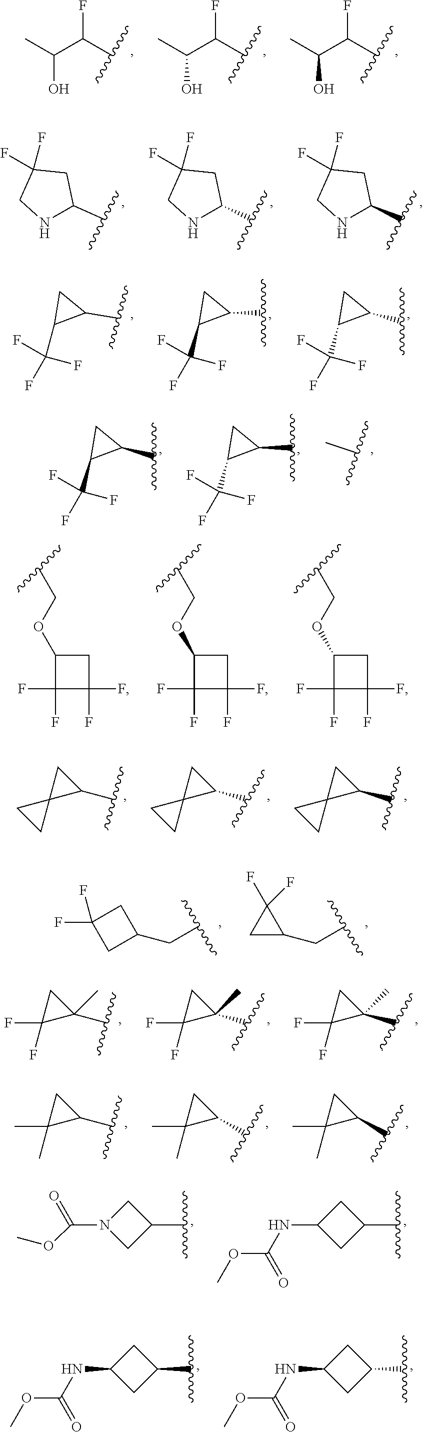

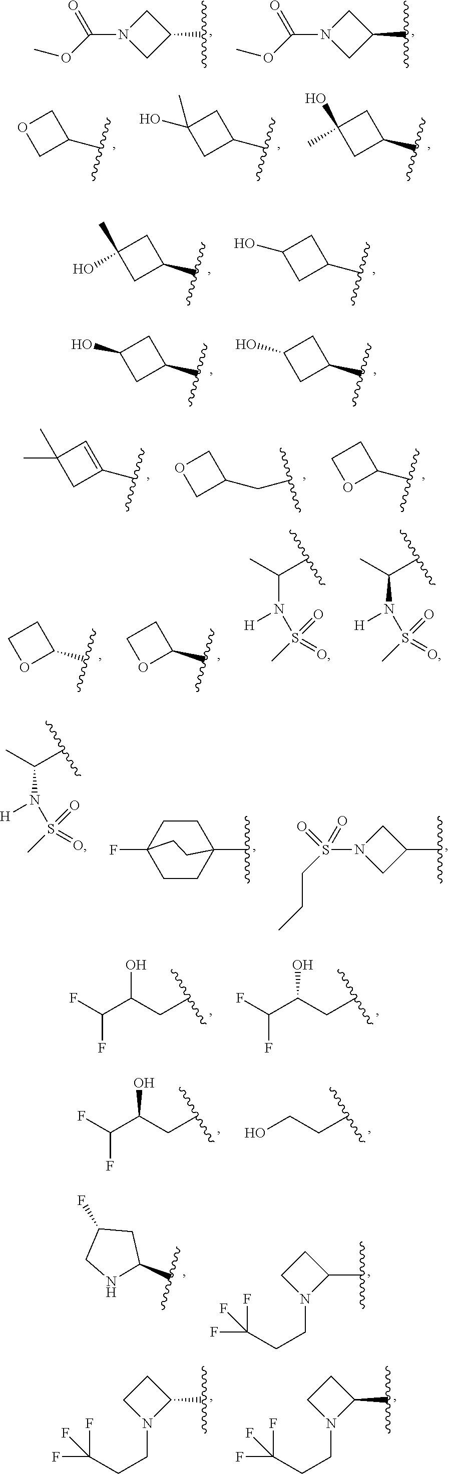

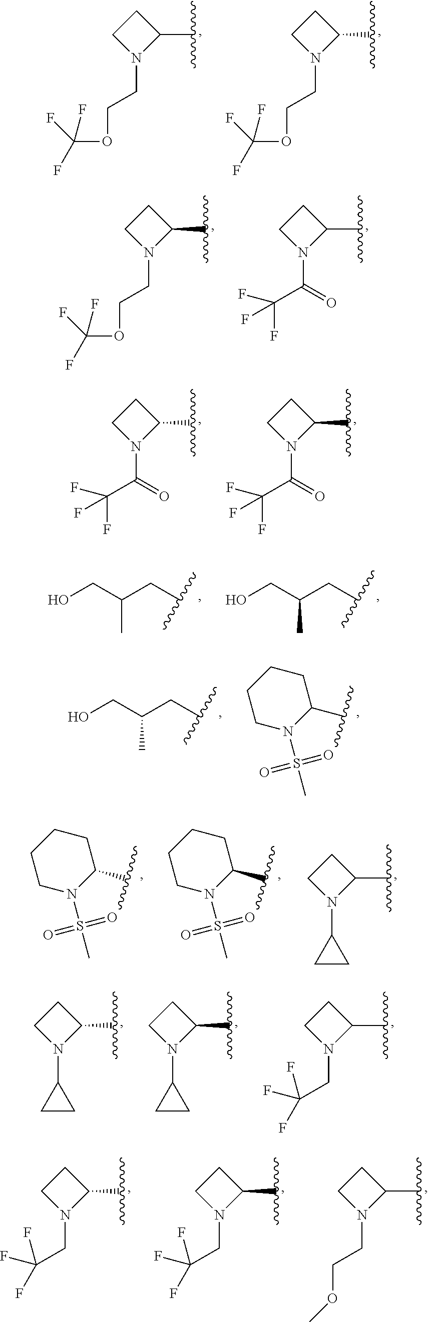

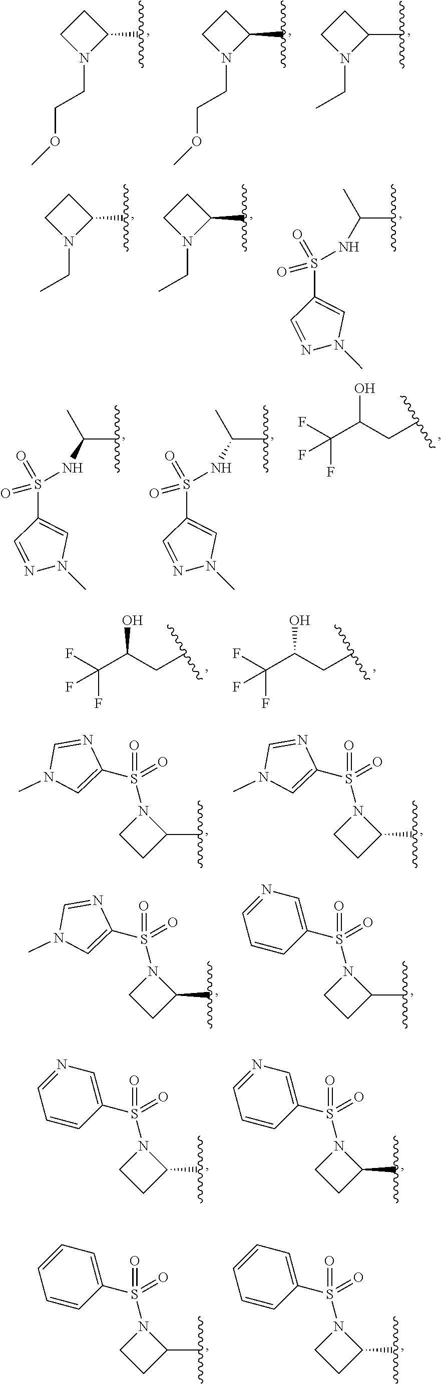

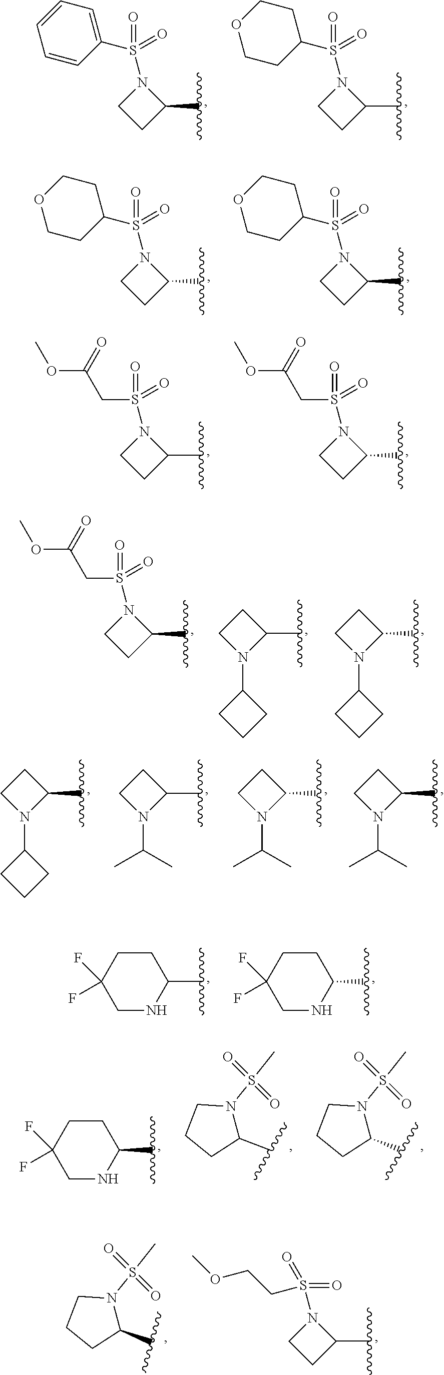

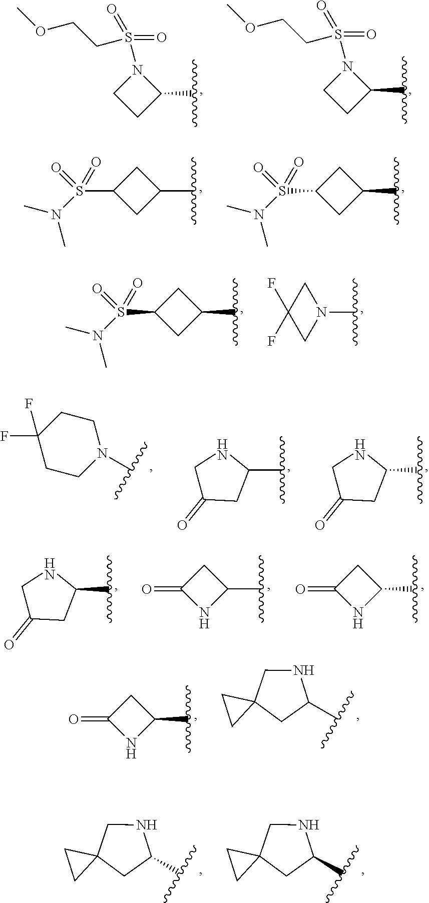

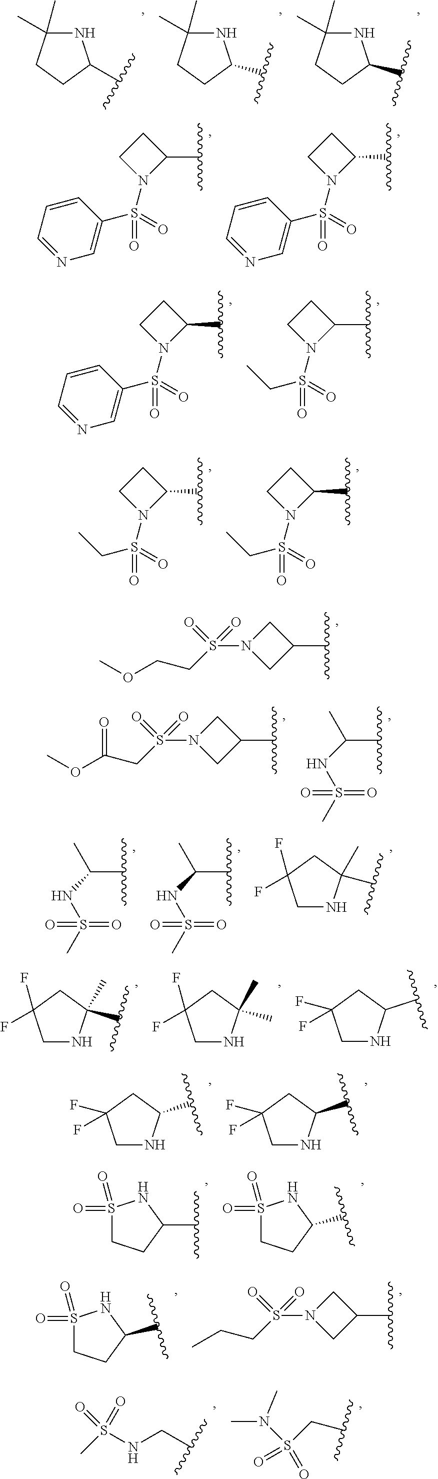

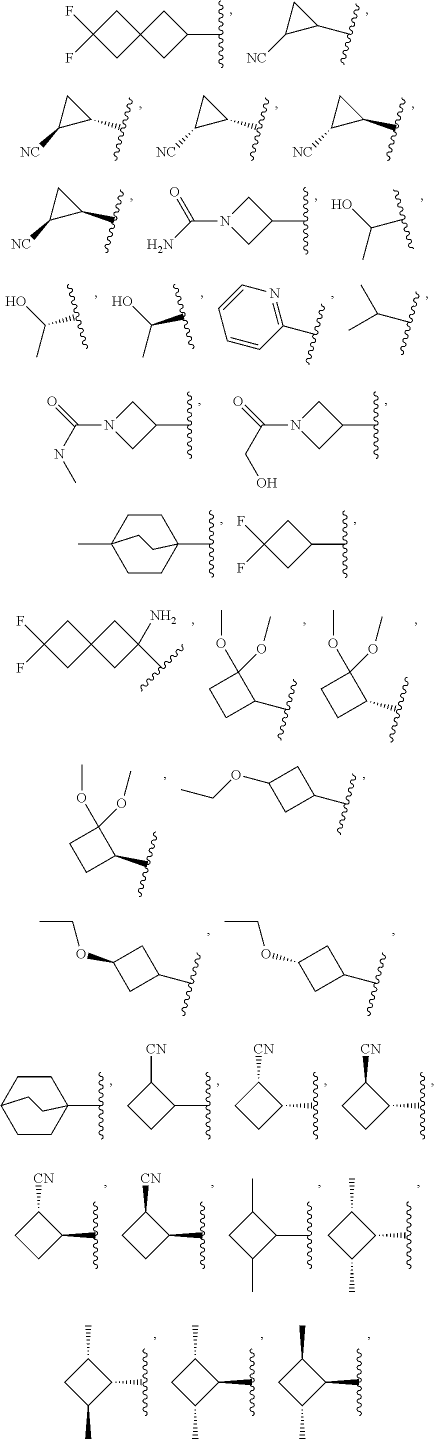

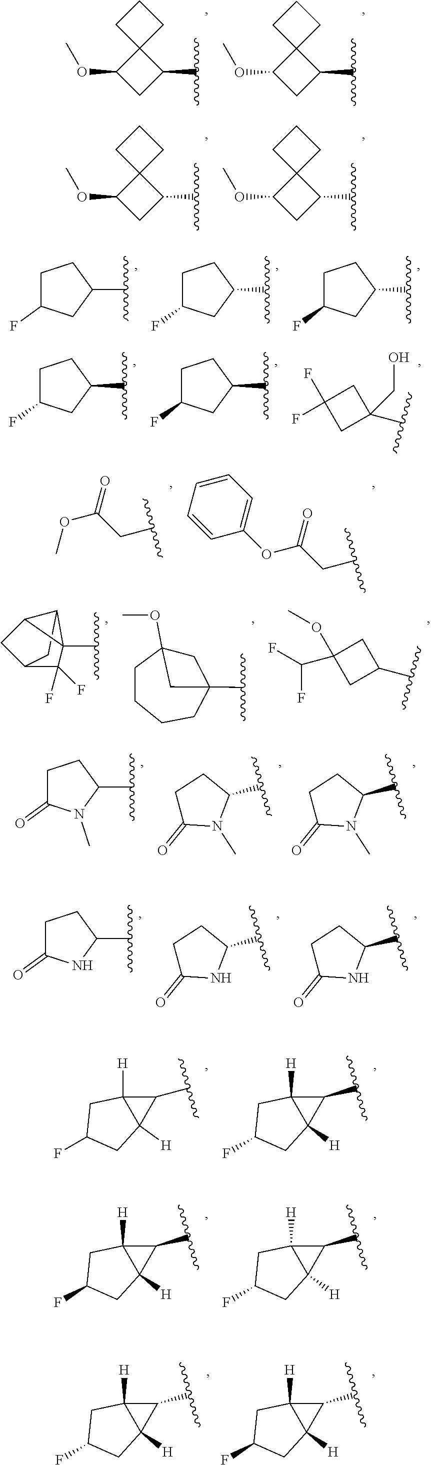

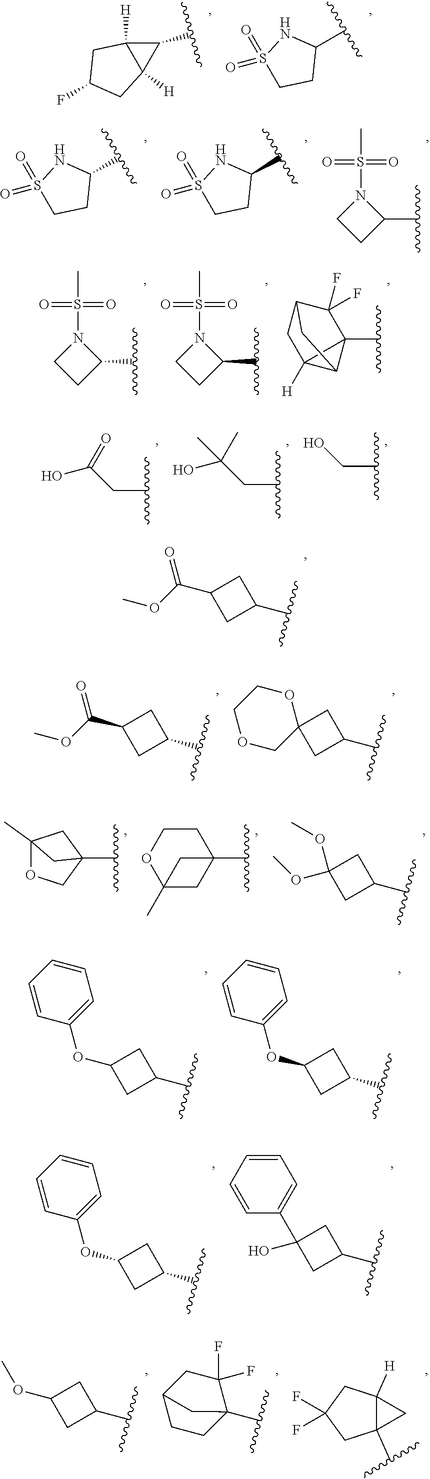

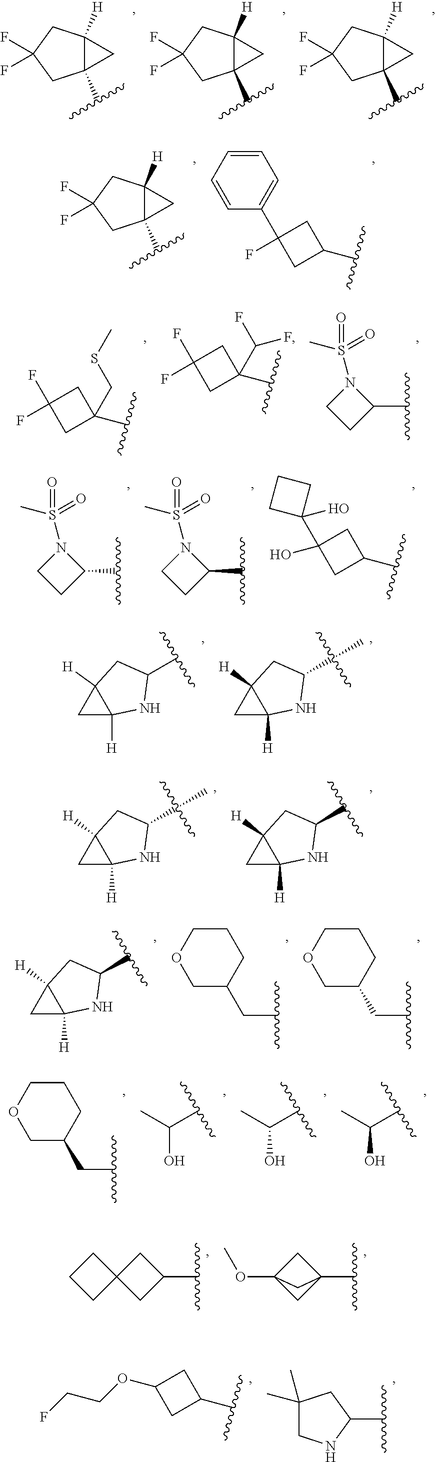

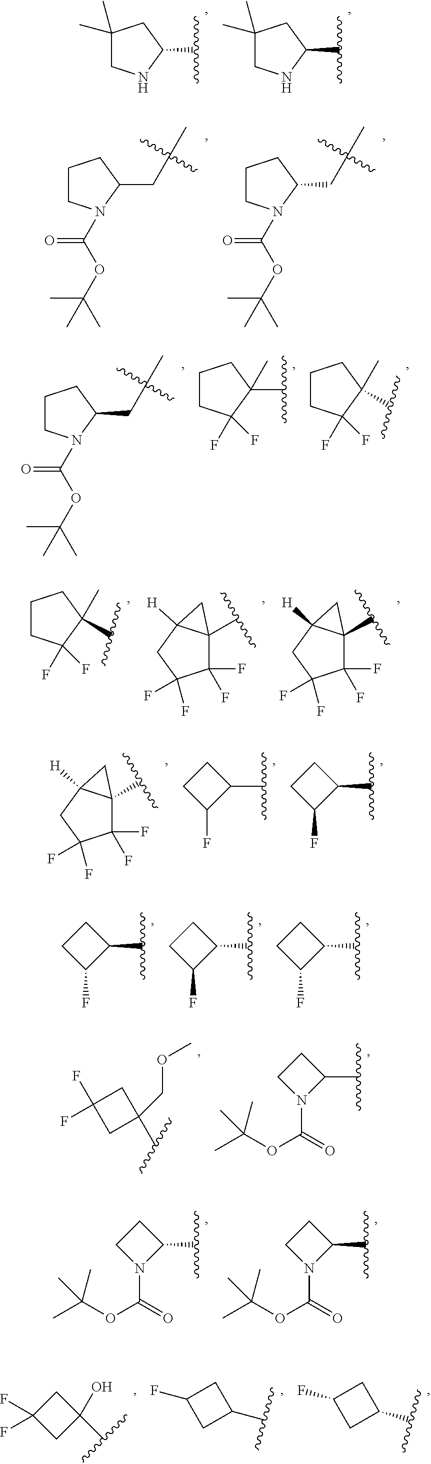

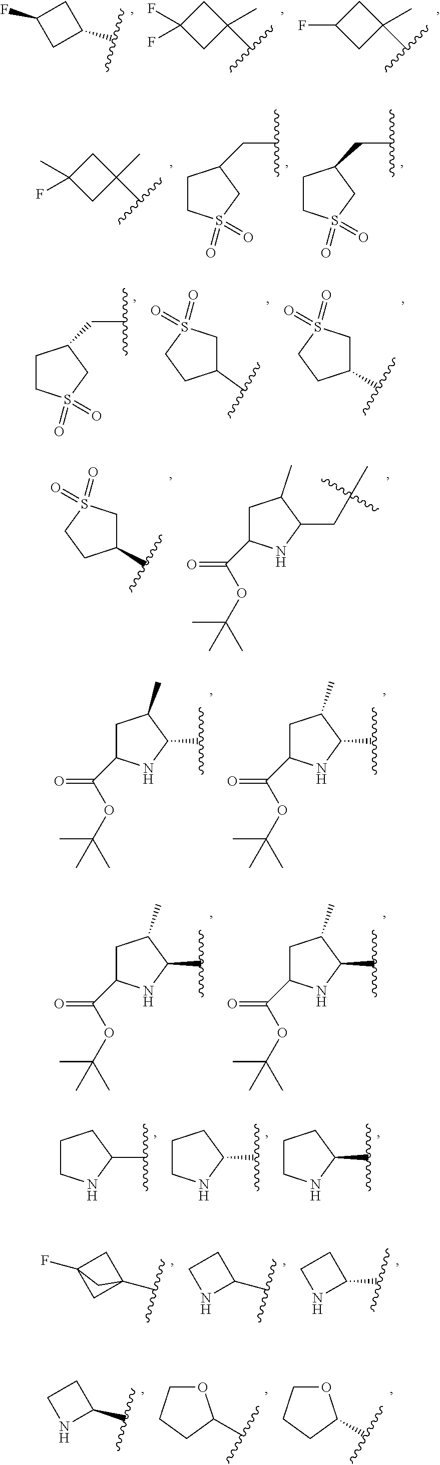

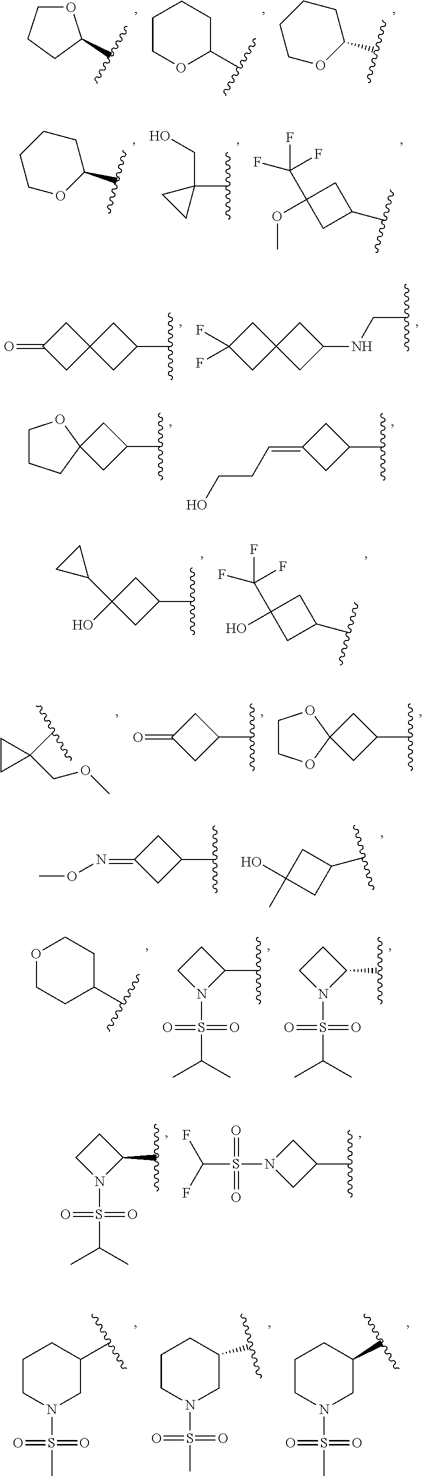

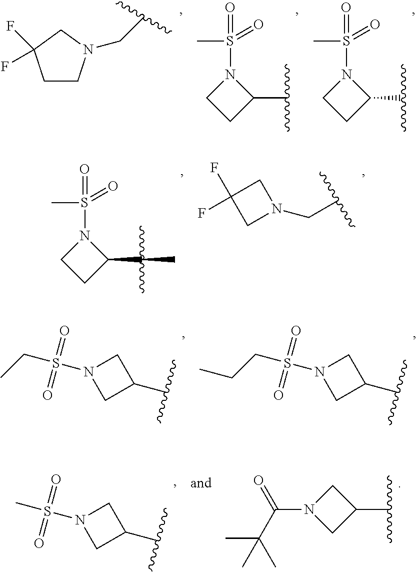

In some embodiments, R² is selected from:

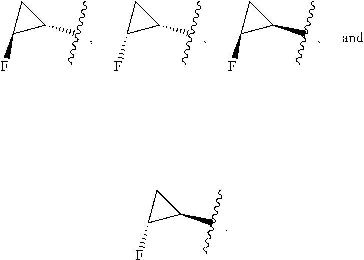 [SMILES: *[C@H]1C[C@@H]1F ; *[C@H]1C[C@H]1F ; *[C@@H]1C[C@@H]1F ; *[C@@H]1C[C@H]1F]

In some embodiments, R² is selected from:

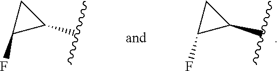 [SMILES: *[C@H]1C[C@@H]1F ; *[C@@H]1C[C@H]1F]

In some embodiments, R² is:

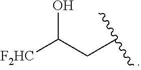 [SMILES: *CC(O)C(F)F]

In some embodiments, R² is selected from:

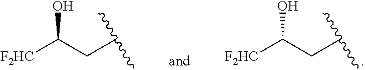 [SMILES: *C[C@H](O)C(F)F ; *C[C@@H](O)C(F)F]

In some embodiments, R² is as described above and herein, wherein R² is substituted with p occurrences of R⁶. In some such embodiments, p is 0. In some such embodiments, p is 1. In some such embodiments, p is 2. In some such embodiments, p is 3. In some such embodiments, p is 4. In some such embodiments, p is 5.

In some embodiments, R² is as depicted in Table 1, below.

As described above and herein, R^{A} is of any of the following structures:

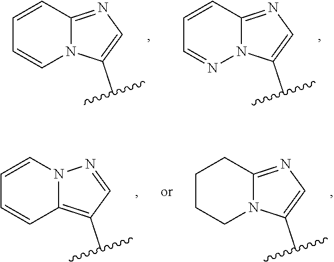 [SMILES: *c1cnc2ccccn12 ; *c1cnc2cccnn12 ; *c1cnn2ccccc12 ; *c1cnc2n1CCCC2]

each of which is substituted by n occurrences of R³.

In some embodiments, R^{A} is

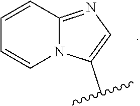 [SMILES: *c1cnc2ccccn12]

In some embodiments, R^{A} s

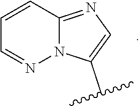 [SMILES: *c1cnc2cccnn12]

In some embodiments, R^{A} is

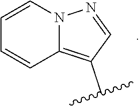 [SMILES: *c1cnn2ccccc12]

In some embodiments, R^{A} is

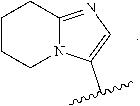 [SMILES: *c1cnc2n1CCCC2]

In some embodiments, R^{A} is any of those depicted in Table 1 below.

As described above and herein, each R^{A} is substituted by n occurrences of R³. In some embodiments, n is 0. In some embodiments, n is 1. In some embodiments, n is 2. In some embodiments, n is 3. In some embodiments, n is 4. In some embodiments, n is 5.

As described above and herein, R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, -L²-R⁵, or an optionally substituted group selected from C₁₋₆ aliphatic, C₁₋₆ haloaliphatic, phenyl, naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; each of which is substituted with r instances of R; or: two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur, each of which is substituted with r instance of R.

In some embodiments, R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or -L²-R⁵.

In some embodiments, R³ represents independently for each occurrence halogen. In some embodiments, at least one R³ is fluoro. In some embodiments, at least one R³ is chloro. In some embodiments, at least one R³ is bromo. In some embodiments, at least one R³ is cyano. In some embodiments, at least one R³ is —OR. In some embodiments, wherein at least one R³ is —OR, wherein R is C₁₋₆ alkyl. In some embodiments, at least one R³ is —OR, wherein R is methyl, ethyl, or propyl. In some embodiments, at least one R³ is —OR, wherein R is methyl. In some embodiments, at least one R³ is —OR, wherein R is ethyl. In some embodiments, at least one R³ is —OR, wherein R is propyl. In some embodiments, at least one R³ is —OCR₃, wherein at least one R is fluoro. In some embodiments, at least one R³ is —NR₂. In some embodiments, wherein at least one R³ is —NR₂, at least one R is hydrogen. In some embodiments, wherein at least one R³ is —NR₂, at least one R is methyl or ethyl. In some embodiments, wherein at least one R³ is —NR₂, at least one R is optionally substituted phenyl. In some embodiments, wherein at least one R³ is —NR₂, the two R groups on the same nitrogen are taken together with the nitrogen to form an optionally substituted 4-7 membered monocyclic saturated ring having 1-3 heteroatoms independently selected from nitrogen, oxygen, and sulfur. In some embodiments, at least one R³ is —N(R)S(O)₂R. In some such embodiments, each R is independently hydrogen, C₁₋₆ alkyl, C₃₋₆ cycloalkyl, naphthalenyl, or a 5-membered heteroaryl ring having one, two, or three heteroatoms independently selected from nitrogen, oxygen, or sulfur.

In some embodiments, at least one R³ is -L²-R⁵. In some such embodiments, one, two, or three methylene units of L² are independently replaced by —O— or -Cy-. In some such embodiments, one, two, or three methylene units of L² are independently replaced by —N(R)— or -Cy-.

In some embodiments, at least one R³ is a 3-7 membered saturated or partially unsaturated monocyclic heterocyclic ring having 1-2 heteroatoms independently selected from nitrogen, oxygen, and sulfur. In some such embodiments, at least one R³ is oxetane.

In some embodiments, at least one R³ is —CF₃, —CF₂H, or —CFH₂.

In some embodiments, R³ represents independently for each occurrence C₁₋₆ aliphatic or C₁₋₆ haloaliphatic.

In some embodiments, R³ represents independently for each occurrence phenyl or naphthalenyl.

In some embodiments, R³ represents independently for each occurrence a 3-7 membered saturated or partially unsaturated monocyclic carbocyclic ring.

In some embodiments, R³ represents independently for each occurrence a 3-7 membered saturated or partially unsaturated monocyclic heterocyclic ring having 1-2 heteroatoms independently selected from nitrogen, oxygen, and sulfur.

In some embodiments, R³ represents independently for each occurrence a 5-6 membered monocyclic heteroaryl ring having 1-4 heteroatoms independently selected from nitrogen, oxygen, and sulfur.

In some embodiments, R³ represents independently for each occurrence an 8-10 membered bicyclic heteroaryl ring having 1-5 heteroatoms independently selected from nitrogen, oxygen, and sulfur.

In some embodiments, R³ represents independently for each occurrence a 5-8 membered saturated or partially unsaturated bridged bicyclic ring having 0-3 heteroatoms independently selected from nitrogen, oxygen, and sulfur.

In some embodiments, R³ represents independently for each occurrence a 6-11 membered saturated or partially unsaturated spirocyclic ring having 0-3 heteroatoms independently selected from nitrogen, oxygen, and sulfur.

In some embodiments, R³ represents independently for each occurrence or a 6-11 membered saturated or partially unsaturated bicyclic heterocyclic ring having 1-3 heteroatoms independently selected from nitrogen, oxygen, and sulfur.

In some embodiments, two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur. In some embodiments, two R³ groups on adjacent carbon atoms are taken together with the carbon atoms to which they attach to form an optionally substituted 4-7 membered saturated monocyclic ring having 1-3 heteroatoms independently selected from nitrogen, oxygen, and sulfur. In some embodiments, two R³ groups on adjacent carbon atoms are taken together with the carbon atoms to which they attach to form an optionally substituted 5 membered saturated monocyclic ring having 1-3 heteroatoms independently selected from nitrogen, oxygen, and sulfur. In some embodiments, two R³ groups on adjacent carbon atoms are taken together with the carbon atoms to which they attach to form an optionally substituted 4-7 membered saturated monocyclic ring having one heteroatom independently selected from oxygen.

In some embodiments, R³ represents independently for each occurrence -L²-R⁵.

As described above and herein, each L² represents independently for each occurrence a C₁₋₆ bivalent saturated or unsaturated, straight or branched hydrocarbon chain wherein one, two, or three methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂— or -Cy-. In some embodiments, each L² represents independently for each occurrence a C₁₋₆ bivalent saturated or straight or branched hydrocarbon chain wherein one, two, or three methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂— or -Cy-.

As described above and herein, -Cy- represents independently for each occurrence phenyl, a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur.

In some embodiments, -Cy- represents independently for each occurrence phenyl.

In some embodiments, -Cy- represents independently for each occurrence a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, -Cy- represents independently for each occurrence a 3-7 membered saturated monocyclic carbocyclic ring. In some embodiments, -Cy- represents independently for each occurrence a 3-7 membered saturated or partially unsaturated monocyclic heterocyclic ring having 1-2 heteroatoms independently selected from nitrogen, oxygen, and sulfur.

As described above and herein, R⁵ represents independently for each occurrence hydrogen, OR, C₁₋₆ aliphatic, C₁₋₆ haloaliphatic, or phenyl fused to a 5-6 membered saturated heterocyclic ring having 1-2 heteroatoms independently selected from nitrogen, oxygen, and sulfur. In some embodiments, R⁵ represents independently for each occurrence hydrogen. In some embodiments, R⁵ represents independently for each occurrence OR. In some such embodiments, R⁵ represents independently for each occurrence OH or OMe. In some embodiments, R⁵ represents independently for each occurrence C₁₋₆ aliphatic or C₁₋₆ haloaliphatic. In some embodiments, R⁵ represents independently for each occurrence phenyl fused to a 5-6 membered saturated heterocyclic ring having 1-2 heteroatoms independently selected from nitrogen, oxygen, and sulfur.

In some embodiments, each R³ group is independently substituted with r instances of R. In some embodiments, r is 0. In some embodiments, r is 1. In some embodiments, r is 2. In some embodiments, r is 3. In some embodiments, r is 4. In some embodiments, r is 5.

In some embodiments, R³ is independently for each occurrence selected from:

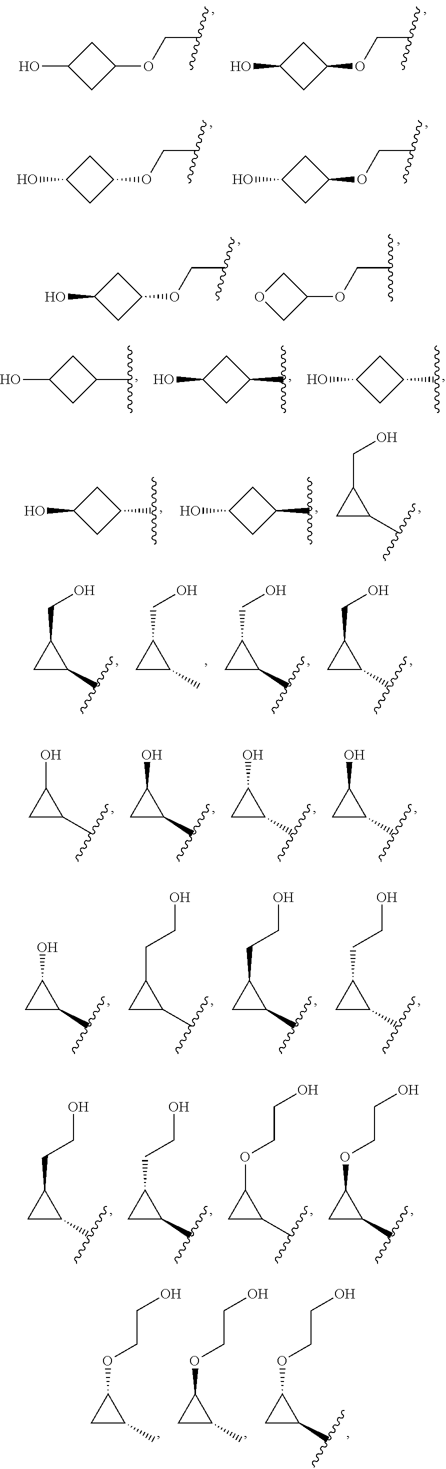

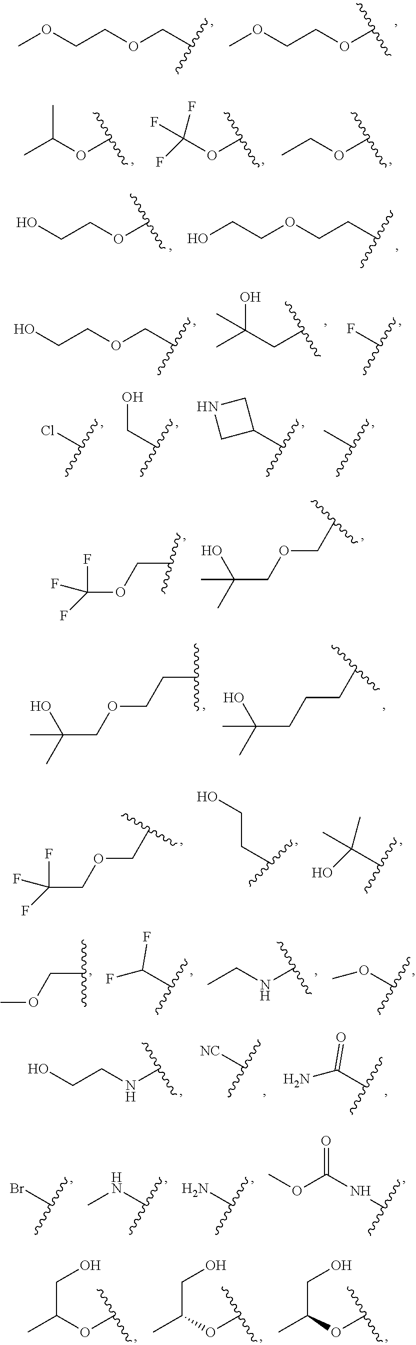

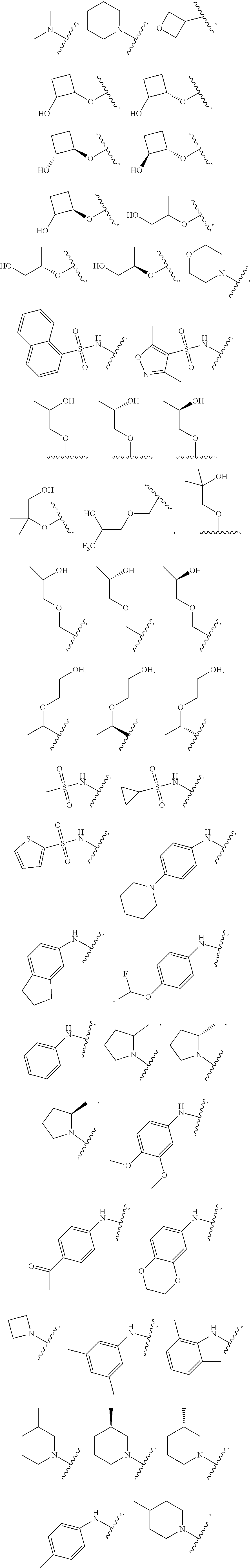

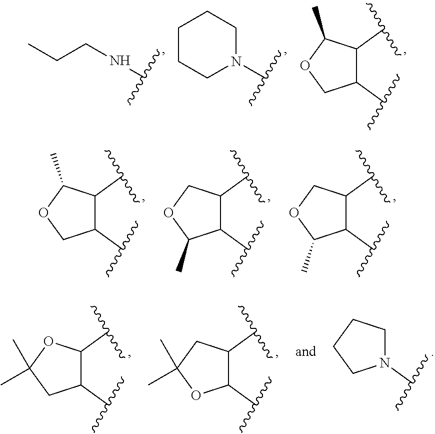

In some embodiments, R³ is —OMe or -OiPr.

In some embodiments, R³ is as depicted in Table 1 below.

As defined above and herein, R is independently for each occurrence hydrogen, —CN, halogen, or an optionally substituted group selected from C₁₋₆ aliphatic; C₁₋₆ haloaliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or: two R groups on the same nitrogen are independently taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur.

In some embodiments, R is independently for each occurrence hydrogen, —CN, halogen, or an optionally substituted group selected from C₁₋₆ aliphatic or C₁₋₆ haloaliphatic. In some embodiments, R is independently for each occurrence hydrogen. In some embodiments, R is independently for each occurrence halogen, for instance fluoro. In some embodiments, R is independently for each occurrence an optionally substituted group selected from C₁₋₆ alkyl. In some such embodiments, R is independently for each occurrence methyl.

In some embodiments, R is independently for each occurrence an optionally substituted group selected from phenyl or naphthalenyl.

In some embodiments, R is independently for each occurrence an optionally substituted group selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic carbocyclic ring having 1-2 heteroatoms independently selected from nitrogen, oxygen, and sulfur.

In some embodiments, two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur.

In some embodiments, a compound of the present disclosure is represented by any of the following or a pharmaceutically acceptable salt thereof:

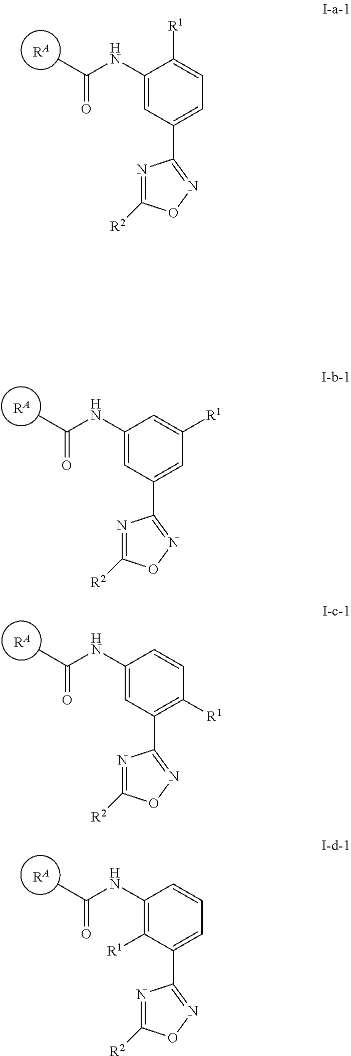

wherein R^{A}, R¹, and R² are as defined above and described herein.

In some embodiments, a compound of the present disclosure is represented by either of the following or a pharmaceutically acceptable salt thereof:

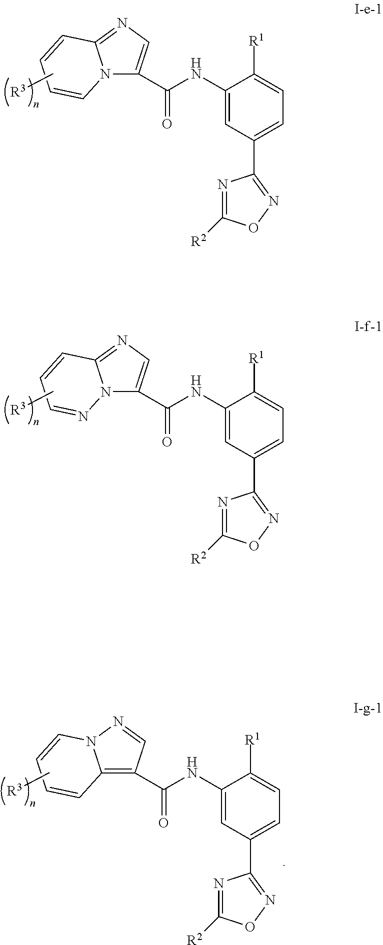

wherein R¹, R², R³, and n are as defined above and described herein.

In some embodiments, a compound of the present disclosure is represented by either of the following or a pharmaceutically acceptable salt thereof:

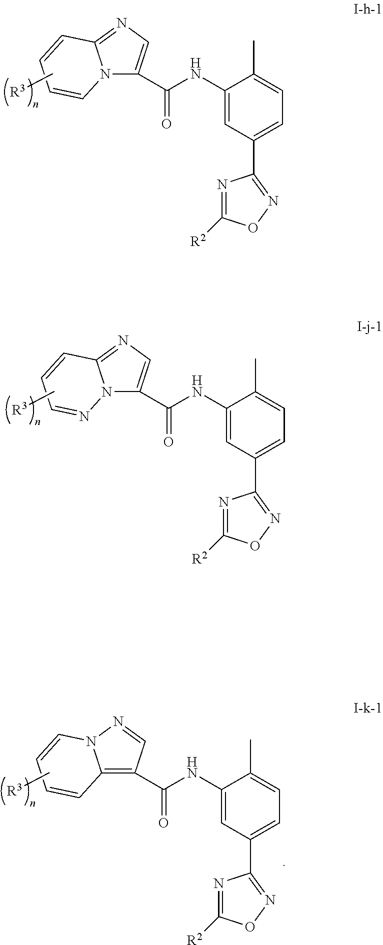

wherein R², R³, and n are as defined above and described herein.

In some embodiments, a compound of the present disclosure is represented by either of the following or a pharmaceutically acceptable salt thereof:

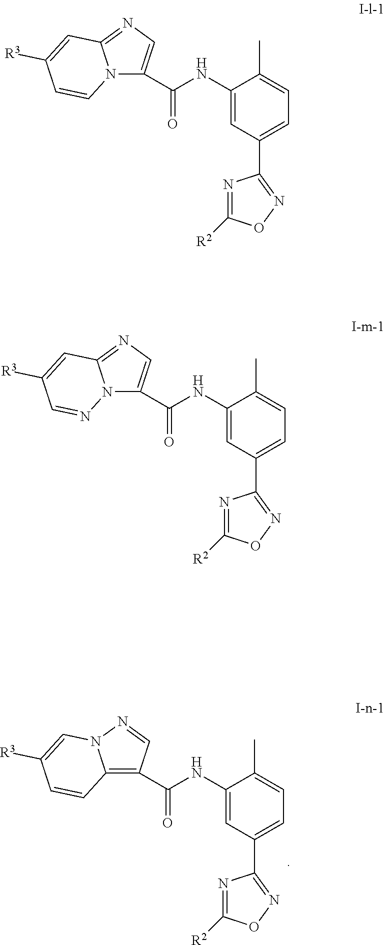

wherein R² and R³ are as defined above and described herein. In some such embodiments, R³ is -OiPr, —CH₂O(CH₂)₂OH, or —CH₂OCH₂C(CH₃)₂OH, and R² is selected from

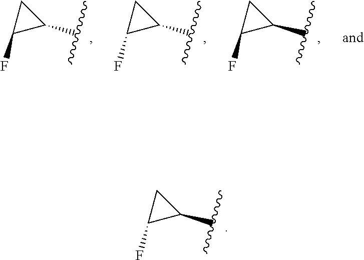 [SMILES: *[C@H]1C[C@@H]1F ; *[C@H]1C[C@H]1F ; *[C@@H]1C[C@@H]1F ; *[C@@H]1C[C@H]1F]

In some embodiments, a compound of the present disclosure is represented by either of the following or a pharmaceutically acceptable salt thereof:

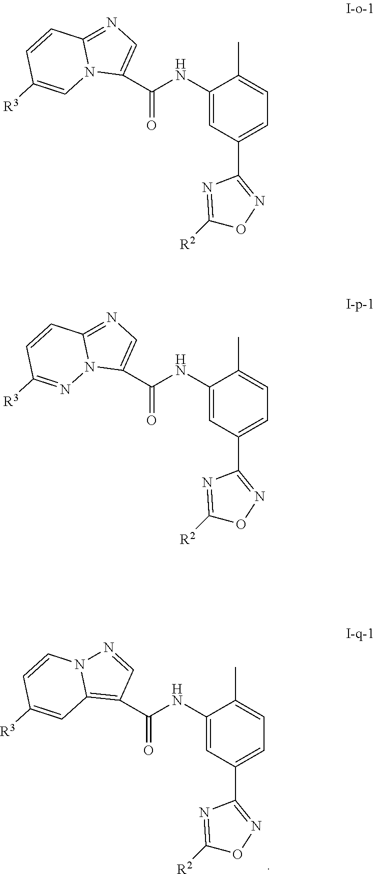

wherein R² and R³ are as defined above and described herein.

In some embodiments, a compound of the present disclosure is represented by either of the following or a pharmaceutically acceptable salt thereof:

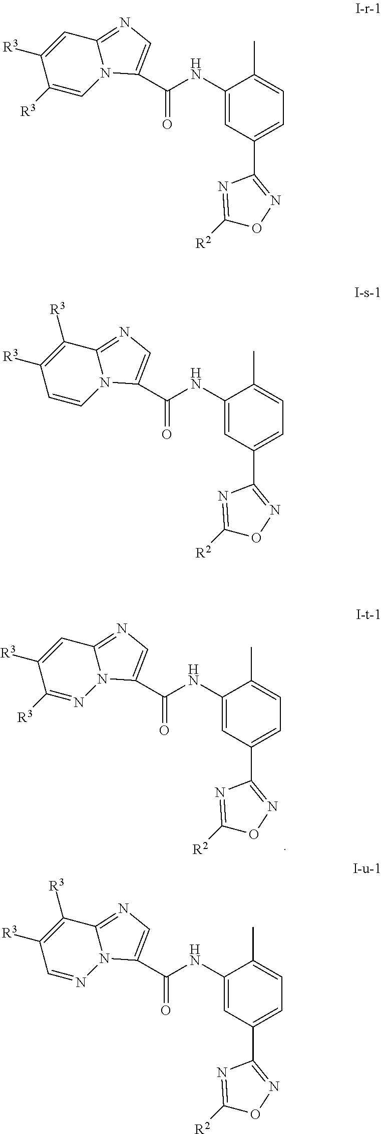

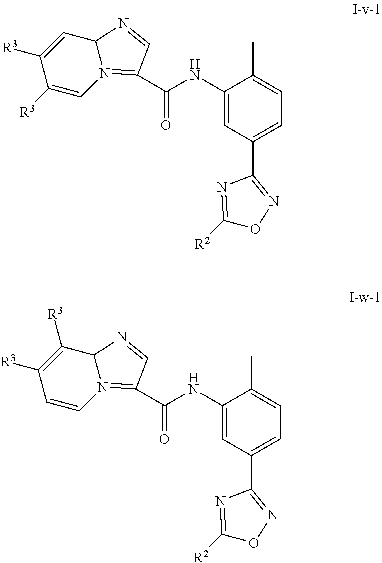

wherein R² and R³ are as defined above and described herein.

In some embodiments, a compound of the present disclosure is represented by either of the following or a pharmaceutically acceptable salt thereof:

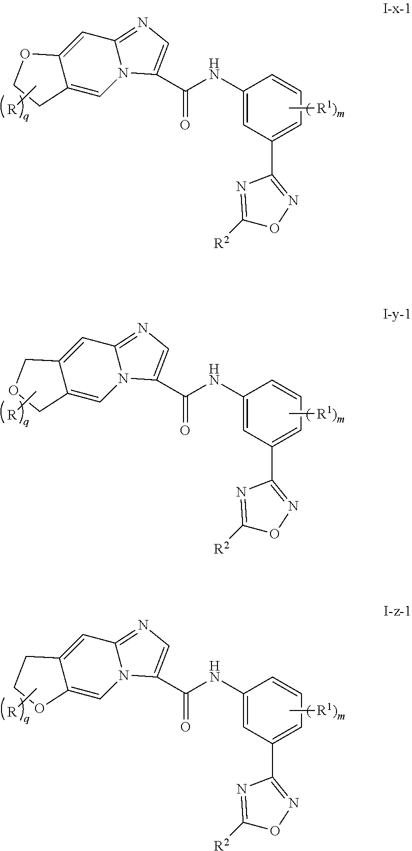

wherein R¹, R², R, and m are as defined above and described herein and q is 0, 1, 2, 3 or 4.

In some embodiments, a compound of the present disclosure is represented by either of the following or a pharmaceutically acceptable salt thereof:

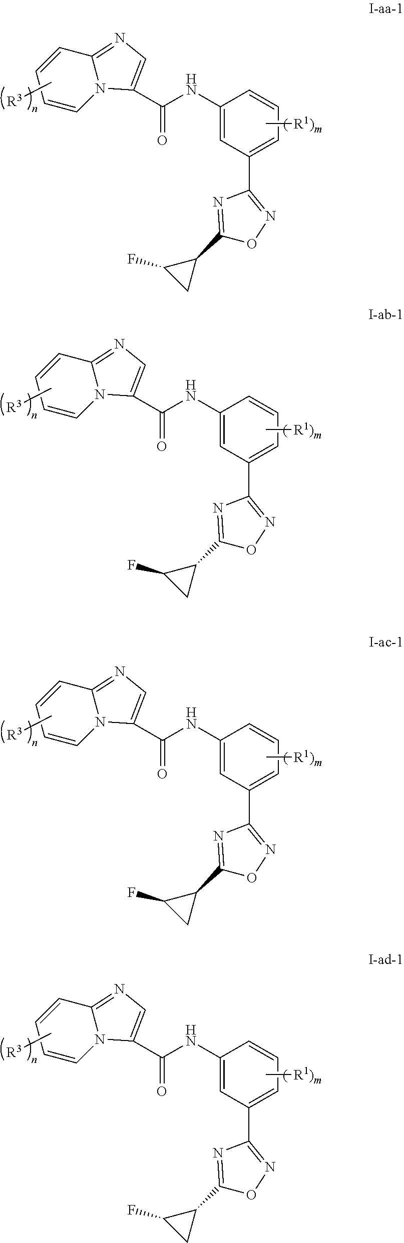

wherein R¹, R³, m, and n are as defined above and described herein.

In some embodiments, a compound of the present disclosure is represented by either of the following or a pharmaceutically acceptable salt thereof:

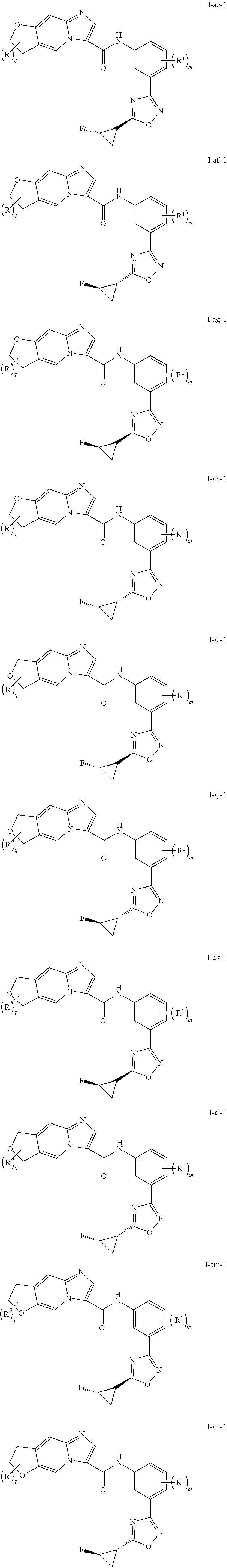

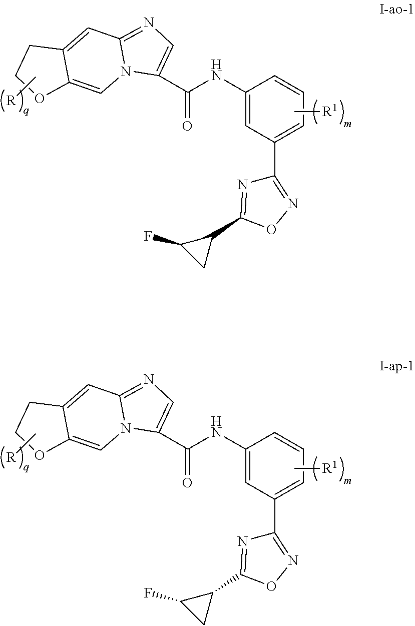

wherein R¹, R, m, and q are as defined above and described herein.

In some embodiments, a compound of the present disclosure is represented by any of the following or a pharmaceutically acceptable salt thereof:

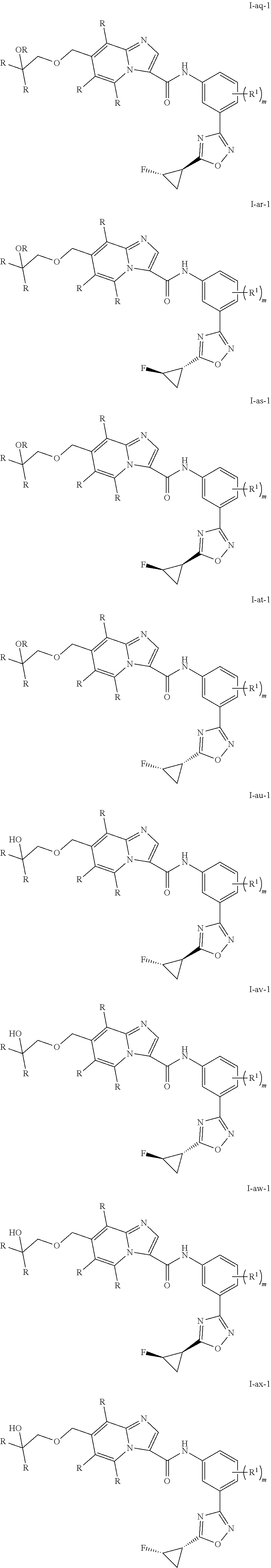

wherein R, R¹, and m are as defined above and described herein. In some embodiments, each R is independently selected from H, F, Cl, —CH₃, —CH₂F, —CHF₂ and —CF₃.

In some embodiments, a compound of the present disclosure is represented by any of the following or a pharmaceutically acceptable salt thereof:

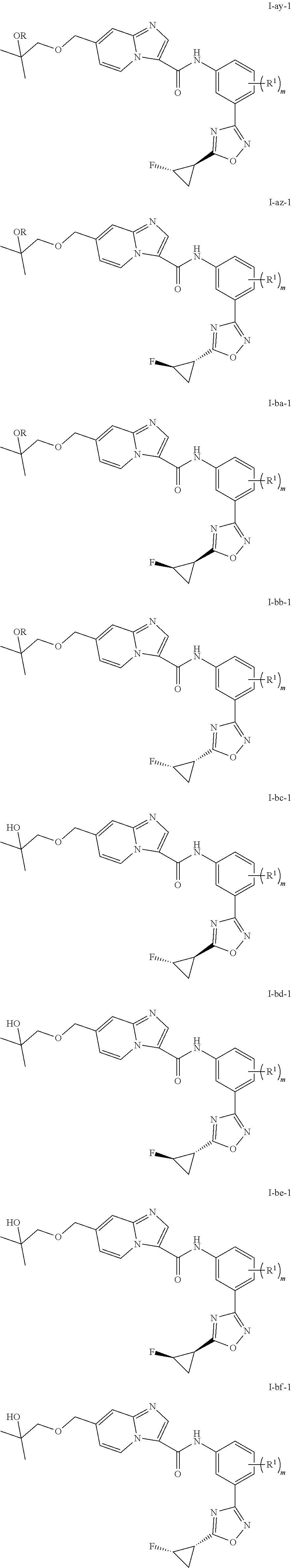

wherein R, R¹, and m are as defined above and described herein.

In some embodiments, a compound of the present disclosure is represented by any of the following or a pharmaceutically acceptable salt thereof:

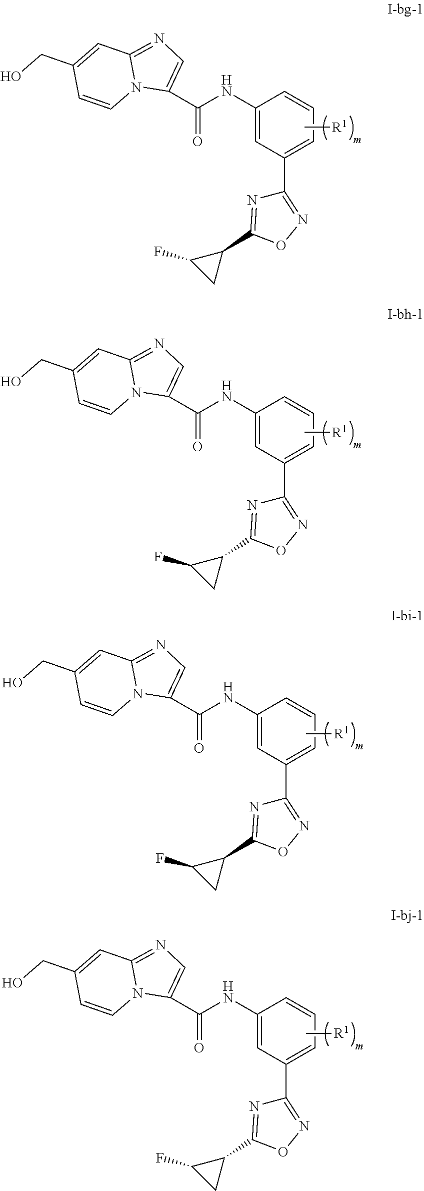

wherein R¹ and m are as defined above and described herein.

In some embodiments, a compound of the present disclosure is represented by any of the following or a pharmaceutically acceptable salt thereof:

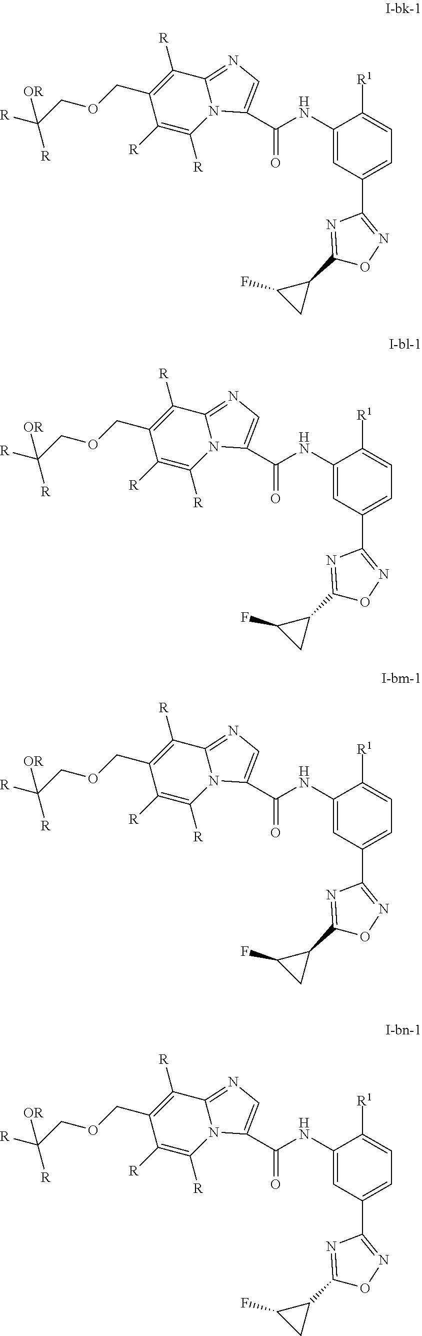

wherein R and R¹ are as defined above and described herein. In some embodiments, each R is independently selected from H, F, Cl, —CH₃, —CH₂F, —CHF₂ and —CF₃.

In some embodiments, a compound of the present disclosure is represented by any of the following or a pharmaceutically acceptable salt thereof:

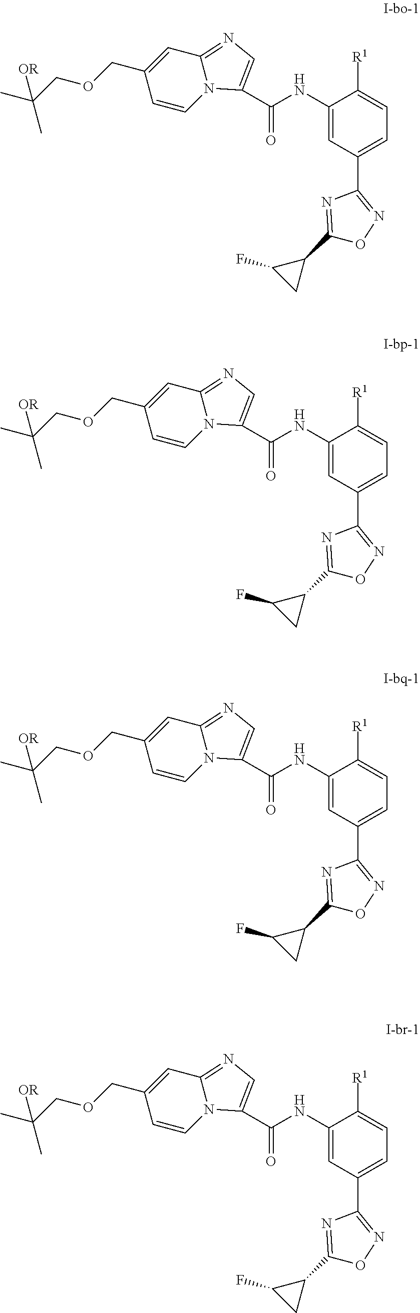

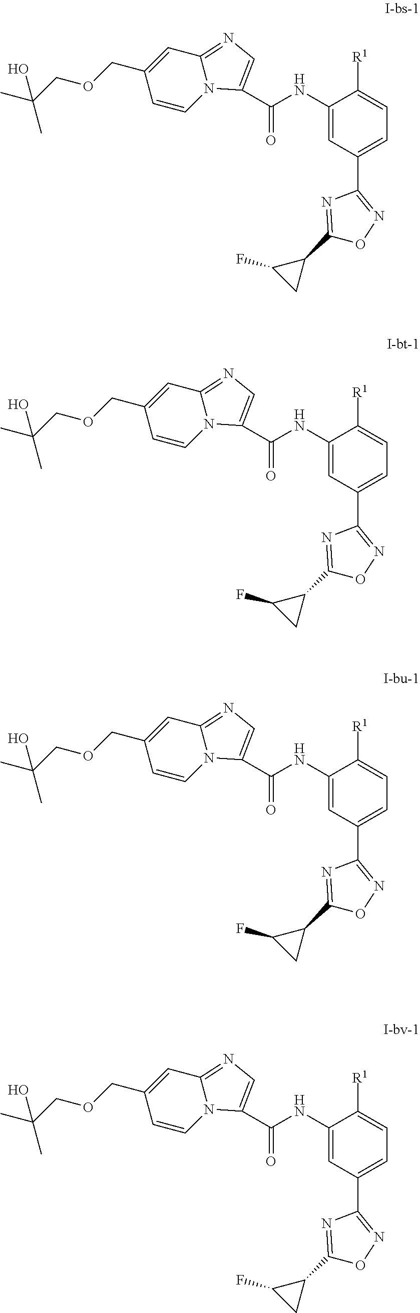

wherein R and R¹ are as defined above and described herein.

In some embodiments, a compound of the present disclosure is represented by any of the following or a pharmaceutically acceptable salt thereof:

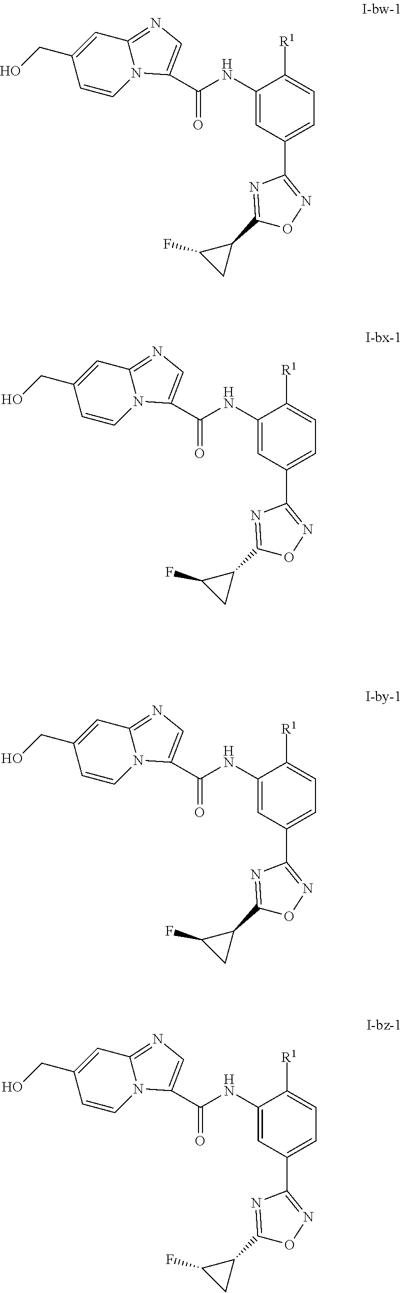

wherein R¹ is as defined above and described herein.

In some embodiments, a compound of the present disclosure is represented by either of the following or a pharmaceutically acceptable salt thereof:

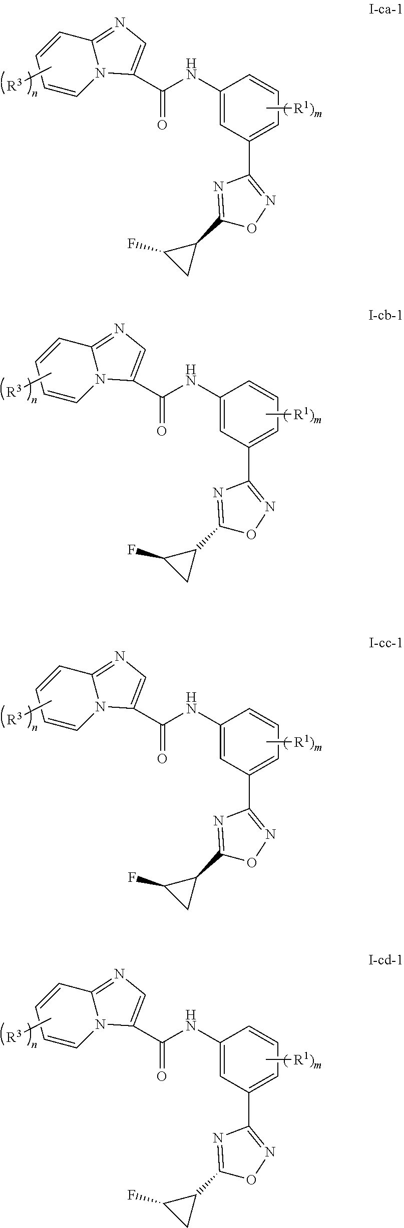

wherein R¹, R³, m and n are as defined above and described herein. In some such embodiments, n is 1 and R³ is H,

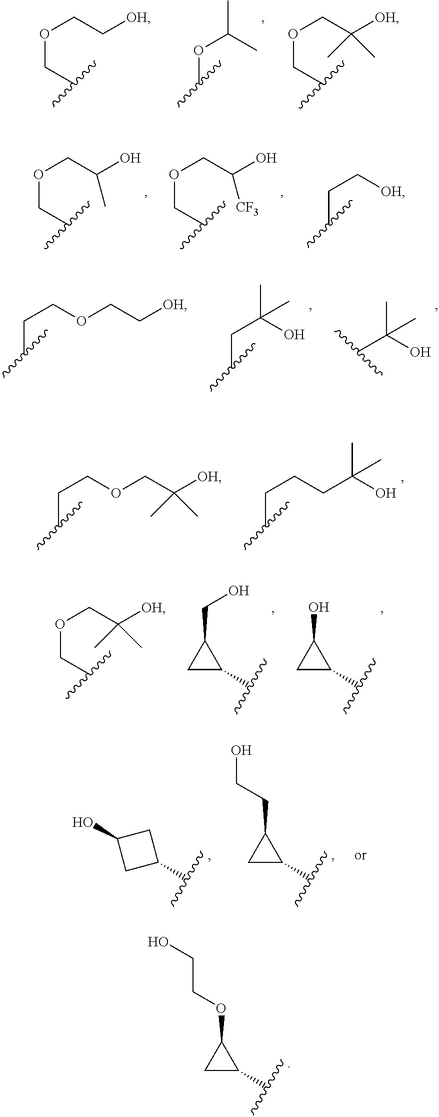

In some such embodiments, n is 2 and each R³ is independently H, F, Cl,

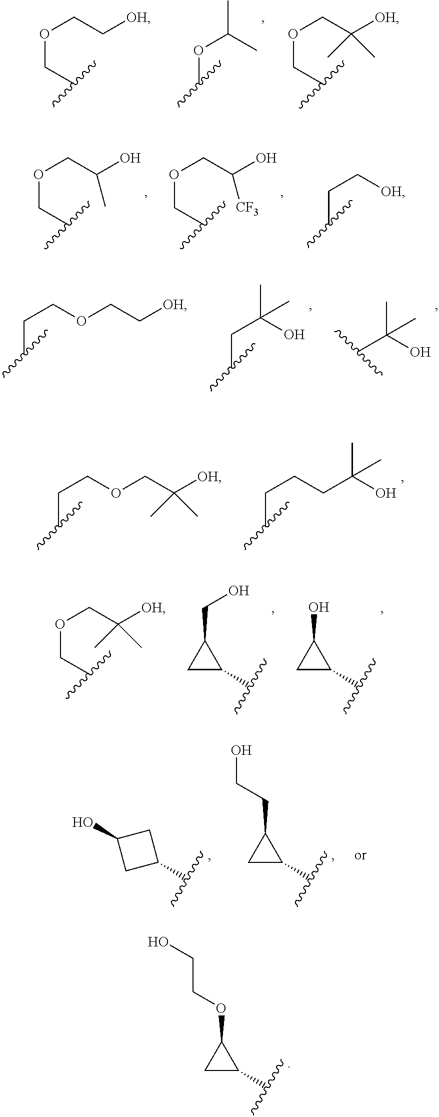

In some such embodiments, n is 2, one of R³ is selected from H, F and Cl, and second R³ is selected from

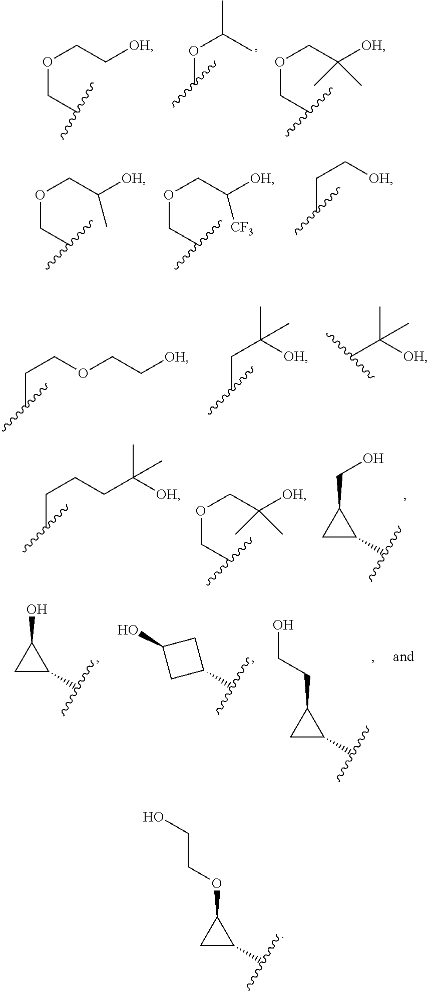

In some embodiments, a compound of the present disclosure is represented by either of the following or a pharmaceutically acceptable salt thereof:

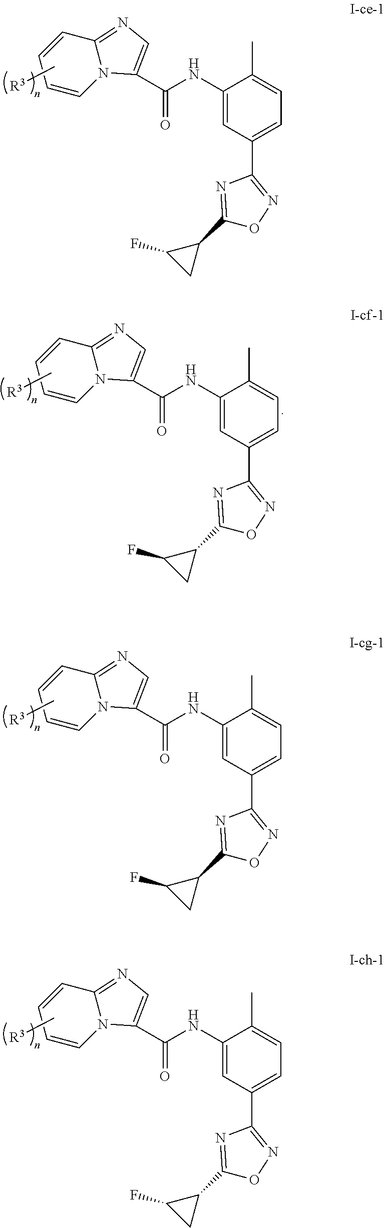

wherein R³ and n are as defined above and described herein. In some such embodiments, n is 1 and R³ is H,

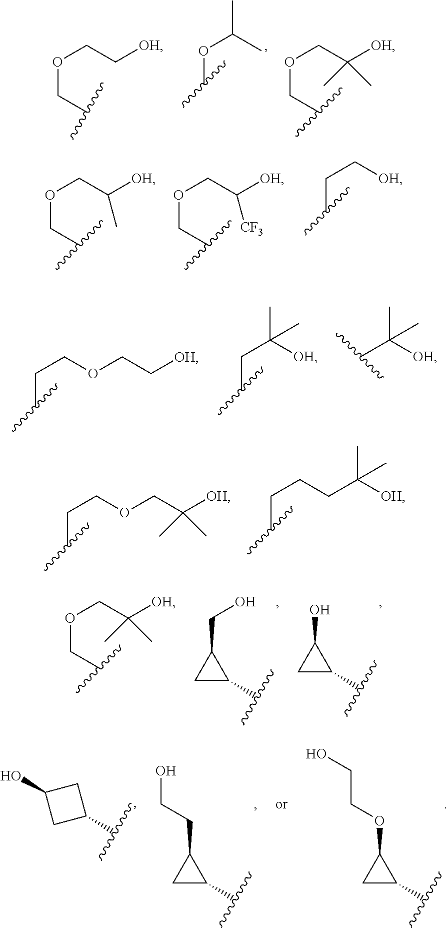

In some such embodiments, n is 2 and each R³ is independently H, F, Cl,

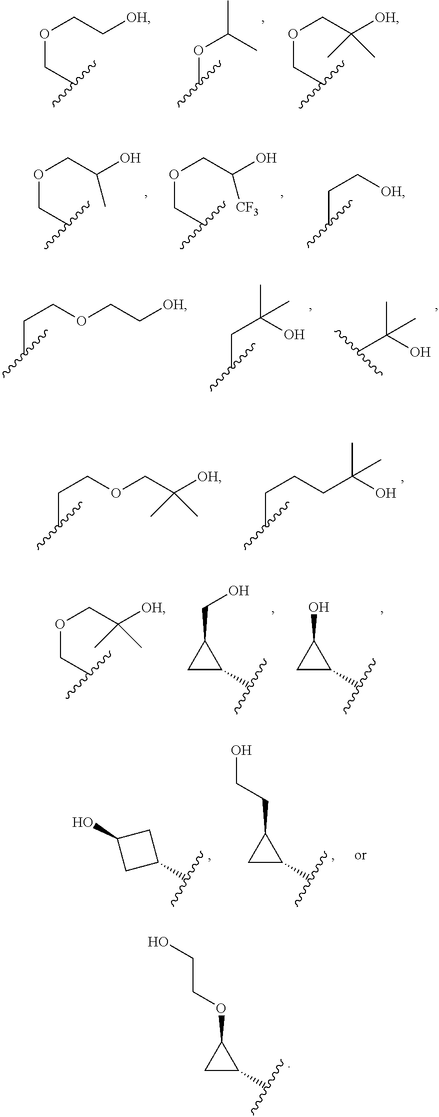

In some such embodiments, n is 2, one of R³ is selected from H, F and Cl, and second R³ is selected from

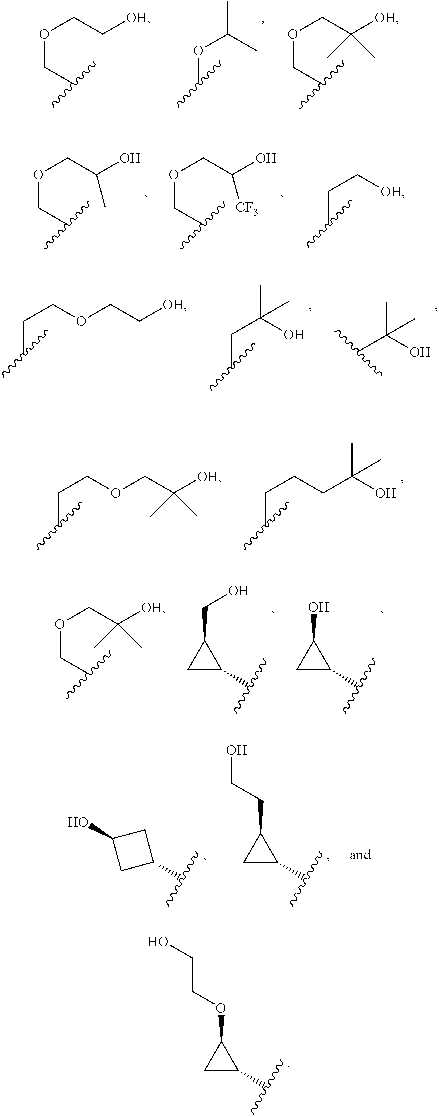

In some embodiments, a compound of the present disclosure is represented by any of the following or a pharmaceutically acceptable salt thereof:

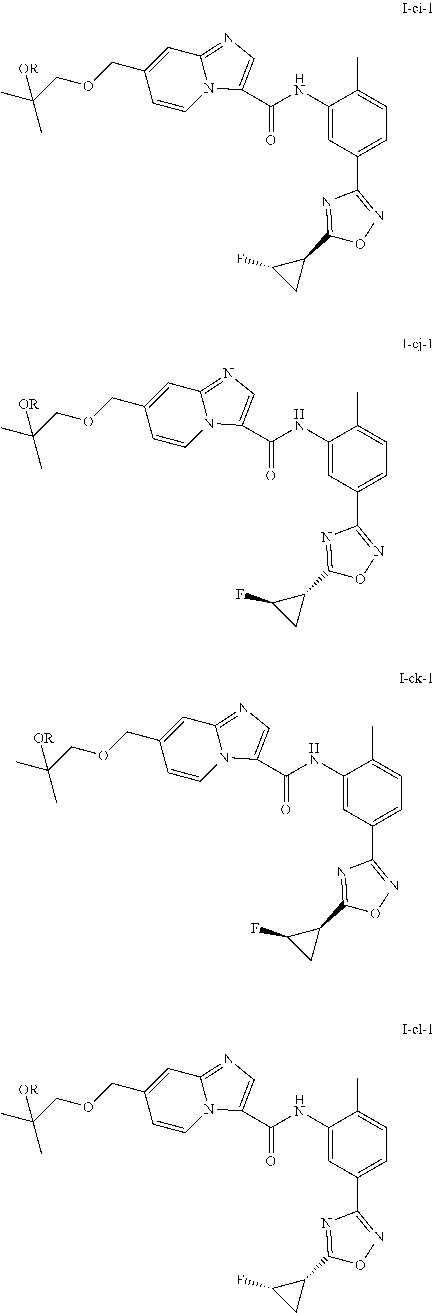

wherein R is as defined above and described herein.

In some embodiments, a compound of the present disclosure is represented by any of the following or a pharmaceutically acceptable salt thereof:

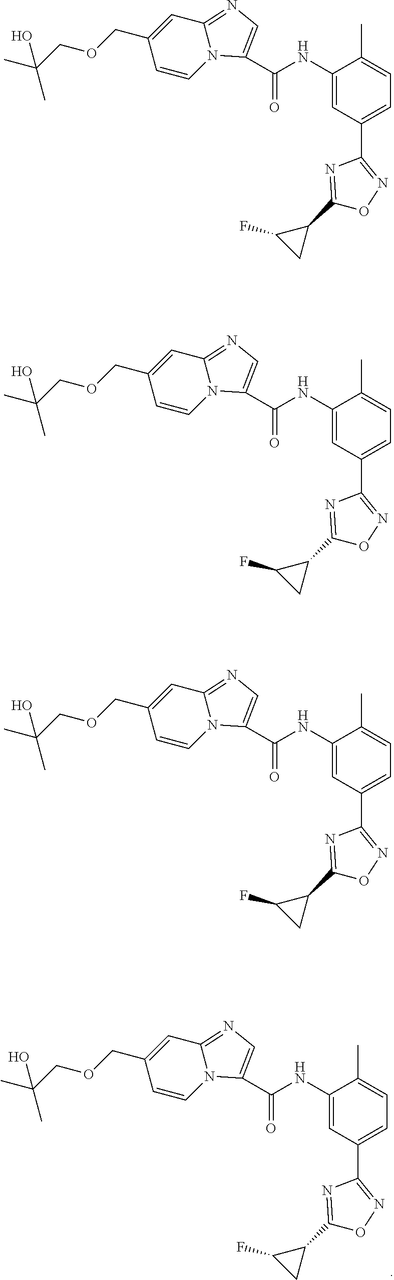 [SMILES: Cc1ccc(-c2noc([C@H]3C[C@@H]3F)n2)cc1NC(=O)c1cnc2cc(COCC(C)(C)O)ccn12 ; Cc1ccc(-c2noc([C@@H]3C[C@H]3F)n2)cc1NC(=O)c1cnc2cc(COCC(C)(C)O)ccn12 ; Cc1ccc(-c2noc([C@H]3C[C@H]3F)n2)cc1NC(=O)c1cnc2cc(COCC(C)(C)O)ccn12 ; Cc1ccc(-c2noc([C@@H]3C[C@@H]3F)n2)cc1NC(=O)c1cnc2cc(COCC(C)(C)O)ccn12]

In some embodiments, a compound of the present disclosure is represented by any of the following or a pharmaceutically acceptable salt thereof:

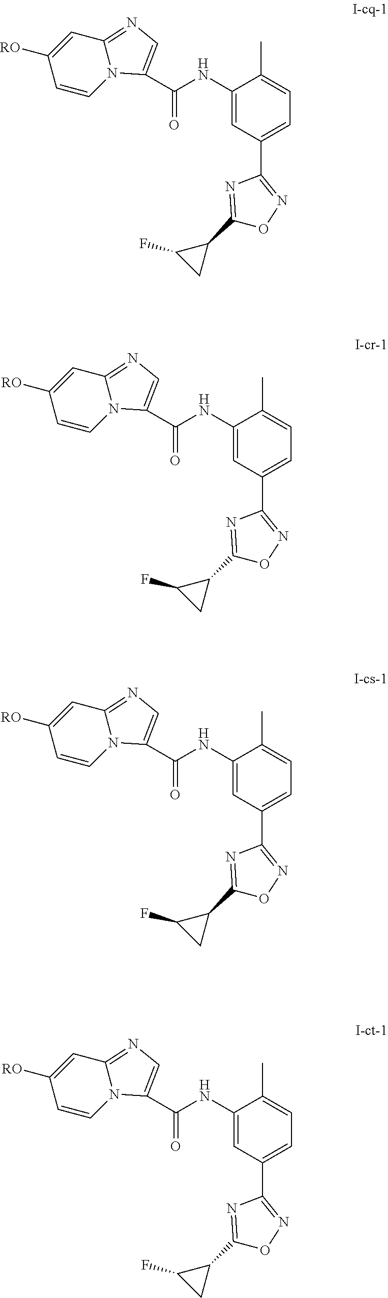

wherein R is as defined above and described herein. In some embodiments, R is i-Pr group.

In some embodiments, a compound of the present disclosure is represented by any of the following or a pharmaceutically acceptable salt thereof:

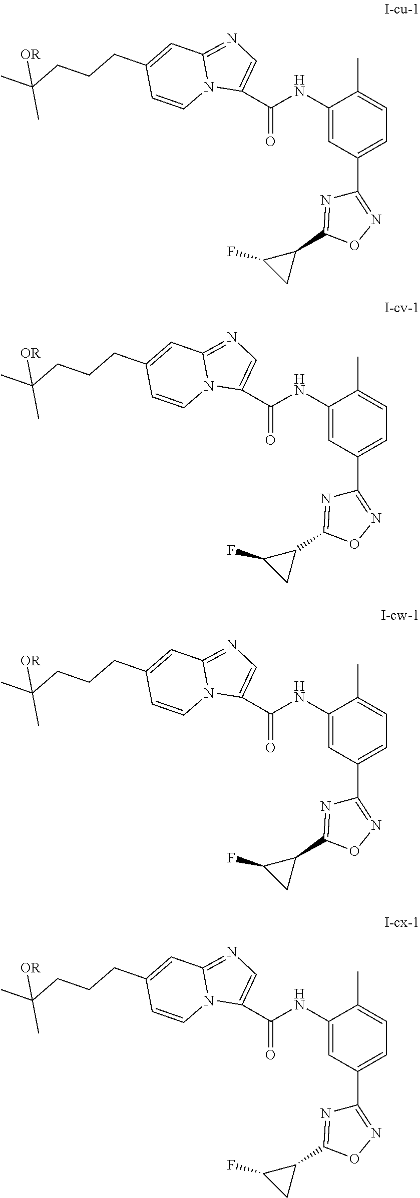

wherein R is as defined above and described herein.

In some embodiments, a compound of the present disclosure is represented by any of the following or a pharmaceutically acceptable salt thereof:

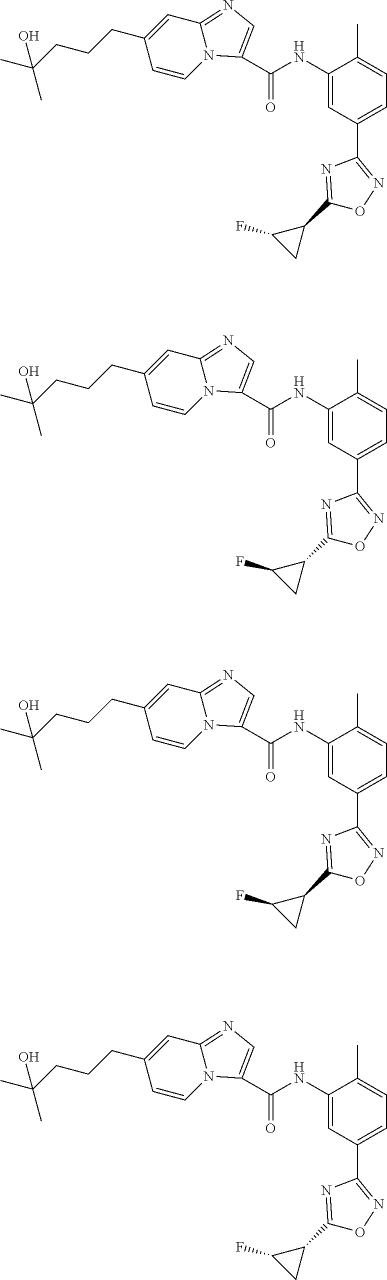 [SMILES: Cc1ccc(-c2noc([C@H]3C[C@@H]3F)n2)cc1NC(=O)c1cnc2cc(CCCC(C)(C)O)ccn12 ; Cc1ccc(-c2noc([C@@H]3C[C@H]3F)n2)cc1NC(=O)c1cnc2cc(CCCC(C)(C)O)ccn12 ; Cc1ccc(-c2noc([C@H]3C[C@H]3F)n2)cc1NC(=O)c1cnc2cc(CCCC(C)(C)O)ccn12 ; Cc1ccc(-c2noc([C@@H]3C[C@@H]3F)n2)cc1NC(=O)c1cnc2cc(CCCC(C)(C)O)ccn12]

In some embodiments, a compound of the present disclosure is represented by any of the following or a pharmaceutically acceptable salt thereof:

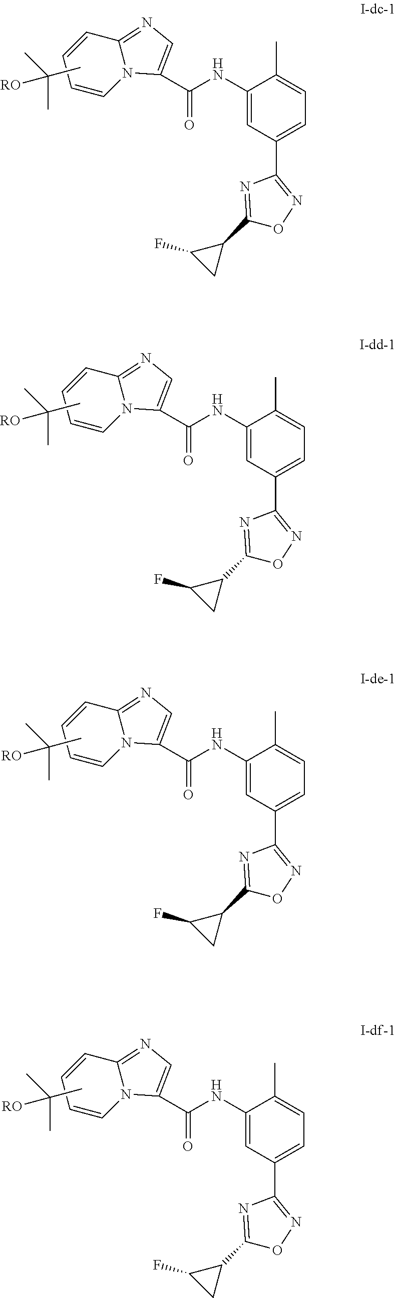

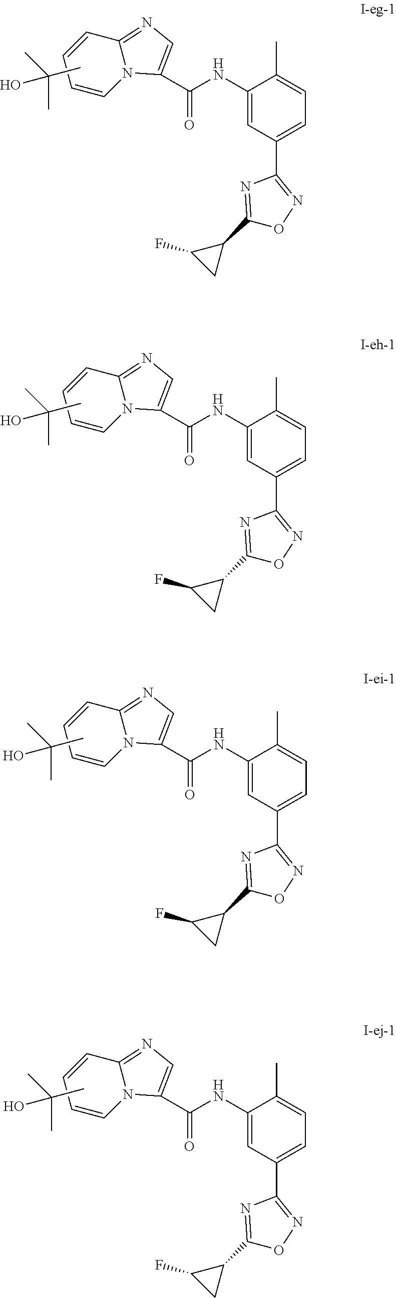

wherein R is as defined above and described herein.

In some embodiments, a compound of the present disclosure is represented by any of the following or a pharmaceutically acceptable salt thereof:

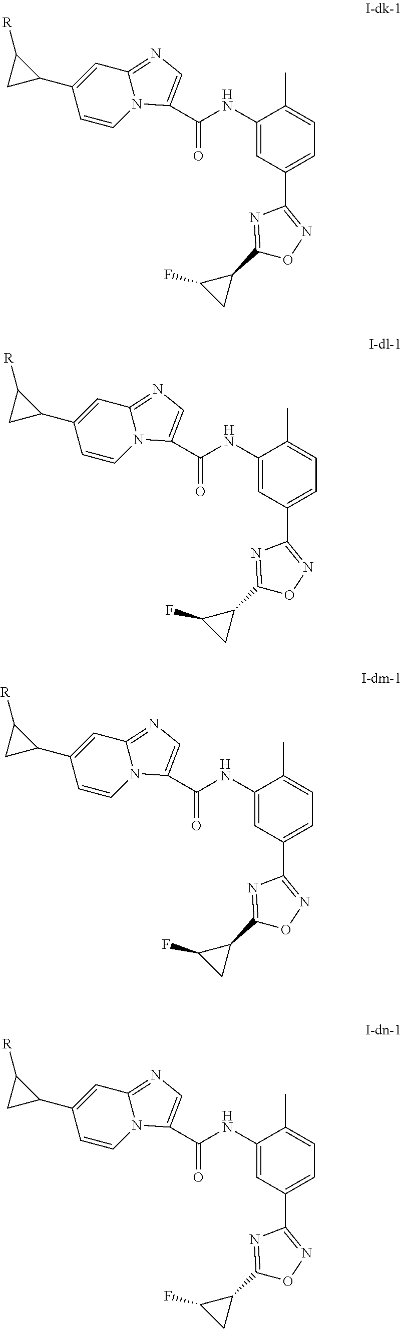

wherein R is as defined above and described herein. In some embodiments, R is —OH,

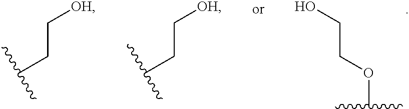

In some embodiments, a compound of the present disclosure is represented by any of the following or a pharmaceutically acceptable salt thereof:

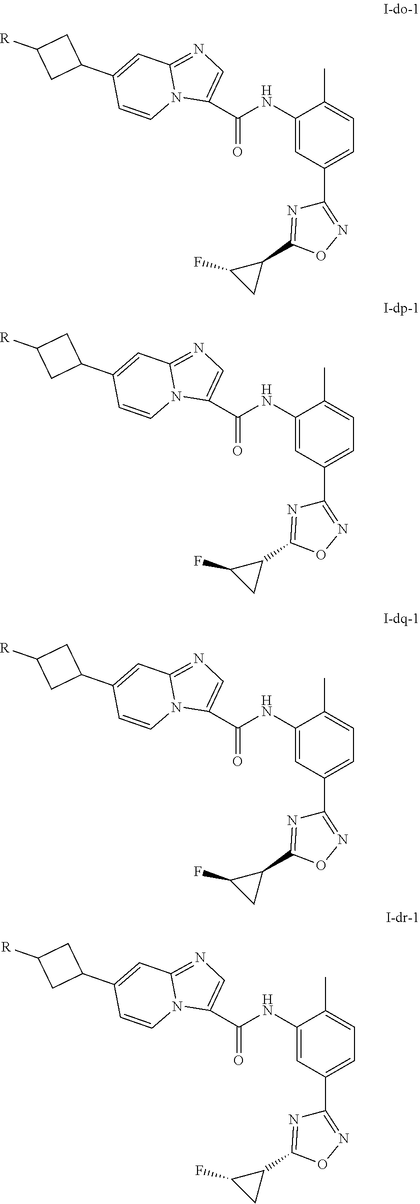

wherein R is as defined above and described herein. In some embodiments, R is —OH.

In some embodiments, a compound of the present disclosure is represented by any of the following or a pharmaceutically acceptable salt thereof:

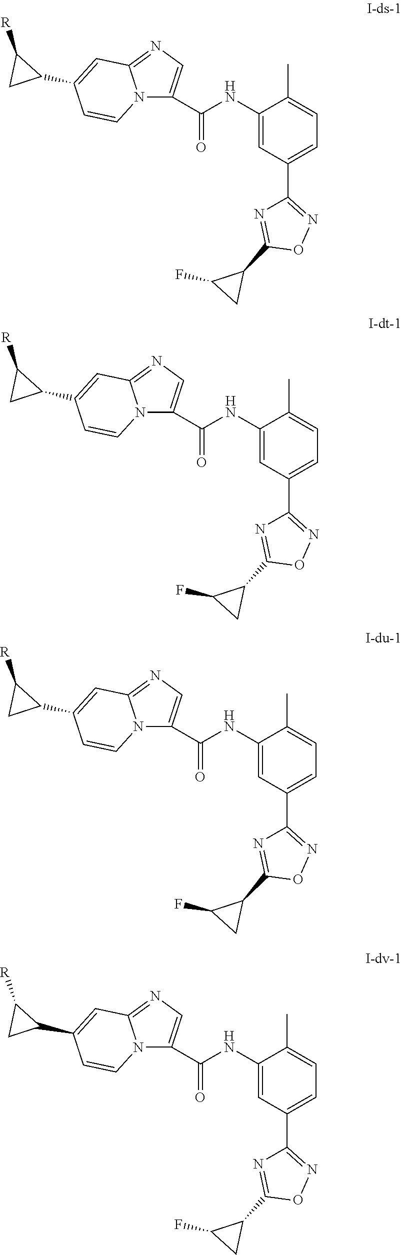

wherein R is as defined above and described herein. In some embodiments, R is —OH,

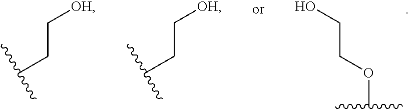

In some embodiments, a compound of the present disclosure is represented by any of the following or a pharmaceutically acceptable salt thereof:

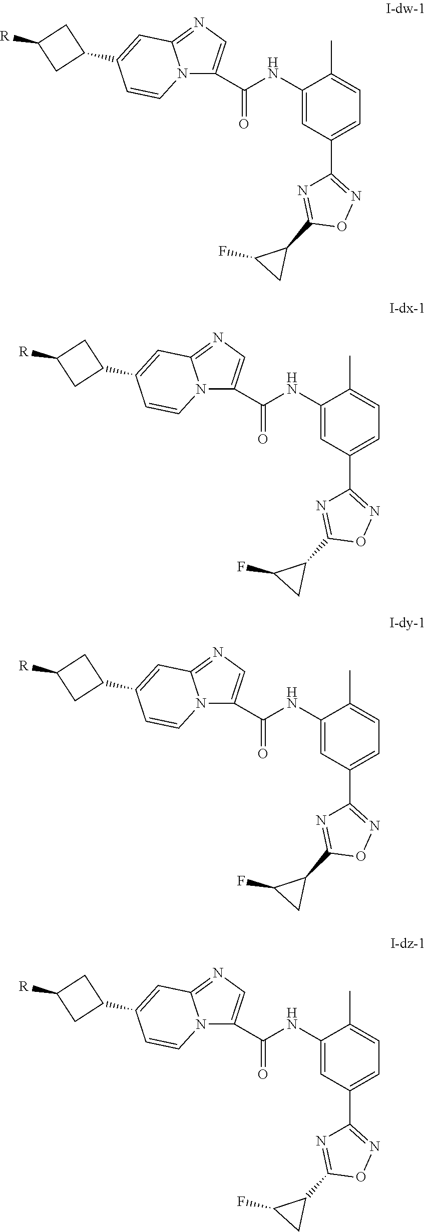

wherein R is as defined above and described herein. In some embodiments, R is —OH.

In some embodiments, a compound of the present disclosure is represented by any of the following or a pharmaceutically acceptable salt thereof:

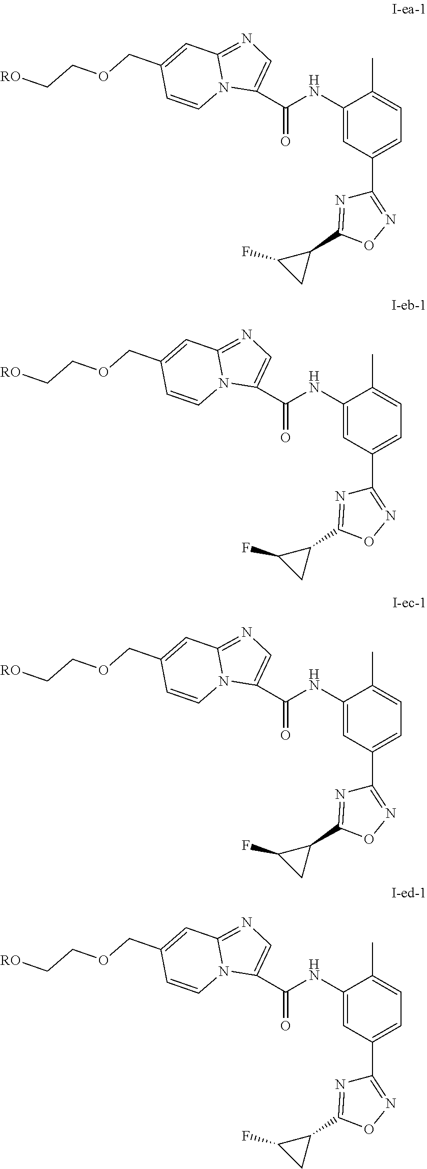

wherein R is as defined above and described herein.

In some embodiments, a compound of the present disclosure is represented by any of the following or a pharmaceutically acceptable salt thereof:

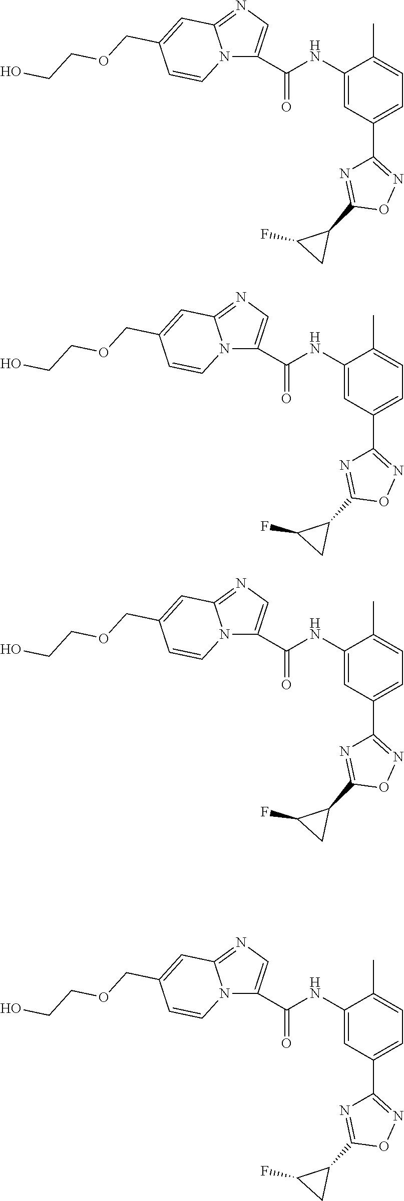 [SMILES: Cc1ccc(-c2noc([C@H]3C[C@@H]3F)n2)cc1NC(=O)c1cnc2cc(COCCO)ccn12 ; Cc1ccc(-c2noc([C@@H]3C[C@H]3F)n2)cc1NC(=O)c1cnc2cc(COCCO)ccn12 ; Cc1ccc(-c2noc([C@H]3C[C@H]3F)n2)cc1NC(=O)c1cnc2cc(COCCO)ccn12 ; Cc1ccc(-c2noc([C@@H]3C[C@@H]3F)n2)cc1NC(=O)c1cnc2cc(COCCO)ccn12]

In some embodiments, a compound of the present disclosure is represented by any of the following or a pharmaceutically acceptable salt thereof:

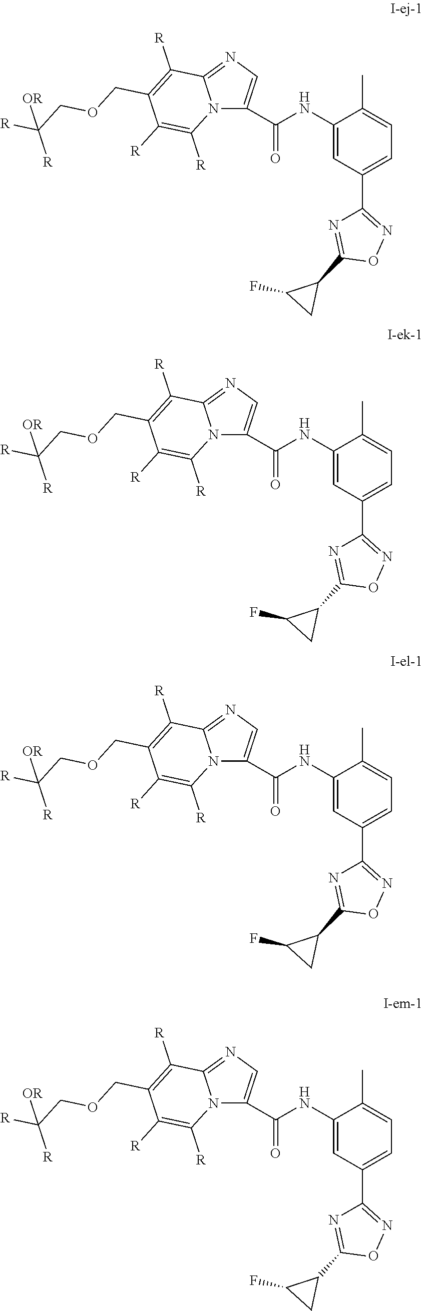

wherein R is as defined above and described herein. In some embodiments, each R is independently selected from H, F, Cl, —CH₃, —CH₂F, —CHF₂ and —CF₃.

In some embodiments, a compound of the present disclosure is represented by any of the following or a pharmaceutically acceptable salt thereof:

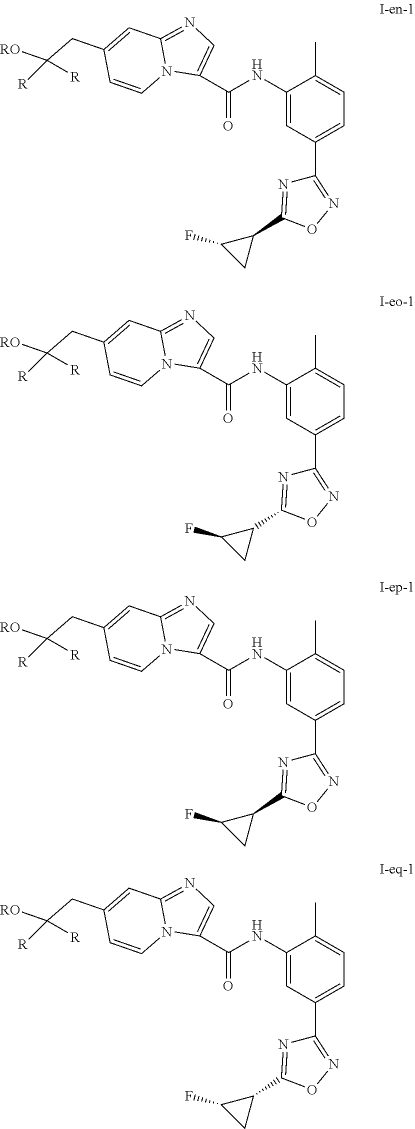

wherein R is as defined above and described herein. In some embodiments, each R is independently selected from H, —CH₃,

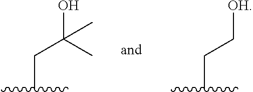

In some embodiments, a compound of the present disclosure is represented by any of the following or a pharmaceutically acceptable salt thereof:

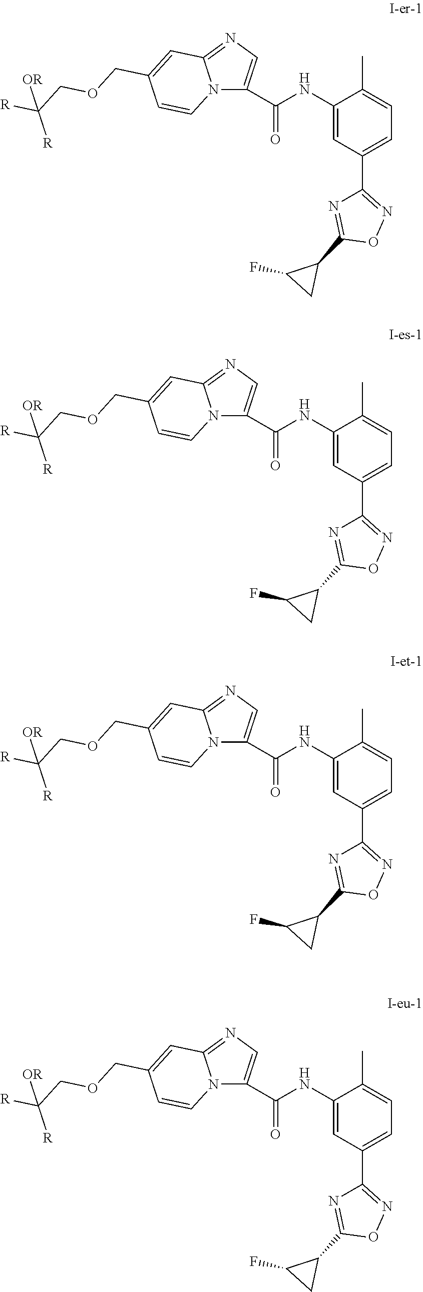

wherein R is as defined above and described herein. In some embodiments, each R is independently selected from H, —CH₃, —CH₂F, —CHF₂ and —CF₃.

In some embodiments, a compound of the present disclosure is represented by any of the following or a pharmaceutically acceptable salt thereof:

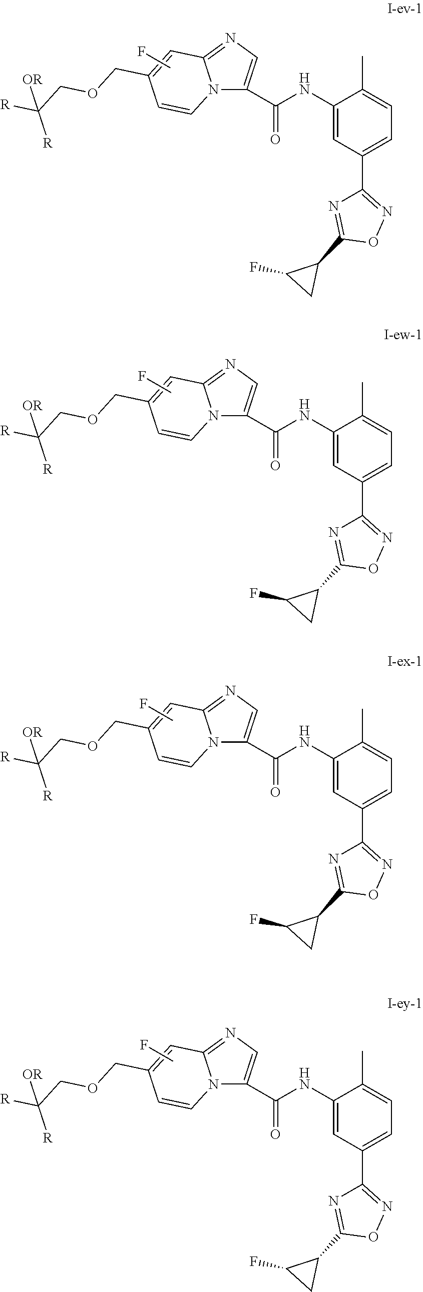

wherein R is as defined above and described herein. In some embodiments, each R is independently selected from H, —CH₃, —CH₂F, —CHF₂ and —CF₃.

In some embodiments, a compound of the present disclosure is represented by any of the following or a pharmaceutically acceptable salt thereof:

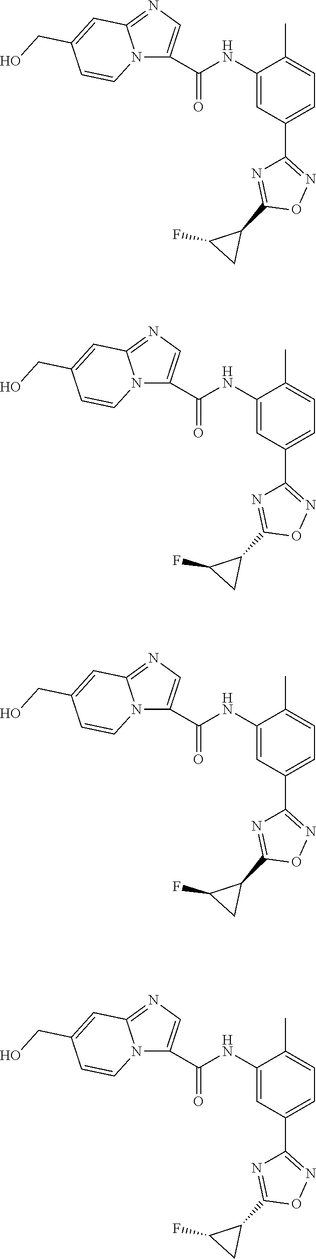 [SMILES: Cc1ccc(-c2noc([C@H]3C[C@@H]3F)n2)cc1NC(=O)c1cnc2cc(CO)ccn12 ; Cc1ccc(-c2noc([C@@H]3C[C@H]3F)n2)cc1NC(=O)c1cnc2cc(CO)ccn12 ; Cc1ccc(-c2noc([C@H]3C[C@H]3F)n2)cc1NC(=O)c1cnc2cc(CO)ccn12 ; Cc1ccc(-c2noc([C@@H]3C[C@@H]3F)n2)cc1NC(=O)c1cnc2cc(CO)ccn12]

In some embodiments, a compound of the present disclosure is represented by either of the following or a pharmaceutically acceptable salt thereof:

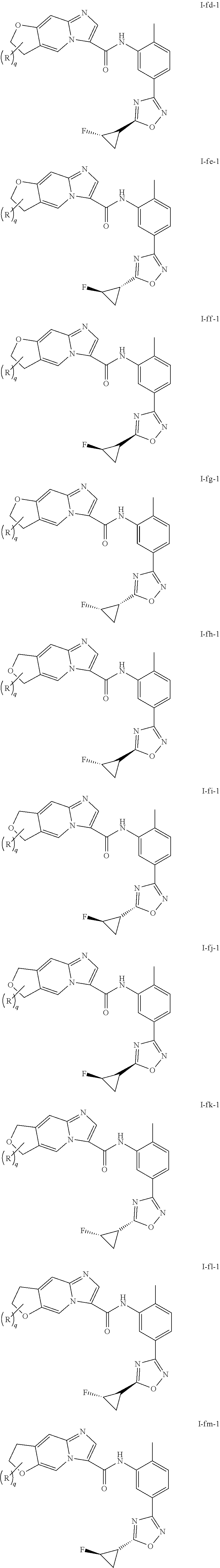

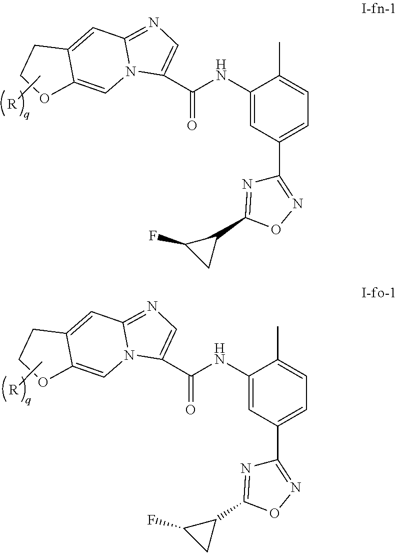

wherein R and q are as defined above and described herein. In some embodiments, q is 1. In some embodiments, q is 2. In some embodiments, R is Me. In some embodiments, the two R groups are present on the same carbon atom. In some embodiments, the two R groups are present on different carbon atoms. In some embodiments, R is Me, q is 2 and the two methyl groups are present on the same carbon atom.

In some embodiments, a compound of the present disclosure is represented by either of the following or a pharmaceutically acceptable salt thereof:

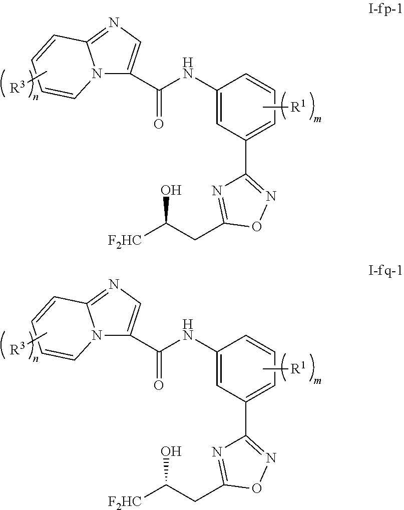

wherein R¹, R³, m and n are as defined above and described herein.

In some embodiments, a compound of the present disclosure is represented by either of the following or a pharmaceutically acceptable salt thereof:

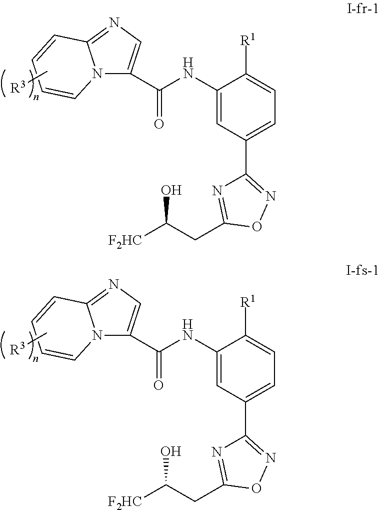

wherein R¹, R³, and n are as defined above and described herein.

In some embodiments, a compound of the present disclosure is represented by either of the following or a pharmaceutically acceptable salt thereof:

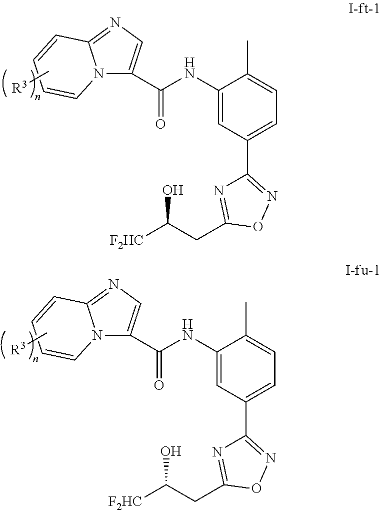

wherein R³ and n are as defined above and described herein. In some such embodiments, n is 0.

In some embodiments, a compound is of formulae I-a-1-I-fu-1 above, wherein n is 0. In some embodiments, a compound is of formulae I-a-1-I-fu-1 above, wherein n is 1.

In certain embodiments, the compound is a compound in Table 1. In certain embodiments, the compound is a compound in Table 1, or a pharmaceutically acceptable salt thereof. In certain embodiments, the compound is a compound selected from I-1 to I-446 in Table 1, or a pharmaceutically acceptable salt thereof. In certain embodiments, the compound is a compound selected from I-1 to I-446 in Table 1.

Lengthy table referenced here
US20240254119A1-20240801-T00001
Please refer to the end of the specification for access instructions.

### II. Therapeutic Applications

### C-Kit Kinase Mediated Diseases and Disorders

It is contemplated that compounds described herein, such those of Formula I, provide therapeutic benefits to subjects suffering from a c-kit kinase-mediated disease, disorder or condition. Accordingly, one aspect of the invention provides a method of treating a disorder associated with c-kit kinase in a subject. The method comprises administering a therapeutically effective amount of a compound described herein, such as a compound of Formula I, to a subject in need thereof to treat the disorder. In certain embodiments, the compound is a compound of any of Formulae I-a to I-fu, inclusive, as depicted herein and defined by the embodiments described above.

It is contemplated that compounds described herein, such those of Formula I-1, provide therapeutic benefits to subjects suffering from a c-kit kinase-mediated disease, disorder or condition. Accordingly, one aspect of the invention provides a method of treating a disorder associated with c-kit kinase in a subject. The method comprises administering a therapeutically effective amount of a compound described herein, such as a compound of Formula I-1, to a subject in need thereof to treat the disorder. In certain embodiments, the compound is a compound of any of Formulae I-a-1 to I-fu-1, inclusive, as depicted herein and defined by the embodiments described above.

In some embodiments, the c-kit kinase-mediated disease, disorder or condition is associated with wild-type c-kit kinase. In some embodiments, the c-kit kinase-mediated disease, disorder or condition is associated with mutant c-kit kinase.

In some embodiments, the kit mutation is selected from D419, D816Y, D816F, N822, V559, K558Q, I517P, Duplication 572-573, V559A, V559D, W557R, V560G, L576P, K642E, D820V, V560G, D52N, D816V, D816, V825A, E490K, W557R, V559A, V560Del, V560G, K642E, V654A, D816H, D820E, A829P, T417, Y418, D419, A502, K509I, V530I, F552C, A533D, V560, ITD, V559D, K704, N705, S715, 1748T, L773S, V8251, and D816N.

Non-limiting examples of the c-kit kinase-mediated disease include Acute Myeloid Leukemia, Mastocytosis, AMI-HMCI-cell line, GIST, Melanoma, Myeloproliferative Disease, Renal Cell Carcinoma, Papillary renal carcinoma, Sinonasal NK/Tcell Lymphoma, Thymic Carcinoma, Acute Lymphoblastic leukemia, Germ cell tumor, Acute Myelogenous Leukemia, and Extranodal NK/T cell lymphoma.

The method may be further characterized according to the c-kit kinase mediated disease or disorder that is to be treated in the patient. In some embodiments, the c-kit kinase mediated disease or disorder is a mast-cell associated disease, a respiratory disease, an inflammatory disorder, an autoimmune disorder, a metabolic disease, a fibrotic disease, a dermatological disease, an allergic disease, a cardiovascular disease, or a neurological disorder. In some embodiments, the c-kit kinase mediated disease or disorder is a mast-cell associated disease. In some embodiments, the c-kit kinase mediated disease or disorder is an inflammatory disorder. In some embodiments, the c-kit kinase mediated disease or disorder is an autoimmune disorder. In some embodiments, the c-kit kinase mediated disease or disorder is a metabolic disease. In some embodiments, the c-kit kinase mediated disease or disorder is a fibrotic disease. In some embodiments, the c-kit kinase mediated disease or disorder is a dermatological disease. In some embodiments, the c-kit kinase mediated disease or disorder is an allergic disease. In some embodiments, the c-kit kinase mediated disease or disorder is a cardiovascular disease. In some embodiments, the c-kit kinase mediated disease or disorder is a neurological disorder.

In some embodiments, the disease or disorder is asthma, allergic rhinitis, pulmonary arterial hypertension (PAH), primary pulmonary hypertension (PPH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, food allergy, chronic rhinosinusitis, type I diabetes, type II diabetes, systemic sclerosis, allergic keratoconjunctivitis, vernal keratoconjunctivitis, Crohn's disease, or systemic and cutaneous lupus erythematosus and dermatomyositis. In some embodiments, the disease or disorder is asthma. In some embodiments, the disease or disorder is allergic rhinitis. In some embodiments, the disease or disorder is pulmonary arterial hypertension (PAH). In some embodiments, the disease or disorder is primary pulmonary hypertension (PPH). In some embodiments, the disease or disorder is pulmonary fibrosis. In some embodiments, the disease or disorder is hepatic fibrosis. In some embodiments, the disease or disorder is cardiac fibrosis. In some embodiments, the disease or disorder is scleroderma. In some embodiments, the disease or disorder is irritable bowel syndrome (IBS). In some embodiments, the disease or disorder is inflammatory bowel disease (IBD). In some embodiments, the disease or disorder is urticaria. In some embodiments, the disease or disorder is dermatosis. In some embodiments, the disease or disorder is atopic dermatitis. In some embodiments, the disease or disorder is allergic contact dermatitis. In some embodiments, the disease or disorder is rheumatoid arthritis. In some embodiments, the disease or disorder is multiple sclerosis. In some embodiments, the disease or disorder is melanoma. In some embodiments, the disease or disorder is a gastrointestinal stromal tumor. In some embodiments, the disease or disorder is a mast cell tumor. In some embodiments, the disease or disorder is mastocytosis. In some embodiments, the disease or disorder is anaphylactic syndrome. In some embodiments, the disease or disorder is food allergy. In some embodiments, the disease or disorder is chronic rhinosinusitis. In some embodiments, the disease or disorder is type I diabetes. In some embodiments, the disease or disorder is type II diabetes. In some embodiments, the disease or disorder is systemic sclerosis. In some embodiments, the disease or disorder is allergic keratoconjunctivitis. In some embodiments, the disease or disorder is vernal keratoconjunctivitis. In some embodiments, the disease or disorder is Crohn's disease. In some embodiments, the disease or disorder is systemic and cutaneous lupus erythematosus and dermatomyositis.

In some embodiments, the c-kit mediated disease or disorder is urticaria. In some embodiments, the urticaria is chronic urticaria. In some embodiments, the urticaria is inducible urticaria. In some embodiments, the urticaria is chronic and inducible urticaria. In some embodiments, the urticaria is spontaneous urticaria. In some embodiments, the urticaria is chronic and spontaneous urticaria. In some embodiments, the urticaria is cold inducible urticaria. In some embodiments, the urticaria is heat inducible urticaria (also referred to as cholinergic urticaria (ChoIU)). In some embodiments, the urticaria is friction inducible urticaria (also referred to as symptomatic dermographism). In some embodiments, the urticaria is delayed pressure urticaria (DPU). In some embodiments, the urticaria is solar urticaria. In some embodiments, the urticaria is vibratory angioedema. In some embodiments, the urticaria is aquagenic urticaria. In some embodiments, the urticaria is contact urticaria. In some embodiments, the urticaria is any of those described above and herein and is chronic.

In some embodiments, the disease or disorder is mast cell gastrointestinal disease, prurigo nodularis, allergic conjunctivitis, eosinophilic esophagitis, mast cell activation syndrome, eosinophilic gastritis and/or eosinophilic duodenitis (EG/EoD), ulcerative colitis, eosinophilic gastritis (EG), or eosinophilic colitis (EC). In some embodiments, the disease or disorder is mast cell gastrointestinal disease. In some embodiments, the disease or disorder is prurigo nodularis. In some embodiments, the disease or disorder is allergic conjunctivitis. In some such embodiments, the allergic conjunctivitis is seasonal conjunctivitis. In some such embodiments, the allergic conjunctivitis is perennial conjunctivitis. In some embodiments, the disease or disorder is eosinophilic esophagitis. In some embodiments, the disease or disorder is mast cell activation syndrome. In some embodiments, the disease or disorder is eosinophilic gastritis and/or eosinophilic duodenitis (EG/EoD). In some embodiments, the disease or disorder is ulcerative colitis.

In some embodiments, the present invention provides a method for treating a c-kit kinase mediated disorder comprising the step of administering to a patient in need thereof a therapeutically effective compound of the present invention, or pharmaceutically acceptable composition thereof.

In some aspects and embodiments, provided herein are methods of treating, reducing the severity of, delaying the onset of, or inhibiting the progress of a disease or disorder, or one or more symptoms thereof of a disease or disorder characterized by or associated with increased c-kit kinase, comprising the step of administering to a patient in need thereof a therapeutically effective amount of a compound of the present invention, or pharmaceutically acceptable composition thereof. In some aspects and embodiments, provided herein are methods of treating, reducing the severity of, delaying the onset of, or inhibiting the progress of a disease or disorder, or one or more symptoms thereof of a disease or disorder in which inhibition of c-kit kinase activity is beneficial, comprising the step of administering to a patient in need thereof a therapeutically effective amount of a compound of the present invention, or pharmaceutically acceptable composition thereof.

As used herein, the terms “increased,” “elevated,” or “enhanced,” are used interchangeably and encompass any measurable increase in a biological function and/or biological activity and/or a concentration. For example, an increase can be by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, relative to a control or baseline amount of a function, or activity, or concentration.

In certain embodiments, the subject is a human. In certain embodiments, the subject is an adult human. In certain embodiments, the subject is a pediatric human. In certain embodiments, the subject is a companion animal. In certain embodiments, the subject is a canine, feline, or equine.

Another aspect of the invention provides for the use of a compound described herein (such as a compound of Formula I, or other compounds in Section I) in the manufacture of a medicament. In certain embodiments, the medicament is for treating a disorder described herein, such as a c-kit kinase mediated disorder.

Another aspect of the invention provides for the use of a compound described herein (such as a compound of Formula I, or other compounds in Section I) for treating a medical disorder, such as a medical disorder described herein (for example, a c-kit kinase mediated disorder).

Another aspect of the invention provides for the use of a compound described herein (such as a compound of Formula I-1, or other compounds in Section I) in the manufacture of a medicament. In certain embodiments, the medicament is for treating a disorder described herein, such as a c-kit kinase mediated disorder.

Another aspect of the invention provides for the use of a compound described herein (such as a compound of Formula I-1, or other compounds in Section I) for treating a medical disorder, such as a medical disorder described herein (for example, a c-kit kinase mediated disorder).

### II. Pharmaceutical Compositions and Dosing Considerations

As used herein, the terms “combination,” “combined,” and related terms refer to the simultaneous or sequential administration of therapeutic agents in accordance with this disclosure. For example, a described compound may be administered with another therapeutic agent simultaneously or sequentially in separate unit dosage forms or together in a single unit dosage form. Accordingly, the present disclosure provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

When the compounds of this disclosure are administered in combination therapies with other agents, they may be administered sequentially or concurrently to the patient. Alternatively, pharmaceutical or prophylactic compositions according to this disclosure may comprise a combination of a compound of Formula I and another therapeutic or prophylactic agent. Additional therapeutic agents that are normally administered to treat a particular disease or condition may be referred to as “agents appropriate for the disease, or condition, being treated.”

When the compounds of this disclosure are administered in combination therapies with other agents, they may be administered sequentially or concurrently to the patient. Alternatively, pharmaceutical or prophylactic compositions according to this disclosure may comprise a combination of a compound of Formula I-1 and another therapeutic or prophylactic agent. Additional therapeutic agents that are normally administered to treat a particular disease or condition may be referred to as “agents appropriate for the disease, or condition, being treated.”

In some embodiments, the subject method includes administering a therapeutically effective amount of one or more additional active agents. By combination therapy is meant that a c-kit inhibiting compound can be used in a combination with another therapeutic agent to treat a single disease or condition. In some embodiments, a compound of the present disclosure is administered concurrently with the administration of another therapeutic agent, which can be administered as a component of a composition including the compound of the present disclosure or as a component of a different composition.

The subject compounds can be administered in combination with other therapeutic agents in a variety of therapeutic applications. Therapeutic applications of interest for combination therapy include those applications in which activity of a target c-kit kinase is the cause or a compounding factor in disease progression. As such, the subject compounds find use in combination therapies in which the inhibition of a target c-kit kinase in the subject is desired. The compounds utilized in the compositions and methods of this disclosure may also be modified by appending appropriate functionalities to enhance selective biological properties. Such modifications are known in the art and include those, which increase biological penetration into a given biological system (e.g., blood, lymphatic system, or central nervous system), increase oral availability, increase solubility to allow administration by injection, alter metabolism and/or alter rate of excretion.

The term “treatment” is used interchangeably herein with the term “therapeutic method” and refers to both 1) therapeutic treatments or measures that cure, slow down, lessen symptoms of, and/or halt progression of a diagnosed pathologic conditions, disease or disorder, and 2) and prophylactic/preventative measures. Those in need of treatment may include individuals already having a particular medical disease or disorder as well as those who may ultimately acquire the disorder (i.e., those at risk or needing preventive measures).

The term “subject” as used herein refers to any individual or patient to which the subject methods are performed. Generally, the subject is human, although as will be appreciated by those in the art, the subject may be an animal.

The terms “therapeutically effective amount”, “effective dose”, “therapeutically effective dose”, “effective amount,” or the like refer to the amount of a subject compound that will elicit the biological or medical response in a tissue, system, animal or human that is being sought by administering said compound. Generally, the response is either amelioration of symptoms in a patient or a desired biological outcome. In some embodiments, such amount should be sufficient to inhibit a c-kit kinase.

Unless specified otherwise, the term “about” refers to within +10% of the stated value. The invention encompasses embodiments where the value is within +9%, +8%, +7%, +6%, +5%, +4%, +3%, +2%, or +1% of the stated value.

In some embodiments, an effective amount of a c-kit inhibiting compound is an amount that ranges from about 50 ng/ml to 50 pg/ml (e.g., from about 50 ng/ml to 40 pg/ml, from about 30 ng/ml to 20 pg/ml, from about 50 ng/ml to 10 μg/ml, from about 50 ng/ml to 1 μg/ml, from about 50 ng/ml to 800 ng/ml, from about 50 ng/ml to 700 ng/ml, from about 50 ng/ml to 600 ng/ml, from about 50 ng/ml to 500 ng/ml, from about 50 ng/ml to 400 ng/ml, from about 60 ng/ml to 400 ng/ml, from about 70 ng/ml to 300 ng/ml, from about 60 ng/ml to 100 ng/ml, from about 65 ng/ml to 85 ng/ml, from about 70 ng/ml to 90 ng/ml, from about 200 ng/ml to 900 ng/ml, from about 200 ng/ml to 800 ng/ml, from about 200 ng/ml to 700 ng/ml, from about 200 ng/ml to 600 ng/ml, from about 200 ng/ml to 500 ng/ml, from about 200 ng/ml to 400 ng/ml, or from about 200 ng/ml to about ng/ml).

In some embodiments, an effective amount of a c-kit inhibiting compound is an amount that ranges from about 10 pg to 100 mg, e.g., from about 10 pg to 50 pg, from about 50 pg to 150 pg, from about 150 pg to 250 pg, from about 250 pg to 500 pg, from about 500 pg to 750 pg, from about 750 pg to 1 ng, from about 1 ng to 10 ng, from about 10 ng to 50 ng, from about 50 ng to 150 ng, from about 150 ng to 250 ng, from about 250 ng to 500 ng, from about 500 ng to 750 ng, from about 750 ng to 1 mg, from about 1 pg to 10 pg, from about 10 pg to 50 pg, from about 50 pg to 150 pg, from about 150 pg to 250 pg, from about 250 pg to 500 pg, from about 500 pg to 750 pg, from about 750 pg to 1 mg, from about 1 mg to 50 mg, from about 1 mg to 100 mg, or from about 50 mg to 100 mg. The amount can be a single dose amount or can be a total daily amount. The total daily amount can range from about 10 pg to 100 mg, or can range from about 100 mg to 500 mg, or can range from about 500 mg to 1000 mg.

Also disclosed herein are pharmaceutical compositions including compounds as disclosed herein e.g., compounds of Formula I and pharmaceutically acceptable salts thereof.

Also disclosed herein are pharmaceutical compositions including compounds as disclosed herein e.g., compounds of Formula I-1 and pharmaceutically acceptable salts thereof.

The term “pharmaceutically acceptable carrier” refers to a non-toxic carrier that may be administered to a patient, together with a compound of this disclosure, and which does not destroy the pharmacological activity thereof. Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In pharmaceutical compositions comprising only the compounds described herein as the active component, methods for administering these compositions may additionally comprise the step of administering to the subject an additional agent or therapy. Such therapies include, but are not limited to, an anemia therapy, a diabetes therapy, a hypertension therapy, a cholesterol therapy, neuropharmacologic drugs, drugs modulating cardiovascular function, drugs modulating inflammation, immune function, production of blood cells, hormones and antagonists, drugs affecting gastrointestinal function, chemotherapeutics of microbial diseases, and/or chemotherapeutics of neoplastic disease. Other pharmacological therapies can include any other drug or biologic found in any drug class. For example, other drug classes can comprise allergy/cold/ENT therapies, analgesics, anesthetics, anti-inflammatories, antimicrobials, antivirals, asthma/pulmonary therapies, cardiovascular therapies, dermatology therapies, endocrine/metabolic therapies, gastrointestinal therapies, cancer therapies, immunology therapies, neurologic therapies, ophthalmic therapies, psychiatric therapies or rheumatologic therapies. Other examples of agents or therapies that can be administered with the compounds described herein include a matrix metalloprotease inhibitor, a lipoxygenase inhibitor, a cytokine antagonist, an immunosuppressant, a cytokine, a growth factor, an immunomodulator, a prostaglandin or an anti-vascular hyperproliferation compound.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology).

### Pharmaceutically Acceptable Compositions

The compounds and compositions, according to the method of the present disclosure, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the disclosure are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the disclosure are administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

All features of each of the aspects of the disclosure apply to all other aspects mutatis mutandis. Each of the references referred to herein, including but not limited to patents, patent applications and journal articles, is incorporated by reference herein as though fully set forth in its entirety.

In order that the disclosure described herein may be more fully understood, the following examples are set forth. It should be understood that these examples are for illustrative purposes only and are not to be construed as limiting this disclosure in any manner.

### Enumerated Embodiments

Embodiment 1. A compound represented by Formula I-1:

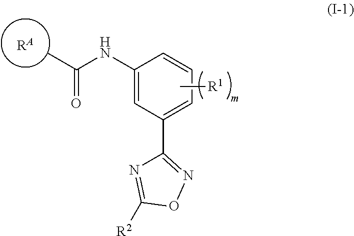

or a pharmaceutically acceptable salt thereof, wherein:
R¹ represents independently for each occurrence halogen, —CN, C₁₋₆ alkyl, or C₁₋₆ haloalkyl;
R² is C₁₋₆ aliphatic, phenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; or L¹-R⁴, wherein R² is substituted with p occurrences of R⁶;
L¹ is a C₁₋₂ bivalent saturated straight or branched hydrocarbon chain wherein one methylene unit of the chain is optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R⁴ is a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur;
R⁶ represents independently for each occurrence oxo, halogen, C₁₋₆ aliphatic, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or optionally substituted phenyl;
R^{A} is of any of the following structures:

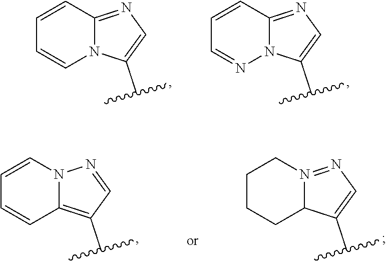

each of which is substituted by n occurrences of R³;
R³ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, -L²-R⁵, or an optionally substituted group selected from C₁₋₆ aliphatic, C₁₋₆ haloaliphatic, phenyl, naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; each of which is substituted with r instances of R; or:
two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur, substituted with r instances of R;
L² represents independently for each occurrence a C₁₋₆ bivalent saturated or unsaturated, straight or branched hydrocarbon chain wherein one, two, or three methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂— or -Cy-;
Cy represents independently for each occurrence phenyl, a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
R⁵ represents independently for each occurrence hydrogen, OR, C₁₋₆ aliphatic, C₁₋₆ haloaliphatic, or phenyl fused to a 5-6 membered saturated heterocyclic ring having 1-2 heteroatoms independently selected from nitrogen, oxygen, and sulfur; each R is independently hydrogen, —CN, halogen, or an optionally substituted group selected from C₁₋₆ aliphatic; C₁₋₆ haloaliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur;
m is 0, 1, 2, 3, or 4;
n is 0, 1, 2, 3, 4, or 5;
p is 0, 1, 2, 3, 4, or 5; and
r is 0, 1, 2, 3, 4, or 5.

Embodiment 2. The compound of Embodiment 1, wherein the compound is a compound of Formula I-1.

Embodiment 3. The compound of either of Embodiments 1-2, wherein at least one R¹ is halogen.

Embodiment 4. The compound of any one of Embodiments 1-3, wherein at least one R¹ is C₁₋₆ alkyl.

Embodiment 5. The compound of any one of Embodiments 1-4, wherein at least one R¹ is methyl.

Embodiment 6. The compound of either of Embodiments 1-5, wherein m is 0.

Embodiment 7. The compound of any one of Embodiments 1-5, wherein m is 1.

Embodiment 8. The compound of any one of Embodiments 1-5, wherein m is 2.

Embodiment 9. The compound of any one of Embodiments 1-5, wherein m is 3.

Embodiment 10. The compound of any one of Embodiments 1-5, wherein m is 4.

Embodiment 11. The compound of any one of Embodiments 1-5, wherein the compound is represented by one of the following or a pharmaceutically acceptable salt thereof:

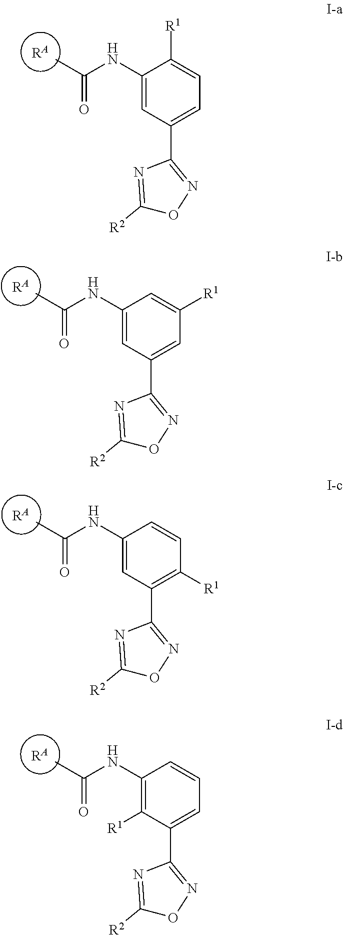

Embodiment 12. The compound of any one of Embodiments 1-5, wherein the compound is represented by one of the following or a pharmaceutically acceptable salt thereof:

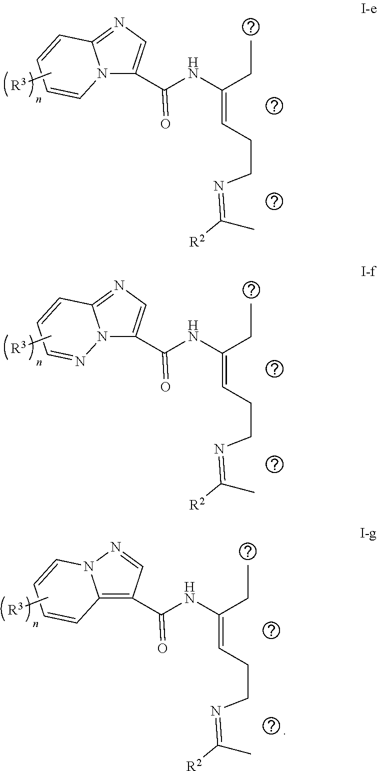

Embodiment 13. The compound of Embodiment 1, wherein the compound is represented by one of the following or a pharmaceutically acceptable salt thereof:

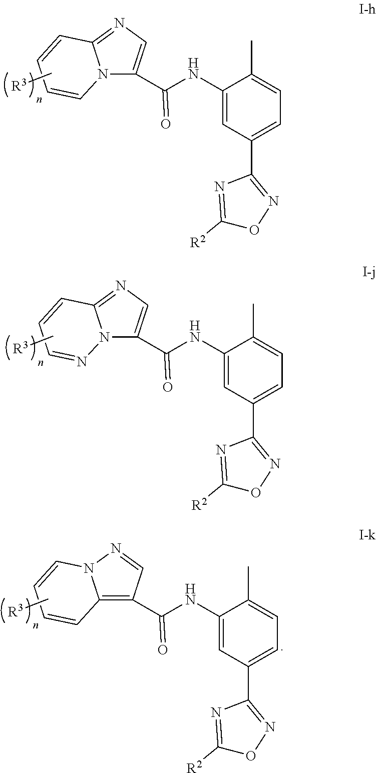

Embodiment 14. The compound of any one of Embodiments 1-13, wherein n is 0.

Embodiment 15. The compound of any one of Embodiments 1-13, wherein n is 1.

Embodiment 16. The compound of any one of Embodiments 1-13, wherein n is 2.

Embodiment 17. The compound of any one of Embodiments 1-13, wherein n is 3.

Embodiment 18. The compound of any one of Embodiments 1-13, wherein n is 4.

Embodiment 19. The compound of any one of Embodiments 1-13, wherein n is 5.

Embodiment 20. The compound of Embodiment 1, wherein the compound is represented by one of the following or a pharmaceutically acceptable salt thereof:

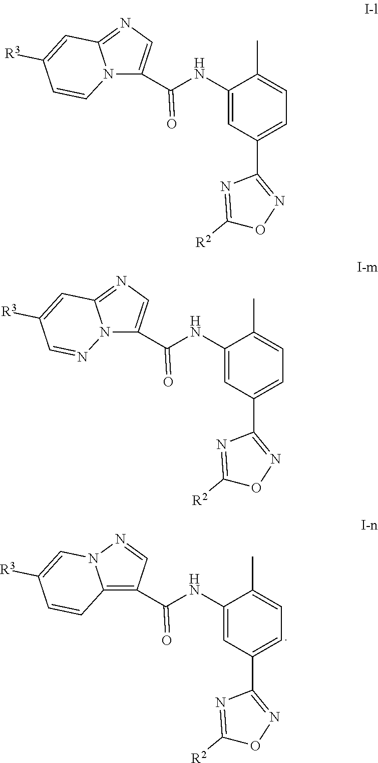

Embodiment 21. The compound of Embodiment 1, wherein the compound is represented by one of the following or a pharmaceutically acceptable salt thereof:

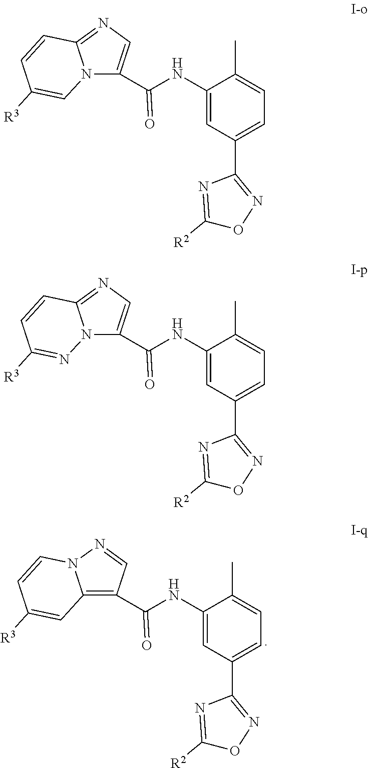

Embodiment 22. The compound of Embodiment 1, wherein the compound is represented by one of the following or a pharmaceutically acceptable salt thereof:

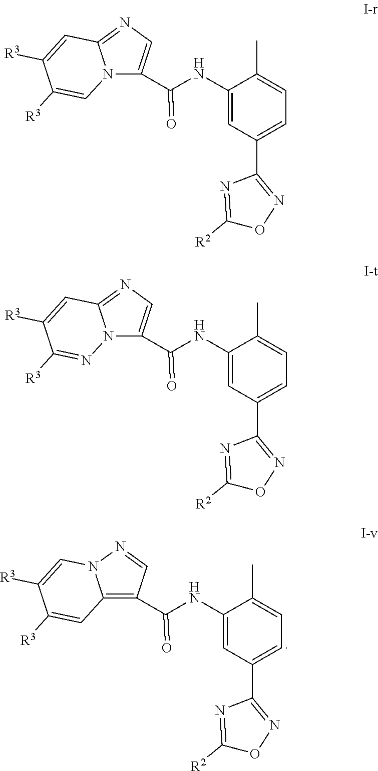

Embodiment 23. The compound of any one of Embodiments 1-13 or 15-22, wherein at least one R³ is halogen.

Embodiment 24. The compound of any one of Embodiments 1-13 or 15-22, wherein at least one R³ is fluoro.

Embodiment 25. The compound of any one of Embodiments 1-13 or 15-22, wherein at least one R³ is chloro.

Embodiment 26. The compound of any one of Embodiments 1-13 or 15-22, wherein at least one R³ is bromo.

Embodiment 27. The compound of any one of Embodiments 1-13 or 15-22, wherein at least one R³ is cyano.

Embodiment 28. The compound of any one of Embodiments 1-13 or 15-22, wherein at least one R³ is —OR.

Embodiment 29. The compound of Embodiment 28, wherein at least one R³ is —OR, wherein R is C₁₋₆ alkyl.

Embodiment 30. The compound of Embodiment 29, wherein at least one R³ is —OR, wherein R is methyl, ethyl, or propyl.

Embodiment 31. The compound of Embodiment 30, wherein at least one R³ is —OR, wherein R is methyl.

Embodiment 32. The compound of Embodiment 30, wherein at least one R³ is —OR, wherein R is ethyl.

Embodiment 33. The compound of Embodiment 30, wherein at least one R³ is —OR, wherein R is propyl.

Embodiment 34. The compound of any one of Embodiments 1-13 or 15-22, wherein at least one R³ is —OCR₃, wherein at least one R is fluoro.

Embodiment 35. The compound of any one of Embodiments 1-13 or 15-22, wherein at least one R³ is —NR₂.

Embodiment 36. The compound of Embodiment 35, wherein at least one R³ is —NR₂, wherein at least one R is hydrogen.

Embodiment 37. The compound of Embodiment 35, wherein at least one R³ is —NR₂, wherein at least one R is methyl or ethyl.

Embodiment 38. The compound of Embodiment 35, wherein at least one R³ is —NR₂, wherein at least one R is optionally substituted phenyl.

Embodiment 39. The compound of Embodiment 35, wherein the two R groups on the same nitrogen are taken together with the nitrogen to form an optionally substituted 4-7 membered monocyclic saturated ring having 1-3 heteroatoms independently selected from nitrogen, oxygen, and sulfur.

Embodiment 40. The compound of any one of Embodiments 1-13 or 15-22, wherein at least one R³ is —N(R)S(O)₂R.

Embodiment 41. The compound of Embodiment 40, wherein each R is independently hydrogen, C₁₋₆ alkyl, C₃₋₆ cycloalkyl, naphthalenyl, or a 5-membered heteroaryl ring having one, two, or three heteroatoms independently selected from nitrogen, oxygen, or sulfur.

Embodiment 42. The compound of any one of Embodiments 1-13 or 15-22, wherein at least one R³ is -L²-R⁵.

Embodiment 43. The compound of Embodiment 42, wherein one, two, or three methylene units of L² are independently replaced by —O— or -Cy-.

Embodiment 44. The compound of Embodiment 42, wherein one, two, or three methylene units of L² are independently replaced by —N(R)— or -Cy-.

Embodiment 45. The compound of any one of Embodiments 1-13 or 15-22, wherein at least one R³ is a 3-7 membered saturated or partially unsaturated monocyclic heterocyclic ring having 1-2 heteroatoms independently selected from nitrogen, oxygen, and sulfur.

Embodiment 46. The compound of Embodiment 45, wherein at least one R³ is oxetane.

Embodiment 47. The compound of any one of Embodiments 1-13 or 15-22, wherein at least one R³ is —CF₃, —CF₂H, or —CFH₂.

Embodiment 48. The compound of any one of Embodiments 1-13 or 15-22, wherein each R³ is independently selected from:

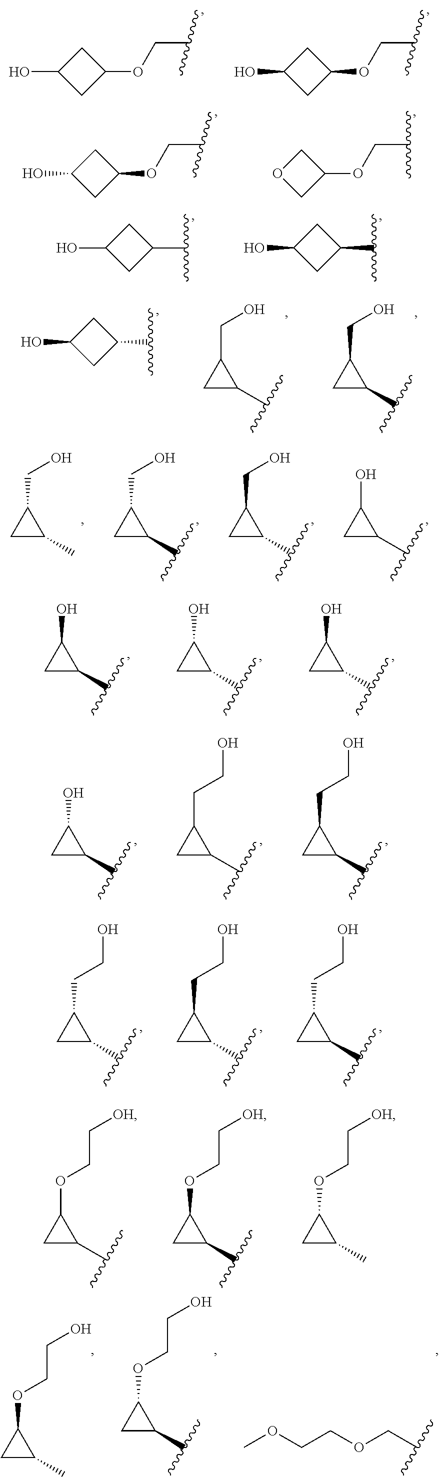

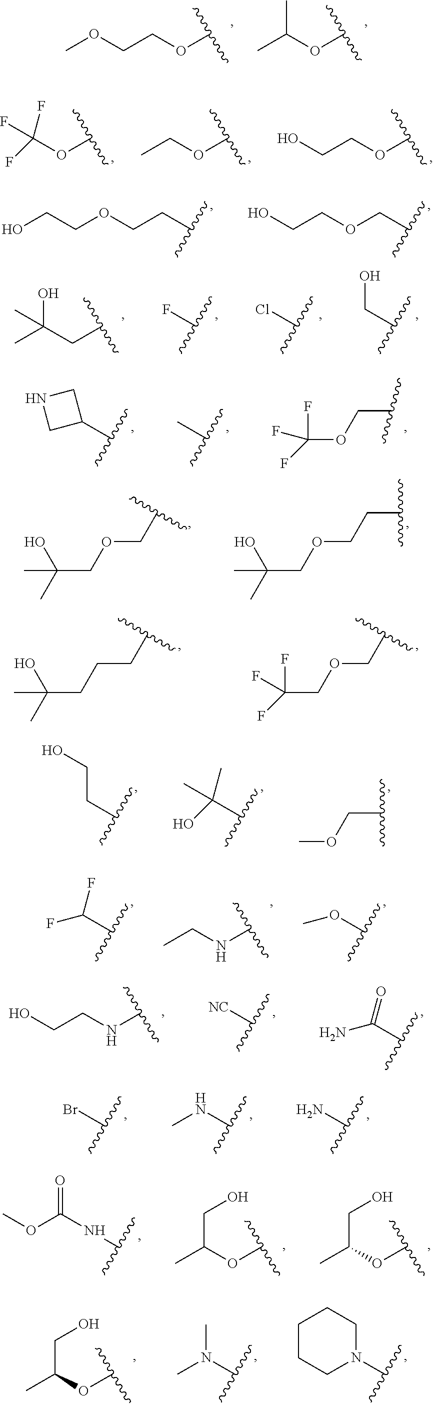

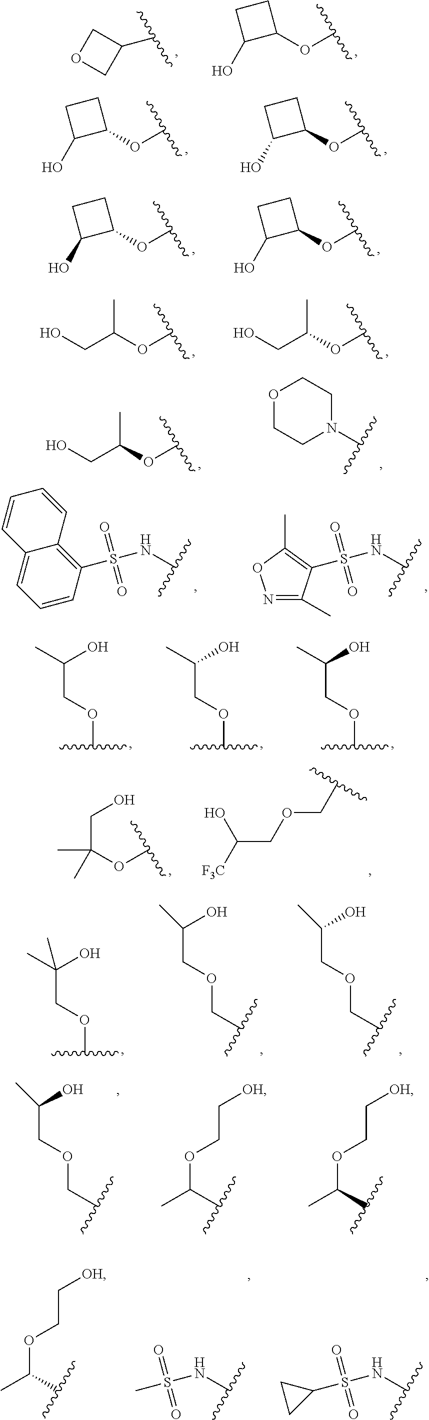

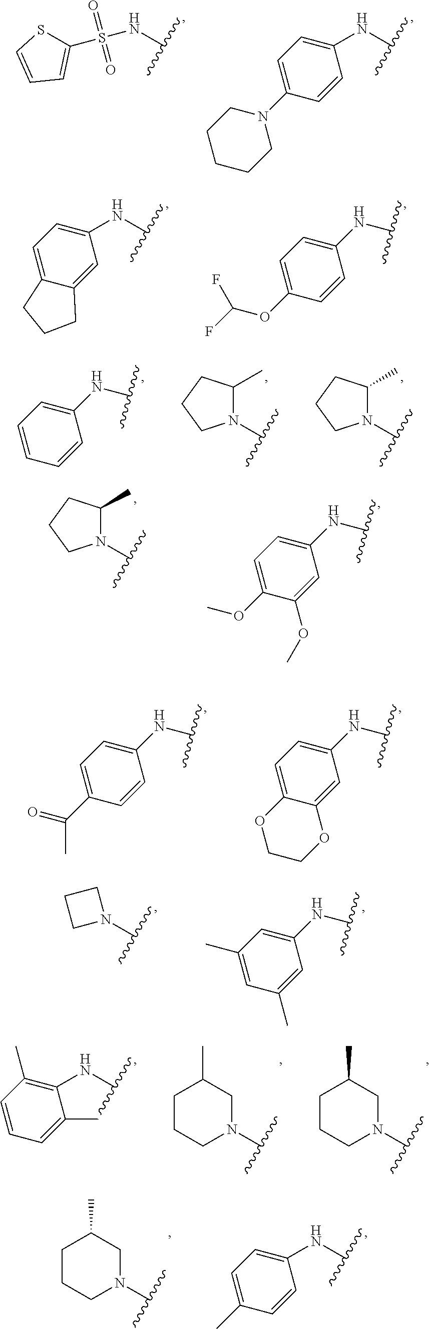

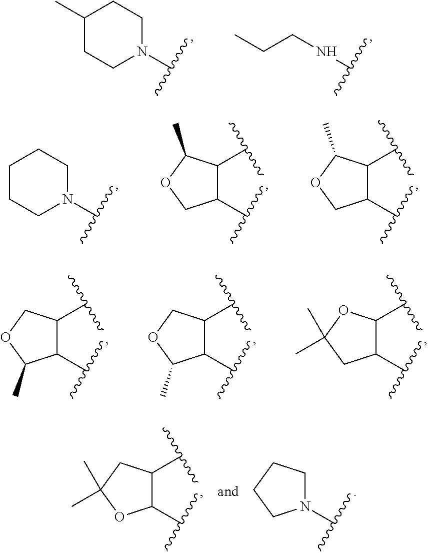

Embodiment 49. The compound of any one of Embodiments 1-48, wherein R² is C₁₋₆ alkyl.

Embodiment 50. The compound of any one of Embodiments 1-48, wherein R² is a 3-7 membered monocyclic carbocyclic ring.

Embodiment 51. The compound of Embodiment 50, wherein R² is a 3-4 membered monocyclic carbocyclic ring.

Embodiment 52. The compound of any one of Embodiments 1-48, wherein R² is a 3-7 membered monocyclic heterocyclic ring having 1-2 heteroatoms independently selected from nitrogen, oxygen, and sulfur.

Embodiment 53. The compound of Embodiment 52, wherein R² is a 4-5 membered monocyclic heterocyclic ring having 1 nitrogen atom.

Embodiment 54. The compound of any one of Embodiments 1-48, wherein R² is L¹-R⁴.

Embodiment 55. The compound of Embodiment 54, wherein L¹ is a C₁₋₂ bivalent saturated straight or branched hydrocarbon chain wherein one methylene unit of the chain is optionally and independently replaced by —C(R)₂—, —N(R)—, —O—, —C(O)—, —OC(O)—, —C(O)O—, —S—, —S(O)—, or —S(O)₂—.

Embodiment 56. The compound of Embodiment 54, wherein R⁴ is a 3-6 membered saturated or partially unsaturated monocyclic carbocyclic ring.

Embodiment 57. The compound of Embodiment 54, wherein R⁴ is a 3-7 membered saturated or partially unsaturated monocyclic heterocyclic ring having 1-2 heteroatoms independently selected from nitrogen, oxygen, and sulfur.

Embodiment 58. The compound of any one of Embodiments 1-57, wherein R⁶ represents independently for each occurrence oxo, halogen, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, or —N(R)CN.

Embodiment 59. The compound of any one of Embodiments 1-58, wherein R⁶ represents independently for each occurrence halogen, —CN, —OR, or —S(O)₂R.

Embodiment 60. The compound of any one of Embodiments 1-59, wherein R⁶ represents independently for each occurrence fluoro, —CN, or —OH.

Embodiment 61. The compound of any one of Embodiments 1-48, wherein R² is independently selected from:

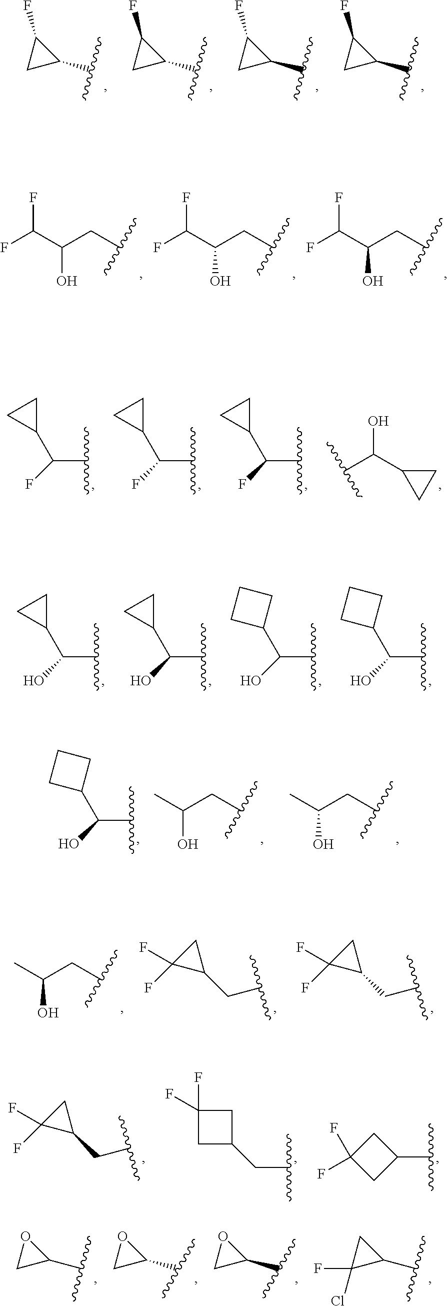

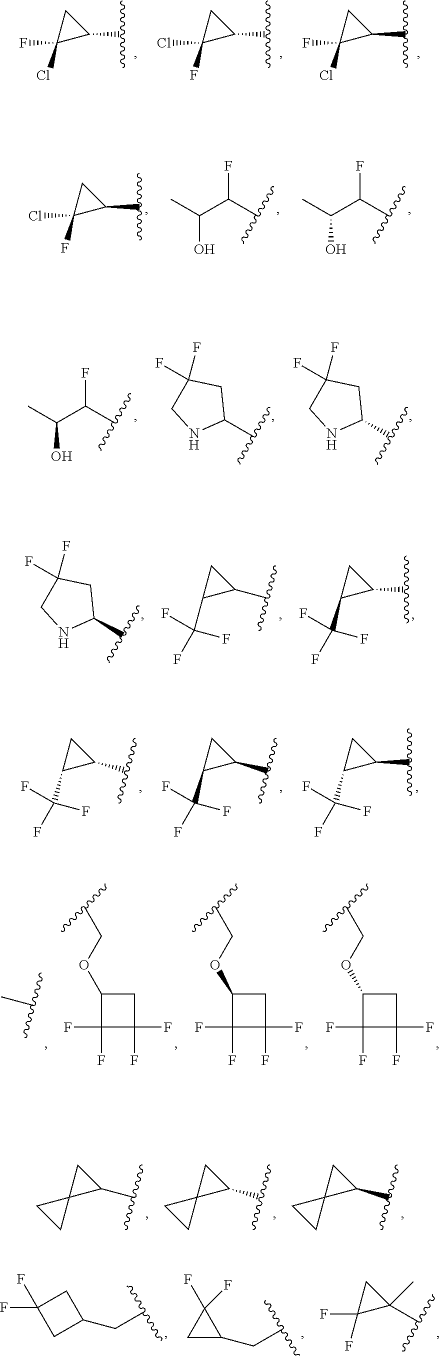

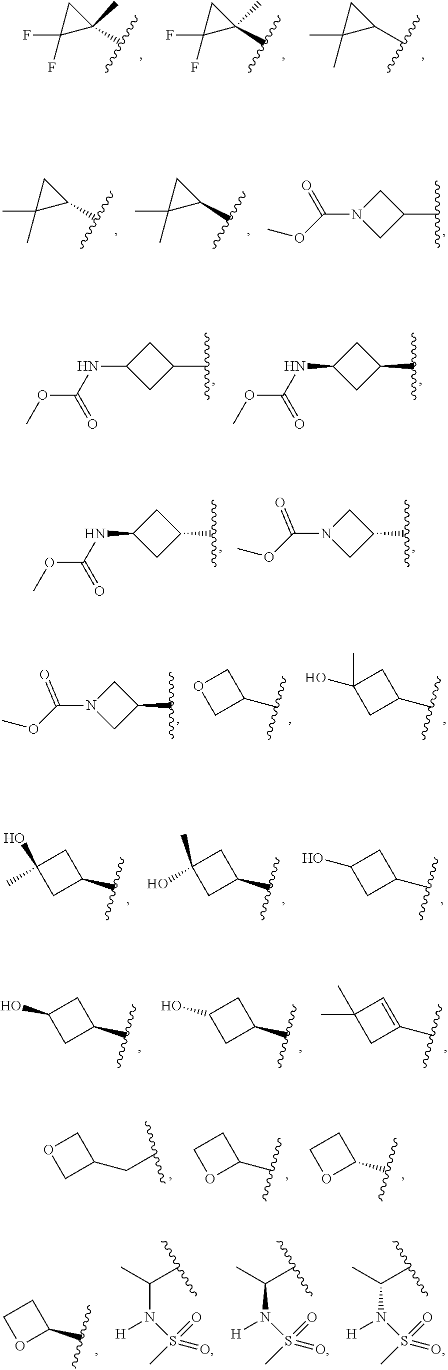

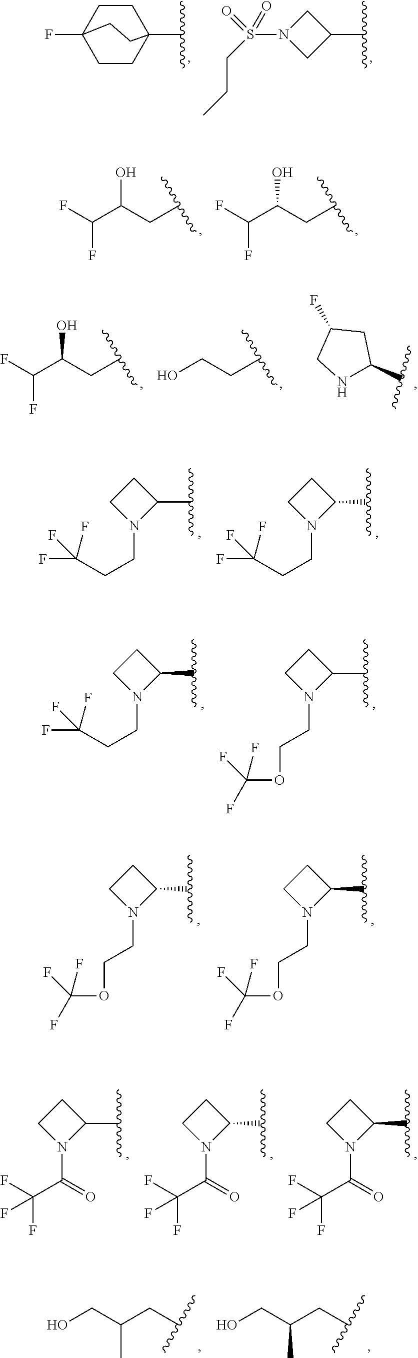

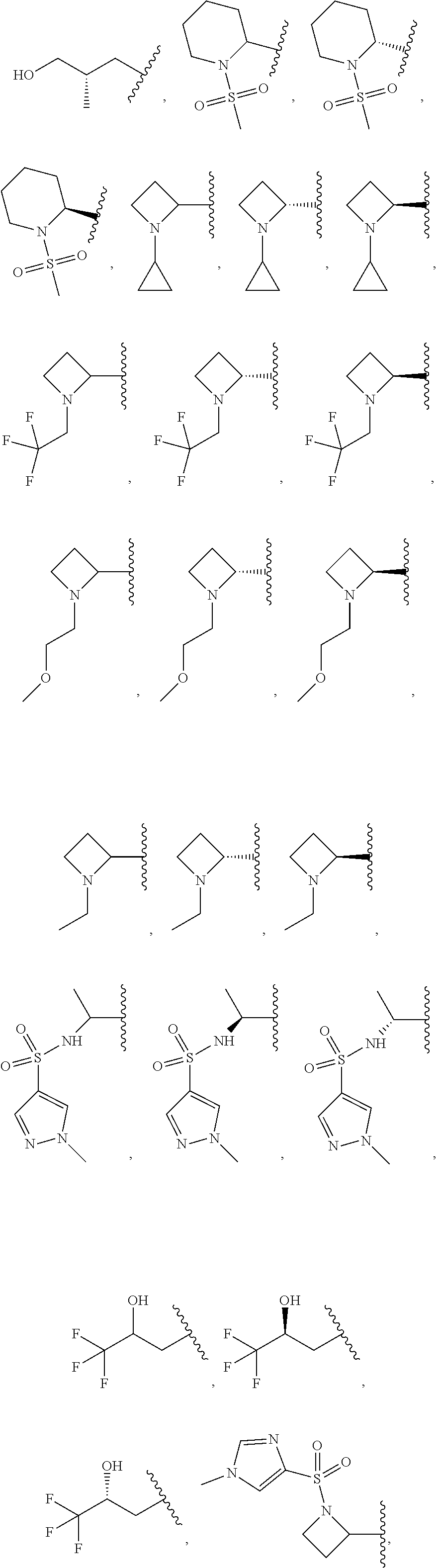

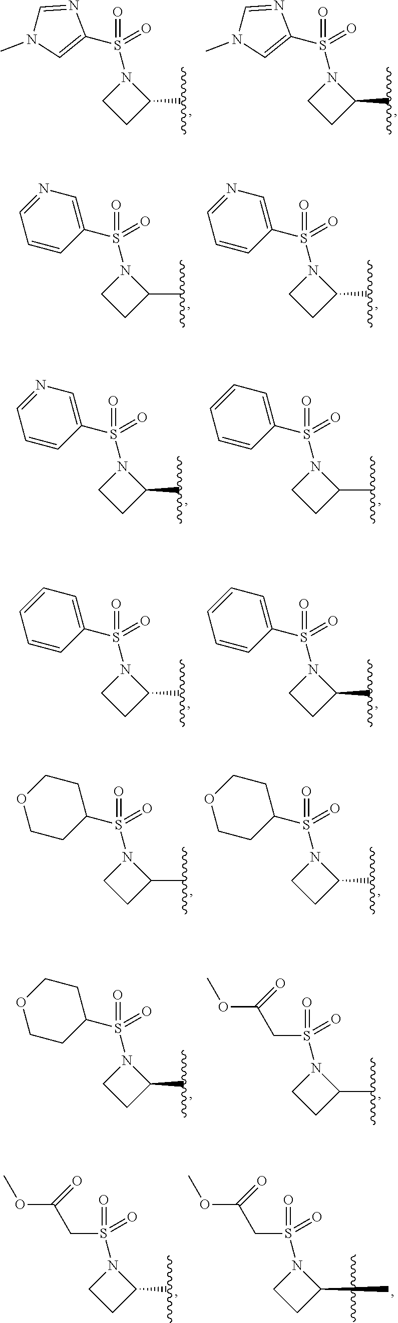

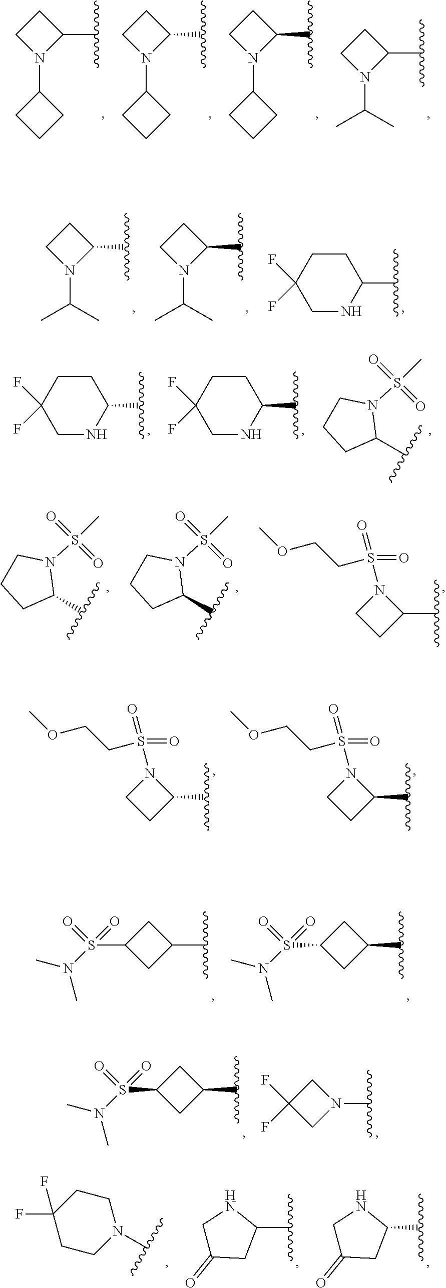

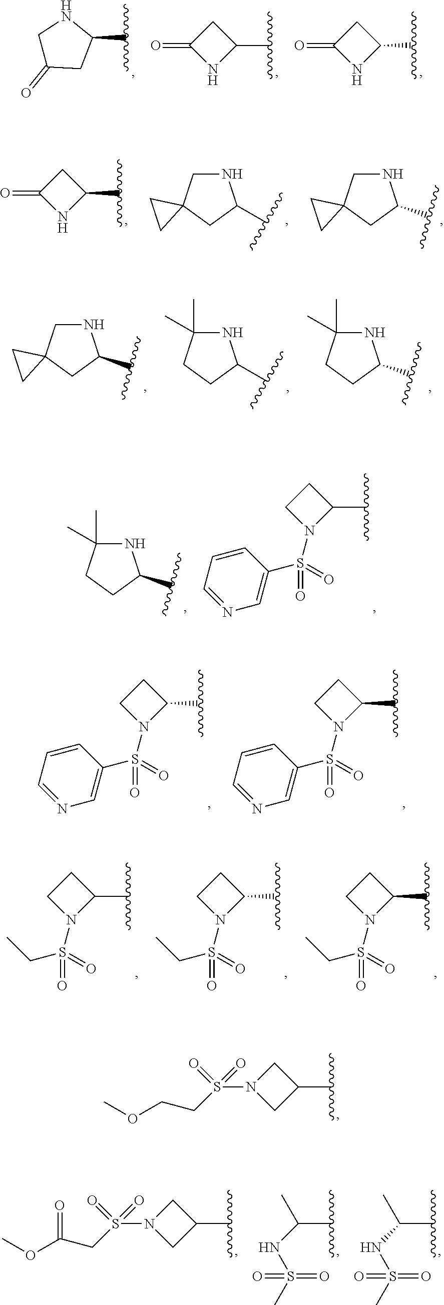

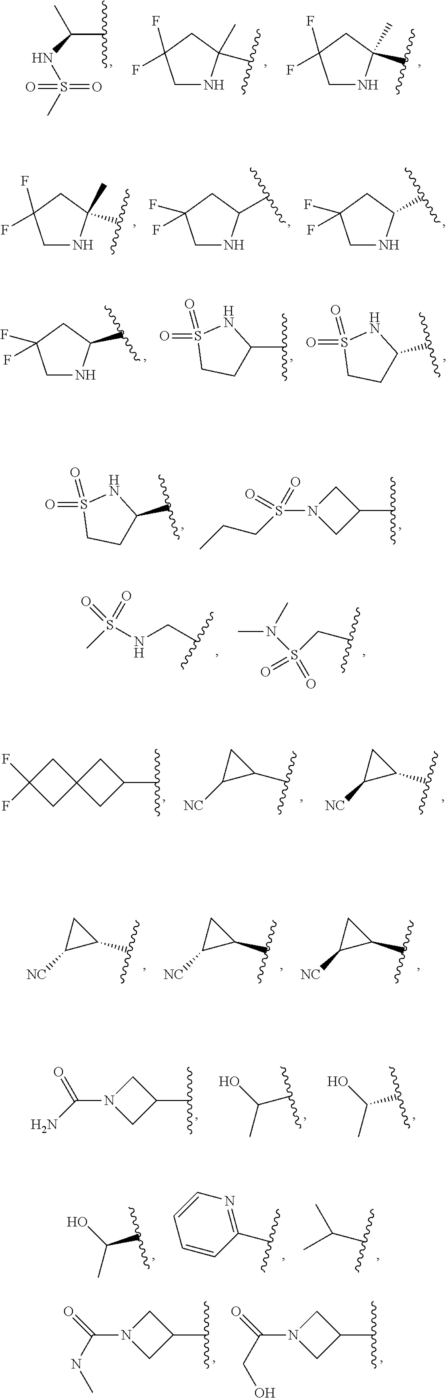

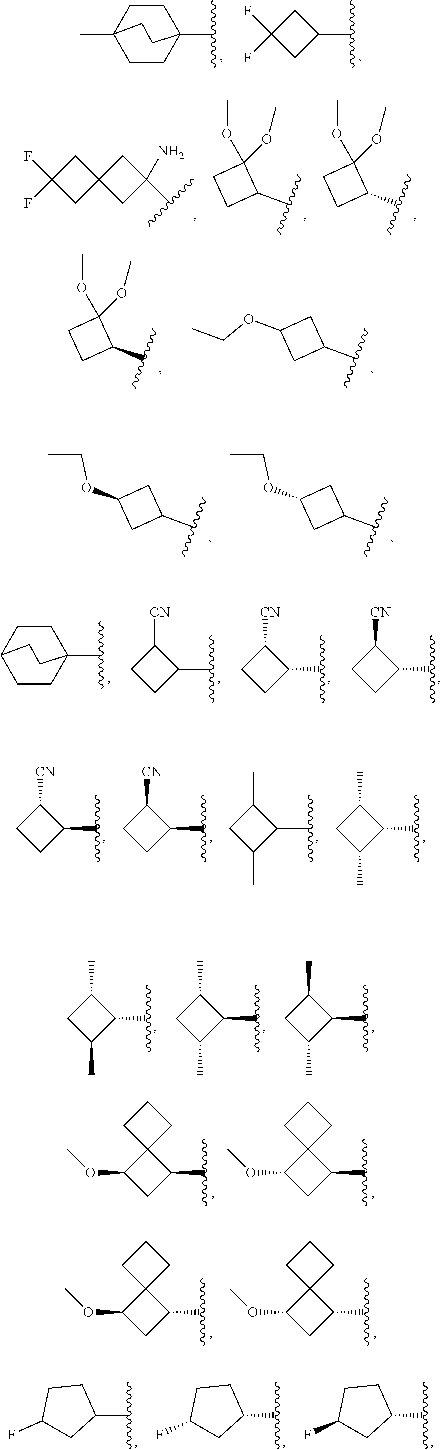

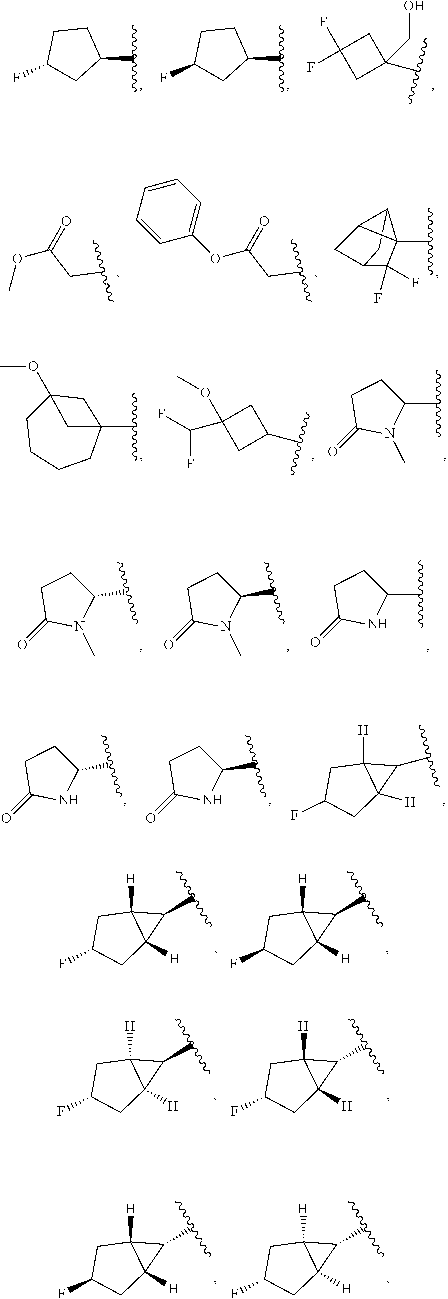

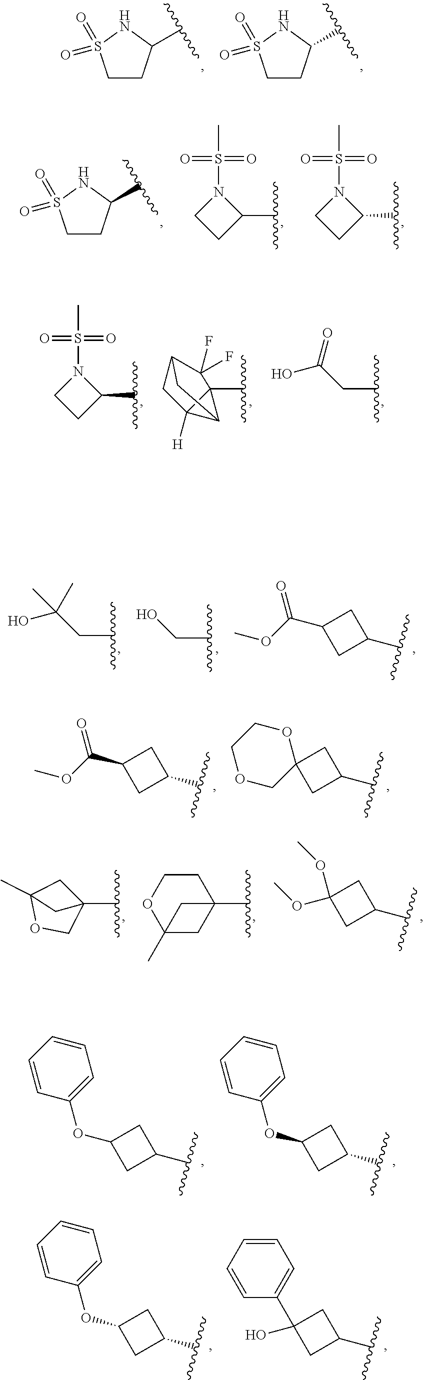

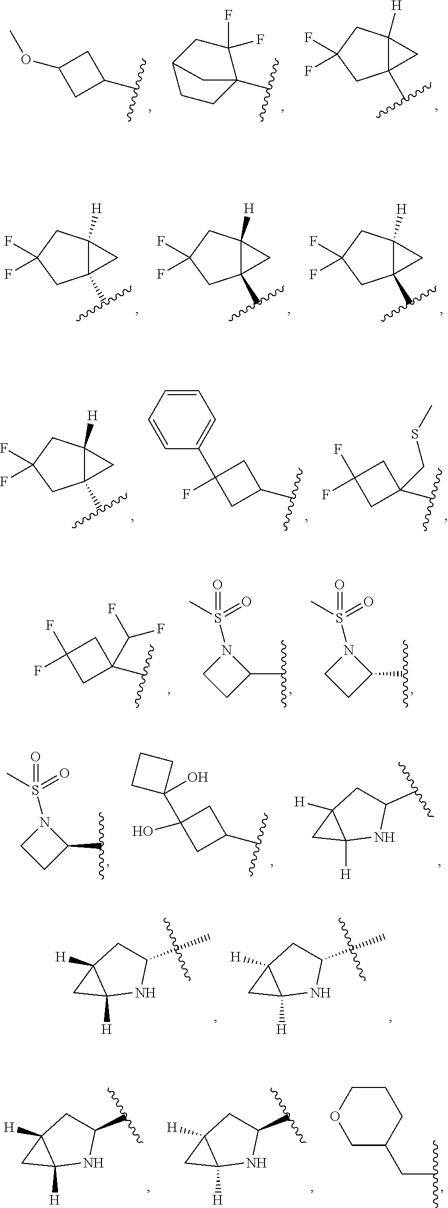

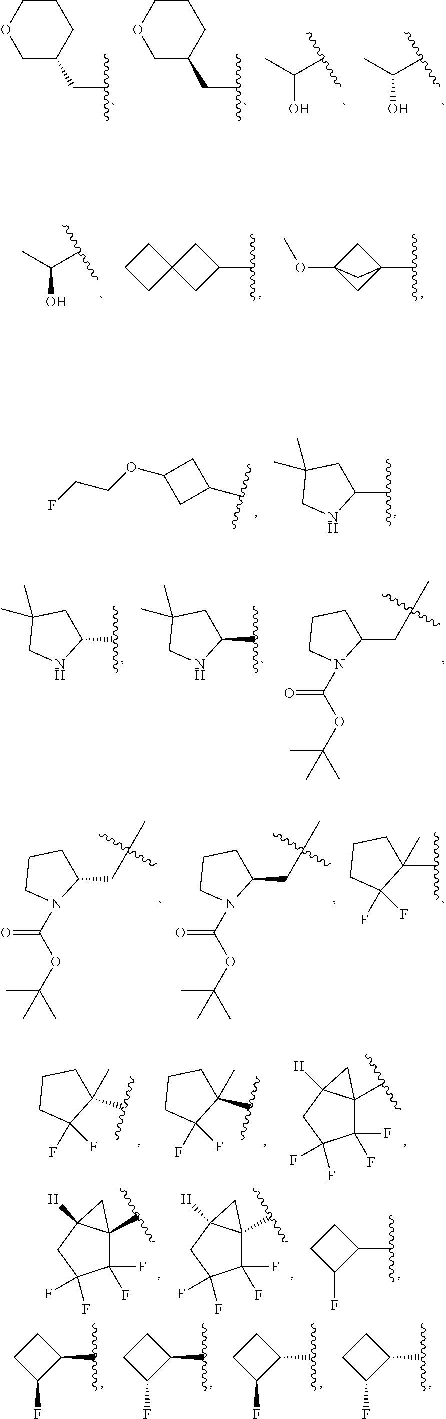

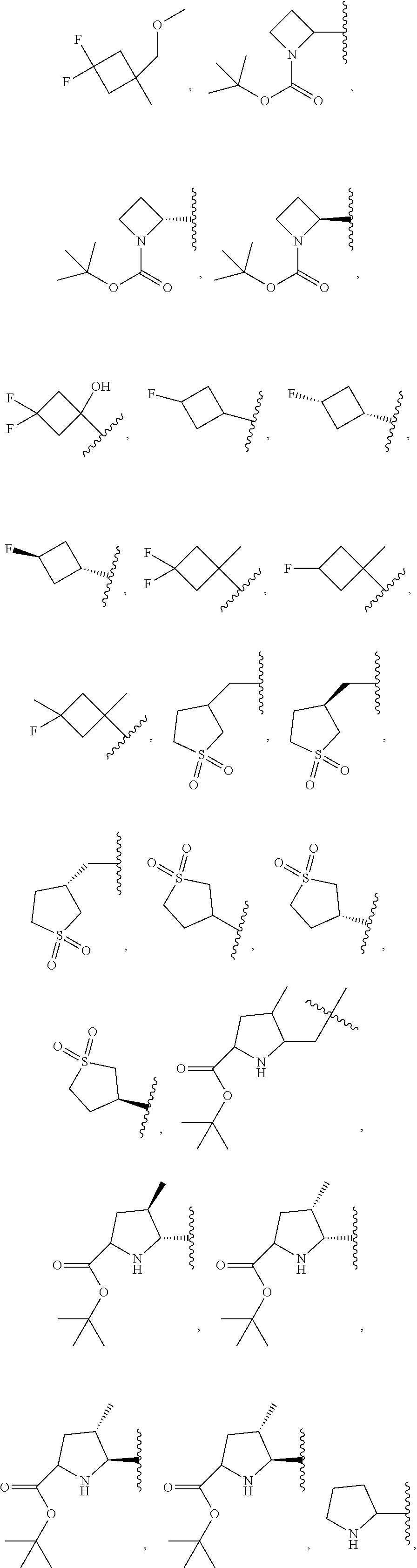

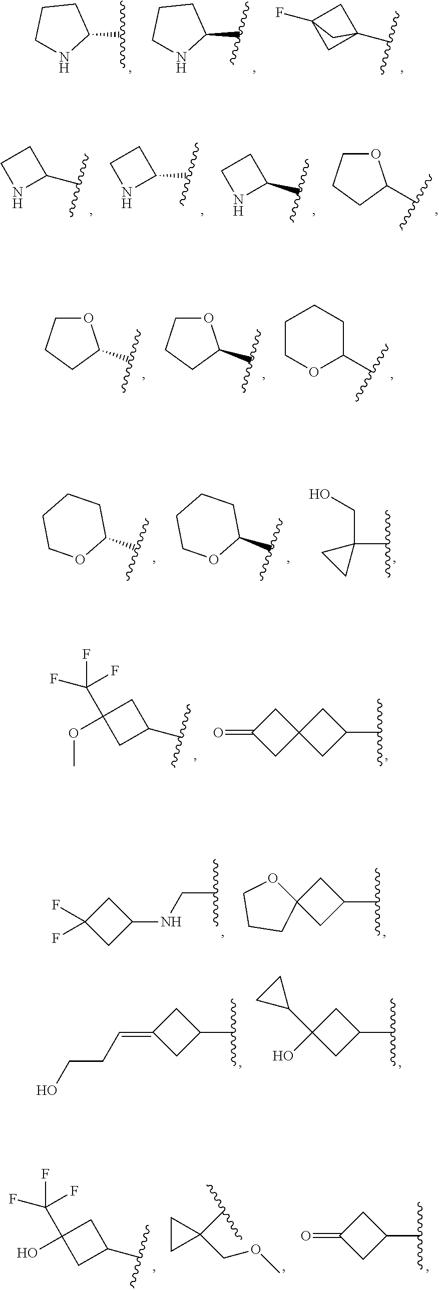

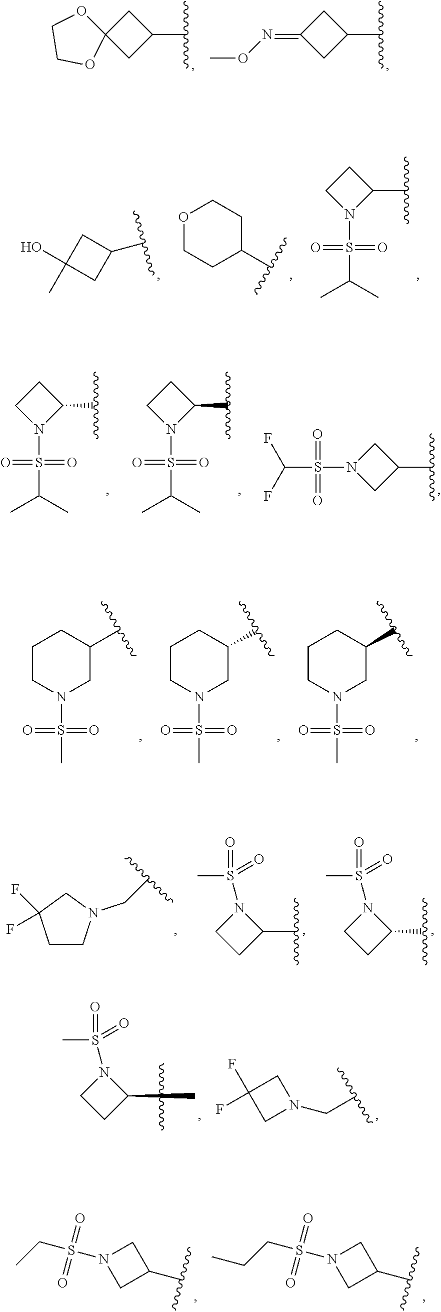

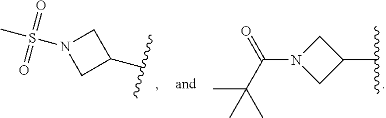

Embodiment 62. The compound of Embodiment 61, wherein R² is selected from: F

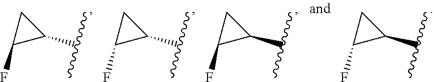

Embodiment 63. The compound of Embodiment 62, wherein R² is

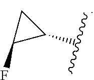 [SMILES: *[C@H]1C[C@@H]1F]

Embodiment 64. The compound of Embodiment 61, wherein R² is selected from:

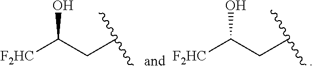 [SMILES: *C[C@H](O)C(F)F ; *C[C@@H](O)C(F)F]

Embodiment 65. The compound of Embodiment 1, wherein the compound is represented by one of the following or a pharmaceutically acceptable salt thereof:

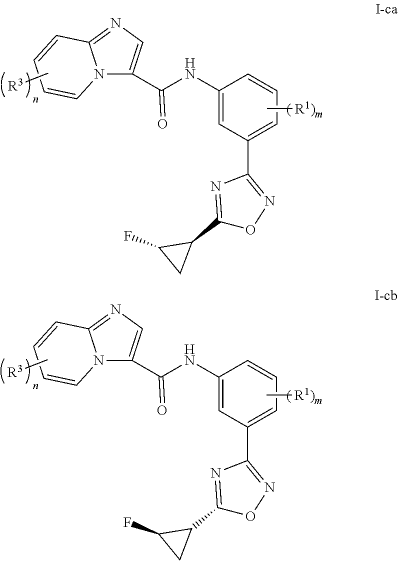

Embodiment 66. The compound of Embodiment 1, wherein the compound is represented by one of the following or a pharmaceutically acceptable salt thereof:

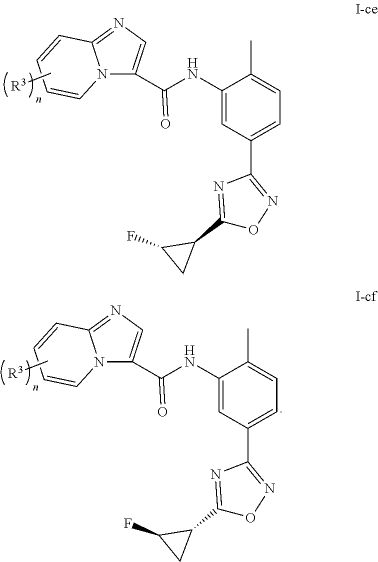

Embodiment 67. The compound of either of Embodiments 65 or 66, wherein n is 1 or 2, wherein at least one R³ is selected from:

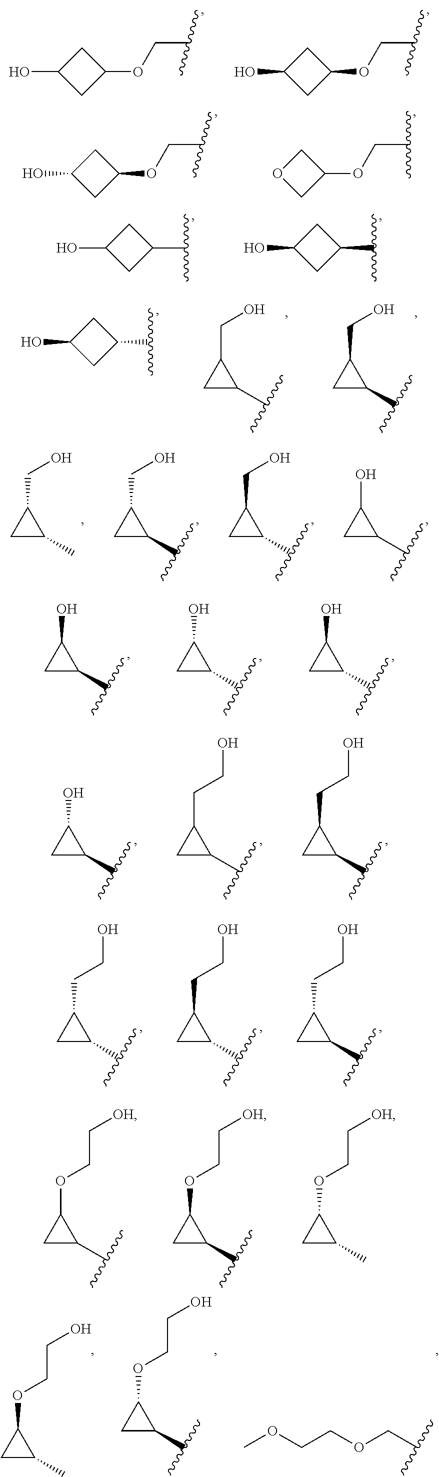

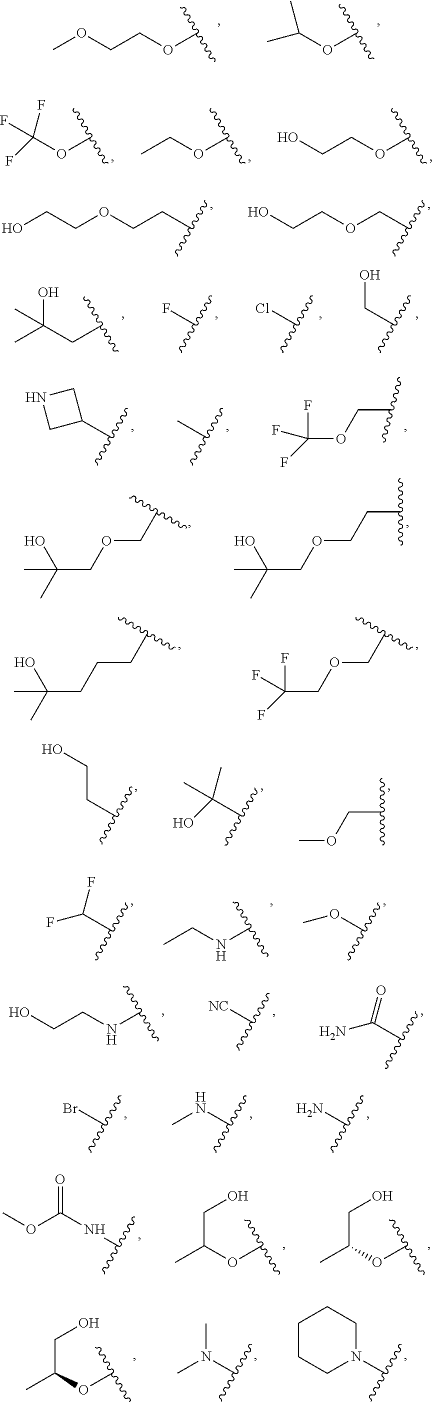

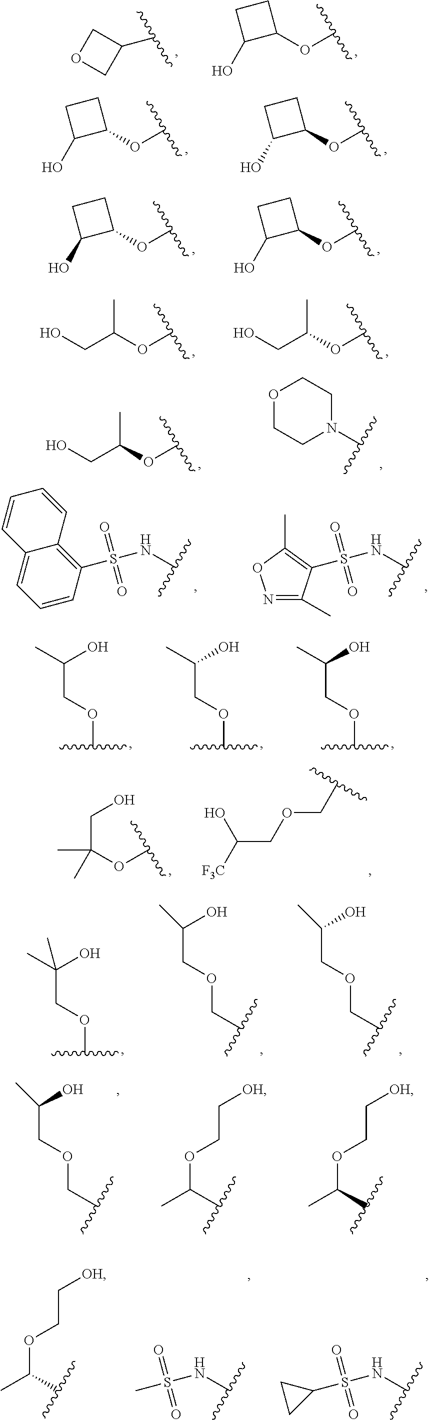

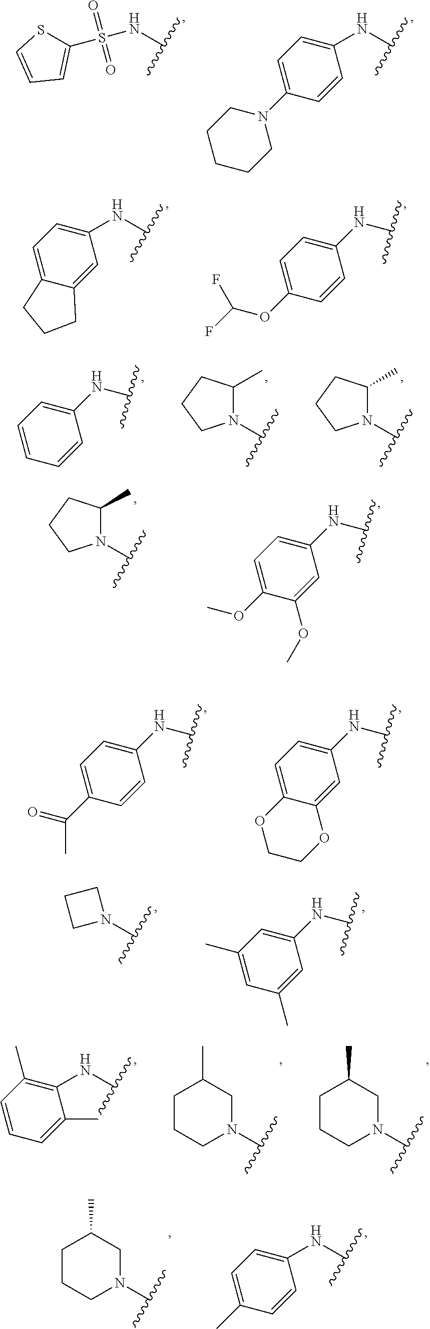

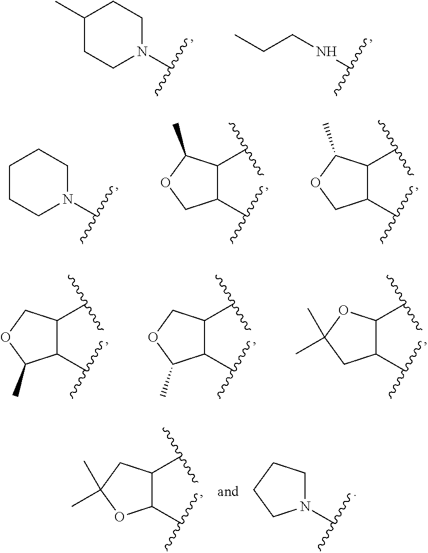

Embodiment 68. The compound of any one of Embodiments 65-67, wherein n is 1.

Embodiment 69. The compound of any one of Embodiments 65-67, wherein n is 2.

Embodiment 70. The compound of Embodiment 1, wherein the compound is represented by one of the following or a pharmaceutically acceptable salt thereof:

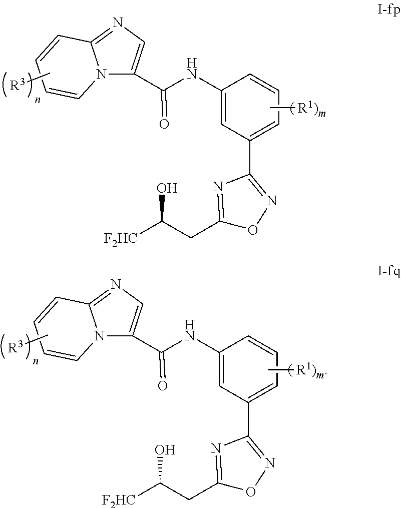

Embodiment 71. The compound of Embodiment 70, wherein the compound is represented by one of the following or a pharmaceutically acceptable salt thereof:

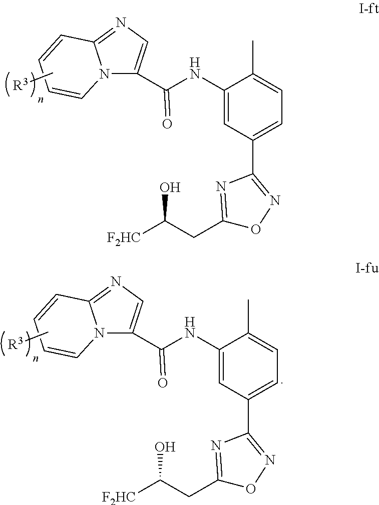

Embodiment 72. The compound of either of Embodiments 71, wherein n is 0.

Embodiment 73. The compound of either of Embodiments 71, wherein n is 1.

Embodiment 74. The compound of Embodiment 73, wherein R³ is selected from:

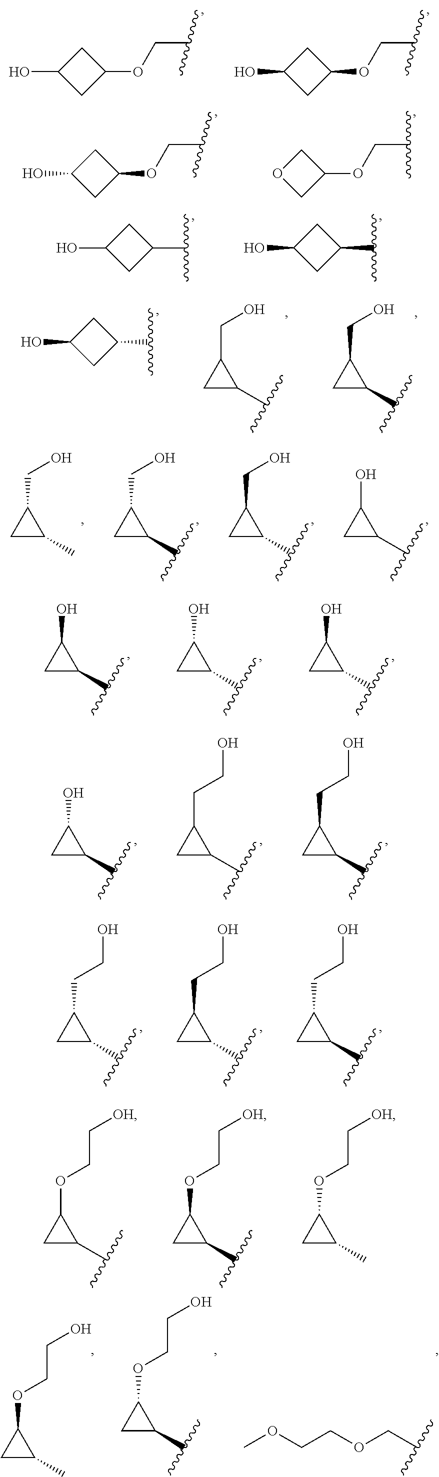

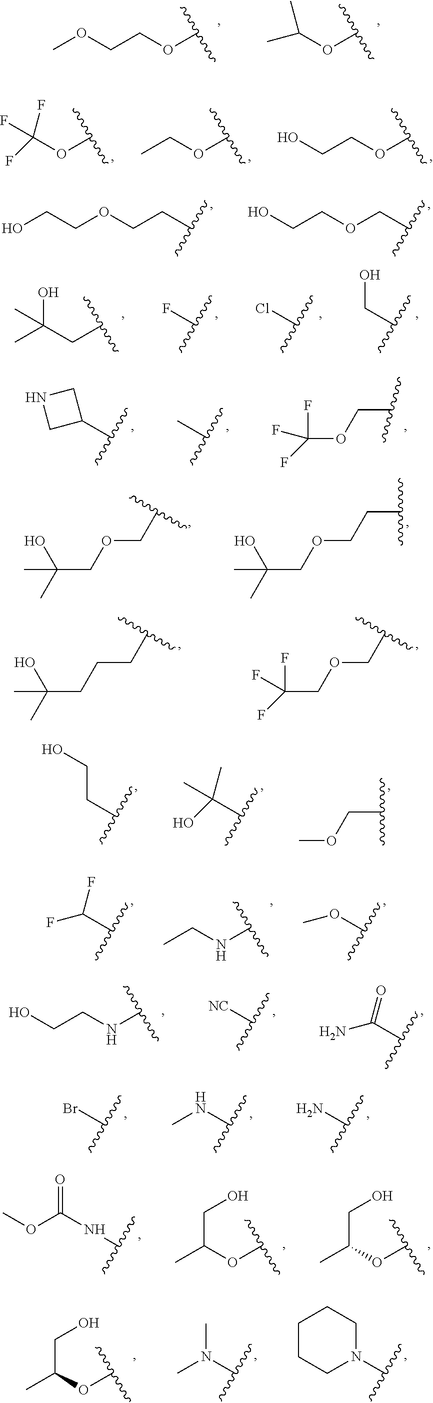

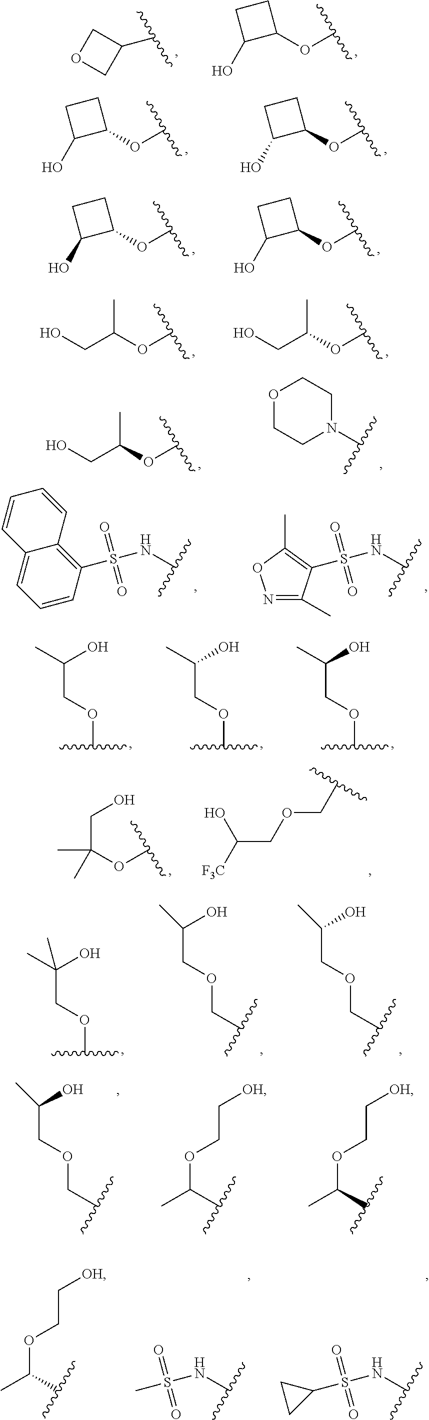

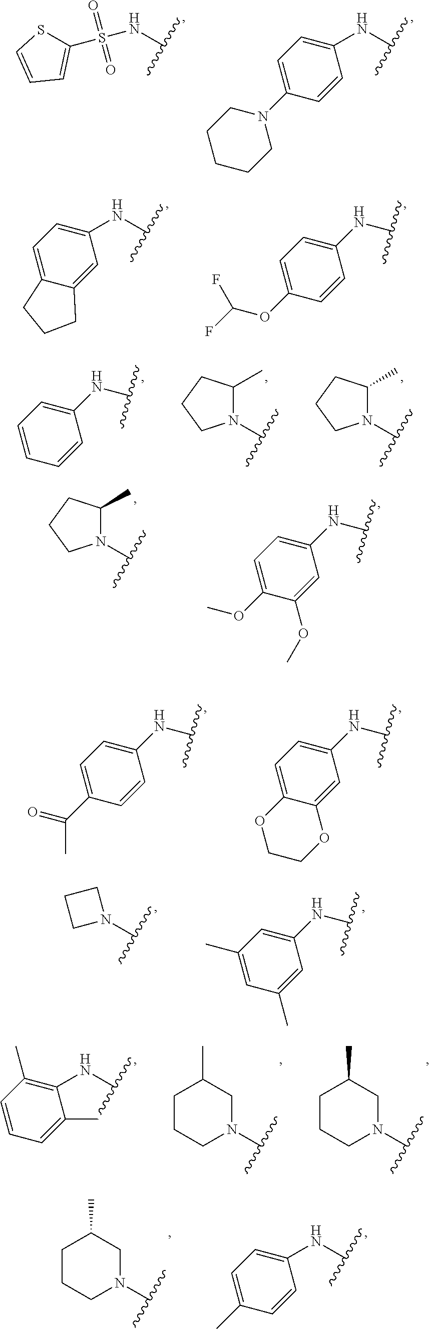

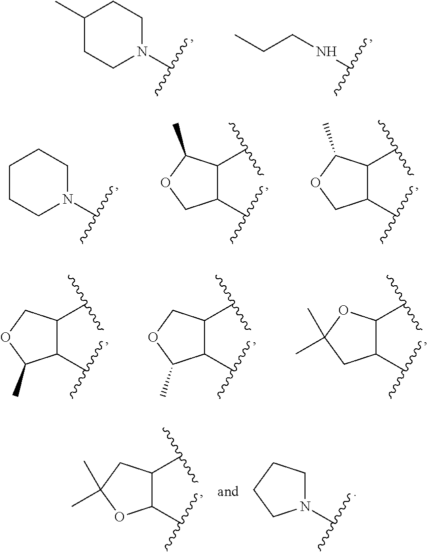

Embodiment 75. The compound of any one of Compounds I-1 to I-446 of Table 1 herein, or a pharmaceutically acceptable salt thereof.

Embodiment 76. A pharmaceutical composition comprising a compound of any one of Embodiments 1-75 and a pharmaceutically acceptable carrier.

Embodiment 77. A method of inhibiting the activity of a c-kit kinase in a patient, comprising administering to said patient a compound of any one of Embodiments 1-75.

Embodiment 78. A method of treating a c-kit kinase mediated disease or disorder in a patient, comprising administering to said patient a compound of any one of Embodiments 1-75.

Embodiment 79. The method according to Embodiment 78, wherein the c-kit kinase mediated disease or disorder is a mast-cell associated disease, a respiratory disease, an inflammatory disorder, an autoimmune disorder, a metabolic disease, a fibrotic disease, a dermatological disease, an allergic disease, a cardiovascular disease, or a neurological disorder.

Embodiment 80. The method according to Embodiment 78, wherein the c-kit kinase mediated disease or disorder is asthma, allergic rhinitis, pulmonary arterial hypertension (PAH), primary pulmonary hypertension (PPH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, food allergy, chronic rhinosinusitis, type I diabetes, type II diabetes, systemic sclerosis, allergic keratoconjunctivitis, vernal keratoconjunctivitis, Crohn's disease, or systemic and cutaneous lupus erythematosus and dermatomyositis.

Embodiment 81. The method according to Embodiment 78, wherein the c-kit kinase mediated disease or disorder is mast cell gastrointestinal disease, prurigo nodularis, allergic conjunctivitis, eosinophilic esophagitis, mast cell activation syndrome, eosinophilic gastritis and/or eosinophilic duodenitis (EG/EoD), ulcerative colitis, eosinophilic gastritis (EG), or eosinophilic colitis (EC).

Embodiment 82. The method of Embodiment 78, wherein the disease or disorder is urticaria.

Embodiment 83. The method of any one of Embodiments 77-82, wherein the patient is a human.

### Examples

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

Abbreviations used in the Examples are described below. Any abbreviations not described are intended to convey their generally accepted meaning.

### Abbreviations

| | |
|---|---|
| Ac | acetyl (C(O)CH₃) |
| aq | aqueous |
| Ar | Aromatic ring |
| AMP | Adenosine monophosphate |
| ATP | Adenosine triphosphate |
| BEH | ethylene bridged hybrid |
| br | Broad |
| BSA | Bovine serum albumin |
| Bz | benzyl (CH₂-phenyl) |
| Boc | tert-butyloxycarbonyl protecting group |
| (Boc)₂O | Di-tert-butyl dicarbonate |
| CDI | 1,1′-Carbonyldiimidazole |
| Conc | Concentration |
| CSF-1R | colony stimulating factor 1 receptor |
| CSH | charged surface hybrid |
| CYP3A4 | Cytochrome P450 3A4 |
| d | doublet |
| Da | Dalton |
| DCM | dichloromethane |
| dioxane | 1,4-dioxane |
| DIPEA | N,N-Diisopropylethylamine |
| DI water | Deionized water |
| DMF | N,N-dimethylformamide |
| DMAP | 4-Dimethylaminopyridine |
| DMSO | dimethyl sulfoxide |
| EDCI | 1-ethyl-3-(3-dimethylaminopropyl)carbodiimide |
| EDTA | Ethylenediamine tetraacetic acid |
| ELISA | Enzyme-linked immunoassay |
| Eq | Molar equivalents |
| (ES⁺) | electrospray ionization, positive mode |
| (ES⁻) | electrospray ionization, negative mode |
| ESI | electrospray ionization |
| Et | ethyl |
| EtOH | ethanol |
| EtOAc | Ethyl acetate |
| FA | Formic acid |
| g | Gram(s) |
| GLP | Good laboratory practice |
| Hal | halogen |
| HATU | 1-[bis(dimethylamino)methylene]-1H-1,2,3-triazolo[4,5- |
| | b]pyridinium 3-oxid hexafluorophosphate |
| HBSS | Hanks' balanced salt solution |
| HEPES | 4-(2-hydroxyethyl)-1-piperazineethanesulfonic acid |
| HPLC | high performance liquid chromatography |
| HRP | Horseradish peroxidase |
| HTRF | Assay Homogeneous Time Resolved Fluorescence Assay |
| h | hour(s) |
| Hz | Hertz |
| H₂ | Hydrogen gas |
| IC₅₀ | 50% inhibitory concentration |
| IPA | Iso-propyl alcohol |
| iPr | iso-propyl |
| J | Coupling constant |
| L | Liter(s) |
| LCMS | liquid chromatography-mass spectrometry |
| LiHMDS | lithium hexamethyldisilazide |
| LLOQ | Lower limit of quantification |
| MDCKII- | Madin-Darby canine kidney II Breast cancer resistance protein |
| BCRP | |
| MDCKII- | Madin-Darby canine kidney II multidrug resistance protein 1 |
| MDR1 | |
| (M + H)⁺ | protonated molecular ion |
| (M − H)⁻ | unprotonated molecular ion |
| M | molar concentration |
| m | multiplet |
| mL | Milliliter(s) |
| mm | Millimiter(s) |
| mmol | Millimole(s) |
| Me | methyl |
| MeOH | Methanol |
| Mn(dpm)₃ | Tris(2,2,6,6-tetramethyl-3,5-heptanedionato)manganese(III) |
| MHz | megahertz |
| min | minute(s) |
| MPLC | Medium pressure liquid chromatography |
| MSD | mass selective detector |
| MW | microwave |
| m/z | mass-to-charge ratio |
| μL | Microliter(s) |
| N₂ | nitrogen gas |
| nL | Nanoliter(s) |
| nm | Nanometer(s) |
| NMP | N-Methyl-2-pyrrolidone |
| NMR | nuclear magnetic resonance (spectroscopy) |
| O₂ | oxygen gas |
| P4HB | poly-4-hydroxybutyrate |
| PBS | Phosphate-buffered saline |
| Pd/C | Palladium on Carbon |
| Pd-170 | XPhos Pd(crotyl)Cl |
| Pd-171 | RuPhos Pd(crotyl)Cl |
| PDA | photodiode array |
| PDGFR | platelet-derived growth factor receptor A |
| Pd(PPh₃)₄ | tetrakis(triphenylphosphine)palladium(0) |
| PMB | 4-methoxybenzyl |
| prep | preparative high performance liquid chromatography |
| HPLC | |
| Ph | phenyl |
| Py | pyridine |
| pos/neg | positive/negative |
| q | quartet |
| QC | Quality control |
| RT | room temperature |
| Rt | retention time |
| RP | reverse phase |
| rpm | Revolutions per minute |
| RuPhos | 2-Dicyclohexylphosphino-2′,6′-diisopropoxybiphenyl |
| s | singlet |
| SM | Starting material |
| sat | saturated |
| SCF | Stem cell factor |
| SCX | solid supported cation exchange (resin) |
| t | triplet |
| tBu | tert-butyl |
| t-BuOH | tert-butanol |
| TFA | Trifluoroacetic acid |
| THF | tetrahydrofuran |
| TEA | triethylamine |
| TLC | Thin layer chromatography |
| Tris | Tris(hydroxymethyl)aminomethane |
| ULOQ | Upper limit of quantification |
| UPLC | ultra performance liquid chromatography |
| UV | ultraviolet |
| v/v | volume/volume |
| VWD | variable wave detector |
| wt | weight |
| wtKIT | Wild type c-kit |
| μm | micrometre |
| μL | microlitre |
| xg | Relative centrifugal force |
| XPhos | 2-Dicyclohexylphosphino-2′,4′,6′-triisopropylbiphenyl |
| ° C. | Degrees Celsius |

Compounds containing one or more stereocenters are a mixture of stereoisomers, unless otherwise stated or described (for example, with use of dashed or wedged bonds denoting stereochemistry). Generally, enhanced stereochemical representation introduces three types of identifiers that can be attached to a stereogenic center. A stereochemical group label is composed from an identifier and a group number. Each stereogenic center marked with wedge bonds belongs to one (and only one) stereochemical group. Grouping allows to specify relative relationships among stereogenic centers.

ABS denotes a stereogenic center where the absolute configuration is known. As used herein, “or” denotes a stereogenic center where the relative configuration is known, but the absolute configuration is not known. The structure represents one stereoisomer that is either the structure as drawn (R, S) or the epimer in which the stereogenic centers have the opposite configuration (S,R). One of skill in the art would understand that if a single stereogenic center is present, the designation “or” represents a single isomer for which the absolute configuration is not known. In some such instances, two compounds may be depicted identically with “or” at the single stereogenic center, see, e.g., compounds I-170 and I-171, one of which has a single stereogenic center which is in the R configuration, the other of which is in the S configuration. The designations “and” and “&” are used interchangeably and denote a mixture of stereoisomers. It can be a pair of enantiomers or all the diastereomers.

All starting materials and solvents were obtained either from commercial sources or prepared according to the literature. Unless otherwise stated all reactions were stirred. Organic solutions were routinely dried over anhydrous magnesium sulfate or other drying agent.

### Example 1—Synthesis of Compounds

The compounds of Table 1 were synthesized by one of the Schemes below.

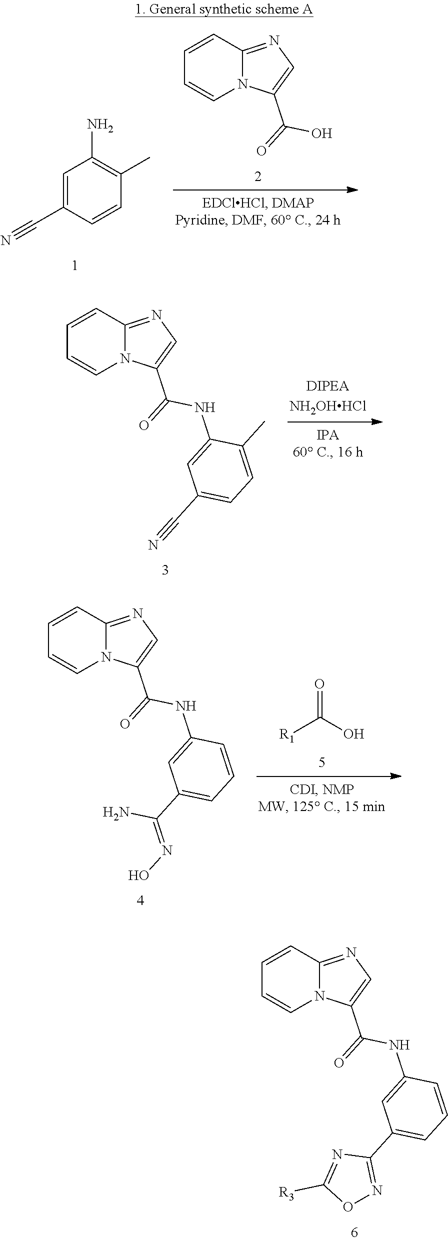

### Synthesis of Intermediate 3:

To a stirred solution of 3-amino-4-methylbenzonitrile 1 (1 eq), imidazo[1,2-a]pyridine-3-carboxylic acid 2 (1.05 eq), DMAP (1.3 eq) in DMF (0.1 M) was added pyridine (3 eq) into the reaction mixture at RT. After 10 min EDC.HCl (also known as EDCI.HCl) (3 eq) was added. The reaction mixture was heated to 60° C. for 16 h. Upon heating, it slowly turned to clear brown solution. The reaction was monitored by TLC and LCMS. If SM was still observed an additional equivalent of EDC.HCl was added to the reaction mixture which was stirred for an additional 8 h at 60° C. The reaction mixture was then poured dropwise to ice water (1.5 L) and stirred for 30 min. The resulting solid was filtered, washed with water (200 mL) and hexanes (500 mL) and dried under reduced pressure to afford intermediate 3 as off-white solid in a greater than 90% crude yield.

### Synthesis of Intermediate 4:

To a stirred solution of carboxamide 3 (1 eq) in IPA (0.35 M) was added DIPEA (2 eq) and hydroxylamine hydrochloride (2 eq) sequentially at RT. The reaction mixture was then heated to 60° C. for 16 h. Progress of the reaction was monitored by TLC and LCMS. After completion of the reaction, the reaction mixture was cooled to RT. The resulting solids were collected by vacuum filtration, washed with 50% IPA & water, dried and triturated in EtOAc at 60° C. for 4 h. The obtained solid was filtered and dried under vacuum to afford (Z)—N-(5-(N′-hydroxycarbamimidoyl)-2-methylphenyl)imidazo[1,2-a]pyridine-3-carboxamide or common intermediate 4 in a 76% yield as an off-white solid.

### Synthesis of Intermediate 6:

To stirred solution of carboxylic acid 5 (1 eq) in NMP (0.03 M) was added 1′-carbonyldiimidazole (1 eq) at RT and stirred. After 20 min, intermediate 4 (0.5 eq) was added and the reaction mixture which was stirred for 30 min at RT. The reaction mixture was subjected to microwave irradiation at 125° C. in a microwave reactor for 15-20 min. Progress of the reaction was monitored by TLC and LCMS. The reaction mixture was poured into ice water. The precipitate obtained was filtered and dried under reduced pressure to get the crude product. The obtained crude material was purified by prep-HPLC purification, to give desired final compound 6 in a 20-80% yield.

### Alternative Oxadiazole Cyclization:

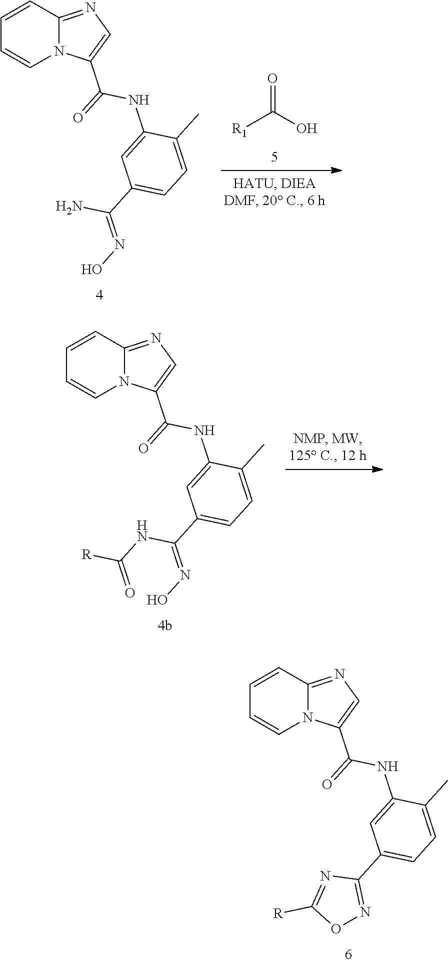

### Synthesis of Intermediate 4b:

To a solution of carboxylic acid 5 (1.5 eq) in DMF (0.1M) was added oxime intermediate 4 (1 eq) and DIEA (2 eq) and stirred at 20° C. for 10 min. HATU (1.5 eq) was then added to the reaction mixture and stirred at 20° C. for 6 h. LCMS showed intermediate 4 was consumed completely and desired mass was detected. The mixture was diluted with EA (1 L). Then the mixture washed with water (1 L) and saturated brine (1 L). The organic layer was dried over anhydrous Na₂SO₄. The residue was used in next step directly without further purification. Compound 4b was obtained as brown oil in 99% crude yield.

### Synthesis of Intermediate 6:

A solution of intermediate 4b (1 eq) in NMP (0.1 M) was stirred at 120° C. and subjected to microwave irradiation for 12 h. The reaction mixture was monitored by TLC and LCMS. The mixture was quenched with 500 mL of ice water and extracted with 500 mL of EtOAc three times. The combined organic phase was washed with 100 mL of brine, dried over anhydrous Na₂SO₄, filtered and concentrated to give a colored residue. The residue was purified by column chromatography (SiO₂, PE:EtOAc, 0:1 to 1:2). The residue of the desired fractions was triturated with EA (50 mL) for 5 min. Final compound 6 was obtained, typically as solid, in 20-60% yield.

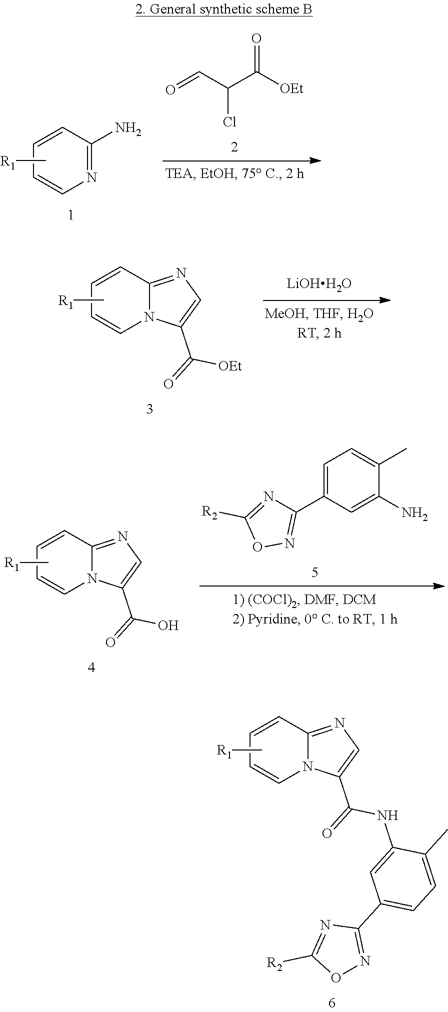

### Synthesis of Intermediate 3.

To a solution of 1 (1 eq) and chloroaldehyde 2 (1.2 eq) in EtOH (200 mL, 0.4 M) was added TEA (1.1 eq). The mixture was stirred at 75° C. for 2 h. Reaction was monitored via TLC and LCMS. Upon consumption of 1, the mixture was concentrated in vacuo. The residue was purified by column chromatography (SiO₂, PE:EA, 3:2 to 1:1). The substituted imidazopyridine 3 was obtained as brown oil with a 20-65% yield.

### Synthesis of Intermediate 4.

To a solution of 3 (1 eq) in THE (80 mL) and MeOH (10 mL) and water (10 mL) was added LiOH·H₂O (3 eq), The mixture was stirred at 25° C. for 2 h. LCMS showed 3 was consumed completely and one main peak with desired mass was detected. The reaction mixture was diluted with water (50 mL) and extracted with DCM:TPA in a ratio of 10:1 (200 mL×3). The combined organic layers were dried over Na₂SO₄, filtered and concentrated under reduced pressure to give a residue. Compound 4 was obtained as a crude material, without further purification as a yellow solid.

### Synthesis of Final Compound 6.

To a stirred solution of the substituted pyrazolo[1,5-a]pyridine-3-carboxylic acid 4 (1 eq) in DCM (1 M) at 0° C., oxalyl chloride (5 eq) was added dropwise and followed by catalytic DMF and reaction mixture was stirred at RT for 1 h. After completion of reaction, the reaction mixture was concentrated under vacuum. The obtained crude material was dissolved in DCM (1 mL) and pyridine (5.0 mL) and aniline 5 (1 eq) was added at 0° C. and then the reaction mixture was stirred at RT for 2 h. Progress of the reaction was monitored by TLC. After completion of the reaction, the mixture was diluted with water and extracted with EtOAc. The organic layer was dried over Na₂SO₄ and concentrated under vacuum to get the crude material. The obtained crude material was purified by column chromatography (SiO₂, hexanes:EtOAc, 1:1 to 1:3). The obtained solid was triturated with methanol to afford the final compound 7 with a 20-65% yield as an off-white solid.

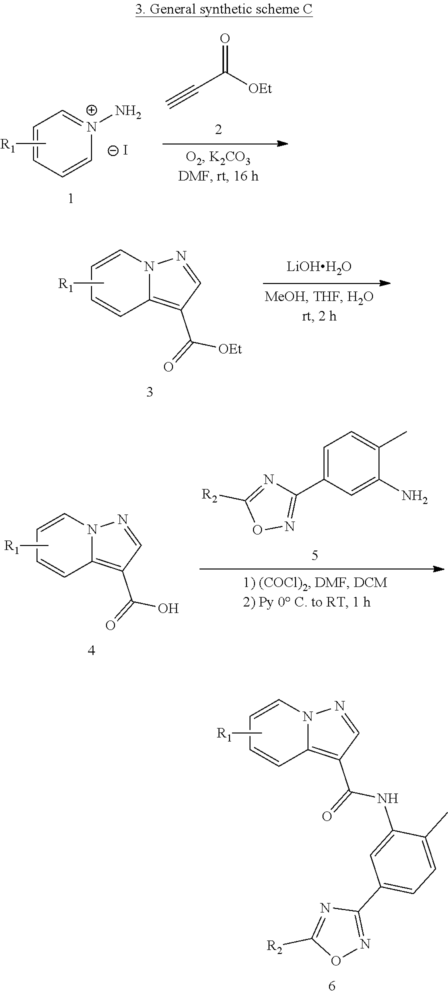

### Synthesis of Intermediate 3:

To a stirred solution of the aminopyridinium 1 (1.0 eq) in DMF (0.2 M) was added ethyl propionate 2 (1.1 eq) and K₂CO₃ (1.5 eq) and reaction mixture stirred under an atmosphere of O₂, at RT for 16 h. Progress of the reaction was monitored by TLC and LCMS. After completion of the reaction, reaction mixture was diluted using EtOAc (100 mL) and washed with water (50 mL×3). The combined organic layers were concentrated on vacuum to obtain the crude material. The crude material was purified by column chromatography (SiO₂, hexane:EtOAc, 1:9) to afford the substituted ethyl pyrazolo[1,5-a]pyridine-3-carboxylate 3 in a 30-75% yield as an off white solid.

### Synthesis of Intermediate 4:

To a stirred suspension of substituted ethyl pyrazolo[1,5-a] pyridine-3-carboxylate 3 (1 eq) in THF:MeOH:H₂O (4:2:1 ratio, 0.3 M) was added LiOH·H₂O (4 eq) at 10° C. and then the reaction mixture was stirred at the RT for 2 h. Progress of the reaction was monitored by TLC and HPLC. After completion of the reaction, the reaction mixture was concentrated under vacuum to obtain the crude material. The crude material was diluted with water and washed with diethyl ether. The aq. layer was acidified with 1N HCl (pH=4-5), and the desired material precipitated out. The obtained solid was filtered and dried to afford the substituted pyrazolo[1,5-a]pyridine-3-carboxylic acid 4 with a 50-80% yield as an off-white solid.

### Synthesis of Intermediate 6:

To a stirred solution of the substituted pyrazolo[1,5-a]pyridine-3-carboxylic acid 4 (1 eq) in DCM (1 M) at 0° C., oxalyl chloride (5 eq) was added dropwise and followed by catalytic DMF and reaction mixture was stirred at RT for 1 h. After completion of reaction, reaction mixture was concentrated under vacuum. The obtained crude material was dissolved in DCM (1 mL) and pyridine (5.0 mL) and aniline 5 (1 eq) were added at 0° C. and then the reaction mixture was stirred at RT for 2 h. Progress of the reaction was monitored by TLC. After completion of the reaction, the mixture was diluted with water and extracted with EtOAc. The organic layer was dried over Na₂SO₄ and concentrated under vacuum to get the crude material. The obtained crude material was purified by column chromatography (SiO₂, hexane:EtOAc, 1:3). The obtained solid was triturated with methanol to afford the final compound with a 20-65% yield as an off-white solid.

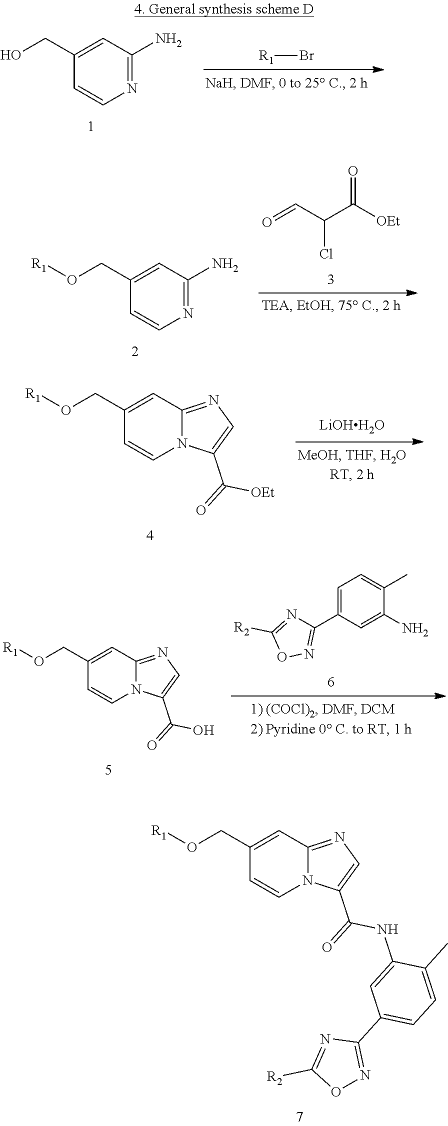

### Synthesis of Intermediate 2.

To a solution of (2-aminopyridin-4-yl) methanol 1 (1 eq) in DMF (0.8 M) was added NaH (2 eq, 60% dispersion in mineral oil) at 0° C. and stirred for 0.5 h. Then the corresponding alkyl halide (2 eq) was added and stirred at 25° C. for 1.5 h. Reaction was monitored via TLC and LCMS. Upon consumption of 1, reaction mixture was quenched with water (500 mL), then extracted with EtOAc (500 mL) and washed with water (500 mL×3). The combined organic layers were dried over Na₂SO₄, filtered and concentrated under reduced pressure to give a crude residue that was used without further purification.

### Synthesis of Intermediate 4.

To a solution of 2 (1 eq) and chloroaldehyde 3 (1.2 eq) in EtOH (200 mL, 0.4 M) was added TEA (1.1 eq). The mixture was stirred at 75° C. for 2 h. Reaction was monitored via TLC and LCMS. Upon consumption of 2 the mixture was concentrated in vacuo. The residue was purified by column chromatography (SiO₂, PE: EtOAc, 3:2 to 1:1). Alkyl ether 4 was obtained as brown oil with a 30-42% yield.

### Synthesis of Intermediate 5.

To a solution of 4 (1 eq) in THE (80 mL) and MeOH (10 mL) and water (10 mL) was added LiOH·H₂O (3 eq), The mixture was stirred at 25° C. for 2 h. LCMS showed 4 was consumed completely and one main peak with desired mass was detected. The reaction mixture was diluted with water (50 mL) and extracted with DCM:IPA in a ratio of 10:1 (200 mL×3). The combined organic layers were dried over Na₂SO₄, filtered and concentrated under reduced pressure to give a residue. 5 was obtained as a crude material, without further purification as a yellow solid.

### Synthesis of Final Compound 7.

To a stirred solution of the substituted imidazo[1,2-a]pyridine-3-carboxylic acid 5 (1 eq) in DCM (1 M) at 0° C., oxalyl chloride (5 eq) was added dropwise and followed by catalytic DMF and reaction mixture was stirred at RT for 1 h. After completion of reaction, reaction mixture was concentrated under vacuum. The obtained crude material was dissolved in DCM (1 mL) and pyridine (5.0 mL) and aniline 6 (1 eq) was added at 0° C. and then the reaction mixture stirred RT for 1 h. Progress of the reaction was monitored by TLC. After completion of the reaction, the mixture was diluted with water and extracted with EtOAc. The organic layer was dried over Na₂SO₄ and concentrated under vacuum to get the crude material. The obtained crude material was purified by column chromatography (SiO₂, hexane: EtOAc, 1:1 to 1:3). The obtained solid was triturated with methanol to afford the final compound 7 with a 20-65% yield as an off-white solid.

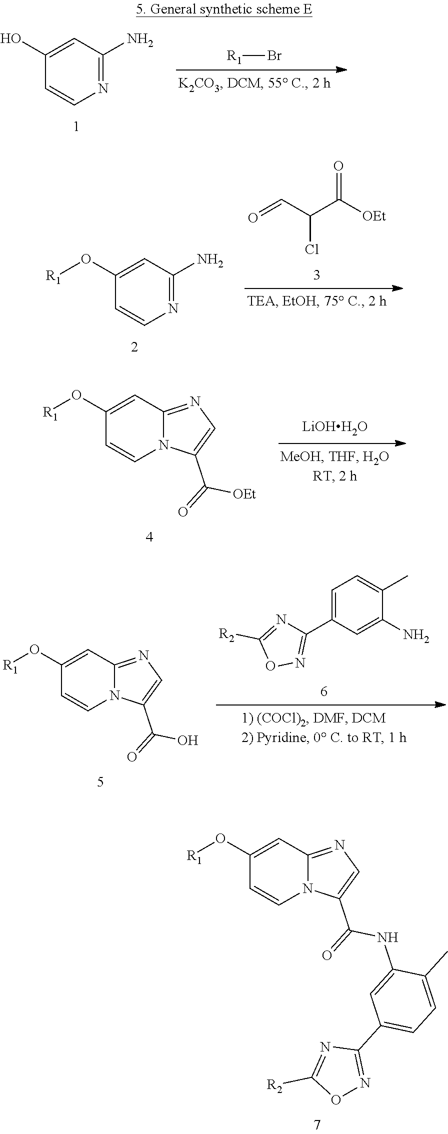

### Synthesis of Intermediate 2.

To a solution of 1 (1 eq) in DCM (0.8 M) was added potassium carbonate (3 eq) at 0° C. and stirred for 0.5 h. Then the corresponding alkyl halide (2 eq) was added and stirred at 55° C. for 1.5 h. Reaction was monitored via TLC and LCMS. Upon consumption of 1, reaction mixture was quenched with water (500 mL), then extracted with EtOAc (500 mL) and washed with water (500 mL×3). The combined organic layers were dried over Na₂SO₄, filtered and concentrated under reduced pressure to give a crude residue that was used without further purification.

### Synthesis of Intermediate 4.

To a solution of 2 (1 eq) and chloroaldehyde 3 (1.2 eq) in EtOH (200 mL, 0.4 M) was added TEA (1.1 eq). The mixture was stirred at 75° C. for 2 h. Reaction was monitored via TLC and LCMS. Upon consumption of 2, the mixture was concentrated in vacuo. The residue was purified by column chromatography (SiO₂, PE:EtOAc, 3:2 to 1:1). Alkyl ether 4 was obtained as brown oil with a 40-65% yield.

### Synthesis of Intermediate 5.

To a solution of 4 (1 eq) in THE (80 mL) and MeOH (10 mL) and H₂O (10 mL) was added LiOH·H₂O (3 eq), The mixture was stirred at 25° C. for 2 h. LCMS showed 4 was consumed completely and one main peak with desired mass was detected. The reaction mixture was diluted with water (50 mL) and extracted with DCM:TPA in a ratio of 10:1 (200 mL×3). The combined organic layers were dried over Na₂SO₄, filtered and concentrated under reduced pressure to give a residue. 5 was obtained as a crude material, without further purification as a yellow solid. Quantitative yield is assumed.

### Synthesis of Final Compound 7.

To a stirred solution of the substituted imidazo[1,2-a]pyridine-3-carboxylic acid 5 (1 eq) in DCM (1 M) at 0° C., oxalyl chloride (5 eq) was added dropwise and followed by catalytic DMF and reaction mixture was stirred at RT for 1 h. After completion of reaction, reaction mixture was concentrated under vacuum. The obtained crude material was dissolved in DCM (1 mL) and pyridine (5.0 mL) and aniline 6 (1 eq) was added at 0° C. and then the reaction mixture stirred RT for 1 h. Progress of the reaction was monitored by TLC. After completion of the reaction, the mixture was diluted with water and extracted with EtOAc. The organic layer was dried over Na₂SO₄ and concentrated under vacuum to get the crude material. The obtained crude material was purified by (SiO₂, hexane:EtOAc, 1:3). The obtained solid was triturated with methanol to afford the final compound 7 with a 20-65% yield as an off-white solid.

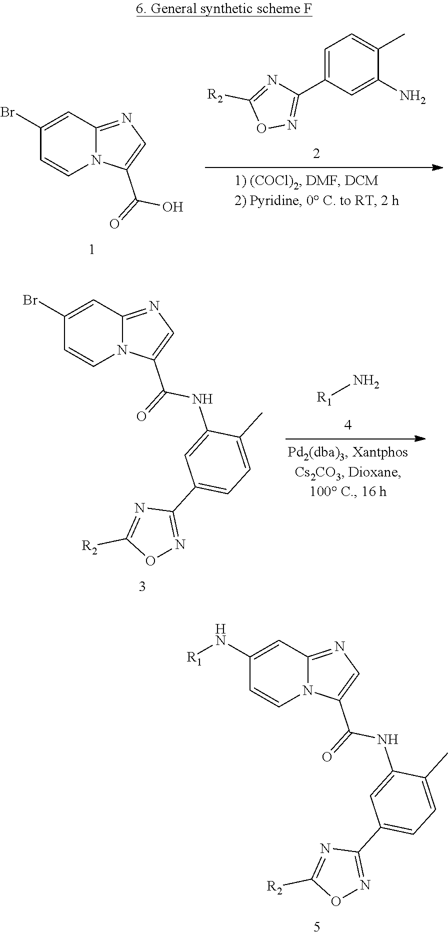

### Synthesis of Intermediate 3.

To a stirred solution of carboxylic acid 1 (1 eq) in DCM (1 M) at 0° C., oxalyl chloride (5 eq) was added dropwise and followed by catalytic DMF and reaction mixture was stirred at RT for 1 h. After completion of reaction, reaction mixture was concentrated under vacuum. The obtained crude material was dissolved in DCM (1 mL) and pyridine (0.2 M) and aniline 2 (1 eq) was added at 0° C. and then the reaction mixture stirred at RT for 2 h. Progress of the reaction was monitored by TLC. After completion of the reaction, the mixture was diluted with water and extracted with EtOAc. The organic layer was dried over Na₂SO₄ and concentrated under vacuum to get the crude material. The obtained crude material was purified by column chromatography (SiO₂, hexane:EtOAc, 1:3). The obtained solid was triturated with methanol to afford the common intermediate 3 with a 20-85% yield as an off-white solid.

### Synthesis of Final Compound 5

A mixture of common intermediate 3 (1.0 eq), R₁-substituted alkylamine 4 (5.0 eq), Pd₂(dba)₃ (0.1 eq), Xphos (0.2 eq) and Cs₂CO₃ (2.0 eq) in toluene (4 mL) was stirred at 100° C. for 16 h under an atmosphere of N₂. The reaction mixture was filtered. The filtrate was diluted with 20 mL of water, extracted with 20 mL of EtOAc twice. The combined organic layers were washed with 20 mL of brine twice, dried over with Na₂SO₄, filtered and concentrated in vacuo to give a residue. The residue was purified by reverse phase (0.1% FA) and re-purified by prep-HPLC to the desired final compound in a 5-32% yield as a white solid.

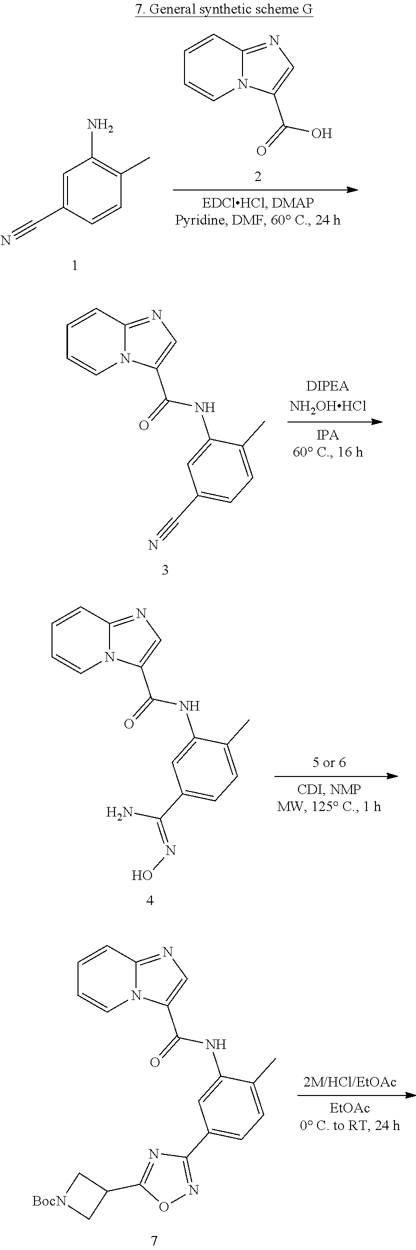

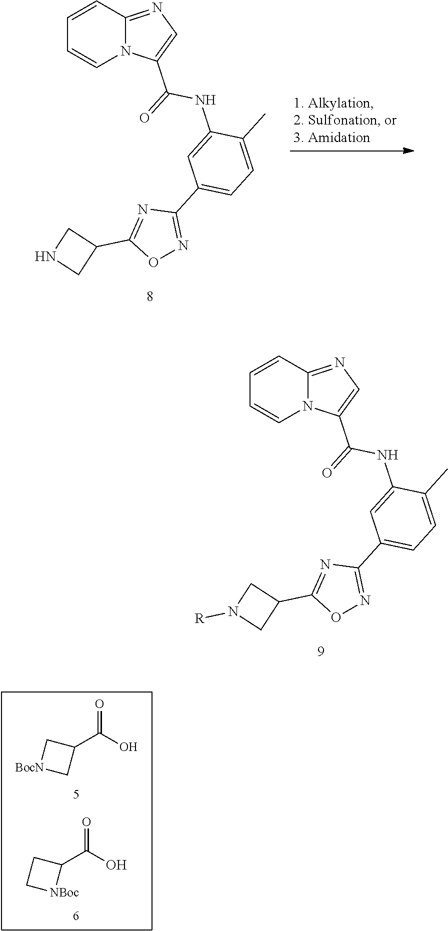

### Synthesis of Intermediate 3:

To a stirred solution of 3-amino-4-methylbenzonitrile 1 (1 eq), imidazo[1,2-a]pyridine-3-carboxylic acid 2 (1.05 eq), DMAP (1.3 eq) in DMF (0.1 M) was added pyridine (3 eq) into the reaction mixture at RT. After 10 min EDC.HCl (3 eq) was added. The reaction mixture was heated to 60° C. for 16 h. Upon heating, it slowly turned to clear brown solution. The reaction was monitored by TLC and LCMS. If SM was still observed an additional equivalent of EDC.HCl was added to the reaction mixture which was stirred for an additional 8 h at 60° C. The reaction mixture was then poured dropwise to ice water (1.5 L) and stirred for 30 min. The resulting solid was filtered, washed with water (200 mL) and hexane (500 mL) and dried under reduced pressure to afford N-(5-cyano-2-methylphenyl)imidazo[1,2-a]pyridine-3-carboxamide or intermediate 3 as off-white solid in a greater than 90% crude yield.

### Synthesis of Intermediate 4:

To a stirred solution of N-(5-cyano-2-methylphenyl)imidazo[1,2-a]pyridine-3-carboxamide or intermediate 3 (1 eq) in IPA (0.35 M) was added DIPEA (2 eq) and hydroxylamine hydrochloride (2 eq) sequentially at RT. The reaction mixture was then heated to 60° C. for 16 h. Progress of the reaction was monitored by TLC and LCMS. After completion of the reaction, the reaction mixture was cooled to RT. The resulting solids were collected by vacuum filtration, washed with 50% IPA & water, dried and triturated in EtOAc at 60° C. for 4 h. The obtained solid was filtered and dried under vacuum to afford common intermediate 4 in a 76% yield as an off-white solid.

### Synthesis of Intermediate 7:

To stirred solution of carboxylic acid 5 or 6 (1 eq) in NMP (0.03 M) was added 1′-carbonyldiimidazole (1 eq) at RT and allowed to stir for 10 min. After 20 min, intermediate 4 (0.5 eq) was added and the reaction mixture was stirred for 30 min at RT. The reaction mixture was subjected to microwave irradiation at 125° C. in a microwave reactor for 20 min. Progress of the reaction was monitored by TLC and LCMS. The reaction mixture was poured into ice water. The precipitate obtained was filtered and dried under reduced pressure to get crude. The obtained crude material was purified by prep-HPLC purification, to give desired final compound 6 in a 20-80% yield.

### Synthesis of Common Intermediate 8:

To a stirred solution of Boc-protected intermediate 7 (1 eq) in DCM (0.05 M) was added 2M HCl in EtOAc (20 eq) at 0° C. and allowed to stirred RT for 2 h. Progress of the reaction was monitored by TLC. After completion of the reaction, the reaction mixture was evaporated under vacuum to obtain crude material. The obtained crude material purified by prep-HPLC purification. Prep fractions were lyophilized to afford free base intermediate 8 in quantitative yield.

### Representative Alkylation:

To a stirred solution of intermediate 8, (1.0 eq), DIEA (20 eq) in DMF (3.00 mL) was added the R-substituted alkyl halide (20 eq). The mixture was stirred at 40° C. for 16 h. On completion, the reaction mixture was poured into 30 mL of water and extracted with 15 mL EtOAc by three times. The combined organic phase was washed with saturated 30 mL of brine, dried over with anhydrous Na₂SO₄, filtered and concentrated in vacuum to give a residue. The residue was purified by prep-HPLC purification to give the final product 9 in a 10-30% yield as a yellow solid.

### Representative Amidation:

To stirred solution of intermediate 8, (1.0 eq) and 2-hydroxyacetic acid (1.3 eq) in DMF (15 mL) was added DIPEA (3.0 eq) followed by HATU (1.8 eq) and stirred at RT for 16 h. The reaction progress was monitored by TLC. The reaction mixture was diluted with water and extracted with EtOAc (3×75 mL). The combined organic layers were washed with brine solution, dried over Na₂SO₄, filtered and concentrated to afford off white sticky residue. The obtained crude material was purified by column chromatography (SiO₂, DCM:MeOH, 5:95). The desired product was isolated in 43% yield.

### Synthesis of Compound 3.

To a stirred solution of acid 1 (1.0 eq) in dry DCM (20 mL) was cooled to 0° C. and oxalyl chloride (2 eq) was added drop wise and followed by dry DMF (0.5 mL) and stirred at RT for 1.5 h and reaction mixture was concentrated under vacuum to obtain crude material. The obtained crude material was added to a stirred solution aniline 2 in pyridine (0.2 M) at 0° C. The resulting reaction mixture was stirred at RT for 2 h. Progress of the reaction was monitored by TLC. After completion of the reaction, the reaction mixture was diluted with H₂O (20 mL) and extracted with EtOAc (3×20 mL). The organic layer dried over Na₂SO₄, concentrated to obtain crude material. The obtained crude material was purified by column chromatography (SiO₂, hexane:EtOAc, 2:3) 60-65% EtOAc in Hexane as eluent to afford intermediate compound 3 in a 20-65% yield as an off-white solid.

### Synthesis of Compound 4.

To a stirred solution of compound 3 (1 eq) in toluene (10 mL) was added Lawesson's reagent (1.2 eq) at RT. The resulting reaction mixture in microwave vial was irradiated in microwave reactor at 150° C. for 1 h. Progress of the reaction was monitored by TLC analysis. After completion of the reaction, the reaction mixture was diluted with H₂O (50 mL) and extracted with EtOAc (2×50 mL). The organic layer dried over Na₂SO₄, concentrated to obtain crude material. The obtained crude material was purified by column chromatography (SiO₂, PE:EtOAc, 1:2) to obtain impure material. The impure material further re-purified by prep-HPLC Purification. Prep fractions were lyophilized to afford the desired thioamide in a 5-35% yield as off-white solid.

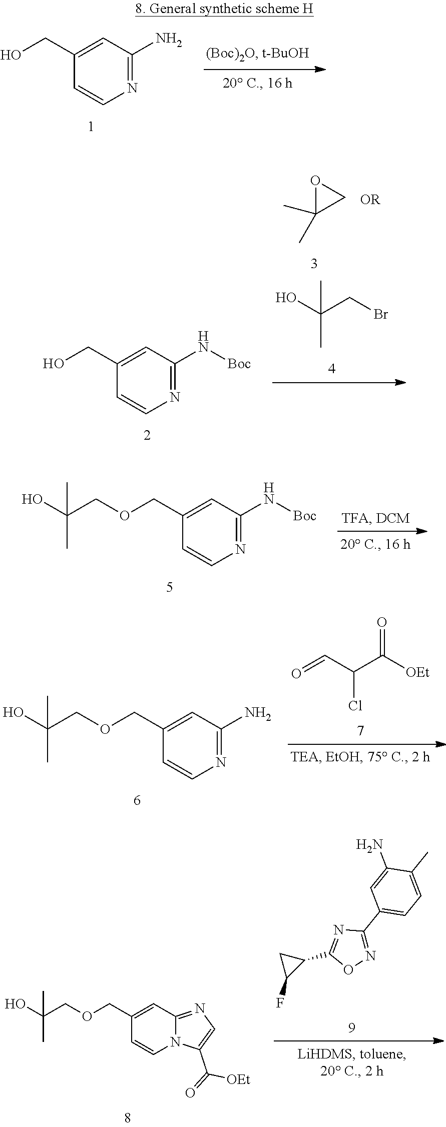

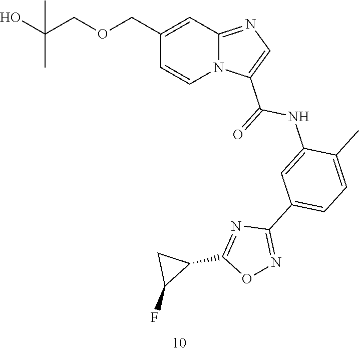 [SMILES: Cc1ccc(-c2noc([C@H]3C[C@@H]3F)n2)cc1NC(=O)c1cnc2cc(COCC(C)(C)O)ccn12]

### Synthesis of Intermediate 2:

To a solution of compound 1 (1.0 eq) in t-BuOH (0.1M) was added Boc₂O (1.5 eq). The mixture was stirred at 20° C. for 16 hours. The reaction was monitored by TLC and LCMS and was quenched when compound 1 was completely consumed. The reaction mixture was concentrated under reduced pressure to remove t-BuOH. The residue was purified by column chromatography (SiO₂, PE:EtOAc, 1:0 to 1:1). Intermediate 2 was obtained as a white solid in a 37% yield.

### Synthesis of Intermediate 5:

To a solution of compound 2 (1.0 eq) and either epoxide 3 or alkyl halide 4 (2.0 eq) in THF (0.2M) was added TEA (3.0 eq) by slow addition. The mixture was stirred at 70° C. for 4 hours. The reaction was monitored by TLC and LCMS and was quenched when compound 2 was completely consumed. The mixture was diluted with water (1500 mL) and extracted with EtOAc (1500 mL×2). The combined organic layers were dried over anhydrous sodium sulfate, filtered and concentrated under reduced pressure to give a residue. The crude product was used to the next step without further purification. Compound 5 was obtained as crude black oil, and carried forward without purification and assumed quantitative yield.

### Synthesis of Intermediate 6:

To a solution of compound 5 (1.0 eq) in DCM (0.5M) was added TFA (400 mL). The mixture was stirred at 20° C. for 16 hours. The reaction was monitored by TLC and LCMS and was quenched when compound 5 was completely consumed. The reaction mixture was concentrated under reduced pressure to remove DCM and TFA. The residue was used without further purification. Intermediate 6 (crude, TFA present) was obtained as crude yellow oil and carried forwards without purification and assume quantitative yield.

### Synthesis of Intermediate 8:

To a solution of 6 (1.0 eq, TFA salt) and chloroaldehyde 7 (1.5 eq) in EtOH (0.1 M) was added TEA (5.0 eq). The mixture was stirred at 70° C. for 4 hours. The reaction was monitored by TLC and LCMS, and was quenched when compound 6 was completely consumed. The reaction mixture was concentrated under reduced pressure to remove EtOH. The mixture was diluted with water (1500 mL) and extracted with EtOAc (2000 mL×3). The combined organic layers were dried over anhydrous sodium sulfate, filtered and concentrated under reduced pressure to give a residue. The residue was purified by column chromatography (SiO₂, PE:EtOAc, 1:0 to 3:2). Intermediate 8 was obtained as yellow oil in a 42% yield.

### Synthesis of Final Compound 10:

Intermediate 8 (1.0 eq) and common intermediate 9 (1.2 eq) in toluene (0.1M) was added LiHMDS (3 eq) at 20° C. The mixture was stirred at 20° C. for 2 hours. LCMS showed ˜12% of compound 8 and ˜₁₃% of compound 9 remained, with a majority of desired product present. The reaction mixture was quenched by addition of aq NH₄Cl (1000 mL) at 20° C., extracted with EtOAc/2-Me-THF (1:1) 2000 mL (1000 mL×2). The combined organic layers were dried over Na₂SO₄, filtered and concentrated under reduced pressure to give a residue. The residue was purified by column chromatography (SiO₂, PE/EtOAc, 1:0 to 0:1). Final compound 10 was obtained as an off-white solid in a 47% yield.

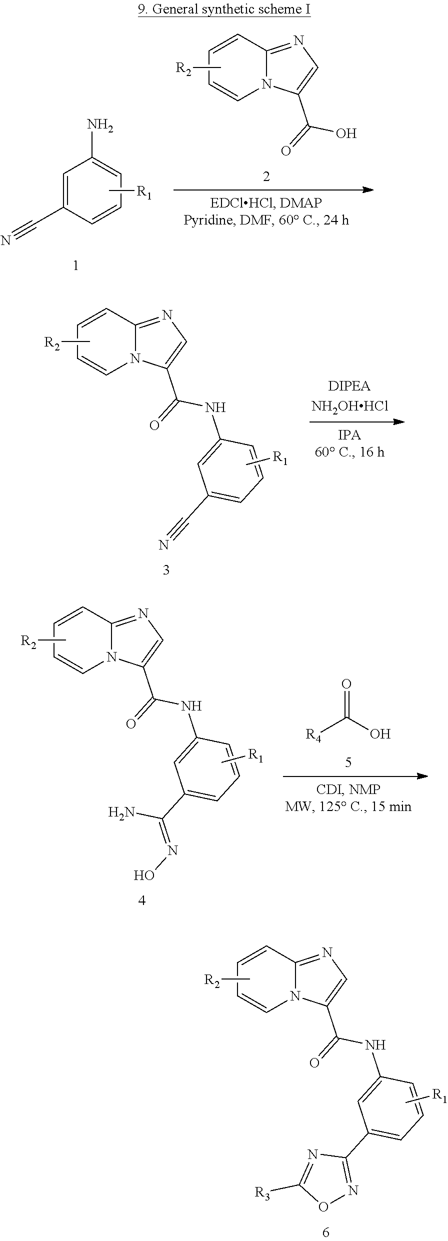

### Synthesis of Intermediate 3:

To a stirred solution of R₁-substituted 3-amino-benzonitrile 1 (1 eq), the corresponding imidazopyridine acid 2 (1.05 eq), DMAP (1.3 eq) in DMF (0.1 M) was added pyridine (3 eq) into the reaction mixture at rt. After 10 min EDC.HCl (3 eq) was added (reaction mixture does not turn to a clear solution, looks like thick precipitate). The reaction mixture was heated to 60° C. for 16 h. Upon heating, it slowly turned to clear brown solution. The reaction was monitored by TLC and LCMS. If SM was still observed an additional equivalent of EDC.HCl was added to the reaction mixture and allowed to stir for an additional 8 h at 60° C. After completion of the reaction, the reaction mixture was poured into ice-cold water (1.5 L) and stirred for 30 min. The resulting solid was filtered, washed with water (200 mL) and hexane (500 mL) and dried under reduced pressure to afford intermediate 3 as off-white solid in a greater than 90% crude yield.

### Synthesis of Intermediate 4:

To a stirred solution of intermediate 3 (1 eq) in IPA (0.35 M) was added DIPEA (2 eq) and hydroxylamine hydrochloride (2 eq) sequentially at RT. The reaction mixture was then heated to 60° C. for 16 h. Progress of the reaction was monitored by TLC and LCMS. After completion of the reaction, the reaction mixture was cooled to rt. The resulting solid were collected by vacuum filtration, washed with 50% IPA & water, dried and triturated in EtOAc at 60° C. for 4 h. The obtained solid was filtered and dried under vacuum to afford (Z)—N-(5-(N′-hydroxycarbamimidoyl)- common intermediate 4 in a 40-76% yield as an off-white solid.

### Synthesis of Intermediate 6:

To stirred solution of carboxylic acid 2 (1 eq) in NMP (0.03 M) was added 1′-Carbonyldiimidazole (1 eq) at RT and stirred for 20 min. After 20 min, intermediate 4 (0.5 eq) was added and the reaction mixture was stirred for 30 min at RT. The reaction mixture was subjected to microwave irradiation at 125° C. in a microwave reactor for 15-20 min. Progress of the reaction was monitored by TLC and LCMS. The reaction mixture was poured into ice water. The precipitate obtained was filtered and dried under reduced pressure to get crude. The obtained crude material was purified by prep-HPLC purification, to give desired final compound 6 in a 20-80% yield.

### Example 2—Preparation of N-[5-[5-[(1R,2S)-2-fluorocyclopropyl]-1,2,4-]-7-[(1S*)-1-(2-hydroxyethoxadiazol-3-yl]-2-methyl-phenyloxy)ethyl]imidazo[1,2-a]pyridine-3-carboxamide (I-450) and N-[5-[5-[(1R,2S)-2-fluorocyclopropyl]-1,2,4-oxadiazol-3-yl]-2-methyl-phenyl]-7-[(1R*)-1-(2-hydroxyethoxy)ethyl]imidazo[1,2-a]pyridine-3-carboxamide (I-451)

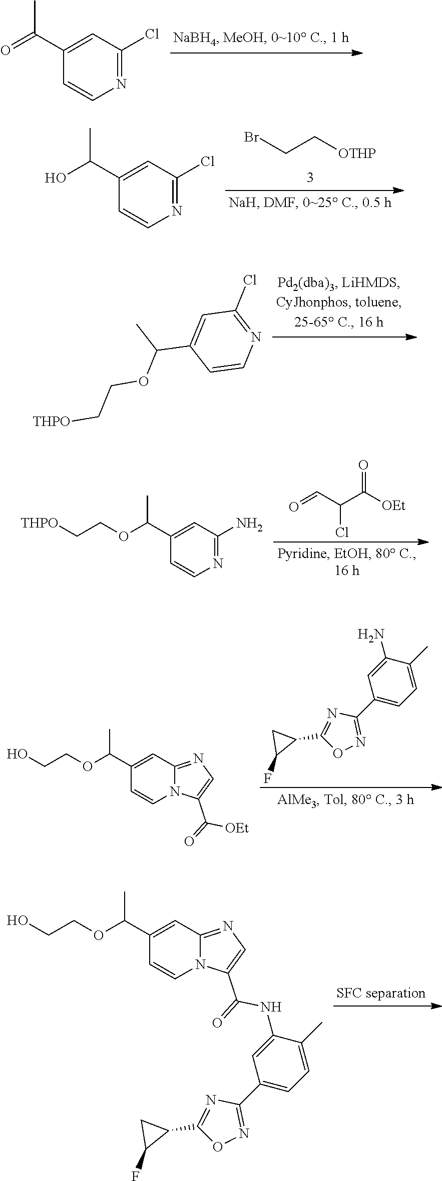

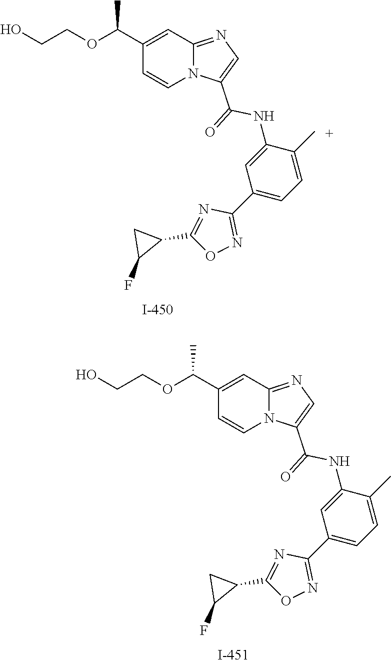

Step 1: To a mixture of 1-(2-chloro-4-pyridyl)ethanone (5 g, 32.1 mmol, 1 eq) in MeOH (50 mL) was added NaBH₄ (1.44 g, 38.1 mmol, 1.18 eq) at 0° C. in portions, the reaction mixture was stirred at 0° C. for 0.5 hr. Then the mixture was allowed to warm to 10° C. under N₂ for 0.5 hr. The mixture was quenched with sat.aq.NH₄Cl (2 mL) and concentrated to give a residue. The residue was washed with H₂O (15×3 mL) and extracted with ethyl acetate (30 mL). The organic layer was washed with brine (15 mL), dried over Na₂SO₄, filtered and concentrated to give a crude product 1-(2-chloro-4-pyridyl)ethanol (4.88 g, 30.9 mmol, 96%) as a yellow oil. ¹H NMR (400 MHz, DMSO-d₆) δ=8.34 (d, J=5.2 Hz, 1H), 7.45 (s, 1H), 7.38 (d, J=4.4 Hz, 1H), 5.53 (d, J=4.4 Hz, 1H), 4.79-4.72 (m, 1H), 1.33 (d, J=6.4 Hz, 3H)

Step 2: To a mixture of 1-(2-chloro-4-pyridyl)ethanol (0.2 g, 1.27 mmol, 1 eq) in DMF (5 mL) was added NaH (55.8 mg, 1.40 mmol, 60% purity, 1.1 eq) at 0° C. 2-(2-bromoethoxy)tetrahydropyran (278 mg, 1.33 mmol, 201 μL, 1.05 eq) was added and stirred at 25° C. for 0.5 hr. The mixture was quenched with sat.aq.NH₄Cl (2 mL) and concentrated to give a residue. The mixture was purified by column chromatography (SiO₂, petroleum ether:ethyl acetate=1:0-3:1) to give 2-chloro-4-[1-(2-tetrahydropyran-2-yloxyethoxy)ethyl]pyridine (0.2 g, 699 μmol, 55.2% yield) as a colorless oil. ¹H NMR (400 MHz, DMSO-d₆) δ=8.38 (d, J=5.2 Hz, 1H), 7.49 (s, 1H), 7.39 (d, J=5.2 Hz, 1H), 4.63-4.53 (m, 2H), 3.75-3.67 (m, 2H), 3.67-3.66 (m, 1H), 3.59-3.50 (m, 2H), 3.47-3.40 (m, 2H), 1.53-1.43 (m, 5H), 1.35 (d, J=6.4 Hz, 3H)

Step 3: A mixture of 2-chloro-4-[1-(2-tetrahydropyran-2-yloxyethoxy)ethyl]pyridine (1 g, 3.50 mmol, 1 eq), dicyclohexyl-(2-phenylphenyl)phosphane (245 mg, 700 μmol, 0.2 eq) and Pd₂(dba)₃ (320 mg, 350 μmol, 0.1 eq) in ToI. (20 mL) was stirred at 25° C. for 15 min. LiHMDS (1 M, 5.25 mL, 1.5 eq) was added and stirred at 65° C. for 15 hr. The mixture was quenched with sat.aq.NH₄Cl (2 mL), diluted with H₂O (30 mL) and extracted with ethyl acetate (30×2 mL). The organic layer was washed with brine (30 mL), dried over Na₂SO₄, filtered and concentrated to give a residue. The residue was purified by column chromatography (SiO₂, petroleum ether:ethyl acetate=10:1˜1:1) to give 4-[1-(2-tetrahydropyran-2-yloxyethoxy)ethyl]pyridin-2-amine (0.4 g, 1.50 mmol, 42.9% yield) as a yellow oil. ¹H NMR (400 MHz, DMSO-d₆) δ=7.83 (d, J=5.2 Hz, 1H), 6.43-6.39 (m, 1H), 6.35 (s, 1H), 5.83 (s, 2H), 4.55 (d, J=9.4 Hz, 1H), 4.33-4.25 (m, 1H), 3.77-3.63 (m, 2H), 3.52-3.35 (m, 4H), 1.77-1.65 (m, 1H), 1.63-1.54 (m, 1H), 1.51-1.37 (m, 4H), 1.26 (d, J=6.5 Hz, 3H)

Step 4: To a mixture of 4-[1-(2-tetrahydropyran-2-yloxyethoxy)ethyl]pyridin-2-amine (2 g, 7.51 mmol, 1 eq) and Pyridine (890 mg, 11.2 mmol, 909 μL, 1.5 eq) in EtOH (20 mL) was added ethyl 2-chloro-3-oxo-propanoate (1.36 g, 9.01 mmol, 1.2 eq). The mixture was stirred at 80° C. for 16 hr. The mixture was concentrated to give a residue. The mixture was purified by reversed-phase HPLC(0.1% NH₃.H₂O) to give ethyl 7-[1-(2-hydroxyethoxy)ethyl]imidazo[1,2-a]pyridine-3-carboxylate (0.85 g, 3.05 mmol, 40.6% yield) as a yellow oil. ¹H NMR (400 MHz, DMSO-d₆) δ=9.20 (d, J=7.2 Hz, 1H), 8.28 (s, 1H), 7.74 (s, 1H), 7.28-7.22 (m, 1H), 4.68-4.59 (m, 2H), 4.41-4.32 (m, 2H), 3.55-3.48 (m, 2H), 3.44-3.38 (m, 1H), 3.33-3.29 (m, 1H), 1.40 (d, J=6.4 Hz, 3H), 1.35 (t, J=7.1 Hz, 3H)

Step 5: To a mixture of 5-[5-[(1R,2S)-2-fluorocyclopropyl]-1,2,4-oxadiazol-3-yl]-2-methyl-aniline (587 mg, 2.52 mmol, 1 eq) and ethyl 7-[1-(2-hydroxyethoxy)ethyl]imidazo[1,2-a]pyridine-3-carboxylate (700 mg, 2.52 mmol, 1 eq) in ToI. (10 mL) was added AlMe₃ (2 M, 3.14 mL, 2.5 eq). The mixture was stirred at 80° C. for 3 hr. The reaction mixture was quenched by 1M HCl (10 mL) at 0° C. The mixture was diluted with water (50 mL) and basified with saturated NaHCO₃ aqueous until pH=7. The mixture was extracted with ethyl acetate (20 mL×3). The combined organic layers were washed with brine (50 mL×3). The organic layer was dried over anhydrous sodium sulfate and filtered. The filtrate was concentrated in vacuo. The residue was purified by prep-HPLC (column: Phenomenex luna C18 150*40 mm*15 um; mobile phase: [water(FA)-ACN]; gradient: 17%-47% B over 11 min) to give N-[5-[5-[(1R,2S)-2-fluorocyclopropyl]-1,2,4-oxadiazol-3-yl]-2-methyl-phenyl]-7-[1-(2-hydroxyethoxy)ethyl]imidazo[1,2-a]pyridine-3-carboxamide (600 mg, 1.07 mmol, 42.5% yield, 99.7% purity) as a white solid. ¹H NMR (400 MHz, DMSO-d₆) δ=10.01 (s, 1H), 9.41 (d, J=7.2 Hz, 1H), 8.57 (s, 1H), 8.03 (d, J=1.2 Hz, 1H), 7.81-7.76 (m, 1H), 7.71 (s, 1H), 7.49 (d, J=8.0 Hz, 1H), 7.20-7.13 (m, 1H), 5.40-5.18 (m, 1H), 4.67-4.59 (m, 2H), 3.57-3.50 (m, 2H), 3.45-3.38 (m, 1H), 3.11-3.01 (m, 1H), 2.36 (s, 3H), 2.02-1.88 (m, 1H), 1.65-1.54 (m, 1H), 1.41 (d, J=6.4 Hz, 3H)

Step 6: N-[5-[5-[(1R,2S)-2-fluorocyclopropyl]-1,2,4-oxadiazol-3-yl]-2-methyl-phenyl]-7-[1-(2-hydroxyethoxy)ethyl]imidazo[1,2-a]pyridine-3-carboxamide (600 mg, 1.07 mmol) was purified by SFC (Column: Chiralpak AD-3 50*4.6 mm I.D., 3 μm Mobile phase: Phase A for CO2, and Phase B for EtOH+CAN (0.05% DEA)); Gradient elution: 60% EtOH+CAN (0.05% DEA) in CO2, Flow rate: 3 mL/min; Detector: PDA; Column Temp: 35° C.; Back Pressure: 100 Bar) to give N-[5-[5-[(1R,2S)-2-fluorocyclopropyl]-1,2,4-]-7-[(1S*)-1-(2-hydroxyethoxadiazol-3-yl]-2-methyl-phenyloxy)ethyl]imidazo[1,2-a]pyridine-3-carboxamide (283 mg, 608 μmol, 47.18% yield) and N-[5-[5-[(1R,2S)-2-fluorocyclopropyl]-1,2,4-oxadiazol-3-yl]-2-methyl-phenyl]-7-[(1R*)-1-(2-hydroxyethoxy)ethyl]imidazo[1,2-a]pyridine-3-carboxamide (289 mg, 621 μmol, 48.1% yield). ¹H NMR (400 MHz, DMSO-d6) δ=10.01 (s, 1H), 9.41 (d, J=7.2 Hz, 1H), 8.57 (s, 1H), 8.03 (d, J=1.6 Hz, 1H), 7.78 (m, 1H), 7.71 (s, 1H), 7.49 (d, J=8.0 Hz, 1H), 7.17 (m, 1H), 5.42-5.16 (m, 1H), 4.72-4.56 (m, 2H), 3.57-3.49 (m, 2H), 3.46-3.39 (m, 1H), 3.13-3.01 (m, 1H), 2.36 (s, 3H), 2.02-1.87 (m, 1H), 1.59 (m, 1H), 1.41 (d, J=6.4 Hz, 3H). MS (ESI): m/z for C₂₄H₂₄FN₅O₄[M+H]+ calcd. 466.2, [M+H]+ found. 466.1.

### Example 3, Preparation of N-[5-[5-[(1R,2S)-2-fluorocyclopropyl]-1,2,4-oxadiazol-3-yl]-2-methyl-phenyl]-7-(4-hydroxy-4-methyl-pentyl)imidazo[1,2-a]pyridine-3-carboxamide (I-452)

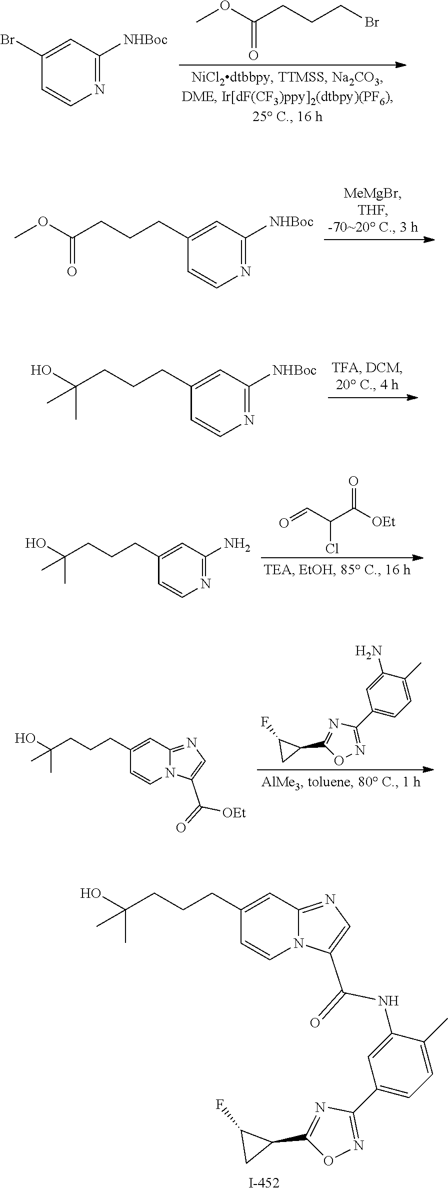

Step 1: To a mixture of tert-butyl N-(4-bromo-2-pyridyl)carbamate (3 g, 10.9 mmol, 1 eq), methyl 4-bromobutanoate (2.58 g, 14.2 mmol, 1.3 eq), Ir[dF(CF₃)ppy]₂(dtbpy)(PF₆) (123 mg, 109 μmol, 0.01 eq), TTMSS (2.73 g, 10.9 mmol, 3.39 mL, 1 eq) and Na₂CO₃ (2.33 g, 21.9 mmol, 2 eq) in DME (2 mL) was added NiCl₂.dtbbpy (21.8 mg, 54.9 μmol, 0.005 eq). The reaction mixture was stirred at 25° C. for 16 hrs under nitrogen atmosphere and irradiated with a 455 nm blue LED. The reaction mixture was filtered. The filtrate was diluted with water (200 mL) and extracted with ethyl acetate (100 mL×3). The combined organic layers were washed with brine (200 mL×3). The organic layer was dried over anhydrous sodium sulfate and filtered. The filtrate was concentrated in vacuo. The residue was purified by column chromatography (SiO₂, Petroleum ether:ethyl acetate=5:1) to give methyl 4-[2-(tert-butoxycarbonylamino)-4-pyridyl]butanoate (2.68 g, 9.10 mmol, 82.8% yield) as a yellow solid. ¹H NMR (400 MHz, DMSO-d₆) δ: 9.65 (s, 1H), 8.11 (d, J=5.2 Hz, 1H), 7.65 (s, 1H), 6.86 (dd, J=1.6, 5.2 Hz, 1H), 3.59 (s, 3H), 2.61-2.54 (m, 2H), 2.33 (t, J=7.2 Hz, 2H), 1.86-1.77 (m, 2H), 1.47 (s, 9H)

Step 2: To a mixture of methyl 4-[2-(tert-butoxycarbonylamino)-4-pyridyl]butanoate (2.58 g, 8.77 mmol, 1 eq) in THE (25 mL) was added MeMgBr (3 M, 8.77 mL, 3 eq) at −70° C. The reaction mixture was stirred at 20° C. for 3 hrs under nitrogen atmosphere. The reaction mixture was quenched by addition saturated NH₄Cl (10 mL) at 0° C. The mixture was diluted with water (100 mL) and extracted with ethyl acetate (50 mL×3). The combined organic layers were washed with brine (100 mL×3). The organic layer was dried over anhydrous sodium sulfate and filtered. The filtrate was concentrated in vacuo. The residue was purified by column chromatography (SiO₂, Petroleum ether:ethyl acetate=3:1) to give tert-butyl N-[4-(4-hydroxy-4-methyl-pentyl)-2-pyridyl]carbamate (1.81 g, 6.15 mmol, 70.1% yield) as a white solid. ¹H NMR (400 MHz, DMSO-d₆) δ: 9.61 (s, 1H), 8.10 (d, J=5.2 Hz, 1H), 7.64 (s, 1H), 6.86 (d, J=5.2 Hz, 1H), 4.11 (s, 1H), 2.56-2.52 (m, 2H), 1.66-1.54 (m, 2H), 1.47 (s, 9H), 1.39-1.32 (m, 2H), 1.05 (s, 6H)

Step 3: To a mixture of tert-butyl N-[4-(4-hydroxy-4-methyl-pentyl)-2-pyridyl]carbamate (700 mg, 2.38 mmol, 1 eq) in DCM (6 mL) was added TFA (3.07 g, 26.9 mmol, 2 mL, 11.3 eq). The reaction mixture was stirred at 20° C. for 4 hrs. The reaction mixture was concentrated in vacuo to give 5-(2-amino-4-pyridyl)-2-methyl-pentan-2-ol (700 mg, 2.27 mmol, 95.4% yield, TFA) as a yellow oil. MS (ESI): m/z for C₁₁H₁₈N₂O [M+H]+ calcd. 195.1, [M+H]+ found. 195.3.

Step 4: To a mixture of 5-(2-amino-4-pyridyl)-2-methyl-pentan-2-ol (700 mg, 2.27 mmol, 1 eq, TFA) and ethyl 2-chloro-3-oxo-propanoate (512 mg, 3.41 mmol, 1.5 eq) in EtOH (10 mL) was added triethylamine (459 mg, 4.54 mmol, 632 μL, 2 eq). The reaction mixture was stirred at 85° C. for 16 hrs under nitrogen atmosphere. The reaction mixture was concentrated in vacuo. The residue was purified by column chromatography (SiO₂, petroleum ether:ethyl acetate=3:1) to give ethyl 7-(4-hydroxy-4-methyl-pentyl)imidazo[1,2-a]pyridine-3-carboxylate (530 mg, 1.83 mmol, 80.3% yield) as a yellow oil. ¹H NMR (400 MHz, DMSO-d₆) δ: 9.12 (d, J=7.2 Hz, 1H), 8.23 (s, 1H), 7.59 (s, 1H), 7.14 (dd, J=1.2, 7.2 Hz, 1H), 4.35 (q, J=7.2 Hz, 2H), 4.15-4.08 (m, 1H), 2.69 (t, J=7.2 Hz, 2H), 1.74-1.61 (m, 2H), 1.38 (dd, J=3.6, 8.0 Hz, 2H), 1.34 (t, J=7.2 Hz, 3H), 1.06 (s, 6H)

Step 5: To a mixture of ethyl 7-(4-hydroxy-4-methyl-pentyl)imidazo[1,2-a]pyridine-3-carboxylate (500 mg, 1.72 mmol, 1 eq) and 5-[5-[(1R,2S)-2-fluorocyclopropyl]-1,2,4-oxadiazol-3-yl]-2-methyl-aniline (401 mg, 1.72 mmol, 1 eq) in toluene (5 mL) was added AlMe₃ (2 M, 2.15 mL, 2.5 eq). The reaction mixture was stirred at 80° C. for 1 hr. The reaction mixture was quenched by saturated NH₄Cl (10 mL) at 0° C. The mixture was diluted with water (100 mL) and extracted with ethyl acetate (50 mL×3). The combined organic layers were washed with brine (100 mL×3). The organic layer was dried over anhydrous sodium sulfate and filtered. The filtrate was concentrated in vacuo. The residue was purified by Prep-HPLC (column: Waters Xbridge Prep OBD C18 150*40 mm*10 um; mobile phase: [water (NH₄HCO₃)-ACN]; gradient: 30%-60% B over 15 min) to give N-[5-[5-[(1R,2S)-2-fluorocyclopropyl]-1,2,4-oxadiazol-3-yl]-2-methyl-phenyl]-7-(4-hydroxy-4-methyl-pentyl)imidazo[1,2-a]pyridine-3-carboxamide (I-452) (276 mg, 575 μmol, 33.4% yield, 99.2% purity) as a white solid. ¹H NMR (400 MHz, DMSO-d₆) δ: 9.95 (s, 1H), 9.33 (d, J=7.2 Hz, 1H), 8.52 (s, 1H), 8.02 (d, J=1.6 Hz, 1H), 7.77 (dd, J=1.6, 8.0 Hz, 1H), 7.55 (s, 1H), 7.47 (d, J=8.0 Hz, 1H), 7.06 (dd, J=1.6, 7.2 Hz, 1H), 5.39-5.18 (m, 1H), 4.12 (s, 1H), 3.11-3.00 (m, 1H), 2.69 (t, J=7.2 Hz, 2H), 2.35 (s, 3H), 2.01-1.87 (m, 1H), 1.75-1.64 (m, 2H), 1.63-1.53 (m, 1H), 1.44-1.34 (m, 2H), 1.07 (s, 6H). MS (ESI): m/z for C₂₆H₂₈FN₅O₃[M+H]+ calcd. 478.2, [M+H]+ found. 478.3.

### Example 4—Preparation of N-[5-[5-[(1R,2S)-2-fluorocyclopropyl]-1,2,4-oxadiazol-3-yl]-2-methyl-phenyl]-6-(2-hydroxyethoxymethyl)imidazo[1,2-a]pyridine-3-carboxamide (I-453)

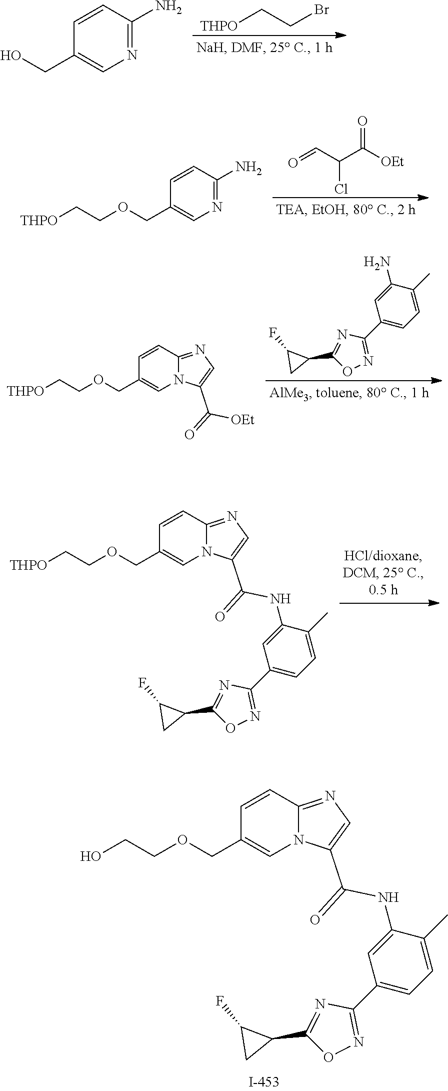

Step 1: To a mixture of (6-amino-3-pyridyl)methanol (1 g, 8.06 mmol, 1 eq) in DMF (10 mL) was added NaH (644 mg, 16.1 mmol, 60% purity, 2 eq) at 0° C. and stirred at 30 min, then 2-(2-bromoethoxy)tetrahydropyran (2.11 g, 10.0 mmol, 1.52 mL, 1.25 eq) was added and stirred at 25° C. for 1 hr. The reaction mixture was quenched by sat.aq.NH₄Cl (10 mL). The reaction mixture was diluted with H₂O (15 mL) and extracted with ethyl acetate (15 mL×3). The combined organic layers were washed with brine (30 mL), dried over Na₂SO₄. The mixture was filtered and concentrated in vacuo. to give 5-(2-tetrahydropyran-2-yloxyethoxymethyl)pyridin-2-amine (2 g, 7.93 mmol, 98% yield) was obtained as yellow solid. MS (ESI): m/z for C₁₅H₂₄N₂O₃ [M+H]+ calcd. 253.1, [M+H]+ found. 253.2.

Step 2: To a mixture of 5-(2-tetrahydropyran-2-yloxyethoxymethyl)pyridin-2-amine (1.8 g, 7.13 mmol, 1 eq) and triethylamine (1.08 g, 10.7 mmol, 1.49 mL, 1.5 eq) in EtOH (30 mL) was added ethyl 2-chloro-3-oxo-propanoate (1.07 g, 7.13 mmol, 1 eq). The mixture was stirred at 80° C. for 2 hr. The reaction mixture was diluted with H2O 100 mL and extracted with ethyl acetate (30 mL×3). The combined organic layers were washed with brine (30 mL) dried over Na₂SO₄. The mixture was filtered and concentrated in vacuo. The crude product was purified by reversed-phase HPLC (0.1% NH₃·H2O) to give ethyl 6-(2-tetrahydropyran-2-yloxyethoxymethyl)imidazo[1,2-a]pyridine-3-carboxylate (0.4 g, 1.15 mmol, 16.0% yield) was obtained as white oil. ¹H NMR (400 MHz, DMSO-d₆) δ=9.23 (s, 1H), 8.29 (s, 1H), 7.81 (d, J=9.2 Hz, 1H), 7.54 (m, 1H), 4.66 (s, 2H), 4.60 (t, J=3.6 Hz, 1H), 4.37 (q, J=7.2 Hz, 2H), 3.82-3.72 (m, 2H), 3.68-3.64 (m, 2H), 3.60-3.51 (m, 1H), 3.45-3.38 (m, 1H), 1.74-1.56 (m, 2H), 1.53-1.40 (m, 4H), 1.35 (t, J=7.2 Hz, 3H).

Step 3: To a mixture of ethyl 6-(2-tetrahydropyran-2-yloxyethoxymethyl)imidazo[1,2-a]pyridine-3-carboxylate (210 mg, 602 μmol, 1 eq) and 5-[5-[(1R,2S)-2-fluorocyclopropyl]-1,2,4-oxadiazol-3-yl]-2-methyl-aniline (140 mg, 602 μmol, 1 eq) in ToI. (5 mL) was added AlMe₃ (2 M, 753 μL, 2.5 eq). The mixture was stirred at 80° C. for 1 hr. The reaction mixture was quenched by HCl (1 M). The mixture was diluted with water (20 mL) and extracted with ethyl acetate (10 mL×3). The combined organic layers were washed with brine (30 mL×3). The organic layer was dried over anhydrous sodium sulfate and filtered. The filtrate was concentrated in vacuo to give N-[5-[5-[(1R,2S)-2-fluorocyclopropyl]-1,2,4-oxadiazol-3-yl]-2-methyl-phenyl]-6-(2-tetrahydropyran-2-yloxyethoxymethyl)imidazo[1,2-a]pyridine-3-carboxamide (300 mg, 560 μmol, 92.9% yield) as red oil. MS (ESI): m/z for C₂₈H₃₀FN₅O₅ [M+H]+ calcd. 536.2, [M+H]+ found 536.3.

Step 4: To a mixture of N-[5-[5-[(1R,2S)-2-fluorocyclopropyl]-1,2,4-oxadiazol-3-yl]-2-methyl-phenyl]-6-(2-tetrahydropyran-2-yloxyethoxymethyl)imidazo[1,2-a]pyridine-3-carboxamide (300 mg, 560 μmol, 1 eq) in DCM (5 mL) was added HCl/dioxane (4 M, 1 mL, 7.14 eq). The mixture was stirred at 25° C. for 0.5 hr. The mixture was diluted with water (20 mL) and extracted with ethyl acetate (10 mL×3). The combined organic layers were washed with brine (30 mL×3). The organic layer was dried over anhydrous sodium sulfate and filtered. The filtrate was concentrated in vacuo. The crude product was purified by reversed-phase HPLC(column: YMC-Actus Triart C18 150*30 mm*7 um; mobile phase: [water(FA)-ACN]; gradient: 25%-55% B over 10 min) to give N-[5-[5-[(1R,2S)-2-fluorocyclopropyl]-1,2,4-oxadiazol-3-yl]-2-methyl-phenyl]-6-(2-hydroxyethoxymethyl)imidazo[1,2-a]pyridine-3-carboxamide (I-453) (169 mg, 331 μmol, 59.1% yield, 97% purity, FA) as white solid. ¹H NMR (400 MHz, DMSO-d₆) δ=10.04 (s, 1H), 9.44 (s, 1H), 8.58 (s, 1H), 8.04 (d, J=1.6 Hz, 1H), 7.82-7.75 (m, 2H), 7.54-7.47 (m, 2H), 5.42-5.18 (m, 1H), 4.76-4.65 (m, 1H), 4.60 (s, 2H), 3.55 (d, J=4.4 Hz, 2H), 3.52 (d, J=4.4 Hz, 2H), 3.12-3.01 (m, 1H), 2.36 (s, 3H), 2.04-1.90 (m, 1H), 1.59 (m, 1H). MS (ESI): m/z for C₂₃H₂₂FN₅O₄[M+H]+ calcd. 452.1, [M+H]+ found 452.2.

### Example 5—Preparation of N-[5-[5-[(1R,2S)-2-fluorocyclopropyl]-1,2,4-oxadiazol-3-yl]-2-methyl-phenyl]-7-(2-methylsulfonylethoxymethyl)imidazo[1,2-a]pyridine-3-carboxamide (I-454)

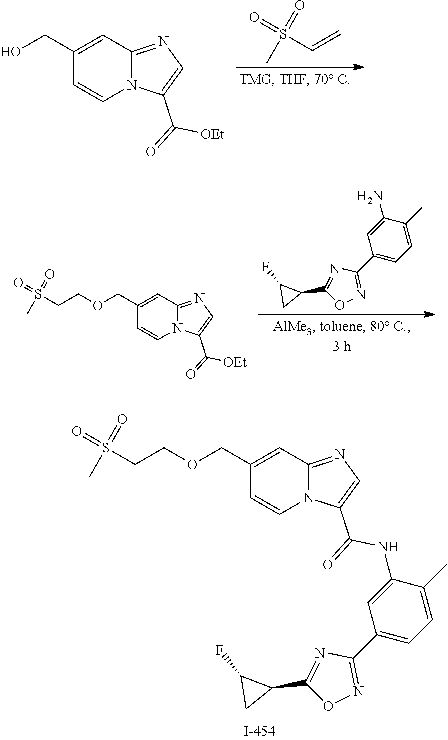

Step 1: To a solution of ethyl 7-(hydroxymethyl)imidazo[1,2-a]pyridine-3-carboxylate (0.7 g, 3.18 mmol) in dioxane (2.0 mL) was added 1,1,3,3-tetramethylguanidine (732 mg, 6.36 mmol, 799 μL) and 1-methylsulfonylethylene (1.01 g, 9.54 mmol, 835 μL). The mixture was stirred at 70° C. for 16 hours. After completion on LC-MS and TLC, the mixture was concentrated in vacuo to give a residue. The residue was purified by reverse phase (0.1% FA condition) to give compound ethyl 7-(2-methylsulfonylethoxymethyl)imidazo[1,2-a]pyridine-3-carboxylate (220 mg, 10.6% yield) as a yellow solid. ¹H NMR (400 MHz, CDCl₃) δ 9.31 (d, J=7.2 Hz, 1H), 8.31 (s, 1H), 7.76 (s, 1H), 7.04 (dd, J=1.6, 7.2 Hz, 1H), 4.69 (s, 2H), 4.43 (q, J=7.2 Hz, 2H), 4.05-4.00 (m, 2H), 3.33 (t, J=5.4 Hz, 2H), 3.04 (s, 3H), 1.43 (t, J=7.2 Hz, 3H)

Step 2: To a solution of ethyl 7-(2-methylsulfonylethoxymethyl)imidazo[1,2-a]pyridine-3-carboxylate (200 mg, 613 μmol) in toluene (2.0 mL) was added 5-[5-[(1R,2S)-2-fluorocyclopropyl]-1,2,4-oxadiazol-3-yl]-2-methyl-aniline (172 mg, 735 μmol) and Al(CH₃)₃ (2 M, 766 μL). The mixture was stirred at 80° C. for 2 hours. The mixture was quenched by water (10 mL), then extracted with ethyl acetate (3×10 mL). The organic phase was concentrated in vacuo to give a residue. The residue was purified by prep-HPLC:column: Phenomenex luna C18 150*25 mm*10 um; mobile phase: [water(FA)-ACN]; gradient: 25%-55% B over 9 min to give compound N-[5-[5-[(1R,2S)-2-fluorocyclopropyl]-1,2,4-oxadiazol-3-yl]-2-methyl-phenyl]-7-(2-methylsulfonylethoxymethyl)imidazo[1,2-a]pyridine-3-carboxamide (I-454) (70 mg, 22.2% yield, 100% purity) as a white solid. ¹H NMR (400 MHz, DMSO-d₆) δ 10.02 (s, 1H), 9.41 (d, J=7.2 Hz, 1H), 8.57 (s, 1H), 8.02 (s, 1H), 7.78 (dd, J=1.6, 8.0 Hz, 1H), 7.73 (s, 1H), 7.48 (d, J=8.0 Hz, 1H), 7.13 (d, J=7.2 Hz, 1H), 5.40-5.17 (m, 1H), 4.67 (s, 2H), 3.89 (t, J=5.6 Hz, 2H), 3.48 (t, J=5.6 Hz, 2H), 3.13-3.04 (m, 1H), 3.03 (s, 3H), 2.35 (s, 3H), 2.01-1.86 (m, 1H), 1.65-1.52 (m, 1H). MS (ESI): m/z for C₂₄H₂₄FN₅O₅S [M+H]+ calcd. 514.2, [M+H]+ found. 514.2.

### Example 6—Preparation of 7-(2-hydroxyethoxymethyl)-N-[2-methyl-5-[5-(oxetan-3-yl)-1,2,4-oxadiazol-3-yl]phenyl]imidazo[1,2-a]pyridine-3-carboxamide (I-455)

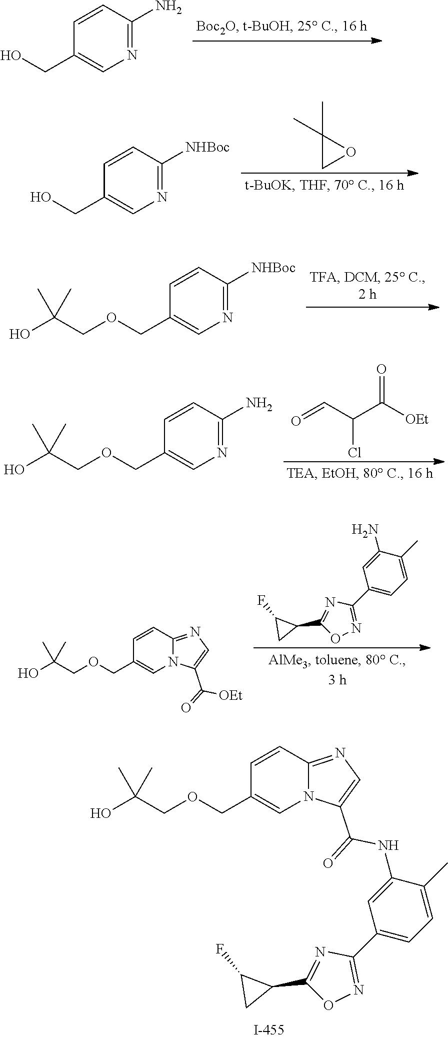

Step 1: To a solution of (6-amino-3-pyridyl) methanol (5.0 g, 40.3 mmol) in t-BuOH (50 mL) was added Boc₂O (13.2 g, 60.4 mmol, 13.9 mL, 1.5 eq). The mixture was stirred at 25° C. for 4 hours. After completion by LCMS and TLC, the mixture was concentrated in vacuo to give a residue. The residue was purified by column chromatography (SiO₂, petroleum ether/ethyl acetate=5/1 to 3/1). TLC (petroleum ether:ethyl acetate=3:1, Rf=0.5) to give tert-butyl N-[5-(hydroxymethyl)-2-pyridyl]carbamate (3.4 g, 30.5% yield) as a yellow solid. ¹H NMR (400 MHz, CDCl₃) δ 8.23 (d, J=1.6 Hz, 1H), 8.07-7.93 (m, 1H), 7.89-7.84 (m, 1H), 7.73 (br dd, J=2.4, 8.4 Hz, 2H), 4.67 (s, 2H), 1.55-1.53 (m, 9H). MS (ESI): m/z for C₁₁H₁₆N₂O₃ [M+H-56]+ calcd. 169.1, [M+H-56]+ found 169.1.

Step 2: To a solution of tert-butyl N-[5-(hydroxymethyl)-2-pyridyl]carbamate (2.9 g, 12.9 mmol) in THF (10 mL) was added t-BuOK in THF (1 M, 19.4 mL) and 2,2-dimethyloxirane (1.86 g, 25.9 mmol, 2.30 mL). The mixture was stirred at 70° C. for 16 hours. After completion by LCMS and TLC, the mixture was concentrated in vacuo to give a residue. The residue was purified by column chromatography (SiO₂, petroleum ether/ethyl acetate=5/1 to 3/1). TLC (petroleum ether:ethyl acetate=1:1, P1 Rf=0.49) to give tert-butyl N-[5-[(2-hydroxy-2-methyl-propoxy)methyl]-2-pyridyl]carbamate (3.5 g, 69.4% yield) as a yellow solid. ¹H NMR (400 MHz, CDCl₃) δ 8.22 (d, J=1.6 Hz, 1H), 8.01 (br d, J=8.4 Hz, 1H), 7.96-7.87 (m, 1H), 7.74-7.66 (m, 1H), 4.56-4.47 (m, 2H), 3.36-3.27 (m, 2H), 2.22 (br d, J=1.6 Hz, 1H), 1.56 (s, 9H), 1.25-1.20 (m, 6H).

Step 3: To a solution of tert-butyl N-[5-[(2-hydroxy-2-methyl-propoxy) methyl]-2-pyridyl] carbamate (900 mg, 3.04 mmol) in DCM (3.0 mL) was added TFA (346 mg, 3.04 mmol, 226 μL). The mixture was stirred at 25° C. for 1 hour. After completion on LCMS, the mixture was concentrated in vacuo to afford 1-[(6-amino-3-pyridyl)methoxy]-2-methyl-propan-2-ol (400 mg, crude) as a yellow solid and the crude product was used to the next step directly. MS (ESI): m/z for C₁₀H₁₆N₂O₂ [M+H]+ calcd. 197.1, [M+H]+ found 197.2.

Step 4: To a solution of 1-[(6-amino-3-pyridyl)methoxy]-2-methyl-propan-2-ol (400 mg, 2.04 mmol) in EtOH (2.0 mL) was added triethylamine (825 mg, 8.15 mmol, 1.13 mL) and ethyl 2-chloro-3-oxo-propanoate (460 mg, 3.06 mmol). The mixture was stirred at 80° C. for 16 hours. After completion by TLC, the mixture was concentrated in vacuo to give a residue. The residue was purified by column chromatography (SiO₂, Petroleum ether/ethyl acetate=5/1 to 3/1). TLC (petroleum ether:ethyl acetate=1:1, P1 Rf=0.5) to give compound ethyl 6-[(2-hydroxy-2-methyl-propoxy)methyl]imidazo[1,2-a]pyridine-3-carboxylate (300 mg, 50.4% yield) as a yellow solid. ¹H NMR (400 MHz, CDCl₃) δ 9.31 (s, 1H), 8.31 (s, 1H), 7.78 (d, J=9.2 Hz, 1H), 7.47 (d, J=9.2 Hz, 1H), 4.65 (s, 2H), 4.43 (q, J=7.2 Hz, 2H), 3.36 (s, 2H), 1.43 (t, J=7.2 Hz, 3H), 1.25 (s, 6H).

Step 5: To a solution of ethyl 6-[(2-hydroxy-2-methyl-propoxy)methyl]imidazo[1,2-a]pyridine-3-carboxylate (250 mg, 855 μmol) in toluene (1.0 mL) was added Al(CH₃)₃ (2 M, 1.07 mL) 5-[5-[(1R,2S)-2-fluorocyclopropyl]-1,2,4-oxadiazol-3-yl]-2-methyl-aniline (239 mg, 1.03 mmol). The mixture was stirred at 80° C. for 2 hours. After completion by LCMS, the mixture was quenched by water (10 mL), then extracted with ethyl acetate (3×10 mL). The combined organic phase was dried over Na₂SO₄, filtered and the filtrate was concentrated in vacuo to give a residue. The residue was purified by prep-HPLC: (column: Phenomenex luna C18 150*25 mm*10 um; mobile phase: [water (FA)-ACN]; gradient: 22%-52% B over 10 min) to give compound N-[5-[5-[(1R,2S)-2-fluorocyclopropyl]-1,2,4-oxadiazol-3-yl]-2-methyl-phenyl]-6-[(2-hydroxy-2-methyl-propoxy)methyl]imidazo[1,2-a]pyridine-3-carboxamide (I-455) (120 mg, 29.3% yield, 100% purity) as a white solid. ¹H NMR (400 MHz, DMSO-d6) δ 10.02 (s, 1H), 9.44 (s, 1H), 8.57 (s, 1H), 8.02 (d, J=1.6 Hz, 1H), 7.77 (d, J=9.6 Hz, 2H), 7.52-7.43 (m, 2H), 5.39-5.17 (m, 1H), 4.61 (s, 2H), 4.38 (s, 1H), 3.22 (s, 2H), 3.10-2.99 (m, 1H), 2.35 (s, 3H), 2.01-1.88 (m, 1H), 1.58 (qd, J=6.8, 13.2 Hz, 1H), 1.09 (s, 6H). MS (ESI): m/z for C₂₅H₂₆FN₅O₄[M+H]+ calcd. 480.2, [M+H]+ found 480.2.

### Example 7—Preparation of N-(5-(5-((1R,2S)-2-fluorocyclopropyl)-1,2,4-oxadiazol-3-yl)-2-methylphenyl)-6-(2-hydroxypropan-2-yl)imidazo[1,2-a]pyridine-3-carboxamide (I-457)

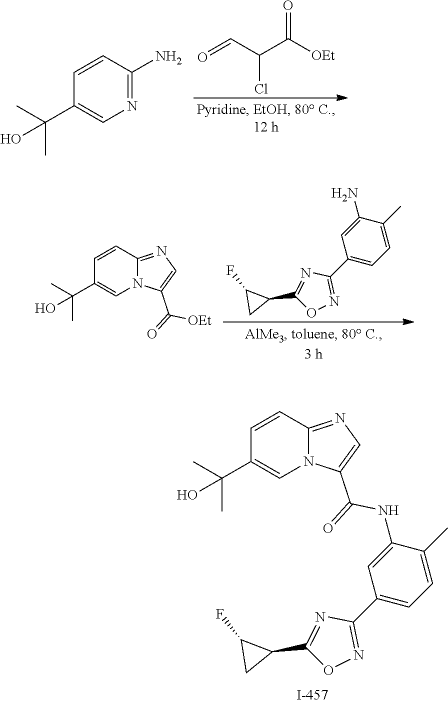

Step 1: To a solution of 2-(6-amino-3-pyridyl)propan-2-ol (3.0 g, 1 eq) in EtOH (30 mL) was added pyridine (1.72 g, 1.1 eq) and ethyl 2-chloro-3-oxo-propanoate (3.26 g, 1.1 eq). The mixture was stirred at 80° C. for 12 hours. The reaction mixture was concentrated under reduced pressure to remove EtOH. The residue was diluted with H₂O 200 mL and extracted with ethyl acetate (200 mL×3). The combined organic layers were washed with brine 600 mL (200 mL×3), dried over Na₂SO₄, filtered and concentrated under reduced pressure to give a residue. The residue was purified by column chromatography (SiO₂, petroleum ether/ethyl acetate=5/1 to 1/3) to give compound ethyl 6-(1-hydroxy-1-methyl-ethyl)imidazo[1,2-a]pyridine-3-carboxylate (2.75 g, 52% yield) as a yellow solid. ¹H NMR (400 MHz, DMSO-d₆) δ=9.35 (s, 1H), 8.25 (s, 1H), 7.76-7.72 (m, 1H), 7.69-7.63 (m, 1H), 5.43 (s, 1H), 4.39-4.31 (m, 2H), 1.50 (s, 6H), 1.34 (t, J=7.2 Hz, 3H). MS (ESI): m/z for C₁₃H₁₆N₂O₃ [M+H]+ calcd. 249.12, [M+H]+ found 248.8.

Step 2: To a solution of ethyl 6-(1-hydroxy-1-methyl-ethyl)imidazo[1,2-a]pyridine-3-carboxylate (400 mg, 1.0 eq) and 5-[5-[(1R,2S)-2-fluorocyclopropyl]-1,2,4-oxadiazol-3-yl]-2-methyl-aniline (376 mg, 1.0 eq) in toluene (5 mL) was added AlMe₃ (2 M, 2.5 eq) under nitrogen atmosphere. The mixture was stirred at 80° C. for 3 hours under N₂ atmosphere. The reaction mixture was quenched by addition NH₄Cl solution 4 mL at 0° C. and filtered, the filtrate was diluted with water 30 mL and extracted with ethyl acetate 150 mL (50 mL×3). The combined organic layers were washed with brine 90 mL (30 mL×3), dried over Na₂SO₄, filtered, and concentrated under reduced pressure to give a residue. The residue was purified by prep-HPLC (FA condition; column: Phenomenex luna C18 150*40 mm*15 um; mobile phase: [water(FA)-ACN]; gradient: 20%-50% B over 11 min) to give N-(5-(5-((1R,2S)-2-fluorocyclopropyl)-1,2,4-oxadiazol-3-yl)-2-methylphenyl)-6-(2-hydroxypropan-2-yl)imidazo[1,2-a]pyridine-3-carboxamide (I-457) (546.96 mg, 70.24% yield, FA) as a white solid. ¹H NMR (400 MHz, DMSO-d₆) δ=9.97 (s, 1H), 9.55 (d, J=0.8 Hz, 1H), 8.55 (s, 1H), 8.02 (d, J=1.6 Hz, 1H), 7.79-7.75 (m, 1H), 7.73-7.69 (m, 1H), 7.63-7.58 (m, 1H), 7.48 (d, J=8.0 Hz, 1H), 5.39-5.18 (m, 2H), 3.11-3.00 (m, 1H), 2.36 (s, 3H), 2.00-1.88 (m, 1H), 1.65-1.55 (m, 1H), 1.49 (s, 6H). MS (ESI): m/z for C₂₃H₂₂FN₅O₃[M+H]+ calcd. 436.17, [M+H]+ found 436.0.

### Example 8—Preparation of N-[5-[5-[(1R,2S)-2-fluorocyclopropyl]-1,2,4-oxadiazol-3-yl]-2-methyl-phenyl]-6-[(2-hydroxy-2-methylpropoxy) methyl]pyrazolo[1,5-a]pyridine-3-carboxamide (I-466)

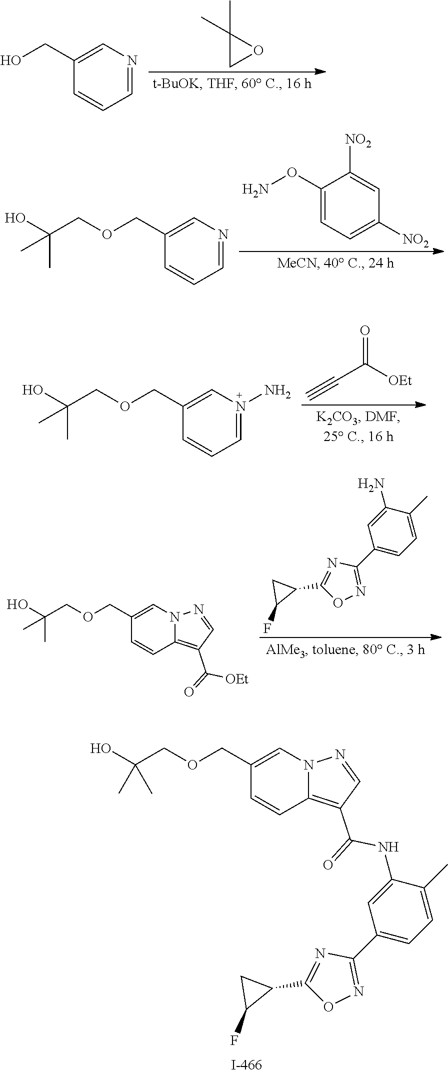

Step 1: To a mixture of 3-pyridylmethanol (15.0 g, 137 mmol, 1 eq) and 2,2-dimethyloxirane (11.9 g, 165 mmol, 1.2 eq) in THF (300 mL) was added t-BuOK (23.1 g, 206 mmol, 1.5 eq). The reaction mixture was stirred at 70° C. for 16 hrs. The reaction mixture was diluted with water (500 mL) and extracted with ethyl acetate (300 mL×3). The combined organic layers were washed with brine (500 mL×3). The organic layer was dried over anhydrous sodium sulfate and filtered. The filtrate was concentrated in vacuo. The crude product was purified by reversed-phase IPLC (0.1% FA condition) to give 2-methyl-1-(3-pyridylmethoxy)propan-2-ol (13.3 g, 73.4 mmol, 53.4% yield) as a yellow liquid. ¹H NMR (400 MHz, DMSO-d₆) δ: 8.55 (d, J=1.6 Hz, 1H), 8.52-8.47 (m, 1H), 7.75 (d, J=7.6 Hz, 1H), 7.44-7.32 (m, 1H), 4.55 (s, 2H), 4.39 (s, 1H), 3.21 (s, 2H), 1.09 (s, 6H).

Step 2: A mixture of 2-methyl-1-(3-pyridylmethoxy)propan-2-ol (6.76 g, 37.3 mmol, 1 eq) and O-(2,4-dinitrophenyl)hydroxylamine (7.43 g, 37.3 mmol, 1 eq) in ACN (80 mL) was stirred at 40° C. for 16 hrs. The reaction mixture was concentrated in vacuo to give 1-[(1-aminopyridin-1-ium-3-yl)methoxy]-2-methyl-propan-2-ol; 2,4-dinitrophenolate (13.0 g, 34.2 mmol, 91.6% yield) as a yellow oil. MS (ESI): m/z for C₁₀H₁₇N₂O₂+[M]+ calcd. 197.1, [M]+ found 197.3.

Step 3: A mixture of 1-[(1-aminopyridin-1-ium-3-yl)methoxy]-2-methyl-propan-2-ol; 2,4-dinitrophenolate (22.5 g, 59.16 mmol, 1 eq), ethyl prop-2-ynoate (5.80 g, 59.2 mmol, 1 eq) and K₂CO₃ (16.4 g, 118 mmol, 2 eq) in DMF (250 mL) was stirred at 25° C. for 16 hrs. The reaction mixture was diluted with water (500 mL) and extracted with ethyl acetate (200 mL×3). The combined organic layers were washed with brine (300 mL×3). The organic layer was dried over anhydrous sodium sulfate and filtered. The filtrate was concentrated in vacuo. The residue was purified by column chromatography (SiO2, petroleum ether:ethyl acetate=3:1) to give ethyl 6-[(2-hydroxy-2-methyl-propoxy)methyl]pyrazolo[1,5-a]pyridine-3-carboxylate (5.70 g, 19.5 mmol, 33.0% yield) as a yellow oil. ¹H NMR (400 MHz, DMSO-d₆) δ: 8.87 (s, 1H), 8.44 (s, 1H), 8.07 (d, J=9.2 Hz, 1H), 7.63-7.54 (m, 1H), 4.61 (s, 2H), 4.42 (s, 1H), 4.33-4.27 (m, 2H), 3.24 (s, 2H), 1.34 (t, J=7.2 Hz, 3H), 1.10 (s, 6H).

Step 4: To a solution of ethyl 6-[(2-hydroxy-2-methyl-propoxy)methyl]pyrazolo[1,5-a]pyridine-3-carboxylate (3 g, 10.3 mmol, 1 eq) and 5-[5-[(1R,2S)-2-fluorocyclopropyl]-1,2,4-oxadiazol-3-yl]-2-methyl-aniline (2.39 g, 10.3 mmol, 1 eq) in toluene (30 mL) was added AlMe₃ (2 M, 12.83 mL, 2.5 eq) and stirred at 80° C. for 3 hr under N₂. The reaction mixture was quenched by saturated aq. NH₄Cl (5 mL). The mixture was diluted with water (20 mL) and extracted with ethyl acetate (10 mL×3). The combined organic layers were washed with brine (30 mL×3). The organic layer was dried over anhydrous sodium sulfate and filtered. The filtrate was concentrated in vacuo. The residue was purified by silica gel chromatography (petroleum ether:ethyl acetate=3:1-1:2) to give N-[5-[5-[(1R,2S)-2-fluorocyclopropyl]-1,2,4-oxadiazol-3-yl]-2-methyl-phenyl]-6-[(2-hydroxy-2-methyl-propoxy)methyl]pyrazolo[1,5-a]pyridine-3-carboxamide (1.51 g, 3.15 mmol, 100% purity) as an off-white solid. ¹H NMR (400 MHz, DMSO-d₆) δ: 9.76 (s, 1H), 8.85 (s, 1H), 8.76 (s, 1H), 8.21 (d, J=9.2 Hz, 1H), 8.04 (d, J=1.6 Hz, 1H), 7.77-7.73 (m, 1H), 7.55-7.49 (m, 1H), 7.46 (d, J=8.0 Hz, 1H), 5.39-5.19 (m, 1H), 4.60 (s, 2H), 4.46 (s, 1H), 3.24 (s, 2H), 3.06 (s, 1H), 2.35 (s, 3H), 2.01-1.88 (m, 1H), 1.64-1.54 (m, 1H), 1.10 (s, 6H). MS (ESI): m/z for C₂₅H₂₆FN₅O₄[M+H]+ calcd. 480.2, [M+H]+ found 480.4.

### Example 9—Preparation of N-(5-(5-((1R,2S)-2-fluorocyclopropyl)-1,2,4-oxadiazol-3-yl)-2-methylphenyl)-7-(2-(2-hydroxy-2-methylpropoxy)ethyl)imidazo[1,2-a]pyridine-3-carboxamide (I-467)

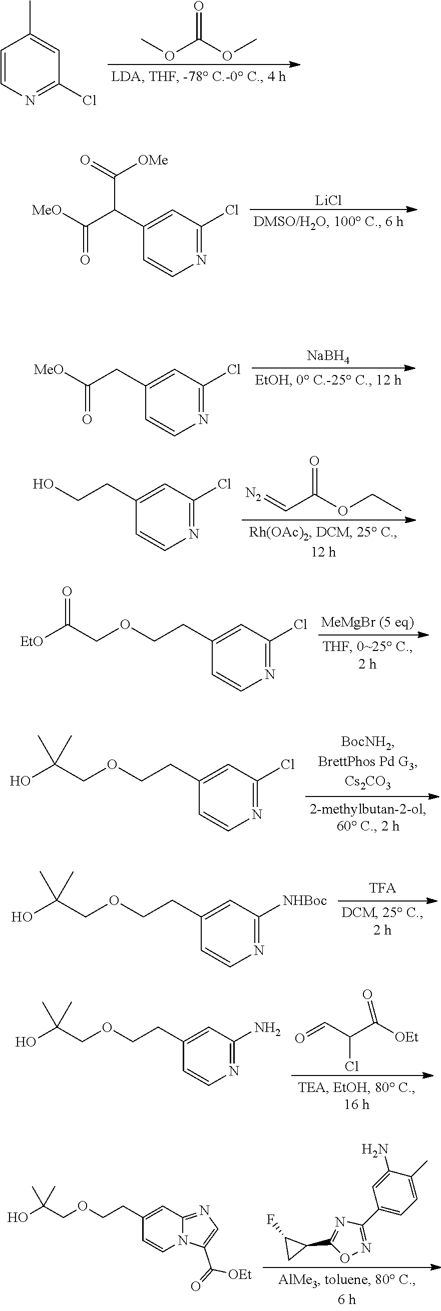

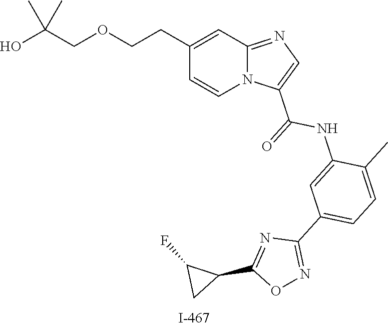 [SMILES: Cc1ccc(-c2noc([C@H]3C[C@@H]3F)n2)cc1NC(=O)c1cnc2cc(CCOCC(C)(C)O)ccn12]

Step 1: To a solution of 2-chloro-4-methyl-pyridine (58 g, 1.0 eq) in THF (1000 mL) was added LDA (2 M, 500 mL, 2.2 eq) dropwise slowly at nitrogen under −78° C. The mixture was stirred at −78° C. for 1 hour. To the mixture was added dimethyl carbonate (102.38 g, 2.5 eq) dropwise slowly. The mixture was stirred at −78° C. for 0.5 hour. The mixture was allowed to warm to 0° C. The mixture was stirred at 0° C. for 0.5 hour. The mixture was stirred at 25° C. for 12 hours. To the mixture was added 500 mL of NH₄Cl to quench the reaction. The aqueous layer was extracted with ethyl acetate (300 mL×2). The combined organic layers were washed with brine (500 mL), then dried over Na₂SO₄, filtered and evaporated to get residue. The residue was purified by column chromatography (SiO₂, petroleum ether/ethyl acetate=10/1 to 5/1) to give dimethyl 2-(2-chloro-4-pyridyl)propanedioate (75 g, 44% yield) as colorless oil. MS (ESI): m/z for C₁₀H₁₀ClNO₄ [M+H]+ calcd. 244.2, [M+H]+ found 244.2

Step 2: To a solution of dimethyl 2-(2-chloro-4-pyridyl)propanedioate (50 g, 1.0 eq) in DMSO (500 mL) and H₂O (50 mL) was added LiCl (52 g, 5.98 eq). The mixture was stirred at 100° C. for 6 hours. The reaction mixture was quenched by addition water 300 mL at 25° C., and extracted with ethyl acetate (40 mL×3). The combined organic layers were washed with brine (30 mL×3), dried over Na₂SO₄, filtered and concentrated under reduced pressure to give methyl 2-(2-chloro-4-pyridyl)acetate (39 g, crude) as red oil. MS (ESI): m/z for C₈H₈ClNO₂ [M+H]+ calcd. 186.2, [M+H]+ found 186.2

Step 3: To a solution of methyl 2-(2-chloro-4-pyridyl)acetate (45 g, 1.0 eq) in EtOH (450 mL) was added NaBH₄ (27.22 g, 2.97 eq) in several batches at 0° C. under nitrogen. The mixture was stirred at 25° C. for 12 hours. To the mixture was added 1000 mL of water at 0° C. The mixture was stirred at 25° C. for 10 minutes. The mixture was extracted with dichloromethane (300 mL×3) and the organic layer was washed with brine 100 ml. The mixture was dried over Na₂SO₄, filtered and concentrated to give a residue. The residue was purified by column chromatography (SiO₂, petroleum ether/ethyl acetate=5/1 to 1/1) to give 2-(2-chloro-4-pyridyl)ethanol (27 g, 66% yield) as yellow oil. MS (ESI): m/z for C₇H₈ClNO [M+H]+ calcd. 158.0, [M+H]+ found 158.0. ¹H NMR (400 MHz, CDCl₃) δ ppm 8.25 (d, J=5.2 Hz, 1H), 7.25 (s, 1H), 7.13 (dd, J=5.2, 0.8 Hz, 1H), 3.93 (t, J=6.4 Hz, 2H), 2.87 (t, J=6.4 Hz, 2H).

Step 4: To a solution of 2-(2-chloro-4-pyridyl)ethanol (10 g, 1.0 eq) in DCM (100 mL) was added Rh(OAc)₂ (701 mg, 0.05 eq) under nitrogen. To the mixture was added ethyl 2-diazoacetate (21.72 g, 20 mL, 3.0 eq) dropwise slowly (over 2 hours, drop speed with 10 mL/h). The mixture was stirred at 25° C. for 12 hours. The mixture was concentrated to give a residue. The residue was purified by column chromatography (SiO₂, petroleum ether/ethyl acetate=10/1 to 3/1) to give ethyl 2-[2-(2-chloro-4-pyridyl)ethoxy]acetate (6.5 g, 26 mmol, 42% yield) as yellow oil. MS (ESI): m/z for: C₁₁H₁₄ClNO₃ [M+H]+ calcd. 244.1, [M+H]+ found 244.1. ¹H NMR (400 MHz, CHLOROFORM-d) δ ppm 8.31-8.30 (d, J=5.2 Hz, 1H), 7.28 (s, 1H), 7.17 (dd, J=5.2, 0.8 Hz, 1H), 4.23 (q, J=7.2 Hz, 2H), 4.08 (s, 2H), 3.81 (t, J=6.4 Hz, 2H), 2.95 (t, J=6.4 Hz, 2H), 1.36-1.23 (m, 3H).

Step 5: To a solution of ethyl 2-[2-(2-chloro-4-pyridyl)ethoxy]acetate (4.5 g, 1.0 eq) in THF (60 mL) was added bromo(methyl)magnesium (3 M, 5.0 eq) dropwise at 0° C. under nitrogen. The mixture was stirred at 25° C. for 2 hours. The reaction mixture was quenched by addition NH₄Cl 10 mL at 25° C., and extracted with ethyl acetate (15 mL×3). The combined organic layers were washed with brine 10 mL, dried over Na₂SO₄, filtered and concentrated under reduced pressure to give a residue. The residue was purified by column chromatography (SiO2, Petroleum ether/ethyl acetate=10/1 to 1/1) to give 1-[2-(2-chloro-4-pyridyl)ethoxy]-2-methyl-propan-2-ol (2.4 g, 51% yield) as yellow oil. MS (ESI): m/z for: C₁₁H₁₆ClNO₂ [M+H]+ calcd. 230.1, [M+H]+ found 230.1. ¹H NMR (400 MHz, CHLOROFORM-d) δ ppm 8.30 (d, J=4.8 Hz, 1H), 7.24 (s, 1H), 7.12 (dd, J=5.2, 1.2 Hz, 1H), 3.74 (t, J=6.4 Hz, 2H), 3.28 (s, 2H), 2.91 (t, J=6.4 Hz, 2H), 1.19 (s, 6H).

Step 6: To a solution of 1-[2-(2-chloro-4-pyridyl)ethoxy]-2-methyl-propan-2-ol (2.7 g, 1.0 eq) and tert-butyl carbamate (4.13 g, 3.0 eq) in 2-methylbutan-2-ol (40 mL) was added BrettPhos Pd G₃ (1.07 g, 0.1 eq) and Cs₂CO₃ (11.49 g, 3.0 eq) under nitrogen. The mixture was stirred at 65° C. for 2 hours. The mixture was filtered and concentrated to give a residue. The residue was purified by column chromatography (SiO₂, petroleum ether/ethyl acetate=10/1 to 1/1) to give tert-butyl N-[4-[2-(2-hydroxy-2-methyl-propoxy)ethyl]-2-pyridyl]carbamate (3 g, 76% yield) as yellow solid. MS (ESI): m/z for: C₁₆H₂₆N₂O₄ [M+H]+ calcd. 311.2, [M+H]+ found 311.4. ¹H NMR (400 MHz, CHLOROFORM-d) δ ppm 8.55-8.42 (m, 1H), 8.22-8.15 (m, 1H), 7.93 (s, 1H), 6.85-6.83 (dd, J=5.2, 1.2 Hz, 1H), 3.74 (t, J=6.4 Hz, 2H), 3.28 (s, 2H), 2.90 (t, J=6.4 Hz, 2H), 1.54 (s, 9H), 1.19 (s, 6H).

Step 7: To a solution of tert-butyl N-[4-[2-(2-hydroxy-2-methyl-propoxy)ethyl]-2-pyridyl]carbamate (4 g, 1.0 eq) in DCM (40 mL) was added TFA (20.47 g, 13.33 mL, 13.93 eq). The mixture was stirred at 25° C. for 2 hours. The mixture was concentrated to give 1-[2-(2-amino-4-pyridyl)ethoxy]-2-methyl-propan-2-ol (2.7 g, crude) as yellow oil was obtained, which was used next step without further purification. MS (ESI): m/z for: C₁₁H₁₈N₂O₂ [M+H]+ calcd. 211.1, [M+H]+ found 211.2.

Step 8: To a solution of 1-[2-(2-amino-4-pyridyl)ethoxy]-2-methyl-propan-2-ol (2.7 g, 1.0 eq) and ethyl 2-chloro3-oxo-propanoate (2.32 g, 1.2 eq) in EtOH (40 mL) was added triethylamine (6.50 g, 8.94 mL, 5 eq). The mixture was stirred at 80° C. for 6 hours. The mixture was concentrated to give a residue. The residue was purified by column chromatography (SiO2, petroleum ether/ethyl acetate=1/1 to 1/3) to give ethyl 7-[2-(2-hydroxy-2-methyl-propoxy)ethyl]imidazo[1,2-a]pyridine-3-carboxylate (3.4 g, 86% yield) as red oil. MS (ESI): m/z for: C₁₆H₂₂N₂O₄ [M+H]+ calcd. 307.2, [M+H]+ found 307.3. ¹H NMR (400 MHz, chloroform-d) δ ppm 9.21 (d, J=7.2 Hz, 1H), 8.27 (s, 1H), 7.60 (s, 1H), 6.98 (dd, J=7.2, 1.6 Hz, 1H), 4.42 (q, J=7.2 Hz, 2H), 3.80 (t, J=6.4 Hz, 2H), 3.30 (s, 2H), 3.02 (t, J=6.4 Hz, 2H), 1.42 (t, J=7.2 Hz, 3H), 1.19 (s, 6H).

Step 9: To a solution of ethyl 7-[2-(2-hydroxy-2-methyl-propoxy)ethyl]imidazo[1,2-a]pyridine-3-carboxylate (3.4 g, 1.0 eq) and 5-[5-[(1R,2S)-2-fluorocyclopropyl]-1,2,4-oxadiazol-3-yl]-2-methyl-aniline (3.11 g, 1.2 eq) in toluene (40 mL) was added Al(CH₃)₃ (2 M, 13.87 mL, 2.5 eq) under nitrogen. The mixture was stirred at 80° C. for 6 hours. The mixture was cooled to 25° C. The mixture was poured into 100 mL of ice/water and extracted with DCM (100 mL×3). The combined organic layers were washed with brine 50 mL, dried over Na₂SO₄, filtered and concentrated under reduced pressure to give a residue. The residue was purified by column chromatography (SiO2, petroleum ether/ethyl acetate=1/1 to 0/1 to ethyl acetate:ethanol=20:1) to give compound N-(5-(5-((1R,2S)-2-fluorocyclopropyl)-1,2,4-oxadiazol-3-yl)-2-methylphenyl)-7-(2-(2-hydroxy-2-methylpropoxy)ethyl)imidazo[1,2-a]pyridine-3-carboxamide (2.95 g, 53% yield) as an off-white solid. MS (ESI): m/z for: C₂₆H₂₈FN₅O₄[M+H]+ calcd. 494.2, [M+H]+ found 494.4. ¹H NMR (400 MHz, chloroform-d) δ ppm 9.37 (d, J=7.2 Hz, 1H), 8.42 (s, 1H), 8.33 (d, J=1.6 Hz, 1H), 8.26 (s, 1H), 7.76 (dd, J=8.0, 1.6 Hz, 1H), 7.45 (s, 1H), 7.29 (d, J=8.0 Hz, 1H), 6.89 (dd, J=7.2, 1.6 Hz, 1H), 5.15-4.90 (m, 1H), 3.74 (t, J=6.4 Hz, 2H), 3.26 (s, 2H), 2.94 (t, J=6.4 Hz, 2H), 2.74-2.62 (m, 1H), 2.62-2.52 (m, 1H), 2.34 (s, 3H), 1.87-1.73 (m, 1H), 1.57 (dd, J=12.0, 6.4 Hz, 1H) 1.24 (s, 6H).

### Example 10—Preparation of N-[5-[5-[(1R,2S)-2-fluorocyclopropyl]-1,2,4-oxadiazol-3-yl]-2-methyl-phenyl]-7-[[(2R*)-2-hydroxypropoxy]methyl]imidazo[1,2-a]pyridine-3-carboxamide (I-468) and N-[5-[5-[(1R,2S)-2-fluorocyclopropyl]-1,2,4-oxadiazol-3-yl]-2-methyl-phenyl]-7-[[(2S*)-2-hydroxypropoxy]methyl]imidazo[1,2-a]pyridine-3-carboxamide (I-469)

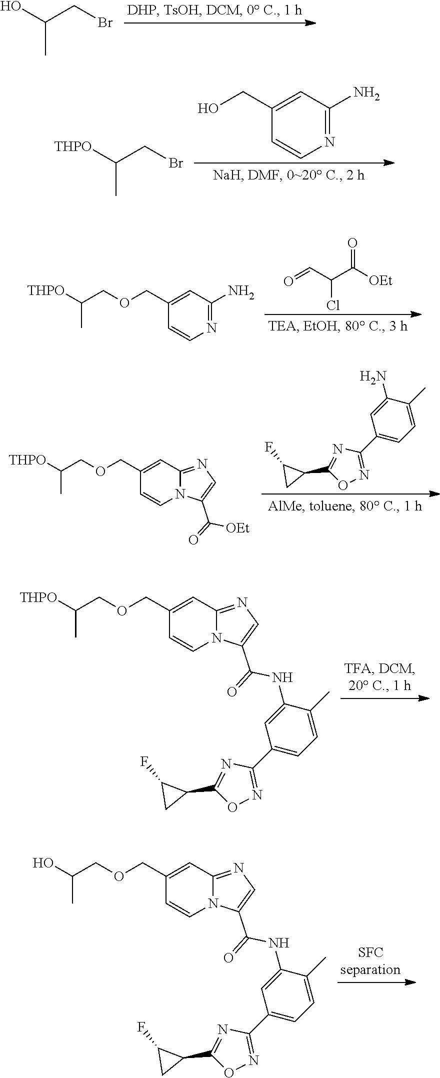

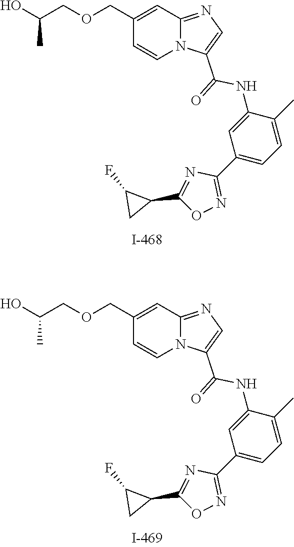 [SMILES: Cc1ccc(-c2noc([C@H]3C[C@@H]3F)n2)cc1NC(=O)c1cnc2cc(COC[C@@H](C)O)ccn12 ; Cc1ccc(-c2noc([C@H]3C[C@@H]3F)n2)cc1NC(=O)c1cnc2cc(COC[C@H](C)O)ccn12]

Step 1: To a solution of 1-bromopropan-2-ol (10 g, 71.95 mmol, 1 eq) in DCM (120 mL) was added TsOH (619.47 mg, 3.60 mmol, 0.05 eq) and DHP (7.26 g, 86.34 mmol, 7.89 mL, 1.2 eq). The mixture was stirred at 0° C. for 1 hour. The mixture was concentrated in vacuo to give a residue. The residue was purified by column chromatography (SiO₂, petroleum ether:ethyl acetate=30:1) to give 2-((1-bromopropan-2-yl)oxy)tetrahydro-2H-pyran (9.5 g, 42.58 mmol, 95.00% yield, 100% purity) as a colorless oil. ¹H NMR (400 MHz, CHLOROFORM-d) δ=4.76-4.64 (m, 1H), 4.20-3.86 (m, 2H), 3.68-3.44 (m, 2H), 3.41-3.35 (m, 1H), 1.87-1.79 (m, 1H), 1.77-1.68 (2H), 1.65-1.58 (m, 1H), 1.54-1.48 (m, 2H), 1.39-1.18 (m, 3H).

Step 2: To a solution of (2-amino-4-pyridyl)methanol (1.67 g, 13.45 mmol, 1 eq) in DMF (35 mL) was added NaH (806.71 mg, 20.17 mmol, 60% purity, 1.5 eq) at 0° C., the mixture was stirred for 5 minutes, Then 2-(2-bromo-1-methyl-ethoxy)tetrahydropyran (6 g, 26.89 mmol, 2 eq) was added and the mixture was stirred at 20° C. for 2 hours. The reaction mixture was quenched by addition water 30 mL at 0° C., and then diluted with water 200 mL and extracted with ethyl acetate 900 mL (300 mL×3). The combined organic layers were washed with brine 600 mL (100 mL×6), dried over Na₂SO₄, filtered and concentrated under reduced pressure to give 4-((2-((tetrahydro-2H-pyran-2-yl)oxy)propoxy)methyl)pyridin-2-amine (3.4 g, crude) as a yellow oil. MS (ESI): m/z for C₁₄H₂₂N₂O₃ [M+H]+ calcd. 266.9, [M+H]+ found. 266.16.

Step 3: To a solution of 4-(2-tetrahydropyran-2-yloxypropoxymethyl)pyridin-2-amine (3.4 g, 12.77 mmol, 1 eq) and ethyl 2-chloro-3-oxo-propanoate (1.92 g, 12.77 mmol, 1 eq) in EtOH (30 mL) was added triethylamine (2.58 g, 25.53 mmol, 3.55 mL, 2 eq). The mixture was stirred at 80° C. for 3 hours. The mixture was concentrated in vacuo. The residue was diluted with ethyl acetate (200 mL) and washed with brine (100 mL×3). The organic layer was dried over Na₂SO₄, filtered and concentrated in vacuo. The residue was purified by column chromatography (SiO₂, petroleum ether/ethyl acetate=5/1 to 1/1) to give ethyl 7-((2-((tetrahydro-2H-pyran-2-yl)oxy)propoxy)methyl)imidazo[1,2-a]pyridine-3-carboxylate (445 mg, 1.15 mmol, 9.03% yield, 93.90% purity) as a yellow oil. ¹H NMR (400 MHz, DMSO-d₆) δ=9.18 (d, J=6.8 Hz, 1H), 8.27 (s, 1H), 7.74-7.68 (m, 1H), 7.23-7.16 (m, 1H), 4.80-4.70 (m, 1H), 4.69-4.59 (m, 2H), 4.40-4.32 (m, 2H), 3.95-3.88 (m, 1H), 3.87-3.74 (m, 1H), 3.50-3.35 (m, 3H), 1.75-1.57 (m, 2H), 1.50-1.39 (m, 4H), 1.34 (t, J=7.0 Hz, 3H), 1.17-1.09 (m, 3H). MS (ESI): m/z for C₁₉H₂₆N₂O₅ [M+H]+ calcd. 363.0, [MH]+ found 362.18.

Step 4: To a solution of ethyl 7-(2-tetrahydropyran-2-yloxypropoxymethyl)imidazo[1,2-a]pyridine-3-carboxylate (390 mg, 1.08 mmol, 1 eq) and 5-[5-[(1R,2S)-2-fluorocyclopropyl]-1,2,4-oxadiazol-3-yl]-2-methyl-aniline (301.19 mg, 1.29 mmol, 1.2 eq) in the toluene (4 mL) was added AlMe₃ (2 M, 1.35 mL, 2.5 eq). The mixture was stirred at 80° C. for 1 hour under N₂ atmosphere. The reaction mixture was quenched by addition NH₄Cl solution 5 mL at 0° C. and filtered, the filtrate was diluted with water 80 mL and extracted with ethyl acetate 300 mL (100 mL×3). The combined organic layers were washed with brine 300 mL (100 mL×3), dried over [Na₂SO₄], filtered and concentrated under reduced pressure to give N-(5-(5-((1R,2S)-2-fluorocyclopropyl)-1,2,4-oxadiazol-3-yl)-2-methylphenyl)-7-((2-((tetrahydro-2H-pyran-2-yl)oxy)propoxy)methyl)imidazo[1,2-a]pyridine-3-carboxamide (700 mg, crude) as a yellow oil. MS (ESI): m/z for C₂₉H₃₂FN₅O₅[M+H]+ calcd. 550.1, [MH]+ found 549.24.

Step 5: To a solution of N-[5-[5-[(1R,2S)-2-fluorocyclopropyl]-1,2,4-oxadiazol-3-yl]-2-methyl-phenyl]-7-(2-tetrahydropyran-2-yloxypropoxymethyl)imidazo[1,2-a]pyridine-3-carboxamide (650 mg, 1.18 mmol, 1 eq) in DCM (8 mL) was added TFA (2 mL). The mixture was stirred at 20° C. for 1 hour. The mixture was concentrated in vacuo. The residue was purified by prep-HPLC (FA condition; column: Phenomenex luna C18 150*40 mm*15 um; mobile phase: [water(FA)-ACN]; gradient:20%-50% B over 11 min) to give N-(5-(5-((1R,2S)-2-fluorocyclopropyl)-1,2,4-oxadiazol-3-yl)-2-methylphenyl)-7-((2-hydroxypropoxy)methyl)imidazo[1,2-a]pyridine-3-carboxamide (250 mg, 532.30 μmol, 45.01% yield, 99.11% purity) as a white solid. ¹H NMR (400 MHz, DMSO-d₆) δ=10.02 (s, 1H), 9.41 (d, J=7.2 Hz, 1H), 8.58 (s, 1H), 8.03 (s, 1H), 7.78 (d, J=8.4 Hz, 1H), 7.73 (s, 1H), 7.49 (d, J=8.0 Hz, 1H), 7.15 (d, J=7.2 Hz, 1H), 5.40-5.20 (m, 1H), 4.66-4.61 (m, 3H), 3.87-3.80 (m, 1H), 3.36-3.30 (m, 2H), 3.12-3.02 (m, 1H), 2.36 (s, 3H), 2.00-1.90 (m, 1H), 1.64-5.54 (m, 1H), 1.09 (d, J=6.4 Hz, 3H). MS (ESI): m/z for C₂₄H₂₄FN₅O₄[M+H]⁺ calcd. 466.0, [MH]⁺ found 465.18.

Step 6: The residue was separated by SFC (condition:column: DAICEL CHIRALPAK AD (250 mm*30 mm, 10 um); mobile phase: [CO2-ACN/EtOH (0.1% NH3H2O)]; B %: 70%, isocratic elution mode) to give compound N-[5-[5-[(1R,2S)-2-fluorocyclopropyl]-1,2,4-oxadiazol-3-yl]-2-methyl-phenyl]-7-[[(2R*)-2-hydroxypropoxy]methyl]imidazo[1,2-a]pyridine-3-carboxamide (29.31 mg, 62.97 μmol, 29.31% yield) as white solid and compound N-[5-[5-[(1R,2S)-2-fluorocyclopropyl]-1,2,4-oxadiazol-3-yl]-2-methyl-phenyl]-7-[[(2S*)-2-hydroxypropoxy]methyl]imidazo[1,2-a]pyridine-3-carboxamide (59.72 mg, 116.75 μmol, 54.35% yield, FA) as white solid.

I-469: ¹H NMR (400 MHz, DMSO-d₆) δ=10.00 (s, 1H), 9.41-9.37 (m, 1H), 8.56 (s, 1H), 8.02 (d, J=1.6 Hz, 1H), 7.80-7.75 (m, 1H), 7.72 (s, 1H), 7.48 (d, J=8.0 Hz, 1H), 7.15-7.11 (m, 1H), 5.38-5.18 (m, 1H), 4.72-4.67 (m, 1H), 4.63 (s, 2H), 3.88-3.77 (m, 1H), 3.41-3.33 (m, 2H), 3.10-2.99 (m, 1H), 2.35 (s, 3H), 2.01-1.88 (m, 1H), 1.63-1.54 (m, 1H), 1.08 (d, J=6.4 Hz, 3H). MS (ESI): m/z for C₂₄H₂₄FN₅O₄[M+H]+ calcd. 466.4, [M+H]+ found 464.4.

I-468: ¹H NMR (400 MHz, DMSO-d₆) δ=10.00 (s, 1H), 9.39 (d, J=7.2 Hz, 1H), 8.56 (s, 1H), 8.02 (d, J=1.6 Hz, 1H), 7.80-7.75 (m, 1H), 7.72 (s, 1H), 7.48 (d, J=8.0 Hz, 1H), 7.15-7.11 (m, 1H), 5.39-5.18 (m, 1H), 4.70 (d, J=4.4 Hz, 1H), 4.63 (s, 2H), 3.88-3.79 (m, 1H), 3.44-3.33 (m, 2H), 3.12-3.00 (m, 1H), 2.35 (s, 3H), 2.01-1.87 (m, 1H), 1.63-1.54 (m, 1H), 1.09-1.05 (m, 3H). MS (ESI): m/z for C₂₄H₂₄FN₅O₄[M+H]+ calcd. 466.2, [M+H]+ found 466.4.

### Example 11—Preparation of (6R*)—N-[5-[5-[(1R,2S)-2-fluorocyclopropyl]-1,2,4-oxadiazol-3-yl]-2-methyl-phenyl]-6-methyl-5-oxa-1,10-diazatricyclo[7.3.0.03′⁷]dodeca-2,7,9,11-tetraene-12-carboxamide (I-470) and (6S*)—N-[5-[5-[(1R,2S)-2-fluorocyclopropyl]-1,2,4-oxadiazol-3-yl]-2-methyl-phenyl]-6-methyl-5-oxa-1,10-diazatricyclo[7.3.0.03′⁷]dodeca-2,7,9,11-tetraene-12-carboxamide (I-471)

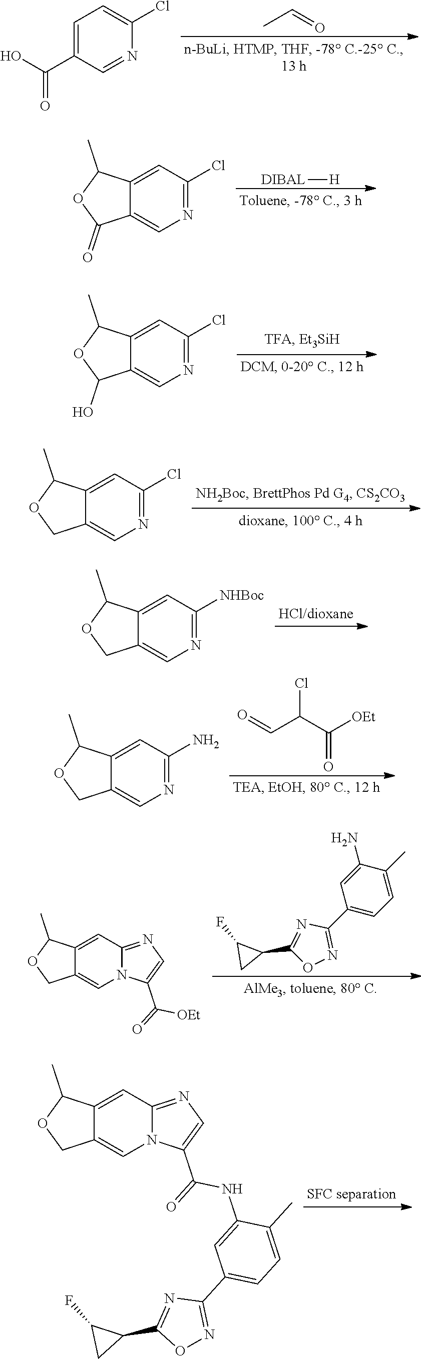

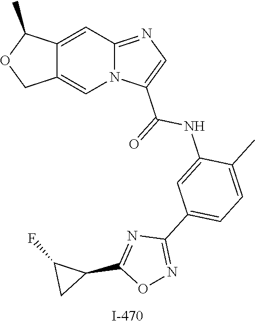 [SMILES: Cc1ccc(-c2noc([C@H]3C[C@@H]3F)n2)cc1NC(=O)c1cnc2cc3c(cn12)CO[C@H]3C]

Step 1: To a solution of HTMP (13.99 g, 2.4 eq) in THF (120 mL) was added n-BuLi (2.5 M, 36.30 mL, 2.2 eq) at −78° C. under nitrogen, the mixture was stirred at −70° C. for 1 hour, 6-chloropyridine-3-carboxylic acid (6.5 g, 1.0 eq) in THF (60 mL) was added at −70° C. The resulting mixture was stirred for 1 hour at −70° C., acetaldehyde (5 M, 8.25 mL, 1.0 eq) was added and the mixture was stirred at −70° C. for 1 hour. The reaction was quenched by NH₄Cl sat. (50 mL) and warmed up to 25° C. After acidified to pH=2 with HCl (2 M), the resulting mixture was heated to 70° C. for 10 hours. The reaction mixture was extracted with ethyl acetate (500 mL), the organic layer was washed with brine (300 mL×2), dried over Na₂SO₄, filtered and concentrated in vacuum to give residue. The residue was purified by column chromatography (SiO₂, petroleum ether/ethyl acetate=10/1 to 3/1) to give 6-chloro-1-methyl-1H-furo[3,4-c]pyridin-3-one (3 g, 40% yield) as yellow solid. ¹H NMR (400 MHz, DMSO-d₆) δ 8.92 (s, 1H), 8.00 (s, 1H), 5.76 (q, J=6.8 Hz, 1H), 1.59 (d, J=6.8 Hz, 3H). MS (ESI): m/z for C₁₃H₁₆N₂O₃ [M+H]+ calcd. 184.1, [M+H]+ found 184.1.

Step 2: To a solution of 6-chloro-1-methyl-1H-furo[3,4-c]pyridin-3-one (1 g, 1 eq) in toluene (20 mL) was added DIBAL-H (1 M, 10.89 mL, 2 eq) slowly at −78° C. under N₂, the reaction mixture was stirred at −78° C. for 3 hours under nitrogen. The reaction mixture was quenched with NH₄Cl sat. (10 mL), extracted with ethyl acetate (50 mL×2), the organic layers were washed with brine (50 mL×3), dried over Na₂SO₄, filtered and concentrated in vacuum to give residue. The residue was purified by column chromatography (SiO₂, petroleum ether/ethyl acetate=10/1 to 3/1) to give 6-chloro-1-methyl-1,3-dihydrofuro[3,4-c]pyridin-3-ol (600 mg, 49% yield) as colorless oil. ¹H NMR (400 MHz, DMSO-d₆) δ 8.45-8.38 (m, 1H), 7.58 (d, J=4.0 Hz, 1H), 7.11-6.90 (m, 1H), 6.42-6.30 (m, 1H), 5.36-5.09 (m, 1H), 1.49-1.38 (m, 3H). MS (ESI): m/z for C₂₂H₁₇N₆O₂F [M+H]+ calcd. 185.8, [M+H]+ found 185.8.

Step 3: To a solution of 6-chloro-1-methyl-1,3-dihydrofuro[3,4-c]pyridin-3-ol (500 mg, 1.0 eq) in DCM (10 mL) was added TFA (3.07 g, 10 eq) at 0° C. under nitrogen, the mixture was stirred at 0° C. for 0.5 hours, Then Et₃SiH (1.57 g, 13.47 mmol, 2.15 mL, 5 eq) was added the mixture at 0° C. under nitrogen, the reaction mixture was stirred at 25° C. for 12 hours under nitrogen. The reaction mixture quenched with saturated NaHCO₃ (30 mL), extracted with DCM (50 mL×2), the combined organic layers were washed with brine (20 mL×2), dried over Na₂SO₄, filtered and concentrated in vacuum to give residue. The residue was purified by column chromatography (SiO₂, petroleum ether/ethyl acetate=10/1 to 5/1) to give 6-chloro-1-methyl-1,3-dihydrofuro[3,4-c]pyridine (400 mg, 86% yield) as colorless oil. ¹H NMR (400 MHz, CDCl₃-d) δ 8.28 (s, 1H), 7.15 (s, 1H), 5.33-5.24 (m, 1H), 5.19-5.00 (m, 2H), 1.51 (d, J=6.4 Hz, 3H). MS (ESI): m/z for C₂₂H₁₇N₆O₂F [M+H]+ calcd. 169.8, [M+H]+ found 169.8.

Step 4: To a solution of 6-chloro-1-methyl-1,3-dihydrofuro[3,4-c]pyridine (300 mg, 1.0 eq), Cs₂CO₃ (1.73 g, 3.0 eq) and NH₂Boc (621 mg, 3.0 eq) in dioxane (20 mL) was added BrettPhos (Pd, G₄) (163 mg, 0.1 eq), the reaction was stirred at 100° C. for 4 hours under N₂. The reaction mixture was diluted with ethyl acetate (60 mL), washed with brine (50 mL×3), the organic layer was dried over Na₂SO₄, filtered and concentrated in vacuum to give residue. The residue was purified by column chromatography (SiO₂, petroleum ether/ethyl acetate=10/1 to 3/1) to give tert-butyl N-(1-methyl-1,3-dihydrofuro[3,4-c]pyridin-6-yl)carbamate (300 mg, 58% yield) as yellow solid. ¹H NMR (400 MHz, CDCl₃-d) δ 8.11 (s, 1H), 7.92 (br s, 1H), 7.83 (s, 1H), 5.26 (q, J=6.4 Hz, 1H), 5.14-5.07 (m, 1H), 5.03-4.97 (m, 1H), 1.56-1.51 (m, 12H). MS (ESI): m/z for C₂₂H₁₇N₆O₂F [M-56+H]+ calcd. 195.2, [M-56+H]+ found. 195.2.

Step 5: The mixture of tert-butyl N-(1-methyl-1,3-dihydrofuro[3,4-c]pyridin-6-yl)carbamate (300 mg, 1.0 eq) in HCl/dioxane (5 mL) was stirred at 25° C. for 16 hours. The reaction was concentrated in vacuum to give 1-methyl-1,3-dihydrofuro[3,4-c]pyridin-6-amine (220 mg, 76% yield, HCl) as yellow solid. MS (ESI): m/z for C₂₂H₁₇N₆O₂F [M+H]+ calcd. 151.2, [M+H]+ found 151.2.

Step 6: To a solution of 1-methyl-1,3-dihydrofuro[3,4-c]pyridin-6-amine (430 mg, 1.0 eq, HCl) and ethyl 2-chloro-3-oxo-propanoate (867 mg, 2.5 eq) in EtOH (15 mL) was added triethylamine (699 mg, 3.0 eq), the reaction mixture was stirred at 80° C. for 12 hours. The reaction mixture was diluted with ethyl acetate (80 mL), washed with brine (40 mL×3), the organic layer was dried over Na₂SO₄, filtered and concentrated in vacuum to give residue. The residue was purified by column chromatography (SiO₂, petroleum ether/ethyl acetate=5/1 to 1/1) to give ethyl 6-methyl-5-oxa-1,10-diazatricyclo[7.3.0.0^{3,7}]dodeca-2,7,9,11-tetraene-12-carboxylate (420 mg, 74% yield) as yellow solid. ¹H NMR (400 MHz, DMSO-d₆) δ 9.20 (d, J=0.8 Hz, 1H), 8.26 (s, 1H), 7.72 (s, 1H), 5.24 (q, J=6.4 Hz, 1H), 5.12 (d, J=12.8 Hz, 1H), 5.03-4.93 (m, 1H), 4.35 (q, J=7.2 Hz, 2H), 1.49 (d, J=6.4 Hz, 3H), 1.34 (t, J=7.2 Hz, 3H). MS (ESI): m/z for C₂₂H₁₇N₆O₂F [M+H]+ calcd. 247.2, [M+H]+ found 247.2.

Step 7: To a solution of ethyl 6-methyl-5-oxa-1,10-diazatricyclo[7.3.0.0^{3,7}]dodeca-2,7,9,11-tetraene-12-carboxylate (400 mg, 1.0 eq) and 5-[5-[(1R,2S)-2-fluorocyclopropyl]-1,2,4-oxadiazol-3-yl]-2-methyl-aniline (379 mg, 1.0 eq) in toluene (15 mL) was added AlMe₃ (2 M, 2.03 mL, 2.5 eq) under nitrogen at 0° C., the reaction mixture was stirred at 80° C. for 3 hours. The reaction was quenched with NH₄Cl sat. (10 mL), extracted with ethyl acetate (50 mL×3), the combined organic layers were washed with brine (20 mL×2), dried over Na₂SO₄, filtered and concentrated in vacuum. The residue was purified by column chromatography (SiO₂, petroleum ether/ethyl acetate=5/1 to 0/1) to give N-[5-[5-[(1R,2S)-2-fluorocyclopropyl]-1,2,4-oxadiazol-3-yl]-2-methyl-phenyl]-6-methyl-5-oxa-1,10 diazatricyclo [7.3.0.0^{3,7}]dodeca-2,7,9,11-tetraene-12-carboxamide (500 mg, 69% yield) as yellow solid. ¹H NMR (400 MHz, DMSO-d₆) δ 9.99 (s, 1H), 9.42 (d, J=0.8 Hz, 1H), 8.55 (s, 1H), 8.04 (d, J=1.6 Hz, 1H), 7.77 (dd, J=1.6, 8.0 Hz, 1H), 7.68 (s, 1H), 7.48 (d, J=8.0 Hz, 1H), 5.42-5.17 (m, 2H), 5.10 (d, J=12.4 Hz, 1H), 4.96 (d, J=12.8 Hz, 1H), 3.12-2.99 (m, 1H), 2.35 (s, 3H), 2.01-1.87 (m, 1H), 1.64-1.54 (m, 1H), 1.50 (d, J=6.4 Hz, 3H). MS (ESI): m/z for C₂₂H₁₇N₆O₂F [M+H]+ calcd. 433.9, [M+H]+ found 433.9.

Step 8: The crude product was purified by SFC (column: DAICEL CHIRALCEL OD(250 mm*50 mm, 10 um); mobile phase: [CO2-ACN/i-PrOH(0.1% NH3H2O)]; B %:62.5%, isocratic elution mode) to give (6S*)—N-[5-[5-[(1R,2S)-2-fluorocyclopropyl]-1,2,4-oxadiazol-3-yl]-2-methyl-phenyl]-6-methyl-5-oxa-1,10-diazatricyclo[7.3.0.03,7]dodeca-2,7,9,11-tetraene-12-carboxamide (I-470) (154.76 mg, 31% yield, 100% purity) as off-white solid. ¹H NMR (400 MHz, DMSO-d₆) δ 9.99 (s, 1H), 9.42 (s, 1H), 8.55 (s, 1H), 8.03 (s, 1H), 7.77 (br d, J=8.0 Hz, 1H), 7.68 (s, 1H), 7.48 (br d, J=8.0 Hz, 1H), 5.40-5.17 (m, 2H), 5.14-4.92 (m, 2H), 3.12-2.99 (m, 1H), 2.35 (s, 3H), 2.02-1.87 (m, 1H), 1.58 (m, 1H), 1.50 (br d, J=6.4 Hz, 3H). MS (ESI): m/z for C₂₂H₁₇N₆O₂F [M+H]+ calcd. 434.2, [M+H]+ found 434.2.

Step 8a: The crude product was purified by SFC (column: DAICEL CHIRALCEL OD(250 mm*50 mm, 10 um); mobile phase: [CO2-ACN/i-PrOH(0.1% NH3H2O)]; B %:62.5%, isocratic elution mode) to give (6R*)—N-[5-[5-[(1R,2S)-2-fluorocyclopropyl]-1,2,4-oxadiazol-3-yl]-2-methyl-phenyl]-6-methyl-5-oxa-1,10-diazatricyclo[7.3.0.03,7]dodeca-2,7,9,11-tetraene-12-carboxamide (I-471) (142.52 mg, 28.50% yield, 100% purity) as off-white solid. ¹H NMR (400 MHz, DMSO-d₆) δ 9.99 (s, 1H), 9.43 (s, 1H), 8.55 (s, 1H), 8.04 (s, 1H), 7.77 (d, J=8.0 Hz, 1H), 7.69 (s, 1H), 7.48 (d, J=8.0 Hz, 1H), 5.40-5.17 (m, 2H), 5.14-4.92 (m, 2H), 3.11-2.99 (m, 1H), 2.35 (s, 3H), 2.02-1.86 (m, 1H), 1.63-1.53 (m, 1H), 1.50 (d, J=6.4 Hz, 3H). MS (ESI): m/z for C₂₂H₁₇N₆O₂F [M+H]+ calcd. 434.2, [M+H]+ found 434.2.

### Example 12—Preparation of N-[5-[5-[(1R,2S)-2-fluorocyclopropyl]-1,2,4-oxadiazol-3-yl]-2-methyl-phenyl]-7-(4-hydroxy-3,3-dimethyl-butyl)imidazo[1,2-a]pyridine-3-carboxamide (I-474)

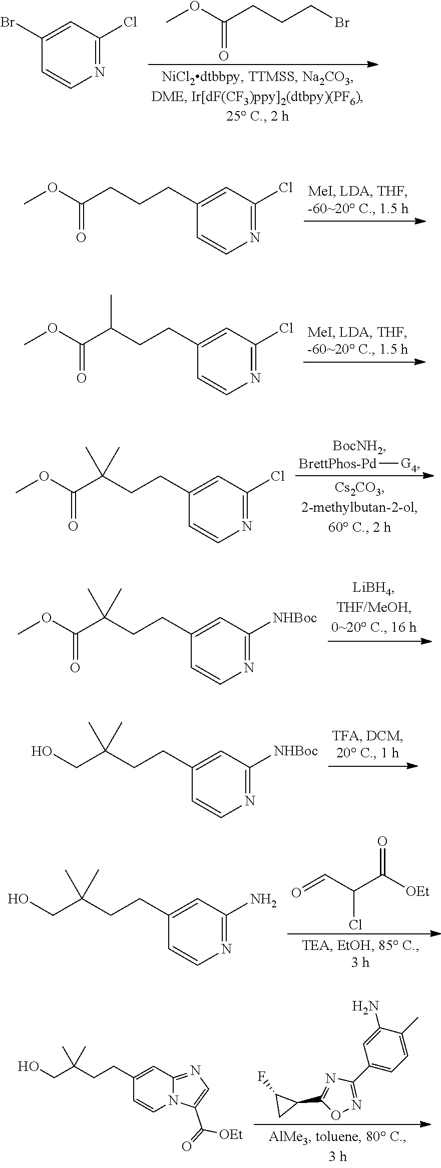

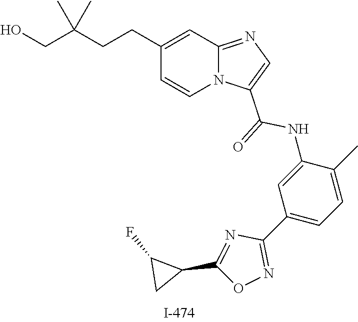 [SMILES: Cc1ccc(-c2noc([C@H]3C[C@@H]3F)n2)cc1NC(=O)c1cnc2cc(CCC(C)(C)CO)ccn12]

Step 1: To a mixture of 4-bromo-2-chloro-pyridine (5 g, 25.9 mmol, 1 eq), methyl 4-bromobutanoate (6.11 g, 33.7 mmol, 1.3 eq), Ir[dF(CF₃)ppy]₂(dtbpy)(PF₆) (291 mg, 259 μmol, 0.01 eq), TTMSS (6.46 g, 25.9 mmol, 8.02 mL, 1 eq) and Na₂CO₃ (5.51 g, 51.9 mmol, 2 eq) in DME (100 mL) was added NiCl₂.dtbbpy (51.7 mg, 129 μmol, 0.005 eq). The reaction mixture was stirred at 25° C. for 2 hrs under nitrogen atmosphere and irradiated with a 455 nm blue LED. The reaction mixture was filtered. The filtrate was diluted with water (200 mL) and extracted with ethyl acetate (100 mL×3). The combined organic layers were washed with brine (200 mL×3). The organic layer was dried over anhydrous sodium sulfate and filtered. The filtrate was concentrated in vacuo. The residue was purified by reversed-phase HPLC (0.1% FA condition) and column chromatography (SiO₂, petroleum ether:ethyl acetate=5:1) to give methyl 4-(2-chloro-4-pyridyl)butanoate (2.6 g, 12.1 mmol, 46.8% yield) as a colorless oil. ¹H NMR (400 MHz, DMSO-d₆) δ: 8.29 (d, J=5.2 Hz, 1H), 7.38 (s, 1H), 7.27 (d, J=5.2 Hz, 1H), 3.58 (s, 3H), 2.63 (t, J=7.6 Hz, 2H), 2.35-2.29 (m, 2H), 1.91-1.78 (m, 2H).

Step 2: To a mixture of methyl 4-(2-chloro-4-pyridyl)butanoate (1 g, 4.68 mmol, 1 eq) in THE (10 mL) was added LDA (2 M, 3.51 mL, 1.5 eq) at −70° C. The mixture was stirred at −60° C. for 0.5 hr. Then Mel (996 mg, 7.02 mmol, 437 μL, 1.5 eq) was added at −60° C. The reaction mixture was stirred at 20° C. for 1 hr under nitrogen atmosphere. The reaction mixture was quenched by addition saturated NH₄Cl (10 mL) at 0° C. The reaction mixture was diluted with water (50 mL) and extracted with ethyl acetate (30 mL×3). The combined organic layers were washed with brine (50 mL×3). The organic layer was dried over anhydrous sodium sulfate and filtered. The filtrate was concentrated in vacuo. The residue was purified by reversed-phase HPLC (0.1% NH₃·H₂O condition) to give methyl 4-(2-chloro-4-pyridyl)-2-methyl-butanoate (300 mg, 1.32 mmol, 28.1% yield) as a red oil. ¹H NMR (400 MHz, DMSO-d₆) δ: 8.29 (d, J=5.2 Hz, 1H), 7.38 (s, 1H), 7.30-7.24 (m, 1H), 3.59 (s, 3H), 2.65-2.58 (m, 2H), 2.47-2.39 (m, 1H), 1.92-1.79 (m, 1H), 1.75-1.65 (m, 1H), 1.11 (d, J=7.2 Hz, 3H).

Step 3: To a mixture of methyl 4-(2-chloro-4-pyridyl)-2-methyl-butanoate (300 mg, 1.32 mmol, 1 eq) in THE (5 mL) was added LDA (2 M, 790 μL, 1.2 eq) at −60° C. The mixture was stirred at −60° C. for 0.5 hr. Then Mel (187 mg, 1.32 mmol, 82.0 μL, 1 eq) was added at −60° C. The reaction mixture was stirred at 20° C. for 1 hr under nitrogen atmosphere. The reaction mixture was quenched by addition saturated NH₄Cl (10 mL) at 0° C. The reaction mixture was diluted with water (50 mL) and extracted with ethyl acetate (30 mL×3). The combined organic layers were washed with brine (50 mL×3). The organic layer was dried over anhydrous sodium sulfate and filtered. The filtrate was concentrated in vacuo. The residue was purified by reversed-phase HPLC (0.1% NH₃·H₂O condition) to give methyl 4-(2-chloro-4-pyridyl)-2,2-dimethyl-butanoate (190 mg, 786 μmol, 59.6% yield) as a red oil. ¹H NMR (400 MHz, DMSO-d₆) δ: 8.28 (d, J=5.2 Hz, 1H), 7.37 (s, 1H), 7.26 (dd, J=1.2, 5.2 Hz, 1H), 3.59 (s, 3H), 2.57-2.51 (m, 2H), 1.82-1.73 (m, 2H), 1.18 (s, 6H).

Step 4: To a mixture of methyl 4-(2-chloro-4-pyridyl)-2,2-dimethyl-butanoate (170 mg, 703 μmol, 1 eq), NH₂Boc (247 mg, 2.11 mmol, 3 eq) and Cs₂CO₃ (687 mg, 2.11 mmol, 3 eq) in 2-methylbutan-2-ol (5 mL) was added BrettPhos (Pd, G₄) (64.7 mg, 70.3 μmol, 0.1 eq). The reaction mixture was stirred at 60° C. for 2 hrs under nitrogen atmosphere. The reaction mixture was diluted with water (50 mL) and extracted with ethyl acetate (30 mL×3). The combined organic layers were washed with brine (50 mL×3). The organic layer was dried over anhydrous sodium sulfate and filtered. The filtrate was concentrated in vacuo. The residue was purified by column chromatography (SiO₂, Petroleum ether:ethyl acetate=20:1) to give methyl 4-[2-(tert-butoxycarbonylamino)-4-pyridyl]-2,2-dimethyl-butanoate (220 mg, 682 μmol, 97.0% yield) as a yellow solid. ¹H NMR (400 MHz, DMSO-d₆) δ: 9.64 (s, 1H), 8.10 (d, J=5.2 Hz, 1H), 7.64 (s, 1H), 6.85 (dd, J=1.2, 5.2 Hz, 1H), 3.61 (s, 3H), 2.46 (dd, J=4.0, 8.0 Hz, 2H), 1.78-1.72 (m, 2H), 1.47 (s, 9H), 1.18 (s, 6H).

Step 5: To a mixture of methyl 4-[2-(tert-butoxycarbonylamino)-4-pyridyl]-2,2-dimethyl-butanoate (190 mg, 589 μmol, 1 eq) in a mixed solution of THF (5 mL) and MeOH (0.5 mL) was added LiBH₄ (2 M, 2.95 mL, 10 eq) at 0° C. The reaction mixture was stirred at 20° C. for 16 hrs under nitrogen atmosphere. The reaction mixture was quenched by addition saturated NH₄Cl (10 mL) at 0° C. The reaction mixture was diluted with water (50 mL) and extracted with ethyl acetate (20 mL×3). The combined organic layers were washed with brine (50 mL×3). The organic layer was dried over anhydrous sodium sulfate and filtered. The filtrate was concentrated in vacuo to give tert-butyl N-[4-(4-hydroxy-3,3-dimethyl-butyl)-2-pyridyl]carbamate (170 mg, 577 μmol, 97.9% yield) as a yellow oil. MS (ESI): m/z for C₁₆H₂₆N₂O₃ [M+H]+ calcd. 295.2, [M+H]+ found 295.2.

Step 6: To a mixture of tert-butyl N-[4-(4-hydroxy-3,3-dimethyl-butyl)-2-pyridyl]carbamate (150 mg, 509 μmol, 1 eq) in DCM (3 mL) was added TFA (1.54 g, 13.4 mmol, 1 mL). The reaction mixture was stirred at 20° C. for 1 hr. The reaction mixture was concentrated in vacuo to give 4-(2-amino-4-pyridyl)-2,2-dimethyl-butan-1-ol (150 mg, 486 mol, 95.4% yield, TFA) as a yellow oil. MS (ESI): m/z for C₁₁H₁₈N₂O [M+H]+ calcd. 195.1, [M+H]+ found 195.2.

Step 7: ethyl 7-(4-hydroxy-3,3-dimethyl-butyl)imidazo[1,2-a]pyridine-3-carboxylate. To a mixture of 4-(2-amino-4-pyridyl)-2,2-dimethyl-butan-1-ol (150 mg, 486 μmol, 1 eq, TFA) and ethyl 2-chloro-3-oxo-propanoate (109 mg, 729 μmol, 1.5 eq) in EtOH (5 mL) was added triethylamine (98.4 mg, 973 μmol, 135 μL, 2 eq). The reaction mixture was stirred at 85° C. for 3 hrs under nitrogen atmosphere. The reaction mixture was concentrated in vacuo. The residue was purified by reversed-phase HPLC (0.1% FA condition) to give ethyl 7-(4-hydroxy-3,3-dimethyl-butyl)imidazo[1,2-a]pyridine-3-carboxylate (100 mg, 344 μmol, 70.7% yield) as a yellow oil. ¹H NMR (400 MHz, DMSO-d₆) δ: 9.11 (d, J=7.2 Hz, 1H), 8.22 (s, 1H), 7.59 (s, 1H), 7.19-7.08 (m, 1H), 4.55 (t, J=5.6 Hz, 1H), 4.35 (q, J=7.2 Hz, 2H), 3.17 (d, J=5.6 Hz, 2H), 2.71-2.60 (m, 2H), 1.57-1.48 (m, 2H), 1.34 (t, J=7.2 Hz, 3H), 0.88 (s, 6H).

Step 8: To a mixture of ethyl 7-(4-hydroxy-3,3-dimethyl-butyl)imidazo[1,2-a]pyridine-3-carboxylate (80 mg, 275 μmol, 1 eq) and 5-[5-[(1R,2S)-2-fluorocyclopropyl]-1,2,4-oxadiazol-3-yl]-2-methyl-aniline (64.2 mg, 275 μmol, 1 eq) in toluene (4 mL) was added AlMe₃ (2 M, 344 μL, 2.5 eq). The reaction mixture was stirred at 80° C. for 3 hrs. The reaction mixture was quenched by saturated NH₄Cl (10 mL) at 0° C. The mixture was diluted with water (100 mL) and extracted with ethyl acetate (50 mL×3). The combined organic layers were washed with brine (100 mL×3). The organic layer was dried over anhydrous sodium sulfate and filtered. The filtrate was concentrated in vacuo. The residue was purified by Prep-HPLC (column: Phenomenex luna C18 150*25 mm*10 um; mobile phase: [water (FA)-ACN]; gradient: 25%-55% B over 10 min) to give N-[5-[5-[(1R,2S)-2-fluorocyclopropyl]-1,2,4-oxadiazol-3-yl]-2-methyl-phenyl]-7-(4-hydroxy-3,3-dimethyl-butyl)imidazo[1,2-a]pyridine-3-carboxamide (65.6 mg, 125 μmol, 45.5% yield, 100% purity, FA) as an off-white solid. ¹H NMR (400 MHz, DMSO-d₆) δ: 9.95 (s, 1H), 9.33 (d, J=7.2 Hz, 1H), 8.52 (s, 1H), 8.02 (s, 1H), 7.77 (d, J=8.0 Hz, 1H), 7.55 (s, 1H), 7.47 (d, J=8.0 Hz, 1H), 7.06 (d, J=7.2 Hz, 1H), 5.42-5.13 (m, 1H), 4.72-4.40 (m, 1H), 3.18 (s, 2H), 3.11-2.99 (m, 1H), 2.71-2.58 (m, 2H), 2.35 (s, 3H), 2.03-1.86 (m, 1H), 1.66-1.47 (m, 3H), 0.89 (s, 6H). MS (ESI): m/z for C₂₆H₂₈FN₅O₃[M+H]+ calcd. 478.2, [M+H]+ found 478.3.

### Example 13—Preparation of N-[5-[5-[(1R,2S)-2-fluorocyclopropyl]-1,2,4-oxadiazol-3-yl]-2-methyl-phenyl]-7-[2-(2-hydroxyethoxy)ethyl]imidazo[1,2-a]pyridine-3-carboxamide (I-475)

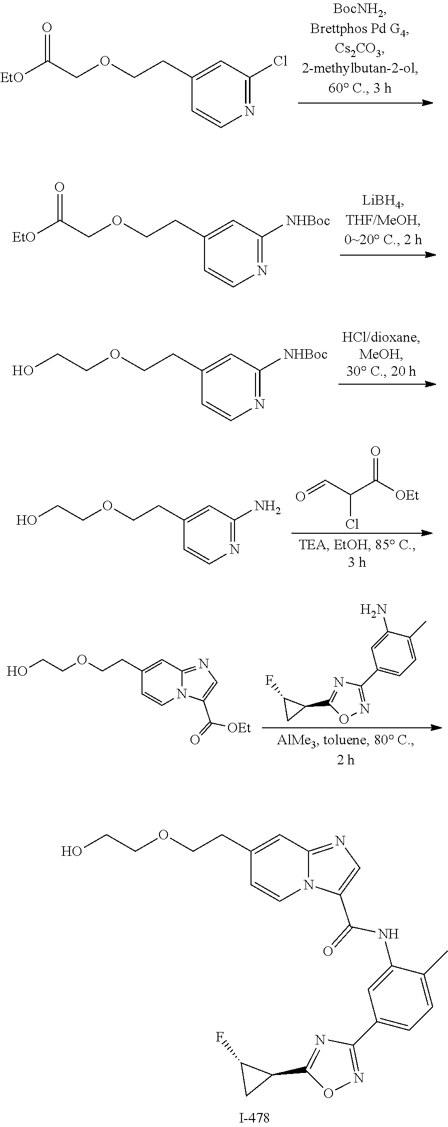

Step 1: To a mixture of ethyl 2-[2-(2-chloro-4-pyridyl)ethoxy]acetate (1 g, 4.10 mmol, 1 eq), NH₂Boc (1.44 g, 12.3 mmol, 3 eq) and Cs₂CO₃ (4.01 g, 12.3 mmol, 3 eq) in 2-methylbutan-2-ol (10 mL) was added BrettPhos (Pd, G₄) (377 mg, 410 μmol, 0.1 eq). The reaction mixture was stirred at 60° C. for 3 hrs under nitrogen atmosphere. The reaction mixture was diluted with water (100 mL) and extracted with ethyl acetate (50 mL×3). The combined organic layers were washed with brine (100 mL×3). The organic layer was dried over anhydrous sodium sulfate and filtered. The filtrate was concentrated in vacuo. The residue was purified by reversed-phase HPLC (0.1% NH₃.H₂O condition) to give ethyl 2-[2-[2-(tert-butoxycarbonylamino)-4-pyridyl]ethoxy]acetate (620 mg, 1.91 mmol, 46.5% yield) as a yellow oil. ¹H NMR (400 MHz, DMSO-d₆) δ: 9.64 (s, 1H), 8.11 (d, J=5.2 Hz, 1H), 7.68 (s, 1H), 6.94 (dd, J=1.2, 5.2 Hz, 1H), 4.14-4.06 (m, 4H), 3.71 (t, J=6.4 Hz, 2H), 2.83 (t, J=6.4 Hz, 2H), 1.47 (s, 9H), 1.21-1.16 (m, 3H).

Step 2: To a mixture of ethyl 2-[2-[2-(tert-butoxycarbonylamino)-4-pyridyl]ethoxy]acetate (520 mg, 1.60 mmol, 1 eq) in a mixed solution of THF (10 mL) and MeOH (1 mL) was added LiBH₄ (2 M, 8.02 mL, 10 eq) at 0° C. The reaction mixture was stirred at 20° C. for 2 hrs under nitrogen atmosphere. The reaction mixture was quenched by addition saturated NH₄Cl (10 mL) at 0° C. The reaction mixture was diluted with water (100 mL) and extracted with ethyl acetate (50 mL×3). The combined organic layers were washed with brine (100 mL×3). The organic layer was dried over anhydrous sodium sulfate and filtered. The filtrate was concentrated in vacuo to give tert-butyl N-[4-[2-(2-hydroxyethoxy)ethyl]-2-pyridyl]carbamate (450 mg, 1.59 mmol, 99.4% yield) as a yellow oil. ¹H NMR (400 MHz, DMSO-d₆) δ: 9.61 (s, 1H), 8.11 (d, J=5.2 Hz, 1H), 7.68 (s, 1H), 6.92 (d, J=5.2 Hz, 1H), 4.62 (s, 1H), 3.63 (t, J=6.8 Hz, 2H), 3.45 (s, 2H), 3.40 (s, 2H), 2.80 (t, J=6.8 Hz, 2H), 1.47 (s, 9H).

Step 3: To a mixture of tert-butyl N-[4-[2-(2-hydroxyethoxy)ethyl]-2-pyridyl]carbamate (450 mg, 1.59 mmol, 1 eq) in MeOH (5 mL) was added HCl/dioxane (4 M, 5 mL, 12.55 eq). The reaction mixture was stirred at 30° C. for 20 hrs. The reaction mixture was concentrated in vacuo to give 2-[2-(2-amino-4-pyridyl)ethoxy]ethanol (340 mg, 1.55 mmol, 97.5% yield, HCl) as a yellow oil. MS (ESI): m/z for C₉H₁₄N₂O₂ [M+H]+ calcd. 183.1, [M+H]+ found 183.2.

Step 4: To a mixture of 2-[2-(2-amino-4-pyridyl)ethoxy]ethanol (340 mg, 1.55 mmol, 1 eq, HCl) and ethyl 2-chloro-3-oxo-propanoate (351 mg, 2.33 mmol, 1.5 eq) in EtOH (5 mL) was added triethylamine (314 mg, 3.11 mmol, 432 μL, 2 eq). The reaction mixture was stirred at 85° C. for 3 hrs under nitrogen atmosphere. The reaction mixture was concentrated in vacuo. The residue was purified by reversed-phase HPLC (0.1% NH₃·H₂O condition) to give ethyl 7-[2-(2-hydroxyethoxy)ethyl]imidazo[1,2-a]pyridine-3-carboxylate (310 mg, 1.11 mmol, 71.6% yield) as a yellow solid. ¹H NMR (400 MHz, DMSO-d₆) δ: 9.11 (d, J=7.2 Hz, 1H), 8.24 (s, 1H), 7.66 (s, 1H), 7.20 (d, J=7.2 Hz, 1H), 4.58 (t, J=5.2 Hz, 1H), 4.35 (q, J=7.2 Hz, 2H), 3.71 (t, J=6.4 Hz, 2H), 3.53-3.40 (m, 4H), 2.95 (t, J=6.4 Hz, 2H), 1.34 (t, J=7.2 Hz, 3H).

Step 5: To a mixture of ethyl 7-[2-(2-hydroxyethoxy)ethyl]imidazo[1,2-a]pyridine-3-carboxylate (290 mg, 1.04 mmol, 1 eq) and 5-[5-[(1R,2S)-2-fluorocyclopropyl]-1,2,4-oxadiazol-3-yl]-2-methyl-aniline (218 mg, 937 μmol, 0.9 eq) in toluene (5 mL) was added AlMe₃ (2 M, 1.30 mL, 2.5 eq). The reaction mixture was stirred at 80° C. for 2 hrs. The reaction mixture was quenched by saturated NH₄Cl (10 mL) at 0° C. The mixture was diluted with water (100 mL) and extracted with ethyl acetate (50 mL×3). The combined organic layers were washed with brine (100 mL×3). The organic layer was dried over anhydrous sodium sulfate and filtered. The filtrate was concentrated in vacuo. The residue was purified by Prep-HPLC (column: Phenomenex luna C18 150*25 mm*10 um; mobile phase: [water (FA)-ACN]; gradient: 15%-45% B over 10 min) to give N-[5-[5-[(1R,2S)-2-fluorocyclopropyl]-1,2,4-oxadiazol-3-yl]-2-methyl-phenyl]-7-[2-(2-hydroxyethoxy)ethyl]imidazo[1,2-a]pyridine-3-carboxamide (262 mg, 546 μmol, 52.4% yield, 96.8% purity) as a white solid. ¹H NMR (400 MHz, DMSO-d₆) δ: 9.97 (s, 1H), 9.33 (d, J=7.2 Hz, 1H), 8.53 (s, 1H), 8.02 (d, J=1.6 Hz, 1H), 7.77 (dd, J=1.6, 8.0 Hz, 1H), 7.63 (s, 1H), 7.47 (d, J=8.0 Hz, 1H), 7.12 (dd, J=1.6, 7.2 Hz, 1H), 5.43-5.15 (m, 1H), 4.74-4.43 (m, 1H), 3.72 (t, J=6.4 Hz, 2H), 3.48 (d, J=4.4 Hz, 2H), 3.46-3.42 (m, 2H), 3.13-3.00 (m, 1H), 2.94 (t, J=6.4 Hz, 2H), 2.35 (s, 3H), 2.02-1.86 (m, 1H), 1.58 (qd, J=6.8, 13.2 Hz, 1H). MS (ESI): m/z for C₂₄H₂₄FN₅O₄[M+H]+ calcd. 466.2, [M+H]+ found 466.2.

### Example 14—Preparation of 2-((3-((5-(5-((1R,2S)-2-fluorocyclopropyl)-1,2,4-oxadiazol-3-yl)-2-methylphenyl)carbamoyl)imidazo[1,2-a]pyridin-7-yl)methoxy)acetic acid (I-477)

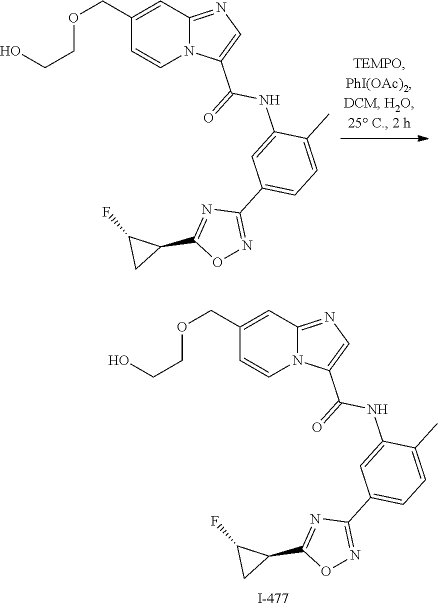 [SMILES: Cc1ccc(-c2noc([C@H]3C[C@@H]3F)n2)cc1NC(=O)c1cnc2cc(COCCO)ccn12 ; Cc1ccc(-c2noc([C@H]3C[C@@H]3F)n2)cc1NC(=O)c1cnc2cc(COCCO)ccn12]

To a solution of N-[5-[5-[(1R,2S)-2-fluorocyclopropyl]-1,2,4-oxadiazol-3-yl]-2-methyl-phenyl]-7-(2-hydroxyethoxymethyl)imidazo[1,2-a]pyridine-3-carboxamide (5.0 g, 11.1 mmol) in DCM (50 mL) and H₂O (25 mL) was added TEMPO (348 mg, 2.22 mmol) and PhI(OAc)₂ (10.7 g, 33.2 mmol). The mixture was stirred at 25° C. for 2 hours. After completion by LCMS, the mixture was quenched with H₂O (75 mL) and then extracted with DCM (3×100 mL). The combined organic phase was dried over Na₂SO₄, filtered and the filtrate was concentrated in vacuo to give a residue. The residue was purified by reverse phase IPLC (0.1% FA condition) (I-477) (3.3 g, 65.2% yield, 96% purity) was obtained as a yellow solid. LCMS: product: RT=0.582 min, m/z=466.2 (M+H)+. ¹H NMR (400 MHz, DMSO-d₆) δ: 10.01 (s, 1H), 9.40 (d, J=7.2 Hz, 1H), 8.56 (s, 1H), 8.02 (s, 1H), 7.83-7.74 (m, 1H), 7.70 (s, 1H), 7.48 (d, J=8.0 Hz, 1H), 7.15 (d, J=7.2 Hz, 1H), 5.43-5.15 (m, 1H), 4.74-4.63 (m, 2H), 4.16 (s, 2H), 3.09-3.00 (m, 1H), 2.35 (s, 3H), 2.01-1.87 (m, 1H), 1.58 (qd, J=6.4, 13.2 Hz, 1H).

### Example 15—Preparation of N-[5-[5-[(1R,2S)-2-fluorocyclopropyl]-1,2,4-oxadiazol-3-yl]-2-methyl-phenyl]-7-(3-hydroxycyclobutyl)imidazo[1,2-a]pyridine-3-carboxamide (I-478)

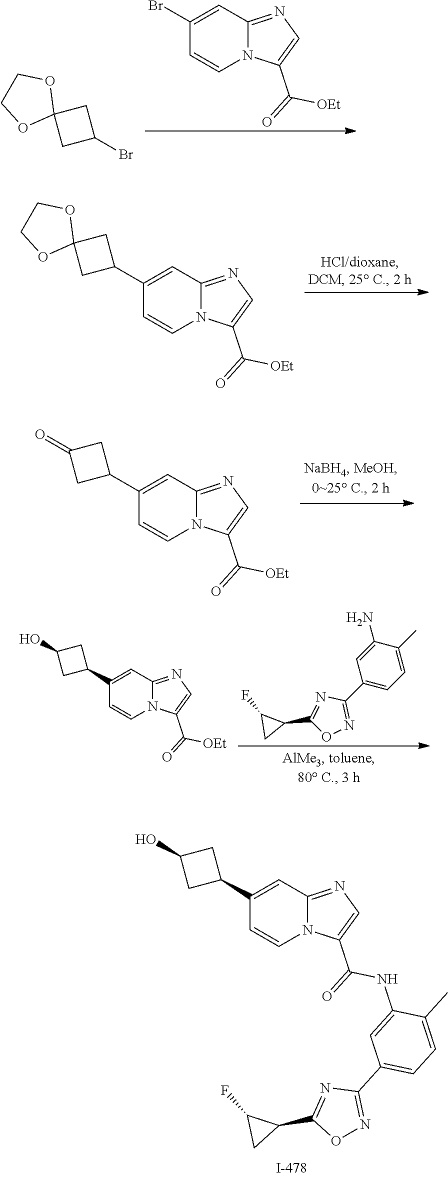

Step 1: A mixture of ethyl 7-bromoimidazo[1,2-a]pyridine-3-carboxylate (2 g, 7.43 mmol, 1 eq), 2-bromo-5,8-dioxaspiro[3.4]octane (1.87 g, 9.66 mmol, 1.3 eq), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (83.38 mg, 74.3 μmol, 0.01 eq), NiCl₂.dtbbpy (14.8 mg, 37.2 μmol, 0.005 eq) and Na₂CO₃ (1.58 g, 14.8 mmol, 2 eq) in DME (2 mL) was degassed and purged with N₂ for 3 times, and then the mixture was stirred at 25° C. for 16 hr under N₂ atmosphere at blue LED 455 nm. The mixture was concentrated to give a residue, the residue was diluted with H₂O (30 mL) and extracted with ethyl acetate (3×30 mL). The organic layer was washed with brine (30 mL), dried over with Na₂SO₄, filtered and concentrated to give a residue. The residue was purified by prep-HPLC (column: Waters Xbridge 150*25 mm*5 um; mobile phase: [water(NH₄HCO₃)-ACN]; gradient:30%-60% B over 9 min) to give ethyl 7-(5,8-dioxaspiro[3.4]octan-2-yl)imidazo[1,2-a]pyridine-3-carboxylate (1.2 g, 2.78 mmol, 37.3% yield, 70% purity) as a white solid. ¹H NMR (400 MHz, DMSO-d₆) δ: 9.15 (d, J=7.2 Hz, 1H), 8.25 (s, 1H), 7.66 (s, 1H), 7.26-7.17 (m, 1H), 4.41-4.30 (m, 2H), 3.95-3.88 (m, 2H), 3.87-3.81 (m, 2H), 3.46-3.38 (m, 1H), 2.77-2.68 (m, 2H), 2.49-2.42 (m, 2H), 1.35 (t, J=7.2 Hz, 3H).

Step 2: A mixture of ethyl 7-(5,8-dioxaspiro[3.4]octan-2-yl)imidazo[1,2-a]pyridine-3-carboxylate (300 mg, 992 μmol, 1 eq), HCl (6 M, 165 μL, 1 eq) in THF (1 mL) was degassed and purged with N₂ for 3 times, and then the mixture was stirred at 40° C. for 2 hr under N₂ atmosphere. The mixture was quenched with sat.NaHCO₃ (5 mL), diluted with H₂O (15 mL) and extracted with ethyl acetate (10 mL×3). The combined organic layers were washed with brine (30 mL), dried over Na₂SO₄, filtered and concentrated under reduced pressure to give a residue. The residue was purified by column chromatography (SiO₂, petroleum ether:ethyl acetate=3:1) to give ethyl 7-(3-oxocyclobutyl)imidazo[1,2-a]pyridine-3-carboxylate (250 mg, 968 μmol, 97.6% yield) as a white solid. ¹H NMR (400 MHz, DMSO-d₆) δ=9.23 (d, J=7.2 Hz, 1H), 8.33 (s, 1H), 7.88 (d, J=0.8 Hz, 1H), 7.39-7.34 (m, 1H), 4.46-4.38 (m, 2H), 3.92-3.84 (m, 1H), 3.58-3.53 (m, 1H), 3.53-3.50 (m, 1H), 3.43 (d, J=2.8 Hz, 2H), 1.43-1.37 (m, 3H).

Step 3: To a mixture of ethyl 7-(3-oxocyclobutyl)imidazo[1,2-a]pyridine-3-carboxylate (50 mg, 194 μmol, 1 eq) in MeOH (1 mL) was added NaBH4 (30 mg, 793 μmol, 4.10 eq). The mixture was stirred at 0° C. for 0.5 hr. The mixture was quenched with sat.aq. NH₄Cl (2 mL) and concentrated to give a residue. The residue was washed with H₂O (15 mL×3) and extracted with ethyl acetate (30 mL). The organic layer was washed with brine (15 mL), dried over Na₂SO₄, filtered and concentrated to give ethyl 7-(3-hydroxycyclobutyl)imidazo[1,2-a]pyridine-3-carboxylate (35 mg, 134 μmol, 69.5% yield) as a white solid. ¹H NMR (400 MHz, DMSO-d₆) δ=9.19 (d, J=7.2 Hz, 1H), 8.30 (s, 1H), 7.63 (s, 1H), 7.28-7.18 (m, 1H), 5.24 (d, J=7.2 Hz, 1H), 4.46-4.37 (m, 2H), 4.19-4.09 (m, 1H), 3.14-3.04 (m, 1H), 2.76-2.66 (m, 2H), 2.07-1.97 (m, 2H), 1.40 (t, J=7.2 Hz, 3H).

Step 4: To a mixture of ethyl 7-(3-hydroxycyclobutyl)imidazo[1,2-a]pyridine-3-carboxylate (150 mg, 576 μmol, 1 eq) and 5-[5-[(1R,2S)-2-fluorocyclopropyl]-1,2,4-oxadiazol-3-yl]-2-methyl-aniline (161 mg, 692 μmol, 1.2 eq) in ToI. (5 mL) was added AlMe₃ (2 M, 720 μL, 2.5 eq). The mixture was stirred at 80° C. for 3 hr. The reaction mixture was quenched by 1M HCl (5 mL) at 0° C. The mixture was diluted with water (50 mL) and basified with saturated NaHCO₃ aqueous till pH=7. The mixture was extracted with ethyl acetate (20 mL×3). The combined organic layers were washed with brine (50 mL×3). The organic layer was dried over anhydrous sodium sulfate and filtered. The filtrate was concentrated in vacuo. The residue was purified by prep-HPLC (column: Waters Xbridge 150*25 mm*5 um; mobile phase: [water(NH4HCO3)-ACN]; gradient:35%-65% B over 10 min) to give N-[5-[5-[(1R,2S)-2-fluorocyclopropyl]-1,2,4-oxadiazol-3-yl]-2-methyl-phenyl]-7-(3-hydroxycyclobutyl)imidazo[1,2-a]pyridine-3-carboxamide (I-478) (128 mg, 285 μmol, 49.55% yield, 100% purity) was obtained as a white solid. MS (ESI): m/z for C₂₄H₂₂FN₅O₃[M+H]+ calcd. 448.2, [M+H]+ found 448.3. ¹H NMR (400 MHz, DMSO-d₆) δ=9.97 (s, 1H), 9.36 (d, J=7.2 Hz, 1H), 8.54 (s, 1H), 8.03 (s, 1H), 7.78 (d, J=7.6 Hz, 1H), 7.54 (s, 1H), 7.48 (d, J=8.0 Hz, 1H), 7.11 (d, J=7.2 Hz, 1H), 5.39-5.20 (m, 1H), 5.18 (d, J=7.2 Hz, 1H), 4.15-4.04 (m, 1H), 3.12-2.98 (m, 2H), 2.71-2.60 (m, 2H), 2.36 (s, 3H), 2.02-1.89 (m, 3H), 1.64-1.54 (m, 1H).

### Example 16—Preparation of N-[5-[5-[(1R,2S)-2-fluorocyclopropyl]-1,2,4-oxadiazol-3-yl]-2-methyl-phenyl]-7-[(1S,2S)-2 (hydroxymethyl)cyclopropyl]imidazo[1,2-a]pyridine-3-carboxamide (I-480) and N-[5-[5-[(1R,2S)-2-fluorocyclopropyl]-1,2,4-oxadiazol-3-yl]-2-methyl-phenyl]-7-[(1R,2R)-2 (hydroxymethyl)cyclopropyl]imidazo[1,2-a]pyridine-3-carboxamide (I-481)

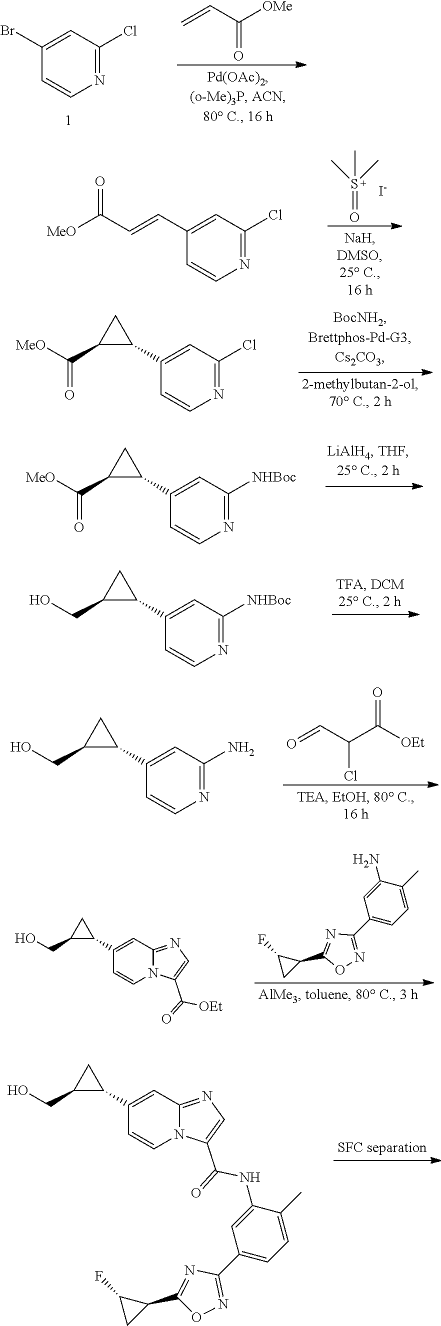

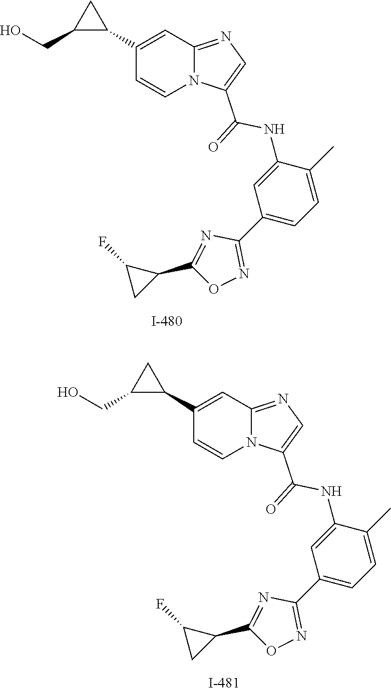 [SMILES: Cc1ccc(-c2noc([C@H]3C[C@@H]3F)n2)cc1NC(=O)c1cnc2cc([C@H]3C[C@@H]3CO)ccn12 ; Cc1ccc(-c2noc([C@H]3C[C@@H]3F)n2)cc1NC(=O)c1cnc2cc([C@@H]3C[C@H]3CO)ccn12]

Step 1: A mixture of 4-bromo-2-chloro-pyridine (20.0 g, 104 mmol, 1.0 eq), methyl prop-2-enoate (10.7 g, 125 mmol, 11.2 mL, 1.2 eq), triethylamine (12.6 g, 125 mmol, 17.4 mL, 1.2 eq) and Pd(t-Bu₃P)₂ (531 mg, 1.04 mmol, 0.01 eq) in NMP (140 mL) was degassed and purged with N₂ for 3 times, and then the mixture was stirred at 100° C. for 16 hours under N₂ atmosphere. LC-MS showed starting material consumed completely and the desired product was detected as major. The reaction mixture was quenched with water (300 mL) when a large amount of solid was precipitated from the solution. The solution was filtered, the filter cake was collected and washed with water (100 mL) and dried in vacuo to give methyl (E)-3-(2-chloro-4-pyridyl)prop-2-enoate (17.6 g, 85.7% yield) as a gray solid. ¹H NMR (400 MHz, DMSO-d₆) δ 8.45 (d, J=5.2 Hz, 1H), 7.88 (s, 1H), 7.77-7.71 (m, 1H), 7.62 (d, J=16.0 Hz, 1H), 6.98 (d, J=16.0 Hz, 1H), 3.75 (s, 3H). MS (ESI): m/z for C₉H₈ClNO₂ [M+H]⁺ calcd. 198.1, [M+H]⁺ found 198.1

Step 2: To a solution of BLAHmethane; iodide (13.9 g, 63.2 mmol, 1.00 eq) in DMSO (180 mL) was added NaH (2.53 g, 63.3 mmol, 60% purity, 1.00 eq) under N₂ atmosphere. The mixture was stirred at 25° C. for 1 hour, then methyl (E)-3-(2-chloro-4-pyridyl)prop-2-enoate (12.5 g, 63.3 mmol, 1.00 eq) in DMSO (90 mL) was added dropwise. The mixture was stirred at 25° C. for 2 hours. LC-MS showed starting material consumed completely and the desired product was detected as major. The reaction mixture was quenched by addition water (100 mL) at 0° C., and then diluted with water (150 mL) and extracted with ethyl acetate (3×150 mL). The combined organic layers were washed with brine (150 mL), dried over Na₂SO₄, filtered and concentrated under reduced pressure to give a residue. The residue was purified by prep-HPLC (mobile phase: [water (0.225% FA)-ACN]; B %: 40%-50%, 20 min) to give methyl (1S, 2S)-2-(2-chloro-4-pyridyl) cyclopropanecarboxylate (3.00 g, 11.2% yield) as a brown oil. MS (ESI): m/z for C₁₀H₁₀ClNO₂ [M+H]⁺ calcd. 212.0, [M+H]⁺ found 212.0.

Step 3: A mixture of methyl (1S,2S)-2-(2-chloro-4-pyridyl)cyclopropanecarboxylate (1.00 g, 4.72 mmol, 1.00 eq) tert-butyl carbamate (1.66 g, 14.2 mmol, 3.00 eq), Cs₂CO₃ (3.08 g, 9.45 mmol, 2.00 eq), Brettphos-Pd-G3 (435 mg, 472 μmol, 0.10 eq) in 2-methylbutan-2-ol (15 mL) was degassed and purged with N₂ for 3 times, and then the mixture was stirred at 70° C. for 2 hours under N₂ atmosphere. LC-MS showed starting material consumed completely and the desired product was detected as major. The reaction mixture was filtered, and the filtrate was concentrated in vacuo to give a residue. The residue was purified by column chromatography (SiO₂, petroleum ether/ethyl acetate=10/1 to 3/1) to give methyl (1S, 2S)-2-[2-(tert-butoxycarbonylamino)-4-pyridyl]cyclopropanecarboxylate (1.80 g, 67.8% yield, 52% purity) as a yellow solid. ¹H NMR (400 MHz, CDCl₃) δ 8.12 (d, J=5.2 Hz, 1H), 8.07-7.95 (m, 1H), 7.72 (s, 1H), 6.77-6.62 (m, 1H), 4.46 (br s, 5H), 3.72 (s, 3H), 2.54-2.46 (m, 1H), 2.04-1.98 (m, 1H), 1.70-1.64 (m, 1H), 1.53 (s, 9H), 1.46 (s, 21H), 1.44-1.39 (m, 1H). MS (ESI): m/z for C₁₅H₂₀N₂O₄ [M+H-56]⁺ calcd. 237.1, [M+H-56]⁺ found 237.1.

Step 4: To a solution of methyl (1S, 2S)-2-[2-(tert-butoxycarbonylamino)-4-pyridyl]cyclopropanecarboxylate (900 mg, 1.60 mmol, 1.00 eq) in THE (10 mL) was added LiAlH₄ (2.5 M, 961 μL, 1.50 eq). The mixture was stirred at 0° C. for 2 hours. After completion detected by LC-MS, the reaction mixture was cooled to 0° C., and the reaction mixture was quenched by addition H₂O (2.0 mL), followed by 15% aqueous NaOH (2.0 mL) and H₂O (6.0 mL). The mixture was stirred at room temperature for 0.5 hour and then concentrated to give a residue. The residue was washed with (DCM/MeOH=5/1, 100 mL), the filtrate was concentrated in vacuo to give a crude product. The crude product was purified by column chromatography (SiO₂, petroleum ether/ethyl acetate=5/1 to 1/1) to give tert-butyl N-[4-[(1S,2S)-2-(hydroxymethyl)cyclopropyl]-2-pyridyl]carbamate (510 mg, 60.3% yield) as a white solid. MS (ESI): m/z for C₁₄H₂₀N₂O₃ [M+H-56]+ calcd. 209.0, [M+H-56]+ found 209.0. ¹H NMR (400 MHz, DMSO-d₆) δ 9.58 (s, 1H), 8.02 (d, J=5.2 Hz, 1H), 7.54 (s, 1H), 6.74-6.60 (m, 1H), 4.65 (t, J=5.6 Hz, 1H), 3.52-3.43 (m, 1H), 3.38-3.33 (m, 1H), 1.83-1.75 (m, 1H), 1.46 (s, 9H), 1.41-1.28 (m, 1H), 1.03-0.94 (m, 1H), 0.94-0.88 (m, 1H).

Step 5: To a solution of tert-butyl N-[4-[(1 S, 2S)-2-(hydroxymethyl)cyclopropyl]-2-pyridyl]carbamate (450 mg, 1.70 mmol, 1.00 eq) in DCM (5.0 mL) was added TFA (1.5 mL). The mixture was stirred at 25° C. for 1 hour. After completion detected by LC-MS, the reaction mixture was concentrated in vacuo to give [(1S, 2S)-2-(2-amino-4-pyridyl)cyclopropyl]methanol (473 mg, crude, TFA salt) as a yellow oil. MS (ESI): m/z for C₉H₁₂N₂O [M+H]⁺ calcd. 165.2, [M+H]⁺ found 165.2.

Step 6: To a solution of [(1S,2S)-2-(2-amino-4-pyridyl)cyclopropyl]methanol (473 mg, 1.70 mmol, 1.00 eq, TFA salt) in EtOH (6.0 mL) was added triethylamine (860 mg, 8.50 mmol, 1.18 mL, 5.0 eq) and ethyl 2-chloro-3-oxo-propanoate (307 mg, 2.04 mmol, 1.2 eq). The mixture was stirred at 80° C. for 16 hours. After completion detected by LC-MS, the reaction mixture was concentrated in vacuo to give a residue. The residue was purified by column chromatography (SiO₂, petroleum ether/ethyl acetate=5/1 to 1/1) to give ethyl 7-[(1S, 2S)-2-(hydroxymethyl)cyclopropyl]imidazo[1,2-a]pyridine-3-carboxylate (270 mg, 61.0% yield) as a yellow solid. MS (ESI): m/z for C₁₄H₁₆N₂O₃ [M+H]+ calcd. 261.1, [M+H]+ found 261.1. ¹H NMR (400 MHz, CDCl₃) δ 9.15 (d, J=7.2 Hz, 1H), 8.24 (s, 1H), 7.41 (s, 1H), 6.76 (br d, J=6.8 Hz, 1H), 4.53-4.30 (m, 2H), 3.82-3.54 (m, 2H), 2.00-1.92 (m, 1H), 1.62-1.53 (m, 1H), 1.42 (t, J=7.2 Hz, 3H), 1.11 (t, J=7.2 Hz, 2H).

Step 7: To a solution of ethyl 7-[(1S,2S)-2-(hydroxymethyl)cyclopropyl]imidazo[1,2-a]pyridine-3-carboxylate (180 mg, 692 μmol, 1.00 eq) and 5-[5-[(1R,2S)-2-fluorocyclopropyl]-1,2,4-oxadiazol-3-yl]-2-methyl-aniline (161 mg, 692 μmol, 1.0 eq) in toluene (1.0 mL) was added AlMe₃ (2 M, 864 μL, 2.5 eq). The mixture was stirred at 80° C. for 1 hour. After completion detected by LC-MS, the reaction mixture was quenched by H₂O (10 mL) at 25° C. The mixture was diluted with water (10 mL) and was extracted with ethyl acetate (3×10 mL). The combined organic layers were washed with brine (3×10 mL). The organic layer was dried over anhydrous sodium sulfate and filtered, the filtrate was concentrated in vacuo to give a residue. The residue was purified by prep-HPLC (mobile phase: [water (0.225% FA)-ACN]; B %: 40%-50%, 10 min) to afford N-[5-[5-[(1R, 2S)-2-fluorocyclopropyl]-1,2,4-oxadiazol-3-yl]-2-methyl-phenyl]-7-[(1S,2S)-2 (hydroxymethyl)cyclopropyl]imidazo[1,2-a]pyridine-3-carboxamide (280 mg, 90.5% yield) was obtained as a yellow solid. MS (ESI): m/z for C₂₄H₂₂FN₅O₃[M+H]⁺ calcd. 448.0, [M+H]⁺ found. 448.0. ¹H NMR (400 MHz, DMSO-d₆) δ 10.03 (s, 1H), 9.29 (d, J=7.2 Hz, 1H), 8.57 (s, 1H), 8.01 (s, 1H), 7.76 (d, J=8.0 Hz, 1H), 7.48 (s, 1H), 6.91 (br d, J=7.2 Hz, 1H), 5.45-5.14 (m, 1H), 4.86-4.55 (m, 1H), 3.59-3.48 (m, 1H), 3.41-3.33 (m, 2H), 3.11-3.00 (m, 1H), 2.35 (s, 3H), 2.01-1.88 (m, 2H), 1.64-1.51 (m, 1H), 1.50-1.41 (m, 1H), 1.15-0.93 (m, 2H).

Step 8: The residue was separated by SFC (column: DAICEL CHIRALPAK IE (250 mm*30 mm, 10 um); mobile phase: [Hexane-EtOH]; B %:100%, isocratic elution mode). Then the two peaks were purified by prep-HPLC (column: Waters Xbridge 150*25 mm*5 um; mobile phase: [water (ammonia hydroxide v/v)-ACN]; gradient:30%-60% B over 10 min) and prep-HPLC (column: Waters Xbridge 150*25 mm*5 um; mobile phase: [water (ammonia hydroxide v/v)-ACN]; gradient: 30%-60% B over 10 min). Compound N-[5-[5-[(1R,2S)-2-fluorocyclopropyl]-1,2,4-oxadiazol-3-yl]-2-methyl-phenyl]-7-[(1S,2S)-2 (hydroxymethyl)cyclopropyl]imidazo[1,2-a]pyridine-3-carboxamide (I-480)(64.5 mg, 23.0% yield, 100% purity) was obtained as a white solid. Optical rotation R_{T}=3.086 min specific rotation=−32.771°. MS (ESI): m/z for C₂₄H₂₂FN₅O₃[M+H]⁺ calcd. 448.0, [M+H]⁺ found 448.0. ¹H NMR (400 MHz, DMSO-d₆) δ 9.92 (s, 1H), 9.28 (d, J=7.2 Hz, 1H), 8.50 (s, 1H), 8.02 (d, J=1.6 Hz, 1H), 7.83-7.71 (m, 1H), 7.52-7.41 (m, 2H), 6.99-6.83 (m, 1H), 5.42-5.14 (m, 1H), 4.68 (t, J=5.6 Hz, 1H), 3.56-3.48 (m, 1H), 3.42-3.35 (m, 1H), 3.11-2.99 (m, 1H), 2.34 (s, 3H), 2.01-1.88 (m, 2H), 1.64-1.54 (m, 1H), 1.51-1.41 (m, 1H), 1.11-0.97 (m, 2H),

Compound N-[5-[5-[(1R,2S)-2-fluorocyclopropyl]-1,2,4-oxadiazol-3-yl]-2-methyl-phenyl]-7-[(1R,2R)-2-(hydroxymethyl)cyclopropyl]imidazo[1,2-a]pyridine-3-carboxamide (I-481) (52.9 mg, 18.9% yield, 100% purity) was obtained as a white solid. Optical rotation R_{T}=5.204 min, specific rotation=+49.778°. MS (ESI): m/z for C₂₄H₂₂FN₅O₃[M+H]⁺ calcd. 448.0, [M+H]⁺ found 448.0. ¹H NMR (400 MHz, DMSO-d₆) δ 9.92 (s, 1H), 9.29 (d, J=7.2 Hz, 1H), 8.51 (s, 1H), 8.03 (d, J=1.6 Hz, 1H), 7.83-7.69 (m, 1H), 7.54-7.40 (m, 2H), 6.99-6.85 (m, 1H), 5.46-5.13 (m, 1H), 4.69 (t, J=5.6 Hz, 1H), 3.59-3.47 (m, 1H), 3.42-3.36 (m, 1H), 3.15-3.00 (m, 1H), 2.35 (s, 3H), 2.03-1.87 (m, 2H), 1.66-1.54 (m, 1H), 1.52-1.41 (m, 1H), 1.09-0.98 (m, 2H).

### Example 17—Preparation of (4S*)—N-[5-[5-[(1R,2S)-2-fluorocyclopropyl]-1,2,4-oxadiazol-3-yl]-2-methyl-phenyl]-4-methyl-5-oxa-1,10-diazatricyclo[7.3.0.03′⁷]dodeca-2,7,9,11-tetraene-12-carboxamide (I-482) and (4R*)—N-[5-[5-[(1R,2S)-2-fluorocyclopropyl]-1,2,4-oxadiazol-3-yl]-2-methyl-phenyl]-4-methyl-5-oxa-1,10-diazatricyclo[7.3.0.03′⁷]dodeca-2,7,9,11-tetraene-12-carboxamide (I-483)

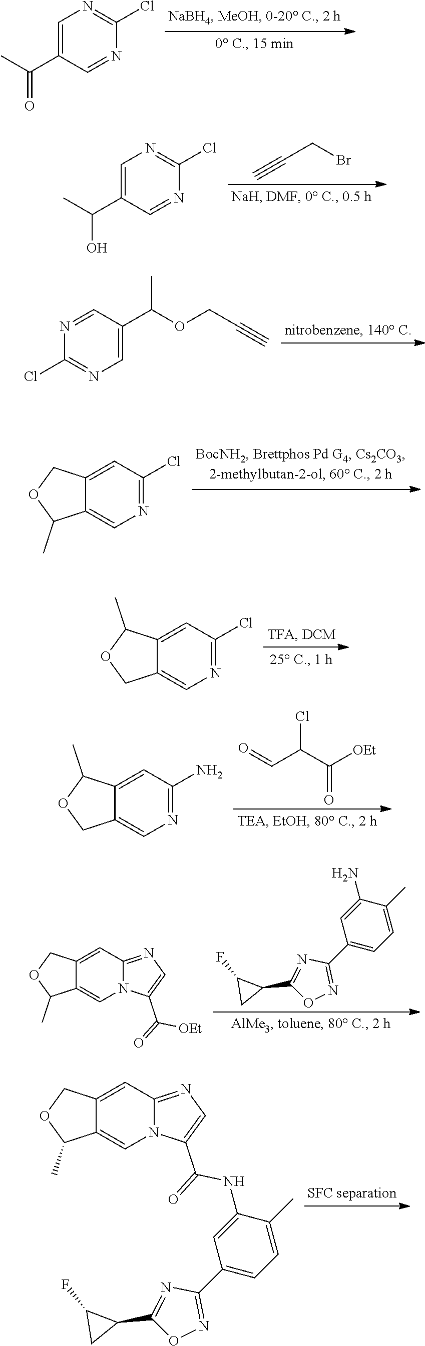

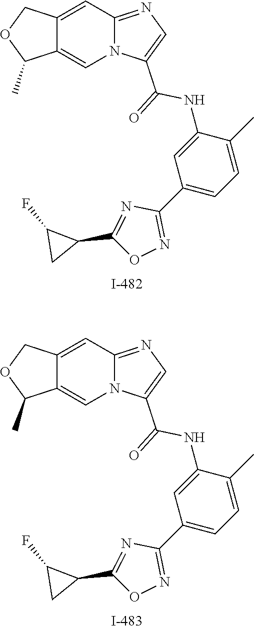 [SMILES: Cc1ccc(-c2noc([C@H]3C[C@@H]3F)n2)cc1NC(=O)c1cnc2cc3c(cn12)[C@H](C)OC3 ; Cc1ccc(-c2noc([C@H]3C[C@@H]3F)n2)cc1NC(=O)c1cnc2cc3c(cn12)[C@@H](C)OC3]

Step 1: To a mixture of 1-(2-chloropyrimidin-5-yl)ethanone (4 g, 25.5 mmol, 1 eq) in MeOH (15 mL) was added NaBH₄ (1.88 g, 49.6 mmol, 1.95 eq) at 0° C. The mixture was stirred at 0° C. for 15 min. The mixture was quenched with NH₄Cl (5 mL) and concentrated to give a residue. The residue was washed with H₂O (3×15 mL) and extracted with ethyl acetate (30 mL). The organic layer was washed with brine (15 mL), dried over Na₂SO₄, filtered and concentrated to give a residue. The residue was purified by column chromatography (SiO₂, petroleum ether:ethyl acetate=1:1) to give 1-(2-chloropyrimidin-5-yl)ethanol (0.9 g, 5.68 mmol, 22.2% yield) as a white solid. ¹H NMR (400 MHz, DMSO-d₆) δ=8.75 (s, 2H), 5.59 (d, J=4.4 Hz, 1H), 4.90-4.81 (m, 1H), 1.39 (d, J=6.4 Hz, 3H).

Step 2: To a mixture of 1-(2-chloropyrimidin-5-yl)ethanol (0.8 g, 5.04 mmol, 1 eq) and 3-bromoprop-1-yne (975 mg, 6.56 mmol, 706 μL, 1.3 eq) in DMF (20 mL) was added at 0° C. NaH (221 mg, 5.55 mmol, 60% purity, 1.1 eq) was added and stirred at 0° C. under N₂. The mixture was quenched with NH₄Cl (2 mL). The mixture was concentrated to give a residue. The mixture was purified by column chromatography (SiO₂, petroleum ether:ethyl acetate=5:1) to give 2-chloro-5-(1-prop-2-ynoxyethyl)pyrimidine (0.45 g, 2.29 mmol, 45.3% yield) was obtained as a white solid.

Step 3: A mixture of 2-chloro-5-(1-prop-2-ynoxyethyl)pyrimidine (0.45 g, 2.29 mmol, 1 eq) in nitrobenzene (10 mL) was stirred at 140° C. for 10 hr. The mixture was concentrated to give a residue. The mixture was purified by column chromatography (SiO₂, petroleum ether:ethyl acetate=1:0˜ 5:1) to give 6-chloro-3-methyl-1,3-dihydrofuro[3,4-c]pyridine (0.35 g, 2.06 mmol, 90.1% yield) as a white solid. H NMR (400 MHz, DMSO-d₆) δ=8.36 (s, 1H), 7.50 (s, 1H), 5.29 (q, J=6.4 Hz, 1H), 5.08-4.99 (m, 1H), 4.96-4.89 (m, 1H), 1.44 (d, J=6.4 Hz, 3H).

Step 4: A mixture of 6-chloro-3-methyl-1,3-dihydrofuro[3,4-c]pyridine (380 mg, 2.24 mmol, 1 eq), NH₂Boc (787 mg, 6.72 mmol, 3 eq), BrettPhos (Pd, G4) (206 mg, 224 μmol, 0.1 eq) and Cs₂CO₃ (2.19 g, 6.72 mmol, 3 eq) in 2-methylbutan-2-ol (8 mL) was stirred at 60° C. for 2 hr under N₂. The reaction mixture was diluted with H₂O (30 mL) and extracted with ethyl acetate (30 mL×3). The combined organic layers were washed with brine (30 mL) dried over Na₂SO₄, the mixture was filtered and concentrated in vacuo. The residue was purified by column chromatography (SiO₂, petroleum ether:ethyl acetate=3:1) to give tert-butyl N-(1-methyl-1,3-dihydrofuro[3,4-c]pyridin-6-yl)carbamate (400 mg, 1.04 mmol, 46.3% yield, 65% purity) as white solid. MS (ESI): m/z for C₁₃H₁₈N₂O₃ [M+H-56]⁺ calcd. 195.1, [M+H]⁺ found 195.1.

Step 5: To a mixture of tert-butyl N-(1-methyl-1,3-dihydrofuro[3,4-c]pyridin-6-yl)carbamate (300 mg, 1.20 mmol, 1 eq) in DCM (6 mL) was added TFA (4.61 g, 40.3 mmol, 3.00 mL, 33.6 eq). The mixture was stirred at 25° C. for 1 hr. The reaction mixture was concentrated in vacuo to give 1-methyl-1,3-dihydrofuro[3,4-c]pyridin-6-amine (300 mg, 1.14 mmol, 94.7% yield, TFA) as white solid. MS (ESI): m/z for C₈H₁₀N₂O [M+H]⁺ calcd. 151.0, [M+H]⁺ found 151.2.

Step 6: To a mixture of 1-methyl-1,3-dihydrofuro[3,4-c]pyridin-6-amine (300 mg, 1.14 mmol, 1 eq, TFA) in EtOH (5 mL) was added triethylamine (344 mg, 3.41 mmol, 474 μL, 3 eq), then ethyl 2-chloro-3-oxo-propanoate (205 mg, 1.36 mmol, 1.2 eq) was added and stirred at 80° C. for 2 hr. The reaction mixture was diluted with H₂O (30 mL) and extracted with ethyl acetate (30 mL×3). The combined organic layers were washed with brine (30 mL) dried over Na₂SO₄, filtered and concentrated in vacuo. The residue was purified by column chromatography (SiO₂, petroleum ether:ethyl acetate=2:1) to give ethyl 4-methyl-5-oxa-1,10-diazatricyclo[7.3.0.0^{3,7}]dodeca-2,7,9,11-tetraene-12-carboxylate (150 mg, 609 μmol, 53.6% yield) as yellow solid. ¹H NMR (400 MHz, DMSO-d₆) δ=9.15 (s, 1H), 8.27 (s, 1H), 7.71 (d, J=0.8 Hz, 1H), 5.34 (q, J=6.4 Hz, 1H), 5.14-5.06 (m, 1H), 5.00-4.93 (m, 1H), 4.37 (q, J=7.2 Hz, 2H), 1.53-1.48 (m, 3H), 1.35 (t, J=7.2 Hz, 3H).

Step 7: To a mixture of ethyl 4-methyl-5-oxa-1,10-diazatricyclo[7.3.0.0^{3,7}]dodeca-2,7,9,11-tetraene-12-carboxylate (130 mg, 527 μmol, 1 eq) and 5-[5-[(1R,2S)-2-fluorocyclopropyl]-1,2,4-oxadiazol-3-yl]-2-methyl-aniline (123 mg, 527 μmol, 1 eq) in ToI. (4 mL) was added AlMe₃ (2.5 M, 527 μL, 2.5 eq). The mixture was stirred at 80° C. for 2 hr. The reaction mixture was quenched by water (10 mL). The mixture was diluted with water (20 mL) and extracted with ethyl acetate (10 mL×3). The combined organic layers were washed with brine (30 mL×3). The organic layer was dried over anhydrous sodium sulfate and filtered. The filtrate was concentrated in vacuo. The residue was purified by column chromatography (SiO₂, petroleum ether:ethyl acetate=1:5) to give N-[5-[5-[(1R,2S)-2-fluorocyclopropyl]-1,2,4-oxadiazol-3-yl]-2-methyl-phenyl]-4-methyl-5-oxa-1,10-diazatricyclo[7.3.0.0^{3,7}]dodeca-2,7,9,11-tetraene-12-carboxamide (130 mg, 299 μmol, 56.8% yield) as white solid. ¹H NMR (400 MHz, DMSO-d₆) δ=10.01 (s, 1H), 9.37 (s, 1H), 8.56 (s, 1H), 8.04 (d, J=1.6 Hz, 1H), 7.78 (m, 1H), 7.68 (d, J=1.2 Hz, 1H), 7.49 (d, J=7.6 Hz, 1H), 5.40-5.20 (m, 2H), 5.13-5.06 (m, 1H), 5.00-4.93 (m, 1H), 3.13-3.02 (m, 1H), 2.36 (s, 3H), 2.01-1.88 (m, 1H), 1.65-1.54 (m, 1H), 1.49 (d, J=6.4 Hz, 3H).

Step 8: The crude product was purified by reversed-phase SFC (column: DAICEL CHIRALPAK AD (250 mm*30 mm, 10 um); mobile phase: [CO₂-ACN/EtOH(0.1% NH₃H₂O)]; B %:0%, isocratic elution mode). (4S*)—N-[5-[5-[(1R,2S)-2-fluorocyclopropyl]-1,2,4-oxadiazol-3-yl]-2-methyl-phenyl]-4-methyl-5-oxa-1,10-diazatricyclo[7.3.0.0^{3,7}]dodeca-2,7,9,11-tetraene-12-carboxamide (I-482) (56.6 mg, 130 μmol, 43.5% yield, 100% purity) was obtained as white solid. ¹H NMR (DMSO-d₆) δ: 9.98 (s, 1H), 9.35 (s, 1H), 8.55 (s, 1H), 8.03 (d, J=1.6 Hz, 1H), 7.76 (m, 1H), 7.66 (d, J=1.2 Hz, 1H), 7.47 (d, J=8.0 Hz, 1H), 5.23 (s, 2H), 5.03 (s, 1H), 4.91-4.99 (m, 1H), 2.99-3.11 (m, 1H), 2.35 (s, 3H), 1.87-2.01 (m, 1H), 1.52-1.63 (m, 1H), 1.47 (d, J=6.4 Hz, 3H).

(4R*)—N-[5-[5-[(1R,2S)-2-fluorocyclopropyl]-1,2,4-oxadiazol-3-yl]-2-methyl-phenyl]-4-methyl-5-oxa-1,10-diazatricyclo[7.3.0.0^{3,7}]dodeca-2,7,9,11-tetraene-12-carboxamide (I-483) (56.9 mg, 131 μmol, 43.7% yield, 100% purity) was obtained as white solid. ¹H NMR (DMSO-d₆) δ: 9.99 (s, 1H), 9.36 (s, 1H), 8.56 (s, 1H), 8.04 (d, J=1.6 Hz, 1H), 7.77 (m, 1H), 7.67 (s, 1H), 7.48 (d, J=8.0 Hz, 1H), 5.20-5.39 (m, 2H), 5.06-5.12 (m, 1H), 4.92-4.99 (m, 1H), 3.06 (s, 1H), 2.36 (s, 3H), 1.86-1.98 (m, 1H), 1.54-1.64 (m, 1H), 1.48 (d, J=6.4 Hz, 3H).

### Example 18—Preparation of 6-fluoro-N-(5-(5-((1R,2S)-2-fluorocyclopropyl)-1,2,4-oxadiazol-3-yl)-2-methylphenyl)pyrazolo[1,5-a]pyridine-3-carboxamide (I-486)

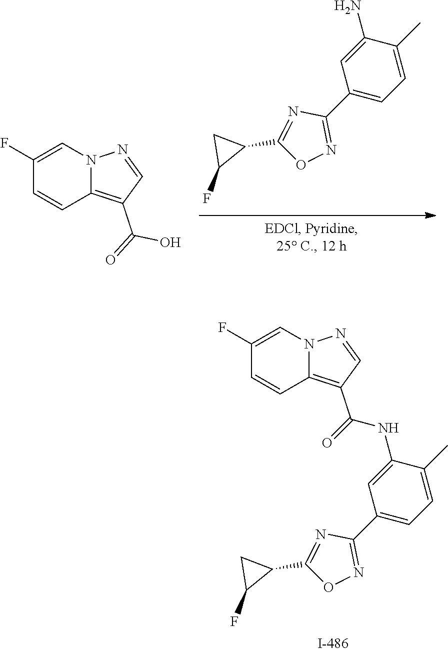 [SMILES: O=C(O)c1cnn2cc(F)ccc12 ; Cc1ccc(-c2noc([C@H]3C[C@@H]3F)n2)cc1N ; Cc1ccc(-c2noc([C@H]3C[C@@H]3F)n2)cc1NC(=O)c1cnn2cc(F)ccc12]

Step 1: To a solution of 6-fluoropyrazolo[1,5-a]pyridine-3-carboxylic acid (150 mg, 832.71 μmol, 1 eq) in pyridine (4 mL) was added EDCI (255 mg, 1.33 mmol, 1.6 eq) at 25° C., the mixture was stirred for 2 hours. Then 5-[5-[(1R,2S)-2-fluorocyclopropyl]-1,2,4-oxadiazol-3-yl]-2-methyl-aniline (194 mg, 832.71 μmol, 1.0 eq) was added. The mixture was stirred at 25° C. for 10 hours. The reaction mixture was partitioned between H₂O (30 mL) and ethyl acetate (50 mL). The organic phase was separated, washed with 0.05 N HCl 60 mL (30 mL×2), dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; gradient: 42%-72% B over 10 min) to give 6-fluoro-N-(5-(5-((1R,2S)-2-fluorocyclopropyl)-1,2,4-oxadiazol-3-yl)-2-methylphenyl)pyrazolo[1,5-a]pyridine-3-carboxamide (I-486) (50.41 mg, 15.22% yield) as a white solid. ¹H NMR (400 MHz, DMSO-d6) δ=10.12 (s, 1H), 9.56 (s, 1H), 8.64 (s, 1H), 8.02 (d, J=1.6 Hz, 1H), 7.88-7.75 (m, 2H), 7.60-7.55 (m, 1H), 7.50 (d, J=8.0 Hz, 1H), 5.40-5.18 (m, 1H), 3.11-3.04 (m, 1H), 3.03 (s, 6H), 2.36 (s, 3H), 2.04-1.85 (m, 1H), 1.70-1.44 (m, 1H). MS (ESI): m/z for C₂₅H₂₃FN₆O₃[M+H]+ calcd. 396.4 [M+H]⁺ found 396.3.

### Example 19—Preparation of: N-(5-(5-((1R,2S)-2-fluorocyclopropyl)-1,2,4-oxadiazol-3-yl)-2-methylphenyl)-7-(((3-hydroxyoxetan-3-yl)methoxy)methyl)imidazo[1,2-a]pyridine-3-carboxamide (I-490)

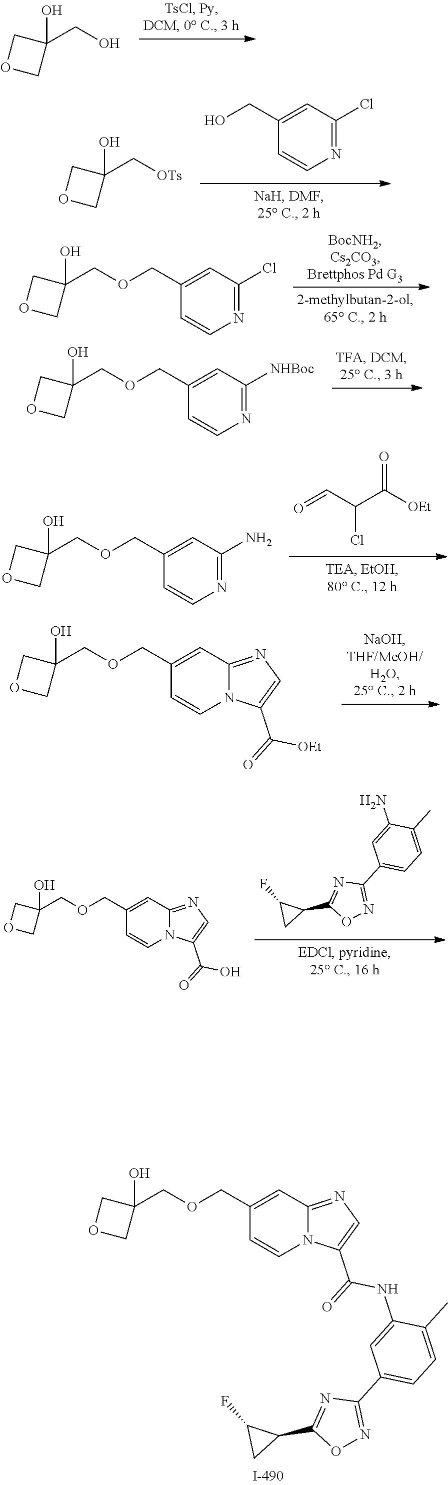

Step 1: To a solution of 3-(hydroxymethyl)oxetan-3-ol (1.7 g, 16.33 mmol) in DCM (20 mL) and pyridine (20 mL) was added TosCl (4.05 g, 21.23 mmol) at 0° C. under nitrogen. The mixture was stirred at 0° C. for 3 hours. The reaction mixture was concentrated under reduced pressure to remove DCM. The residue was diluted with H₂O 20 mL and extracted with ethyl acetate (50 mL×3). The combined organic layers were washed with brine (30 mL×2), dried over Na₂SO₄, filtered and concentrated under reduced pressure to give a residue. The residue was purified by column chromatography (SiO₂, petroleum ether:ethyl acetate=10:1 to 1:1) to get (3-hydroxyoxetan-3-yl)methyl 4-methylbenzenesulfonate (1.85 g, 7.16 mmol, 43.86% yield) as a white solid. MS (ESI): m/z for C₁₁H₁₄O₅S. ¹H NMR (400 MHz, CD₃OD) δ: 7.83 (d, J=8.4 Hz, 2H), 7.40 (d, J=8.4 Hz, 2H), 4.61 (d, J=7.6 Hz, 2H), 4.41 (d, J=7.6 Hz, 2H), 4.32 (s, 2H), 2.94 (s, 1H), 2.48 (s, 3H).

Step 2: To a solution of (2-chloropyridin-4-yl)methanol (0.95 g, 6.62 mmol) in DMF (20 mL) was added NaH (397 mg, 9.93 mmol, 60% purity) at 0° C. under N₂, then the reaction mixture was stirred at 0° C. for 0.5 hour, then (3-hydroxyoxetan-3-yl)methyl 4-methylbenzenesulfonate (1.71 g, 6.62 mmol) was added, the reaction mixture was stirred at 25° C. for 2 hours. The reaction mixture was quenched with H₂O 50 mL and extracted with ethyl acetate (50 mL×3). The combined organic layers were washed with brine (25 mL×2), dried over Na₂SO₄, filtered and concentrated under reduced pressure to give a residue. The residue was purified by column chromatography (SiO₂, petroleum ether:ethyl acetate=5:1 to 0:1) to get compound tert-butyl 3-(((2-chloropyridin-4-yl)methoxy)methyl)oxetan-3-ol (350 mg, 1.48 mmol, 22.34% yield) as a white solid. MS (ESI): m/z for C₁₈H₂₇FN₂O₅[M+H]⁺ calcd. 230.0 [MH]⁺ found 229.05. ¹H NMR (400 MHz, CD₃OD) δ: 8.31 (d, J=4.8 Hz, 1H), 7.51 (s, 1H), 7.38 (d, J=5.2 Hz, 1H), 4.69 (s, 2H), 4.63-4.54 (m, 4H), 3.74 (s, 2H), 3.05-2.84 (m, 1H).

Step 3: To a solution of 3-(((2-chloropyridin-4-yl)methoxy)methyl)oxetan-3-ol (350 mg, 1.52 mmol) in 2-methylbutan-2-ol (5 mL) was added Cs₂CO₃ (1.49 g, 4.57 mmol) and NH₂Boc (536 mg, 4.57 mmol), BrettPhos Pd G₃ (138 mg, 152 mol). The mixture was stirred at 65° C. for 2 hours under N₂ atmosphere. The reaction mixture was diluted with H₂O 20 mL and extracted with ethyl acetate (40 mL×3). The combined organic layers were washed with brine (25 mL×2), dried over Na₂SO₄, filtered and concentrated under reduced pressure to give a residue. The residue was purified by column chromatography (SiO₂, petroleum ether:ethyl acetate=5:1 to 0:1) to get compound tert-butyl (4-(((3-hydroxyoxetan-3-yl)methoxy)methyl)pyridin-2-yl)carbamate (380 mg, 1.19 mmol, 77.93% yield) as a white solid. MS (ESI): m/z for C₁₅H₂₂N₂O₅ [M+H]⁺ calcd. 311.2 [MH]⁺ found 310.15

Step 4: To a solution of tert-butyl (4-(((3-hydroxyoxetan-3-yl)methoxy)methyl)pyridin-2-yl)carbamate (300 mg, 967 μmol) in DCM (3 mL) was added TFA (921 mg, 8.08 mmol, 0.6 mL). The mixture was stirred at 25° C. for 3 hours. The reaction mixture was concentrated under reduced pressure to get 3-(((2-aminopyridin-4-yl)methoxy)methyl)oxetan-3-ol (205 mg, crude) as a yellow solid. MS (ESI): m/z for C₁₀H₁₄N₂O₃ [M+H]⁺ calcd. 211.1, [MH]⁺ found 210.10.

Step 5: To a solution of 3-(((2-aminopyridin-4-yl)methoxy)methyl)oxetan-3-ol (205 mg, 975 μmol) in EtOH (3 mL) was added triethylamine (296 mg, 2.93 mmol, 407 L) and ethyl 2-chloro-3-oxo-propanoate (294 mg, 1.95 mmol). The mixture was stirred at 80° C. for 12 hours. The reaction mixture was diluted with H₂O 20 mL and extracted with ethyl acetate (50 mL×3). The combined organic layers were washed with brine (25 mL×2), dried over Na₂SO₄, filtered and concentrated under reduced pressure to give a residue. The residue was purified by column chromatography (SiO₂, petroleum ether:ethyl acetate=3:1 to 0:1) to get ethyl 7-(((3-hydroxyoxetan-3-yl)methoxy)methyl)imidazo[1,2-a]pyridine-3-carboxylate (160 mg, 428.32 mol, 43.92% yield) as a white solid. MS (ESI): m/z for C₁₅H₁₈N₂O₅ [M+H]⁺ calcd. 307.2, [MH]⁺ found 306.12. ¹H NMR (400 MHz, CD₃OD) δ:9.30 (d, J=6.8 Hz, 1H), 8.24 (s, 1H), 7.73 (s, 1H), 7.24 (d, J=7.2 Hz, 1H), 4.77 (s, 2H), 4.66-4.53 (m, 4H), 4.48-4.38 (m, 2H), 3.77 (s, 2H), 1.42 (t, J=7.2 Hz, 3H).

Step 6: To a solution of ethyl 7-(((3-hydroxyoxetan-3-yl)methoxy)methyl)imidazo[1,2-a]pyridine-3-carboxylate (110 mg, 359 μmol) in THE (1 mL), MeOH (1 mL), H₂O (1 mL) was added NaOH (29 mg, 718 μmol), the mixture was stirred at 25° C. for 1 hour. The reaction mixture was concentrated under reduced pressure to remove THF and MeOH, then was acid with citric acid (0.7 eq), then was concentrated under reduced pressure. The solid was dissolved in EtOH (30 mL), the mixture was stirred at 25° C. for 1 hour, then was filtered, the filtrate was concentrated under reduced pressure to get 7-(((3-hydroxyoxetan-3-yl)methoxy)methyl)imidazo[1,2-a]pyridine-3-carboxylic acid (60 mg, crude) as a white solid. MS (ESI): m/z for C₁₃H₁₄N₂O₅ [M+H]⁺ calcd. 278.8, [MH]⁺ found 278.09.

Step 7: To a solution of 7-(((3-hydroxyoxetan-3-yl)methoxy)methyl)imidazo[1,2-a]pyridine-3-carboxylic acid (56 mg, 201 μmol) in pyridine (1.5 mL) was added EDCI (62 mg, 322 μmol), the mixture was stirred at 25° C. for 2 hours, then 5-(5-((1R,2S)-2-fluorocyclopropyl)-1,2,4-oxadiazol-3-yl)-2-methylaniline (47 mg, 201 μmol) was added, the reaction mixture was stirred at 25° C. for 10 hours. The reaction mixture was diluted with H₂O 20 mL and extracted with ethyl acetate (50 mL×2). The combined organic layers were washed with brine (25 mL×2), dried over Na₂SO₄, filtered and concentrated under reduced pressure to give a residue. The residue was purified by prep-HPLC (FA condition; column: YMC-Actus Triart C18 150×30 mm×7 um; mobile phase: [water(FA)-ACN]; gradient:25%-55% B over 10 min) to give compound N-(5-(5-((1R,2S)-2-fluorocyclopropyl)-1,2,4-oxadiazol-3-yl)-2-methylphenyl)-7-(((3-hydroxyoxetan-3-yl)methoxy)methyl)imidazo[1,2-a]pyridine-3-carboxamide (I-490) (26.8 mg, 53.62 μmol, 26.64% yield) as a yellow solid. MS (ESI): m/z for C₂₅H₂₄FN₅O₅[M+H]⁺ calcd. 494.2, [M+H]⁺ found. 494.2. ¹H NMR (400 MHz, CD₃OD) δ: 9.46 (d, J=7.2 Hz, 1H), 8.45 (s, 1H), 8.07 (d, J=1.6 Hz, 1H), 7.85 (dd, J=1.6, 8.0 Hz, 1H), 7.74 (s, 1H), 7.45 (d, J=8.0 Hz, 1H), 7.18 (dd, J=1.6, 7.2 Hz, 1H), 5.26-5.00 (m, 1H), 4.78 (s, 2H), 4.66-4.54 (m, 4H), 3.77 (s, 2H), 2.92-2.77 (m, 1H), 2.40 (s, 3H), 1.95-1.81 (m, 1H), 1.66-1.54 (m, 1H).

### Example 20—Preparation of: N-(5-(5-((1R,2S)-2-fluorocyclopropyl)-1,2,4-oxadiazol-3-yl)-2-methylphenyl)-5-isopropoxypyrazolo[1,5-a]pyridine-3-carboxamide (I-493)

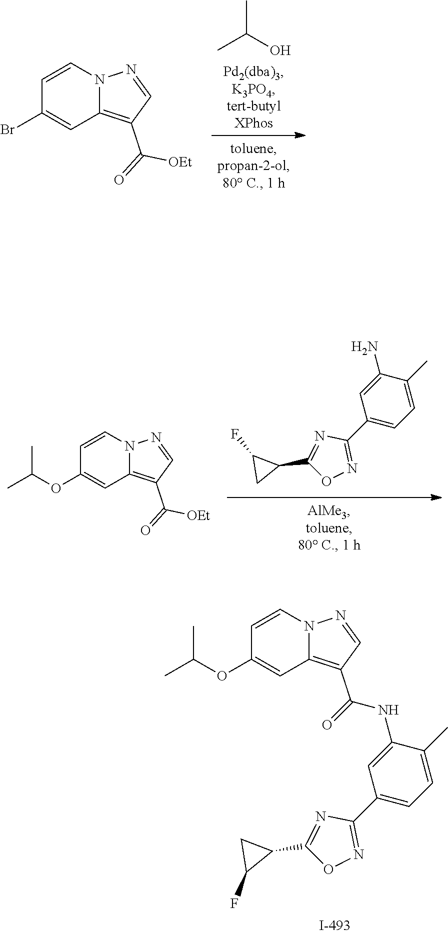

Step 1: To a solution of ethyl 5-bromopyrazolo[1,5-a]pyridine-3-carboxylate (600 mg, 1.0 eq) in propan-2-ol (6 mL) and toluene (12 mL) was added K₃PO₄ (946 mg, 2.0 eq), ditert-butyl-[2-(2,4,6-triisopropylphenyl)phenyl]phosphane (189 mg, 0.2 eq), Pd₂(dba)₃ (204 mg, 0.1 eq). The mixture was stirred at 80° C. for 1 hour. The reaction mixture was partitioned between H₂O 50 mL and ethyl acetate (70 mL×3). The organic phase was separated, washed with brine 150 mL (50 mL×3), dried over Na₂SO₄, filtered and concentrated under reduced pressure to give a residue. The residue was purified by column chromatography (SiO₂, petroleum ether/ethyl acetate=10/1 to 7/1) to give ethyl 5-isopropoxypyrazolo[1,5-a]pyridine-3-carboxylate (450 mg, 76% yield) as an orange oil. ¹H NMR (400 MHz, DMSO-d₆) δ=8.75-8.65 (m, 1H), 8.33-8.26 (m, 1H), 7.32 (d, J=2.8 Hz, 1H), 6.82-6.72 (m, 1H), 4.83-4.70 (m, 1H), 4.33-4.21 (m, 2H), 1.39-1.27 (m, 9H). MS (ESI): m/z for C₁₃H₁₆N₂O₃ [M+H]+ calcd. 249.3 [M+H]+ found 249.3

Step 2: To a solution of ethyl 5-isopropoxypyrazolo[1,5-a]pyridine-3-carboxylate (200 mg, 1.0 eq) and 5-[5-[(1R,2S)-2-fluorocyclopropyl]-1,2,4-oxadiazol-3-yl]-2-methyl-aniline (188 mg, 1.0 eq) in toluene (2 mL) was added AlMe₃ (2 M, 2.5 eq). The mixture was stirred at 80° C. for 1 hour. The reaction mixture was quenched with 20 ml ice water, filtered and filtrate was extracted with DCM (150 mL), dried over Na₂SO₄, filtered and concentrated in vacuum. The residue was purified by prep-HPLC (column: YMC Triart C18 150*25 mm*5 um; mobile phase: [water(FA)-ACN]; gradient:55%-85% B over 10 min) to give N-(5-(5-((1R,2S)-2-fluorocyclopropyl)-1,2,4-oxadiazol-3-yl)-2-methylphenyl)-5-isopropoxypyrazolo[1,5-a]pyridine-3-carboxamide (I-493) (174 mg, 44.3% yield) as a white solid. ¹H NMR (400 MHz, DMSO-d6) δ=9.57 (s, 1H), 8.72-8.62 (m, 2H), 8.06 (d, J=1.2 Hz, 1H), 7.77-7.69 (m, 1H), 7.54 (d, J=2.8 Hz, 1H), 7.45 (d, J=8.0 Hz, 1H), 6.77-6.70 (m, 1H), 5.40-5.18 (m, 1H), 4.80-4.65 (m, 1H), 3.11-2.98 (m, 1H), 2.36 (s, 3H), 2.02-1.87 (m, 1H), 1.66-1.53 (m, 1H), 1.35 (d, J=6.0 Hz, 6H). MS (ESI): m/z for C₂₅H₂₃FN₆O₃[M+H]+ calcd. 436.3 [M+H]+ found 436.4

### Example 21—Preparation of 5-(difluoromethyl)-N-(5-(5-((1R,2S)-2-fluorocyclopropyl)-1,2,4-oxadiazol-3-yl)-2-methylphenyl)pyrazolo[1,5-a]pyridine-3-carboxamide (I-494)

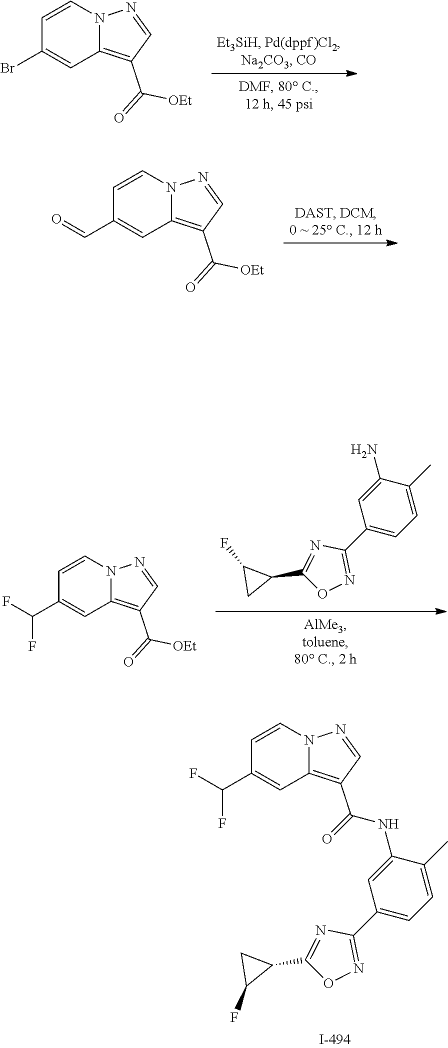 [SMILES: CCOC(=O)c1cnn2ccc(Br)cc12 ; CCOC(=O)c1cnn2ccc(C=O)cc12 ; CCOC(=O)c1cnn2ccc(C(F)F)cc12 ; Cc1ccc(-c2noc([C@H]3C[C@@H]3F)n2)cc1N ; Cc1ccc(-c2noc([C@H]3C[C@@H]3F)n2)cc1NC(=O)c1cnn2ccc(C(F)F)cc12]

Step 1: To a solution of ethyl 5-bromopyrazolo[1,5-a]pyridine-3-carboxylate (300 mg, 1.0 eq) in DMF (3 mL) was added Et₃SiH (259 mg, 2.0 eq), Pd(dppf)Cl₂ (81 mg, 0.1 eq) and Na₂CO₃ (118 mg, 1.0 eq). The mixture was stirred at 80° C. for 12 hours under CO (45 Psi) atmosphere. The reaction mixture was diluted with water 60 mL and extracted with ethyl acetate (100 mL×3). The combined organic layers were washed with brine 300 mL (50 mL×6), dried over Na₂SO₄, filtered and concentrated under reduced pressure to give a residue. The residue was purified by column chromatography (SiO₂, petroleum ether/ethyl acetate=5/1) to give ethyl 5-formylpyrazolo[1,5-a]pyridine-3-carboxylate (115 mg, 86% purity) as a white solid. ¹H NMR (400 MHz, DMSO-d₆) δ=10.15 (s, 1H), 8.98 (d, J=7.2 Hz, 1H), 8.67 (d, J=0.4 Hz, 1H), 8.60 (s, 1H), 7.45-7.41 (m, 1H), 4.39-4.33 (m, 2H), 1.37 (t, J=7.2 Hz, 3H). MS (ESI): m/z for C₁₁H₁₀N₂O₃ [M+H]⁺ calcd. 219.07, [M+H]⁺ found 218.9.

Step 2: To a solution of ethyl 5-formylpyrazolo[1,5-a]pyridine-3-carboxylate (100 mg, 1.0 eq) in DCM (5 mL) was added DAST (369 mg, 5.0 eq) at 0° C., the mixture was stirred at 25° C. for 12 hours. The reaction mixture was quenched by addition NH₄Cl 1 mL at 0° C., and then diluted with water 15 mL and extracted with ethyl acetate (20 mL×3). The combined organic layers were washed with brine (10 mL×3), dried over Na₂SO₄, filtered and concentrated under reduced pressure to give a residue. The residue was purified by column chromatography (SiO₂, petroleum ether/ethyl acetate=19/1) to give ethyl 5-(difluoromethyl)pyrazolo[1,5-a]pyridine-3-carboxylate (85 mg, 81% purity) as a white solid. ¹H NMR (400 MHz, DMSO-d₆) δ=9.01 (d, J=5.2 Hz, 1H), 8.60-8.53 (m, 1H), 8.30 (s, 1H), 7.41-7.10 (m, 2H), 4.38-4.31 (m, 2H), 1.39-1.32 (m, 3H). MS (ESI): m/z for C₁₁H₁₀F₂N₂O₂[M+H]⁺ calcd. 241.07, [M+H]⁺ found 240.8.

Step 3: To a solution of ethyl 5-(difluoromethyl)pyrazolo[1,5-a]pyridine-3-carboxylate (75 mg, 1.0 eq) and 5-[5-[(1R,2S)-2-fluorocyclopropyl]-1,2,4-oxadiazol-3-yl]-2-methyl-aniline (80 mg, 1.1 eq) in the toluene (1 mL) was added AlMe₃ (2 M, 2.5 eq). The mixture was stirred at 80° C. for 2 hours under N₂ atmosphere. The reaction mixture was quenched by addition NH₄Cl solution 1 mL at 0° C. and filtered, the filtrate was diluted with water 50 mL and extracted with ethyl acetate (80 mL×3). The combined organic layers were washed with brine (60 mL×3), dried over Na₂SO₄, filtered and concentrated under reduced pressure to give a residue. The residue was purified by prep-HPLC (FA condition; column: Phenomenex luna C18 150*25 mm*10 um; mobile phase: [water(FA)-ACN]; gradient: 48%-78% B over 9 min) to give 5-(difluoromethyl)-N-(5-(5-((1R,2S)-2-fluorocyclopropyl)-1,2,4-oxadiazol-3-yl)-2-methylphenyl)pyrazolo[1,5-a]pyridine-3-carboxamide (I-494) (61 mg, 99.7% purity, FA) as a white solid. ¹H NMR (400 MHz, DMSO-d₆) δ=9.90 (s, 1H), 8.99 (d, J=7.2 Hz, 1H), 8.87 (s, 1H), 8.48 (s, 1H), 8.05 (s, 1H), 7.78-7.74 (m, 1H), 7.47 (d, J=8.0 Hz, 1H), 7.39-7.09 (m, 2H), 5.39-5.19 (m, 1H), 3.11-3.00 (m, 1H), 2.36 (s, 3H), 2.01-1.88 (m, 1H), 1.64-1.54 (m, 1H). MS (ESI): m/z for C₂₁H₁₆F₃N₅O₂[M+H]⁺ calcd. 428.13, [MH]⁺ found 428.2.

### Example 22—Preparation of 5-fluoro-N-[5-[5-[(1R,2S)-2-fluorocyclopropyl]-1,2,4-oxadiazol-3-yl]-2-methyl phenyl]pyrazolo[1,5-a]pyridine-3-carboxamide (I-496)

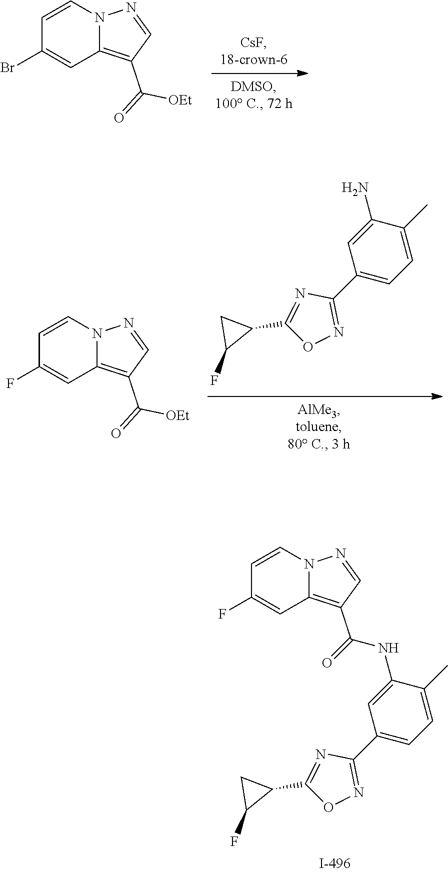 [SMILES: CCOC(=O)c1cnn2ccc(Br)cc12 ; CCOC(=O)c1cnn2ccc(F)cc12 ; Cc1ccc(-c2noc([C@H]3C[C@@H]3F)n2)cc1N ; Cc1ccc(-c2noc([C@H]3C[C@@H]3F)n2)cc1NC(=O)c1cnn2ccc(F)cc12]

Step 1: A mixture of ethyl 5-bromopyrazolo[1,5-a]pyridine-3-carboxylate (0.3 g, 1.0 eq) 18-crown-6 (29 mg, 0.1 eq) and CsF (508 mg, 3.0 eq) in DMSO (6 mL) was stirred at 100° C. for 72 hours under nitrogen. The reaction mixture was filtered. The filtrate was purified by reversed-phase HPLC (0.1% FA condition) to give ethyl 5-fluoropyrazolo[1,5-a]pyridine-3-carboxylate (130 mg) as yellow solid. ¹H NMR (400 MHz, DMSO-d₆) δ 8.99 (dd, J=5.2, 7.6 Hz, 1H), 8.47 (s, 1H), 7.72 (dd, J=2.8, 8.8 Hz, 1H), 7.21 (m, 1H), 4.30 (q, J=7.2 Hz, 2H), 1.33 (t, J=7.2 Hz, 3H). MS (ESI): m/z for C₁₃H₁₆N₂O₃ [M+H]+ calcd. 209.1 [M+H]+ found 209.1

Step 2: To a solution of ethyl 5-fluoropyrazolo[1,5-a]pyridine-3-carboxylate (110 mg, 1.0 eq) and 5-[5-[(1R,2S)-2-fluorocyclopropyl]-1,2,4-oxadiazol-3-yl]-2-methyl-aniline (123 mg, 1.0 eq) in toluene (3 mL) was added AlMe₃ (2 M, 2.5 eq) at 0° C., the reaction mixture was stirred at 80° C. for 3 hours. The reaction mixture was quenched with NH₄Cl sat. (1 mL), extracted with DCM (30 mL), washed with brine (10 mL×2), the organic layer was dried over Na₂SO₄, filtered and concentrated in vacuum. The residue was purified by prep-HPLC (column: Phenomenex luna C18 150*25 mm*10 um; mobile phase: [water(FA)-ACN]; gradient: 45%-75% B over 10 min) to give 5-fluoro-N-[5-[5-[(1R,2S)-2-fluorocyclopropyl]-1,2,4-oxadiazol-3-yl]-2-methyl phenyl]pyrazolo[1,5-a]pyridine-3-carboxamide (I-496 (88.67 mg, 99% purity, FA) as white solid. ¹H NMR (400 MHz, DMSO-d₆) δ 9.76 (s, 1H), 8.96 (dd, J=5.2, 7.6 Hz, 1H), 8.80 (s, 1H), 8.03 (d, J=1.6 Hz, 1H), 7.88 (dd, J=2.8, 9.2 Hz, 1H), 7.75 (dd, J=1.6, 8.0 Hz, 1H), 7.46 (d, J=8.0 Hz, 1H), 7.17 (dt, J=2.8, 7.6 Hz, 1H), 5.42-5.16 (m, 1H), 3.12-2.98 (m, 1H), 2.34 (s, 3H), 2.03-1.86 (m, 1H), 1.58 (dd, J=6.4, 13.1 Hz, 1H). MS (ESI): m/z for C₂₁H₁₇N₅O₄F₂ [M+H]⁺ calcd. 396.1, [M+H]⁺ found 396.1.

### Example 23—Preparation of 6-fluoro-N-[5-[5-[(1R,2S)-2-fluorocyclopropyl]-1,2,4-oxadiazol-3-yl]-2-methyl-phenyl]-7-[(2-hydroxy-2-methyl-propoxy)methyl]imidazo[1,2-a]pyridine-3-carboxamide (I-498)

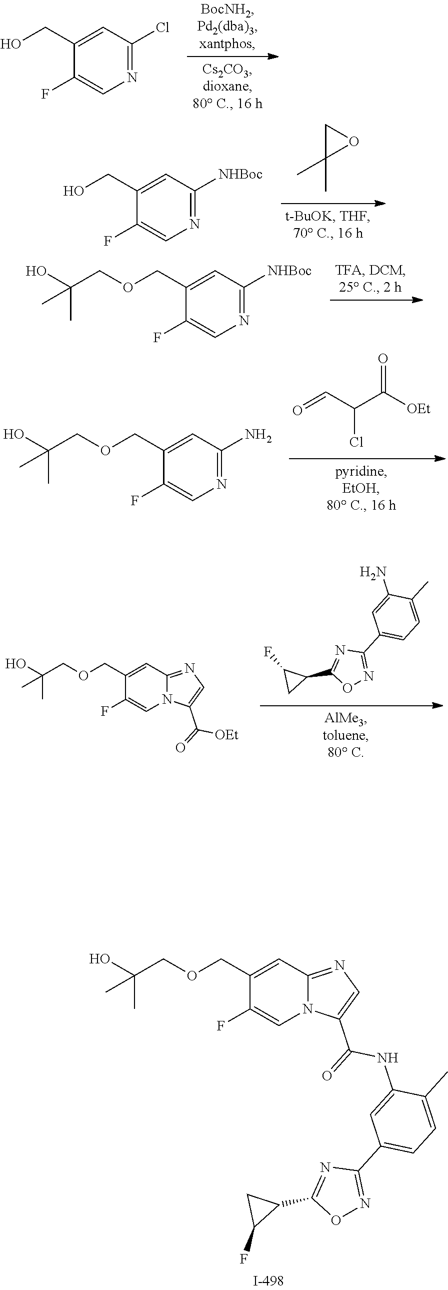

Step 1: To a solution of (2-chloro-5-fluoro-4-pyridyl)methanol (5.00 g, 30.9 mmol) and tert-butyl carbamate (10.8 g, 92.8 mmol) in 2-methylbutan-2-ol (100 mL) was added Cs₂CO₃ (20.1 g, 61.9 mmol) and dicyclohexyl-[3,6-dimethoxy-2-(2,4,6-triisopropylphenyl)phenyl]phosphane; methanesulfonate; [2-[2-(methylamino)phenyl] phenyl]palladium(1+) (2.85 g, 3.09 mmol). The mixture was stirred at 80° C. for 2 hr under N₂ protected. On completion, then diluted with water 50 mL and extracted with ethyl acetate 150 mL (50 mL×3). The combined organic layers were washed with brine 50 mL, dried over Na₂SO₄, filtered and concentrated under reduced pressure to give a residue. The crude product was purified by reversed-phase HPLC (0.1% FA condition) to give tert-butyl N-[5-fluoro-4-(hydroxymethyl)-2-pyridyl]carbamate (7 g, 84.03% yield, 90% purity) as white solid. ¹H NMR: 400 MHz, DMSO-d6 δ=9.79 (s, 1H), 8.12 (d, J=1.0 Hz, 1H), 7.99 (d, J=5.6 Hz, 1H), 5.53 (t, J=6.0 Hz, 1H), 4.58 (d, J=5.6 Hz, 2H), 1.48 (s, 9H).

Step 2: To a 100 ml three-necked flask of tert-butyl N-[5-fluoro-4-(hydroxymethyl)-2-pyridyl]carbamate (2.00 g, 8.26 mmol) in dry THF (40 mL) at 0° C. under N₂ protect, then t-BuOK (1 M, 12.38 mL) was added to the mixture, the mixture was stirred at 0° C. for 30 min, then 2,2-dimethyloxirane (1.79 g, 24.7 mmol, 2.20 mL) was added to the mixture at 0° C., the mixture was stirred at 70° C. for 16 hr. On completion, the reaction mixture was quenched by addition water 10 mL at 0° C., and then extracted with EA 60 mL (20 mL×3). The combined organic layers were washed with brine 30 mL, dried over Na₂SO₄, filtered and concentrated under reduced pressure to give a residue. The crude product was purified by reversed-phase HPLC (0.1% FA condition) to give tert-butyl N-[5-fluoro-4-[(2-hydroxy-2-methyl-propoxy)methyl]-2-pyridyl]carbamate (1.2 g, 41.61% yield, 90% purity) as white solid. MS (ESI): m/z [M+H]⁺ calcd. 315.2.

Step 3: To a solution of tert-butyl N-[5-fluoro-4-[(2-hydroxy-2-methyl-propoxy)methyl]-2-pyridyl]carbamate (300 mg, 0.1 mol) in DCM (10 mL) was added TFA (3.07 g, 26.9 mmol, 2.00 mL). The mixture was stirred at 25° C. for 2 hr. TLC (petroleum ether:ethyl acetate 3:1) indicated Reactant 1 was consumed completely and one new spot formed. The reaction was clean according to TLC. The mixture was concentrated in vacuum to give 1-[(2-amino-5-fluoro-4-pyridyl)methoxy]-2-methyl-propan-2-ol: (210 mg, crude) as yellow oil for next step directly without further purification.

Step 4: To a solution of 1-[(2-amino-5-fluoro-4-pyridyl)methoxy]-2-methyl-propan-2-ol (210 mg, 980 μmol) and ethyl 2-chloro-3-oxo-propanoate (177 mg, 1.18 mmol) in EtOH (10 mL) was added triethylamine (297 mg, 2.94 mmol, 409 μL). The mixture was stirred at 80° C. for 12 hr. On completion, the mixture was concentrated in vacuo. The crude product was purified by reversed-phase HPLC (0.1% FA condition) to give 6-fluoro-7-[(2-hydroxy-2-methyl-propoxy)methyl]imidazo[1,2-a]pyridine-3-carboxylate (100 mg, 31% yield, 95% purity) as yellow oil. MS (ESI): m/z [M+H]⁺ calcd. 311.0.

Step 5: To a solution of ethyl 6-fluoro-7-[(2-hydroxy-2-methyl-propoxy)methyl]imidazo[1,2-a]pyridine-3-carboxylate (100 mg, 322 μmol) and 5-[5-[(1R,2S)-2-fluorocyclopropyl]-1,2,4-oxadiazol-3-yl]-2-methyl-aniline (82.6 mg, 354 μmol) in toluene (5 mL) was added AlMe₃ (2 M, 402 μL). The mixture was stirred at 80° C. for 2 hr. On completion, the reaction mixture was quenched by addition H₂O 0.5 mL at 25° C., and then extracted with ethyl acetate (20 mL×3). The combined organic layers were washed with brine 20 mL, dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; gradient: 40%-70% B over 9 min) to give 6-fluoro-N-[5-[5-[(1R,2S)-2-fluorocyclopropyl]-1,2,4-oxadiazol-3-yl]-2-methyl-phenyl]-7-[(2-hydroxy-2-methyl-propoxy)methyl]imidazo[1,2-a]pyridine-3-carboxamide (I-498) (95 mg, 56% yield, 95% purity) as yellow solid. ¹H NMR 400 MHz, CHLOROFORM-d δ=9.53 (d, J=5.6 Hz, 1H), 8.50 (s, 1H), 8.20 (s, 1H), 7.87-7.78 (m, 2H), 7.63 (s, 1H), 7.38 (d, J=7.9 Hz, 1H), 5.19-4.95 (m, 1H), 4.76 (s, 2H), 3.48 (s, 2H), 2.80-2.67 (m, 1H), 2.42 (s, 3H), 1.90-1.80 (m, 1H), 1.67-1.59 (m, 1H), 1.30 (s, 6H). MS (ESI): m/z [M+H]+ calcd. 498.2.

### Example 24—Preparation of 7-((3,3-difluoro-2-hydroxypropoxy)methyl)-N-(5-(5-((1R,2S)-2-fluorocyclopropyl)-1,2,4-oxadiazol-3-yl)-2-methylphenyl)imidazo[1,2-a]pyridine-3-carboxamide (I-499), 7-(((R)-3,3-difluoro-2-hydroxypropoxy)methyl)-N-(5-(5-((1R,2S)-2-fluorocyclopropyl)-1,2,4-oxadiazol-3-yl)-2-methylphenyl)imidazo[1,2-a]pyridine-3-carboxamide (I-500), and 7-(((S)-3,3-difluoro-2-hydroxypropoxy)methyl)-N-(5-(5-((1R,2S)-2-fluorocyclopropyl)-1,2,4-oxadiazol-3-yl)-2-methylphenyl)imidazo[1,2-a]pyridine-3-carboxamide (I-501)

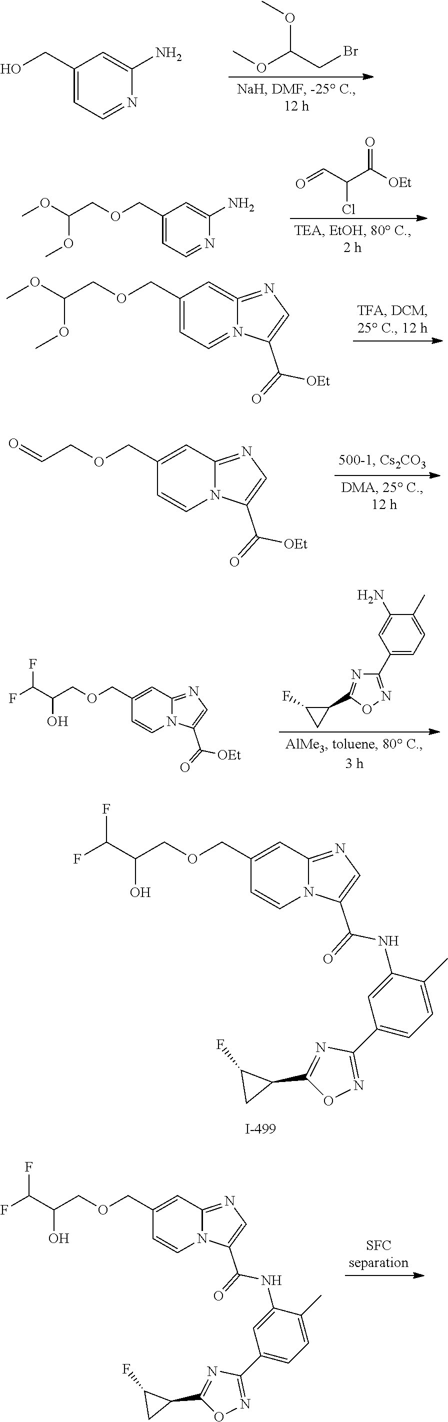

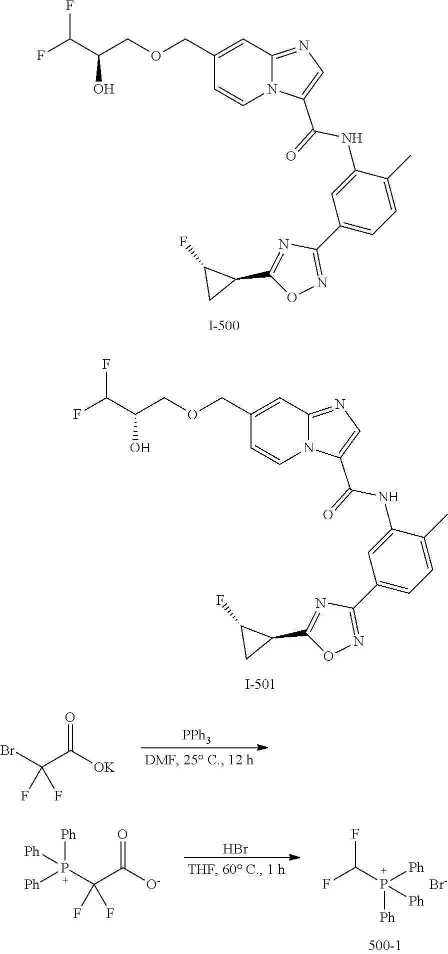

Step 1: To a solution of (2-amino-4-pyridyl)methanol (3 g, 1.0 eq) in DMF (60 mL) was added NaH (1.45 g, 60% purity, 1.5 eq) slowly at 0° C. under nitrogen. The mixture was stirred at 0° C. for 0.5 hour. To the mixture was added 2-bromo-1,1-dimethoxy-ethane (12.25 g, 8.51 mL, 3 eq) slowly. The mixture was stirred at 25° C. for 12 hours. To the mixture was added 50 mL of NH₄Cl (aq.) to quench the reaction mixture. The mixture was extracted with ethyl acetate (30 mL×3). The organic layer was washed with brine (30 mL×2), dried over Na₂SO₄, filtered and concentrated to give title 4-((2,2-dimethoxyethoxy)methyl)pyridin-2-amine (6 g, crude) as yellow oil. MS (ESI): m/z for C₁₀H₁₆N₂O₃ [M+H]⁺ calcd. 213.1, [M+H]⁺ found 213.1.

Step 2: To a solution of 4-(2,2-dimethoxyethoxymethyl)pyridin-2-amine (6 g, 1 eq) and ethyl 2-chloro-3-oxopropanoate (4.26 g, 1 eq) in EtOH (50 mL) was added triethylamine (2.86 g, 3.93 mL, 1 eq). The mixture was stirred at 80° C. for 1 hour. The mixture was concentrated to give a residue. The residue was purified by column chromatography (SiO2, petroleum ether/ethyl acetate=1/1) to give ethyl 7-((2,2-dimethoxyethoxy)methyl)imidazo[1,2-a]pyridine-3-carboxylate (2.6 g, 98% purity) as yellow oil. MS (ESI): m/z for C₁₅H₂₀N₂O₅ [M+H]⁺ calcd. 309.2, [M+H]⁺ found 309.3. ¹H NMR (400 MHz, CHLOROFORM-d) δ=9.29-9.27 (d, J=8.0 Hz, 1H), 8.30 (s, 1H), 7.71 (s, 1H), 7.06 (d, J=8.0 Hz, 1H), 4.70 (s, 2H), 4.60 (t, J=5.2 Hz, 1H), 4.46-4.41 (m, 2H), 3.60 (d, J=5.2 Hz, 2H), 3.44 (s, 6H), 1.46-1.43 (m, 3H).

Step 3: To a solution of ethyl 7-(2,2-dimethoxyethoxymethyl)imidazo[1,2-a]pyridine-3-carboxylate (1.2 g, 1 eq) in DCM (10 mL) was added TFA (4.61 g, 3 mL, 10.38 eq). The mixture was stirred at 40° C. for 2 hours. The mixture was concentrated to give ethyl 7-((2-oxoethoxy)methyl)imidazo[1,2-a]pyridine-3-carboxylate (1.1 g, crude) as yellow oil. MS (ESI): m/z for C13H14N2O4 [M+H]⁺ calcd. 263.1, [M+18]⁺ found 281.1. ¹H NMR (400 MHz, DMSO-d₆) δ=9.64 (s, 1H), 9.23-9.21 (d, J=7.2 Hz, 1H), 8.61 (s, 1H), 7.43 (d, J=7.2 Hz, 1H), 4.78 (s, 2H), 4.43-4.39 (m, 4H), 1.29-1.26 (m, 3H).

Step 4: To a mixture of ethyl 7-(2-oxoethoxymethyl)imidazo[1,2-a]pyridine-3-carboxylate (1100 mg, 1 eq) and Cs₂CO₃ (8.20 g, 6 eq) in DMA (10 mL) was added bromo-(difluoromethyl)-triphenyl-phosphane (3.30 g, 2 eq). The mixture was stirred at 25° C. for 12 hours. The mixture was added 20 mL of water and extracted with DCM (30 mL×3). The organic layer was washed with brine 30 mL, dried over Na₂SO₄, filtered and concentrated to give a residue. The residue was purified by reversed phase flash (FA condition) to give ethyl 7-((3,3-difluoro-2-hydroxypropoxy)methyl)imidazo[1,2-a]pyridine-3-carboxylate (180 mg, 97% purity) as yellow solid. MS (ESI): m/z for: C14H16F2N2O4 [M+H]⁺ calcd 315.2, [M+H]⁺ found 315.2. ¹H NMR (400 MHz, DMSO-d₆) δ=9.19 (d, J=7.2 Hz, 1H), 8.28 (s, 1H), 7.76 (s, 1H), 7.25-7.16 (m, 1H), 6.15-5.84 (m, 1H), 5.81 (s, 1H), 4.67 (s, 2H), 4.42-4.31 (m, 2H), 3.97-3.82 (m, 1H), 3.68-3.53 (m, 2H), 1.35 (t, J=7.2 Hz, 3H)

Step 5: To a mixture of potassium; 2-bromo-2,2-difluoro-acetate (10.3 g, 1 eq) and triphenylphosphane (12.68 g, 1 eq) in DMF (100 mL) was stirred at 25° C. for 12 h. Most of the white solid was separated out. The mixture was filtered and the solid was washed with ethanol (45 mL×2), petroleum ether (50 mL×3). The solid was dried with high vacuum to give 2,2-difluoro-2-(triphenylphosphonio)acetate (14 g, crude) as white solid.

Step 6: To a mixture of 2,2-difluoro-2-triphenylphosphaniumyl-acetate (7.12 g, 1.0 eq) and HBr (4.47 g, 3 mL, 40% purity, 1.1 eq) in THF (20 mL). The mixture was stirred at 75° C. for 1 h. The mixture was cooled it to 25° C. and concentrated to remove most of solvent. The residue was added 40 mL of water, and extracted with DCM (30 mL×3), dried over Na₂SO₄, filtered and concentrated to give (difluoromethyl)triphenylphosphonium (500-1) (6 g, crude) as white solid. MS (ESI): m/z for: C19H16F2BrP [M+H]⁺ calcd. 393.21, [M+H]⁺ found 313.2 M-Br. Ref: Organic Letters, 2014, vol. 16, #23, p. 6256 6259

Step 7: To a solution of ethyl 7-[(3,3-difluoro-2-hydroxy-propoxy)methyl]imidazo[1,2-a]pyridine-3-carboxylate (150 mg, 1 eq) and 5-[5-[(1R,2S)-2-fluorocyclopropyl]-1,2,4-oxadiazol-3-yl]-2-methyl-aniline (111 mg, 1 eq) in toluene (3 mL) was added AlMe₃ (2 M, 2.5 eq). The mixture was heated to 80° C. and stirred at 80° C. for 2 hours. The mixture was poured into 10 mL of water and extracted with dichloromethane (20 mL×3). The organic layer was washed with brine, dried over Na₂SO₄, filtered and concentrated to give a residue. The residue was purified by prep-HPLC (column: Phenomenex luna C18 150*25 mm*10 um; mobile phase: [water(FA)-ACN]; gradient: 30%-60% B over 7 min) to 7-((3,3-difluoro-2-hydroxypropoxy)methyl)-N-(5-(5-((1R,2S)-2-fluorocyclopropyl)-1,2,4-oxadiazol-3-yl)-2-methylphenyl)imidazo[1,2-a]pyridine-3-carboxamide (I-499) (99.8 mg, 95% purity, FA) as yellow gum. MS (ESI): m/z for: C24H22F3N5O4 [M+H]⁺ calcd 502.2, [M+H]⁺ found 502.3. ¹H NMR (400 MHz, CHLOROFORM-d) δ=9.47 (d, J=4 Hz, 1H), 8.44 (d, J=1.1 Hz, 1H), 8.23 (s, 1H), 7.98 (s, 1H), 7.88-7.80 (m, 1H), 7.72 (s, 1H), 7.36 (d, J=8.0 Hz, 1H), 7.07-6.93 (m, 1H), 6.09-5.69 (m, 1H), 5.24-4.93 (m, 1H), 4.68 (s, 2H), 4.09-3.97 (m, 1H), 3.85-3.73 (m, 2H), 2.77-2.67 (m, 1H), 2.40 (s, 3H), 1.94-1.76 (m, 1H), 1.69-1.53 (m, 1H)

Step 8: The mixture was separated by SFC (column: DAICEL CHIRALPAK AD (250 mm*30 mm, 10 μm); mobile phase: [CO₂-ACN/i-PrOH(0.1% NH₃H₂O)]; B %:75%, isocratic elution mode) to give I-500 and I-501. I-500 was further purified by prep-HPLC (column: Phenomenex luna C18 150*25 mm*10 um; mobile phase: [water(FA)-ACN]; gradient: 30%-60% B over 7 min) to give 7-(((R)-3,3-difluoro-2-hydroxypropoxy)methyl)-N-(5-(5-((1R,2S)-2-fluorocyclopropyl)-1,2,4-oxadiazol-3-yl)-2-methylphenyl)imidazo[1,2-a]pyridine-3-carboxamide (I-500) (17.47 mg, 98% purity) as white gum. MS (ESI): m/z for: C24H22F3N5O4 [M+H]⁺ calcd 502.2, [M+H]⁺ found. 502.3. ¹H NMR (400 MHz, METHANOL-d₄) δ=9.47 (d, J=4 Hz, 1H), 8.52 (s, 1H), 8.06 (d, J=4 Hz, 1H), 7.83-7.81 (m, 1H), 7.76 (s, 1H), 7.43 (d, J=8.0 Hz, 1H), 7.23-7.21 (m, 1H), 6.04-5.75 (m, 1H), 5.20-5.18 (m, 1H), 4.78-4.74 (m, 2H), 3.99-3.94 (m, 1H), 3.79-3.71 (m, 2H), 2.85-2.78 (m, 1H), 2.39 (s, 3H), 2.04-1.84 (m, 1H), 1.63-1.55 (m, 1H).

I-501 was further purified by prep-HPLC(column: Phenomenex luna C18 250*50 mm*15 um; mobile phase: [water(FA)-ACN]; gradient:30%-60% B over 7 min) to give 7-(((S)-3,3-difluoro-2-hydroxypropoxy)methyl)-N-(5-(5-((1R,2S)-2-fluorocyclopropyl)-1,2,4-oxadiazol-3-yl)-2-methylphenyl)imidazo[1,2-a]pyridine-3-carboxamide (I-501) (19.32 mg, 99.9% purity) as yellow solid. MS (ESI): m/z for: C24H22F3N5O4 [M+H]⁺ calcd 502.2, [M+H]⁺ found 502.3. ¹H NMR (400 MHz, METHANOL-d₄) δ=9.50 (d, J=8 Hz, 1H), 8.56 (s, 1H), 8.06 (d, J=4 Hz, 1H), 7.83-7.81 (m, 1H), 7.76 (s, 1H), 7.43 (d, J=8.0 Hz, 1H), 7.23-7.21 (m, 1H), 6.04-5.75 (m, 1H), 5.20-5.18 (m, 1H), 4.78-4.74 (m, 2H), 3.99-3.94 (m, 1H), 3.79-3.71 (m, 2H), 2.85-2.78 (m, 1H), 2.39 (s, 3H), 2.04-1.84 (m, 1H), 1.63-1.55 (m, 1H).

### Example 25—Preparation of N-(5-(5-((1R,2S)-2-fluorocyclopropyl)-1,2,4-oxadiazol-3-yl)-2-methylphenyl)-6-(2-(2-hydroxyethoxy)ethyl)pyrazolo[1,5-a]pyridine-3-carboxamide (I-503)

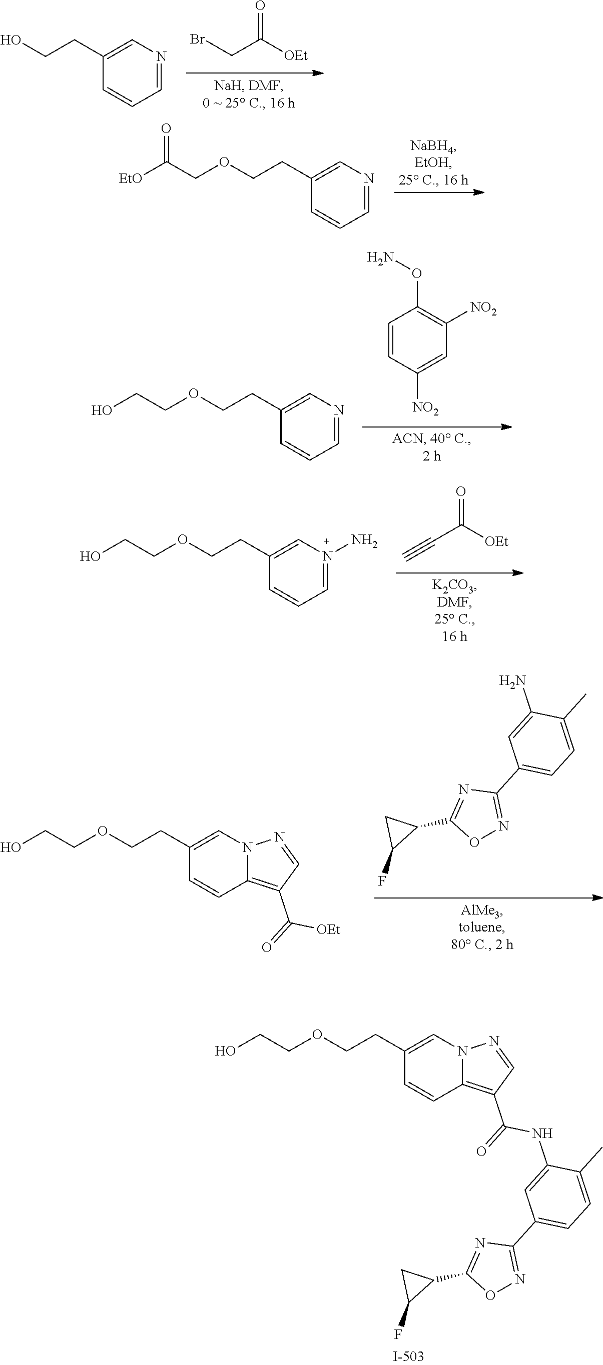

Step 1: To a solution of 2-(pyridin-3-yl)ethan-1-ol (5 g, 1.0 eq) in THE (80 mL) was added NaH (1.95 g, 60% purity, 1.2 eq) at 0° C., then the reaction mixture was stirred at 0° C. for 0.5 hours, then ethyl 2-bromoacetate (8.14 g, 1.2 eq) was added, the reaction mixture was stirred at 25° C. for 1.5 hours. The reaction mixture was quenched with H₂O 50 mL at 0° C., and extracted with ethyl acetate (100 mL×2). The combined organic layers were washed with brine (40 mL×2), dried over Na₂SO₄, filtered and concentrated under reduced pressure to give a residue. The residue was purified by column chromatography (SiO₂, Petroleum ether/ethyl acetate=5:1 to 0:1) to get compound ethyl 2-(2-(pyridin-3-yl)ethoxy)acetate (5.2 g) as a yellow oil. MS (ESI): m/z for C₁₁H₁₅NO₃, [M+H]⁺ calcd. 210.2, [MH]⁺ found 209.1. 1H NMR (400 MHz, CD₃OD) δ 8.46 (d, J=1.6 Hz, 1H), 8.37 (dd, J=1.2, 4.8 Hz, 1H), 7.82-7.76 (m, 1H), 7.36 (dd, J=4.8, 8.0 Hz, 1H), 4.23-4.13 (m, 2H), 4.09 (s, 2H), 3.78 (t, J=6.4 Hz, 2H), 2.95 (t, J=6.4 Hz, 2H), 1.25 (t, J=7.2 Hz, 3H).

Step 2: To a solution of ethyl 2-(2-(pyridin-3-yl)ethoxy)acetate (2 g, 1.0 eq) in EtOH (30 mL) was added NaBH₄ (1.08 g, 3.0 eq) at 0° C. The mixture was stirred at 25° C. for 12 hours. The mixture was quenched by addition water (100 mL) dropwise at 0° C. under N₂ atmosphere, then the mixture was stirred at 25° C. for 0.5 hour. The mixture was diluted with water (50 mL) and extracted with ethyl acetate (50 mL×3). The combined organic layers were washed with brine (30 mL×2), dried over Na₂SO₄, filtered and the filtrate was concentrated under reduced pressure to give a residue. The residue was purified by column chromatography (SiO₂, petroleum ether:ethyl acetate=5:1 to 0:1) to obtain 2-(2-(pyridin-3-yl)ethoxy)ethan-1-ol (1.2 g) as a colorless oil. MS (ESI): m/z for C₉H₁₃NO₂ [M+H]⁺ calcd. 167.8, [MH]⁺ found 167.1. ¹H NMR (400 MHz, CD₃OD) δ 8.44 (d, J=1.6 Hz, 1H), 8.37 (dd, J=1.2, 4.8 Hz, 1H), 7.82-7.75 (m, 1H), 7.36 (dd, J=4.8, 8.0 Hz, 1H), 3.72 (t, J=6.8 Hz, 2H), 3.66-3.62 (m, 2H), 3.55-3.50 (m, 2H), 2.93 (t, J=6.8 Hz, 2H).

Step 3:1-amino-3-(2-(2-hydroxyethoxy)ethyl)pyridin-1-ium: To a solution 2-(2-(pyridin-3-yl)ethoxy)ethan-1-ol (300 mg, 1.0 eq) in ACN (4 mL) was added 0-(2,4-dinitrophenyl)hydroxylamine (428 mg, 1.2 eq). The mixture was stirred at 40° C. for 12 hours. The reaction mixture was concentrated under reduced pressure to give 1-amino-3-(2-(2-hydroxyethoxy)ethyl)pyridin-1-ium (350 mg, crude) as a yellow oil which was used for next step with no further purification.

Step 4: To a solution of 1-amino-3-(2-(2-hydroxyethoxy)ethyl)pyridin-1-ium (320 mg, 1.0 eq) in DMF (3 mL) was added K₂CO₃ (48 mg, 2.0 eq) and ethyl propiolate (171 mg, 1.0 eq). The mixture was stirred at 25° C. for 12 hours. The reaction mixture was diluted with H₂O 50 mL and extracted with ethyl acetate (50 mL×3). The combined organic layers were washed with saturated brine (25 mL×2), dried over Na₂SO₄, filtered and concentrated under reduced pressure to give a residue. The residue was purified by column chromatography (SiO₂, petroleum ether:ethyl acetate=3:1 to 0:1) to give ethyl 6-(2-(2-hydroxyethoxy)ethyl)pyrazolo[1,5-a]pyridine-3-carboxylate (100 mg, 309.02 μmol, 17.69% yield) as a yellow oil. MS (ESI): m/z for C₁₄H₁₈N₂O₄. [M+H]⁺ calcd. 278.9, [MH]⁺ found 278.1. ¹H NMR (400 MHz, CDCl₃) δ 8.46-8.40 (m, 1H), 8.36 (s, 1H), 8.08 (d, J=9.2 Hz, 1H), 7.33 (dd, J=1.2, 8.8 Hz, 1H), 4.43-4.28 (m, 2H), 3.80-3.70 (m, 4H), 3.61-3.56 (m, 2H), 2.95 (t, J=6.4 Hz, 2H), 1.41 (t, J=7.2 Hz, 3H).

Step 5: To a solution of ethyl 6-(2-(2-hydroxyethoxy)ethyl)pyrazolo[1,5-a]pyridine-3-carboxylate (95 mg, 1.0 eq) and 5-(5-((1R,2S)-2-fluorocyclopropyl)-1,2,4-oxadiazol-3-yl)-2-methylaniline (79 mg, 1.0 eq) in toluene (2 mL) was added AlMe₃ (2 M, 2.5 eq) at 25° C. The mixture was stirred at 80° C. for 1 hour. The reaction mixture was quenched with NH₄Cl 30 mL then was extracted with ethyl acetate (40 mL×2). The combined organic layers were washed with brine (15 mL×2), dried over Na₂SO₄, filtered and concentrated under reduced pressure to give a residue. The residue was purified by prep-HPLC (neutral condition; column: Waters Xbridge 150*25 mm*5 um; mobile phase: [water(NH4HCO3)-ACN]; gradient: 30%-60% B over 9 min) to give N-(5-(5-((1R,2S)-2-fluorocyclopropyl)-1,2,4-oxadiazol-3-yl)-2-methylphenyl)-6-(2-(2-hydroxyethoxy)ethyl)pyrazolo[1,5-a]pyridine-3-carboxamide (33.11 mg) as an off-white solid. MS (ESI): m/z for C₂₄H₂₄FN₅O₄. [M+H]⁺ calcd. 278.9, [MH]⁺ found. 465.2. ¹H NMR (400 MHz, CD₃OD) δ=8.58 (d, J=4.0 Hz, 2H), 8.19 (d, J=9.2 Hz, 1H), 8.06 (d, J=1.6 Hz, 1H), 7.84 (dd, J=1.6, 8.0 Hz, 1H), 7.49 (dd, J=1.2, 9.2 Hz, 1H), 7.44 (d, J=8.0 Hz, 1H), 5.23-4.99 (m, 1H), 3.79 (t, J=6.3 Hz, 2H), 3.69-3.63 (m, 2H), 3.59-3.52 (m, 2H), 2.98 (t, J=6.0 Hz, 2H), 2.90-2.74 (m, 1H), 2.40 (s, 3H), 1.98-1.79 (m, 1H), 1.66-1.54 (m, 1H).

### Example 26—Preparation of N-[5-[5-[(1R,2S)-2-fluorocyclopropyl]-1,2,4-oxadiazol-3-yl]-2-methyl-phenyl]-7-[(E)-3-(2-hydroxyethoxy)allyl]imidazo[1,2-a]pyridine-3-carboxamide (I-506)

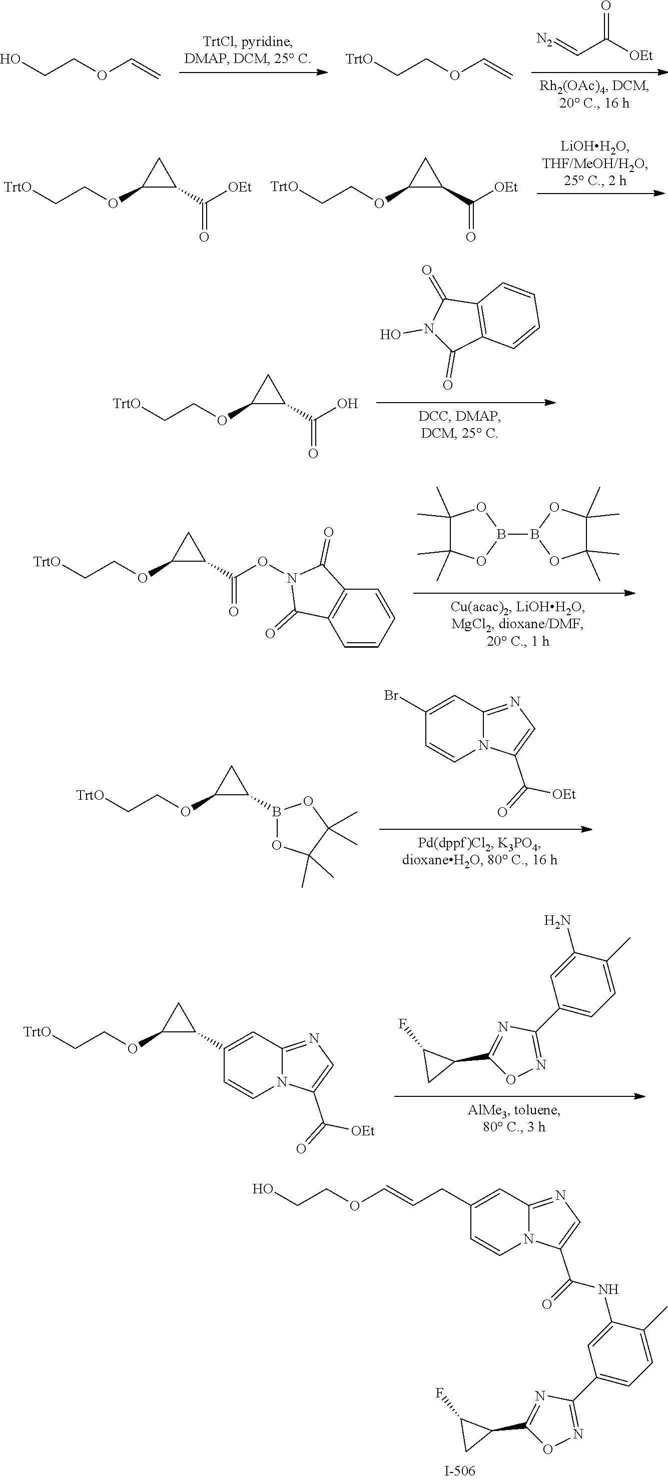

Step 1: To a solution of 2-(vinyloxy)ethan-1-ol (20 g, 227 mmol) and TrtCl (75.94 g, 272 mmol) in DCM (300 mL) was added DMAP (2.77 g, 22.7 mmol) and pyridine (35.9 g, 454 mmol, 36.6 mL). The mixture was stirred at 25° C. for 6 hrs. TLC (petroleum ether:ethyl acetate 10:1) indicated starting material was consumed completely and one new spot formed. The reaction was clean according to TLC. The mixture was concentrated in vacuo. The residue was purified by flash silica gel chromatography (petroleum ether:ethyl acetate 10:1). ((2-(vinyloxy)ethoxy)methanetriyl)tribenzene (24 g, 32% yield) was obtained as white solid. ¹H NMR 400 MHz, DMSO-d6 δ=7.25 (d, J=1.2 Hz, 15H), 6.67-6.48 (m, 1H), 4.29-4.17 (m, 1H), 4.05-3.96 (m, 1H), 3.91-3.80 (m, 2H), 3.22-3.10 (m, 2H).

Step 2: To a solution of ((2-(vinyloxy)ethoxy)methanetriyl)tribenzene (24 g, 72.6 mmol) and Rh(OAc)₂ (802 mg, 3.63 mmol) in the DCM (200 mL) was added ethyl 2-diazoacetate (16.6 g, 145 mmol) by syringe pump for 2 hrs (10 mL/h). The mixture was stirred at 0-20° C. for 10 hrs under N₂. TLC (petroleum ether:ethyl acetate 10:1) showed starting material was consumed completely. The mixture was concentrated in vacuo. The residue was purified by column chromatography (SiO₂, petroleum ether:ethyl acetate 100:0-10:1, TLC: petroleum ether:ethyl acetate 10:1). ((2-(vinyloxy)ethoxy)methanetriyl)tribenzene (10 g, 33% yield) was obtained as colorless oil.

Step 3: To a solution of ((2-(vinyloxy)ethoxy)methanetriyl)tribenzene (10 g, 24.0 mmol) in THE (50 mL) and MeOH (50 mL) and H₂O (50 mL) was added LiOH·H₂O (3.0 g, 72.0 mmol). The mixture was stirred at 25° C. for 2 hr. TLC (petroleum ether:ethyl acetate 10:1) showed starting material was consumed completely. The reaction mixture was concentrated under reduced pressure to remove most solvent. The residue was diluted with 1M HCl to pH=3-4 and extracted with ethyl acetate (30 mL×3). The combined organic layers were washed with brine 50 mL, dried over Na₂SO₄, filtered and concentrated under reduced pressure to give a residue. (1S,2S)-2-(2-(trityloxy)ethoxy)cyclopropane-1-carboxylic acid (10 g, 96% yield, 90% purity) was obtained as white solid without further purification. ¹H NMR 400 MHz, CDCl3-d δ =7.52-7.44 (m, 6H), 7.34-7.24 (m, 9H), 4.18-4.11 (m, 2H), 3.78-3.69 (m, 3H), 3.29-3.21 (m, 2H), 1.87-1.79 (m, 1H), 1.33-1.30 (m, 2H), 1.29-1.26 (m, 3H).

Step 4: To a solution of (1S,2S)-2-(2-(trityloxy)ethoxy)cyclopropane-1-carboxylic acid(10.0 g, 25.7 mmol) and 2-hydroxyisoindoline-1,3-dione(6.30 g, 38.6 mmol) in DCM (150 mL) was added DCC (10.6 g, 51.4 mmol, 10.4 mL). The mixture was stirred at 20° C. for 6 hr. TLC (petroleum ether:ethyl acetate 10:1) indicated Reactant 1 was consumed completely and one new spot formed. The reaction was clean according to TLC. The mixture was concentrated in vacuo. The residue was purified by column chromatography (SiO2, petroleum ether:ethyl acetate 10:1). 1,3-dioxoisoindolin-2-yl (1S,2S)-2-(2-(trityloxy)ethoxy)cyclopropane-1-carboxylate (10.5 g, 76% yield) was obtained as white solid. 1H NMR 400 MHz, CDCl3-d δ=7.95-7.88 (m, 2H), 7.85-7.78 (m, 2H), 3.98-3.89 (m, 1H), 3.88-3.72 (m, 2H), 2.23-2.06 (m, 1H), 1.69-1.60 (m, 1H).

Step 5: To a 15 mL culture tube equipped with a stir bar were added (1,3-dioxoisoindolin-2-yl) (1S,2S)-2-(2-trityloxyethoxy)cyclopropanecarboxylate (5 g, 0.14 M, 73.63 mL), 4,4,5,5-tetramethyl-2-(4,4,5,5-tetramethyl-1,3,2-dioxaborolan-2-yl)-1,3,2-dioxaborolane (7.85 g, 30.9 mmol), LiOH·H₂O (6.49 g, 154 mmol), bis[(Z)-1-methyl-3-oxo-but-1-enoxy]copper (809 mg, 3.09 mmol) and MgCl₂ (1.47 g, 15.4 mmol). The tube was evacuated and backfilled with N₂ for 3 times. MTBE (35 mL)/DMF (35 mL) (6:1-1:2 ratio, 0.14 M) was added and the resulting mixture was stirred under 1000 rpm at 25° C. for 30 min until dark brown color. TLC (petroleum ether:ethyl acetate 10:1) indicated many new spots with lower polarity was detected. The residue was diluted with sat.NH₄Cl.aq (3 mL) and extracted with ethyl acetate (10 mL×3). The combined organic layers were washed with brine 20 mL, dried over Na₂SO₄, filtered and concentrated under reduced pressure to give a residue. The residue was purified by column chromatography (SiO2, Petroleum ether/ethyl acetate=100/1 to 10/1). 4,4,5,5-tetramethyl-2-((1S,2S)-2-(2-(trityloxy)ethoxy)cyclopropyl)-1,3,2-dioxaborolane was obtained as yellow solid. ¹H NMR 400 MHz, CDCl₃-d δ=7.48-7.45 (m, 6H), 7.33-7.27 (m, 7H), 7.25-7.20 (m, 3H), 3.69 (t, J=5.2 Hz, 2H), 3.55-3.45 (m, 1H), 3.20 (t, J=5.2 Hz, 2H), 1.22 (s, 11H), 1.00-0.92 (m, 1H), 0.76-0.67 (m, 1H), 0.22-0.12 (m, 1H).

Step 6: A mixture of 4,4,5,5-tetramethyl-2-[(1S,2S)-2-(2-trityloxyethoxy)cyclopropyl]-1,3,2-dioxaborolane (430 mg, 914.10 μmol), ethyl 7-bromoimidazo[1,2-a]pyridine-3-carboxylate (245.98 mg, 914.10 μmol), XPhos Pd G₃ (77.37 mg, 91.41 μmol), Na₂CO₃ (193.77 mg, 1.83 mmol) in dioxane (5 mL) and H₂O (1 mL) was degassed and purged with N₂ for 3 times, and then the mixture was stirred at 80° C. for 16 hr under N₂ atmosphere. LC-MS showed 30% desired mass was detected, diluted with water 10 mL and extracted with ethyl acetate 30 mL (10 mL*3). The combined organic layers were washed with brine (10 ml), dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; gradient: 70%-100% B over 12 min). ethyl 7-((1R,2S)-2-(2-(trityloxy)ethoxy)cyclopropyl)imidazo[1,2-a]pyridine-3-carboxylate (160 mg, 240.32 μmol, 26.29% yield, 80% purity) was obtained as yellow solid. LCMS product: RT=0.617 min, m/z=533.3 (M+H)+.

Step 7: To a solution of ethyl 7-[(1R,2S)-2-(2-trityloxyethoxy)cyclopropyl]imidazo[1,2-a]pyridine-3-carboxylate (100 mg, 187 μmol) and 5-[5-[(1R,2S)-2-fluorocyclopropyl]-1,2,4-oxadiazol-3-yl]-2-methyl-aniline (43.79 mg, 187.75 μmol) in ToI. (3 mL) was added AlMe₃ (2 M, 234.69 μL). The mixture was stirred at 80° C. for 3 hr. LC-MS showed starting material was consumed completely and desired mass was detected. The reaction mixture was quenched by addition sat.NH₄Cl.aq mL at 0° C., and extracted with ethyl acetate (20 mL×3). The combined organic layers were washed with brine 20 mL, dried over Na₂SO₄, filtered and concentrated under reduced pressure to give a residue. The residue was purified by prep-HPLC (column: Welch Xtimate C18 150*25 mm*5 um; mobile phase: [water(FA)-ACN]; gradient: 25%-45% B over 10 min) to give N-[5-[5-[(1R,2S)-2-fluorocyclopropyl]-1,2,4-oxadiazol-3-yl]-2-methyl-phenyl]-7-[(E)-3-(2-hydroxyethoxy)allyl]imidazo[1,2-a]pyridine-3-carboxamide (0.01 g, 10.60% yield, 95% purity) as white solid. LCMS product: RT=0.480 min, m/z=478.2 (M+H)+. ¹H NMR: 400 MHz, DMSO-d δ=9.97 (s, 1H), 9.35 (d, J=7.1 Hz, 1H), 8.54 (s, 1H), 8.03 (d, J=1.6 Hz, 1H), 7.82-7.73 (m, 1H), 7.55 (s, 1H), 7.48 (d, J=8.0 Hz, 1H), 7.08-7.01 (m, 1H), 6.56 (d, J=12.4 Hz, 1H), 5.41-5.18 (m, 1H), 5.02-4.88 (m, 1H), 4.77 (br s, 1H), 3.76-3.68 (m, 2H), 3.59 (br s, 2H), 3.36 (s, 2H), 3.13-2.99 (m, 1H), 2.36 (s, 3H), 2.02-1.87 (m, 1H), 1.65-1.52 (m, 1H)

### Example 27—Preparation of 7-methyl-N-[2-methyl-5-[5-[rel-(3S)-4,4,4-trifluoro-3-hydroxy-3-methyl-butyl]-1,2,4-oxadiazol-3-yl]phenyl]imidazo[1,2-a]pyridine-3-carboxamide (I-516) and 7-methyl-N-[2-methyl-5-[5-[rel-(3R)-4,4,4-trifluoro-3-hydroxy-3-methyl-butyl]-1,2,4-oxadiazol-3-yl]phenyl]imidazo[1,2-a]pyridine-3-carboxamide (I-515)

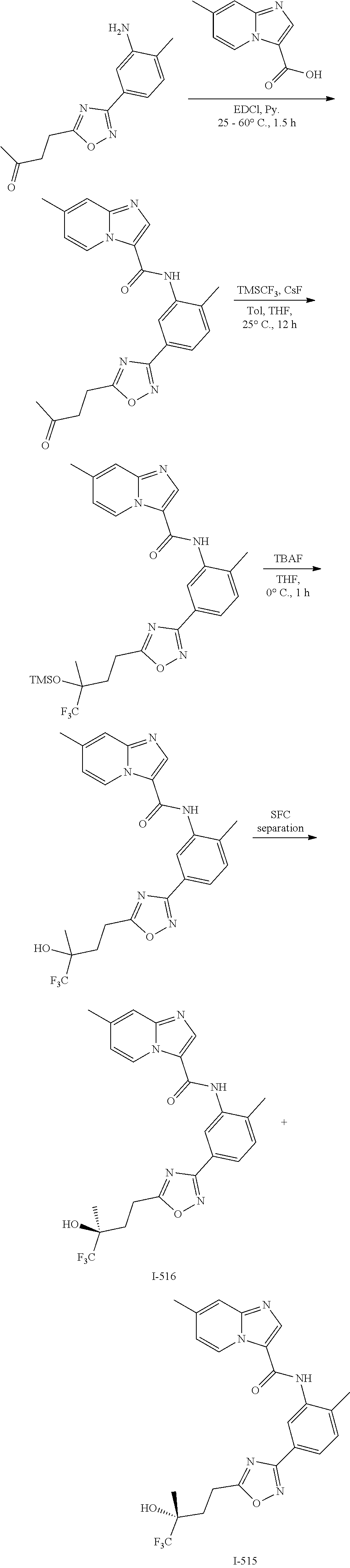

Step 1: To a solution of 7-methylimidazo[1,2-a]pyridine-3-carboxylic acid (500 mg, 1.0 eq) in pyridine (5 mL) was added EDCI (870 mg, 1.6 eq), the reaction mixture was stirred at 25° C. for 0.5 hour, then 4-(3-(3-amino-4-methylphenyl)-1,2,4-oxadiazol-5-yl)butan-2-one (765 mg, 1.1 eq) was added, the reaction mixture was stirred at 60° C. for 1 hour. The reaction mixture was diluted with H₂O 50 mL and extracted with ethyl acetate (50 mL×2). The combined organic layers were washed with brine (30 mL×3), dried over Na₂SO₄, filtered and concentrated under reduced pressure to give a residue. The residue was purified by column chromatography (SiO₂, petroleum ether/ethyl acetate=5/1 to 1/1) to give 7-methyl-N-(2-methyl-5-(5-(3-oxobutyl)-1,2,4-oxadiazol-3-yl)phenyl)imidazo[1,2-a]pyridine-3-carboxamide (540 mg) as a white solid. ¹H NMR (400 MHz, METHANOL-d₄) δ=9.36 (d, J=7.2 Hz, 1H), 8.40 (s, 1H), 8.07 (d, J=1.6 Hz, 1H), 7.91-7.82 (m, 1H), 7.50 (d, J=0.8 Hz, 1H), 7.45 (d, J=8.0 Hz, 1H), 7.05-7.00 (m, 1H), 3.20-3.10 (m, 4H), 2.49 (s, 3H), 2.40 (s, 3H), 2.22 (s, 3H).

Step 2: To a solution of 7-methyl-N-(2-methyl-5-(5-(3-oxobutyl)-1,2,4-oxadiazol-3-yl)phenyl)imidazo[1,2-a]pyridine-3-carboxamide (200 mg, 1.0 eq) and CsF (19 mg, 0.25 eq) in toluene (15 mL) and THF (2 mL) was added TMSCF3 (705 mg, 10 eq) dropwise. The mixture was stirred at 25° C. for 12 hours under N₂. The reaction mixture was used for the next step directly. 7-methyl-N-(2-methyl-5-(5-(4,4,4-trifluoro-3-methyl-3-((trimethylsilyl)oxy)butyl)-1,2,4-oxadiazol-3-yl)phenyl)imidazo[1,2-a]pyridine-3-carboxamide (270 mg, crude) was obtained as a yellow liquid. MS (ESI): m/z for C₂₆H₃₀F₃N₅O₃Si [M+H]+ calcd. 546.6 [M+H]+ found 546.2.

Step 3: To a solution of 7-methyl-N-(2-methyl-5-(5-(4,4,4-trifluoro-3-methyl-3-((trimethylsilyl)oxy)butyl)-1,2,4-oxadiazol-3-yl)phenyl)imidazo[1,2-a]pyridine-3-carboxamide (150 mg, 1.0 eq) in THE (4 mL) was added TBAF (1 M, 1.5 eq) at 0° C. The mixture was stirred at 0° C. for 1 hour. The reaction mixture was diluted with H₂O (30 mL) and extracted with ethyl acetate (40 mL×3). The combined organic layers were washed with brine (25 mL×2), dried over Na₂SO₄, filtered and concentrated under reduced pressure to give a residue. The residue was purified by column chromatography (SiO₂, Petroleum ether/ethyl acetate=10/1 to 1/1) to give 7-methyl-N-(2-methyl-5-(5-(4,4,4-trifluoro-3-hydroxy-3-methylbutyl)-1,2,4-oxadiazol-3-yl)phenyl)imidazo[1,2-a]pyridine-3-carboxamide (79.03 mg) as a yellow solid. ¹H NMR (400 MHz, METHANOL-d₄) δ=9.37 (d, J=7.2 Hz, 1H), 8.41 (s, 1H), 8.10 (d, J=1.6 Hz, 1H), 7.92-7.85 (m, 1H), 7.50 (s, 1H), 7.46 (d, J=8.0 Hz, 1H), 7.05-7.00 (m, 1H), 3.17-3.11 (m, 2H), 2.49 (s, 3H), 2.40 (s, 3H), 2.29 (s, 1H), 2.18 (s, 1H), 1.39 (s, 3H). MS (ESI): m/z for C₂₃H₂₂F₃N₅O₃[M+H]+ calcd. 474.2 [M+H]+ found 474.4.

Step 4: The 7-methyl-N-(2-methyl-5-(5-(4,4,4-trifluoro-3-hydroxy-3-methylbutyl)-1,2,4-oxadiazol-3-yl)phenyl)imidazo[1,2-a]pyridine-3-carboxamide (60 mg, 1.0 eq) was purified by SFC separation (column: DAICEL CHIRALCEL OX (250 mm*30 mm, 10 um); mobile phase: [CO₂-EtOH (0.1% NH₃H₂O)]; B %: 40%, isocratic elution mode) to give two peaks. These two peaks were further purified by prep-HPLC (FA condition; column: C18 150×30 mm; mobile phase: [water(FA)-ACN]; gradient: 25%-55% B over 7 min) to give 7-methyl-N-[2-methyl-5-[5-[rel-(3 S)-4,4,4-trifluoro-3-hydroxy-3-methyl-butyl]-1,2,4-oxadiazol-3-yl]phenyl]imidazo[1,2-a]pyridine-3-carboxamide (I-516) (10.85 mg) as a white solid and 7-methyl-N-[2-methyl-5-[5-[rel-(3R)-4,4,4-trifluoro-3-hydroxy-3-methyl-butyl]-1,2,4-oxadiazol-3-yl]phenyl]imidazo[1,2-a]pyridine-3-carboxamide (I-515) (15.39 mg) as a white solid. MS (ESI): m/z for C₂₃H₂₂F₃N₅O₃, [M+H]⁺ calcd. 474.1, [M+H]⁺ found 474.1. 7-methyl-N-[2-methyl-5-[5-[rel-(3 S)-4,4,4-trifluoro-3-hydroxy-3-methyl-butyl]-1,2,4-oxadiazol-3-yl]phenyl]imidazo[1,2-a]pyridine-3-carboxamide: ¹H NMR (400 MHz, METHANOL-d₄) δ=9.38 (d, J=7.2 Hz, 1H), 8.41 (s, 1H), 8.11 (d, J=1.6 Hz, 1H), 7.90 (dd, J=1.6, 8.0 Hz, 1H), 7.51 (s, 1H), 7.47 (d, J=8.0 Hz, 1H), 7.04 (dd, J=1.2, 7.2 Hz, 1H), 3.15 (t, J=8.0 Hz, 2H), 2.50 (s, 3H), 2.41 (s, 3H), 2.35-2.25 (m, 1H), 2.22-2.12 (m, 1H), 1.39 (s, 3H). 7-methyl-N-[2-methyl-5-[5-[rel-(3R)-4,4,4-trifluoro-3-hydroxy-3-methyl-butyl]-1,2,4-oxadiazol-3-yl]phenyl]imidazo[1,2-a]pyridine-3-carboxamide: ¹H NMR (400 MHz, METHANOL-d₄) δ=9.37 (d, J=7.2 Hz, 1H), 8.41 (s, 1H), 8.10 (d, J=1.6 Hz, 1H), 7.89 (dd, J=1.6, 8.0 Hz, 1H), 7.51 (s, 1H), 7.46 (d, J=8.0 Hz, 1H), 7.06-7.01 (m, 1H), 3.15 (t, J=8.0 Hz, 2H), 2.49 (s, 3H), 2.40 (s, 3H), 2.36-2.24 (m, 1H), 2.18 (t, J=8.0 Hz, 1H), 1.39 (s, 3H).

### Example 28—Preparation of N-(5-(5-(2-hydroxy-2-phenylethyl)-1,2,4-oxadiazol-3-yl)-2-methylphenyl)imidazo[1,2-a]pyridine-3-carboxamide (I-517)

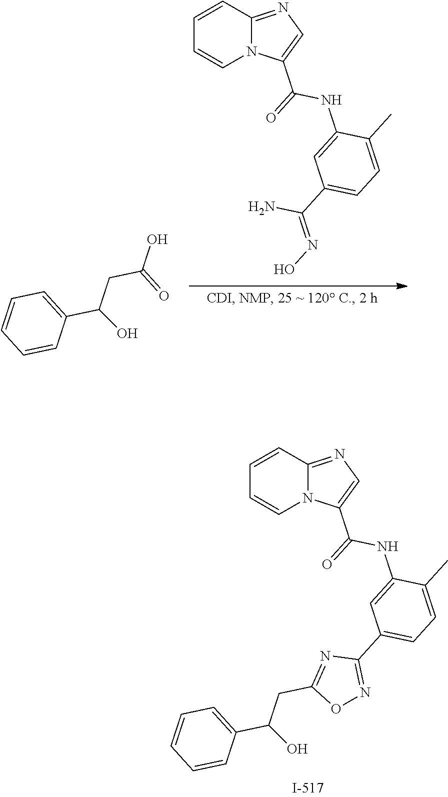 [SMILES: O=C(O)CC(O)c1ccccc1 ; Cc1ccc(/C(N)=N/O)cc1NC(=O)c1cnc2ccccn12 ; Cc1ccc(-c2noc(CC(O)c3ccccc3)n2)cc1NC(=O)c1cnc2ccccn12]

Step 1: To a solution of 3-hydroxy-3-phenyl-propanoic acid (150 mg, 1.0 eq) in NMP (2 mL) was added CDI (153 mg, 1.05 eq). The mixture was stirred at 25° C. for 15 minutes. To the mixture was added N-[5-[(Z)—N′-hydroxycarbamimidoyl]-2-methyl-phenyl]imidazo[1,2-a]pyridine-3-carboxamide (279 mg, 1.0 eq) and stirred at 25° C. for 5 minutes. The mixture was stirred at 120° C. for 2 hours. The mixture was filtered to give a clean solution. The mixture was purified by prep-HPLC (column: C18 150×30 mm; mobile phase: [water(FA)-ACN]; gradient: 28%-58% B over 7 min) to give N-(5-(5-(2-hydroxy-2-phenylethyl)-1,2,4-oxadiazol-3-yl)-2-methylphenyl)imidazo[1,2-a]pyridine-3-carboxamide (I-517) (140.83 mg, 35% yield) as yellow solid. ¹H NMR (400 MHz, DMSO-d₆) δ=10.03 (s, 1H), 9.47 (d, J=4.0 Hz, 1H), 8.61 (s, 1H), 8.10 (d, J=2.0 Hz, 1H), 7.83-7.78 (m, 2H), 7.57-7.48 (m, 2H), 7.47-7.41 (m, 2H), 7.39-7.33 (m, 2H), 7.31-7.25 (m, 1H), 7.22-7.14 (m, 1H), 5.80 (d, J=4.0 Hz, 1H), 5.18-5.13 (m, 1H), 3.34-3.32 (m, 1H), 2.36 (s, 3H). MS (ESI): m/z for C₂₅H₂₁N₅O₃ [M+H]⁺ calcd. 440.16, [M+H]⁺ found 440.3.

### Example 29—Preparation of methyl N-(5-(5-(3,3-difluorocyclobutyl)-1,2,4-oxadiazol-3-yl)-2-methylphenyl)pyrazolo[1,5-a]pyridine-3-carboxamide (I-518)

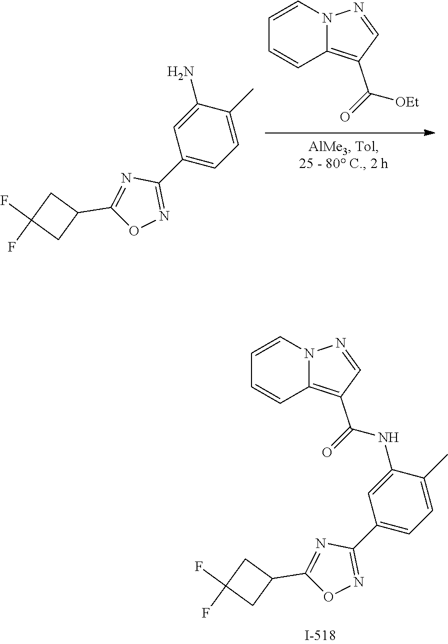

Step 1: A mixture of 5-(5-(3,3-difluorocyclobutyl)-1,2,4-oxadiazol-3-yl)-2-methylaniline (100 mg, 1 eq) and trimethylaluminum (2 M, 2.5 eq) in toluene (2 mL) was stirred at 25° C. for 1 hour under N₂, then 5-(5-(3,3-difluorocyclobutyl)-1,2,4-oxadiazol-3-yl)-2-methylaniline (72 mg, 1.0 eq) was added into, the resultant mixture was stirred at 80° C. for 1 hour. The reaction mixture was quenched by addition saturated ammonium chloride in water 20 mL at 0° C., and then diluted with H₂O (20 mL) and extracted with DCM (40 mL×3). The combined organic layers were washed with brine (25 mL×2), dried over Na₂SO₄ filtered and concentrated under reduced pressure to give a residue. The residue was purified by prep-HPLC (column: C18 150×30 mm; mobile phase: [water(FA)-ACN]; gradient:48%-78% B over 7 min) to give: methyl N-(5-(5-(3,3-difluorocyclobutyl)-1,2,4-oxadiazol-3-yl)-2-methylphenyl)pyrazolo[1,5-a]pyridine-3-carboxamide (78.15 mg) as a white solid. ¹H NMR (400 MHz, DMSO-d6) δ=9.76 (s, 1H), 8.86 (d, J=7.2 Hz, 1H), 8.78 (s, 1H), 8.25 (d, J=8.8 Hz, 1H), 8.11 (d, J=1.4 Hz, 1H), 7.81 (dd, J=1.6, 8.0 Hz, 1H), 7.59-7.45 (m, 2H), 7.18-7.08 (m, 1H), 3.93-3.84 (m, 1H), 3.24-3.05 (m, 4H), 2.37 (s, 3H). MS (ESI): m/z for C₂₁H₁₇F₂N₅O₂ [M+H]⁺ calcd. 410.4 [M+H]⁺ found 410.1.

### Example 30—Preparation of N-(5-(5-(2-hydroxy-3-methylbutyl)-1,2,4-oxadiazol-3-yl)-2-methylphenyl)imidazo[1,2-a]pyridine-3-carboxamide (I-519)

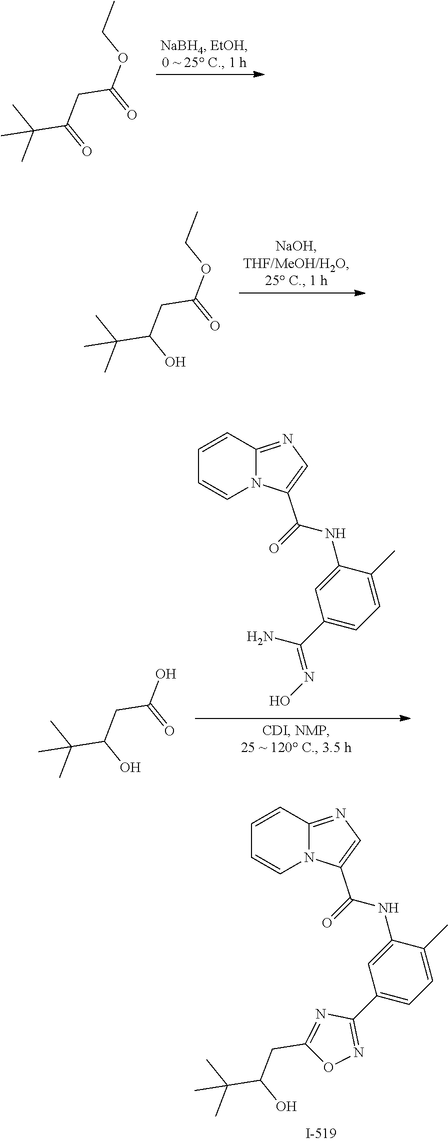

Step 1: To a solution of ethyl 4,4-dimethyl-3-oxo-pentanoate (2.0 g, 1.0 eq) in EtOH (20 mL) was added NaBH₄ (220 mg, 0.5 eq) at 0° C. under N₂ atmosphere. The mixture was stirred at 25° C. for 1 hour under N₂ atmosphere. The reaction mixture was quenched by water 10 mL at 0° C., and then diluted with water (40 mL) and extracted with ethyl acetate (80 mL×3). The combined organic layers were washed with brine (50 mL×3), dried over Na₂SO₄, filtered and concentrated under reduced pressure to give a residue. The residue was purified by column chromatography (SiO₂, petroleum ether/ethyl acetate=5/1) to ethyl 3-hydroxy-4,4-dimethylpentanoate (760 mg, 38% yield) as a colorless oil. ¹H NMR (400 MHz, DMSO-d₆) δ=4.73 (d, J=6.0 Hz, 1H), 4.09-4.01 (m, 2H), 3.57-3.51 (m, 1H), 2.47-2.40 (m, 1H), 2.18-2.10 (m, 1H), 1.18 (t, J=7.2 Hz, 3H), 0.81 (s, 9H).

Step 2: To a solution of ethyl 3-hydroxy-4,4-dimethyl-pentanoate (360 mg, 1.0 eq) in THF (1 mL), MeOH (1 mL) and H₂O (1 mL) was added NaOH (165 mg, 2.0 eq). The mixture was stirred at 25° C. for 1 hour. The reaction mixture was poured into water (10 mL), and then the pH of the solution was adjusted to 4˜ 5 with 1N HCl, then the mixture was extracted with ethyl acetate (30 mL×3), dried over Na₂SO₄, filtered and concentrated under reduced pressure to give 3-hydroxy-4,4-dimethylpentanoic acid (250 mg, 83% yield) as a white solid. ¹H NMR (400 MHz, DMSO-d₆) δ=3.56-3.49 (m, 1H), 2.41-2.32 (m, 1H), 2.11-2.02 (m, 1H), 0.81 (s, 9H).

Step 3: To a solution of 3-hydroxy-4,4-dimethyl-pentanoic acid (250 mg, 1.0 eq) in NMP (3 mL) was added di(imidazol-1-yl)methanone (333 mg, 1.2 eq). The mixture was stirred at 25° C. for 0.5 hour. The mixture was used into the next step without further work up. Compound 3-hydroxy-1-imidazol-1-yl-4,4-dimethyl-pentan-1-one (335 mg, 100% yield) in NMP 3 mL was obtained as a black liquid and used for the next step directly.

To a solution of 3-hydroxy-1-imidazol-1-yl-4,4-dimethyl-pentan-1-one (335 mg, 1.0 eq) in NMP (3 mL) was added N-[5-[(Z)—N′-hydroxycarbamimidoyl]-2-methyl-phenyl]imidazo[1,2-a]pyridine-3-carboxamide (528 mg, 1.0 eq). The mixture was stirred at 120° C. for 3 hours. The reaction was purified directly without work-up. The residue was purified by prep-HPLC (FA condition; column: Phenomenex luna C18 150*40 mm*15 um; mobile phase: [water(FA)-ACN]; gradient: 22%-52% B over 15 min) to give N-(5-(5-(2-hydroxy-3-methylbutyl)-1,2,4-oxadiazol-3-yl)-2-methylphenyl)imidazo[1,2-a]pyridine-3-carboxamide (I-519) (377.37 mg, 47% yield) as a white solid. ¹H NMR (400 MHz, DMSO-d₆) δ=10.02 (s, 1H), 9.46 (d, J=7.2 Hz, 1H), 8.59 (s, 1H), 8.09 (d, J=1.6 Hz, 1H), 7.85-7.76 (m, 2H), 7.55-7.46 (m, 2H), 7.20-7.15 (m, 1H), 5.03 (d, J=6.4 Hz, 1H), 3.77-3.67 (m, 1H), 3.17-3.10 (m, 1H), 2.93-2.84 (m, 1H), 2.37 (s, 3H), 0.91 (s, 9H). MS (ESI): m/z for C₂₃H₂₅N₅O₃ [M+H]⁺ calcd. 420.20, [MH]⁺ found 420.2.

### Example 31—Preparation of N-(5-(5-(3,3-difluorocyclobutyl)-1,2,4-oxadiazol-3-yl)-2-methylphenyl)-7-((2-hydroxy-2-methylpropoxy)methyl)imidazo[1,2-a]pyridine-3-carboxamide (I-521)

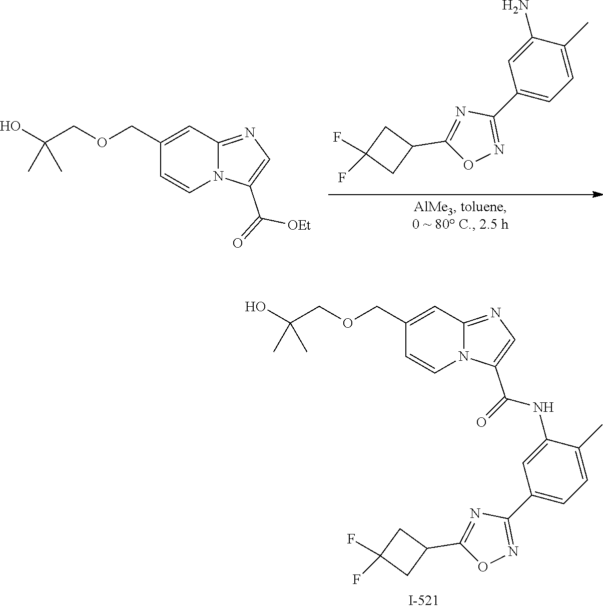 [SMILES: CCOC(=O)c1cnc2cc(COCC(C)(C)O)ccn12 ; Cc1ccc(-c2noc(C3CC(F)(F)C3)n2)cc1N ; Cc1ccc(-c2noc(C3CC(F)(F)C3)n2)cc1NC(=O)c1cnc2cc(COCC(C)(C)O)ccn12]

Step 1: To a solution of 5-[5-(3,3-difluorocyclobutyl)-1,2,4-oxadiazol-3-yl]-2-methyl-aniline (122 mg, 0.9 eq) in the toluene (2 mL) was added AlMe₃ (2 M, 2.5 eq) at 0° C. The mixture was stirred at 25° C. for 0.5 hour under N₂ atmosphere. Then ethyl 7-[(2-hydroxy-2-methyl-propoxy)methyl]imidazo[1,2-a]pyridine-3-carboxylate (150 mg, 1.0 eq) was added, the mixture was stirred at 80° C. for 2 hours under N₂ atmosphere. The reaction mixture was quenched by addition NH₄Cl solution (1 mL) at 0° C. and filtered, the filtrate was diluted with water 10 mL and extracted with ethyl acetate (20 mL×3). The combined organic layers were washed with brine (10 mL×3), dried over Na₂SO₄, filtered and concentrated under reduced pressure to give a residue. The residue was purified by prep-HPLC (basic condition; column: Waters Xbridge 150*25 mm*5 μm; mobile phase: [water(NH3H2O)-ACN]; gradient: 34%-64% B over 9 min) to give N-(5-(5-(3,3-difluorocyclobutyl)-1,2,4-oxadiazol-3-yl)-2-methylphenyl)-7-((2-hydroxy-2-methylpropoxy)methyl)imidazo[1,2-a]pyridine-3-carboxamide (I-521) (79.15 mg, 29% yield) was obtained as a white solid. ¹H NMR (400 MHz, DMSO-d₆) δ=10.01 (s, 1H), 9.40 (d, J=7.2 Hz, 1H), 8.56 (s, 1H), 8.08 (d, J=1.2 Hz, 1H), 7.85-7.80 (m, 1H), 7.72 (s, 1H), 7.50 (d, J=8.0 Hz, 1H), 7.16-7.09 (m, 1H), 4.65 (s, 2H), 4.45 (s, 1H), 3.94-3.82 (m, 1H), 3.27 (s, 2H), 3.23-3.01 (m, 4H), 2.36 (s, 3H), 1.14 (s, 6H). MS (ESI): m/z for C26H27F2N5O4 [M+H]⁺ calcd. 512.20, [M+H]⁺ found 512.2.

### Example 32—Preparation of N-(5-(5-(2-hydroxy-3-methylbutyl)-1,2,4-oxadiazol-3-yl)-2-methylphenyl)imidazo[1,2-a]pyridine-3-carboxamide (I-525)

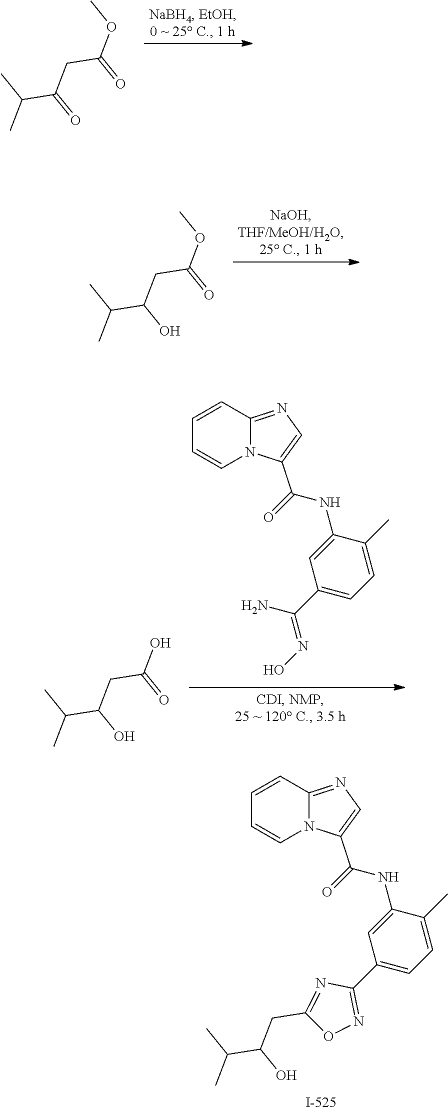

Step 1: To a solution of methyl 4-methyl-3-oxo-pentanoate (2.0 g, 1.0 eq) in MeOH (20 mL) was added NaBH₄ (262 mg, 0.5 eq) at 0° C. under N₂ atmosphere. The mixture was stirred at 25° C. for 1 hour under N₂ atmosphere. The reaction mixture was quenched by NH₄Cl 10 mL at 0° C., and then diluted with water 40 mL and extracted with ethyl acetate (80 mL×3). The combined organic layers were washed with brine (50 mL×3), dried over Na₂SO₄, filtered and concentrated under reduced pressure to give a residue. The residue was purified by column chromatography (SiO₂, Petroleum ether/ethyl acetate=5/1) to methyl 3-hydroxy-4-methylpentanoate (2 g, 99% yield) as a colorless oil. ¹H NMR (400 MHz, DMSO-d₆) δ=4.66 (d, J=6.0 Hz, 1H), 3.70-3.60 (m, 1H), 3.58 (s, 3H), 2.45-2.38 (m, 1H), 2.27-2.19 (m, 1H), 1.63-1.50 (m, 1H), 0.86-0.80 (m, 6H).

Step 2: To a solution of methyl 3-hydroxy-4-methyl-pentanoate (300 mg, 1.0 eq) in THF (1 mL), MeOH (1 mL) and H₂O (1 mL) was added NaOH (164 mg, 2.0 eq). The mixture was stirred at 25° C. for 1 hour. The reaction mixture was poured into water (10 ml), and then the pH of the solution was adjusted to 4˜5 with 1N HCl (6 ml), then the mixture was extracted with ethyl acetate (30 mL×3), dried over Na₂SO₄, filtered and concentrated under reduced pressure to give 3-hydroxy-4-methylpentanoic acid (250 mg, 92% yield) as a colorless oil. ¹H NMR (400 MHz, DMSO-d₆) δ=12.19-11.70 (m, 1H), 4.57 (s, 1H), 3.68-3.57 (m, 1H), 2.36-2.28 (m, 1H), 2.20-2.10 (m, 1H), 1.64-1.50 (m, 1H), 0.86-0.80 (m, 6H).

Step 3: To a solution of 3-hydroxy-4-methyl-pentanoic acid (150 mg, 1.0 eq) in NMP (2 mL) was added di(imidazol-1-yl)methanone (221 mg, 1.2 eq). The mixture was stirred at 25° C. for 0.5 hour. The mixture was used into the next step without further work up. Compound 3-hydroxy-1-imidazol-1-yl-4-methyl-pentan-1-one (206 mg, 100% yield) in NMP (2 mL) was obtained as a black liquid and used for the next step directly. To a solution of 3-hydroxy-1-imidazol-1-yl-4-methyl-pentan-1-one (206 mg, 1.0 eq) in NMP (2 mL) was added N-[5-[(Z)—N′-hydroxycarbamimidoyl]-2-methyl-phenyl]imidazo[1,2-a]pyridine-3-carboxamide (350 mg, 1.0 eq). The mixture was stirred at 120° C. for 3 hours. The reaction was purified directly without work-up. The residue was purified by prep-HPLC (FA condition; column: YMC-Actus Triart C18 150*30 mm*7 um; mobile phase: [water(FA)-ACN]; gradient: 28%-68% B over 10 min) to give N-(5-(5-(2-hydroxy-3-methylbutyl)-1,2,4-oxadiazol-3-yl)-2-methylphenyl)imidazo[1,2-a]pyridine-3-carboxamide (I-525) (212.85 mg, 40% yield) as a white solid. ¹H NMR (400 MHz, DMSO-d₆) δ=10.01 (s, 1H), 9.46 (d, J=7.2 Hz, 1H), 8.59 (s, 1H), 8.08 (s, 1H), 7.84-7.76 (m, 2H), 7.56-7.46 (m, 2H), 7.18 (t, J=7.2 Hz, 1H), 4.96 (d, J=5.6 Hz, 1H), 3.83-3.75 (m, 1H), 3.15-3.07 (m, 1H), 3.01-2.92 (m, 1H), 2.37 (s, 3H), 1.75-1.64 (m, 1H), 0.92 (t, J=6.4 Hz, 6H). MS (ESI): m/z for C₂₂H₂₃N₅O₃ [M+H]⁺ calcd. 406.2, [M+H]⁺ found 406.2.

### Example 33—Preparation of N-(5-(5-(3-(difluoromethyl)-3-hydroxycyclobutyl)-1,2,4-oxadiazol-3-yl)-2-methylphenyl)imidazo[1,2-a]pyridine-3-carboxamide (I-526)

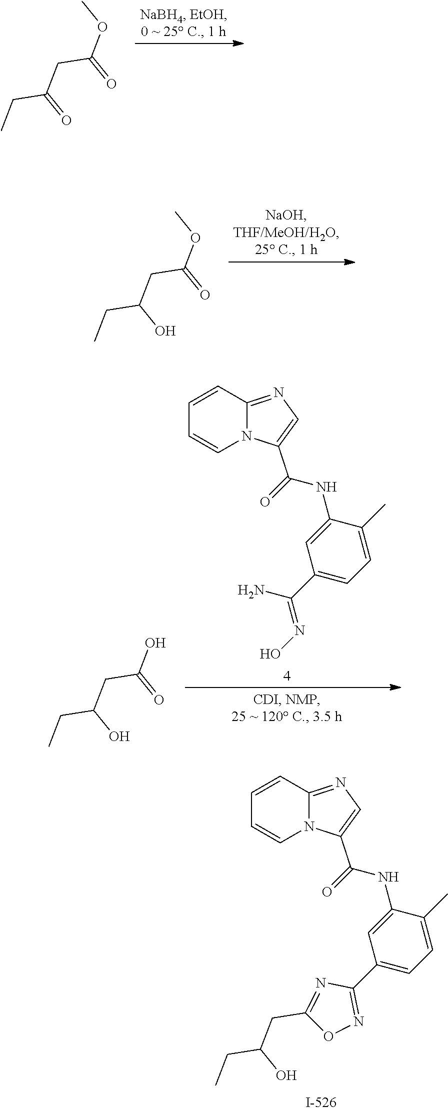

Step 1: To a solution of methyl 3-oxopentanoate (2 g, 1.0 eq) in MeOH (15 mL) was added NaBH₄ (291 mg, 0.5 eq) at 0° C. under N₂ atmosphere. The mixture was stirred at 25° C. for 1 hour under N₂ atmosphere. The reaction mixture was quenched by NH₄Cl 10 mL at 0° C., and then diluted with water (40 mL) and extracted with ethyl acetate (80 mL×3). The combined organic layers were washed with brine (50 mL×3), dried over Na₂SO₄, filtered and concentrated under reduced pressure to give a residue. The residue was purified by column chromatography (SiO₂, petroleum ether/ethyl acetate=5/1) to methyl 3-hydroxypentanoate (1.6 g, 79% yield) as a colorless oil. ¹H NMR (400 MHz, DMSO-d₆) δ=4.67 (d, J=5.6 Hz, 1H), 3.80-3.70 (m, 1H), 3.58 (s, 3H), 2.45-2.36 (m, 1H), 2.34-2.23 (m, 1H), 1.48-1.27 (m, 2H), 0.85 (t, J=7.2 Hz, 3H).

Step 2: To a solution of methyl 3-hydroxypentanoate (300 mg, 1.0 eq) in THF (1.5 mL), MeOH (1.5 mL) and H₂O (1.5 mL) was added NaOH (182 mg, 2.0 eq). The mixture was stirred at 25° C. for 1 hour. The reaction mixture was poured into water (10 mL), and then the pH of the solution was adjusted to 4˜ 5 with 1N HCl (6 mL), then the mixture was extracted with ethyl acetate (30 mL×3), dried over Na₂SO₄, filtered and concentrated under reduced pressure to give 3-hydroxypentanoic acid (240 mg, 90% yield) as a colorless oil. ¹H NMR (400 MHz, DMSO-d₆) δ=12.22-11.76 (m, 1H), 4.61 (s, 1H), 3.78-3.68 (m, 1H), 2.34-2.16 (m, 2H), 1.49-1.27 (m, 2H), 0.84 (t, J=7.2 Hz, 3H).

Step 3: To a solution of 3-hydroxypentanoic acid (100 mg, 1.0 eq) in NMP (2 mL) was added di(imidazol-1-yl)methanone (165 mg, 1.2 eq). The mixture was stirred at 25° C. for 0.5 hour. The mixture was used into the next step without further work up. Compound 3-hydroxy-1-imidazol-1-yl-pentan-1-one (140 mg, 98% yield) in NMP (2 mL) was obtained as a black liquid and used for the next step directly. To a solution of 3-hydroxy-1-imidazol-1-yl-pentan-1-one (140 mg, 1.0 eq) in NMP (2 mL) was added N-[5-[(Z)—N′-hydroxycarbamimidoyl]-2-methyl-phenyl]imidazo[1,2-a]pyridine-3-carboxamide (257 mg, 1.0 eq). The mixture was stirred at 120° C. for 3 hours. The reaction was purified directly without work-up. The residue was purified by prep-HPLC (FA condition; column: YMC-Actus Triart C18 150*30 mm*7 um; mobile phase: [water(FA)-ACN]; gradient: 23%-53% B over 10 min) to give N-(5-(5-(3-(difluoromethyl)-3-hydroxycyclobutyl)-1,2,4-oxadiazol-3-yl)-2-methylphenyl)imidazo[1,2-a]pyridine-3-carboxamide (I-526) (48.56 mg, 13% yield) as an off-white solid. ¹H NMR (400 MHz, DMSO-d₆) δ=10.02 (s, 1H), 9.46 (d, J=7.2 Hz, 1H), 8.59 (s, 1H), 8.08 (d, J=1.2 Hz, 1H), 7.85-7.75 (m, 2H), 7.56-7.46 (m, 2H), 7.18 (t, J=6.8 Hz, 1H), 5.00 (d, J=3.6 Hz, 1H), 3.91 (d, J=2.4 Hz, 1H), 3.17-3.07 (m, 1H), 3.03-2.94 (m, 1H), 2.36 (s, 3H), 1.61-1.40 (m, 2H), 0.92 (t, J=7.2 Hz, 3H). MS (ESI): m/z for C₂₁H₂₁N₅O₃ [M+H]⁺ calcd. 392.2, [M+H]+ found 392.1.

### Example 34—Preparation of N-(5-(5-((1R,2S)-2-fluorocyclopropyl)-1,2,4-oxadiazol-3-yl)-2-methylphenyl)-7-(1-methyl-1H-pyrazol-3-yl)imidazo[1,2-a]pyridine-3-carboxamide (I-529)

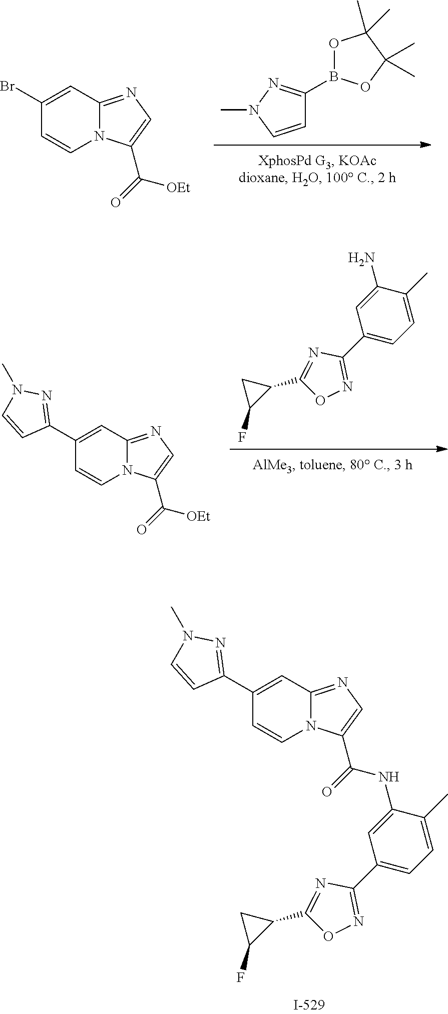

Step 1: To a solution of ethyl 7-bromoimidazo[1,2-a]pyridine-3-carboxylate (1 g, 1.0 eq) and 1-methyl-3-(4,4,5,5-tetramethyl-1,3,2-dioxaborolan-2-yl)-1H-pyrazole (1.16 g, 1.5 eq) in dioxane (12 mL) and H₂O (2 mL) was added XPhos Pd G₃ (314 mg, 0.1 eq) and K₃PO₄ (2.37 g, 3.0 eq) under N₂. The mixture was heated to 90° C. and stirred at 90° C. for 6 h under N₂. The mixture was concentrated to give a residue. The residue was purified by column chromatography (SiO₂, DCM/MeOH=10/1) to give ethyl 7-(1-methyl-1H-pyrazol-3-yl)imidazo[1,2-a]pyridine-3-carboxylate (0.9 g) as yellow solid. MS (ESI): m/z for C₁₄H₁₄N₄O₂ [M+H]⁺ calcd. 271.11, [M+H]⁺ found 271.3.

Step 2: To a solution of 5-(5-((1R,2S)-2-fluorocyclopropyl)-1,2,4-oxadiazol-3-yl)-2-methylaniline (100 mg, 1.0 eq) in toluene (1 mL) was added Al(CH₃)₃ (2 M, 536 μL, 2.5 eq). The mixture was stirred at 25° C. for 0.5 hour. To the mixture was added ethyl 7-(1-methyl-1H-pyrazol-3-yl)imidazo[1,2-a]pyridine-3-carboxylate (116 mg, 1.0 eq). The mixture was stirred at 80° C. for 3 hours. The mixture was poured into 20 mL of water and extracted with DCM (30 mL×3). The combined organic layers were washed with brine 30 mL), dried over Na₂SO₄, filtered and concentrated under reduced pressure to give a residue. The residue was purified by prep-HPLC (column: Waters Xbridge 150*25 mm*5 um; mobile phase: [water (ammonia hydroxide v/v)-ACN]; gradient: 25%-55% B over 10 min) to give N-(5-(5-((1R,2S)-2-fluorocyclopropyl)-1,2,4-oxadiazol-3-yl)-2-methylphenyl)-7-(1-methyl-1H-pyrazol-3-yl)imidazo[1,2-a]pyridine-3-carboxamide (I-529) (72.93 mg) as white solid. ¹H NMR (400 MHz, DMSO-d₆) δ=10.02 (s, 1H), 9.50-9.37 (m, 1H), 8.58 (s, 1H), 8.12 (s, 1H), 8.04 (d, J=1.6 Hz, 1H), 7.84 (d, J=2.4 Hz, 1H), 7.81-7.76 (m, 1H), 7.70-7.62 (m, 1H), 7.49 (d, J=8.0 Hz, 1H), 7.00 (d, J=2.4 Hz, 1H), 5.42-5.18 (m, 1H), 3.94 (s, 3H), 3.14-2.96 (m, 1H), 2.37 (s, 3H), 2.03-1.87 (m, 1H), 1.71-1.51 (m, 1H). MS (ESI): m/z for C24H20FN7O2 [M+H]⁺ calcd. 458.17, [M+H]⁺ found 458.2.

### Example 35—Preparation of 7-(1-methyl-1H-pyrazol-3-yl)-N-(2-methyl-5-(5-methyl-1,2,4-oxadiazol-3-yl)phenyl)imidazo[1,2-a]pyridine-3-carboxamide (I-530)

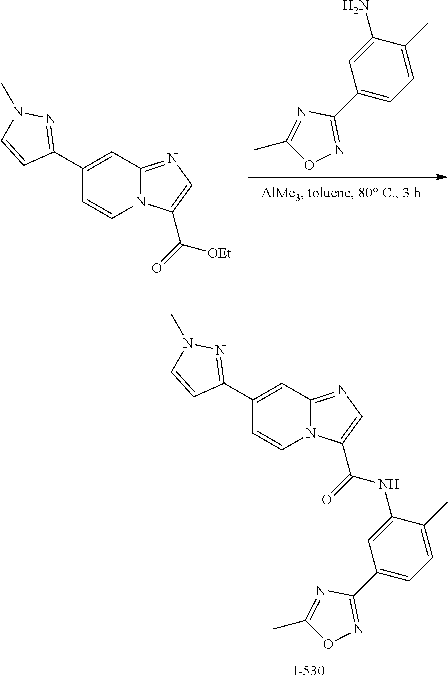

Step 1: To a solution of 2-methyl-5-(5-methyl-1,2,4-oxadiazol-3-yl) aniline(100 mg, 1.0 eq) in toluene (1 mL) was added AlMe₃ (2 M, 661 μL, 2.5 eq). The mixture was stirred at 25° C. for 0.5 hour. To the mixture was added ethyl 7-(1-methyl-1H-pyrazol-3-yl)imidazo[1,2-a]pyridine-3-carboxylate (143 mg, 1.0 eq). The mixture was stirred at 80° C. for 3 hours. The mixture was poured into 20 mL of water and extracted with DCM (30 mL×3). The combined organic layers were washed with brine (30 mL), dried over Na₂SO₄, filtered and concentrated under reduced pressure to give a residue. The residue was purified by prep-HPLC (column: Waters Xbridge 150*25 mm*5 um; mobile phase: [water (ammonia hydroxide v/v)-ACN]; gradient: 20%-50% B over 10 min) to give 7-(1-methyl-1H-pyrazol-3-yl)-N-(2-methyl-5-(5-methyl-1,2,4-oxadiazol-3-yl)phenyl)imidazo[1,2-a]pyridine-3-carboxamide (I-530) (74.5 mg) as white solid. ¹H NMR (400 MHz, DMSO-d₆) δ=10.00 (s, 1H), 9.43 (d, J=7.2 Hz, 1H), 8.59 (s, 1H), 8.13-8.10 (m, 2H), 7.86-7.78 (m, 2H), 7.71-7.62 (m, 1H), 7.49 (d, J=8.0 Hz, 1H), 6.99 (d, J=2.4 Hz, 1H), 3.94 (s, 3H), 2.67 (s, 3H), 2.38 (s, 3H). MS (ESI): m/z for C₂₂H₁₉N₇O₂ [M+H]⁺ calcd. 414.16, [M+H]⁺ found 414.2.

### Example 36—Preparation of N-(5-(5-((1R,2S)-2-fluorocyclopropyl)-1,2,4-oxadiazol-3-yl)-2-methylphenyl)-6-(1-methyl-1H-pyrazol-3-yl)imidazo[1,2-a]pyridine-3-carboxamide (I-531)

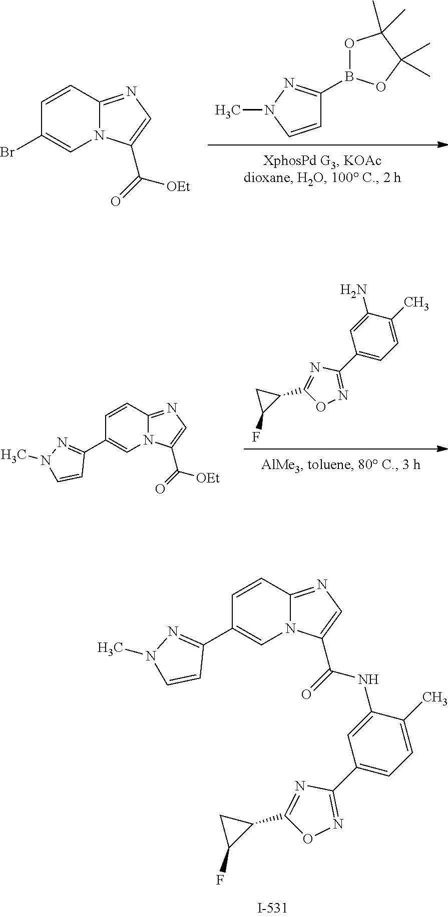

Step 1: To a solution of ethyl 6-bromoimidazo[1,2-a]pyridine-3-carboxylate (1.00 g, 1.0 eq) and ethyl 6-bromoimidazo[1,2-a]pyridine-3-carboxylate (928 mg, 1.2 eq) in dioxane (18 mL) and H₂O (3 mL) was added XPhos Pd G₃ (315 mg, 0.1 eq) and K₃PO₄ (1.97 g, 2.5 eq). The mixture was stirred at 100° C. for 2 hours. The reaction mixture was diluted with H₂O 30 mL and extracted with ethyl acetate (50 mL×3). The combined organic layers were washed with brine (25 mL×2), dried over Na₂SO₄, filtered and concentrated under reduced pressure to give a residue. The residue was purified by column chromatography (SiO₂, Petroleum ether/ethyl acetate=10/1 to 2/1) to give ethyl 6-(1-methyl-1H-pyrazol-3-yl)imidazo[1,2-a]pyridine-3-carboxylate (700 mg) as a white solid. ¹H NMR (400 MHz, METHANOL-d₄) δ=9.68 (s, 1H), 8.23 (s, 1H), 7.99 (dd, J=1.6, 9.6 Hz, 1H), 7.73 (d, J=9.6 Hz, 1H), 7.66 (d, J=2.4 Hz, 1H), 6.68 (d, J=2.4 Hz, 1H), 4.50-4.35 (m, 2H), 3.96 (s, 3H), 1.43 (t, J=7.2 Hz, 3H).

Step 2: To a solution of 5-(5-((1R,2S)-2-fluorocyclopropyl)-1,2,4-oxadiazol-3-yl)-2-methylaniline (95 mg, 1.1 eq) in toluene (1.5 mL) was added AlMe₃ (2 M, 462 μL, 2.5 eq), the reaction mixture was stirred at 25° C. for 0.5 hour, then ethyl 6-(1-methyl-1H-pyrazol-3-yl)imidazo[1,2-a]pyridine-3-carboxylate (100 mg, 1.0 eq) was added, the reaction mixture was stirred at 80° C. for 2 hours. The reaction mixture was quenched with NH₄Cl (20 mL) then was extracted with ethyl acetate (50 mL×3). The combined organic layers were washed with water (25 mL×2), dried over Na₂SO₄, filtered and concentrated under reduced pressure to give a residue. The residue was purified by prep-HPLC (FA condition; column: C18 150×30 mm; mobile phase: [water(FA)-ACN]; gradient:38%-68% B over 7 min) to give N-(5-(5-((1R,2S)-2-fluorocyclopropyl)-1,2,4-oxadiazol-3-yl)-2-methylphenyl)-6-(1-methyl-1H-pyrazol-3-yl)imidazo[1,2-a]pyridine-3-carboxamide (I-531) (110.91 mg) as a white solid. ¹H NMR (400 MHz, DMSO-d₆) δ=10.04 (s, 1H), 9.88 (s, 1H), 8.58 (s, 1H), 8.04 (d, J=1.6 Hz, 1H), 7.94 (dd, J=1.8, 9.4 Hz, 1H), 7.85-7.76 (m, 3H), 7.49 (d, J=8.0 Hz, 1H), 6.74 (d, J=2.4 Hz, 1H), 5.42-5.18 (m, 1H), 3.91 (s, 3H), 3.12-2.99 (m, 1H), 2.37 (s, 3H), 2.01-1.87 (m, 1H), 1.65-1.50 (m, 1H). MS (ESI): m/z for C₂₄H₂₀FN₇O₂[M+H]+ calcd. 458.2 [M+H]+ found 458.2.

### Example 37—Preparation of 6-(1-methyl-1H-pyrazol-3-yl)-N-(2-methyl-5-(5-methyl-1,2,4-oxadiazol-3-yl)phenyl)imidazo[1,2-a]pyridine-3-carboxamide (I-532)

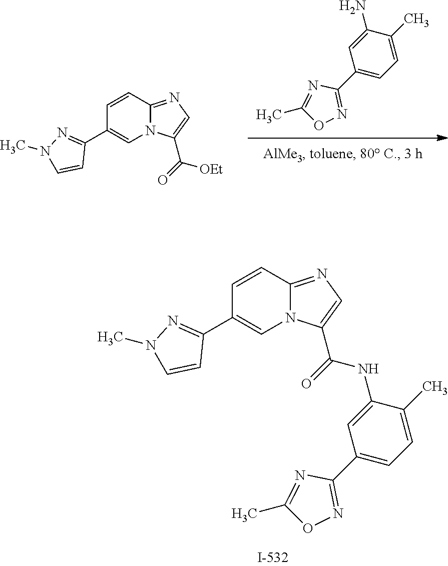

Step 1: To a solution of 2-methyl-5-(5-methyl-1,2,4-oxadiazol-3-yl) aniline (77 mg, 1.1 eq) in toluene (1.5 mL) was added AlMe₃ (2 M, 462 μL, 2.5 eq), the reaction mixture was stirred at 25° C. for 0.5 hour, then ethyl 6-(1-methyl-1H-pyrazol-3-yl)imidazo[1,2-a]pyridine-3-carboxylate (100 mg, 1.0 eq) was added, the reaction mixture was stirred at 80° C. for 2 hours. The reaction mixture was quenched with NH₄Cl (20 mL) then was extracted with ethyl acetate (50 mL×3). The combined organic layers were washed with brine (25 mL×2), dried over Na₂SO₄, filtered and concentrated under reduced pressure to give a residue. The residue was purified by prep-HPLC (FA condition; column: C18 150×30 mm; mobile phase: [water(FA)-ACN]; gradient:30%-60% B over 7 min) to give 6-(1-methyl-1H-pyrazol-3-yl)-N-(2-methyl-5-(5-methyl-1,2,4-oxadiazol-3-yl)phenyl)imidazo[1,2-a]pyridine-3-carboxamide (125.98 mg) as a white solid. ¹H NMR (400 MHz, DMSO-d₆) δ=10.02 (s, 1H), 9.89 (s, 1H), 8.59 (s, 1H), 8.11 (d, J=1.6 Hz, 1H), 7.94 (dd, J=2.0, 9.2 Hz, 1H), 7.86-7.78 (m, 3H), 7.50 (d, J=8.0 Hz, 1H), 6.75 (d, J=2.4 Hz, 1H), 3.91 (s, 3H), 2.67 (s, 3H), 2.38 (s, 3H). MS (ESI): m/z for C₂₂H₁₉N₇O₂ [M+H]+ calcd. 414.1 [M+H]+ found 414.1.

### Example 38—Preparation of 7-methoxy-N-(2-methyl-5-(5-methyl-1,2,4-oxadiazol-3-yl)phenyl)imidazo[1,2-a]pyridine-3-carboxamide (I-533)

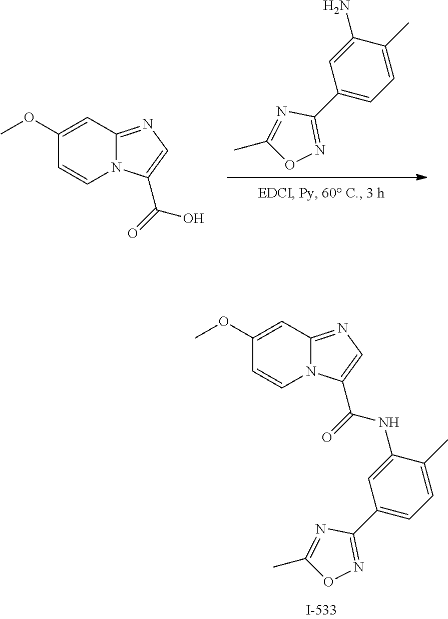

Step 1: To a solution of 7-methoxyimidazo[1,2-a]pyridine-3-carboxylic acid (50 mg, 1.0 eq) in pyridine (1.5 mL) was added EDCI (80 mg, 1.6 eq), the reaction mixture was stirred at 25° C. for 0.5 hour, then 7-methoxyimidazo[1,2-a]pyridine-3-carboxylic acid (54 mg, 1.1 eq) was added, then the reaction mixture was stirred at 60° C. for 2 hr. The reaction mixture was concentrated under reduced pressure. The residue was purified by prep-HPLC (FA condition; column: C18 150×30 mm; mobile phase: [water(FA)-ACN]; gradient:18%-48% B over 7 min) to give 7-methoxy-N-(2-methyl-5-(5-methyl-1,2,4-oxadiazol-3-yl)phenyl)imidazo[1,2-a]pyridine-3-carboxamide (29.04 mg) as a white solid. MS (ESI): m/z for C₁₉H₁₇N₅O₃, [M+H]⁺ calcd. 364.1, [M+H]⁺ found 364.1. ¹H NMR (400 MHz, METHANOL-d₄) δ=9.31 (d, J=7.8 Hz, 1H), 8.34 (s, 1H), 8.08 (d, J=1.6 Hz, 1H), 7.87 (dd, J=1.8, 8.0 Hz, 1H), 7.46 (d, J=8.0 Hz, 1H), 7.05 (d, J=2.4 Hz, 1H), 6.85 (dd, J=2.4, 7.6 Hz, 1H), 3.95 (s, 3H), 2.65 (s, 3H), 2.40 (s, 3H).

### Example 39—Preparation of 6-(1-methyl-1H-pyrazol-3-yl)-N-(2-methyl-5-(5-methyl-1,2,4-oxadiazol-3-yl)phenyl)pyrazolo[1,5-a]pyridine-3-carboxamide (I-535)

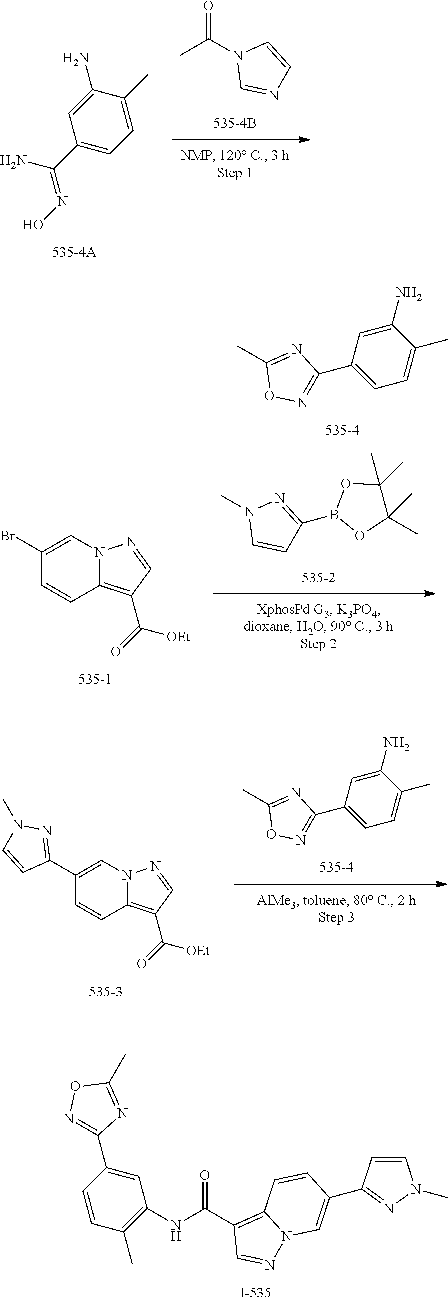

Step 1: A solution of 535-4B (2 g, 1.0 eq) and 535-4A (3.00 g, 1.0 eq) in NMP (10 mL) was stirred at 25° C. for 5 minutes. The mixture was heated at 120° C. for 2 hours. The mixture was poured into 20 mL of water and extracted with ethyl acetate (30 mL×3). The combined organic layers were washed with brine (20 mL×3), dried over Na₂SO₄, filtered and concentrated under reduced pressure to give a residue. The residue was purified by column chromatography (SiO₂, petroleum ether/ethyl acetate=3/1) to give 2-methyl-5-(5-methyl-1,2,4-oxadiazol-3-yl) aniline (1.6 g) as yellow solid. ¹H NMR (400 MHz, chloroform-d) δ=7.45-7.34 (m, 2H), 7.15 (d, J=7.6 Hz, 1H), 3.98-3.51 (s, 2H), 2.63 (s, 3H), 2.21 (s, 3H). MS (ESI): m/z for C₁₀H₁₁N₃O [M+H]⁺ calcd. 190.09 [M+H]⁺ found. 190.3

Step 2: To a solution of 535-1 (500 mg, 1.0 eq) and 535-2 (464 mg, 1.2 eq) in dioxane (6 mL) and H₂O (1 mL) was added XPhos Pd G₃ (157 mg, 0.1 eq) and K₃PO₄ (1.18 g, 3.0 eq) under N₂. The mixture was stirred at 90° C. for 3 h under N₂. The mixture was poured into 20 mL of water and extracted with ethyl acetate (30 mL×3). The combined organic layers were washed with brine 30 mL, dried over Na₂SO₄, filtered and concentrated under reduced pressure to give a residue. The residue was purified by column chromatography (SiO₂, petroleum ether/ethyl acetate=3/1) to give ethyl 6-(1-methyl-1H-pyrazol-3-yl)pyrazolo[1,5-a]pyridine-3-carboxylate (350 mg) as white solid. ¹H NMR (400 MHz, chloroform-d) δ=8.91 (s, 1H), 8.40 (s, 1H), 8.21-8.14 (m, 1H), 7.95-7.87 (m, 1H), 7.44 (d, J=2.4 Hz, 1H), 6.56 (d, J=2.4 Hz, 1H), 4.46-4.34 (m, 2H), 3.98 (s, 3H), 1.43 (t, J=7.2 Hz, 3H). MS (ESI): m/z for C₁₄H₁₄N₄O₂ [M+H]⁺ calcd. 271.11 [M+H]⁺ found. 271.3

Step 3: To a solution of 535-4 (100 mg, 1.0 eq) in toluene (2 mL) was added AlMe₃ (2 M, 661 μL, 2.5 eq). The mixture was stirred at 25° C. for 0.5 hour. To the mixture was added ethyl 6-(1-methyl-1H-pyrazol-3-yl)pyrazolo[1,5-a]pyridine-3-carboxylate (143 mg, 1.0 eq). The mixture was stirred at 80° C. for 2 hours. The mixture was poured into 10 mL of water and extracted with DCM (20 mL×3). The combined organic layers were washed with brine 20 mL, dried over Na₂SO₄, filtered and concentrated under reduced pressure to give a residue. The residue was purified by prep-HPLC (column: C18 150×30 mm; mobile phase: [water(FA)-ACN]; gradient: 40%-70% B over 7 min) to give 6-(1-methyl-1H-pyrazol-3-yl)-N-(2-methyl-5-(5-methyl-1,2,4-oxadiazol-3-yl)phenyl)pyrazolo[1,5-a]pyridine-3-carboxamide (I-535, 61.43 mg) as white solid. ¹H NMR (400 MHz, DMSO-d₆) δ=9.74 (s, 1H), 9.21 (s, 1H), 8.78 (s, 1H), 8.26 (d, J=9.2 Hz, 1H), 8.11 (d, J=1.2 Hz, 1H), 8.04-7.97 (m, 1H), 7.80 (d, J=2.0 Hz, 1H), 7.79-7.74 (m, 1H), 7.46 (d, J=8.0 Hz, 1H), 6.92 (d, J=2.0 Hz, 1H), 3.91 (s, 3H), 2.66 (s, 3H), 2.36 (s, 3H). MS (ESI): m/z for C22H19N7O2 [M+H]⁺ calcd. 414.16 [M+H]⁺ found. 414.3

### Example 40—Preparation of 7-methoxy-N-(2-methyl-5-(5-(pyridin-2-yl)-1,2,4-oxadiazol-3-yl)phenyl)imidazo[1,2-a]pyridine-3-carboxamide (I-536)

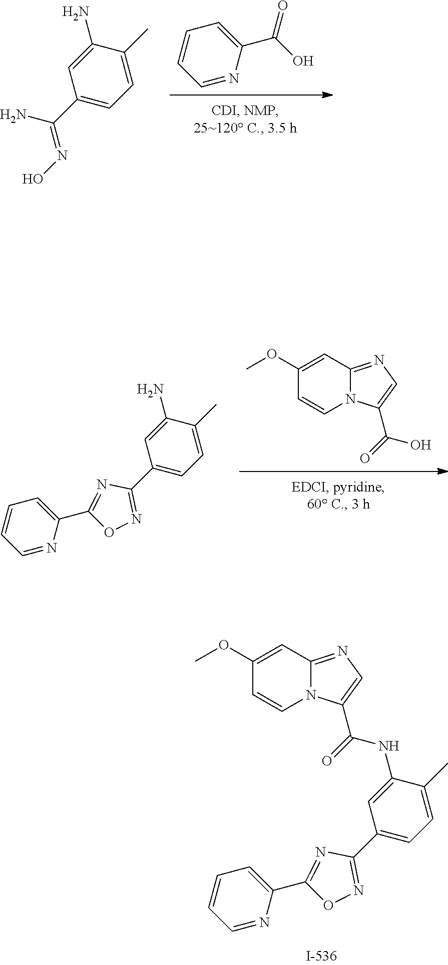

Step 1: To a solution of pyridine-2-carboxylic acid (500 mg, 1.0 eq) in NMP (5 mL) was added di(imidazol-1-yl)methanone (790 mg, 1.2 eq). The mixture was stirred at 25° C. for 0.5 hour. The mixture was used into the next step without further work up. Compound imidazol-1-yl(2-pyridyl)methanone (700 mg, crude) in NMP (5 mL) was obtained as a red liquid and used directly. To a solution of imidazol-1-yl(2-pyridyl)methanone (700 mg, 4.04 mmol, 1.0 eq) in NMP (5 mL) was added 3-amino-N′-hydroxy-4-methyl-benzamidine (668 mg, 1.0 eq). The mixture was stirred at 120° C. for 3 hours. The reaction mixture was diluted with water (15 mL) and extracted with ethyl acetate (30 mL×3). The combined organic layers were washed with brine (30 mL×3), dried over Na₂SO₄, filtered and concentrated under reduced pressure to give 2-methyl-5-(5-(pyridin-2-yl)-1,2,4-oxadiazol-3-yl) aniline (1 g, crude) as a yellow solid. ¹H NMR (400 MHz, DMSO-d₆) δ=8.84 (d, J=4.4 Hz, 1H), 8.29 (d, J=8.0 Hz, 1H), 8.11 (t, J=7.2 Hz, 1H), 7.75-7.69 (m, 1H), 7.40 (s, 1H), 7.22 (d, J=7.6 Hz, 1H), 7.12 (d, J=7.6 Hz, 1H), 5.22 (s, 2H), 2.13 (s, 3H). MS (ESI): m/z for C₁₄H₁₂N₄O [M+H]⁺ calcd. 253.10, [MH]⁺ found. 253.1.

Step 2: To a solution of 2-methyl-5-[5-(2-pyridyl)-1,2,4-oxadiazol-3-yl]aniline (100 mg, 1.0 eq) in pyridine (1 mL) was added EDCI (122 mg, 1.6 eq) and 7-methoxyimidazo[1,2-a]pyridine-3-carboxylic acid (76 mg, 1.0 eq). The mixture was stirred at 60° C. for 3 hours. The reaction mixture was diluted with water (10 mL) and extracted with ethyl acetate (20 mL×3). The combined organic layers were washed with brine (20 mL×3), dried over Na₂SO₄, filtered and concentrated under reduced pressure to give a residue. The residue was purified by prep-HPLC (FA condition; column: YMC-Actus Triart C18 150*30 mm*7 um; mobile phase: [water(FA)-ACN]; gradient: 20%-50% B over 10 min) to give 7-methoxy-N-(2-methyl-5-(5-(pyridin-2-yl)-1,2,4-oxadiazol-3-yl)phenyl)imidazo[1,2-a]pyridine-3-carboxamide (I-536, 6.46 mg, 3% yield) as a white solid. ¹H NMR (400 MHz, DMSO-d₆) δ=9.94 (s, 1H), 9.28 (d, J=7.6 Hz, 1H), 8.86 (d, J=4.0 Hz, 1H), 8.47 (s, 1H), 8.35 (d, J=7.6 Hz, 1H), 8.18 (d, J=1.2 Hz, 1H), 8.16-8.10 (m, 1H), 7.94-7.89 (m, 1H), 7.77-7.72 (m, 1H), 7.54 (d, J=8.0 Hz, 1H), 7.17 (d, J=2.4 Hz, 1H), 6.90-6.86 (m, 1H), 3.90 (s, 3H), 2.38 (s, 3H). MS (ESI): m/z for C₂₃H₁₈N₆O₃ [M+H]⁺ calcd. 427.14, [MH]⁺ found. 427.1.

### Example 41—Preparation of 7-methoxy-N-(2-methyl-5-(5-(4-methylbenzyl)-1,2,4-oxadiazol-3-yl)phenyl)imidazo[1,2-a]pyridine-3-carboxamide (I-537)

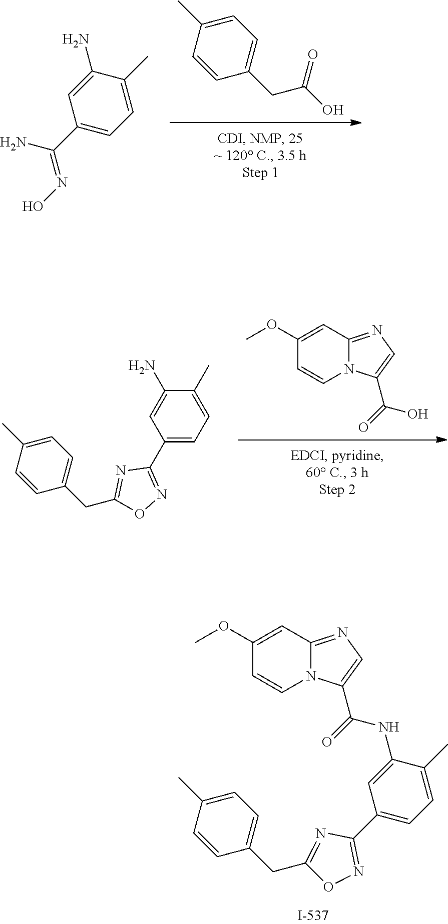

Step 1: To a solution of 2-(p-tolyl)acetic acid (500 mg, 1.0 eq) in NMP (5 mL) was added di(imidazol-1-yl)methanone (648 mg, 1.2 eq). The mixture was stirred at 25° C. for 0.5 hours. 1-imidazol-1-yl-2-(p-tolyl)ethanone (666 mg, crude) in NMP (5 mL) was obtained as a red liquid and used directly. To a solution of 1-imidazol-1-yl-2-(p-tolyl)ethanone (666 mg, 1.0 eq) in NMP (5 mL) was added 3-amino-N′-hydroxy-4-methyl-benzamidine (549 mg, 1.0 eq). The mixture was stirred at 120° C. for 3 hours. The reaction mixture was diluted with water (15 mL) and extracted with ethyl acetate (30 mL×3). The combined organic layers were washed with brine (30 mL×3), dried over Na₂SO₄, filtered and concentrated under reduced pressure to give 2-methyl-5-(5-(4-methylbenzyl)-1,2,4-oxadiazol-3-yl) aniline (830 mg, crude) as a yellow solid. ¹H NMR (400 MHz, DMSO-d₆) δ=7.31-6.99 (m, 9H), 4.33 (s, 2H), 2.28 (s, 3H), 2.09 (s, 3H). MS (ESI): m/z for C₁₇H₁₇N₃O [M+H]⁺ calcd. 280.14, [MH]⁺ found. 280.1.

Step 2: To a solution of 2-methyl-5-[5-(p-tolylmethyl)-1,2,4-oxadiazol-3-yl]aniline (150 mg, 1.0 eq) in pyridine (1.5 mL) was added EDCI (165 mg, 1.6 eq) and 7-methoxyimidazo[1,2-a]pyridine-3-carboxylic acid (103 mg, 1.0 eq). The mixture was stirred at 60° C. for 3 hours. The reaction mixture was diluted with water (10 mL) and extracted with ethyl acetate (20 mL×3). The combined organic layers were washed with brine (20 mL×3), dried over Na₂SO₄, filtered and concentrated under reduced pressure to give a residue. The residue was purified by prep-HPLC (FA condition; column: YMC-Actus Triart C18 150*30 mm*7 um; mobile phase: [water(FA)-ACN]; gradient:33%-63% B over 10 min) to give: 7-methoxy-N-(2-methyl-5-(5-(4-methylbenzyl)-1,2,4-oxadiazol-3-yl)phenyl)imidazo[1,2-a]pyridine-3-carboxamide (I-537, 37.39 mg, 14% yield) as an off-white solid. ¹H NMR (400 MHz, DMSO-d₆) δ=9.89 (s, 1H), 9.25 (d, J=7.6 Hz, 1H), 8.44 (s, 1H), 8.02 (d, J=1.2 Hz, 1H), 7.80-7.75 (m, 1H), 7.46 (d, J=8.0 Hz, 1H), 7.27 (d, J=8.0 Hz, 2H), 7.19-7.14 (m, 3H), 6.89-6.84 (m, 1H), 4.37 (s, 2H), 3.89 (s, 3H), 2.34 (s, 3H), 2.28 (s, 3H). MS (ESI): m/z for C₂₆H₂₃N₅O₃ [M+H]⁺ calcd. 454.18, [MH]+ found. 454.1.

### Example 42—Preparation of 4 N-(5-(5-(4,4-difluorocyclohexyl)-1,2,4-oxadiazol-3-yl)-2-methylphenyl)-7-methylimidazo[1,2-a]pyridine-3-carboxamide (I-540)

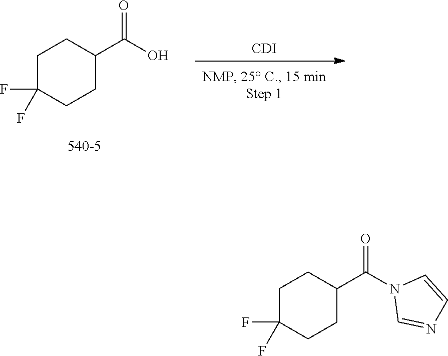 [SMILES: O=C(O)C1CCC(F)(F)CC1 ; "CDI" ; O=C(C1CCC(F)(F)CC1)n1ccnc1]

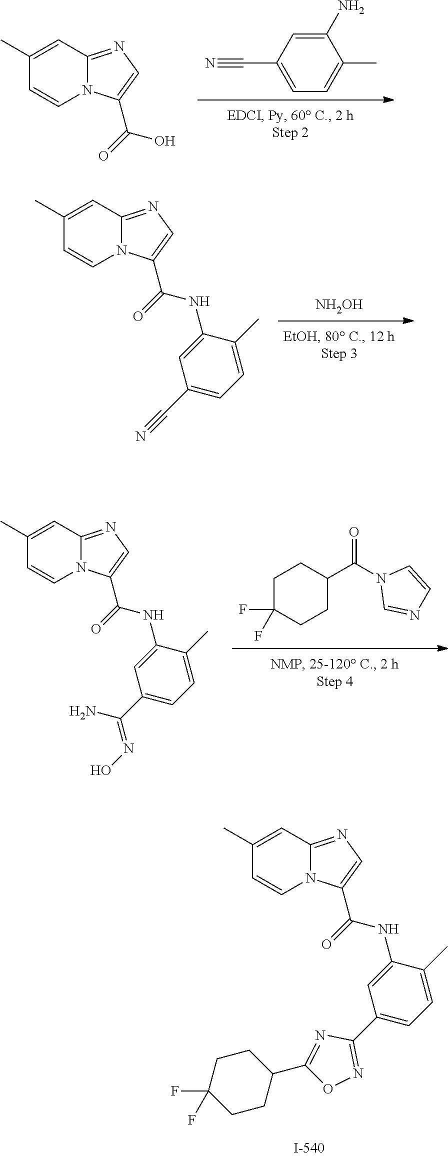

Step 1: To a solution of 540-5 (60 mg, 1.0 eq) in NMP (1 mL) was added CDI (72 mg, 1.2 eq). The mixture was stirred at 25° C. for 15 minutes. (4,4-difluorocyclohexyl)(1H-imidazol-1-yl)methanone (78 mg, crude) in NMP was used without further work-up.

Step 2: To a solution of 3-amino-4-methyl-benzonitrile (0.5 g, 1.0 eq) and 7-methylimidazo[1,2-a]pyridine-3-carboxylic acid (952 mg, 1.0 eq) in pyridine (5 mL) was added EDCI (870 mg, 1.2 eq). The mixture was stirred at 60° C. for 2 hours. The mixture was cooled to rt. The mixture was poured into 200 mL of water. Off-white solids were separated out and the solid was washed with 50 mL of water and 5 mL of EtOH. The white solid was dried under high vacuum. N-(5-cyano-2-methylphenyl)-7-methylimidazo[1,2-a]pyridine-3-carboxamide (0.5 g, 45% yield) was obtained as an off-white solid. MS (ESI): m/z for: C₁₇H₁₄N₄O [M+H]⁺ calcd. 291.12 [M+H]⁺ found: 291.3

Step 3: To a solution of N-(5-cyano-2-methylphenyl)-7-methylimidazo[1,2-a]pyridine-3-carboxamide (2.9 g, 1.0 eq) in EtOH (30 mL) was added hydroxylamine (1.32 g, 12 eq). The mixture was stirred at 80° C. for 12 hours. The mixture was concentrated to give N-(5-cyano-2-methylphenyl)-7-methylimidazo[1,2-a]pyridine-3-carboxamide (3.19 μg, crude) as white solid. MS (ESI): m/z for: C₁₇H₁₇N₅O₂ [M+H]⁺ calcd. 324.14[M+H]⁺ found. 324.3

Step 4: To (4,4-difluorocyclohexyl)(1H-imidazol-1-yl)methanone (78 mg, 1.2 eq) in NMP (1 mL) was added (Z)—N-(5-(N′-hydroxycarbamimidoyl)-2-methylphenyl)-7-methylimidazo[1,2-a]pyridine-3-carboxamide (100 mg, 1.0 eq). The mixture was stirred at 25° C. for 5 minutes. The mixture was heated to 120° C. and stirred at 120° C. for 1 hour. The mixture was filtered to give a clear solution. The solution was purified by prep-HPLC (column: C18 150×30 mm; mobile phase: [water(FA)-ACN]; gradient:35%-65% B over 7 min) to give 4 N-(5-(5-(4,4-difluorocyclohexyl)-1,2,4-oxadiazol-3-yl)-2-methylphenyl)-7-methylimidazo[1,2-a]pyridine-3-carboxamide (I-540, 68.19 mg, 44% yield) as white solid. MS (ESI): m/z for: C24H23F2N5O2 [M+H]⁺ calcd. 452.18[M+H]⁺ found: 452.4. ¹H NMR (400 MHz, DMSO-d₆) δ=9.96 (s, 1H), 9.32 (d, J=7.2 Hz, 1H), 8.51 (s, 1H), 8.06 (s, 1H), 7.80 (d, J=8.0 Hz, 1H), 7.56 (s, 1H), 7.48 (d, J=8.0 Hz, 1H), 7.02 (d, J=7.6 Hz, 1H), 2.42 (s, 3H), 2.35 (s, 3H), 2.26-1.77 (m, 8H).

### Example 43—Preparation of N-(5-(5-((1R,2S)-2-fluorocyclopropyl)-1,2,4-oxadiazol-3-yl)-2-methylphenyl)-6-(thiazol-4-yl)imidazo[1,2-a]pyridine-3-carboxamide (I-541)

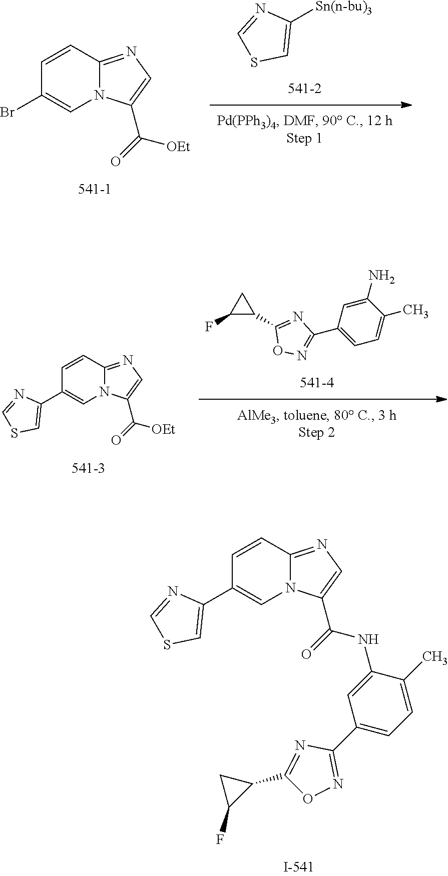

Step 1: To a solution of 541-1 (300 mg, 1.0 eq) and 542-2 (460 mg, 1.1 eq) in DMF (3 mL) was added Pd(PPh₃)₄ (130 mg, 0.1 eq). The mixture was stirred at 90° C. for 12 hours under N₂. The reaction mixture was diluted with H₂O (50 mL) and extracted with ethyl acetate (40 mL×3). The combined organic layers were washed with brine (30 mL×2), dried over Na₂SO₄, filtered and concentrated under reduced pressure to give a residue. The residue was purified by column chromatography (SiO₂, petroleum ether/ethyl acetate=10/1 to 1/1) to give ethyl 6-(thiazol-4-yl)imidazo[1,2-a]pyridine-3-carboxylate (180 mg) as a white solid. ¹H NMR (400 MHz, methanol-d₄) δ=9.96 (s, 1H), 9.11 (d, J=1.6 Hz, 1H), 8.26 (s, 1H), 8.12 (dd, J=1.2, 9.2 Hz, 1H), 8.05 (d, J=1.6 Hz, 1H), 7.77 (d, J=9.2 Hz, 1H), 4.45 (q, J=7.2 Hz, 2H), 1.44 (t, J=7.2 Hz, 3H).

Step 2: To a solution of 541-4 (38 mg, 1.1 eq) in toluene (1 mL) was added AlMe₃ (2 M, 183 μL, 2.5 eq), then the reaction mixture was stirred at 25° C. for 0.5 hour. Then ethyl 6-(thiazol-4-yl)imidazo[1,2-a]pyridine-3-carboxylate (40 mg, 1.0 eq) was added, the reaction mixture was stirred at 80° C. for 2 hours. The reaction mixture was quenched with NH₄Cl (20 mL) then was extracted with DCM (50 mL×3). The combined organic layers were washed with water (25 mL×2), dried over Na₂SO₄, filtered and concentrated under reduced pressure to give a residue. The residue was purified by prep-HPLC (FA condition; column: Phenomenex luna C18 150×25 mm×10 um; mobile phase: [water(FA)-ACN]; gradient:388%-68% B over 9 min) to give N-(5-(5-((1R,2S)-2-fluorocyclopropyl)-1,2,4-oxadiazol-3-yl)-2-methylphenyl)-6-(thiazol-4-yl)imidazo[1,2-a]pyridine-3-carboxamide (I-541, 26.97 mg) as a white solid. ¹H NMR (400 MHz, methanol-d₄) δ=10.17 (d, J=0.8 Hz, 1H), 9.09 (d, J=2.0 Hz, 1H), 8.49 (s, 1H), 8.16-8.12 (m, 1H), 8.11 (d, J=1.6 Hz, 1H), 8.04 (d, J=2.0 Hz, 1H), 7.86 (dd, J=2.0, 8.0 Hz, 1H), 7.80 (dd, J=0.8, 9.2 Hz, 1H), 7.46 (d, J=8.0 Hz, 1H), 5.25-5.01 (m, 1H), 2.97-2.78 (m, 1H), 2.43 (s, 3H), 1.96-1.79 (m, 1H), 1.69-1.54 (m, 1H). MS (ESI): m/z for C₂₃H₁₇FN₆O₂S [M+H]+ calcd. 461.0 [M+H]+ found: 461.0.

### Example 44—Preparation of N-(2-methyl-5-(5-methyl-1,2,4-oxadiazol-3-yl)phenyl)-6-(thiazol-4-yl)imidazo[1,2-a]pyridine-3-carboxamide (I-542)

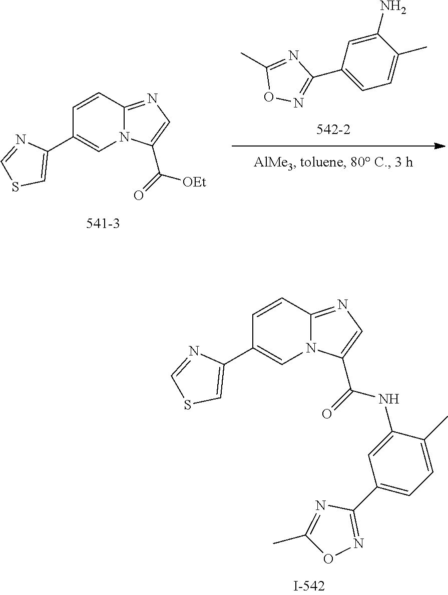

To a solution of 542-2 (30 mg, 1.1 eq) in toluene (1 mL) was added AlMe₃ (2 M, 182.94 L, 2.5 eq), the reaction mixture was stirred at 25° C. for 0.5 hour, then 541-3 (40 mg, 146.35 μmol, 1.0 eq) was added and the reaction mixture was stirred at 80° C. for 2 hours. The reaction mixture was quenched with NH₄Cl (20 mL) and then was extracted with DCM (50 mL×3). The combined organic layers were washed with water (25 mL×2), dried over Na₂SO₄, filtered and concentrated under reduced pressure to give a residue. The residue was purified by prep-HPLC (FA condition; column: Phenomenex luna C18 150×25 mm×10 um; mobile phase: [water(FA)-ACN]; gradient:32%-62% B over 9 min) to give N-(2-methyl-5-(5-methyl-1,2,4-oxadiazol-3-yl)phenyl)-6-(thiazol-4-yl)imidazo[1,2-a]pyridine-3-carboxamide (I-542, 15.66 mg) as a white solid. ¹H NMR (400 MHz, methanol-d₄) δ=10.18 (s, 1H), 9.09 (d, J=2.0 Hz, 1H), 8.50 (s, 1H), 8.16-8.12 (m, 2H), 8.05 (d, J=2.0 Hz, 1H), 7.89 (dd, J=1.6, 8.0 Hz, 1H), 7.81 (dd, J=0.8, 9.6 Hz, 1H), 7.48 (d, J=8.0 Hz, 1H), 2.66 (s, 3H), 2.44 (s, 3H). MS (ESI): m/z for C₂₁H₁₆N₆O₂S [M+H]+ calcd. 417.1 [M+H]⁺ found: 417.1

### Example 45—Preparation of N-(5-(5-((1R,2S)-2-fluorocyclopropyl)-1,2,4-oxadiazol-3-yl)-2-methylphenyl)-7-(thiazol-4-yl)imidazo[1,2-a]pyridine-3-carboxamide (I-547)

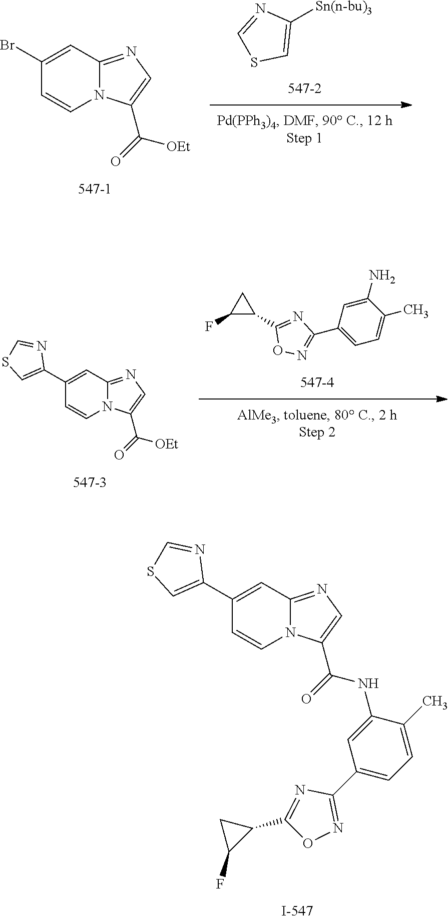

Step 1: To a solution of 547-1 (300 mg, 1.0 eq) in DMF (5 mL) was added 547-2 (459 mg, 1.1 eq) and Pd(PPh₃)₄ (129 mg, 0.1 eq). The mixture was stirred at 90° C. for 12 hours. The reaction mixture was quenched by the addition saturated aqueous potassium fluoride solution (40 mL) at 0° C., and then diluted with H₂O (40 mL) and extracted with ethyl acetate (100 mL×3). The combined organic layers were washed with brine (50 mL×3), dried over Na₂SO₄, filtered and concentrated under reduced pressure to give a residue. The residue was purified by column chromatography (SiO₂, petroleum ether/ethyl acetate=1/1 to 0.8/1) to give ethyl 7-(thiazol-4-yl)imidazo[1,2-a]pyridine-3-carboxylate (90 mg) as a white solid. H NMR (400 MHz, DMSO-d₆) δ=9.30 (d, J=1.6 Hz, 1H), 9.26 (d, J=7.2 Hz, 1H), 8.56 (d, J=1.6 Hz, 1H), 8.38 (s, 1H), 8.33 (s, 1H), 7.90 (dd, J=1.6, 7.2 Hz, 1H), 4.45-4.32 (m, 2H), 1.36 (t, J=7.2 Hz, 3H)

Step 2: To a solution of 547-4 (37 mg, 1.1 eq) in toluene (1 mL) was added Al(CH₃)₃ (2 M, 2.5 eq). The mixture was stirred at 25° C. for 0.5 hour. Then 547-3 (40 mg, 1.0 eq) was added. The mixture was stirred at 80° C. for 1.5 hours. The reaction mixture was quenched by the addition of a saturated solution of ammonium chloride in water (20 mL) at 0° C., and then diluted with H₂O (20 mL) and extracted with DCM (20 mL×3). The combined organic layers were washed with brine (15 mL×2), dried over Na₂SO₄ filtered and concentrated under reduced pressure to give a residue. The residue was purified by prep-HPLC (column: C18 150×30 mm; mobile phase: [water(FA)-ACN]; gradient: 38%-68% B over 7 min) to give N-(5-(5-((1R,2S)-2-fluorocyclopropyl)-1,2,4-oxadiazol-3-yl)-2-methylphenyl)-7-(thiazol-4-yl)imidazo[1,2-a]pyridine-3-carboxamide (I-547, 20.84 mg) as a white solid. ¹H NMR (400 MHz, DMSO-d6) δ=10.05 (s, 1H), 9.48 (d, J=7.2 Hz, 1H), 9.30 (d, J=2.0 Hz, 1H), 8.62 (s, 1H), 8.54 (d, J=2.0 Hz, 1H), 8.35 (s, 1H), 8.04 (d, J=1.6 Hz, 1H), 7.85-7.75 (m, 2H), 7.49 (d, J=8.0 Hz, 1H), 5.42-5.16 (m, 1H), 3.11-3.00 (m, 1H), 2.37 (s, 3H), 2.03-1.86 (m, 1H), 1.65-1.53 (m, 1H). MS (ESI): m/z for C₂₃H₁₇FN₆O₂S [M+H]⁺ calcd. 461.5, [MH]⁺ found: 461.2.

### Example 46—Preparation of N-(2-methyl-5-(5-methyl-1,2,4-oxadiazol-3-yl)phenyl)-7-(thiazol-4-yl)imidazo[1,2-a]pyridine-3-carboxamide (I-548)

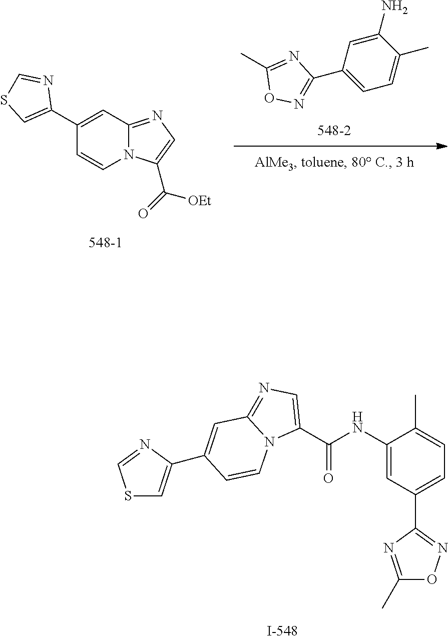

To a solution of 548-2 (30.46 mg, 1.1 eq) in toluene (1 mL) was added Al(CH₃)₃ (2 M, 2.5 eq). The mixture was stirred at 25° C. for 0.5 hour. Then 548-1 (40 mg, 1.0 eq) was added and the mixture was stirred at 80° C. for 1.5 hours. The reaction mixture was quenched by the addition of saturated ammonium chloride in water (20 mL) at 0° C., and then diluted with H₂O (20 mL) and extracted with DCM (20 mL×3). The combined organic layers were washed with brine (15 mL×2), dried over Na₂SO₄ filtered and concentrated under reduced pressure to give a residue. The residue was purified by prep-HPLC (column: C18 150×30 mm; mobile phase: [water(FA)-ACN]; gradient:28%-58% B over 7 min) to give N-(2-methyl-5-(5-methyl-1,2,4-oxadiazol-3-yl)phenyl)-7-(thiazol-4-yl)imidazo[1,2-a]pyridine-3-carboxamide (I-548, 19.97 mg) as a white solid. ¹H NMR (400 MHz, DMSO-d₆) δ=10.04 (s, 1H), 9.53-9.46 (m, 1H), 9.30 (d, J=2.0 Hz, 1H), 8.62 (s, 1H), 8.54 (d, J=2.0 Hz, 1H), 8.36 (d, J=0.8 Hz, 1H), 8.10 (d, J=1.6 Hz, 1H), 7.88-7.76 (m, 2H), 7.50 (d, J=8.0 Hz, 1H), 2.67 (s, 3H), 2.38 (s, 3H). MS (ESI): m/z for C₁₀H₁₁N₃O [M+H]⁺ calcd. 417.5, [MH]⁺ found. 417.2.

### Example 47—Preparation of 7-methyl-N-(2-methyl-5-(5-(4-methylpyridin-2-yl)-1,2,4-oxadiazol-3-yl)phenyl)imidazo[1,2-a]pyridine-3-carboxamide (I-549)

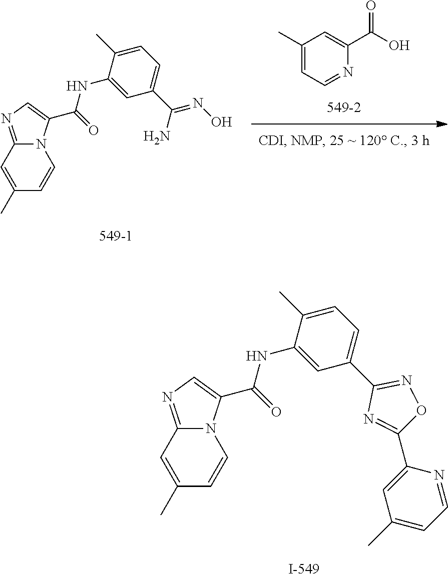

To a solution of 549-2 (0.1 g, 1.0 eq) in NMP (2 mL) was added CDI (141 mg, 1.2 eq). The mixture was stirred at 25° C. for 0.5 hour. The mixture was used directly. To the solution was added 549-1 (235 mg, 1.0 eq). The mixture was stirred at 120° C. for 2.5 hours. The mixture was purified directly by prep-HPLC (column: Phenomenex luna C18 150*40 mm*15 um; mobile phase: [water(FA)-ACN]; gradient:20%-50% B over 15 min) to give 7-methyl-N-(2-methyl-5-(5-(4-methylpyridin-2-yl)-1,2,4-oxadiazol-3-yl)phenyl)imidazo[1,2-a]pyridine-3-carboxamide (I-549, 139.12 mg) as a white solid. ¹H NMR (400 MHz, DMSO-d₆) δ=9.98 (s, 1H), 9.35 (d, J=7.2 Hz, 1H), 8.71 (d, J=5.2 Hz, 1H), 8.55 (s, 1H), 8.22 (dd, J=1.2, 3.2 Hz, 2H), 7.92 (dd, J=1.6, 8.0 Hz, 1H), 7.61-7.52 (m, 3H), 7.04 (dd, J=1.6, 7.2 Hz, 1H), 2.49 (s, 3H), 2.44 (s, 3H), 2.40 (s, 3H). MS (ESI): m/z for C₂₄H₂₀N₆O₂ [M+H]⁺ calcd. 425.5, [MH]⁺ found: 425.2.

### Example 48—Preparation of N-(5-(5-benzyl-1,2,4-oxadiazol-3-yl)-2-methylphenyl)-7-methoxyimidazo[1,2-a]pyridine-3-carboxamide (I-552)

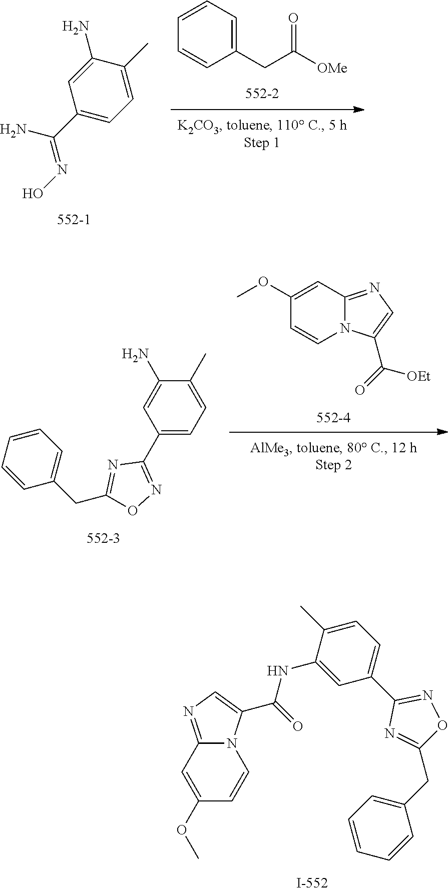

Step 1: A mixture of 3-amino-N′-hydroxy-4-methyl-benzamidine (200 mg, 1.0 eq), methyl 2-phenylacetate (273 mg, 1.5 eq), and K₂CO₃ (502 mg, 3.0 eq) in toluene (10 mL) was stirred at 110° C. for 5 hours. The mixture was concentrated to remove the solvent to give a residue. The residue was purified by column chromatography (SiO₂, petroleum ether/ethyl acetate=10/1 to 2/1) to give 5-(5-benzyl-1,2,4-oxadiazol-3-yl)-2-methylaniline (230 mg, 72% yield) as a white solid. ¹H NMR (400 MHz, DMSO-d₆) δ=7.44-7.21 (m, 6H), 7.11-7.02 (m, 2H), 5.15 (s, 2H), 4.39 (s, 2H), 2.09 (s, 3H). MS (ESI): m/z for C₁₆H₁₅N₃O [M+H]⁺ calcd. 266.12, [M+H]⁺ found: 266.1.

Step 2: To a mixture of 552-3 (60 mg, 1.0 eq) in toluene (1 mL) was added AlMe₃ (2 M, 2.5 eq) and the mixture was stirred at 25° C. for 0.5 hour under N₂. Then 552-4 (55 mg, 1.1 eq) was added and the resultant mixture was stirred at 80° C. for 12 hours under N₂. The reaction mixture was quenched with NH₄Cl (10 mL) and then was extracted with DCM (20 mL×3). The combined organic layers were washed with brine (15 mL×2), dried over Na₂SO₄, filtered and concentrated under reduced pressure to give a residue. The residue was purified by prep-HPLC (column: C18 150×30 mm; mobile phase: [water(FA)-ACN]; gradient:30%-60% B over 7 min) to give N-(5-(5-benzyl-1,2,4-oxadiazol-3-yl)-2-methylphenyl)-7-methoxyimidazo[1,2-a]pyridine-3-carboxamide (1-552, 45 mg, 43% yield) as a white solid. ¹H NMR (400 MHz, DMSO-d₆) δ=9.89 (s, 1H), 9.26 (d, J=7.6 Hz, 1H), 8.44 (s, 1H), 8.03 (d, J=0.8 Hz, 1H), 7.82-7.74 (m, 1H), 7.47 (d, J=8.0 Hz, 1H), 7.43-7.35 (m, 4H), 7.34-7.26 (m, 1H), 7.16 (d, J=2.4 Hz, 1H), 6.86 (dd, J=2.4, 7.6 Hz, 1H), 4.44 (s, 2H), 3.89 (s, 3H), 2.34 (s, 3H). MS (ESI): m/z for C₂₅H₂₁N₅O₃ [M+H]⁺ calcd. 440.16, [M+H]⁺ found: 440.2.

### Example 49—Preparation of 7-methyl-N-(2-methyl-5-(5-((tetrahydrofuran-2-yl)methyl)-1,2,4-oxadiazol-3-yl)phenyl)imidazo[1,2-a]pyridine-3-carboxamide (I-553)

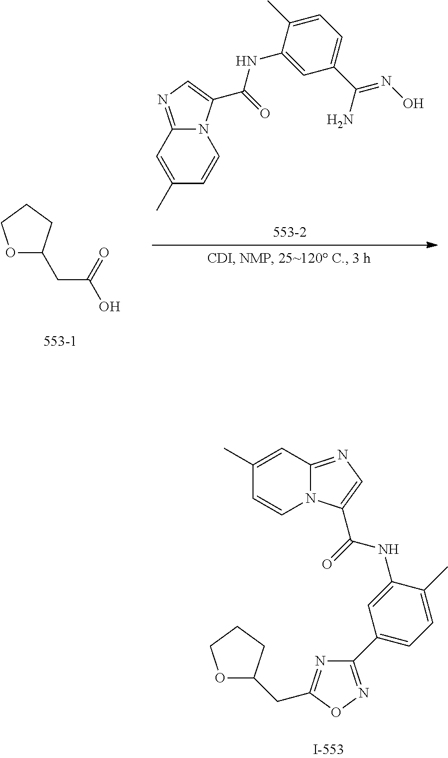 [SMILES: O=C(O)CC1CCCO1 ; Cc1ccn2c(C(=O)Nc3cc(/C(N)=N/O)ccc3C)cnc2c1 ; Cc1ccn2c(C(=O)Nc3cc(-c4noc(CC5CCCO5)n4)ccc3C)cnc2c1]

To a solution of 553-1 (0.09 g, 1 eq) in NMP (3 mL) was added di(imidazol-1-yl)methanone (123 mg, 1.1 eq). The mixture was stirred at 25° C. for 0.5 hour. Then 553-2 (200 mg, 1.0 eq) was added. The mixture was stirred at 120° C. for 2 hours. The reaction mixture was concentrated under reduced pressure. The residue was purified by prep-HPLC (FA condition; column: Waters Xbridge 150*25 mm*5 um; mobile phase: [water(NH₄HCO₃)-ACN]; gradient:366%-66% B over 9 min) to give 7-methyl-N-(2-methyl-5-(5-((tetrahydrofuran-2-yl)methyl)-1,2,4-oxadiazol-3-yl)phenyl)imidazo[1,2-a]pyridine-3-carboxamide (I-553, 54.88 mg) as a white solid. ¹H NMR (400 MHz, methanol-d₄) δ=9.37 (d, J=7.2 Hz, 1H), 8.41 (s, 1H), 8.10 (d, J=1.6 Hz, 1H), 7.89 (dd, J=1.6, 8.0 Hz, 1H), 7.51 (s, 1H), 7.46 (d, J=8.0 Hz, 1H), 7.03 (dd, J=1.6, 7.2 Hz, 1H), 4.42 (t, J=6.4 Hz, 1H), 3.93-3.85 (m, 1H), 3.80-3.72 (m, 1H), 3.18 (d, J=6.4 Hz, 2H), 2.49 (s, 3H), 2.40 (s, 3H), 2.25-2.11 (m, 1H), 2.02-1.88 (m, 2H), 1.82-1.69 (m, 1H). MS (ESI): m/z for C₂₃H₂₃N₅O₃ [M+H]+ calcd. 418.1 [M+H]+ found: 418.1.

### Example 50—Preparation of (S)—N-(5-(5-(3,3-difluoro-2-hydroxypropyl)-1,2,4-oxadiazol-3-yl)-2-methylphenyl)-7-(thiazol-4-yl)imidazo[1,2-a]pyridine-3-carboxamide (I-554)

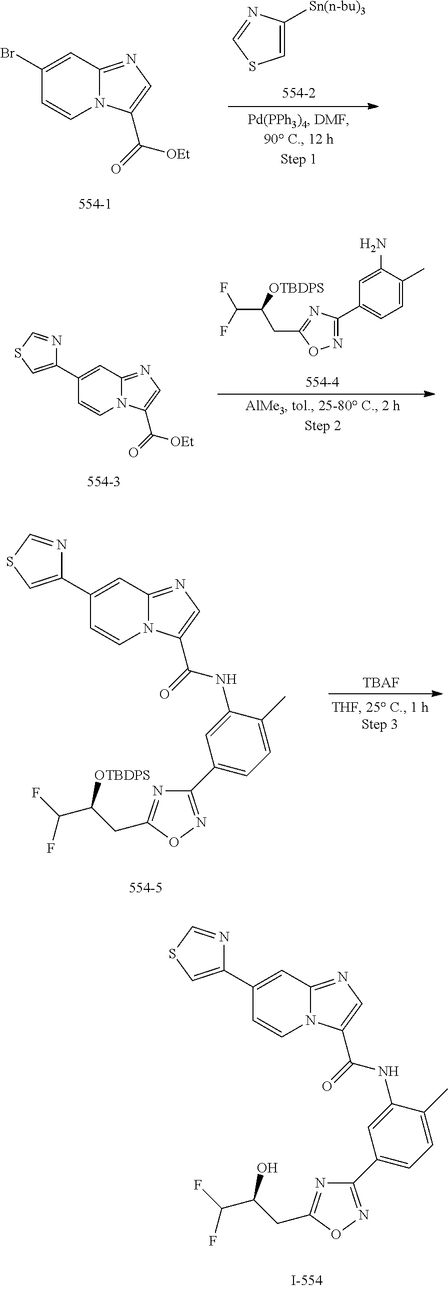

Step 1: To a solution of 554-1 (300 mg, 1.0 eq) in DMF (5 mL) was added 554-2 (459 mg, 1.1 eq) and Pd(PPh₃)₄ (129 mg, 0.1 eq). The mixture was stirred at 90° C. for 12 hours. The reaction mixture was quenched by addition saturated aqueous potassium fluoride solution (40 mL) at 0° C., and then the reaction mixture was diluted with H₂O (40 mL) and extracted with ethyl acetate (100 mL×3). The combined organic layers were washed with brine (50 mL×3), dried over Na₂SO₄, filtered and concentrated under reduced pressure to give a residue. The residue was purified by column chromatography (SiO2, Petroleum ether/ethyl acetate=1/1 to 0.8/1) to give ethyl 7-(thiazol-4-yl)imidazo[1,2-a]pyridine-3-carboxylate (150 mg) as a white solid. MS (ESI): m/z for C₁₃H₁₁N₃O₂S [M+H]⁺ calcd. 274.3, [MH]⁺ found: 274.3.

Step 2: A mixture of 554-4 (185.74 mg, 1.0 eq) and AlMe₃ (2 M, 2.5 eq) in toluene (3 mL) was stirred at 25° C. for 1 hour under N₂, then 554-3 (0.1 g, 1.0 eq) was added into the reaction mixture and the resultant mixture was stirred at 80° C. for 1 hour. The reaction mixture was quenched by addition of a saturated solution of ammonium chloride in water (20 mL) at 0° C., and then diluted with H₂O (20 mL) and extracted with DCM (40 mL×3). The combined organic layers were washed with brine (25 mL×2), dried over Na₂SO₄ filtered and concentrated under reduced pressure to give a residue. The residue was purified by column chromatography (SiO₂, petroleum ether/ethyl acetate=1/1 to 1/2) to give (S)—N-(5-(5-(2-((tert-butyldiphenylsilyl)oxy)-3,3-difluoropropyl)-1,2,4-oxadiazol-3-yl)-2-methylphenyl)-7-(thiazol-4-yl)imidazo[1,2-a]pyridine-3-carboxamide (120 mg) as a white solid. MS (ESI): m/z for C₃₉H₃₆F₂N₆O₃SSi [M+H]⁺ calcd. 735.9 [MH]⁺ found: 735.2.

Step 3: To a solution of (S)—N-(5-(5-(2-((tert-butyldiphenylsilyl)oxy)-3,3-difluoropropyl)-1,2,4-oxadiazol-3-yl)-2-methylphenyl)-7-(thiazol-4-yl)imidazo[1,2-a]pyridine-3-carboxamide (120 mg, 1.0 eq) in THF (2 mL) was added TBAF (1 M, 1.2 eq). The mixture was stirred at 25° C. for 1 hour. The reaction mixture was partitioned between H₂O (15 mL) and ethyl acetate (20 mL×3). The organic phase was separated, washed with brine (5 mL×3), dried over Na₂SO₄, filtered and concentrated under reduced pressure to give a residue. The residue was purified by column chromatography (SiO₂, petroleum ether/ethyl acetate=1/1 to 0/1) to (S)—N-(5-(5-(3,3-difluoro-2-hydroxypropyl)-1,2,4-oxadiazol-3-yl)-2-methylphenyl)-7-(thiazol-4-yl)imidazo[1,2-a]pyridine-3-carboxamide (I-554, 53.59 mg) as a white solid. MS (ESI): m/z for C₂₃H₁₈F₂N₆O₃S [M+H]⁺ calcd. 497.5 [MH]⁺ found: 497.2. ¹H NMR (400 MHz, DMSO-d₆) δ=10.05 (s, 1H), 9.48 (d, J=7.2 Hz, 1H), 9.29 (d, J=2.0 Hz, 1H), 8.63 (s, 1H), 8.53 (d, J=2.0 Hz, 1H), 8.36 (s, 1H), 8.11 (d, J=1.6 Hz, 1H), 7.88-7.76 (m, 2H), 7.51 (d, J=8.0 Hz, 1H), 6.21-5.91 (m, 2H), 4.32-4.18 (m, 1H), 3.28 (d, J=4.0 Hz, 1H), 3.19-3.12 (m, 1H), 2.38 (s, 3H)

### Example 51—Preparation of (S)—N-(5-(5-(3,3-difluoro-2-hydroxypropyl)-1,2,4-oxadiazol-3-yl)-2-methylphenyl)-7-(1-methyl-1H-pyrazol-3-yl)imidazo[1,2-a]pyridine-3-carboxamide (I-555)

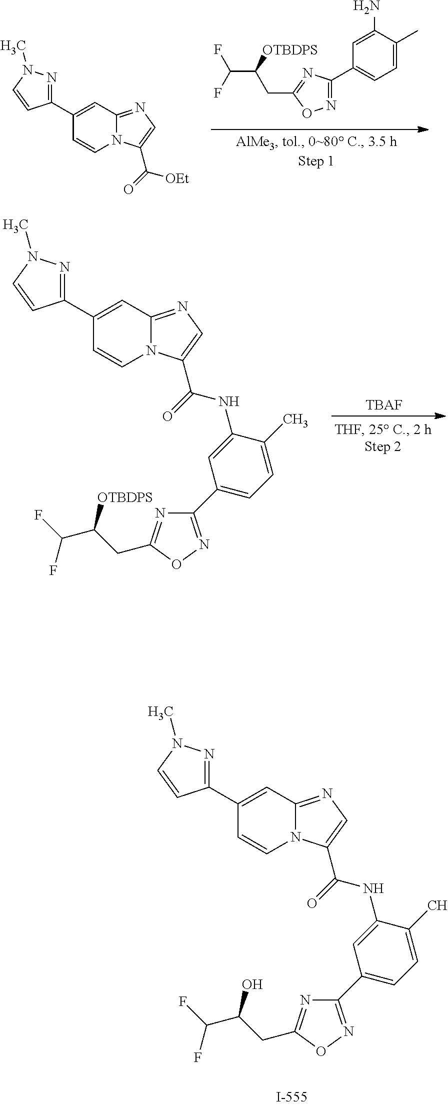

Step 1: To a solution of 5-[5-[(2S)-2-[tert-butyl(diphenyl)silyl]oxy-3,3-difluoro-propyl]-1,2,4-oxadiazol-3-yl]-2-methyl-aniline (188 mg, 1.0 eq) in the toluene (1 mL) was added AlMe₃ (2 M, 2.5 eq) at 0° C. The mixture was stirred at 25° C. for 0.5 hour under an N₂ atmosphere. Then ethyl 7-(1-methylpyrazol-3-yl)imidazo[1,2-a]pyridine-3-carboxylate (100 mg, 1.0 eq) was added and the mixture was stirred at 80° C. for 3 hours under an N₂ atmosphere. The reaction mixture was quenched by the addition of a NH₄Cl solution (1 mL) at 0° C. and filtered, the filtrate was diluted with water (10 mL) and extracted with ethyl acetate (20 mL×3). The combined organic layers were washed with brine (10 mL×3), dried over Na₂SO₄, filtered and concentrated under reduced pressure to give (S)—N-(5-(5-(2-((tert-butyldiphenylsilyl)oxy)-3,3-difluoropropyl)-1,2,4-oxadiazol-3-yl)-2-methylphenyl)-7-(1-methyl-1H-pyrazol-3-yl)imidazo[1,2-a]pyridine-3-carboxamide (210 mg, crude) as a yellow solid. ¹H NMR (400 MHz, DMSO-d₆) δ=10.01 (s, 1H), 9.48-9.43 (m, 1H), 8.61 (s, 1H), 8.12 (s, 1H), 7.99 (d, J=1.6 Hz, 1H), 7.83 (d, J=2.4 Hz, 1H), 7.72-7.58 (m, 4H), 7.52-7.45 (m, 3H), 7.44-7.40 (m, 3H), 7.35-7.30 (m, 3H), 7.00 (d, J=2.4 Hz, 1H), 6.27-5.96 (m, 1H), 4.46-4.34 (m, 1H), 3.93 (s, 3H), 3.33-3.31 (m, 2H), 2.39 (s, 3H), 0.86 (s, 9H). MS (ESI): m/z for C₄₀H₃₉F₂N₇O₃Si [M+H]⁺ calcd. 732.29, [MH]⁺ found: 732.3.

Step 2: To a solution of (S)—N-(5-(5-(2-((tert-butyldiphenylsilyl)oxy)-3,3-difluoropropyl)-1,2,4-oxadiazol-3-yl)-2-methylphenyl)-7-(1-methyl-1H-pyrazol-3-yl)imidazo[1,2-a]pyridine-3-carboxamide (100 mg, 1.0 eq) in THE (1 mL) was added TBAF (1 M, 0.2 mL, 1.46 eq). The mixture was stirred at 25° C. for 2 hours. The reaction was purified directly without work-up. The residue was purified by prep-TLC (SiO₂, ethyl acetate:ethanol=10:1) to give the crude compound which was further purified by prep-HPLC (FA condition; column: C18 150×30 mm; mobile phase: [water(FA)-ACN]; gradient:25%-55% B over 7 min) to give (S)—N-(5-(5-(3,3-difluoro-2-hydroxypropyl)-1,2,4-oxadiazol-3-yl)-2-methylphenyl)-7-(1-methyl-1H-pyrazol-3-yl)imidazo[1,2-a]pyridine-3-carboxamide (I-555, 31.62 mg, 43% yield) as a white solid. ¹H NMR (400 MHz, DMSO-d₆) δ=10.01 (s, 1H), 9.43 (d, J=7.2 Hz, 1H), 8.59 (s, 1H), 8.11 (s, 2H), 7.86-7.78 (m, 2H), 7.68-7.64 (m, 1H), 7.50 (d, J=8.0 Hz, 1H), 6.99 (d, J=2.4 Hz, 1H), 6.22-5.90 (m, 2H), 4.34-4.19 (m, 1H), 3.93 (s, 3H), 3.28 (d, J=4.0 Hz, 1H), 3.20-3.11 (m, 1H), 2.38 (s, 3H). MS (ESI): m/z for C₂₄H₂₁F₂N₇O₃[M+H]⁺ calcd. 494.17, [MH]⁺ found: 494.1.

### Example 52—Preparation of 7-methoxy-N-(2-methyl-5-(5-(5-methylpyridin-2-yl)-1,2,4-oxadiazol-3-yl)phenyl)imidazo[1,2-a]pyridine-3-carboxamide (I-556)

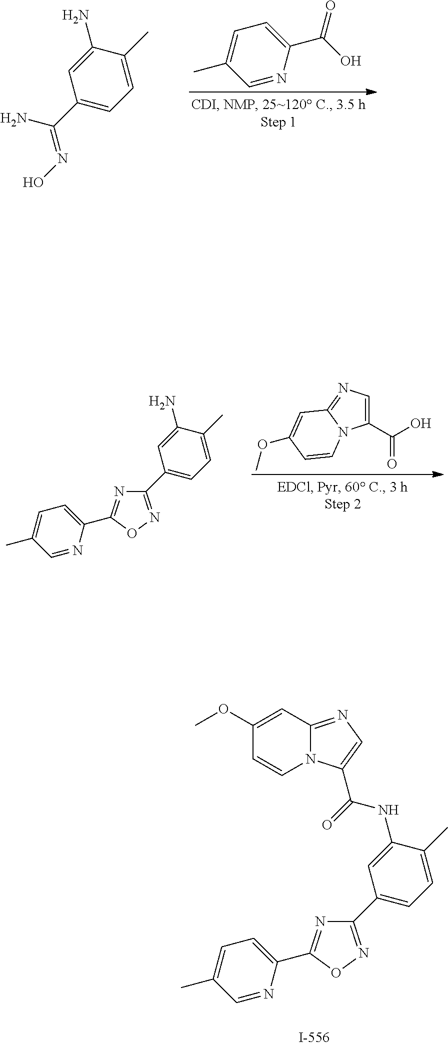

Step 1: To a solution of 5-methylpyridine-2-carboxylic acid (300 mg, 1.0 eq) in NMP (3 mL) was added di(imidazol-1-yl)methanone (426 mg, 1.2 eq). The mixture was stirred at 25° C. for 0.5 hour. The mixture was used without further work up. To the mixture was added 3-amino-N′-hydroxy-4-methyl-benzamidine (353 mg, 1.0 eq). The mixture was stirred at 120° C. for 3 hours. The reaction mixture was diluted with water (20 mL) and extracted with ethyl acetate (30 mL×3). The combined organic layers were washed with brine (20 mL×6), dried over Na₂SO₄, filtered and concentrated under reduced pressure to give 2-methyl-5-(5-(5-methylpyridin-2-yl)-1,2,4-oxadiazol-3-yl) aniline (490 mg, crude) as a yellow solid. ¹H NMR (400 MHz, DMSO-d₆) δ=8.68 (s, 1H), 8.19 (d, J=8.0 Hz, 1H), 7.95-7.89 (m, 1H), 7.39 (d, J=1.6 Hz, 1H), 7.24-7.17 (m, 1H), 7.11 (d, J=7.6 Hz, 1H), 5.21 (s, 2H), 2.43 (s, 3H), 2.13 (s, 3H). MS (ESI): m/z for C₁₅H₁₄N₄O [M+H]⁺ calcd. 267.12, [MH]⁺ found: 267.1.

Step 2: To a solution of 2-methyl-5-(5-(5-methylpyridin-2-yl)-1,2,4-oxadiazol-3-yl) aniline (100 mg, 1.0 eq) in pyridine (1 mL) was added EDCI (115 mg, 1.6 eq) and 7-methoxyimidazo[1,2-a]pyridine-3-carboxylic acid (72 mg, 1.0 eq). The mixture was stirred at 60° C. for 3 hours. The reaction mixture was diluted with water (10 mL) and extracted with ethyl acetate (20 mL×3). The combined organic layers were washed with brine (20 mL×3), dried over Na₂SO₄, filtered and concentrated under reduced pressure to give a residue. The residue was purified by prep-HPLC (FA condition; column: C18 150×30 mm; mobile phase: [water(FA)-ACN]; gradient:25%-55% B over 7 min) to give 7-methoxy-N-(2-methyl-5-(5-(5-methylpyridin-2-yl)-1,2,4-oxadiazol-3-yl)phenyl)imidazo[1,2-a]pyridine-3-carboxamide (I-556, 24.88 mg, 13% yield) as an off-white solid. ¹H NMR (400 MHz, DMSO-d₆) δ=9.93 (s, 1H), 9.28 (d, J=7.6 Hz, 1H), 8.69 (d, J=1.2 Hz, 1H), 8.48 (s, 1H), 8.24 (d, J=8.0 Hz, 1H), 8.18 (d, J=1.6 Hz, 1H), 7.95-7.88 (m, 2H), 7.53 (d, J=8.0 Hz, 1H), 7.17 (d, J=2.4 Hz, 1H), 6.90-6.85 (m, 1H), 3.90 (s, 3H), 2.44 (s, 3H), 2.38 (s, 3H). MS (ESI): m/z for C₂₄H₂₀N₆O₃ [M+H]⁺ calcd. 441.16, [MH]⁺ found. 441.1.

### Example 53—Preparation of N-(2-methyl-5-(5-methyl-1,2,4-oxadiazol-3-yl)phenyl)-6-(thiazol-4-yl)pyrazolo[1,5-a]pyridine-3-carboxamide (I-559)

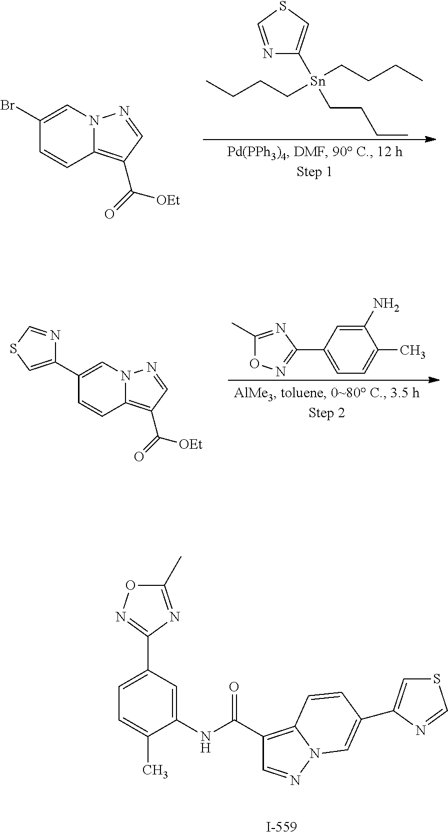

Step 1: To a solution of ethyl 6-bromopyrazolo[1,5-a]pyridine-3-carboxylate (300 mg, 1.0 eq) in DMF (5 mL) was added tributyl(thiazol-4-yl)stannane (459 mg, 1.1 eq) and Pd(PPh₃)₄ (129 mg, 0.1 eq). The mixture was stirred at 90° C. for 12 hours. The reaction mixture was quenched by saturated aqueous potassium fluoride solution (40 mL) at 0° C., and then diluted with H₂O (40 mL) and extracted with ethyl acetate (100 mL×3). The combined organic layers were washed with brine (50 mL×3), dried over Na₂SO₄, filtered and concentrated under reduced pressure to give a residue. The residue was purified by column chromatography (SiO₂, petroleum ether/ethyl acetate=3/1) to give ethyl 6-(thiazol-4-yl)pyrazolo[1,5-a]pyridine-3-carboxylate (170 mg, 48% yield) as a white solid. ¹H NMR (400 MHz, DMSO-d₆) δ=9.38 (s, 1H), 9.28 (d, J=2.0 Hz, 1H), 8.49 (s, 1H), 8.40 (d, J=2.0 Hz, 1H), 8.24-8.20 (m, 1H), 8.15-8.11 (m, 1H), 4.36-4.28 (m, 2H), 1.38-1.32 (m, 3H). MS (ESI): m/z for C₁₃H₁₁N₃O₂S [M+H]⁺ calcd. 274.06, [MH]⁺ found: 274.0.

Step 2: To a solution of 2-methyl-5-(5-methyl-1,2,4-oxadiazol-3-yl) aniline (48 mg, 1.0 eq) in the toluene (1 mL) was added AlMe₃ (2 M, 2.5 eq) at 0° C. The mixture was stirred at 25° C. for 0.5 hour under N₂ atmosphere. Then ethyl 6-(thiazol-4-yl)pyrazolo[1,5-a]pyridine-3-carboxylate (70 mg, 1.0 eq) was added and the mixture was stirred at 80° C. for 3 hours under an N₂ atmosphere. The reaction mixture was quenched by the addition of an NH₄Cl solution (1 mL) at 0° C. and filtered, the filtrate was diluted with water (10 mL) and extracted with ethyl acetate (20 mL×3). The combined organic layers were washed with brine (10 mL×3), dried over Na₂SO₄, filtered and concentrated under reduced pressure to give a residue. The residue was purified by prep-HPLC (FA condition; column: C18 150×30 mm; mobile phase: [water(FA)-ACN]; gradient:45%-75% B over 7 min) to give N-(2-methyl-5-(5-methyl-1,2,4-oxadiazol-3-yl)phenyl)-6-(thiazol-4-yl)pyrazolo[1,5-a]pyridin-3-carboxamide (I-559, 10.45 mg, 9% yield) as a white solid. ¹H NMR (400 MHz, DMSO-d₆) δ=9.78 (s, 1H), 9.38 (s, 1H), 9.29 (d, J=2.0 Hz, 1H), 8.83 (s, 1H), 8.40 (d, J=1.6 Hz, 1H), 8.33-8.29 (m, 1H), 8.18-8.14 (m, 1H), 8.12 (d, J=1.6 Hz, 1H), 7.80-7.86 (m, 1H), 7.47 (d, J=8.0 Hz, 1H), 2.67 (s, 3H), 2.37 (s, 3H). MS (ESI): m/z for C₂₁H₁₆N₆O₂S [M+H]⁺ calcd. 417.11, [MH]⁺ found. 417.1.

### Example 54—Preparation of (S)—N-(5-(5-(3,3-difluoro-2-hydroxypropyl)-1,2,4-oxadiazol-3-yl)-2-methylphenyl)-6-(thiazol-4-yl)imidazo[1,2-a]pyridine-3-carboxamide (I-560)

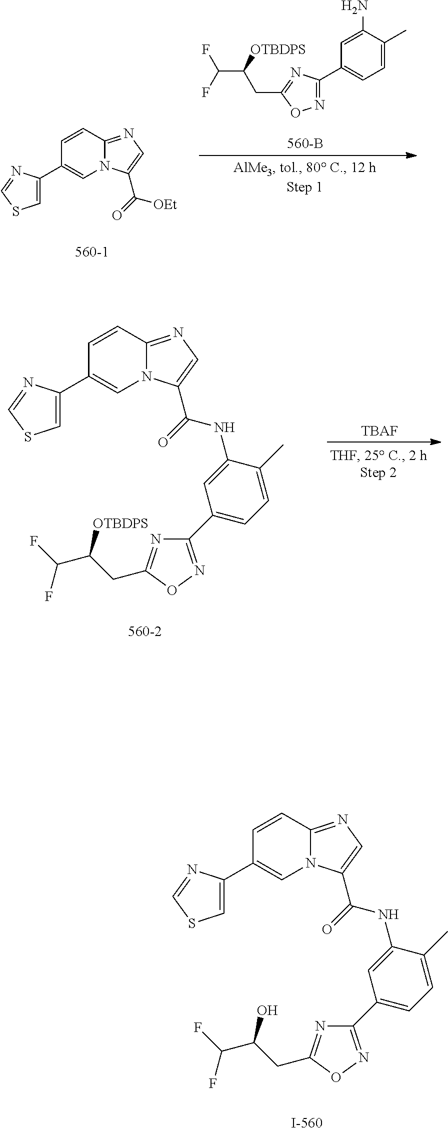

Step 1: To a solution of 560-B (186 mg, 1.0 eq) in toluene (2 mL) was added AlMe₃ (2 M, 2.5 eq) and the reaction mixture was stirred at 25° C. for 0.5 hour. Then 560-1 (100 mg, 1 eq) was added and the reaction mixture was stirred at 80° C. for 2 hours. The reaction mixture was quenched with NH₄Cl (20 mL) then was extracted with ethyl acetate (50 mL×3). The combined organic layers were washed with water (25 mL×2), dried over Na₂SO₄, filtered and concentrated under reduced pressure to give a residue. The residue was purified by column chromatography (SiO₂, petroleum ether/ethyl acetate=5/1 to 0/1) to give (S)—N-(5-(5-(2-((tert-butyldiphenylsilyl)oxy)-3,3-difluoropropyl)-1,2,4-oxadiazol-3-yl)-2-methylphenyl)-6-(thiazol-4-yl)imidazo[1,2-a]pyridine-3-carboxamide (100 mg) as a white solid. MS (ESI): m/z for C₃₉H₃₆F₂N₆O₃SSi [M+H]+ calcd. 735.4 [M+H]+ found: 735.4.

Step 2: To a solution of (S)—N-(5-(5-(2-((tert-butyldiphenylsilyl)oxy)-3,3-difluoropropyl)-1,2,4-oxadiazol-3-yl)-2-methylphenyl)-6-(thiazol-4-yl)imidazo[1,2-a]pyridine-3-carboxamide (100 mg, 1.0 eq) in THE (2 mL) was added TBAF (1 M, 1.1 eq). The mixture was stirred at 25° C. for 2 hours. The reaction mixture was diluted with H₂O (20 mL) and extracted with ethyl acetate (50 mL×2). The combined organic layers were washed with brine (15 mL×2), dried over Na₂SO₄, filtered and concentrated under reduced pressure to give a residue. The residue was purified by prep-HPLC (FA condition; column: C18 150×30 mm; mobile phase: [water(FA)-ACN]; gradient:355%-65% B over 7 min) to give (S)—N-(5-(5-(3,3-difluoro-2-hydroxypropyl)-1,2,4-oxadiazol-3-yl)-2-methylphenyl)-6-(thiazol-4-yl)imidazo[1,2-a]pyridine-3-carboxamide (I-560, 9.85 mg) as a white solid. ¹H NMR (400 MHz, methanol-d₄) δ=10.18 (s, 1H), 9.09 (d, J=2.0 Hz, 1H), 8.50 (s, 1H), 8.17 (d, J=1.6 Hz, 1H), 8.14 (dd, J=1.6, 9.2 Hz, 1H), 8.05 (d, J=2.0 Hz, 1H), 7.92 (dd, J=1.6, 8.0 Hz, 1H), 7.85-7.78 (m, 1H), 7.48 (d, J=8.0 Hz, 1H), 6.10-5.75 (m, 1H), 4.46-4.28 (m, 1H), 3.27-3.17 (m, 2H), 2.44 (s, 3H). MS (ESI): m/z for C₂₃H₁₈F₂N₆O₃S [M+H]⁺ calcd. 497.1 [M+H]⁺ found. 497.1

### Example 55—Preparation of N-(5-(5-(3-hydroxy-3-methylbutyl)-1,2,4-oxadiazol-3-yl)-2-methylphenyl)-7-methoxyimidazo[1,2-a]pyridine-3-carboxamide (I-561)

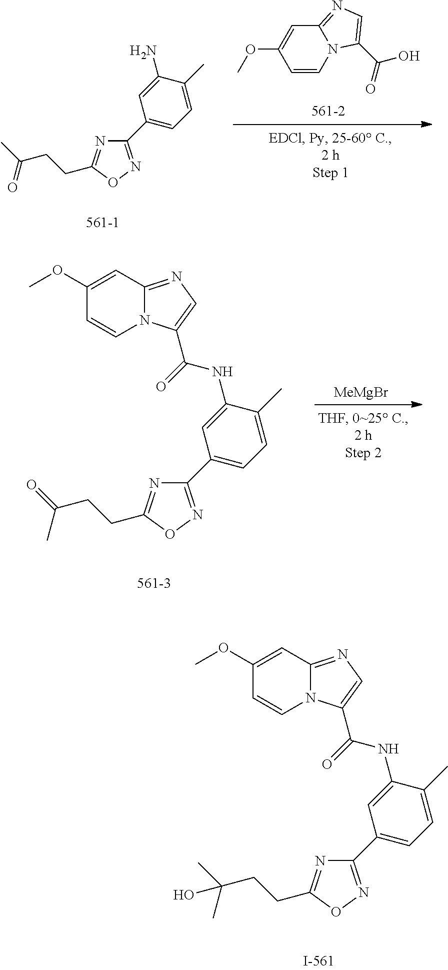

Step 1: To a solution of 561-1 (235 mg, 1.0 eq) in pyridine (6 mL) was added EDCI (469 mg, 2.0 eq). The mixture was stirred at 25° C. for 0.5 hour. Then 561-2 was added and the mixture was stirred at 60° C. for 1.5 hours. The reaction mixture was concentrated under reduced pressure to remove solvent. The residue was purified by column chromatography (SiO₂, petroleum ether/ethyl acetate=1/1 to 0/1) to give 7-methoxy-N-(2-methyl-5-(5-(3-oxobutyl)-1,2,4-oxadiazol-3-yl)phenyl)imidazo[1,2-a]pyridine-3-carboxamide (200 mg) as a white solid. ¹H NMR (400 MHz, DMSO-d₆) δ=9.89 (s, 1H), 9.26 (d, J=7.6 Hz, 1H), 8.45 (s, 1H), 8.03 (d, J=1.6 Hz, 1H), 7.78 (dd, J=1.6, 8.0 Hz, 1H), 7.47 (d, J=8.0 Hz, 1H), 7.16 (d, J=2.4 Hz, 1H), 6.87 (dd, J=2.4, 7.6 Hz, 1H), 3.90 (s, 3H), 3.16-3.05 (m, 4H), 2.35 (s, 3H), 2.17 (s, 3H)

Step 2: To a solution of 7-methoxy-N-(2-methyl-5-(5-(3-oxobutyl)-1,2,4-oxadiazol-3-yl)phenyl)imidazo[1,2-a]pyridine-3-carboxamide (150 mg, 1.0 eq) in THE (3 mL) was added MeMgBr (3 M, 10 eq) at 0° C. The mixture was stirred at 25° C. for 2 hours under an N₂ atmosphere. The reaction mixture was quenched by addition of a saturated solution of ammonium chloride in water (10 mL) at 0° C., and then diluted with H₂O (10 mL) and extracted with ethyl acetate (20 mL×3). The combined organic layers were dried over Na₂SO₄, filtered and concentrated under reduced pressure to give a residue. The residue was purified by prep-HPLC (column: C18 150×30 mm; mobile phase: [water(FA)-ACN]; gradient:20%-500% B over 7 min) to give N-(5-(5-(3-hydroxy-3-methylbutyl)-1,2,4-oxadiazol-3-yl)-2-methylphenyl)-7-methoxyimidazo[1,2-a]pyridine-3-carboxamide (I-561, 35.04 mg) as a white solid. ¹H NMR (400 MHz, DMSO-d₆) δ=9.89 (s, 1H), 9.27 (d, J=7.6 Hz, 1H), 8.45 (s, 1H), 8.05 (d, J=1.2 Hz, 1H), 7.79 (dd, J=1.6, 8.0 Hz, 1H), 7.47 (d, J=8.0 Hz, 1H), 7.16 (d, J=2.4 Hz, 1H), 6.87 (dd, J=2.4, 7.6 Hz, 1H), 4.41 (s, 1H), 3.90 (s, 3H), 3.05-2.99 (m, 2H), 2.35 (s, 3H), 1.94-1.85 (m, 2H), 1.14 (s, 6H). MS (ESI): m/z for C₂₃H₂₅N₅O₄ [M+H]⁺ calcd. 436.5 [MH]⁺ found: 436.2.

### Example 56—Preparation of (S)—N-(5-(5-(3,3-difluoro-2-hydroxypropyl)-1,2,4-oxadiazol-3-yl)-2-methylphenyl)-6-(1-methyl-1H-pyrazol-3-yl)imidazo[1,2-a]pyridine-3-carboxamide (I-562)

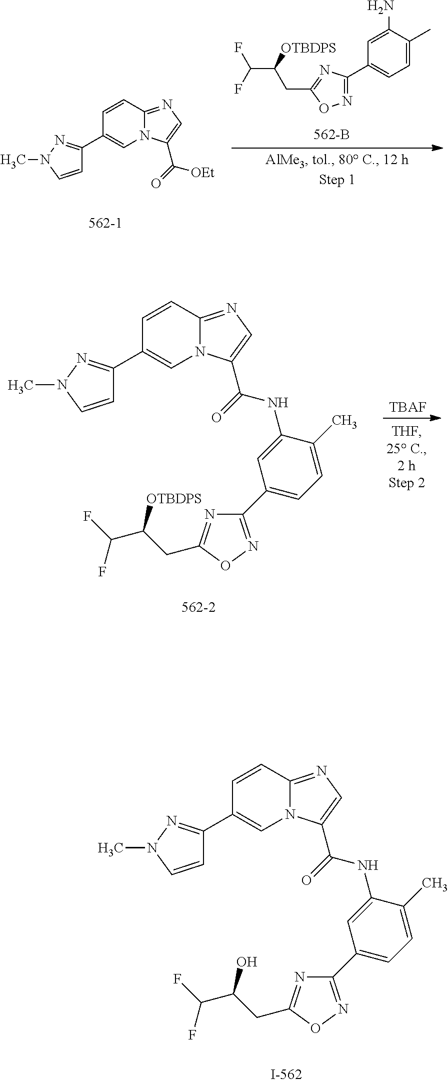

Step 1: To a solution of 562-B (310 mg, 1.1 eq) in toluene (3 mL) was added AlMe₃ (2 M, 2.5 eq) and the reaction mixture was stirred at 25° C. for 0.5 hour. Then 562-1 (150 mg, 1.0 eq) was added and the reaction mixture was stirred at 80° C. for 2 hours. The reaction mixture was quenched with aqueous NH₄Cl (20 mL) and then was extracted with ethyl acetate (50 mL×3). The combined organic layers were washed with water (25 mL×2), dried over Na₂SO₄, filtered and concentrated under reduced pressure to give a residue. The residue was purified by column chromatography (SiO₂, petroleum ether/ethyl acetate=5/1 to 0/1) to give (S)—N-(5-(5-(2-((tert-butyldiphenylsilyl)oxy)-3,3-difluoropropyl)-1,2,4-oxadiazol-3-yl)-2-methylphenyl)-6-(1-methyl-1H-pyrazol-3-yl)imidazo[1,2-a]pyridine-3-carboxamide (0.3 g) as a white solid. MS (ESI): m/z for C₄₀H₃₉F₂N₇O₃Si [M+H]+ calcd. 732.3 [M+H]+ found. 732.3

Step 2: To a solution of (S)—N-(5-(5-(2-((tert-butyldiphenylsilyl)oxy)-3,3-difluoropropyl)-1,2,4-oxadiazol-3-yl)-2-methylphenyl)-6-(1-methyl-1H-pyrazol-3-yl)imidazo[1,2-a]pyridine-3-carboxamide (150 mg, 1.0 eq) in THE (2 mL) was added TBAF (1 M, 1.1 eq). The mixture was stirred at 25° C. for 1 hour. The reaction mixture was diluted with H₂O (30 mL) and was extracted with ethyl acetate (50 mL×2). The combined organic layers were washed with water (25 mL×2), dried over Na₂SO₄, filtered and concentrated under reduced pressure to give a residue. The residue was purified by column chromatography (SiO₂, petroleum ether/ethyl acetate=5/1 to 0/1) to give (S)—N-(5-(5-(3,3-difluoro-2-hydroxypropyl)-1,2,4-oxadiazol-3-yl)-2-methylphenyl)-6-(1-methyl-1H-pyrazol-3-yl)imidazo[1,2-a]pyridine-3-carboxamide (I-562, 59.83 mg) as an off-white solid. MS (ESI): m/z for C₂₄H₂₁F₂N₇O₃[M+H]+ calcd. 494.2 [M+H]+ found: 494.2. ¹H NMR (400 MHz, methanol-d₄) δ=9.91 (s, 1H), 8.48 (s, 1H), 8.17 (s, 1H), 8.06-7.88 (m, 2H), 7.77 (d, J=9.2 Hz, 1H), 7.66 (d, J=2.0 Hz, 1H), 7.48 (d, J=8.0 Hz, 1H), 6.70 (d, J=2.0 Hz, 1H), 6.10-5.75 (m, 1H), 4.44-4.25 (m, 1H), 3.96 (s, 3H), 3.29-3.20 (m, 2H), 2.44 (s, 3H)

### Example 57—Preparation of (S)—N-(5-(5-(3,3-difluoro-2-hydroxypropyl)-1,2,4-oxadiazol-3-yl)-2-methylphenyl)-7-ethoxyimidazo[1,2-a]pyridine-3-carboxamide (I-566)

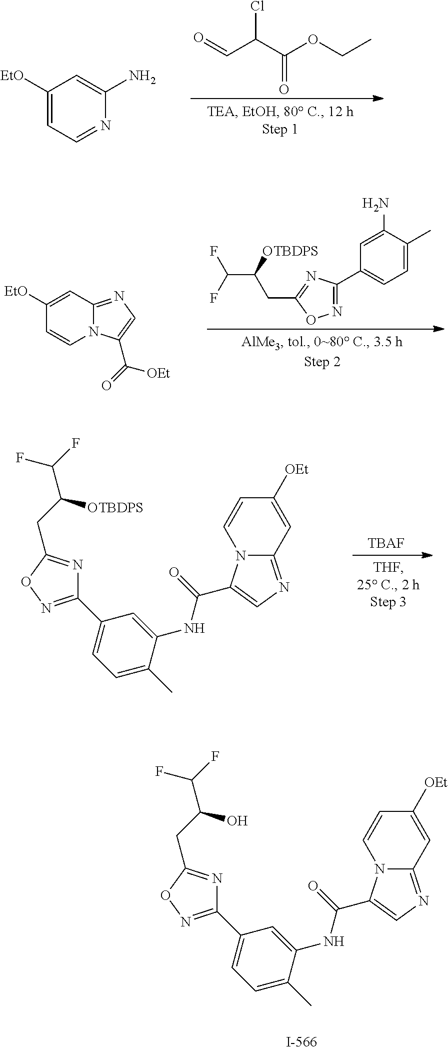

Step 1: To a solution of 4-ethoxypyridin-2-amine (500 mg, 1.0 eq) in EtOH (5 mL) was added triethylamine (1.10 g, 1.51 mL, 3 eq) and ethyl 2-chloro-3-oxo-propanoate (1.09 g, 2.0 eq). The mixture was stirred at 80° C. for 12 hours. The reaction mixture was diluted with water (20 mL) and extracted with ethyl acetate (30 mL×3). The combined organic layers were washed with brine (30 mL×3), dried over Na₂SO₄, filtered and concentrated under reduced pressure to give ethyl 7-ethoxyimidazo[1,2-a]pyridine-3-carboxylate (900 mg, crude) as a yellow solid. ¹H NMR (400 MHz, DMSO-d₆) δ=9.01 (d, J=7.6 Hz, 1H), 8.14 (s, 1H), 7.17 (d, J=2.4 Hz, 1H), 6.94-6.89 (m, 1H), 4.36-4.29 (m, 1H), 4.19-4.12 (m, 1H), 1.42-1.28 (m, 6H). MS (ESI): m/z for C₁₂H₁₄N₂O₃ [M+H]⁺ calcd. 235.10, [MH]⁺ found: 235.0.

Step 2: To a solution of (S)-5-(5-(2-((tert-butyldiphenylsilyl)oxy)-3,3-difluoropropyl)-1,2,4-oxadiazol-3-yl)-2-methylaniline (152 mg, 1.0 eq) in the toluene (1 mL) was added AlMe₃ (2 M, 373 μL, 2.5 eq) at 0° C. The mixture was stirred at 25° C. for 0.5 hour under an N₂ atmosphere. Then ethyl 7-ethoxyimidazo[1,2-a]pyridine-3-carboxylate (70 mg, 1.0 eq) was added and the mixture was stirred at 80° C. for 3 hours under an N₂ atmosphere. The reaction mixture was quenched by the addition of an NH₄Cl solution (1 mL) at 0° C. and the reaction mixture was filtered, the filtrate was diluted with water (10 mL) and extracted with ethyl acetate (20 mL×3). The combined organic layers were washed with brine (10 mL×3), dried over Na₂SO₄, filtered and concentrated under reduced pressure to give a residue. The residue was purified by column chromatography (SiO₂, petroleum ether/ethyl acetate=1/1) to give (S)—N-(5-(5-(2-((tert-butyldiphenylsilyl)oxy)-3,3-difluoropropyl)-1,2,4-oxadiazol-3-yl)-2-methylphenyl)-7-ethoxyimidazo[1,2-a]pyridine-3-carboxamide (90 mg, 43% yield) as a white solid. MS (ESI): m/z for C₃₈H₃₉F₂N₅O₄Si [M+H]⁺ calcd. 696.27, [MH]⁺ found. 696.3.

Step 3: To a solution of (S)—N-(5-(5-(2-((tert-butyldiphenylsilyl)oxy)-3,3-difluoropropyl)-1,2,4-oxadiazol-3-yl)-2-methylphenyl)-7-ethoxyimidazo[1,2-a]pyridine-3-carboxamide (90 mg, 1.0 eq) in THE (1 mL) was added TBAF (1 M, 0.2 mL, 1.55 eq). The mixture was stirred at 25° C. for 2 hours. The reaction was purified directly without work-up. The residue was purified by prep-TLC (SiO₂, petroleum ether:ethyl acetate=0:1) to give a crude product which was further purified by prep-HPLC (FA condition; column: C18 150×30 mm; mobile phase: [water(FA)-I]; gradient:22%-52% B over 7 min) to give (S)—N-(5-(5-(3,3-difluoro-2-hydroxypropyl)-1,2,4-oxadiazol-3-yl)-2-methylphenyl)-7-ethoxyimidazo[1,2-a]pyridine-3-carboxamide (I-566, 20.26 mg, 30% yield) as a white solid. ¹H NMR (400 MHz, DMSO-d₆) δ=9.88 (s, 1H), 9.26 (d, J=7.6 Hz, 1H), 8.45 (s, 1H), 8.07 (d, J=1.2 Hz, 1H), 7.-4-7.78 (m, 1H), 7.49 (d, J=8.0 Hz, 1H), 7.13 (d, J=2.4 Hz, 1H), 6.-8-6.82 (m, 1H), 6.-2-5.89 (m, 2H), 4.-1-4.12 (m, 3H), 3.27 (d, J=4.0 Hz, 1H), 3.-9-3.10 (m, 1H), 2.36 (s, 3H), 1.38 (t, J=6.8 Hz, 3H). MS (ESI): m/z for C₂₂H₂₁F₂N₅O₄[M+H]⁺ calcd. 458.16, [MH]⁺ found: 458.1.

### Example 58—Preparation of (S)—N-(5-(5-(3,3-difluoro-2-hydroxypropyl)-1,2,4-oxadiazol-3-yl)-2-methylphenyl)-7-(4-hydroxybutoxy)imidazo[1,2-a]pyridine-3-carboxamide (I-568)

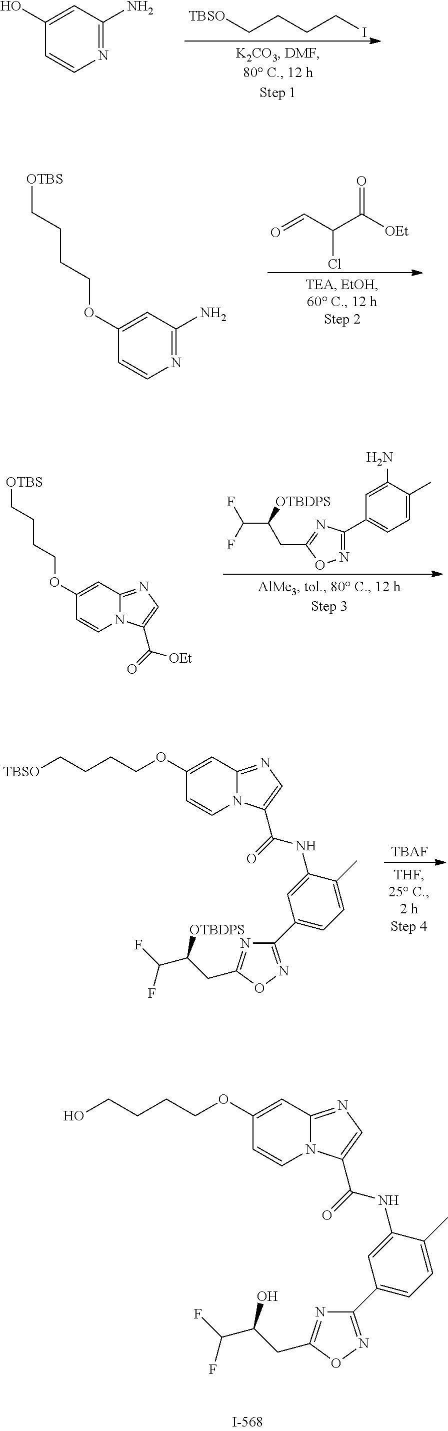

Step 1: To a solution of 2-aminopyridin-4-ol (500 mg, 1.0 eq) in DMF (10 mL) was added K₂CO₃ (1.26 g, 2.0 eq) and tert-butyl-(4-iodobutoxy)-dimethyl-silane (1.43 g, 1.18 mL, 1.0 eq). The mixture was stirred at 80° C. for 12 hours. The mixture was added 20 mL of water and extracted with ethyl acetate (20 mL×3). The combined organic layers were washed with brine 20 mL, dried over Na₂SO₄, filtered and concentrated under reduced pressure to give a residue. The residue was purified by column chromatography (SiO₂, petroleum ether/ethyl acetate=1/9) to give 4-(4-((tert-butyldimethylsilyl)oxy)buto529midazoldin-2-amine (300 mg, 20.28% yield, 91% purity) as white solid. MS (ESI): m/z for: C₁₅H₂₈N₂O₂Si [M+H]⁺ calcd. 297.19 [M+H]⁺ found: 297.2

Step 2: To a solution of 4-(4-((tert-butyldimethylsilyl)oxy)buto529midazoldin-2-amine (300 mg, 1.0 eq) and ethyl 2-chloro-3-oxo-propanoate (152 mg, 1.0 eq) in EtOH (6 mL) was added triethylamine (205 mg, 2.0 eq). The mixture was stirred at 80° C. for 12 hours. The mixture was concentrated to give a residue. The residue was purified by column chromatography (SiO₂, petroleum ether/ethyl acetate=5/1) to give ethyl 7-(4-((tert-butyldimethylsilyl)oxy)butoxy)imidazo[1,2-a]pyridine-3-carboxylate (300 mg, 68% yield) as yellow solid. MS (ESI): m/z for: C₂₀H₃₂N₂O₄Si [M+H]⁺ calcd. 393.21 [M+H]⁺ found: 393.2. ¹H NMR (400 MHz, chloroform-d) δ=9.02 (d, J=7.6 Hz, 1H), 8.11 (s, 1H), 6.90 (d, J=2.4 Hz, 1H), 6.-8-6.61 (m, 1H), 4.-8-4.29 (m, 2H), 4.01 (t, J=6.4 Hz, 2H), 3.63 (t, J=6.4 Hz, 2H), 1.-0-1.80 (m, 2H), 1.-0-1.56 (m, 4H), 1.34 (t, J=7.2 Hz, 3H), 0.84 (s, 9H), 0.05-−0.06 (m, 6H).

Step 3: To a solution (S)-5-(5-(2-((tert-butyldiphenylsilyl)oxy)-3,3-difluoropropyl)-1,2,4-oxadiazol-3-yl)-2-methylaniline (230 mg, 1.0 eq) in toluene (2 mL) was added AlMe₃ (2 M, 2.5 eq). The mixture was stirred at 25° C. for 0.5 hours. To the mixture was added ethyl 7-(4-((tert-butyldimethylsilyl)oxy)butoxy)imidazo[1,2-a]pyridine-3-carboxylate (198 mg 1.0 eq). The mixture was stirred at 80° C. for 2 hours, and then the mixture was cooled it to rt. The mixture was poured into 10 mL of water and extracted with DCM (20 mL*3). The combined organic layers were washed with brine (20 mL), dried over Na₂SO₄, filtered and concentrated under reduced pressure to give a residue. The residue was purified by column chromatography (SiO2, petroleum ether/ethyl acetate=5/1 to 3/1) to give (S)-7-(4-((tert-butyldimethylsilyl)oxy)butoxy)-N-(5-(5-(2-((tert-butyldiphenylsilyl)oxy)-3,3-difluoropropyl)-1,2,4-oxadiazol-3-yl)-2-methylphenyl)imidazo[1,2-a]pyridine-3-carboxamide (250 mg, 63% yield) as yellow oil. MS (ESI): m/z for: C₄₆H₅₇F₂N₅O₅Si₂ [M+H]⁺ calcd. 854.39 [M+H]⁺ found: 854.4.

Step 4: To a solution of (S)-7-(4-((tert-butyldimethylsilyl)oxy)butoxy)-N-(5-(5-(2-((tert-butyldiphenylsilyl)oxy)-3,3-difluoropropyl)-1,2,4-oxadiazol-3-yl)-2-methylphenyl)imidazo[1,2-a]pyridine-3-carboxamide (250 mg, 1.0 eq) in THE (5 mL) was added TBAF (1 M, 2.0 eq). The mixture was stirred at 25° C. for 2 hours. The mixture was concentrated to give a residue. The residue was purified by column chromatography (SiO₂, ethyl acetate/EtOH=1:0 to 10:1) to give a white solid, which was further purified by prep-HPLC (column: C18 150×30 mm; mobile phase: [water(FIACN]; gradient:18%-4% B over 8 min) to give (S)—N-(5-(5-(3,3-difluoro-2-hydroxypropyl)-1,2,4-oxadiazol-3-yl)-2-methylphenyl)-7-(4-hydroxybutoxy)imidazo[1,2-a]pyridine-3-carboxamide (I-568, 59.8 mg, 37.32% yield) as white solid. MS (ESI): m/z for: C₂₄H₂₅F₂N₅O₅[M+H]⁺ calcd. 502.18 [M+H]⁺ found: 502.2. ¹H NMR (400 MHz, DMSO-d₆) δ=9.88 (s, 1H), 9.25 (d, J=7.6 Hz, 1H), 8.44 (s, 1H), 8.07 (d, J=1.6 Hz, 1H), −7.88-7.77 (m, 1H), 7.48 (d, J=8.0 Hz, 1H), 7.13 (d, J=2.4 Hz, 1H), −6.90-6.80 (m, 1H), −6.22-5.87 (m, 2H), 4.50 (s, 1H), 4.25 (d, J=4.4 Hz, 1H), 4.11 (t, J=6.4 Hz, 2H), 3.47 (t, J=6.4 Hz, 2H), −3.31-3.25 (m, 1H), −3.19-3.10 (m, 1H), 2.35 (s, 3H), −1.85-1.74 (m, 2H), −1.66-1.53 (m, 2H).

### Example 59—Preparation of (S)—N-(5-(5-(3,3-difluoro-2-hydroxypropyl)-1,2,4-oxadiazol-3-yl)-2-methylphenyl)-7-(2-hydroxyethoxy)imidazo[1,2-a]pyridine-3-carboxamide (I-570)

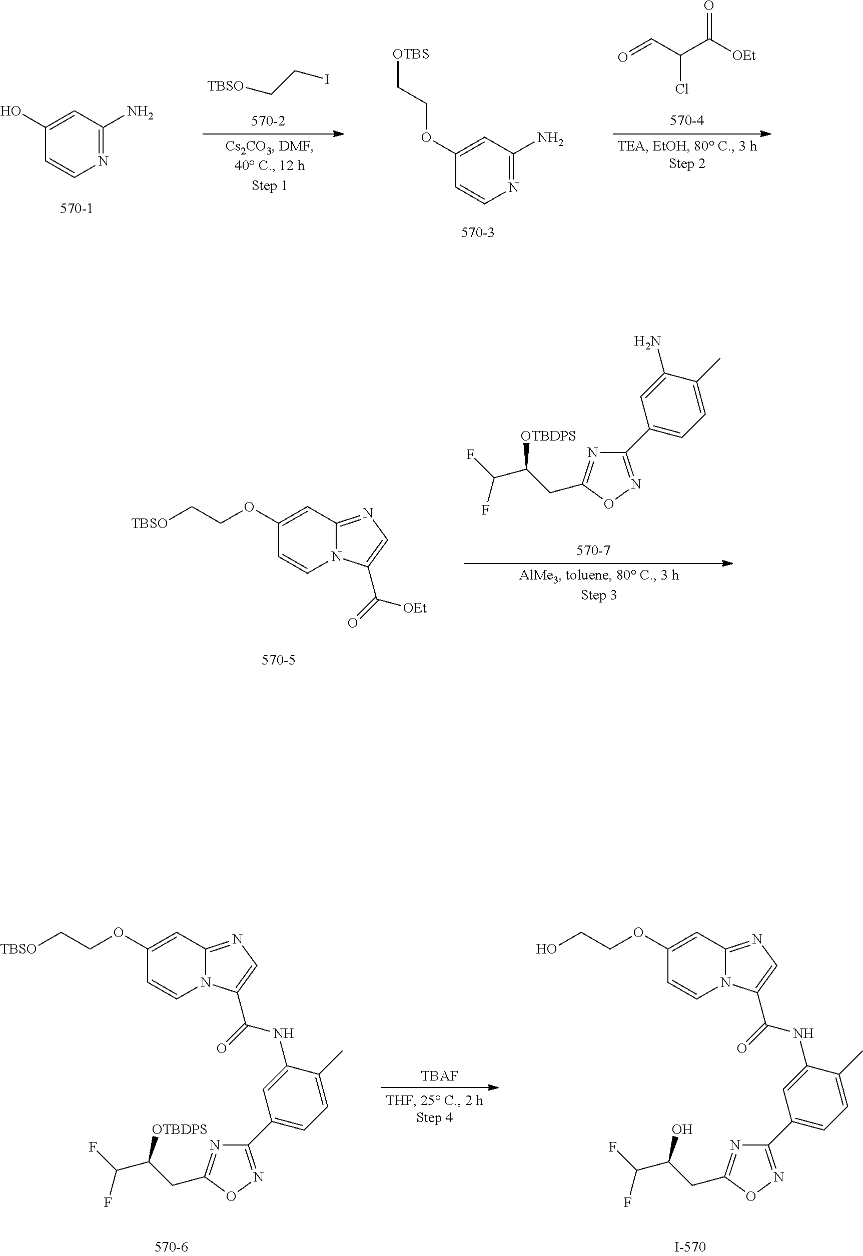

Step 1: To a solution of 570-1 (2.0 g, 1.0 eq) in DMF (20 mL) was added Cs₂CO₃ (8.88 g, 1.5 eq) and 570-2 (10.40 g, 2.0 eq). The mixture was stirred at 40° C. for 12 hours. The reaction mixture was diluted with H₂O (50 mL) and extracted with ethyl acetate (50 mL×4). The combined organic layers were washed with brine (50 mL×6), dried over Na₂SO₄, filtered and concentrated under reduced pressure to give a residue. The residue was purified by column chromatography (SiO₂, ethyl acetate: MeOH=100/1 to 10/1) to-give -(2-((tert-butyldimethylsilyl)oxy)e531midazolyridin-2-amine (2.57 g) as a white solid. MS (ESI): m/z for C₁₃H₂₄N₂O₂Si [M+H]+ calcd. 269.1 [M+H]+ found: 269.1

Step 2: To a solution of 4-(2-((tert-butyldimethylsilyl)oxy)e531midazolyridin-2-amine (600 mg, 1.0 eq) in EtOH (10 mL) was added triethylamine (678.55 mg, 3.0 eq) and 570-4 (673 mg, 2.0 eq). The mixture was stirred at 80° C. for 3 hours. The reaction mixture was diluted with H₂O (30 mL) and extracted with ethyl acetate (40 mL×3). The combined organic layers were washed with brine (20 mL×3), dried over Na₂SO₄, filtered and concentrated under reduced pressure to give a residue. The residue was purified by column chromatography (SiO₂, petroleum ether/ethyl acetate=5/1 to 1/1) to give ethyl 7-(2-((tert-butyldimethylsilyl)oxy)ethoxy)imidazo[1,2-a]pyridine-3-carboxylate (450 mg) as a white solid. MS (ESI): m/z for C₁₈H₂₈N₂O₄Si [M+H]+ calcd. 365.1 [M+H]+ found. 365.1

Step 3: To a solution of 570-7 (306.39 mg, 1.1 eq) in toluene (4 mL) was added AlMe₃ (2 M, 2.5 eq), the reaction mixture was stirred at 25° C. for 0.5 hours, then ethyl 7-(2-((tert-butyldimethylsilyl)oxy)ethoxy)imidazo[1,2-a]pyridine-3-carboxylate (200 mg, 1.0 eq) was added, and the reaction mixture was stirred at 80° C. for 12 hours. The reaction mixture was quenched with NH₄Cl (50 mL) and then was extracted with ethyl acetate (50 mL×3). The combined organic layers were washed with water (25 mL×2), dried over Na₂SO₄, filtered and concentrated under reduced pressure to give a residue. The residue was purified by column chromatography (SiO₂, petroleum ether/ethyl acetate=5/1 to 0/1) to give (S)-7-(2-((tert-butyldimethylsilyl)oxy)ethoxy)-N-(5-(5-(3,3-difluoro-2-hydroxypropyl)-1,2,4-oxadiazol-3-yl)-2-methylphenyl)imidazo[1,2-a]pyridine-3-carboxamide (200 mg) as a white solid. MS (ESI): m/z for C₂₈H₃₅F₂N₅O5Si [M+H]+ calcd. 826.4 [M+H]+ found. 826.4

Step 4: To a solution of (S)-7-(2-((tert-butyldimethylsilyl)oxy)ethoxy)-N-(5-(5-(3,3-difluoro-2-hydroxypropyl)-1,2,4-oxadiazol-3-yl)-2-methylphenyl)imidazo[1,2-a]pyridine-3-carboxamide (120 mg, 1.0 eq) in THE (2 mL) was added TBAF (1 M, 2.0 eq). The mixture was stirred at 25° C. for 2 hours. The reaction mixture was diluted with H₂O (30 mL) and extracted with ethyl acetate (25 mL×4). The combined organic layers were washed with brine (15 mL×2), dried over Na₂SO₄, filtered and concentrated under reduced pressure to give a residue. The residue was purified by column chromatography (SiO₂, petroleum ether/ethyl acetate=10/1 to 0/1) to give (S)—N-(5-(5-(3,3-difluoro-2-hydroxypropyl)-1,2,4-oxadiazol-3-yl)-2-methylphenyl)-7-(2-hydroxyethoxy)imidazo[1,2-a]pyridine-3-carboxamide (I-570, 30.48 mg) as a white solid. ¹H NMR (400 MHz, DMSO-d₆) δ=9.89 (s, 1H), 9.27 (d, J=7.2 Hz, 1H), 8.45 (s, 1H), 8.07 (d, J=1.6 Hz, 1H), 7.81 (dd, J=1.6, 8.0 Hz, 1H), 7.49 (d, J=8.0 Hz, 1H), 7.15 (d, J=2.4 Hz, 1H), 6.87 (dd, J=2.4, 7.6 Hz, 1H), −6.23-5.89 (m, 2H), 4.97 (t, J=5.6 Hz, 1H), −4.33-4.18 (m, 1H), 4.13 (t, J=4.8 Hz, 2H), −3.81-3.73 (m, 2H), 3.27 (d, J=3.6 Hz, 1H), 3.16 (d, J=9.2 Hz, 1H), 2.35 (s, 3H). MS (ESI): m/z for C₂₂H₂₁F₂N₅O₅[M+H]+ calcd. 474.2 [M+H]+ found: 474.2.

### Example 60—Preparation of N-(5-(5-((S)-3,3-difluoro-2-hydroxypropyl)-1,2,4-oxadiazol-3-yl)-2-methylphenyl)-7-(2-hydroxypropoxy)imidazo[1,2-a]pyridine-3-carboxamide (I-571)

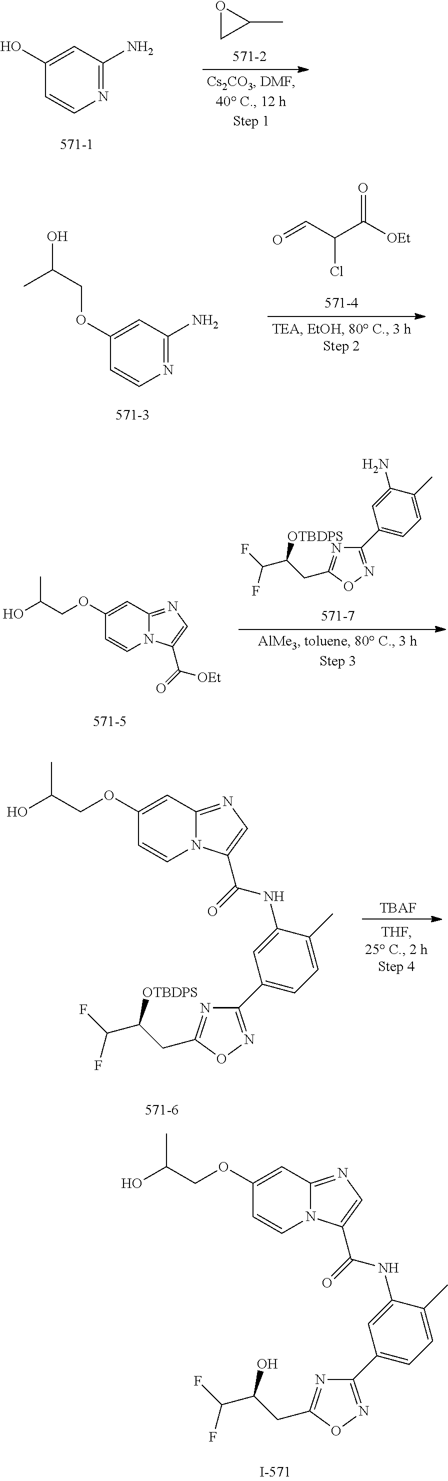

Step 1: To a solution of 571-1 (2.0 g, 1.0 eq) in DMF (20 mL) was added Cs₂CO₃ (8.88 g, 1.5 eq) and 571-2 (2.11 g, 2.0 eq). The mixture was stirred at 40° C. for 12 hours. The reaction mixture was diluted with H₂O (200 mL) and lyophilized. The residue was dissolved in DCM: MeOH (10:1; 100 mL), and the mixture was stirred at 25° C. for 0.5 hour. The suspension was filtered, and the filtrate was concentrated under reduced pressure to give 1-((2-aminopyridin-4-yl)oxy)propan-2-ol (1.6 g, crude) as a white solid. MS (ESI): m/z for C₈H₁₂N₂O₂ [M+H]+ calcd. 169.2 [M+H]+ found: 169.2.

Step 2: To a solution of 1-((2-aminopyridin-4-yl)oxy)propan-2-ol (300 mg, 1.0 eq) in EtOH (8 mL) was added triethylamine (542 mg, 3.0 eq) and 571-4 (537 mg, 2 eq). The mixture was stirred at 80° C. for 3 hours. The reaction mixture was diluted with H₂O (30 mL) and extracted with ethyl acetate (40 mL×3). The combined organic layers were washed with brine (20 mL×3), dried over Na₂SO₄, filtered and concentrated under reduced pressure to give a residue. The residue was purified by column chromatography (SiO₂, petroleum ether/ethyl acetate=5/1 to 0/1) to give ethyl 7-(2-hydroxypropoxy)imidazo[1,2-a]pyridine-3-carboxylate (100 mg) as a white solid. MS (ESI): m/z for C₁₃H₁₆N₂O₄ [M+H]+ calcd. 265.2 [M+H]+ found: 265.2.

Step 3: To a solution of 571-7 (190 mg, 1.1 eq) in toluene (1.5 mL) was added AlMe₃ (2 M, 2.5 eq), then reaction mixture was stirred at 25° C. for 0.5 hour. Ethyl 7-(2-hydroxypropoxy)imidazo[1,2-a]pyridine-3-carboxylate (90 mg, 1.0 eq) was added and the reaction mixture was stirred at 80° C. for 2 hours. The reaction mixture was quenched with aqueous NH₄Cl (20 mL) and then was extracted with DCM (25 mL×2). The combined organic layers were washed with brine (25 mL×2), dried over Na₂SO₄, filtered and concentrated under reduced pressure to give a residue. The residue was purified by column chromatography (SiO₂, petroleum ether/ethyl acetate=10/1 to 0/1) to give N-(5-(5-((S)-3,3-difluoro-2-hydroxypropyl)-1,2,4-oxadiazol-3-yl)-2-methylphenyl)-7-(2-hydroxypropoxy)imidazo[1,2-a]pyridine-3-carboxamide (130 mg) as a white solid. MS (ESI): m/z for C₂₃H₂₃F₂N₅O₅[M+H]+ calcd. 726.3 [M+H]+ found: 726.3.

Step 4: To a solution of N-(5-(5-((S)-3,3-difluoro-2-hydroxypropyl)-1,2,4-oxadiazol-3-yl)-2-methylphenyl)-7-(2-hydroxypropoxy)imidazo[1,2-a]pyridine-3-carboxamide (130 mg, 1.0 eq) in THE (2 mL) was added TBAF (1 M, 1.1 eq). The mixture was stirred at 25° C. for 2 hours. The reaction mixture was diluted with H₂O (30 mL) and extracted with ethyl acetate (25 mL×4). The combined organic layers were washed with brine (15 mL×2), dried over Na₂SO₄, filtered and concentrated under reduced pressure to give a residue. The residue was purified by column chromatography (SiO₂, petroleum ether/ethyl acetate=10/1 to 0/1) to give N-(5-(5-((S)-3,3-difluoro-2-hydroxypropyl)-1,2,4-oxadiazol-3-yl)-2-methylphenyl)-7-(2-hydroxypropoxy)imidazo[1,2-a]pyridine-3-carboxamide (I-571, 47.1 mg) as a white solid. MS (ESI): m/z for C₂₃H₂₃F₂N₅O₅[M+H]+ calcd. 488.1 [M+H]+ found: 488.1. ¹H NMR (400 MHz, DMSO-d₆) δ=9.88 (s, 1H), 9.27 (d, J=7.6 Hz, 1H), 8.45 (s, 1H), 8.08 (d, J=1.2 Hz, 1H), 7.81 (dd, J=1.6, 7.8 Hz, 1H), 7.49 (d, J=8.0 Hz, 1H), 7.15 (d, J=2.4 Hz, 1H), 6.87 (dd, J=2.4, 7.6 Hz, 1H), −6.24-5.86 (m, 2H), 4.97 (d, J=4.8 Hz, 1H), −4.35-4.18 (m, 1H), −4.07-3.98 (m, 1H), −3.98-3.90 (m, 2H), −3.31-3.26 (m, 1H), −3.19-3.09 (m, 1H), 2.36 (s, 3H), 1.18 (d, J=6.4 Hz, 3H).

### Example 61—Preparation of (S)—N-(5-(5-(3,3-difluoro-2-hydroxypropyl)-1,2,4-oxadiazol-3-yl)-2-methylphenyl)-7-hydroxyimidazo[1,2-a]pyridine-3-carboxamide (I-574)

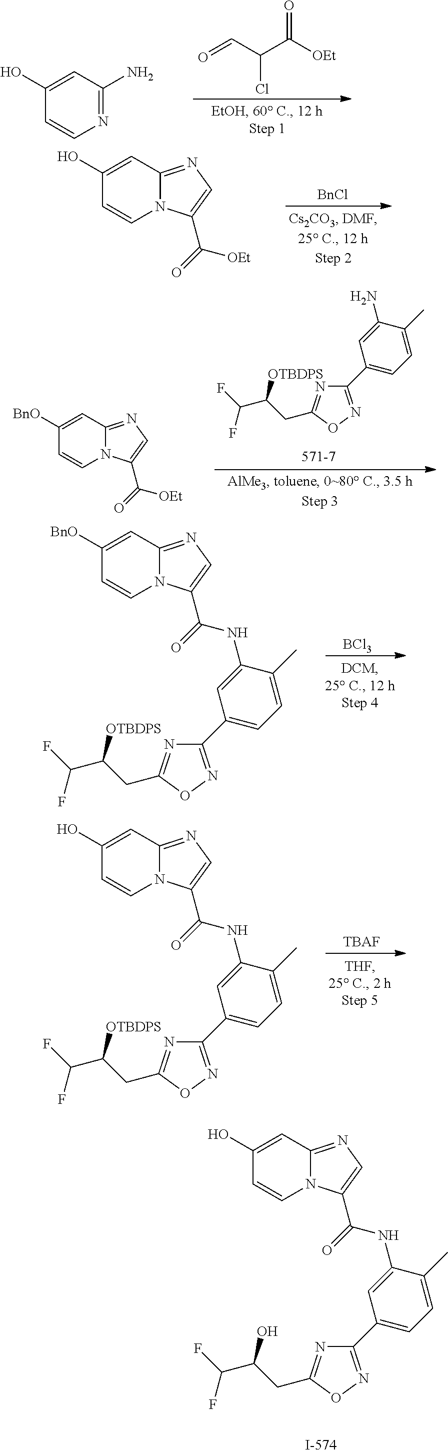

Step 1: A mixture of 2-aminopyridin-4-ol (3.0 g, 1 eq) and ethyl 2-chloro-3-oxo-propanoate (6.15 g, 1.5 eq) in EtOH (20 mL) was stirred at 60° C. for 12 hours. The mixture was cooled to 0° C. and filtered to give a brown solid. The filter cake was washed with 5 mL cold EtOH to give ethyl 7-hydroxyimidazo[1,2-a]pyridine-3-carboxylate (1.23 g, crude) as a yellow solid. ¹H NMR (400 MHz, DMSO-d₆) δ=10.91 (s, 1H), 9.00 (d, J=7.6 Hz, 1H), 8.09 (s, 1H), 6.92 (d, J=2.4 Hz, 1H), −6.88-6.83 (m, 1H), −4.34-4.26 (m, 2H), 1.31 (t, J=7.2 Hz, 3H).

Step 2: To a solution of ethyl 7-hydroxyimidazo[1,2-a]pyridine-3-carboxylate (400 mg, 1.0 eq) in DMF (5 mL) was added Cs₂CO₃ (1.26 g, 2.0 eq) and chloromethylbenzene (368 mg, 1.5 eq). The mixture was stirred at 25° C. for 12 hours. The reaction mixture was diluted with water (20 mL) and extracted with ethyl acetate (30 mL×3). The combined organic layers were washed with brine (20 mL×3), dried over Na₂SO₄, filtered and concentrated under reduced pressure to give a residue. The residue was purified by column chromatography (SiO₂, petroleum ether/ethyl acetate=2/1) to give ethyl 7-(benzyloxy)imidazo[1,2-a]pyridine-3-carboxylate (470 mg, 81% yield) as a white solid. ¹H NMR (400 MHz, DMSO-d₆) δ=9.04 (d, J=7.6 Hz, 1H), 8.15 (s, 1H), −7.55-7.26 (m, 6H), −7.04-6.96 (m, 1H), 5.25 (s, 2H), −4.38-4.29 (m, 2H), 1.32 (t, J=7.2 Hz, 3H). MS (ESI): m/z for C₁₇H₁₆N₂O₃ [M+H]⁺ calcd. 297.12, [MH]⁺ found: 297.0.

Step 3: To a solution of (S)-5-(5-(2-((tert-butyldiphenylsilyl)oxy)-3,3-difluoropropyl)-1,2,4-oxadiazol-3-yl)-2-methylaniline (343 mg, 1.0 eq) in the toluene (2 mL) was added AlMe₃ (2 M, 2.5 eq) at 0° C. The mixture was stirred at 25° C. for 0.5 hour under an N₂ atmosphere. Then ethyl 7-(benzyloxy)imidazo[1,2-a]pyridine-3-carboxylate (200 mg, 1.0 eq) was added and the mixture was stirred at 80° C. for 3 hours under an N₂ atmosphere. The reaction mixture was quenched by addition of an NH₄Cl solution (2 mL) at 0° C. and filtered. The filtrate was diluted with water (20 mL) and extracted with ethyl acetate (40 mL×3). The combined organic layers were washed with brine (30 mL×3), dried over Na₂SO₄, filtered and concentrated under reduced pressure to give a residue. The residue was purified by column chromatography (SiO₂, petroleum ether/ethyl acetate=1/4) to give (S)-7-(benzyloxy)-N-(5-(5-(2-((tert-butyldiphenylsilyl)oxy)-3,3-difluoropropyl)-1,2,4-oxadiazol-3-yl)-2-methylphenyl)imidazo[1,2-a]pyridine-3-carboxamide (450 mg, 88% yield) as a white solid. ¹H NMR (400 MHz, DMSO-d₆) δ=9.91 (s, 1H), 9.31 (d, J=7.6 Hz, 1H), 8.49 (s, 1H), 7.97 (d, J=1.6 Hz, 1H), −7.71-7.67 (m, 1H), −7.64-7.58 (m, 2H), −7.55-7.22 (m, 16H), −6.98-6.92 (m, 1H), −6.28-5.95 (m, 1H), 5.27 (s, 2H), −4.46-4.32 (m, 1H), 3.32 (s, 1H), 2.37 (s, 3H), 0.86 (s, 9H). MS (ESI): m/z for C₄₃H₄₁F₂N₅O₄Si [M+H]⁺ calcd. 758.29, [MH]⁺ found: 758.3.

Step 4: To a solution of (S)-7-(benzyloxy)-N-(5-(5-(2-((tert-butyldiphenylsilyl)oxy)-3,3-difluoropropyl)-1,2,4-oxadiazol-3-yl)-2-methylphenyl)imidazo[1,2-a]pyridine-3-carboxamide (200 mg, 1.0 eq) in DCM (2 mL) was added BCl₃ (155 mg, 5.0 eq). The mixture was stirred at 25° C. for 12 hours. The reaction mixture was quenched by addition of water (10 mL) at 25° C., and then extracted with ethyl acetate (20 mL×3). The organic layers were dried over Na₂SO₄, filtered and concentrated under reduced pressure to give a residue. The crude product was purified by reversed-phase HPLC (0.1% FA condition) to give (S)—N-(5-(5-(2-((tert-butyldiphenylsilyl)oxy)-3,3-difluoropropyl)-1,2,4-oxadiazol-3-yl)-2-methylphenyl)-7-hydroxyimidazo[1,2-a]pyridine-3-carboxamide (90 mg, 49% yield) as a white solid. MS (ESI): m/z for C₃₆H₃₅F₂N₅O₄Si [M+H]⁺ calcd. 668.24, [MH]⁺ found: 668.3.

Step 5: To a solution of (S)—N-(5-(5-(2-((tert-butyldiphenylsilyl)oxy)-3,3-difluoropropyl)-1,2,4-oxadiazol-3-yl)-2-methylphenyl)-7-hydroxyimidazo[1,2-a]pyridine-3-carboxamide (90 mg, 1.0 eq) in THE (1 mL) was added TBAF (1 M, 0.2 mL, 1.48 eq). The mixture was stirred at 25° C. for 2 hours. The reaction mixture was diluted with water (10 mL) and extracted with ethyl acetate (20 mL×3). The combined organic layers were washed with brine (10 mL×6), dried over Na₂SO₄, filtered and concentrated under reduced pressure to give a residue. The residue was purified by prep-HPLC (FA condition; column: C18 150×30 mm; mobile phase: [wateIA)-ACN]; gradient:15%-45% B over 7 min) to give (S)—N-(5-(5-(3,3-difluoro-2-hydroxypropyl)-1,2,4-oxadiazol-3-yl)-2-methylphenyl)-7-hydroxyimidazo[1,2-a]pyridine-3-carboxamide (I-574, 29.3 mg, 46% yield) as an off-white solid. ¹H NMR (400 MHz, DMSO-d₆) δ=9.80 (s, 1H), 9.24 (d, J=7.6 Hz, 1H), 8.40 (s, 1H), 8.07 (d, J=1.2 Hz, 1-), 7.82-7.77 (m, 1H), 7.48 (d, J=8.0 Hz, 1H), 6.87 (s, 1H), 6.77 (d, J=6.8 Hz, 1-), 6.23-5.87 (m, 2-), 4.31-4.19 (m, 1H), 3.27 (d, J=4.0 Hz, 1-), 3.19-3.11 (m, 2H), 2.35 (s, 3H). MS (ESI): m/z for C₂₀H₁₇F₂N₅O₄[M+H]+ calcd. 430.12, [MH]⁺ found: 430.0.

### Example 62—Preparation of (S)-7-(((1H-1,2,3-triazol-4-yl)methoxy)methyl)-N-(5-(5-(3,3-difluoro-2-hydroxypropyl)-1,2,4-oxadiazol-3-yl)-2-methylphenyl)imidazo[1,2-a]pyridine-3-carboxamide (I-575)

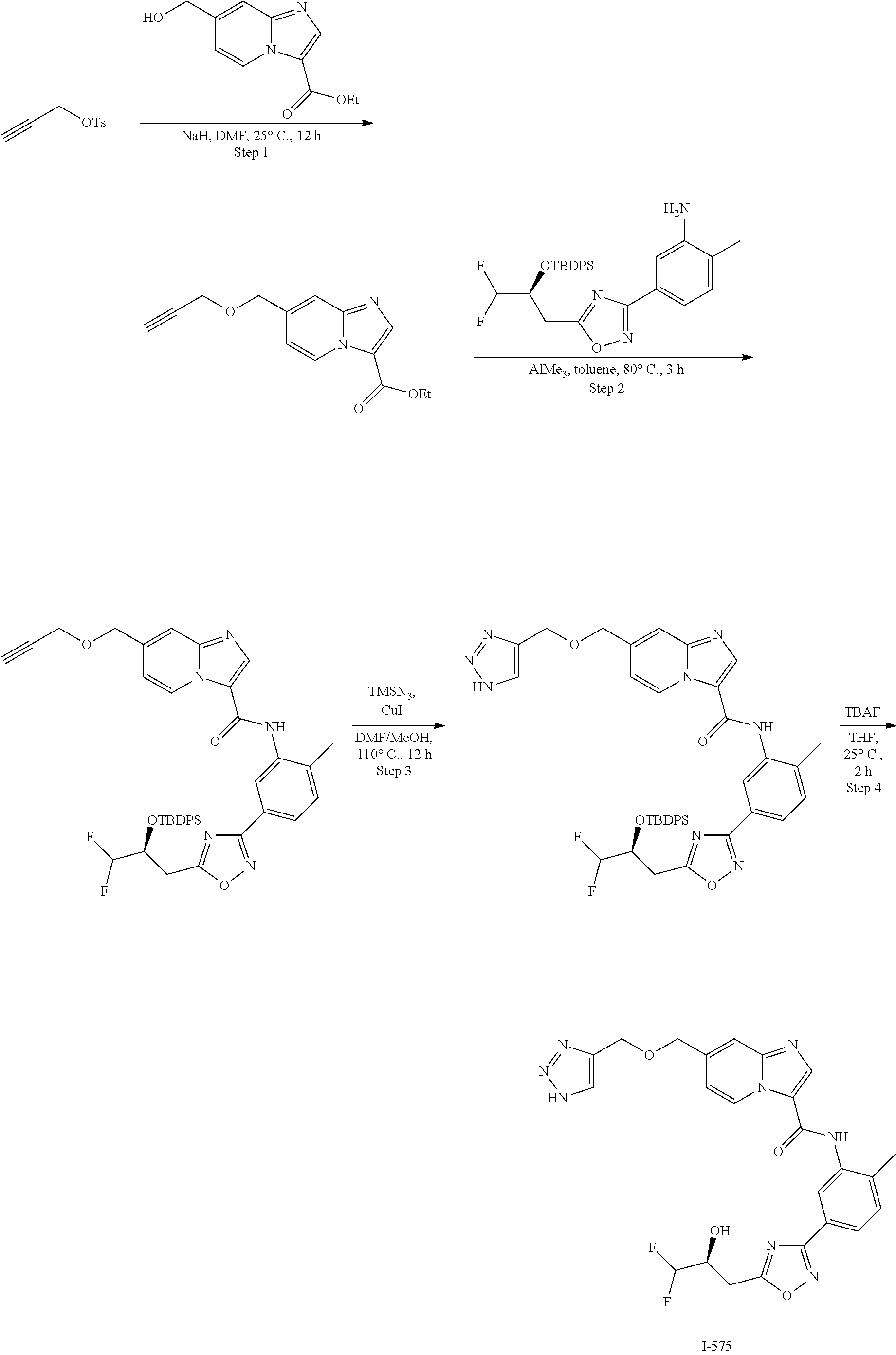

Step 1: To a solution of ethyl 7-(hydroxymethyl)imidazo[1,2-a]pyridine-3-carboxylate (1.0 g, 1.0 eq) in DMA (10 mL) was added NaH (272 mg, 60% purity, 1.5 eq) under N₂. The mixture was stirred at 25° C. for 0.5 hour. To the mixture was added prop-2-ynyl 4-methylbenzenesulfonate (1.0 g, 1.05 eq) and NaI (69 mg, 0.1 eq). The mixture was stirred at 60° C. for 12 hours. The mixture was cooled to rt. To the mixture was added 20 mL of NH₄Cl (aq.) and extracted with ethyl acetate (30 mL×3). The combined organic layers were washed with brine (20 mL×3), dried over Na₂SO₄, filtered and concentrated under reduced pressure to give a residue. The residue was purified by column chromatography (SiO₂, petroleum ether/ethyl acetate=3/1 to 0/1) to give ethyl 7-((prop-2-yn-1-yloxy)methyl)imidazo[1,2-a]pyridine-3-carboxylate (140 mg, 11% yield) as yellow solid. MS (ESI): m/z for: C₁₄H₁₄N₂O₃ [M+H]⁺ calcd. 259.1 [M+H]⁺ found: 259.1. ¹H NMR (400 MHz, chloroform-d) δ=9.25 (d, J=7.2 Hz, 1H), 8.28 (s, 1H), 7.69 (s, 1H), 7.05 (d, J=7.2 Hz, 1H), 4.70 (s, 2-), 4.48-4.34 (m, 2H), 4.25 (d, J=1.6 Hz, 2H), 2.51 (t, J=2.0 Hz, 1H), 1.42 (t, J=7.2 Hz, 3H).

Step 2: To a solution of (S)-5-(5-(2-((tert-butyldiphenylsilyl)oxy)-3,3-difluoropropyl)-1,2,4-oxadiazol-3-yl)-2-methylaniline (255 mg, 1.0 eq) in toluene (2 mL) was added Al(CH₃)₃ (2 M, 2.5 eq) and the reaction mixture was stirred at 25° C. for 0.5 hour. To the mixture was added ethyl 7-((prop-2-yn-1-yloxy)methyl)imidazo[1,2-a]pyridine-3-carboxylate (130 mg, 1.0 eq). The mixture was stirred at 80° C. for 3 hours. The mixture was poured into 20 mL of ice water and extracted with DCM (20 ml×3). The combined organic layers were washed with brine 20 mL, dried over Na₂SO₄, filtered and concentrated under reduced pressure to give a residue. The residue was purified by column chromatography (SiO₂, petroleum ether/ethyl acetate=3/1 to 1/1) to give (S)—N-(5-(5-(2-((tert-butyldiphenylsilyl)oxy)-3,3-difluoropropyl)-1,2,4-oxadiazol-3-yl)-2-methylphenyl)-7-((prop-2-yn-1-yloxy)methyl)imidazo[1,2-a]pyridine-3-carboxamide (280 mg, 74% yield) as yellow oil. MS (ESI): m/z for: C₄₀H₃₉F₂N₅O₄Si [M+H]⁺ calcd. 720.3 [M+H]⁺ found: 720.4. ¹H NMR (400 MHz, DMSO-d₆) δ=10.01 (s, 1-), 9.50-9.39 (m, 1H), 8.60 (s, 1H), 7.98 (s, 1-), 7.74-7.65 (m, 2-), 7.64-7.56 (m, 2-), 7.52-7.38 (m, 6H), 7.32 (m, 3-), 7.18-7.09 (m, 1-), 6.28-5.91 (m, 1H), 4.67 (s, 2H), 4.30 (s, 1H), 4.29 (d, J=1.6 Hz, 2H), 2.38 (s, 3H), 0.86 (s, 9H)

Step 3: To a solution of (S)—N-(5-(5-(2-((tert-butyldiphenylsilyl)oxy)-3,3-difluoropropyl)-1,2,4-oxadiazol-3-yl)-2-methylphenyl)-7-((prop-2-yn-1-yloxy)methyl)imidazo[1,2-a]pyridine-3-carboxamide (190 mg, 1.0 eq) in DMF (4 mL) and MeOH (0.4 mL) was added CuI (10 mg, 0.2 eq) and TMSN₃ (60 mg, 2.0 eq) under N₂. The mixture was stirred at 110° C. for 12 hours. To the mixture was added 50 mL of water and green solid was separated out. The solid was dried with high vacuum to give (S)-7-(((1H-1,2,3-triazol-4-yl)methoxy)methyl)-N-(5-(5-(2-((tert-butyldiphenylsilyl)oxy)-3,3-difluoropropyl)-1,2,4-oxadiazol-3-yl)-2-methylphenyl)imidazo[1,2-a]pyridine-3 carboxamide (200 mg, crude) as green solid. MS (ESI): m/z for: C₄₀H₄₀F₂N₈O₄Si [M+H]⁺ calcd. 763.3 [M+H]⁺ found: 763.4.

Step 4: To a solution of (S)-7-(((1H-1,2,3-triazol-4-yl)methoxy)methyl)-N-(5-(5-(2-((tert-butyldiphenylsilyl)oxy)-3,3-difluoropropyl)-1,2,4-oxadiazol-3-yl)-2-methylphenyl)imidazo[1,2-a]pyridine-3 carboxamide (200 mg, 1.0 eq) in THE (3 mL) was added TBAF (1 M, 314 μL, 1.2 eq). The mixture was stirred at 25° C. for 2 hours. The mixture was concentrated to give a residue. The residue was added to 10 mL of MeOH, filtered and the solid was further purified by prep-HPLC (column: C18 150×30 mm; mobile phase: [wIr(FA)-ACN]; gradient: 20%-50% B over 7 min) and prep-HPLC (column: Waters Xbridge 150*25 mm*5 um; mobile phase: [water (ammonia hydIide v/v)-ACN]; gradient:0%-25% B over 10 min) to give (S)-7-(((1H-1,2,3-triazol-4-yl)methoxy)methyl)-N-(5-(5-(3,3-difluoro-2-hydroxypropyl)-1,2,4-oxadiazol-3-yl)-2-methylphenyl)imidazo[1,2-a]pyridine-3-carboxamide (I-575, 12.89 mg, 9% yield) as white solid. MS (ESI): m/z for: C₂₄H₂₂F₂N₈O₄[M+H]⁺ calcd. 525.2 [M+H]⁺ found: 525.3. ¹H NMR (400 MHz, DMSO-d₆) δ=10.03 (s, 1H), 9.43 (d, J=7.2 Hz, 1H), 8.59 (s, 1H), 8.12 (d, J=1.6 Hz, 1H), 7.94-(s, 1H), 7.87-7.82 (m, 1H), 7.73 (s, 1H), 7.52 (d, J=8.0-Hz, 1H), 7.19-7.13-(m, 1H), 6.25-5.89 (m, 2H), 4.71-(s, 1H), 4.75-4.67-(m, 1H), 4.35-4.21 (m, 1H), 3.30-3.29-(s, 1H), 3.20-3.12 (m, 1H), 2.39 (s, 3H).

### Example 63—Preparation of (S)—N-(5-(5-(3,3-difluoro-2-hydroxypropyl)-1,2,4-oxadiazol-3-yl)-2-methylphenyl)-7-hydroxyimidazo[1,2-a]pyridine-3-carboxamide (I-576)

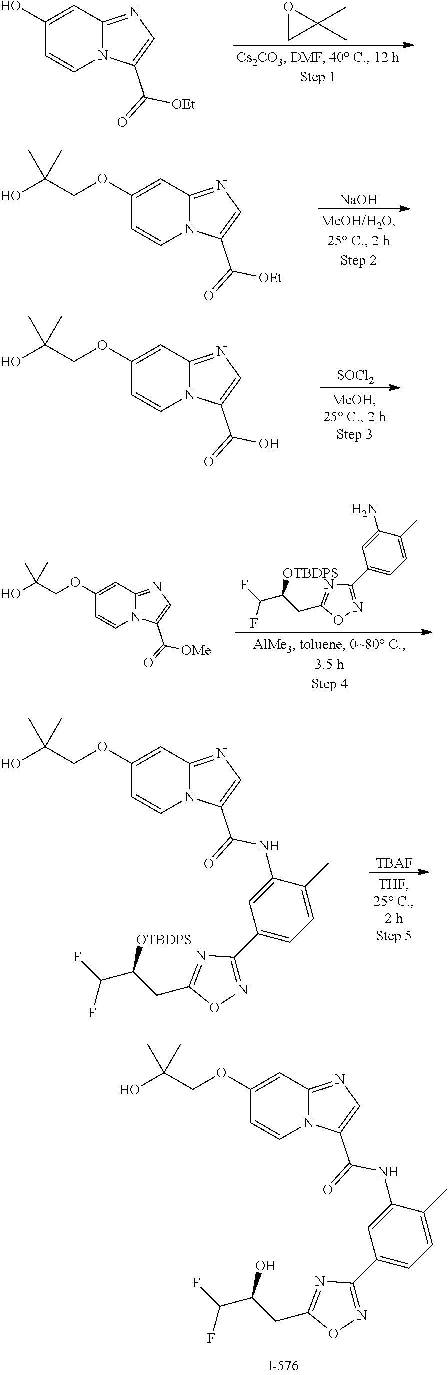

Step 1: To a solution of ethyl 7-hydroxyimidazo[1,2-a]pyridine-3-carboxylate (200 mg, 1.0 eq) in DMF (3 mL) was added Cs₂CO₃ (632 mg, 2.0 eq) and 2,2-dimethyloxirane (350 mg, 5.0 eq). The mixture was stirred at 40° C. for 12 hours. The crude product ethyl 7-(2-hydroxy-2-methylpropoxy)imidazo[1,2-a]pyridine-3-carboxylate (260 mg, crude) in DMF (3 mL) was obtained as a yellow liquid and used for the next step directly.

Step 2: To a solution of ethyl 7-(2-hydroxy-2-methylpropoxy)imidazo[1,2-a]pyridine-3-carboxylate (260 mg, 1.0 eq) in MeOH (2 mL) and H₂O (2 mL) was added NaOH (112 mg, 3.0 eq). The mixture was stirred at 25° C. for 2 hours. The reaction mixture was poured into water (3 ml), and then the pH of the solution was adjusted to 5 with 1N HCl (5 ml). The crude product was purified by reversed-phase HPLC (0.1% FA condition) to give 7-(2-hydroxy-2-methylpropoxy)imidazo[1,2-a]pyridine-3-carboxylic acid (125 mg, 41% yield) as a white solid. ¹H NMR (400 MHz, DMSO-d₆) δ=9.08 (d, J=7.6 Hz, 1H), 8.09 (s, 1H), 7.14 (d, J=2.0-Hz, 1H), 6.95-6.90-(m, 1H), 4.84-4.59 (m, 1H), 3.86 (s, 2H), 1.22 (s, 6H). MS (ESI): m/z for C₁₂H₁₄N₂O₄ [M+H]⁺ calcd. 251.10, [MH]⁺ found. 251.0.

Step 3: To a solution of 7-(2-hydroxy-2-methylpropoxy)imidazo[1,2-a]pyridine-3-carboxylic acid (100 mg, 1.0 eq) in MeOH (2 mL) was added SOCl₂ (475 mg, 10.0 eq). The mixture was stirred at 25° C. for 2 hours. The reaction mixture was concentrated under reduced pressure to remove MeOH and SOCl₂. The crude product was purified by reversed-phase HPLC (0.1% FA condition) to give methyl 7-(2-hydroxy-2-methylpropoxy)imidazo[1,2-a]pyridine-3-carboxylate (40 mg, 38% yield) as a white solid. ¹H NMR (400 MHz, DMSO-d₆) δ=9.04 (d, J=7.6 Hz, 1H), 8.17 (s, 1H), 7.18 (d, J=2.4-Hz, 1H), 6.98-6.94 (m, 1H), 4.72 (s, 1H), 3.87 (s, 2H), 3.85 (s, 3H), 1.22 (s, 6H). MS (ESI): m/z for C₁₃H₁₆N₂O₄ [M+H]⁺ calcd. 265.11, [MH]⁺ found. 265.0.

Step 4: To a solution of (S)-5-(5-(2-((tert-butyldiphenylsilyl)oxy)-3,3-difluoropropyl)-1,2,4-oxadiazol-3-yl)-2-methylaniline (77 mg, 1.0 eq) in the toluene (2 mL) was added AlMe₃ (2 M, 2.5 eq) at 0° C. The mixture was stirred at 25° C. for 0.5 hour under an N₂ atmosphere. Then methyl 7-(2-hydroxy-2-methylpropoxy)imidazo[1,2-a]pyridine-3-carboxylate (40 mg, 1.0 eq) was added and the mixture was stirred at 80° C. for 3 hours under an N₂ atmosphere. The reaction mixture was quenched by the addition of an NH₄Cl solution (2 mL) at 0° C., then was diluted with water (20 mL) and extracted with DCM (40 mL×3). The combined organic layers were washed with brine (30 mL×2), dried over Na₂SO₄, filtered and concentrated under reduced pressure to give (S)—N-(5-(5-(2-((tert-butyldiphenylsilyl)oxy)-3,3-difluoropropyl)-1,2,4-oxadiazol-3-yl)-2-methylphenyl)-7-(2-hydroxy-2-methylpropoxy)imidazo[1,2-a]pyridine-3-carboxamide (70 mg, crude) as a yellow solid. MS (ESI): m/z for C₄₀H₄₃F₂N₅O₅Si [M+H]⁺ calcd. 740.30, [MH]⁺ found: 740.4.

Step 5: To a solution of (S)—N-(5-(5-(2-((tert-butyldiphenylsilyl)oxy)-3,3-difluoropropyl)-1,2,4-oxadiazol-3-yl)-2-methylphenyl)-7-(2-hydroxy-2-methylpropoxy)imidazo[1,2-a]pyridine-3-carboxamide (60 mg, 1.0 eq) in THE (1 mL) was added TBAF (1 M, 0.2 mL, 2.47 eq). The mixture was stirred at 25° C. for 2 hours. The reaction mixture was diluted with water (10 mL) and extracted with ethyl acetate (20 mL×3). The combined organic layers were washed with brine (10 mL×6), dried over Na₂SO₄, filtered and concentrated under reduced pressure to give a residue. The residue was purified by prep-HPLC (FA condition; column: YMC-Actus Triart C18 150*30 mm*7 um; mobile phaI [water(FA)-ACN]; gradient:18%-48% B over 10 min) to give (S)—N-(5-(5-(3,3-difluoro-2-hydroxypropyl)-1,2,4-oxadiazol-3-yl)-2-methylphenyl)-7-hydroxyimidazo[1,2-a]pyridine-3-carboxamide (I-576, 29.78 mg, 67% yield) as a white solid. ¹H NMR (400 MHz, DMSO-d₆) δ=9.89 (s, 1H), 9.28 (d, J=−0.6 Hz, 1H), 8.48-8.42 (m, 1H), 8.08 (d, J=−0.6 Hz, 1H), 7.83-7.78 (m, 1H), 7.49 (d, J=8.0 Hz, 1H), 7.13 (d, J=-.4 Hz, 1H), 6.91-6-86 (m, 1H), 6.23-5.88 (m, 2H), 4-73 (s, 1H), 4.32-4.18 (m, 1H), 3.87 (s, 2H), 3.27 (d, J=−0.0 Hz, 1H), 3.19-3.11 (m, 1H), 2.36 (s, 3H), 1.23 (s, 6H). MS (ESI): m/z for C₂₄H₂₅F₂N₅O₅[M+H]⁺ calcd. 502.18, [MH]⁺ found. 502.2.

### Example 64—Preparation of (S)—N-(5-(5-(3,3-difluoro-2-hydroxypropyl)-1,2,4-oxadiazol-3-yl)-2-methylphenyl)-7-((4-hydroxy-4-methylpentyl)oxy)imidazo[1,2-a]pyridine-3-carboxamide (1-580)

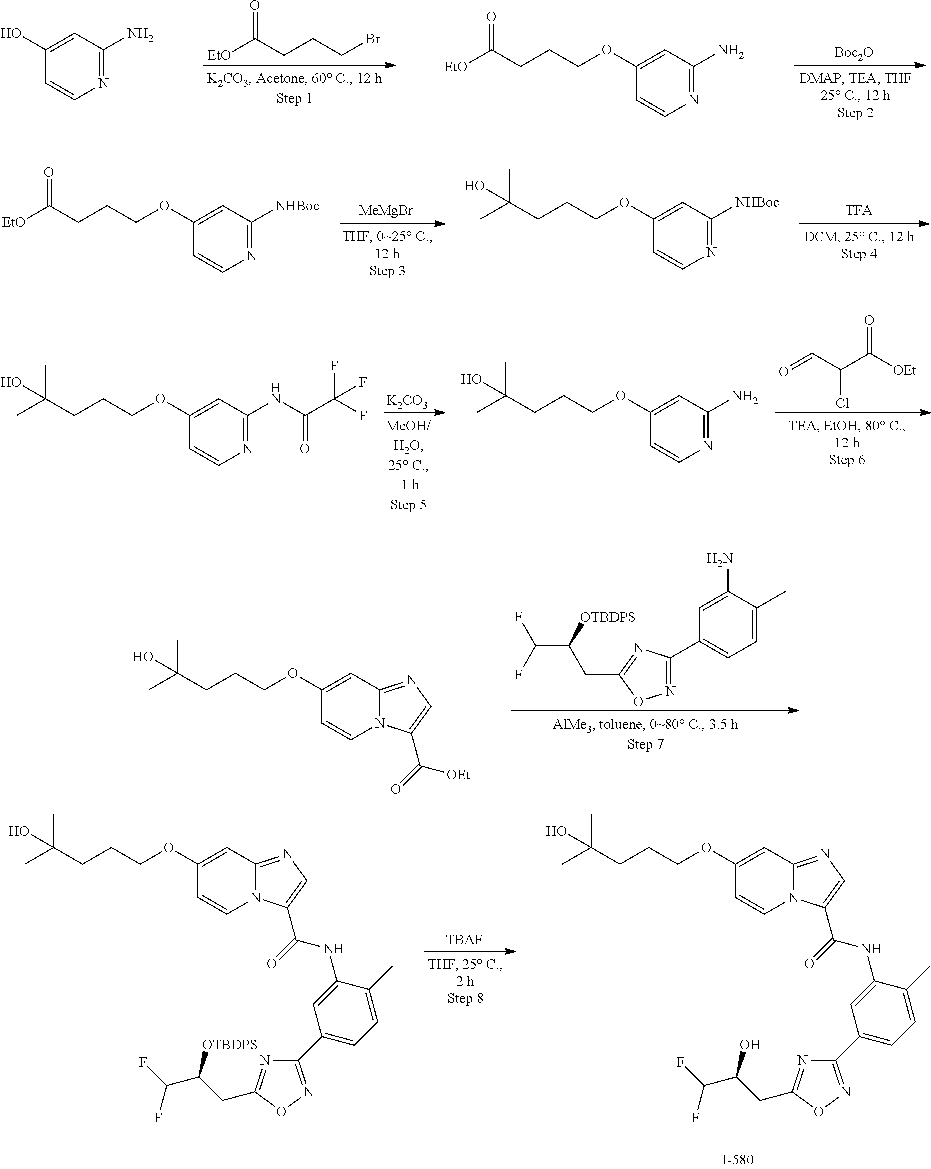

Step 1: To a solution of 2-aminopyridin-4-ol (5 g, 1.0 eq) in acetone (70 mL) was added K₂CO₃ (18.83 g, 3.0 eq) and ethyl 4-bromobutanoate (10.63 g, 1.2 eq). The mixture was stirred at 60° C. for 12 hours. The reaction mixture was filtered and concentrated under reduced pressure to give a residue. The residue was purified by column chromatography (SiO₂, EA/EtOH=10/1) to give ethyl 4-((2-aminopyridin-4-yl)oxy)butanoate (1.4 g, 14% yield) as a white solid. MS (ESI): m/z for C₁₁H₁₆N₂O₃ [M+H]⁺ calcd. 225.12, [M+H]⁺ found: 225.1.

Step 2: To a solution of ethyl 4-((2-aminopyridin-4-yl)oxy)butanoate (1.1 g, 1.0 eq) in THF (10 mL) was added Boc₂O (1.61 g, 1.5 eq), DMAP (60 mg, 0.1 eq), and triethylamine (993 mg, 2.0 eq). The mixture was stirred at 25° C. for 12 hours. The reaction was purified directly without work-up by column chromatography (SiO₂, petroleum ether/ethyl acetate=2/3) to give ethyl 4-((2-((tert-but543midazolonyl)amino)pyridin-4-yl)oxy)butanoate (1.25 g, 53% yield) as a white solid. ¹H NMR (400 MHz, DMSO-d₆) δ=9.71 (s, 1H), 8.22 (d, J=5.6 Hz, 1H), 8.03 (d, J=5.6 Hz, 1H), 7.38 (d, J=2.0 Hz, 1H), 6.94 (d, J=−0.0 Hz, 1H), 6.92-6-88 (m, 1H), 6.62-6-58 (m, 1H), 4.12-4.02 (m, 8H), 2.45 (t, J=−0.2 Hz, 4H), 2.02-1.93 (m, 4H), 1.46 (s, 9H), 1.-9 (s, 16H), 1.42-1-34 (m, 1H), 1.20-1.14 (m, 6H). MS (ESI): m/z for C₁₆H₂₄N₂O₅ [M+H]⁺ calcd. 325.17, [M+H]⁺ found. 325.1

Step 3: To a solution of ethyl 4-((2-((tert-but543midazolonyl)amino)pyridin-4-yl)oxy)butanoate (600 mg, 1.0 eq) in THE (6 mL) was added MeMgBr (3 M, 5.0 eq) at 0° C. under an N₂ atmosphere. The mixture was stirred at 25° C. for 12 hours. The reaction mixture was quenched by addition of an NH₄Cl solution (5 mL) at 0° C., and then diluted with water (20 mL) and extracted with ethyl acetate (40 mL×3). The organic layer was washed with brine (40 mL×3), dried over Na₂SO₄, filtered and concentrated under reduced pressure to give tert-butyl (4-((4-hydroxy-4543midazolpentyl)oxy)pyridin-2-yl)carbamate (490 mg, 77% yield) as a white solid. ¹H NMR (400 MHz, DMSO-d₆) δ=9.69 (s, 1H), 8.03 (d, J=6.0 Hz, 1H), 7.37 (d, J=−0.4 Hz, 1H), 6.62-6.58 (m, 1H), 4.18 (s, 1H), 4.01 (t, J=−0.4 Hz, 2H), 1.81-1.71 (m, 2H), 1.46 (s, 11H), 1.10 (s, 6H). MS (ESI): m/z for C₁₆H₂₆N₂O₄ [M+H]⁺ calcd. 311.19, [MH]⁺ found: 311.1.

Step 4: To a solution of tert-butyl (4-((4-hydroxy-4543midazolpentyl)oxy)pyridin-2-yl)carbamate (250 mg, 1.0 eq) in DCM (2 mL) was added TFA (1 mL). The mixture was stirred at 25° C. for 12 hours. The reaction mixture was concentrated under reduced pressure to remove DCM and TFA to give 2,2,2-trifluoro-N-(4-((4-hydroxy-4543midazolpentyl)oxy)pyridin-2-yl)acetamide (246 mg, crude) as a black oil.

Step 5: To a solution of 2,2,2-trifluoro-N-(4-((4-hydroxy-4543midazolpentyl)oxy)pyridin-2-yl)acetamide (246 mg, 1.0 eq) in MeOH (1.5 mL) and H₂O (1.5 mL) was added K₂CO₃ (222 mg, 2.0 eq). The mixture was stirred at 25° C. for 1 hour. The reaction mixture was diluted with water (20 mL) and extracted with ethyl acetate (40 mL×3). The organic layer was washed with brine (40 mL×3), dried over Na₂SO₄, filtered and concentrated under reduced pressure to give 5-((2-aminopyridin-4-yl)oxy)-2-methylpentan-2-ol (140 mg, 51% yield) as a yellow solid. MS (ESI): m/z for C₁₁H₁₈N₂O₂ [M+H]⁺ calcd. 211.14, [MH]⁺ found: 211.1.

Step 6: To a solution of 5-((2-aminopyridin-4-yl)oxy)-2-methylpentan-2-ol (140 mg, 1.0 eq) in EtOH (2 mL) was added triethylamine (202 mg, 3.0 eq) and ethyl 2-chloro-3-oxo-propanoate (120 mg, 1.2 eq). The mixture was stirred at 80° C. for 12 hours. The reaction was purified directly without work-up. The crude product was purified by reversed-phase HPLC(0.1% FA condition) to give ethyl 7-((4-hydroxy-4-methylpentyl)oxy)imidazo[1,2-a]pyridine-3-carboxylate (90 mg, 31% yield) as a yellow solid. ¹H NMR (400 MHz, DMSO-d₆) δ=9.02 (d, J=7.6 Hz, 1H), 8.14 (s, 1H), 7.16 (d, J=−0.4 Hz, 1H), 6.94-6-90 (m, 1H), 4.36-4.29 (m, 2H), 4.21 (s, 1H), 4.10 (t, J=−0.4 Hz, 2H), 1.86-1-76 (m, 2H), 1.55-1.47 (m, 2H), 1.32 (t, J=7.2 Hz, 3H), 1.11 (s, 6H). MS (ESI): m/z for C₁₆H₂₂N₂O₄ [M+H]⁺ calcd. 307.16, [MH]⁺ found: 307.0.

Step 7: To a solution of (S)-5-(5-(2-((tert-butyldiphenylsilyl)oxy)-3,3-difluoropropyl)-1,2,4-oxadiazol-3-yl)-2-methylaniline (149 mg, 1.0 eq) in the toluene (1 mL) was added AlMe₃ (2 M, 2.5 eq) at 0° C. The mixture was stirred at 25° C. for 0.5 hours under an N₂ atmosphere. Then ethyl 7-((4-hydroxy-4-methylpentyl)oxy)imidazo[1,2-a]pyridine-3-carboxylate (90 mg, 1.0 eq) was added and the mixture was stirred at 80° C. for 3 hours under an N₂ atmosphere. The reaction mixture was quenched by the addition of an NH₄Cl solution (2 mL) at 0° C., then was diluted with water (20 mL) and extracted with DCM (40 mL×3). The combined organic layers were washed with brine (30 mL×2), dried over Na₂SO₄, filtered and concentrated under reduced pressure to give (S)—N-(5-(5-(2-((tert-butyldiphenylsilyl)oxy)-3,3-difluoropropyl)-1,2,4-oxadiazol-3-yl)-2-methylphenyl)-7-((4-hydroxy-4-methylpentyl)oxy)imidazo[1,2-a]pyridine-3-carboxamide (150 mg, crude) as a yellow oil. MS (ESI): m/z for C₄₂H₄₇F₂N₅O₅Si [M+H]⁺ calcd. 768.33, [MH]+ found: 768.4.

Step 8: To a solution of (S)—N-(5-(5-(2-((tert-butyldiphenylsilyl)oxy)-3,3-difluoropropyl)-1,2,4-oxadiazol-3-yl)-2-methylphenyl)-7-((4-hydroxy-4-methylpentyl)oxy)imidazo[1,2-a]pyridine-3-carboxamide (140 mg, 1.0 eq) in THE (2 mL) was added TBAF (1 M, 2.0 eq). The mixture was stirred at 25° C. for 2 hours. The reaction mixture was diluted with water (10 mL) and extracted with ethyl acetate (20 mL×3). The combined organic layers were washed with brine (10 mL×6), dried over Na₂SO₄, filtered and concentrated under reduced pressure to give a residue. The residue was purified by prep-HPLC (basic condition, column: Waters Xbridge 150*25 mm*5 um; mobile phase: [water (ammonIhydroxide v/v)-ACN]; gradient:20%-500% B over 10 min) to give (S)—N-(5-(5-(3,3-difluoro-2-hydroxypropyl)-1,2,4-oxadiazol-3-yl)-2-methylphenyl)-7-((4-hydroxy-4-methylpentyl)oxy)imidazo[1,2-a]pyridine-3-carboxamide (I-580, 42.29 mg, 44% yield) as a white solid. ¹H NMR (400 MHz, DMSO-d₆) δ=9.88 (s, 1H), 9.26 (d, J=7.6 Hz, 1H), 8.45 (s, 1H), 8.08 (d, J-=1.6 Hz, 1H), 7.83-7.79 (m, 1H), 7.48 (d, J=8.0 Hz, 1H), 7.13 (d, J-=2.4 Hz, 1H), 6.88-6.83 (m, 1H), 6.21-5.90 (m, 2H), 4.32-4.18 (m, 2H), 4.10 (t, J-=6.4 Hz, 2H), 3.32-3.25 (m, 1H), 3.20-3.10 (m, 1H)-2.36 (s, 3H), 1.87-1.77 (m, 2H), 1.55-1.47 (m, 2H), 1.12 (s, 6H). MS (ESI): m/z for C₂₆H₂₉F₂N₅O₅[M+H]⁺ calcd. 530.2, [M+H]⁺ found: 530.3.

### Example 65—Preparation of 7-methoxy-N-(2-methyl-5-(5-((tetrahydrofuran-2-yl)methyl)-1,2,4-oxadiazol-3-yl)phenyl)imidazo[1,2-a]pyridine-3-carboxamide

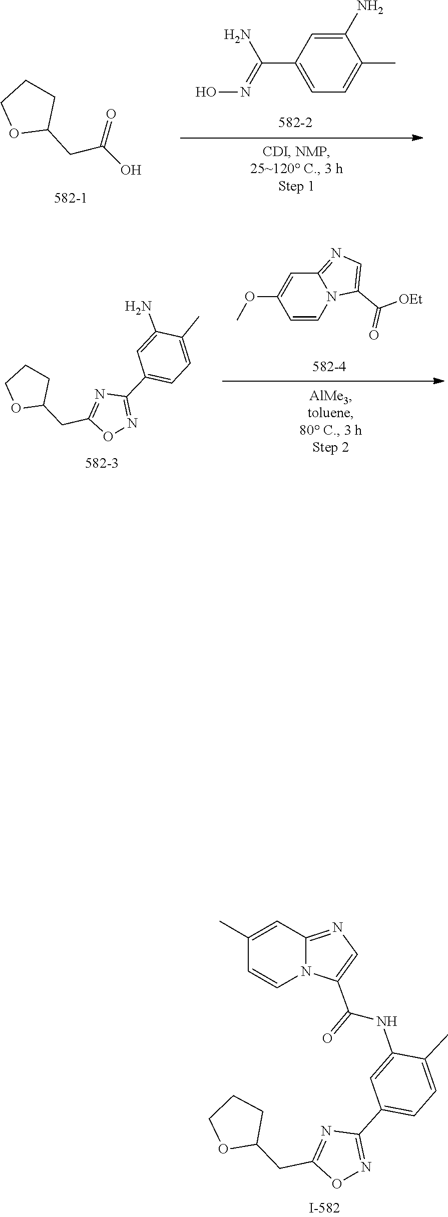

Step 1: To a solution of 582-1 (100 mg, 1.0 eq) in NMP (1 mL) was added CDI (137 mg, 1.1 eq). The mixture was stirred at 25° C. for 0.5 hour. Then 582-2 (115 mg, 0.9 eq) was added. The mixture was stirred at 120° C. for 3 hours. The reaction mixture was diluted with H₂O (30 mL) and extracted with ethyl acetate (40 mL×2). The combined organic layers were washed with brine (20 mL×3), dried over Na₂SO₄, filtered and concentrated under reduced pressure to give a residue. The residue was purified by column chromatography (SiO₂, petroleum ether/ethyl acetate=5/1 to 0/1) to give 2-methyl-5-(5-((tetrahydrofuran-2-yl)methyl)-1,2,4-oxadiazol-3-yl) aniline (90 mg) as a white solid. ¹H NMR (400 MHz, methanol-d₄) δ=7.39 (d, J-=1.6 Hz, 1H), 7.31-7.26 (m, 1H), 7.11 (d, J=7.6 Hz, 1H), 4.4-4.35 (m, 1H), 3.91-3.70 (m, 2H), 3.16 (d, J=6.4 Hz, 2H)-2.21 (s, 3H), 2.19-2.11 (m, 1H), 2.04-1.89 (m, 2H), 1.82-1.71 (m, 1H).

Step 2: To a solution of 2-methyl-5-(5-((tetrahydrofuran-2-yl)methyl)-1,2,4-oxadiazol-3-yl) aniline (70 mg, 1.0 eq) in toluene (1 mL) was added AlMe₃ (2 M, 2.5 eq) and the reaction mixture was stirred at 25° C. for 0.5 hour. Then 582-4 (65 mg, 1.1 eq) was added and the reaction mixture was stirred at 80° C. for 2 hours. The reaction mixture was quenched with a solution of NH₄Cl (20 mL) and then was extracted with DCM (25 mL×2). The combined organic layers were washed with brine (25 mL×2), dried over Na₂SO₄, filtered and concentrated under reduced pressure to give a residue. The residue was purified by prep-HPLC (FA condition; column: C18 150×30 mm; mobilphase: [water(FA)-ACN]; gradient:20%-50% B over 7 min) to give 7-methoxy-N-(2-methyl-5-(5-((tetrahydrofuran-2-yl)methyl)-1,2,4-oxadiazol-3-yl)phenyl)imidazo[1,2-a]pyridine-3-carboxamide (I-582, 43.23 mg) as a white solid. ¹H NMR (400 MHz, methanol-d₄) δ=9.30 (d, J=7.6 Hz, 1H), 8.33 (s, 1H), 8.09 (d, J=1.6 Hz, 1H), 7.88 (dd, J=1.6, 8.0 Hz, 1H), 7.45 (d, J=8.0 Hz, 1H), 7.04 (d, J=2.4 Hz, 1H), 6.83 (dd, J=2.4, 7.6 Hz, 1H), 4.46-4.36 (m, -H), 3.94 (s, 3H), 3-2-3.85 (m, 1H), 3.79-3.71 (m, 1H), 3.18 (d, J=6.4 Hz, —H), 2.40 (s, 3H), 2.-4-2.10 (m, 1H), 2.-3-1.88 (m, 2H), 1.82-1.69 (m, 1H). MS (ESI): m/z for C₂₃H₂₃N₅O₄ [M+H]+ calcd. 433.18, [MH]+ found: 434.2

### Example 66—Preparation of 7-methyl-N-(2-methyl-5-(5-((tetrahydro-2H-pyran-2-yl)methyl)-1,2,4-oxadiazol-3-yl)phenyl)imidazo[1,2-a]pyridine-3-carboxamide (I-583)

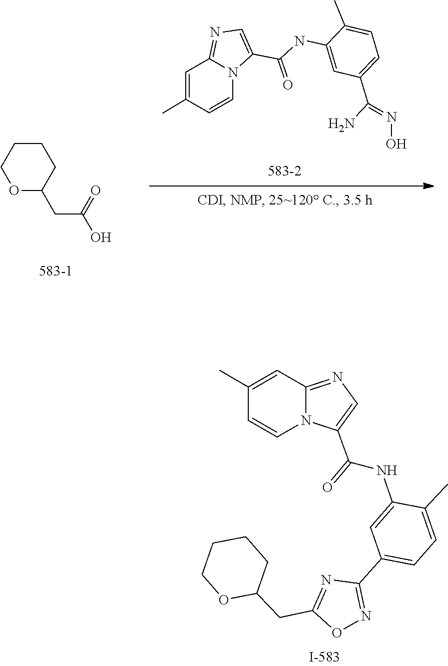

To a solution of 583-1 (50 mg, 1.0 eq) in 546midazoleL) was added di(imidazol-1-yl)methanone (67 mg, 1.2 eq). The mixture was stirred at 25° C. for 0.5 hour. The mixture was used without further work up. To the solution was added 583-2 (112 mg, 1.0 eq). The mixture was stirred at 120° C. for 3 hours. The reaction was purified directly without work-up by prep-HPLC (FA condition; column: C18 150×30 mm; mole phase: [water (FA)-ACN]; gradient: 28%-58% B over 7 min) to give 7-methyl-N-(2-methyl-5-(5-((tetrahydro-2H-pyran-2-yl)methyl)-1,2,4-oxadiazol-3-yl)phenyl)imidazo[1,2-a]pyridine-3-carboxamide (I-583, 67.43 mg, 41% yield) as a white solid. ¹H NMR (400 MHz, DMSO-d₆) δ=9.94 (s, 1H), 9.32 (d, J=7.2 Hz, 1H), 8.52 (s, 1H), 8.07-(d, J=1.2 Hz, 1H), 7.82-7.77 (m, 1H), 7.56 (s, 1H), 7.48-(d, J=8.0 Hz, 1H), −7.05-7.00 (m, 1H), −3.86-3.76 (m, 2H), −3.39-3.34 (m, 1H), 3.21-3.06 (m, 2H), 2.43 (-, 3H), 2.36 (s, 3H), −1.83-1.69 (m, 2H), 1.55-1.30 (m, 4H). MS (ESI): m/z for C₂₄H₂₅N₅O₃ [M+H]⁺ calcd. 432.20, [MH]⁺ found: 432.2.

### Example 67—Preparation of (S)-7-(((1H-imidazol-4-yl)methoxy)methyl)-N-(5-(5-(3,3-difluoro-2-hydroxypropyl)-1,2,4-oxadiazol-3-yl)-2-methylphenyl)imidazo[1,2-a]pyridine-3-carboxamide (I-585)

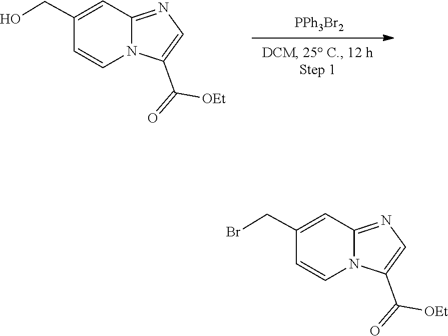 [SMILES: CCOC(=O)c1cnc2cc(CO)ccn12 ; CCOC(=O)c1cnc2cc(CBr)ccn12 ; "PPh3Br2"]

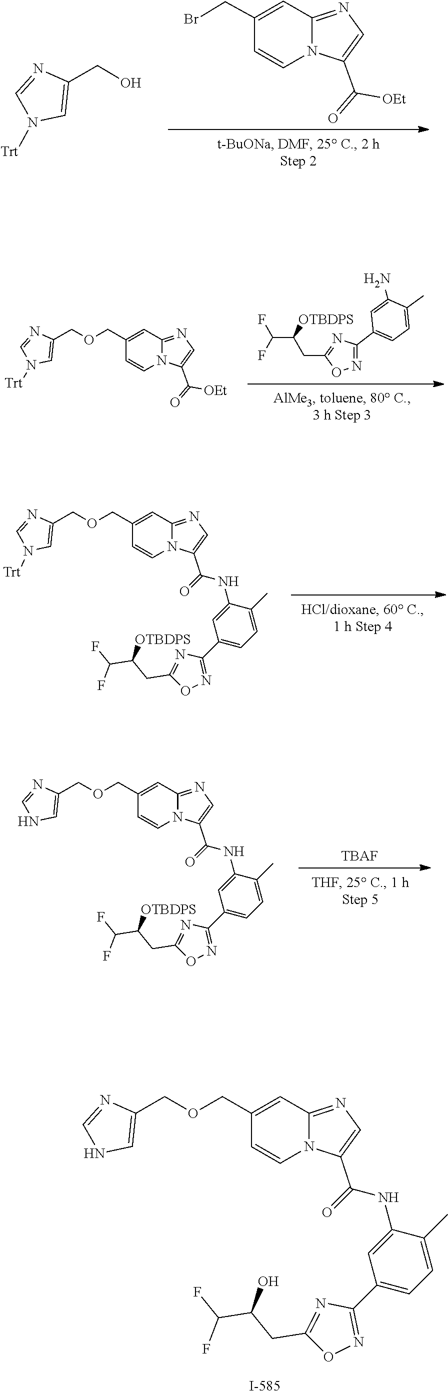

Step 1: To a solution of ethyl 7-(hydroxymethyl)imidazo[1,2-a]pyridine-3-carboxylate (5 g, 1.0 eq) in DCM (100 mL) was added dibromo(triphenyl)-phosphane (11.50 g, 1.2 eq). The mixture was stirred at 25° C. for 12 hours. The reaction mixture was concentrated under reduced pressure to remove solvent. The residue was purified by column chromatography (SiO₂, DCM/MeOH=20/1 to 10/1) to give ethyl 7-(bromomethyl)imidazo[1,2-a]pyridine-3-carboxylate (3.87 g) as a white solid. MS (ESI): m/z for C₁₁H₁₁BrN₂O₂[M+H]⁺ calcd. 283.2 [MH]⁺ found: 282.9.

Step 2: To a solution of (1-trityl-1H-imidazol-4-yl)methanol (601 mg, 1.0 eq) in DMF (5 mL) was added t-BuONa (339 mg, 2.0 eq) at 0° C. The mixture was stirred at 0° C. for 0.5 hours. Then ethyl 7-(bromomethyl)imidazo[1,2-a]pyridine-3-carboxylate (500 mg, 1.0 eq) was added. The mixture was stirred at 25° C. for 1.5 hours. The reaction mixture was partitioned between H₂O (30 mL) and ethyl acetate (50 mL×3). The organic phase was separated, washed with brine (15 mL×3), dried over Na₂SO₄, filtered and concentrated under reduced pressure to give a residue. The residue was purified by prep-HPLC (column: Phenomenex luna C18 150*40 mm*15 umIobile phase: [water(FA)-ACN]; gradient:28%-58% B over 15 min) to548midazolehyl 7-(((1-trityl-1H-imidazol-4-yl)methoxy)methyl)imidazo[1,2-a]pyridine-3-carboxylate (100 mg) as a yellow oil. MS (ESI): m/z for C₃₄H₃₀N₄O₃ [M+H]⁺ calcd. 543.6[MH]⁺ found: 301.2.

Step 3: To a solution of (S)-5-(5-(2-((tert-butyldiphenylsilyl)oxy)-3,3-difluoropropyl)-1,2,4-oxadiazol-3-yl)-2-methylaniline (94 mg, 1.0 eq) in toluene (2 mL) was added Al(CH₃)₃ (3 M, 154 μL, 2.5 eq). The mixture was stirred at 25° C. for 0.5 hour. Then ethyl 7-(((1-trityl-1H-imidazol-4-yl)methoxy)methyl)imidazo[1,2-a]pyridine-3-carboxylate (100 mg, 1.0 eq) was added. The mixture was stirred at 80° C. for 1.5 hours. The reaction mixture was quenched by the addition of a saturated solution of ammonium chloride in water (10 mL) at 0° C., and then diluted with H₂O (10 mL) and extracted with DCM (20 mL×3). The combined organic layers were washed with brine (10 mL×2), dried over Na₂SO₄ filtered and concentrated under reduced pressure to give (S)—N-(5-(5-(2-((tert-butyldiphenylsilyl)oxy)-3,3-difluoropropyl)-1,2,4-oxadiazol-3-yl)-2-methylphenyl)-7-(((1-trityl-1H-imidazol-4-yl)methoxy)methyl)imidazo[1,2-a]pyridine-3-carboxamide (220 mg, crude) as a yellow solid. MS (ESI): m/z for C₆₀H₅₅F₂N₇O₄Si [M+H]⁺ calcd. 1004.4 [MH]⁺ found: 1004.6.

Step 4: A mixture of (S)—N-(5-(5-(2-((tert-butyldiphenylsilyl)oxy)-3,3-difluoropropyl)-1,2,4-oxadiazol-3-yl)-2-methylphenyl)-7-(((1-trityl-1H-imidazol-4-yl)methoxy)methyl)imidazo[1,2-a]pyridine-3-carboxamide (200 mg, 1.0 eq) in HCl/dioxane (5 mL) was stirred at 60° C. for 1 hour. The reaction mixture was concentrated under reduced pressure to remove solvent. The residue was purified by prep-HPLC (column: C18 150×30I mobile phase: [water(FA)-ACN]; gradient:388%-68% B over 7 min) to give (S)-7-(((1H-imidazol-4-yl)methoxy)methyl)-N-(5-(5-(2-((tert-butyldiphenylsilyl)oxy)-3,3-difluoropropyl)-1,2,4-oxadiazol-3-yl)-2-methylphenyl)imidazo[1,2-a]pyridine-3-carboxamide (30 mg) as a white solid. MS (ESI): m/z for C₄₁H₄₁F₂N₇O₄Si [M+H]⁺ calcd. 762.4 [MH]⁺ found: 762.5.

Step 5: To a solution of (S)-7-(((1H-imidazol-4-yl)methoxy)methyl)-N-(5-(5-(2-((tert-butyldiphenylsilyl)oxy)-3,3-difluoropropyl)-1,2,4-oxadiazol-3-yl)-2-methylphenyl)imidazo[1,2-a]pyridine-3-carboxamide (25 mg, 1.0 eq) in THE (1 mL) was added TBAF (1 M, 1.2 eq). The mixture was stirred at 25° C. for 1 hour. The reaction mixture was partitioned between H₂O (5 mL) and ethyl acetate (5 ml×3). The organic phase was separated, washed with brine (5 mL×3), dried over Na₂SO₄, filtered and concentrated under reduced pressure to give a residue. The residue was purified by prep-HPLC (column: C18 150 Imm; mobile phase: [water(FA)-ACN]; gradient:10%-40% B over 7 min) to give (S)-7-(((1H-imidazol-4-yl)methoxy)methyl)-N-(5-(5-(3,3-difluoro-2-hydroxypropyl)-1,2,4-oxadiazol-3-yl)-2-methylphenyl)imidazo[1,2-a]pyridine-3-carboxamide (I-585, 6.54 mg) as a white solid. MS (ESI): m/z for C₂₅H₂₃F₂N₇O₄[M+H]⁺ calcd. 524.2 [MH]⁺ found: 524.1. ¹H NMR (400 MHz, DMSO-d₆) δ=9.99 (s, 1H), 9.40 (d, J=7.2 Hz, 1H), 8.57 (s, 1H), 8.15 (s, 1H), 8.09 (d, J=1.6 Hz, 1H), 7.82 (dd, J=1.6, 8.0 Hz, 1H), 7.69 (s, 1H), 7.63 (s, 1-), 7.50 (d, J=8.0-z, 1H), 7.15-7.05 (m, 2H), 6.24-5.86 (m, 2H)-4.62 (s, 2H), 4.50 (s, 2H), 4.35-4.17 (m, 1H), 3.27 (s, 1H), 3.18-3.13 (m, 1H), 2.37 (s, 3H)

### Example 68—Preparation of (S)-7-(((1H-pyrazol-5-yl)methoxy)methyl)-N-(5-(5-(3,3-difluoro-2-hydroxypropyl)-1,2,4-oxadiazol-3-yl)-2-methylphenyl)imidazo[1,2-a]pyridine-3-carboxamide (I-586)

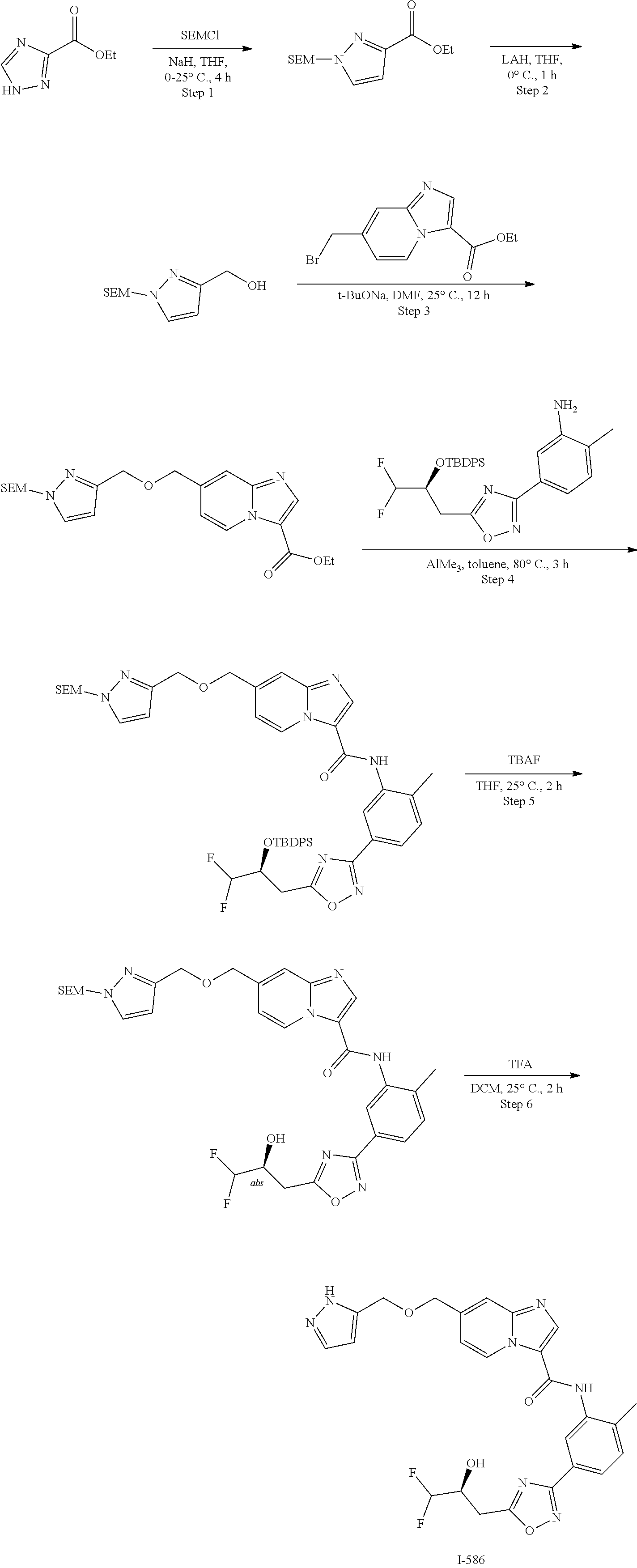

Step 1: To a solution of NaH (1.37 g, 60% purity, 1.6 eq) in THF (30 mL) was added a solution of ethyl 1H-pyrazole-3-carboxylate (3 g, 1.0 eq) in THF (30 mL). The reaction mixture was stirred at 25° C. for 1 hour, then a solution of SEM-Cl (4.28 g, 1.2 eq) in THF (15 mL) was added at 0° C. and the reaction mixture was stirred at 25° C. for 12 hours. The reaction mixture was quenched with H₂O (100 mL) and was extracted with ethyl acetate (50 mL×3). The combined organic layers were washed with brine (25 mL×4), dried over Na₂SO₄, filtered and concentrated under reduced pressure to give a residue. The residue was purified by column chromatography (SiO₂, petroleum ether/ethyl acetate=10/1 to 3/1) to give ethyl 1-((2-(trimethylsilyl)ethoxy)methyl)-1H-pyrazole-3-carboxylate (2.2 g) as a colorless oil. ¹H NMR (400 MHz, methanol-d₄) δ=7.86 (d, J=2.4 Hz, 1H), 6.84 (d, J=2.4 Hz, 1H), 5.50 (s, 2H), 4.40-4.30 (m, 2H), 3.64-3.55 (m, 2H), 1.37 (t, J=7.2 Hz, 3H), 0.93-0.85 (m, 2H), −0.03 (s, 9H)

Step 2: To a solution of ethyl 1-((2-(trimethylsilyl)ethoxy)methyl)-1H-pyrazole-3-carboxylate (1.5 g, 1.0 eq) in THE (30 mL) was added LiAlH₄ (2.5 M, 1.2 eq) at 0° C., and the mixture was stirred at 25° C. for 12 hours. The reaction mixture was quenched with H₂O (1 mL), a 15% solution of NaOH (1 mL), H₂O (3 mL), and then was extracted with ethyl acetate (50 mL×2). The combined organic layers were washed with brine (25 mL×2), dried over Na₂SO₄, filtered and concentrated under reduced pressure to give (1-((2-(trimethylsilyl)ethoxy)methyl)-1H-pyrazol-3-yl)methanol (1.3 g, crude) as a colorless oil. ¹H NMR (400 MHz, DMSO-d₆) δ=7.76 (d, J=2.4 Hz, 1H), 6.25 (d, J=2.4 Hz, 1H), 5.33 (s, 2H), 5.03 (t, J=5.6 Hz, 1H), 4.41 (d, J=5.6 Hz, 2H), 3.58-3.44 (m, 2H), 0.87-0.76 (m, 2H), −0.04 (s, 9H).

Step 3: To a solution of (1-((2-(trimethylsilyl)ethoxy)methyl)-1H-pyrazol-3-yl)methanol (400 mg, 1.0 eq) in THF (10 mL) was added t-BuONa (253 mg, 1.5 eq) and ethyl 7-(bromomethyl)imidazo[1,2-a]pyridine-3-carboxylate (496 mg, 1.0 eq). The mixture was stirred at 25° C. for 12 hours. The reaction mixture was diluted with H₂O (30 mL) and extracted with ethyl acetate (30 mL×3). The combined organic layers were washed with brine (15 mL×2), dried over Na₂SO₄, filtered and concentrated under reduced pressure to give a residue. The residue was purified by column chromatography (SiO₂, petroleum ether/ethyl acetate=5/1 to 0/1) to give ethyl 7-(((1-((2-(trimethylsilyl)ethoxy)methyl)-1H-pyrazol-3-yl)methoxy)methyl)imidazo[1,2-a]pyridine-3-carboxylate (200 mg) as a white solid. ¹H NMR (400 MHz, methanol-d₄) δ=9.27 (d, J=7.2 Hz, 1H), 8.22 (s, 1H), 7.76 (d, J=2.4 Hz, 1H), 7.68 (s, 1H), 7.18 (dd, J=1.2, 7.2 Hz, 1H), 6.45 (d, J=2.4 Hz, 1H), 5.42 (s, 2H), 4.69 (s, 2H), 4.66 (s, 2H), 4.42 (q, J=7.2 Hz, 2H), 3.59-3.50 (m, 2H), 1.41 (t, J=7.2 Hz, 3H), 0.90-0.83 (m, 2H), −0.05 (s, 9H).

Step 4: To a solution of (S)-5-(5-(2-((tert-butyldiphenylsilyl)oxy)-3,3-difluoropropyl)-1,2,4-oxadiazol-3-yl)-2-methylaniline (130 mg, 1.1 eq) in toluene (2 mL) was added AlMe₃ (2 M, 2.5 eq) and the reaction mixture was stirred at 25° C. for 0.5 hour. Then ethyl 7-(((1-((2-(trimethylsilyl)ethoxy)methyl)-1H-pyrazol-3-yl)methoxy)methyl)imidazo[1,2-a]pyridine-3-carboxylate (100 mg, 1 eq) was added and the reaction mixture was stirred at 80° C. for 2 hours. The reaction mixture was quenched with a solution of NH₄Cl (20 mL) and then was extracted with DCM (25 mL×2). The combined organic layers were washed with brine (25 mL×2), dried over Na₂SO₄, filtered and concentrated under reduced pressure to give a residue. The residue was purified by column chromatography (SiO₂, petroleum ether/ethyl acetate=5/1 to 0/1) to give (S)—N-(5-(5-(2-((tert-butyldiphenylsilyl)oxy)-3,3-difluoropropyl)-1,2,4-oxadiazol-3-yl)-2-methylphenyl)-7-(((1-((2-(trimethylsilyl)ethoxy)methyl)-1H-pyrazol-3-yl)methoxy)methyl)imidazo[1,2-a]pyridine-3-carboxamide (90 mg) as a white solid. MS (ESI): m/z for C₄₇H₅₅F₂N₇OsSi₂ [M+H]⁺ calcd. 891.38, [MH]⁺ found: 892.2.

Step 5: To a solution of (S)—N-(5-(5-(2-((tert-butyldiphenylsilyl)oxy)-3,3-difluoropropyl)-1,2,4-oxadiazol-3-yl)-2-methylphenyl)-7-(((1-((2-(trimethylsilyl)ethoxy)methyl)-1H-pyrazol-3-yl)methoxy)methyl)imidazo[1,2-a]pyridine-3-carboxamide (90 mg, 1.0 eq) in THE (1 mL) was added TBAF (1 M, 1.1 eq). The mixture was stirred at 25° C. for 1 hour. The reaction mixture was diluted with H₂O (30 mL) and extracted with ethyl acetate (30 mL×3). The combined organic layers were washed with brine (25 mL×2), dried over Na₂SO₄, filtered and concentrated under reduced pressure to give (S)—N-(5-(5-(3,3-difluoro-2-hydroxypropyl)-1,2,4-oxadiazol-3-yl)-2-methylphenyl)-7-(((1-((2-(trimethylsilyl)ethoxy)methyl)-1H-pyrazol-3-yl)methoxy)methyl)imidazo[1,2-a]pyridine-3-carboxamide (80 mg, crude) as a yellow solid. MS (ESI): m/z for C₃₁H₃₇F₂N₇O₅Si [M+H]+ calcd. 653.26, [MH]⁺ found: 654.4

Step 6: To a solution of (S)—N-(5-(5-(3,3-difluoro-2-hydroxypropyl)-1,2,4-oxadiazol-3-yl)-2-methylphenyl)-7-(((1-((2-(trimethylsilyl)ethoxy)methyl)-1H-pyrazol-3-yl)methoxy)methyl)imidazo[1,2-a]pyridine-3-carboxamide (80 mg, 1.0 eq) in DCM (0.9 mL) was added TFA (0.3 mL). The mixture was stirred at 25° C. for 1 hour. The reaction mixture was concentrated under reduced pressure. The residue was purified by prep-HPLC (FA condition; column: C18 150×30 mm; mobile phase: [water(FA)-ACN]; gradient:15%-45% B over 12 min) to give (S)-7-(((1H-pyrazol-5-yl)methoxy)methyl)-N-(5-(5-(3,3-difluoro-2-hydroxypropyl)-1,2,4-oxadiazol-3-yl)-2-methylphenyl)imidazo[1,2-a]pyridine-3-carboxamide (I-586, 13.42 mg) as a yellow solid. MS (ESI): m/z for C₂₅H₂₃F₂N₇O₄[M+H]⁺ calcd. 523.18, [MH]⁺ found: 524.2. ¹H NMR (400 MHz, methanol-d₄) δ=9.46 (d, J=7.2 Hz, 1H), 8.47 (s, 1H), 8.12 (d, J=1.6 Hz, 1H), 7.91 (dd, J=1.6, 8.0 Hz, 1H), 7.72 (s, 1H), 7.61 (d, J=2.0 Hz, 1H), 7.47 (d, J=8.0 Hz, 1H), 7.17 (dd, J=1.6, 7.2 Hz, 1H), 6.40 (d, J=2.4 Hz, 1H), 6.10-5.75 (m, 1H), 4.70 (s, 4H), 4.42-4.25 (m, 1H), 3.30-3.13 (m, 2H), 2.41 (s, 3H).

### Example 69—Preparation of N-[5-[5-[(2S)-3,3-difluoro-2-hydroxy-propyl]-1,2,4-oxadiazol-3-yl]-2-methyl-phenyl]-7-(1H-1,2,4-triazol-3-ylmethoxymethyl)imidazo[1,2-a]pyridine-3-carboxamide (I-587)

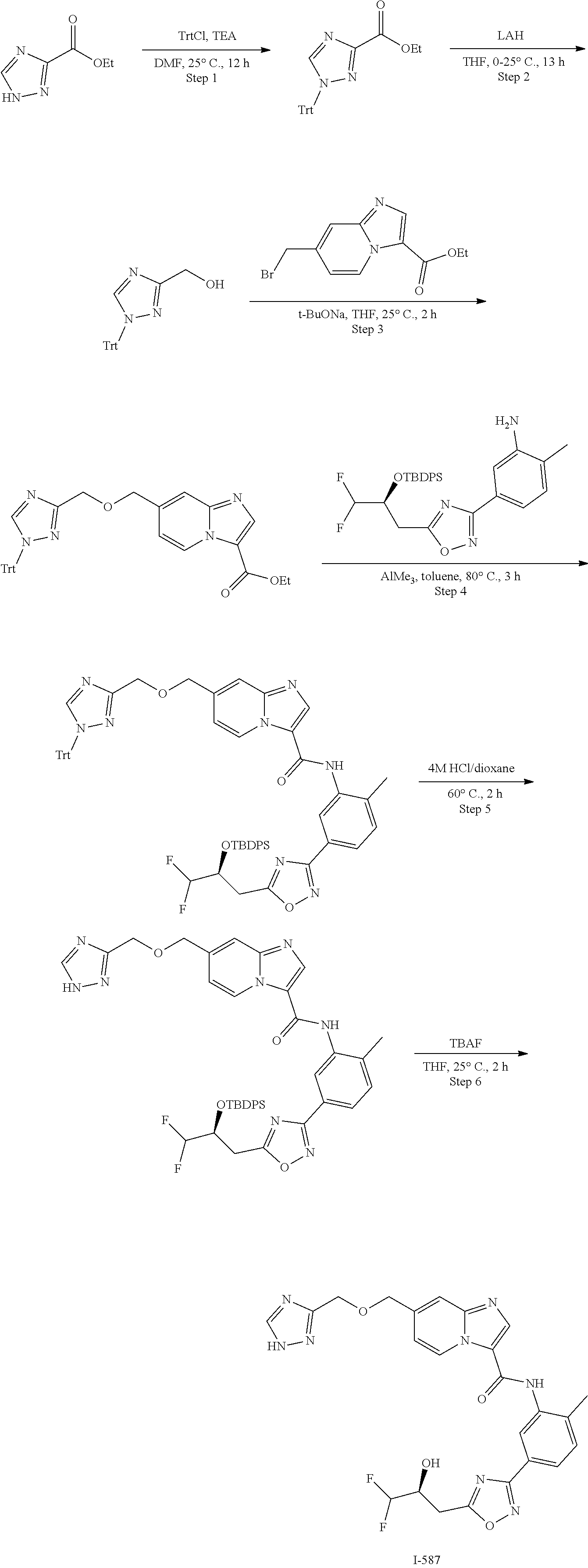

Step 1: A mixture of ethyl 1H-1,2,4-triazole-3-carboxylate (2 g, 1 eq) and triethylamine (1.79 g, 2.47 mL, 1.25 eq) in DMF (25 mL) was stirred at 25° C. for 0.1 h. Then TrtCl (3.95 g, 1 eq) was added and the mixture was stirred at 25° C. for 12 hours. The reaction mixture was partitioned between water (50 mL) and ethyl acetate (200 mL). The organic phase was separated, washed with brine (60 mL×6), dried over Na₂SO₄, filtered and concentrated under reduced pressure to give ethyl 1-trityl-1,2,4-triazole-3-carboxylate (5.1 g, 93.85% yield) as an off-white solid. ¹H NMR (400 MHz, DMSO-d₆) δ 8.36 (s, 1H), 7.43-7.36 (m, 9H), 7.06 (d, J=5.2 Hz, 6H), 4.31 (q, J=7.2 Hz, 2H), 1.28 (t, J=7.2 Hz, 3H)

Step 2: To a solution of ethyl 1-trityl-1,2,4-triazole-3-carboxylate (500 mg, 1.0 eq) in THF (3 mL) was added LAH (2.5 M, 521 μL, 1.0 eq) dropwise. The mixture was stirred at 0° C. for 1 hour under N₂. Then the mixture was allowed to warm to 25° C. for 12 hours. The reaction mixture was cooled to 0° C. and diluted with ethyl acetate (120 mL). Then the mixture was consecutively treated with water (0.5 mL), 15% NaOH solution (0.5 mL), and then water (1.5 mL). The mixture was stirred at 0° C. for 15 minutes. At this time, Na₂SO₄ was added, the mixture was filtered and concentrated under reduced pressure to give (1-trityl-1,2,4-triazol-3-yl)methanol (430 mg, 96.59% yield) as a white solid. ¹H NMR (400 MHz, DMSO-d₆) δ 8.03 (s, 1H), 7.42-7.34 (m, 10H), 7.09-7.03 (m, 6H), 5.30 (t, J=6.4 Hz, 1H), 4.44 (d, J=6.4 Hz, 2H).

Step 3: To a solution of (1-trityl-1,2,4-triazol-3-yl)methanol (200 mg, 1.0 eq) in DMF (3 mL) was added t-BuONa (113 mg, 2.0 eq) at 0° C. The mixture was stirred at 0° C. for 0.5 hours. Then ethyl 7-(bromomethyl)imidazo[1,2-a]pyridine-3-carboxylate (166 mg, 1.0 eq) was added. The mixture was stirred at 25° C. for 1.5 hours. The reaction mixture was diluted with water (50 mL), and extracted with ethyl acetate (50 mL×3). The combined organic layers were washed with brine (30 mL×2), dried over Na₂SO₄, filtered and concentrated in vacuum. The residue was purified by prep-HPLC (column: Waters Xbridge 150*25 mm*5 um; mobile phase: [water(NH₄HCO₃)-ACN]; gradient:45%-75% B over 15 min) to give ethyl 7-[(1-trityl-1,2,4-triazol-3-yl)methoxymethyl]imidazo[1,2-a]pyridine-3-carboxylate (70 mg, 21.98% yield, 100% purity) as colorless oil. ¹H NMR (400 MHz, DMSO-d₆) δ 9.21-9.10 (m, 1H), 8.27 (s, 1H), 8.16 (s, 1H), 7.63 (s, 1H), 7.43-7.31 (m, 9H), 7.14 (dd, J=1.6, 7.2 Hz, 1H), 7.09-7.02 (m, 6H), 4.63 (d, J=8.4 Hz, 4H), 4.36 (q, J=7.2 Hz, 2H), 1.34 (t, J=7.2 Hz, 3H).

Step 4: To a solution of ethyl 7-[(1-trityl-1,2,4-triazol-3-yl)methoxymethyl]imidazo[1,2-a]pyridine-3-carboxylate (65.37 mg, 1.0 eq) in toluene (1 mL) was added AlMe₃ (2 M, 161 μL, 2.5 eq) at 0° C., and the mixture was stirred at 25° C. for 0.5 hour under N₂. Then (S)-5-(5-(2-((tert-butyldiphenylsilyl)oxy)-3,3-difluoropropyl)-1,2,4-oxadiazol-3-yl)-2-methylaniline (70 mg, 1.0 eq) was added and the reaction mixture was stirred at 80° C. for 2.5 hours under N₂. The reaction mixture was quenched with sat. NH₄Cl (1 mL), extracted with DCM (50 mL), washed with brine (20 mL×2), dried over Na₂SO₄, filtered and concentrated in vacuum to give N-[5-[5-[(2S)-2-[tert-butyl(diphenyl)silyl]oxy-3,3-difluoro-propyl]-1,2,4-oxadiazol-3-yl]-2-methyl-phenyl]-7-[(1-trityl-1,2,4-triazol-3-yl)methoxymethyl]imidazo[1,2-a]pyridine-3-carboxamide (110 mg, crude) as yellow oil. ¹H NMR (400 MHz, DMSO-d₆) δ 10.00 (s, 1H), 9.39 (d, J=7.1 Hz, 1H), 8.59 (s, 1H), 8.16 (s, 1H), 7.98 (d, J=1.6 Hz, 1H), 7.70 (dd, J=1.6, 8.0 Hz, 1H), 7.63-7.59 (m, 3H), 7.50-7.45 (m, 3H), 7.43-7.37 (m, 12H), 7.32-7.30 (m, 3H), 7.09-7.05 (m, 7H), 6.27-5.95 (m, 1H), 4.63 (d, J=6.0 Hz, 4H), 4.43-4.33 (m, 1H), 2.37 (s, 3H), 1.28-1.12 (m, 2H), 0.86 (s, 9H).

Step 5: A mixture of N-[5-[5-[(2S)-2-[tert-butyl(diphenyl)silyl]oxy-3,3-difluoro-propyl]-1,2,4-oxadiazol-3-yl]-2-methyl-phenyl]-7-[(1-trityl-1,2,4-triazol-3-yl)methoxymethyl]imidazo[1,2-a]pyridine-3-carboxamide (110 mg, 1 eq) in HCl/dioxane (3 mL) was stirred at 60° C. for 2 hours. The reaction mixture was concentrated in vacuum to give N-[5-[5-[(2S)-2-[tert-butyl(diphenyl)silyl]oxy-3,3-difluoro-propyl]-1,2,4-oxadiazol-3-yl]-2-methyl-phenyl]-7-(1H-1,2,4-triazol-3-ylmethoxymethyl)imidazo[1,2-a]pyridine-3-carboxamide (80 mg, crude) as yellow solid. MS (ESI): m/z for C₄₀H₄₀N₈O₄F₂Si [M+H]⁺ calcd. 763.3 [M+H]⁺ found: 763.3.

Step 6: A mixture of N-[5-[5-[(2S)-2-[tert-butyl(diphenyl)silyl]oxy-3,3-difluoro-propyl]-1,2,4-oxadiazol-3-yl]-2-methyl-phenyl]-7-(1H-1,2,4-triazol-3-ylmethoxymethyl)imidazo[1,2-a]pyridine-3-carboxamide (80 mg, 1 eq) and TBAF (1 M, 210 μL, 2 eq) in THE (1 mL) was stirred at 25° C. for 2 hours. The reaction mixture was diluted with ethyl acetate (50 mL), and washed with brine (20 mL×3). The organic layer was dried over Na₂SO₄, filtered and concentrated in vacuum. The residue was purified by prep-HPLC (column: C18 150×30 mm; mobile phase: [water(FA)-ACN]; gradient:18%-48% B over 7 min) and prep-TLC (SiO₂, methanol:ethyl acetate=1:10) to give N-[5-[5-[(2S)-3,3-difluoro-2-hydroxy-propyl]-1,2,4-oxadiazol-3-yl]-2-methyl-phenyl]-7-(1H-1,2,4-triazol-3-ylmethoxymethyl)imidazo[1,2-a]pyridine-3-carboxamide (I-587, 12.84 mg, 23.35% yield, 100% purity) as white solid. ¹H NMR (400 MHz, DMSO-d₆) δ 14.22-13.93 (m, 1H), 10.01 (s, 1H), 9.41 (d, J=7.2 Hz, 1H), 8.57 (s, 2H), 8.09 (s, 1H), 7.86-7.65 (m, 2H), 7.50 (d, J=8.0 Hz, 1H), 7.20-7.10 (m, 1H), 6.22-5.89 (m, 2H), 4.75-4.59 (m, 4H), 4.30-4.15 (m, 1H), 3.28 (s, 1H), 3.18-3.11 (m, 1H), 2.37 (s, 3H). MS (ESI): m/z for C₂₄H₂₂N₈O₄F₂[M+H]⁺ calcd. 525.1 [M+H]⁺ found: 525.1

### Example 70—Preparation of 7-methoxy-N-(2-methyl-5-(5-((tetrahydro-2H-pyran-2-yl)methyl)-1,2,4-oxadiazol-3-yl)phenyl)imidazo[1,2-a]pyridine-3-carboxamide (I-592)

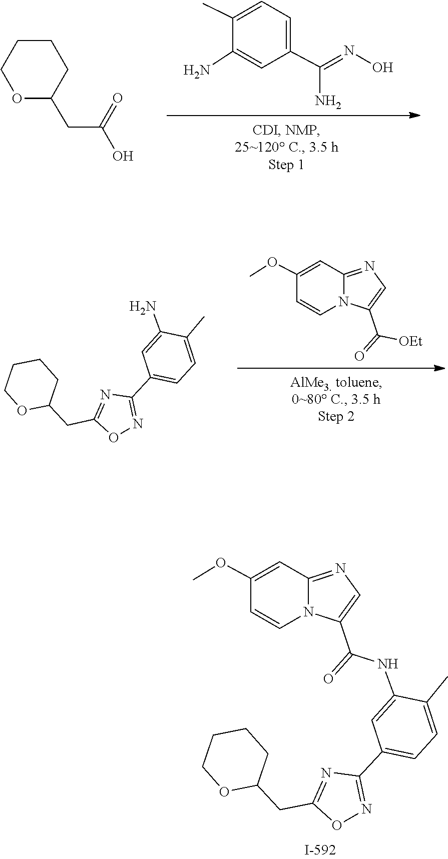

Step 1: To a solution of 2-(tetrahydro-2H-pyran-2-yl)acetic acid(500 mg, 1.0 eq) in NMP (10 mL) was added di(imidazol-1-yl)methanone (675 mg, 1.2 eq). The mixture was stirred at 25° C. for 0.5 hour. The mixture was used without further work up. To the solution was added (Z)-3-amino-N′-hydroxy-4-methylbenzimidamide (570 mg, 1.0 eq). The mixture was stirred at 120° C. for 3 hours. The reaction mixture was diluted with water (50 mL) and extracted with ethyl acetate (80 mL×3). The combined organic layers were washed with brine (40 mL×6), dried over Na₂SO₄, filtered and concentrated under reduced pressure to give 2-methyl-5-(5-((tetrahydro-2H-pyran-2-yl)methyl)-1,2,4-oxadiazol-3-yl) aniline (800 mg, crude) as a yellow solid. ¹H NMR (400 MHz, DMSO-d₆) δ=7.29 (s, 1H), 7.13-7.03 (m, 2H), 5.15 (s, 2H), 3.87-3.72 (m, 2H), 3.32-3.26 (m, 1H), 3.18-3.00 (m, 2H), 2.10 (s, 3H), 1.83-1.67 (m, 2H), 1.57-1.27 (m, 4H). MS (ESI): m/z for C₁₅H₁₉N₃O₂ [M+H]⁺ calcd. 274.15, [M+H]⁺ found: 274.1.

Step 2: To a solution of 2-methyl-5-(5-((tetrahydro-2H-pyran-2-yl)methyl)-1,2,4-oxadiazol-3-yl) aniline (200 mg, 1.0 eq) in the toluene (2 mL) was added AlMe₃ (2 M, 2.5 eq) at 0° C. The mixture was stirred at 25° C. for 0.5 hour under an N₂ atmosphere. Then ethyl 7-methoxyimidazo[1,2-a]pyridine-3-carboxylate (161 mg, 1.0 eq) was added and the mixture was stirred at 80° C. for 3 hours under an N₂ atmosphere. The reaction mixture was quenched by addition of an NH₄Cl solution (2 mL) at 0° C., then was diluted with water 20 mL and extracted with DCM (40 mL×3). The combined organic layers were washed with brine (30 mL×2), dried over Na₂SO₄, filtered and concentrated under reduced pressure to give a residue. The residue was purified by prep-HPLC (basic condition, column: Waters Xbridge 150*25 mm*5 um; mobile phase: [water (ammonia hydroxide v/v)-ACN]; gradient:30%-60% B over 10 min) to give 7-methoxy-N-(2-methyl-5-(5-((tetrahydro-2H-pyran-2-yl)methyl)-1,2,4-oxadiazol-3-yl)phenyl)imidazo[1,2-a]pyridine-3-carboxamide (I-592, 71.28 mg, 22% yield) as a white solid. ¹H NMR (400 MHz, DMSO-d₆) δ=9.89 (s, 1H), 9.27 (d, J=7.6 Hz, 1H), 8.46 (s, 1H), 8.05 (d, J=1.6 Hz, 1H), 7.81-7.77 (m, 1H), 7.48 (d, J=8.0 Hz, 1H), 7.16 (d, J=2.4 Hz, 1H), 6.89-6.85 (m, 1H), 3.90 (s, 3H), 3.86-3.77 (m, 2H), 3.39-3.33 (m, 1H), 3.21-3.06 (m, 2H), 2.35 (s, 3H), 1.83-1.69 (m, 2H), 1.55-1.30 (m, 4H). MS (ESI): m/z for C₂₄H₂₅N₅O₄ [M+H]⁺ calcd. 448.19, [MH]⁺ found: 448.1.

### Example 71—Preparation of 7-(2-(hydroxymethyl)thiazol-4-yl)-N-(2-methyl-5-(5-methyl-1,2,4-oxadiazol-3-yl)phenyl)imidazo[1,2-a]pyridine-3-carboxamide (I-594)

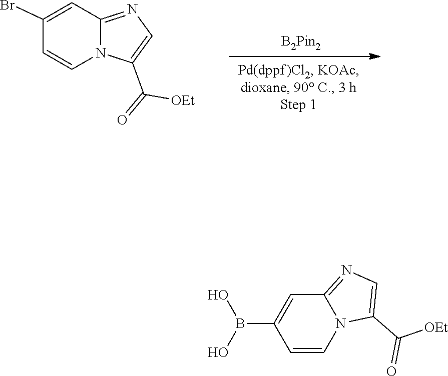 [SMILES: CCOC(=O)c1cnc2cc(Br)ccn12 ; CCOC(=O)c1cnc2cc(B(O)O)ccn12]

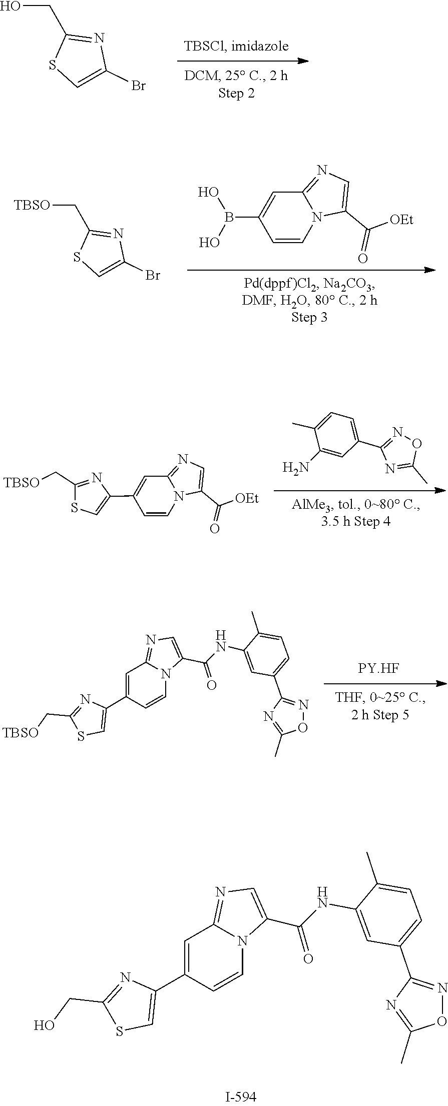

Step 1: To a solution of ethyl 7-bromoimidazo[1,2-a]pyridine-3-carboxylate (5 g, 1.0 eq) and 4,4,5,5-tetramethyl-2-(4,4,5,5-tetramethyl-1,3,2-dioxaborolan-2-yl)-1,3,2-dioxaborolane (9.44 g, 2.0 eq) in dioxane (50 mL) was added potassium acetate (5.47 g, 3.0 eq) and Pd(dppf)Cl₂ (1.36 g, 0.1 eq). The mixture was stirred at 90° C. for 3 hours under an N₂ atmosphere. The reaction was purified directly without work-up. The residue was purified by column chromatography (SiO₂, petroleum ether/ethyl acetate=3/1). The crude product was purified by reversed-phase HPLC (0.1% FA condition) to give (3-(ethoxycarbonyl)imidazo[1,2-a]pyridin-7-yl)boronic acid (130 mg, 464.22 μmol, 2.50% yield, 100% purity, FA salt) as a white solid. ¹H NMR (400 MHz, DMSO-d₆) δ=9.20-9.13 (m, 1H), 8.30 (s, 1H), 8.20 (s, 1H), 7.55-7.45 (m, 1H), 4.40-4.34 (m, 2H), 1.35 (t, J=7.2 Hz, 3H). MS (ESI): m/z for C₁₀H₁₁BN₂O₄[M+H]⁺ calcd. 235.08, [M+H]⁺ found: 235.0

Step 2: To a solution of (4-bromothiazol-2-yl)methanol (500 mg, 1.0 eq) in DCM (5 mL) was added imidazole (263 mg, 1.5 eq) and TBSCI (427 mg, 1.1 eq). The mixture was stirred at 25° C. for 2 hours. The reaction was purified directly without work-up. The residue was purified by column chromatography (SiO₂, petroleum ether/ethyl acetate=19/1) to give 4-bromo-2-(((tert-butyldimethylsilyl)oxy)methyl)thiazole (690 mg, 86% yield) as a colorless oil. ¹H NMR (400 MHz, DMSO-d₆) δ=7.77 (s, 1H), 4.95 (s, 2H), 0.91 (s, 9H), 0.12 (s, 6H). MS (ESI): m/z for C₁₀H₁₈BrNOSSi [M+H]⁺ calcd. 308.01, [M+H]⁺ found: 309.9

Step 3: To a solution of (3-(ethoxycarbonyl)imidazo[1,2-a]pyridin-7-yl)boronic acid (110 mg, 1.0 eq) and 4-bromo-2-(((tert-butyldimethylsilyl)oxy)methyl)thiazole (174 mg, 1.2 eq) in DMF (4 mL) was added Na₂CO₃ (149 mg, 3.0 eq) in H₂O (1 mL) and Pd(dppf)Cl₂ (35 mg, 0.1 eq). The mixture was stirred at 80° C. for 2 hours under an N₂ atmosphere. The reaction mixture was diluted with water (20 mL) and extracted with ethyl acetate (30 mL×3). The combined organic layers were washed with brine (10 mL×6), dried over Na₂SO₄, filtered and concentrated under reduced pressure to give a residue. The residue was purified by column chromatography (SiO₂, petroleum ether/ethyl acetate=4/1) to give ethyl 7-(2-(((tert-butyldimethylsilyl)oxy)methyl)thiazol-4-yl)imidazo[1,2-a]pyridine-3-carboxylate: (170 mg, 84% yield) as a yellow solid. ¹H NMR (400 MHz, DMSO-d₆) δ=9.22 (d, J=7.2 Hz, 1H), 8.43 (s, 1H), 8.32-8.26 (m, 2H), 7.83-7.79 (m, 1H), 5.05 (s, 2H), 4.41-4.34 (m, 2H), 1.35 (t, J=7.2 Hz, 3H), 0.94 (s, 9H), 0.15 (s, 6H). MS (ESI): m/z for C₂₀H₂₇N₃O₃SSi [M+H]⁺ calcd. 418.15, [MH]⁺ found: 418.1.

Step 4: To a solution of ethyl 7-(2-(((tert-butyldimethylsilyl)oxy)methyl)thiazol-4-yl)imidazo[1,2-a]pyridine-3-carboxylate (45 mg, 1.0 eq) in the toluene (1 mL) was added AlMe₃ (2 M, 2.5 eq) at 0° C. The mixture was stirred at 25° C. for 0.5 hour under an N₂ atmosphere. Then ethyl 7-(2-(((tert-butyldimethylsilyl)oxy)methyl)thiazol-4-yl)imidazo[1,2-a]pyridine-3-carboxylate: (100 mg, b1.0 eq) was added, the mixture was stirred at 80° C. for 3 hours under an N₂ atmosphere. The reaction mixture was quenched by the addition of an NH₄Cl solution (1 mL) at 0° C., then was diluted with water (10 mL) and extracted with DCM (20 mL×3). The combined organic layers were washed with brine (15 mL×2), dried over Na₂SO₄, filtered and concentrated under reduced pressure to give 7-(2-(((tert-butyldimethylsilyl)oxy)methyl)thiazol-4-yl)-N-(2-methyl-5-(5-methyl-1,2,4-oxadiazol-3-yl)phenyl)imidazo[1,2-a]pyridine-3-carboxamide (115 mg, crude) as a yellow solid. MS (ESI): m/z for C₂₈H₃₂N₆O₃SSi [M+H]⁺ calcd. 561.20, [MH]⁺ found: 561.2.

Step 5: To a solution of 7-(2-(((tert-butyldimethylsilyl)oxy)methyl)thiazol-4-yl)-N-(2-methyl-5-(5-methyl-1,2,4-oxadiazol-3-yl)phenyl)imidazo[1,2-a]pyridine-3-carboxamide (100 mg, 1.0 eq) in THE (2 mL) was added pyridine-hydrofluoride (1 mL) at 0° C. The mixture was stirred at 25° C. for 2 hours. The reaction mixture was poured into water (2 ml), and then the pH of the solution was adjusted to 7-8 with NaHCO₃ (30 ml), and then extracted with DCM (40 mL×3). The combined organic layers were washed with brine (20 mL×3), dried over Na₂SO₄, filtered and concentrated under reduced pressure to give a residue. The residue was purified by prep-HPLC (FA condition; column: C18 150×30 mm; mobile phase: [water(FA)-ACN]; gradient:25%-55% B over 7 min). The residue was purified by prep-TLC (SiO₂, petroleum ether:ethyl acetate=0:1) to give 7-(2-(hydroxymethyl)thiazol-4-yl)-N-(2-methyl-5-(5-methyl-1,2,4-oxadiazol-3-yl)phenyl)imidazo[1,2-a]pyridine-3-carboxamide (I-594, 4.02 mg, 5.00% yield) as a white solid. ¹H NMR (400 MHz, DMSO-d₆) δ=10.03 (s, 1H), 9.46 (d, J=7.2 Hz, 1H), 8.61 (s, 1H), 8.40 (s, 1H), 8.28 (s, 1H), 8.09 (d, J=1.2 Hz, 1H), 7.83-7.74 (m, 2H), 7.49 (d, J=8.0 Hz, 1H), 6.24-6.15 (m, 1H), 4.84 (d, J=4.8 Hz, 2H), 2.67 (s, 3H), 2.38 (s, 3H). MS (ESI): m/z for C₂₂H₁₈N₆O₃S [M+H]⁺ calcd. 447.12, [MH]⁺ found: 447.1.

### Example 72—Preparation of 7-(5-(hydroxymethyl)thiazol-4-yl)-N-(2-methyl-5-(5-methyl-1,2,4-oxadiazol-3-yl)phenyl)imidazo[1,2-a]pyridine-3-carboxamide (I-595)

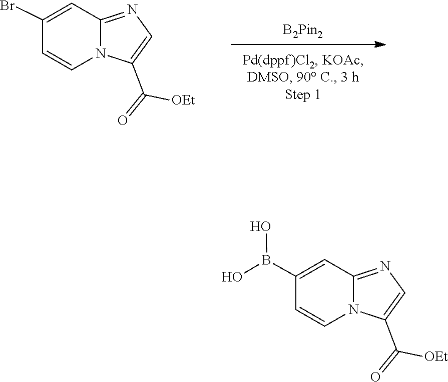 [SMILES: CCOC(=O)c1cnc2cc(Br)ccn12 ; CCOC(=O)c1cnc2cc(B(O)O)ccn12]

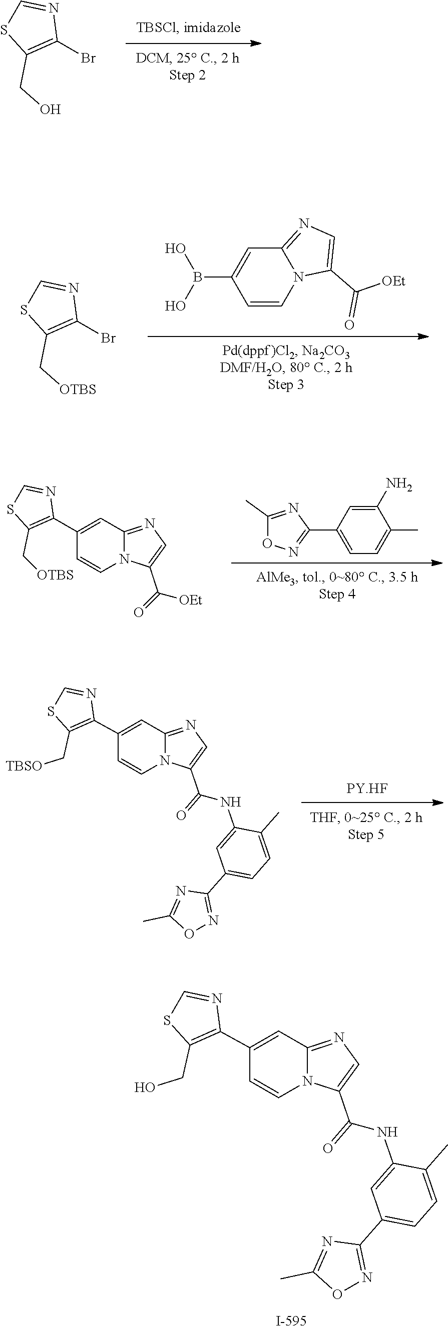

Step 1: To a solution of ethyl 7-bromoimidazo[1,2-a]pyridine-3-carboxylate (2.0 g, 1.0 eq) and 4,4,5,5-tetramethyl-2-(4,4,5,5-tetramethyl-1,3,2-dioxaborolan-2-yl)-1,3,2-dioxaborolane (3.77 g, 2.0 eq) in DMSO (20 mL) was added potassium acetate (2.19 g, 3.0 eq) and Pd(dppf)Cl₂ (544 mg, 0.1 eq). The mixture was stirred at 90° C. for 3 hours under an N₂ atmosphere. The reaction mixture was filtered and concentrated under reduced pressure to give a residue. The crude product was purified by reversed-phase HPLC (0.1% FA condition) to give (3-(ethoxycarbonyl)imidazo[1,2-a]pyridin-7-yl)boronic acid (600 mg, 27% yield) as a white solid. ¹H NMR (400 MHz, DMSO-d₆) δ=8.61 (s, 1H), 7.60 (s, 3H), 4.44-4.27 (m, 2H), 1.42-1.25 (m, 3H). MS (ESI): m/z for C₁₀H₁₁BN₂O₄[M+H]⁺ calcd. 235.08, [M+H]⁺ found: 235.0.

Step 2: To a solution of (4-bromothiazol-5-yl)methanol (500 mg, 1.0 eq) in DCM (5 mL) was added imidazole (263 mg, 1.5 eq) and TBSCI (427 mg, 1.1 eq). The mixture was stirred at 25° C. for 2 hours. The reaction was purified directly without work-up. The residue was purified by column chromatography (SiO₂, petroleum ether/ethyl acetate=19/1) to give 4-bromo-5-(((tert-butyldimethylsilyl)oxy)methyl)thiazole (720 mg, 90% yield) as a colorless oil. ¹H NMR (400 MHz, DMSO-d₆) δ=9.09 (s, 1H), 4.82 (s, 2H), 0.88 (s, 9H), 0.10 (s, 6H). MS (ESI): m/z for C₁₀H₁₈BrNOSSi [M+H]⁺ calcd. 308.01, [M+H]⁺ found: 309.9

Step 3: To a solution of 4-bromo-5-(((tert-butyldimethylsilyl)oxy)methyl)thiazole (200 mg, 1.0 eq) and (3-(ethoxycarbonyl)imidazo[1,2-a]pyridin-7-yl)boronic acid (304 mg, 2.0 eq) in DMF (4 mL) was added Na₂CO₃ (206 mg, 3.0 eq) in H₂O (1 mL) and Pd(dppf)Cl₂ (47 mg, 0.1 eq). The mixture was stirred at 80° C. for 2 hours under an N₂ atmosphere. The reaction mixture was diluted with water (20 mL) and extracted with ethyl acetate (30 mL×3). The combined organic layers were washed with brine (10 mL×6), dried over Na₂SO₄, filtered and concentrated under reduced pressure to give a residue. The residue was purified by column chromatography (SiO₂, petroleum ether/ethyl acetate=4/1) to give ethyl 7-(5-(((tert-butyldimethylsilyl)oxy)methyl)thiazol-4-yl)imidazo[1,2-a]pyridine-3-carboxylate (45 mg, 13% yield) as a yellow solid. MS (ESI): m/z for C₂₀H₂₇N₃O₃SSi [M+H]⁺ calcd. 418.15, [MH]⁺ found: 418.1.

Step 4: To a solution of 2-methyl-5-(5-methyl-1,2,4-oxadiazol-3-yl) aniline (20 mg, 1.0 eq) in the toluene (2 mL) was added AlMe₃ (2 M, 2.5 eq) at 0° C. The mixture was stirred at 25° C. for 0.5 hour under an N₂ atmosphere. Then ethyl 7-(5-(((tert-butyldimethylsilyl)oxy)methyl)thiazol-4-yl)imidazo[1,2-a]pyridine-3-carboxylate (45 mg, 1.0 eq) was added and the mixture was stirred at 80° C. for 3 hours under an N₂ atmosphere. The reaction mixture was quenched by the addition of an NH₄Cl solution (1 mL) at 0° C., then was diluted with water (10 mL) and extracted with DCM (20 mL×3). The combined organic layers were washed with brine (15 mL×2), dried over Na₂SO₄, filtered and concentrated under reduced pressure to give a residue. The residue was purified by prep-TLC (SiO₂, petroleum ether:ethyl acetate=1:2) to give 7-(5-(((tert-butyldimethylsilyl)oxy)methyl)thiazol-4-yl)-N-(2-methyl-5-(5-methyl-1,2,4-oxadiazol-3-yl)phenyl)imidazo[1,2-a]pyridine-3-carboxamide (35 mg, 47% yield) as a yellow solid. MS (ESI): m/z for C₂₈H₃₂N₆O₃SSi [M+H]⁺ calcd. 561.20, [MH]⁺ found. 561.2.

Step 5: To a solution of 7-(5-(((tert-butyldimethylsilyl)oxy)methyl)thiazol-4-yl)-N-(2-methyl-5-(5-methyl-1,2,4-oxadiazol-3-yl)phenyl)imidazo[1,2-a]pyridine-3-carboxamide (35 mg, 1.0 eq) in THE (2 mL) was added pyridine-hydrofluoride (1 mL) at 0° C. The mixture was stirred at 25° C. for 2 hours. The reaction mixture was poured into water (2 ml), and then the pH of the solution was adjusted to 6-7 with a NaHCO₃ solution (30 ml), and then extracted with DCM (100 mL×4). The combined organic layers were washed with brine (100 mL×2), dried over Na₂SO₄, filtered and concentrated under reduced pressure to give a residue. The residue was purified by prep-HPLC (FA condition, column: YMC-Actus Triart C18 150*30 mm*7 um; mobile phase: [water(FA)-ACN]; gradient:23%-53% B over 10 min) to give 7-(5-(hydroxymethyl)thiazol-4-yl)-N-(2-methyl-5-(5-methyl-1,2,4-oxadiazol-3-yl)phenyl)imidazo[1,2-a]pyridine-3-carboxamide (I-595, 16.28 mg, 53% yield) as an off-white solid. ¹H NMR (400 MHz, DMSO-d₆) δ=10.05 (s, 1H), 9.50 (d, J=7.2 Hz, 1H), 9.14 (s, 1H), 8.64 (s, 1H), 8.10 (d, J=1.6 Hz, 1H), 8.02 (s, 1H), 7.83-7.78 (m, 1H), 7.52-7.47 (m, 1H), 7.50 (d, J=8.0 Hz, 1H), 6.05 (s, 1H), 4.93 (s, 2H), 2.67 (s, 3H), 2.38 (s, 3H). MS (ESI): m/z for C₂₂H₁₈N₆O₃S [M+H]⁺ calcd. 447.12, [MH]⁺ found: 447.1.

### Example 73—Preparation of (S)-7-(((2H-tetrazol-5-yl)methoxy)methyl)-N-(5-(5-(3,3-difluoro-2-hydroxypropyl)-1,2,4-oxadiazol-3-yl)-2-methylphenyl)imidazo[1,2-a]pyridine-3-carboxamide (I-597)

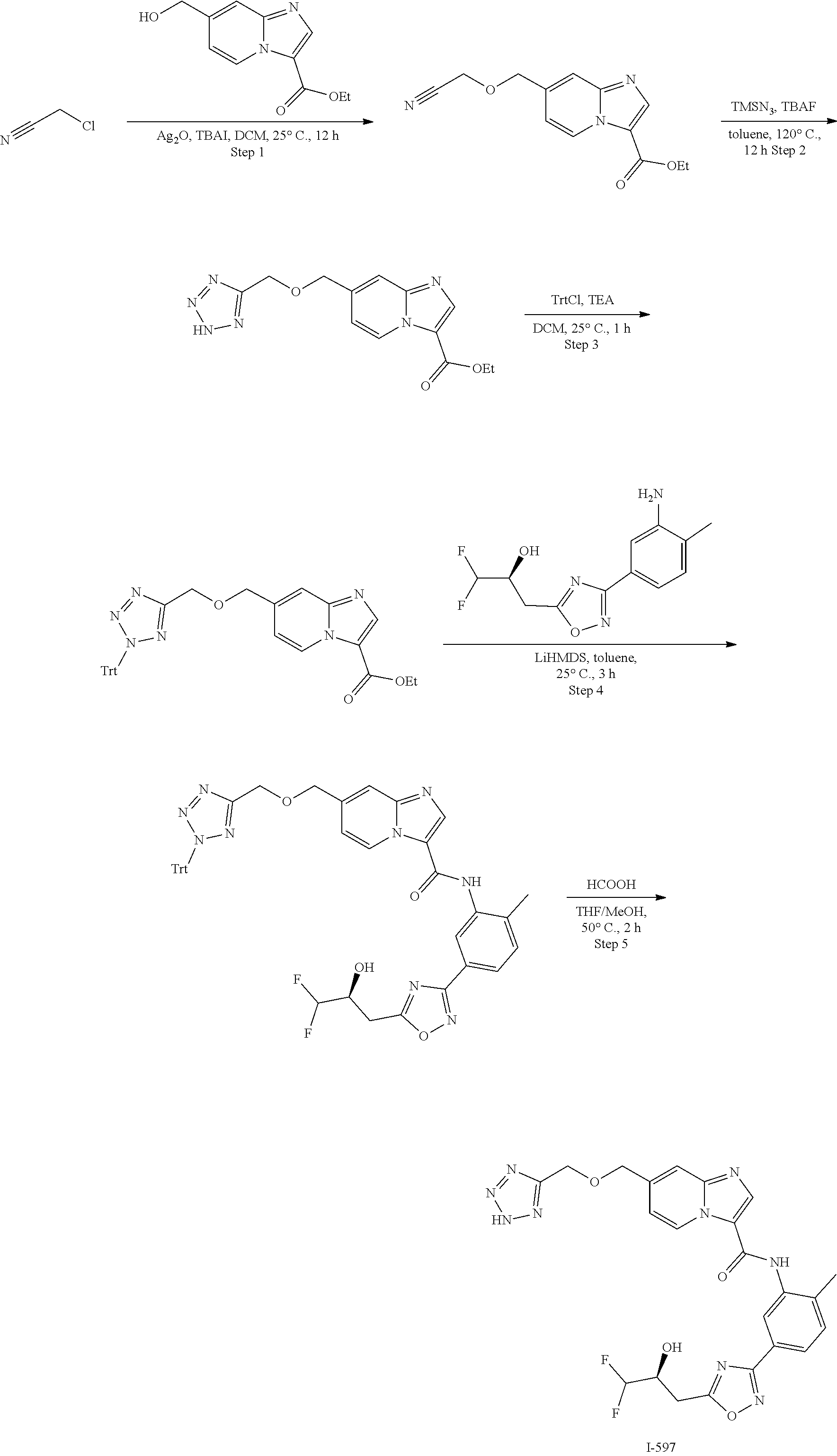

Step 1: To a solution of ethyl 7-(hydroxymethyl)imidazo[1,2-a]pyridine-3-carboxylate (1 g, 1.0 eq) and 2-chloroacetonitrile (342 mg, 1.0 eq) in DCM (50 mL) was added TBAI (167 mg, 0.1 eq) and Ag₂O (10.52 g, 10 eq). The mixture was stirred at 25° C. for 12 hours. The mixture was filtered and concentrated to give a residue. The residue was purified by column chromatography (SiO₂, petroleum ether/ethyl acetate=3/1 to 0/1) to give ethyl 7-((cyanomethoxy)methyl)imidazo[1,2-a]pyridine-3-carboxylate (360 mg, 27% yield) as white solid. ¹H NMR (400 MHz, chloroform-d) δ=9.30 (d, J=7.2 Hz, 1H), 8.31 (s, 1H), 7.70 (d, J=0.8 Hz, 1H), 7.06-6.97 (m, 1H), 4.77 (s, 2H), 4.47-4.40 (m, 2H), 4.37 (s, 2H), 1.43 (t, J=7.2 Hz, 3H). MS (ESI): m/z for C₁₃H₁₃N₃O₃ [M+H]⁺ calcd. 260.27, [M+H]⁺ found: 260.27

Step 2: To a solution of ethyl 7-((cyanomethoxy)methyl)imidazo[1,2-a]pyridine-3-carboxylate (360 mg, 1.0 eq) and TMSN₃ (640 mg, 4.0 eq) in toluene (6 mL) was added TBAF (1 M, 1.39 mL, 1.0 eq). The mixture was stirred at 110° C. for 48 hours. The mixture was concentrated to give a residue. The residue was purified by column chromatography (SiO₂, petroleum ether/ethyl acetate=3/1 to ethyl acetate/methanol=4/1) to give ethyl 7-(((2H-tetrazol-5-yl)methoxy)methyl)imidazo[1,2-a]pyridine-3-carboxylate (0.5 g, crude) as yellow oil. MS (ESI): m/z for C₁₃H₁₄N₆O₃ [M+H]⁺ calcd. 303.3, [M+H]⁺ found: 303.0.

Step 3: To a solution of ethyl 7-(((2H-tetrazol-5-yl)methoxy)methyl)imidazo[1,2-a]pyridine-3-carboxylate (500 mg, 1.0 eq) and triethylamine (502 mg, 3.0 eq) in DCM (6 mL) was added [chloro(diphenyl)methyl]benzene (461 mg 1.0 eq) at 0° C. The mixture was stirred at 25° C. for 1 hour. The mixture was concentrated to give a residue. The residue was purified by column chromatography (SiO₂, petroleum ether/ethyl acetate=3/1 to 1/1) to give ethyl 7-(((2-trityl-2H-tetrazol-5-yl)methoxy)methyl)imidazo[1,2-a]pyridine-3-carboxylate (440 mg, 48% yield) as a white solid. ¹H NMR (400 MHz, chloroform-d) δ=9.22 (d, J=721 Hz, 1H), 8.29 (s, 1H), 7.65 (s, 1H), 7.42-7.30 (m, 9H), 7.16-7.08 (m, 6H), 7.05-6.98 (m, 1H), 4.91 (s, 2H), 4.71 (s, 2H), 4.42 (d, J=7.2 Hz, 2H), 1.43 (t, J=7.2 Hz, 3H). MS (ESI): m/z for C₃₂H₂₈N₆O₃ [M+H]⁺ calcd. 545.62, [M+H]⁺ found: 545.2.

Step 4: To a solution of (S)-3-(3-(3-amino-4-methylphenyl)-1,2,4-oxadiazol-5-yl)-1,1-difluoropropan-2-ol (50 mg, 1.6 eq) and ethyl 7-(((2-trityl-2H-tetrazol-5-yl)methoxy)methyl)imidazo[1,2-a]pyridine-3-carboxylate (63 mg, 1.0 eq) in toluene (2 mL) was added LiHMDS (1 M, 3.0 eq) slowly under nitrogen. The mixture was stirred at 25° C. for 2 hours. The mixture was added to 5 mL of an NH₄Cl solution and extracted with ethyl acetate (30 mL*3). The combined organic layers were washed with brine (10 mL), dried over Na₂SO₄, filtered and concentrated under reduced pressure to give a residue. The residue was purified by column chromatography (SiO₂, petroleum ether/ethyl acetate=1/1) to give (S)—N-(5-(5-(3,3-difluoro-2-hydroxypropyl)-1,2,4-oxadiazol-3-yl)-2-methylphenyl)-7-(((2-trityl-2H-tetrazol-5-yl)methoxy)methyl)imidazo[1,2-a]pyridine-3-carboxamide (60 mg, 57% yield) as white solid. MS (ESI): m/z for C₄₂H₃₅F₂N₉O₄[M+H]⁺ calcd. 768.30, [M+H]⁺ found: 768.4.

Step 5: To a solution of (S)—N-(5-(5-(3,3-difluoro-2-hydroxypropyl)-1,2,4-oxadiazol-3-yl)-2-methylphenyl)-7-(((2-trityl-2H-tetrazol-5-yl)methoxy)methyl)imidazo[1,2-a]pyridine-3-carboxamide (50 mg, 1.0 eq) in THE (2 mL) and MeOH (2 mL) was added HCOOH (250 μL). The mixture was stirred at 50° C. for 1 hour. The mixture was concentrated to give a residue. The residue was purified by prep-HPLC (column: Waters Xbridge 150*25 mm*5 um; mobile phase: [water (ammonia hydroxide v/v)-ACN]; gradient:0%-22% B over 10 min) to give (S)-7-(((2H-tetrazol-5-yl)methoxy)methyl)-N-(5-(5-(3,3-difluoro-2-hydroxypropyl)-1,2,4-oxadiazol-3-yl)-2-methylphenyl)imidazo[1,2-a]pyridine-3-carboxamide (I-597, 12.78 mg, 37% yield) as a yellow solid. ¹H NMR (400 MHz, DMSO-d₆) δ=10.03 (s, 1H), 9.44-9.42 (d, J=8.0 Hz 1H), 8.60 (s, 1H), 8.12 (s, 1H), 7.85-7.83 ((d, J=8.0 Hz, 1H), 7.79 (s, 1H), 7.53-7.52 (d, J=8.0 Hz, 1H), 7.18-7.16 (d, J=8.0 Hz, 1H), 6.25-5.91 (m, 2H), 4.92 (s, 2H), 4.75 (s, 2H), 4.28-4.26 (m, 1H), 3.49-3.47 (m, 1H), 3.20-3.16 (m 1H), 2.39 (s, 3H). MS (ESI): m/z for C₂₃H₂₁F₂N₉O₄[M+H]⁺ calcd. 526.2, [M+H]⁺ found: 526.2.

### Example 74—Preparation of 7-chloro-N-(5-(5-(3,3-difluoroazetidin-1-yl)-1,2,4-oxadiazol-3-yl)-2-methylphenyl)imidazo[1,2-a]pyridine-3-carboxamide (I-601)

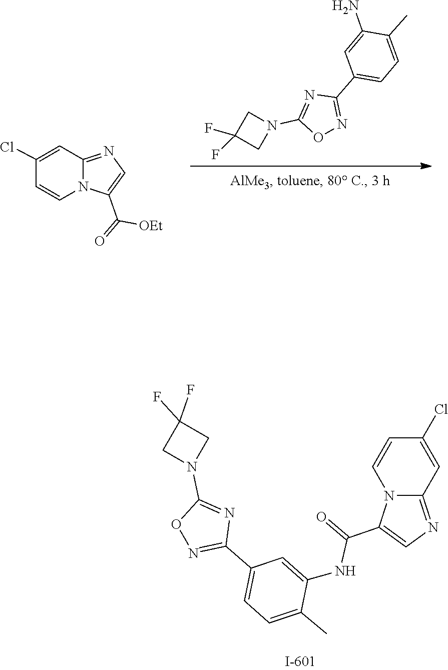 [SMILES: CCOC(=O)c1cnc2cc(Cl)ccn12 ; Cc1ccc(-c2noc(N3CC(F)(F)C3)n2)cc1N ; Cc1ccc(-c2noc(N3CC(F)(F)C3)n2)cc1NC(=O)c1cnc2cc(Cl)ccn12]

Step: To a solution 5-(5-(3,3-difluoroazetidin-1-yl)-1,2,4-oxadiazol-3-yl)-2-methylaniline (40 mg, 1.0 eq) in toluene (1 mL) was added AlMe₃ (2 M, 2.5 eq). The mixture was stirred at 25° C. for 0.5 hour. Then ethyl 7-chloroimidazo[1,2-a]pyridine-3-carboxylate (34 mg, 1.0 eq) was added. The mixture was stirred at 80° C. for 1.5 hours. The reaction mixture was partitioned between NH₄Cl (10 mL) and DCM (20 mL×3). The organic phase was separated, washed with brine (10 mL×3), dried over Na₂SO₄, filtered and concentrated under reduced pressure to give a residue. The residue was purified by prep-HPLC (column: C18 150×30 mm; mobile phase: [water(FA)-ACN]; gradient:45%-75% B over 7 min) to give 7-chloro-N-(5-(5-(3,3-difluoroazetidin-1-yl)-1,2,4-oxadiazol-3-yl)-2-methylphenyl)imidazo[1,2-a]pyridine-3-carboxamide (1-601, 19.19 mg) as a white solid. H NMR (400 MHz, DMSO-d₆) δ=10.14 (s, 1H), 9.47-9.38 (m, 1H), 8.61 (s, 1H), 8.06-7.92 (m, 2H), 7.73 (dd, J=1.6, 8.0 Hz, 1H), 7.46 (d, J=8.0 Hz, 1H), 7.27 (dd, J=2.4, 7.6 Hz, 1H), 4.85-4.70 (m, 4H), 2.35 (s, 3H). MS (ESI): m/z for C₂₀H₁₅ClF₂N₆O₂ [M+H]⁺ calcd. 445.2, [MH]⁺ found: 445.1.

### Example 75—Preparation of S)-7-chloro-N-(5-(5-(2-hydroxypropyl)-1,2,4-oxadiazol-3-yl)-2-methylphenyl)imidazo[1,2-a]pyridine-3-carboxamide (I-602)

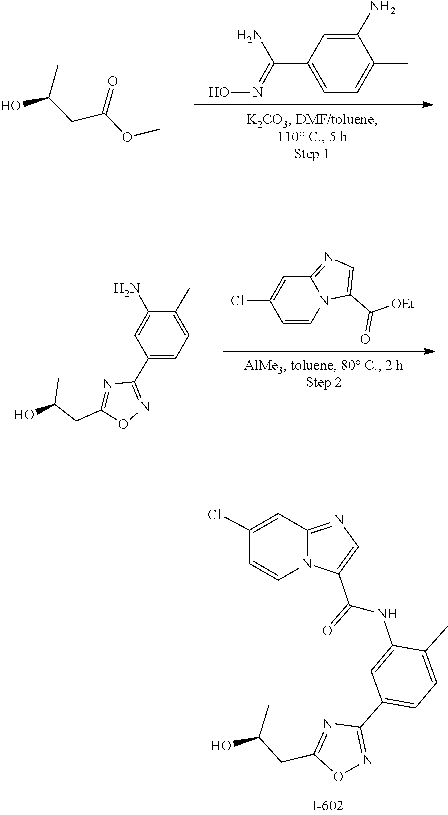

Step 1: To a solution of (Z)-3-amino-N′-hydroxy-4-methylbenzimidamide (699 mg, 1.0 eq) and methyl (S)-3-hydroxybutanoate (500 mg, 1.0 eq) in DMF (2 mL) and toluene (10 mL) was added K₂CO₃ (1.75 g, 3.0 eq). The mixture was stirred at 110° C. for 5 hours. The reaction mixture was partitioned between H₂O (40 mL) and ethyl acetate (50 mL×3). The organic phase was separated, washed with brine (30 mL×3), dried over Na₂SO₄, filtered and concentrated under reduced pressure to give a residue. The crude product was purified by reverse-phase HPLC (0.1% FA condition) to give (S)-1-(3-(3-amino-4-methylphenyl)-1,2,4-oxadiazol-5-yl)propan-2-ol (360 mg) as a white solid. ¹H NMR (400 MHz, DMSO-d₆) δ=7.29 (d, J=1.2 Hz, 1H), 7.19-6.99 (m, 2H), 5.15 (s, 2H), 5.02 (d, J=5.2 Hz, 1H), 4.14 (dd, J=6.0, 7.2 Hz, 1H), 3.07-2.91 (m, 2H), 2.10 (s, 3H), 1.19 (d, J=6.4 Hz, 3H)

Step 2: To a solution of (S)-1-(3-(3-amino-4-methylphenyl)-1,2,4-oxadiazol-5-yl)propan-2-ol (100 mg, 1.0 eq) in toluene (2 mL) was added AlMe₃ (2 M, 2.5 eq). The mixture was stirred at 25° C. for 0.5 hour. Then ethyl 7-chloroimidazo[1,2-a]pyridine-3-carboxylate (96 mg, 1.0 eq) was added. The mixture was stirred at 80° C. for 1.5 hours. The reaction mixture was partitioned between NH₄Cl (10 mL) and DCM (20 mL×3). The organic phase was separated, washed with brine (10 mL×3), dried over Na₂SO₄, filtered and concentrated under reduced pressure to give a residue. The residue was purified by prep-HPLC (column: C18 150×30 mm; mobile phase: [water(FA)-ACN]; gradient:355%-65% B over 7 min) to give S)-7-chloro-N-(5-(5-(2-hydroxypropyl)-1,2,4-oxadiazol-3-yl)-2-methylphenyl)imidazo[1,2-a]pyridine-3-carboxamide (I-602, 55.37 mg) as an off-white solid. ¹H NMR (400 MHz, DMSO-d₆) δ=10.10 (s, 1H), 9.43 (d, J=7.2 Hz, 1H), 8.60 (s, 1H), 8.07 (d, J=1.2 Hz, 1H), 7.99 (d, J=2.0 Hz, 1H), 7.82 (dd, J=1.6, 8.0 Hz, 1H), 7.49 (d, J=8.0 Hz, 1H), 7.26 (dd, J=2.4, 7.2 Hz, 1H), 5.03 (d, J=5.2 Hz, 1H), 4.21-4.12 (m, 1H), 3.13-2.96 (m, 2H), 2.36 (s, 3H), 1.21 (d, J=6.4 Hz, 3H). MS (ESI): m/z for C₂₀H₁₈ClN₅O₃[M+H]⁺ calcd. 412.1, [M+H]⁺ found: 412.1.

### Example 76—Preparation of N-(5-(5-(3,3-difluoroazetidin-1-yl)-1,2,4-oxadiazol-3-yl)-2-methylphenyl)-7-methoxyimidazo[1,2-a]pyridine-3-carboxamide (I-603)

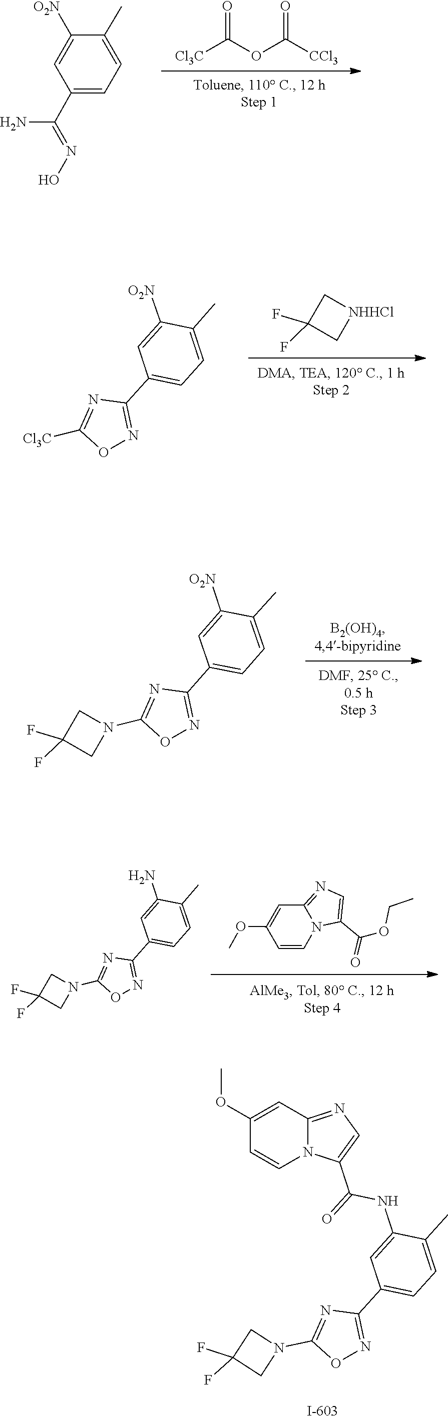

Step 1: A solution of N-hydroxy-4-methyl-3-nitro-benzamidine (2 g, 1.0 eq) and (2,2,2-trichloroacetyl) 2,2,2-trichloroacetate (3.16 g, 1.0 eq) in toluene (20 mL) was heated at 120° C. for 12 hours. The mixture was concentrated to give a residue. The residue was purified by column chromatography (SiO₂, petroleum ether/ethyl acetate=20/1) to give 3-(4-methyl-3-nitrophenyl)-5-(trichloromethyl)-1,2,4-oxadiazole (2.8 g, 80% yield) as a white solid. ¹H NMR (400 MHz, chloroform-d) δ=8.72 (d, J=1.6 Hz, 1H), 8.31-8.21 (m, 1H), 7.53 (d, J=8.0 Hz, 1H), 2.70 (s, 3H).

Step 2: To a solution of 3-(4-methyl-3-nitrophenyl)-5-(trichloromethyl)-1,2,4-oxadiazole (100 mg, 1.0 eq) and 3,3-difluoroazetidine (48 mg, 1.2 eq, HCl) in DMA (1 mL) was added DIEA (120 mg, 3.0 eq). The mixture was stirred at 120° C. for 1 hour. The mixture was added to 20 mL of water and extracted with ethyl acetate (20 mL×3). The combined organic layers were washed with brine (20 mL×3), dried over Na₂SO₄, filtered and concentrated under reduced pressure to give a residue. The residue was purified by column chromatography (SiO₂, petroleum ether/ethyl acetate=20/1 to 10/1) to give 5-(3,3-difluoroazetidin-1-yl)-3-(4-methyl-3-nitrophenyl)-1,2,4-oxadiazole (60 mg, 63% yield) as white solid. ¹H NMR (400 MHz, chloroform-d) δ=8.61 (d, J=1.8 Hz, 1H), 8.16-8.07 (m, 1H), 7.46 (d, J=8.0 Hz, 1H), 4.69 (t, J=11.6 Hz, 4H), 2.68 (s, 3H). MS (ESI): m/z for C₁₂H₁₀F₂N₄O₃[M+H]⁺ calcd. 297.1, [M+H]⁺ found. 297.0

Step 3: To a solution of 5-(3,3-difluoroazetidin-1-yl)-3-(4-methyl-3-nitrophenyl)-1,2,4-oxadiazole (220 mg, 1.0 eq) and 4-(4-pyridyl)pyridine (5.8 mg, 0.05 eq) in DMF (3 mL) was added hypoboric acid (200 mg, 3.0 eq). The mixture was stirred at 25° C. for 10 minutes. The mixture was added to 50 mL of water and white solid was filtered and dried with high vacuum to give 5-(5-(3,3-difluoroazetidin-1-yl)-1,2,4-oxadiazol-3-yl)-2-methylaniline (140 mg, crude) as white solid. ¹H NMR (400 MHz, chloroform-d) δ=7.35-7.31 (m, 1H), 7.31-7.28 (m, 1H), 7.13 (d, J=8.0 Hz, 1H), 4.69 (t, J=11.6 Hz, 4H), 3.71 (s, 2H), 2.21 (s, 3H). MS (ESI): m/z for C₁₂H₁₂F₂N₄O [M+H]⁺ calcd. 267.1, [M+H]⁺ found: 267.0

Step 4: To a solution of 5-(5-(3,3-difluoroazetidin-1-yl)-1,2,4-oxadiazol-3-yl)-2-methylaniline (40 mg, 1.0 eq) in toluene (1 mL) was added AlMe₃ (2 M, 2.5 eq) under N₂. The mixture was stirred at 25° C. for 0.5 hour. To the mixture was added ethyl 7-methoxyimidazo[1,2-a]pyridine-3-carboxylate (33 mg, 1.0 eq). The mixture was stirred at 80° C. for 3 hours. The mixture was cooled to room temperature and added to 10 mL of ice water. It was extracted with DCM (20 mL×3). The combined organic layers were washed with brine (10 mL), dried over Na₂SO₄, filtered and concentrated under reduced pressure to give a residue. The residue was purified by prep-HPLC (column: C18 150×30 mm; mobile phase: [water(FA)-ACN]; gradient:22%-52% B over 7 min) to give N-(5-(5-(3,3-difluoroazetidin-1-yl)-1,2,4-oxadiazol-3-yl)-2-methylphenyl)-7-methoxyimidazo[1,2-a]pyridine-3-carboxamide (I-603, 22.73 mg, 31% yield) as white solid. ¹H NMR (400 MHz, DMSO-d₆) δ=9.88 (s, 1H), 9.26 (d, J=7.6 Hz, 1H), 8.45 (s, 1H), 7.96 (d, J=1.6 Hz, 1H), 7.76-7.64 (m, 1H), 7.44 (d, J=8.0 Hz, 1H), 7.17 (d, J=2.4 Hz, 1H), 6.93-6.77 (m, 1H), 4.75 (t, J=12.4 Hz, 4H), 3.90 (s, 3H), 2.34 (s, 3H). MS (ESI): m/z for C₂₁H₁₈F₂N₆O₃[M+H]+ calcd. 441.1, [M+H]⁺ found: 441.1.

### Example 77—Preparation of 7-Chloro-6-fluoro-N-(5-(5-((1R,2S)-2-fluorocyclopropyl)-1,2,4-oxadiazol-3-yl)-2-methylphenyl)imidazo[1,2-a]pyridine-3-carboxamide (I-613)

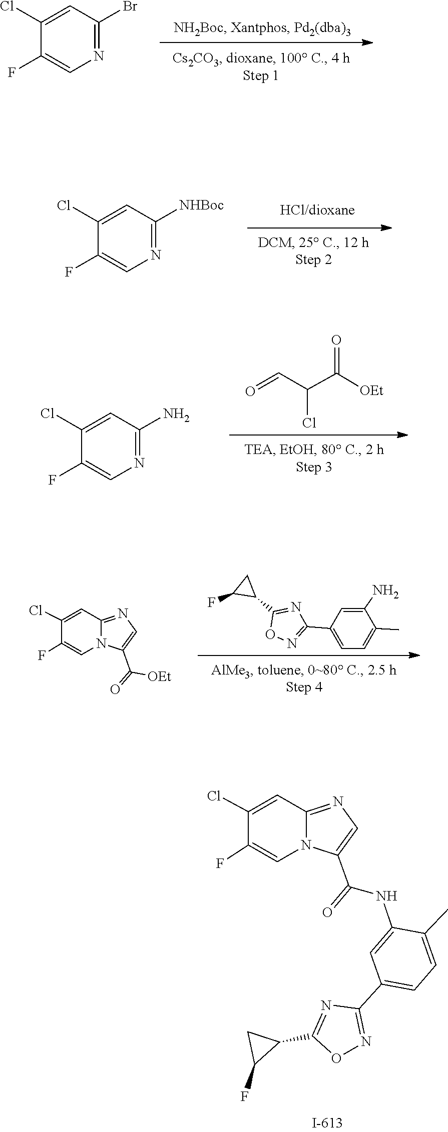

Step 1: To a solution of 2-bromo-4-chloro-5-fluoro-pyridine (3 g, 14.26 mmol, 1.0 eq) and tert-butyl carbamate (1.67 g, 14.26 mmol, 1.0 eq) in dioxane (30 mL) was added cesium carbonate (9.29 g, 28.51 mmol, 2.0 eq), tris(dibenzylideneacetone)dipalladium (1.31 g, 1.43 mmol, 0.1 eq) and (5-diphenylphosphanyl-9,9-dimethylxanthen-4-yl)-diphenylphosphane (1.65 g, 2.85 mmol, 0.2 eq). The mixture was stirred at 100° C. for 4 h under a nitrogen atmosphere. The reaction mixture was diluted with water (80 mL) and extracted with ethyl acetate (120 mL×3). The combined organic layers were washed with a saturated aqueous sodium chloride solution (100 mL×3), dried over anhydrous sodium sulfate, filtered and concentrated under reduced pressure to give a residue. The residue was purified by column chromatography (silica gel, petroleum ether/ethyl acetate=19:1) to give tert-butyl N-(4-chloro-5-fluoro-2-pyridyl)carbamate (2.84 g, 7.68 mmol, 54%) as a white solid. ¹H NMR (400 MHz, DMSO-d₆) δ=10.18 (s, 1H), 8.40 (s, 1H), 7.99 (d, J=5.6 Hz, 1H), 1.47 (s, 9H); MS (ESI): m/z for C₁₀H₁₂ClFN₂O₂[M+H]⁺ calcd. 247.1, [M+H-55]+ found: 190.9.

Step 2: To a solution of tert-butyl N-(4-chloro-5-fluoro-2-pyridyl)carbamate (1.0 g, 4.05 mmol, 1.0 eq) in dichloromethane (5 mL) was added a solution of 2M hydrochloric acid in dioxane (2 M, 5 mL, 2.47 eq). The mixture was stirred at 25° C. for 12 h. The reaction mixture was concentrated under reduced pressure. The crude product was triturated with petroleum ether/ethyl acetate=3:1 at 25° C. for 30 minutes to give 4-chloro-5-fluoro-pyridin-2-amine (550 mg, crude) as a white solid and as a hydrochloride salt. ¹H NMR (400 MHz, DMSO-d₆) δ=8.32 (d, J=3.2 Hz, 1H), 7.15 (d, J=6.0 Hz, 1H), 6.12-5.70 (m, 2H); MS (ESI): m/z for C₅H₄ClFN₂ [M+H]⁺ calcd. 147.0, [M+H]⁺ found:147.0.

Step 3: To a solution of 4-chloro-5-fluoro-pyridin-2-amine (500 mg, 2.73 mmol, 1.0 eq, hydrochloride) in ethanol (5 mL) was added triethylamine (829 mg, 8.20 mmol, 1.14 mL, 3.0 eq) and ethyl 2-chloro-3-oxo-propanoate (823 mg, 5.46 mmol, 2.0 eq). The mixture was stirred at 80° C. for 2 h. The reaction mixture was concentrated to remove the solvent under reduced pressure. The residue was purified by column chromatography (silica gel, petroleum ether/ethyl acetate=9/1). The crude product was purified by reversed-phase HPLC (0.1% formic acid condition) to give ethyl 7-chloro-6-fluoro-imidazo[1,2-a]pyridine-3-carboxylate (150 mg, 0.618 mmol, 23%) as a white solid. ¹H NMR (400 MHz, DMSO-d₆) δ=9.34 (d, J=5.2 Hz, 1H), 8.34 (s, 1H), 8.28 (d, J=7.2 Hz, 1H), 4.42-4.35 (m, 2H), 1.35 (t, J=7.2 Hz, 3H); MS (ESI): m/z for C₁₀H₈ClFN₂O₂ [M+H]⁺ calcd. 243.0, [M+H]⁺ found: 243.0.

Step 4: To a solution of 5-[5-[(1R,2S)-2-fluorocyclopropyl]-1,2,4-oxadiazol-3-yl]-2-methyl-aniline (48 mg, 0.206 mmol, 1.0 eq) in the toluene (1 mL) was added a solution of 2M trimethylaluminum in toluene (2 M, 258 μL, 2.5 eq) at 0° C. The mixture was stirred at 25° C. for 0.5 h under a nitrogen atmosphere. Ethyl 7-chloro-6-fluoro imidazo[1,2-a]pyridine-3-carboxylate (50 mg, 0.206 mmol, 1.0 eq) was added and the mixture was stirred at 80° C. for 2 h under a nitrogen atmosphere. The reaction mixture was quenched by addition of an ammonium chloride solution (1 mL) at 0° C., then diluted with water (10 mL) and extracted with dichloromethane (20 mL×3). The combined organic layers were washed with a saturated aqueous sodium chloride solution (15 mL×2), dried over anhydrous sodium sulfate, filtered and concentrated under reduced pressure to give a residue. The residue was purified by prep-HPLC (formic acid condition; column: C18 150×30 mm; mobile phase: [water (formic acid)—acetonitrile]; gradient:52%-82% B over 7 min) to give 7-chloro-6-fluoro-N-[5-[5-[(1R,2S)-2-fluorocyclopropyl]-1,2,4-oxadiazol-3-yl]-2-methyl-phenyl]imidazo[1,2-a]pyridine-3-carboxamide (I-613, 23 mg, 0.048 mmol, 24%) as a white solid. ¹H NMR (400 MHz, DMSO-d₆) δ=10.17 (s, 1H), 9.59 (d, J=5.2 Hz, 1H), 8.64 (s, 1H), 8.24 (d, J=7.2 Hz, 1H), 8.00 (s, 1H), 7.81-7.76 (m, 1H), 7.49 (d, J=8.0 Hz, 1H), 5.38-5.18 (m, 1H), 3.11-3.00 (m, 1H), 2.35 (s, 3H), 2.01-1.88 (m, 1H), 1.63-1.53 (m, 1H); MS (ESI): m/z for C₂₀H₁₄ClF₂N₅O₂ [M+H]⁺ calcd. 430.1, [M+H]⁺ found: 430.1.

### Example 78—Preparation of 7-Chloro-N-[2-methyl-5-[5-(tetrahydrofuran-2-ylmethyl)-1,2,4-oxadiazol-3-yl]phenyl]imidazo[1,2-a]pyridine-3-carboxamide (I-615)

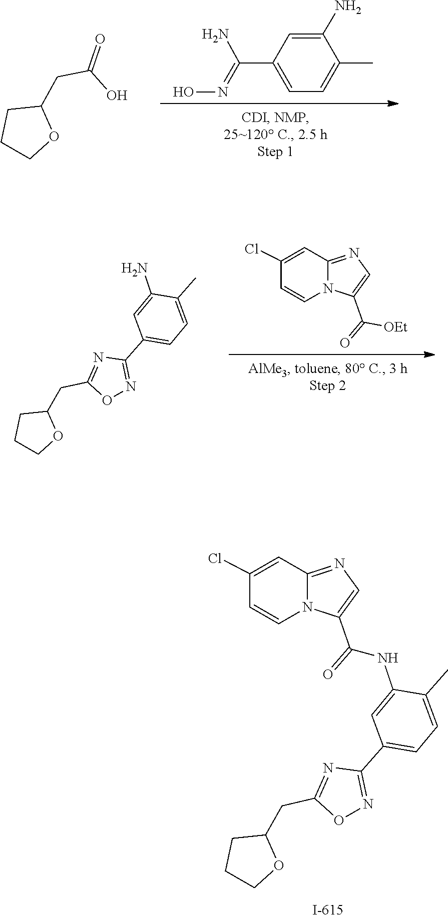 [SMILES: O=C(O)CC1CCCO1 ; Cc1ccc(/C(N)=N/O)cc1N ; Cc1ccc(-c2noc(CC3CCCO3)n2)cc1N ; CCOC(=O)c1cnc2cc(Cl)ccn12 ; Cc1ccc(-c2noc(CC3CCCO3)n2)cc1NC(=O)c1cnc2cc(Cl)ccn12]

Step 1: To a solution of 2-tetrahydrofuran-2-ylacetic acid (500 mg, 3.84 mmol, 1.0 eq) in N-methylpyrrolidine (10 mL) was added carbonyl diimidazole (934 mg, 5.76 mmol, 1.5 eq). The mixture was stirred at 25° C. for 0.5 h. Then 3-amino-N′-hydroxy-4-methyl-benzamidine (635 mg, 3.84 mmol, 1.0 eq) was added to the mixture, and the reaction mixture was stirred at 120° C. for 2 h. The reaction mixture was diluted with water (50 mL), extracted with ethyl acetate (30 mL×3), and the combined organic layers were washed with brine (30 mL×2), dried over anhydrous sodium sulfate, filtered and concentrated under reduced pressure. The residue was purified by column chromatography (silica gel, petroleum ether/ethyl acetate=5:1 to 1:1) to give 2-methyl-5-[5-(tetrahydrofuran-2-ylmethyl)-1,2,4-oxadiazol-3-yl]aniline (500 mg, 1.93 mmol, 50%) as yellow solid. ¹H NMR (400 MHz, DMSO-d₆) δ=7.29 (d, J=1.6 Hz, 1H), 7.13-7.02 (m, 2H), 5.15 (s, 2H), 4.28 (dd, J=5.2, 7.2 Hz, 1H), 3.84-3.72 (m, 1H), 3.69-3.59 (m, 1H), 3.23-3.07 (m, 2H), 2.15-2.02 (m, 4H), 1.92-1.77 (m, 2H), 1.72-1.56 (m, 1H); MS (ESI): m/z for C₁₄H₁₇N₃O₂ [M+H]⁺ calcd.:260.1, [M+H] found: 260.1.

Step 2: To a solution of 2-methyl-5-[5-(tetrahydrofuran-2-ylmethyl)-1,2,4-oxadiazol-3-yl]aniline (50 mg, 0.193 mmol, 1 eq) in toluene (1.0 mL) was added a solution of 2 M trimethylaluminum in toluene (2 M, 241.03 μL, 2.5 eq) at 0° C. The mixture was stirred at 25° C. for 0.5 h under a nitrogen atmosphere. Ethyl 7-chloroimidazo[1,2-a]pyridine-3-carboxylate (43 mg, 0.193 mmol, 1.0 eq) was added, and the reaction mixture was stirred at 80° C. for 3 h under a nitrogen atmosphere. The reaction mixture was quenched by addition of a saturated aqueous ammonium chloride solution (1 mL), extracted with dichloromethane (30 mL), and washed with brine (10 mL×2). The organic layer was dried over anhydrous sodium sulfate, filtered and concentrated in vacuo. The residue was purified by prep-HPLC (column: C18 150×30 mm; mobile phase: [water (formic acid)-acetonitrile]; gradient: 45%-75% B over 7 min) to give 7-chloro-N-[2-methyl-5-[5-(tetrahydrofuran-2-ylmethyl)-1,2,4-oxadiazol-3-yl]phenyl]imidazo [1,2-a]pyridine-3-carboxamide (I-615, 41 mg, 0.084 mmol, 44%) as white solid. ¹H NMR (400 MHz, DMSO-d₆) δ=10.10 (s, 1H), 9.43 (dd, J=0.8, 7.6 Hz, 1H), 8.60 (s, 1H), 8.06 (d, J=1.6 Hz, 1H), 7.98 (dd, J=0.8, 2.4 Hz, 1H), 7.81 (dd, J=1.6, 8.0 Hz, 1H), 7.49 (d, J=8.0 Hz, 1H), 7.26 (dd, J=2.4, 7.6 Hz, 1H), 4.30 (dd, J=5.2, 7.6 Hz, 1H), 3.81-3.71 (m, 1H), 3.63 (m, 1H), 3.26-3.11 (m, 2H), 2.36 (s, 3H), 2.16-2.04 (m, 1H), 1.93-1.78 (m, 2H), 1.73-1.61 (m, 1H); MS (ESI): m/z for C₂₂H₂₀ClN₅O₃[M+H]⁺ calcd: 438.1, [M+H]⁺ found: 438.1.

### Example 79—Preparation of I-7-Chloro-N-(5-(5-(2-hydroxybutyl)-1,2,4-oxadiazol-3-yl)-2-methylphenyl)imidazo[1,2-a]pyridine-3-carboxamide (I-616)

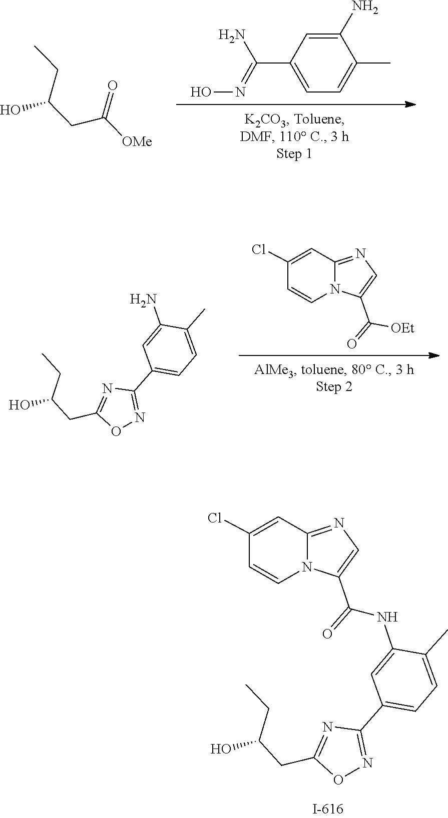

Step 1: To a solution of 3-amin′-N′-hydroxy-4-methyl-benzamidine (300 mg, 1.82 mmol, 1 eq) and methyl (3R)-3-hydroxypentanoate (240 mg, 1.82 mmol, 233 μL, 1.0 eq) in N,N-dimethylformamide (1 mL) and toluene (5 mL) was added potassium carbonate (753 mg, 5.45 mmol, 3.0 eq). The mixture was stirred at 110° C. for 3 h. The reaction mixture was diluted with water (20 mL) and extracted with ethyl acetate (30 mL×3). The combined organic layers were washed with brine (25 mL×2), dried over anhydrous sodium sulfate, filtered and concentrated under reduced pressure to give a residue. The residue was purified by column chromatography (silica gel, petroleum ether/ethyl acetate=10:1 to 1:1) to give (2R)-1-[3-(3-amino-4-methyl-phenyl)-1,2,4-oxadiazol-5-yl]butan-2-ol (260 mg, 1.03 mmol, 57%) as a white solid. MS (ESI): m/z for C₁₃H₁₇N₃O₂. [M+H]⁺ calcd. 248.0, [M+H]⁺ found: 248.2.

Step 2: To a solution of (2R)-1-[3-(3-amino-4-methyl-phenyl)-1,2,4-oxadiazol-5-yl]butan-2-ol (100 mg, 0.404 mmol, 1.0 eq) in toluene (2 mL) was added a solution of 2M trimethylaluminum in toluene (2 M, 505 μL, 2.5 eq) at 25° C. The reaction mixture was stirred at 25° C. for 0.5 h. Ethyl 7-chloroimidazo[1,2-a]pyridine-3-carboxylate (91 mg, 0.404 mmol, 1.0 eq) was added, and the reaction mixture was stirred at 80° C. for 2.5 h. The reaction mixture was quenched with a saturated aqueous ammonium chloride solution (20 mL), then extracted with dichloromethane (25 mL×2). The combined organic layers were washed with brine (25 mL×2), dried over anhydrous sodium sulfate, filtered and concentrated under reduced pressure to give a residue. The residue was purified by prep-HPLC (formic acid condition; column: C18 150×30 mm; mobile phase: [water (formic acid)-acetonitrile]; gradient:388%-68% B over 7 min) to give 7-chloro-N-[5-[5-[(2R)-2-hydroxybutyl]-1,2,4-oxadiazol-3-yl]-2-methyl-phenyl]imidazo[1,2-a]pyridine-3-carboxamide (I-616, 45 mg, 0.106 mmol, 26%) as a white solid. ¹H NMR (400 MHz, DMSO-d₆) δ=10.10 (s, 1H), 9.43 (d, J=7.6 Hz, 1H), 8.60 (s, 1H), 8.07 (d, J=1.6 Hz, 1H), 7.98 (d, J=2.0 Hz, 1H), 7.82 (dd, J=1.6, 8.0 Hz, 1H), 7.-2-7.47 (m, 1H), 7.-9-7.22 (m, 1H), 5.01 (d, J=5.6 Hz, 1H), 3.-8-3.84 (m, 1H), 3.12 (dd, J=4.4, 14.8 Hz, 1H), 3.-2-2.94 (m, 1H), 2.36 (s, 3H), 1.-9-1.43 (m, 2H), 0.92 (t, J=7.2 Hz, 3H); MS (ESI): m/z for C₂₁H₂₀ClN₅O₃ [M+H]⁺ calcd. 426.1, [M+H]⁺ found: 426.1.

### Example 80—Preparation of N-(5-(5-((1R,2S)-2-fluorocyclopropyl)-1,2,4-oxadiazol-3-yl)-2-methylphenyl)-7-(((1-hydroxycyclopropyl)methoxy)methyl)imidazo[1,2-a]pyridine-3-carboxamide (I-622)

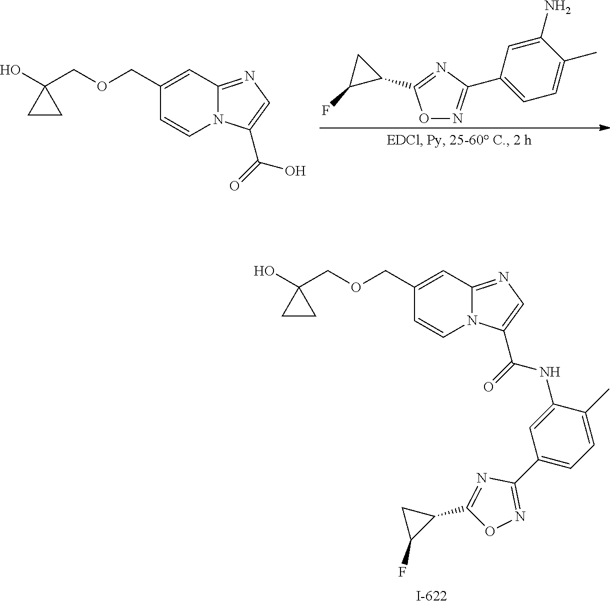 [SMILES: O=C(O)c1cnc2cc(COCC3(O)CC3)ccn12 ; Cc1ccc(-c2noc([C@H]3C[C@@H]3F)n2)cc1N ; Cc1ccc(-c2noc([C@H]3C[C@@H]3F)n2)cc1NC(=O)c1cnc2cc(COCC3(O)CC3)ccn12]

To a solution of 7-[(1-Hydroxycyclopropyl)methoxymethyl]imidazo[1,2-a]pyridine-3-carboxylic acid (45 mg, 0.171 mmol, 1.0 eq) in pyridine (1 mL) was added 3-(ethyliminomethyleneamino)-N,N-dimethyl-propan-1-amine hydrochloride (66 mg, 0.343 mmol, 2.0 eq) and stirred at 25° C. for 0.5 h. 5-[5-[(1R,2S)-2-fluorocyclopropyl]-1,2,4-oxadiazol-3-yl]-2-methyl-aniline (40 mg, 0.171 mmol, 1.0 eq) was added into the mixture and stirred at 60° C. for 1.5 h. The reaction mixture was concentrated under reduced pressure to give a residue. The residue was purified by Prep-HPLC (column: Phenomenex luna C18 150×25 mm×10 um; mobile phase: [water(formic acid)-acetonitrile]; gradient:22%-52% B over 9 min) and Prep-TLC (silica gel, ethyl acetate: methanol=50:1) to give N-[5-[5-[(1R,2S)-2-fluorocyclopropyl]-1,2,4-oxadiazol-3-yl]-2-methyl-phenyl]-7-[(1-hydroxycyclopropyl)methoxymethyl]imidazo[1,2-a]pyridine-3-carboxamide (I-622 23 mg, 0.048 mmol, 27%) as a white solid. ¹H NMR (400 MHz, DMSO-d₆) δ=10.-2-9.99 (m, 1H), 9.-6-9.39 (m, 1H), 8.-9-8.57 (m, 1H), 8.08 (d, J=1.2 Hz, 1H), 7.83 (dd, J=1.6, 8.0 Hz, 1H), 7.71 (s, 1H), 7.54 (d, J=8.0 Hz, 1H), 7.16 (dd, J=1.6, 7.2 Hz, 1H), 5.-8-5.22 (m, 1H), 4.92-4.85 (m, 1H), 4.78 (s, 2H), 3.67 (d, J=5.6 Hz, 2H), 3.-2-3.05 (m, 1H), 2.41 (s, 3H), 2.-8-1.94 (m, 1H), 1.70-1.60 (m, 1H), 0.-4-0.82 (m, 2H), 0.-5-0.64 (m, 2H). MS (ESI): m/z for C₂₅H₂₄FN₅O₄[M+H]⁺ calcd: 478.2 [M+H]⁺ found: 478.2.

### Example 81—Preparation of N-[5-[5-[(2R)-azetidin-2-yl]-1,2,4-oxadiazol-3-yl]-2-methyl-phenyl]-7-(1-methylpyrazol-3-yl)imidazo[1,2-a]pyridine-3-carboxamide (I-624)

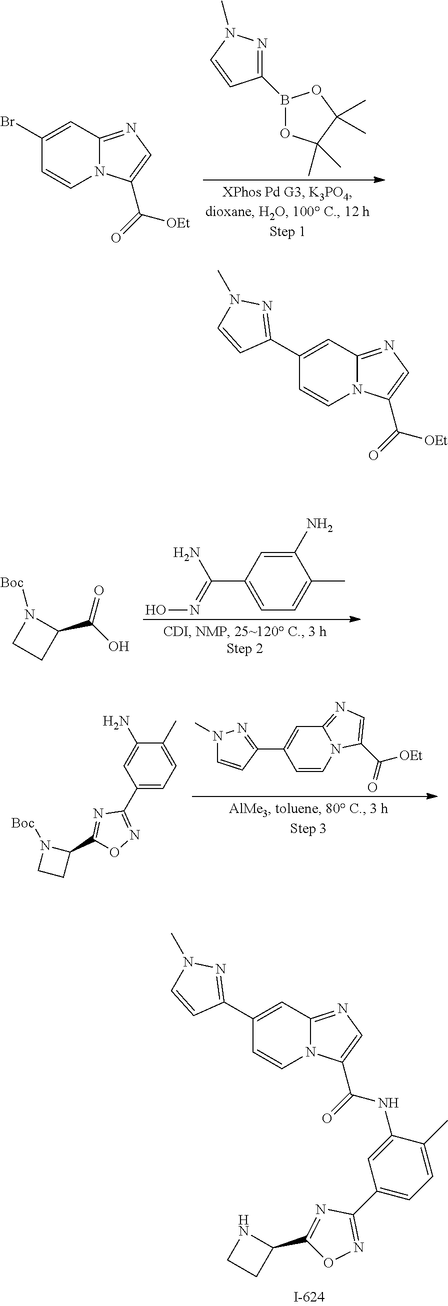

Step 1: To a solution of ethyl 7-bromoimidazo[1,2-a]pyridine-3-carboxylate (1.0 g, 3.72 mmol, 1.0 eq) and 1-methyl-3-(4,4,5,5-tetramethyl-1,3,2-dioxaborolan-2-yl)pyrazole (1.16 g, 5.57 mmol, 1.5 eq) in dioxane (18 mL) and water (3.0 mL) was added potassium phosphate (1.97 g, 9.29 mmol, 2.5 eq) and [2-(2-aminophenyl)phenyl]palladium(1+); dicyclohexyl-[2-(2,4,6-triisopropylphenyl)phenyl]phosphane; methanesulfonate (315 mg, 0.37 mmol, 0.1 eq). The reaction mixture was stirred at 100° C. for 12 h under a nitrogen atmosphere. The reaction mixture was diluted with ethyl acetate (200 mL), washed with brine (100 mL×3), dried over anhydrous sodium sulfate, filtered and concentrated in vacuo. The residue was purified by column chromatography (silica gel, petroleum ether/ethyl acetate=5:1 to 1:3) to give ethyl 7-(1-methylpyrazol-3-yl)imidazo[1,2-a]pyridine-3-carboxylate (1.0 g, 3.70 mmol, 99%) as yellow solid. ¹H NMR (400 MHz, CDCl₃) δ=9.25 (dd, J=0.8, 7.2 Hz, 1H), 8.29 (s, 1H), 8.01 (dd, J=0.8, 1.6 Hz, 1H), 7.60 (dd, J=1.6, 7.2 Hz, 1H), 7.43 (d, J=2.4 Hz, 1H), 6.67 (d, J=2.4 Hz, 1H), 4.42 (m, 2H), 3.98 (s, 3H), 1.42 (t, J=7.2 Hz, 3H); MS (ESI): m/z for C₁₄H₁₄O₂N₄ [M+H]⁺ calcd: 271.1, [M+H]⁺ found: 271.1.

Step 2: To a solution of (2R)-1-tert-butoxycarbonylazetidine-2-carboxylic acid (300 mg, 1.49 mmol, 1.0 eq) in N-Methylpyrrolidone (5.0 mL) was added carbonyl diimidazole (363 mg, 2.24 mmol, 1.5 eq) and the mixture was stirred at 25° C. for 0.5 h. Then to the mixture was added 3-amin′-N′-hydroxy-4-methyl-benzamidine (246 mg, 1.49 mmol, 1.0 eq) and the reaction mixture was stirred at 120° C. for 2.5 h. The reaction mixture was diluted with ethyl acetate (100 mL), washed with brine (50 mL×4), dried over anhydrous sodium sulfate, filtered and concentrated in vacuo. The residue was purified by column chromatography (silica gel, petroleum ether/ethyl acetate=10:1 to 1:1) to give tert-butyl (2R)-2-[3-(3-amino-4-methyl-phenyl)-1,2,4-oxadiazol-5-yl]azetidine-1-carboxylate (380 mg, 1.15 mmol, 77%) as yellow solid. ¹H NMR (400 MHz, CDCl₃) δ=7.-8-7.37 (m, 2H), 7.16 (d, J=7.6 Hz, 1H), 5.42 (dd, J=5.6, 8.8 Hz, 1H), 4.21 (m, 1H), 4.-8-4.01 (m, 1H), 3.75 (s, 2H), 2.-7-2.65 (m, 1H), 2.-0-2.51 (m, 1H), 2.23 (s, 3H), 1.-7-1.28 (m, 9H); MS (ESI): m/z for C₁₇H₂₂N₄O₃ [M+H]⁺ calcd:331.1, [M+Na]⁺ found: 353.1.

Step 3: To a solution of tert-butyl (2R)-2-[3-(3-amino-4-methyl-phenyl)-1,2,4-oxadiazol-5-yl]azetidine-1-carboxylate (100 mg, 0.30 mmol, 1.0 eq) in toluene (3.0 mL) was added a solution of 2 M trimethylaluminum in toluene (2M, 0.38 mL, 2.5 eq) at 0° C., and the mixture was stirred at 25° C. for 0.5 h under a nitrogen atmosphere. Ethyl 7-(1-methylpyrazol-3-yl)imidazo[1,2-a]pyridine-3-carboxylate (82 mg, 0.3 mmol, 1.0 eq) was added, and the reaction mixture was stirred at 80° C. for 3 h under a nitrogen atmosphere. The reaction mixture was quenched by addition of a saturated aqueous ammonium chloride solution (1.0 mL), extracted with dichloromethane (50 mL), washed with brine (10 mL×2), and the organic layer was dried over anhydrous sodium sulfate, filtered and concentrated in vacuo. The residue was purified by prep-HPLC (column: Welch Xtimate C18 150×25 mm×5 um; mobile phase: [water (formic acid)-acetonitrile]; gradient: 1%-30% B over 15 min) and prep-TLC (silica gel, ethyl acetate/methanol=15:1) to give N-[5-[5-[(2R)-azetidin-2-yl]-1,2,4-oxadiazol-3-yl]-2-methyl-phenyl]-7-(1-methylpyrazol-3-yl)imidazo[1,2-a]pyridine-3-carboxamide (I-624, 26 mg, 0.05 mmol, 16%) as a white solid. ¹H NMR (400 MHz, DMSO-d₆) δ=10.01 (s, 1H), 9.43 (d, J=7.6 Hz, 1H), 8.58 (s, 1H), 8.11 (s, 2H), 7.-6-7.80 (m, 2H), 7.-8-7.64 (m, 1H), 7.-3-7.47 (m, 1H), 7.00 (d, J=2.4 Hz, 1H), 5.12 (t, J=7.6 Hz, 1H), 3.93 (s, 3H), 3-9-3.61 (m, 1H), 3.-1-3.37 (m, 2H), 2.-1-2.62 (m, 2H), 2.38 (s, 3H). MS (ESI): m/z for C₂₄H₂₂02N₈ [M+H]⁺ calcd.:455.2 [M+H]⁺ found: 455.2.

### Example 82—Preparation of (S)—N-(5-(5-(5-azaspiro[2.4]heptan-6-yl)-1,2,4-oxadiazol-3-yl)-2-methylphenyl)-7-chloroimidazo[1,2-a]pyridine-3-carboxamide (I-627)

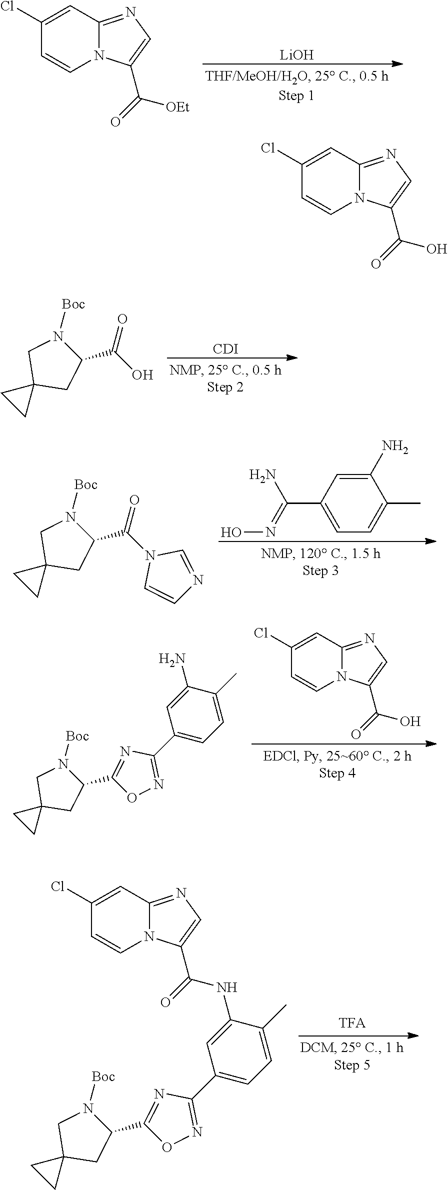

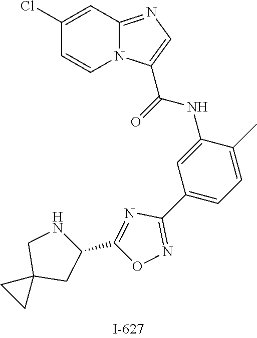 [SMILES: Cc1ccc(-c2noc([C@@H]3CC4(CC4)CN3)n2)cc1NC(=O)c1cnc2cc(Cl)ccn12]

Step 1: To a solution of ethyl 7-chloroimidazo[1,2-a]pyridine-3-carboxylate (1 g, 4.45 mmol, 1.0 eq) in tetrahydrofuran (6 mL), water (6 mL) and methanol (6 mL) was added lithium hydroxide (560 mg, 13.35 mmol, 3.0 eq) and the mixture was stirred at 25° C. for 0.5 h. The mixture was concentrated under reduced pressure to give a residue. The residue was acidified to pH 4 with a solution of 1 M hydrochloric acid. The suspension was filtered to give 7-chloroimidazo[1,2-a]pyridine-3-carboxylic acid (350 mg, crude) as a white solid. MS (ESI): m/z for C₈H₅ClN₂02 [M+H]⁺ calcd:197.1, [M+H]⁺ found:197.0.

Step 2: To a solution of (6S)-5-tert-Butoxycarbonyl-5-azaspiro[2.4]heptane-6-carboxylic acid (775 mg, 3.21 mmol, 1.0 eq) in N-methylpyrrolidone (10 mL) was added carbonyl di-imidazole (624 mg, 3.85 mmol, 1.2 eq). The mixture was stirred at 25° C. for 0.5 h. The solution was used in the next step directly.

Step 3: 3-amin′-N′-hydroxy-4-methyl-benzamidine (531 mg, 3.21 mmol, 1.0 eq) was added into the solution from Step 2 and stirred at 120° C. for 1.5 h. The reaction mixture was diluted with water (30 mL), and extracted with ethyl acetate (50 mL×3). The combined organic layers were washed with brine (30 mL×5), dried over with anhydrous sodium sulfate, filtered and concentrated under reduced pressure to give a residue. The residue was purified by column chromatography (silica gel, petroleum ether:ethyl acetate=1:1 to 1:2) to give tert-butyl (6S)-6-[3-(3-amino-4-methyl-phenyl)-1,2,4-oxadiazol-5-yl]-5-azaspiro[2.4]heptane-5-carboxylate (950 mg, 2.19 mmol, 68%) as a yellow solid. ¹H NMR (400 MHz, DMSO-d₆) δ=7.-5-7.25 (m, 1H), 7.-8-7.03 (m, 2H), 5.17 (s, 2H), 4.03 (s, 2H), 3.39 (s, 1H), 2.10 (s, 3H), 1.19 (s, 6H), 1.-8-1.15 (m, 3H), 0.-0-0.48 (m, 4H). MS (ESI): m/z for C₂₀H₂₆N₄O₃ [M+H]⁺ calcd: 371.2, [M-55]⁺ found: 315.3.

Step 4: To a solution of 7-chloroimidazo[1,2-a]pyridine-3-carboxylic acid (39 mg, 0.198 mmol, 1.0 eq) in pyridine (2 mL) was added 3-(ethyliminomethyleneamino)-N,N-dimethyl-propan-1-amine; hydrochloride (76 mg, 0.396 mmol, 2.0 eq). The mixture was stirred at 25° C. for 0.5 h. Tert-butyl (6S)-6-[3-(3-amino-4-methyl-phenyl)-1,2,4-oxadiazol-5-yl]-5-azaspiro[2.4]heptane-5-carboxylate (81 mg, 0.218 mmol, 1.1 eq) was added into the mixture and stirred at 60° C. for 1.5 h. The reaction mixture was concentrated under reduced pressure to give a residue. The residue was purified by column chromatography (silica gel, petroleum ether:ethyl acetate=1:1 to 0:1) to give tert-butyl (6S)-6-[3-[3-[(7-chloroimidazo[1,2-a]pyridine-3-carbonyl)amino]-4-methyl-phenyl]-1,2,4-oxadiazol-5-yl]-5-azaspiro[2.4]heptane-5-carboxylate (65 mg, 0.114 mmol, 57%) as a white solid. MS (ESI): m/z for C₂₈H₂₉ClN₆O₄[M+H]⁺ calcd: 549.2, [M+H]⁺ found: 549.2.

Step 5: To a solution of tert-butyl (6S)-6-[3-[3-[(7-chloroimidazo[1,2-a]pyridine-3-carbonyl)amino]-4-methyl-phenyl]-1,2,4-oxadiazol-5-yl]-5-azaspiro[2.4]heptane-5-carboxylate (52 mg, 0.095 mmol, 1.0 eq) in dichloromethane (5.4 mL) was added trifluoroacetic acid (1.8 mL). The mixture was stirred at 25° C. for 1 h. The reaction mixture was concentrated under reduced pressure to give a residue. The residue was purified by Prep-HPLC (column: Phenomenex luna C18 150×25 mm×10 um; mobile phase: [water(formic acid)-acetonitrile]; gradient:12%-42% B over 9 min) to give N-[5-[5-[(6S)-5-azaspiro[2.4]heptan-6-yl]-1,2,4-oxadiazol-3-yl]-2-methyl-phenyl]-7-chloro-imidazo[1,2-a]pyridine-3-carboxamide (I-627, 29 mg, 0.063 mmol, 67%) as a white solid. ¹H NMR (400 MHz, DMSO-d₆) δ=10.13 (s, 1H), 9.41 (d, J=7.2 Hz, 1H), 8.61 (s, 1H), 8.18-8.07 (m, 1H), 7.99 (d, J=2.0 Hz, 1H), 7.86 (dd, J=1.6, 8.0 Hz, 1H), 7.54 (d, J=8.0 Hz, 1H), 7.27 (dd, J=2.0, 7.6 Hz, 1H), 5.40-5.29 (m, J=7.6 Hz, 1H), 3.35-3.23 (m, 2H), 2.42 (d, J=8.0 Hz, 2H), 2.37 (s, 3H), 0.80-0.67 (m, 4H). MS (ESI): m/z for C₂₃H₂₁ClN₆O₂ [M+H]⁺ calcd: 449.1, [M+H]⁺ found: 449.1.

### Example 83—Preparation of (S)—N-(5-(5-(5-azaspiro[2.4]heptan-6-yl)-1,2,4-oxadiazol-3-yl)-2-methylphenyl)-7-fluoroimidazo[1,2-a]pyridine-3-carboxamide (I-628)

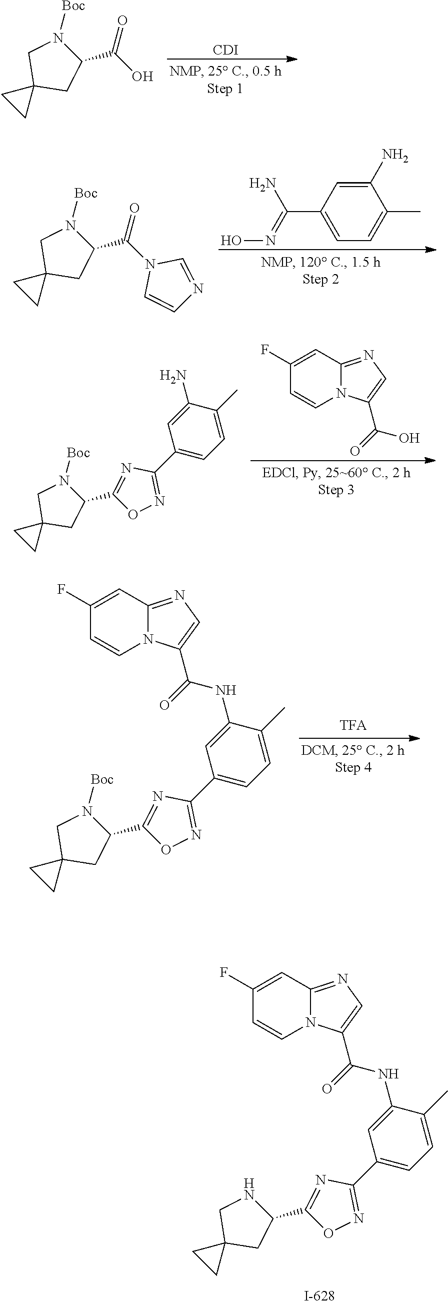

Step 1: To a solution of (6S)-5-tert-Butoxycarbonyl-5-azaspiro[2.4]heptane-6-carboxylic acid (775 mg, 3.21 mmol, 1.0 eq) in N-methylpyrrolidone (10 mL) was added carbonyl di-imidazole (624 mg, 3.85 mmol, 1.2 eq). The mixture was stirred at 25° C. for 0.5 h. The mixture was used in the next step directly.

Step 2: 3-amino-N′-hydroxy-4-methyl-benzamidine (531 mg, 3.21 mmol, 1.0 eq) was added into the mixture from Step 1 and stirred at 120° C. for 1.5 h. The reaction mixture was diluted with water (30 mL), and extracted with ethyl acetate (50 mL×3). The combined organic layers were washed with brine 150 mL (30 mL×5), dried over with anhydrous sodium sulfate, filtered and concentrated under reduced pressure to give a residue. The residue was purified by column chromatography (silica gel, petroleum ether:ethyl acetate=1:1 to 1:2) to give tert-butyl (6S)-6-[3-(3-amino-4-methyl-phenyl)-1,2,4-oxadiazol-5-yl]-5-azaspiro[2.4]heptane-5-carboxylate (950 mg, 2.19 mmol, 68%) as a yellow solid. ¹H NMR (400 MHz, DMSO-d₆) δ=7.35-7.25 (m, 1H), 7.18-7.03 (m, 2H), 5.17 (s, 2H), 4.03 (s, 2H), 3.39 (s, 1H), 2.10 (s, 3H), 1.19 (s, 6H), 1.18-1.15 (m, 3H), 0.70-0.48 (m, 4H). MS (ESI): m/z for C₂₀H₂₆N₄O₃ [M+H]⁺ calcd: 371.2, [M-55]⁺ found: 315.3.

Step 3: To a solution of 7-fluoroimidazo[1,2-a]pyridine-3-carboxylic acid (50 mg, 0.277 mmol, 1.0 eq) in pyridine (2 mL) was added 3-(ethyliminomethyleneamino)-N,N-dimethyl-propan-1-amine hydrochloride (106 mg, 0.555 mmol, 2.0 eq) and the mixture was stirred at 25° C. for 0.5 h. Tert-butyl (6S)-6-[3-(3-amino-4-methyl-phenyl)-1,2,4-oxadiazol-5-yl]-5-azaspiro[2.4]heptane-5-carboxylate (103 mg, 0.277 mmol, 1 eq) was added into the mixture and it was stirred at 60° C. for 1.5 h. The mixture was concentrated under reduced pressure to give a residue. The residue was purified by column chromatography (silica gel, petroleum ether:ethyl acetate=3:1 to 0:1) to give tert-butyl (6S)-6-[3-[3-[(7-fluoroimidazo[1,2-a]pyridine-3-carbonyl)amino]-4-methyl-phenyl]-1,2,4-oxadiazol-5-yl]-5-azaspiro[2.4]heptane-5-carboxylate (100 mg, 0.157 mmol, 57%) as a white solid. ¹H NMR (400 MHz, DMSO-d₆) δ=9.46 (dd, J=6.0, 7.6 Hz, 1H), 8.59 (s, 1H), 8.17-8.06 (m, 1H), 7.86 (dd, J=1.6, 8.0 Hz, 1H), 7.68 (dd, J=2.4, 9.6 Hz, 1H), 7.54 (d, J=8.1 Hz, 1H), 7.30-7.15 (m, 1H), 5.35-5.25 (m, 1H), 3.36-3.18 (m, 2H), 2.42 (d, J=8.0 Hz, 2H), 2.37 (s, 3H), 0.81-0.70 (m, 4H). MS (ESI): m/z for C₂₈H₂₉FN₆O₄ [M+H]⁺ calcd: 533.2, [M-100]⁺ found: 433.2.

Step 4: To a solution of tert-butyl (6S)-6-[3-[3-[(7-fluoroimidazo[1,2-a]pyridine-3-carbonyl)amino]-4-methyl-phenyl]-1,2,4-oxadiazol-5-yl]-5-azaspiro[2.4]heptane-5-carboxylate (95 mg, 0.017 mmol, 1.0 eq) in dichloromethane (5.4 mL) was added trifluoroacetic acid (1.8 mL). The mixture was stirred at 25° C. for 2 h. The reaction mixture was concentrated under reduced pressure to give a residue. The residue was purified by prep-HPLC (column: Phenomenex luna C18 150×25 mm×10 um; mobile phase: [water(formic acid)-acetonitrile]; gradient:8%-38% B over 9 min) to give N-[5-[5-[(6S)-5-azaspiro[2.4]heptan-6-yl]-1,2,4-oxadiazol-3-yl]-2-methyl-phenyl]-7-fluoro-imidazo[1,2-a]pyridine-3-carboxamide (I-628, 46 mg, 0.105 mmol, 59%) as a white solid. ¹H NMR (400 MHz, DMSO-d₆) δ=9.45 (dd, J=6.0, 7.6 Hz, 1H), 8.58 (s, 1H), 8.10 (d, J=1.6 Hz, 1H), 7.85 (dd, J=1.6, 8.0 Hz, 1H), 7.67 (dd, J=2.8, 9.6 Hz, 1H), 7.53 (d, J=8.0 Hz, 1H), 7.24 (dd, J=2.8, 7.6 Hz, 1H), 5.32 (t, J=8.0 Hz, 1H), 3.39-3.19 (m, 2H), 2.42 (d, J=8.0 Hz, 2H), 2.37 (s, 3H), 0.83-0.68 (m, 4H). MS (ESI): m/z for C₂₃H₂₁FN₆O₂[M+H]⁺ calcd: 433.2, [M+H]⁺ found: 433.1.

### Example 84—Preparation of N-(2-methyl-5-(5-methyl-1,2,4-oxadiazol-3-yl)phenyl)-7-(3-(pyrrolidin-1-yl)propoxy)imidazo[1,2-a]pyridine-3-carboxamide (I-629)

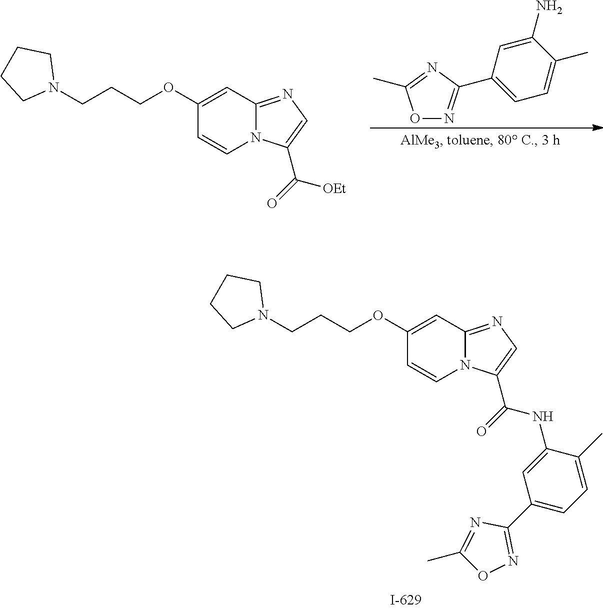

To a solution of 2-methyl-5-(5-methyl-1,2,4-oxadiazol-3-yl) aniline (65 mg, 0.346 mmol, 1.1 eq) in toluene (2 mL) was added a solution of 2M trimethylaluminum in toluene (2 M, 393 μL, 2.5 eq), and the reaction mixture was stirred at 25° C. for 0.5 h. Ethyl 7-(3-pyrrolidin-1-ylpropoxy)imidazo[1,2-a]pyridine-3-carboxylate (100 mg, 0.315 mmol, 1.0 eq) was added and the reaction mixture was stirred at 80° C. for 2.5 h. The reaction mixture was quenched by the addition of a saturated aqueous ammonium chloride solution (20 mL), then was extracted with dichloromethane (25 mL×2). The combined organic layers were washed with brine (25 mL×2), dried over anhydrous sodium sulfate, filtered and concentrated under reduced pressure to give a residue. The residue was purified by prep-TLC (silica gel, petroleum ether/ethyl acetate=0:1) to give N-[2-methyl-5-(5-methyl-1,2,4-oxadiazol-3-yl)phenyl]-7-(3-pyrrolidin-1-ylpropoxy)imidazo[1,2-a]pyridine-3-carboxamide (I-629, 47 mg, 0.102 mmol, 32%) as a white solid. ¹H NMR (400 MHz, DMSO-d₆) δ=9.87 (s, 1H), 9.26 (d, J=7.6 Hz, 1H), 8.45 (s, 1H), 8.06 (d, J=1.6 Hz, 1H), 7.78 (dd, J=1.6, 8.0 Hz, 1H), 7.47 (d, J=8.0 Hz, 1H), 7.14 (d, J=2.4 Hz, 1H), 6.86 (dd, J=2.6, 7.6 Hz, 1H), 4.16 (t, J=6.4 Hz, 2H), 2.66 (s, 3H), 2.57 (t, J=7.2 Hz, 2H), 2.48 (s, 4H), 2.35 (s, 3H), 2.00-1.89 (m, 2H), 1.74-1.63 (m, 4H); MS (ESI): m/z for C₂₅H₂₈N₆O₃ [M+H]⁺ calcd.:461.2, [M+H]⁺ found: 461.2.

### Example 85—Preparation of N-(5-(5-((1R,2S)-2-fluorocyclopropyl)-1,2,4-oxadiazol-3-yl)-2-methylphenyl)-7-(3-(pyrrolidin-1-yl)propoxy)imidazo[1,2-a]pyridine-3-carboxamide (I-630)

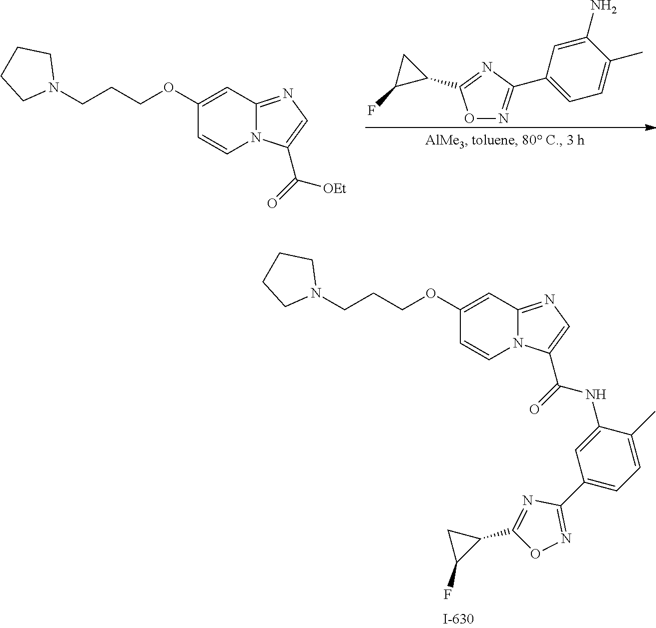 [SMILES: CCOC(=O)c1cnc2cc(OCCCN3CCCC3)ccn12 ; Cc1ccc(-c2noc([C@H]3C[C@@H]3F)n2)cc1N ; Cc1ccc(-c2noc([C@H]3C[C@@H]3F)n2)cc1NC(=O)c1cnc2cc(OCCCN3CCCC3)ccn12]

To a solution of 5-[5-[(1R,2S)-2-fluorocyclopropyl]-1,2,4-oxadiazol-3-yl]-2-methyl-aniline (81 mg, 0.346 mmol, 1.1 eq) in toluene (2 mL) was added a solution of 2M trimethylaluminum in toluene (2 M, 394 μL, 2.5 eq) and the reaction mixture was stirred at 25° C. for 0.5 h. Ethyl 7-(3-pyrrolidin-1-ylpropoxy)imidazo[1,2-a]pyridine-3-carboxylate (100 mg, 0.315 mmol, 1.0 eq) was added, and the reaction mixture was stirred at 80° C. for 2.5 h. The reaction mixture was quenched with a saturated aqueous ammonium chloride solution (20 mL), then extracted with dichloromethane (25 mL×2). The combined organic layers were washed with brine (25 mL×2), dried over anhydrous sodium sulfate, filtered and concentrated under reduced pressure to give a residue. The residue was purified by prep-TLC (silica gel, petroleum ether/ethyl acetate=0:1) to give N-[5-[5-[(1R,2S)-2-fluorocyclopropyl]-1,2,4-oxadiazol-3-yl]-2-methyl-phenyl]-7-(3-pyrrolidin-1-ylpropoxy)imidazo[1,2-a]pyridine-3-carboxamide (I-630, 25 mg, 0.504 mmol, 16%) as a white solid. ¹H NMR (400 MHz, DMSO-d₆) δ=9.89 (s, 1H), 9.25 (d, J=7.6 Hz, 1H), 8.44 (s, 1H), 8.00 (d, J=1.2 Hz, 1H), 7.76 (dd, J=1.6, 8.0 Hz, 1H), 7.47 (d, J=8.0 Hz, 1H), 7.14 (d, J=2.4 Hz, 1H), 6.86 (dd, J=2.4, 7.6 Hz, 1H), 5.39-5.14 (m, 1H), 4.16 (t, J=6.4 Hz, 2H), 3.10-2.99 (m, 1H), 2.58 (d, J=6.8 Hz, 6H), 2.34 (s, 3H), 1.99-1.90 (m, 3H), 1.70 (s, 4H), 1.64-1.53 (m, 1H); MS (ESI): m/z for C₂₇H₂₉FN₆O₃[M+H]⁺ calcd. 505.2, [M+H]⁺ found:505.2.

### Example 86—Preparation of N-(5-(5-(3,3-difluoroazetidin-1-yl)-1,2,4-oxadiazol-3-yl)-2-methylphenyl)-7-(3-(pyrrolidin-1-yl)propoxy)imidazo[1,2-a]pyridine-3-carboxamide (I-631)

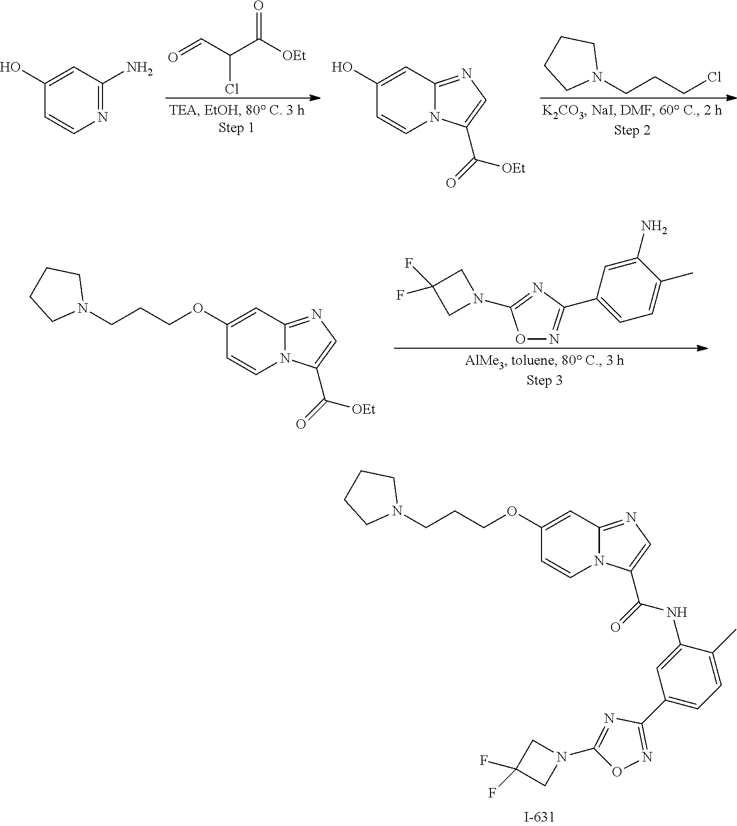

Step 1: To a solution of 2-aminopyridin-4-ol (5 g, 45.41 mmol, 1.0 eq) in ethanol (50 mL) was added triethylamine (14 g, 136.22 mmol, 18.96 mL, 3.0 eq) and ethyl 2-chloro-3-oxo-propanoate (14 g, 90.82 mmol, 2.0 eq). The mixture was stirred at 80° C. for 3 h. The reaction mixture was concentrated under reduced pressure. The residue was diluted with water (100 mL) and extracted with ethyl acetate (50 mL×4). The combined organic layers were washed with brine (40 mL×2), dried over anhydrous sodium sulfate, filtered and concentrated under reduced pressure to give a residue. The residue was purified by column chromatography (silica gel, petroleum ether/ethyl acetate=5:1 to 1:1) to give ethyl 7-hydroxyimidazo[1,2-a]pyridine-3-carboxylate (2 g, 9.57 mmol, 21%) as a white solid. ¹H NMR (400 MHz, DMSO-d₆) δ=9.01 (d, J=7.6 Hz, 1H), 8.09 (s, 1H), 6.93 (d, J=2.4 Hz, 1H), 6.87 (dd, J=2.4, 7.2 Hz, 1H), 4.31 (d, J=7.2 Hz, 2H), 1.32 (t, J=7.2 Hz, 3H); MS (ESI): m/z for C₁₀H₁₀N₂O₃[M+H]⁺ calcd.:207.1, [M+H]⁺ found: 207.1.

Step 2: To a solution of ethyl 7-hydroxyimidazo[1,2-a]pyridine-3-carboxylate (1 g, 4.85 mmol, 1.0 eq) in N,N-dimethylformamide (15 mL) was added potassium carbonate (2.0 g, 14.55 mmol, 3.0 eq), sodium iodide (73 mg, 0.484 mmol, 0.1 eq) and 1-(3-chloropropyl)pyrrolidine (1.43 g, 9.70 mmol, 2.0 eq). The mixture was stirred at 60° C. for 2 h. The reaction mixture was slowly poured into water (50 mL), and then filtered. The filter cake was washed with water (20 mL×2) and petroleum ether (20 mL×2). The cake was collected and concentrated under reduced pressure to give ethyl 7-(3-pyrrolidin-1-ylpropoxy)imidazo[1,2-a]pyridine-3-carboxylate (1.1 g, 3.01 mmol, 62%) as a white solid. ¹H NMR (400 MHz, DMSO-d₆) δ=9.02 (d, J=7.6 Hz, 1H), 8.15 (s, 1H), 7.18 (d, J=2.4 Hz, 1H), 6.93 (dd, J=2.4, 7.6 Hz, 1H), 4.33 (q, J=7.2 Hz, 2H), 4.15 (t, J=6.4 Hz, 2H), 2.56-2.52 (m, 2H), 2.48-2.39 (m, 4H), 1.97-1.88 (m, 2H), 1.72-1.64 (m, 4H), 1.32 (t, J=7.2 Hz, 3H); (ESI): m/z for C₁₇H₂₃N₃O₃ [M+H]⁺ calcd.:318.1, [M+H]⁺ found: 318.1.

Step 3: To a solution of 5-[5-(3,3-difluoroazetidin-1-yl)-1,2,4-oxadiazol-3-yl]-2-methyl-aniline (92 mg, 0.347 mmol, 1.1 eq) in toluene (2 mL) was added a solution of 2M trimethylaluminum in toluene (2 M, 394 μL, 2.5 eq), the reaction mixture was stirred at 25° C. for 0.5 h. Ethyl 7-(3-pyrrolidin-1-ylpropoxy)imidazo[1,2-a]pyridine-3-carboxylate (100 mg, 0.315 mmol, 1.0 eq) was added, and the reaction mixture was stirred at 80° C. for 2.5 h. The reaction mixture was quenched by addition of a saturated aqueous ammonium chloride solution (20 mL), then extracted with dichloromethane (25 mL×2). The combined organic layers were washed with brine (25 mL×2), dried over anhydrous sodium sulfate, filtered and concentrated under reduced pressure to give a residue. The residue was purified by prep-TLC (silica gel, petroleum ether:ethyl acetate=0:1) to give N-[5-[5-(3,3-difluoroazetidin-1-yl)-1,2,4-oxadiazol-3-yl]-2-methyl-phenyl]-7-(3-pyrrolidin-1-ylpropoxy)imidazo[1,2-a]pyridine-3-carboxamide (I-631, 41 mg, 0.077 mmol, 24%) as a white solid. ¹H NMR (400 MHz, DMSO-d₆) δ=9.86 (s, 1H), 9.25 (d, J=7.6 Hz, 1H), 8.44 (s, 1H), 7.95 (d, J=1.6 Hz, 1H), 7.70 (dd, J=1.6, 8.0 Hz, 1H), 7.43 (d, J=8.0 Hz, 1H), 7.14 (d, J=2.4 Hz, 1H), 6.86 (dd, J=2.4, 7.8 Hz, 1H), 4.74 (t, J=12.4 Hz, 4H), 4.16 (t, J=6.4 Hz, 2H), 2.57 (t, J=7.2 Hz, 2H), 2.47 (s, 4H), 2.33 (s, 3H), 1.94 (t, J=6.8 Hz, 2H), 1.73-1.65 (m, 4H); MS (ESI): m/z for C₂₇H₂₉F₂N₇O₃[M+H]⁺ calcd.:538.2, [M+H]⁺ found: 538.2.

### Example 87—Preparation of 7-Methoxy-N-[2-methyl-5-[5-(oxetan-3-ylmethyl)-1,2,4-oxadiazol-3-yl]phenyl]imidazo[1,2-a]pyridine-3-carboxamide (I-633)

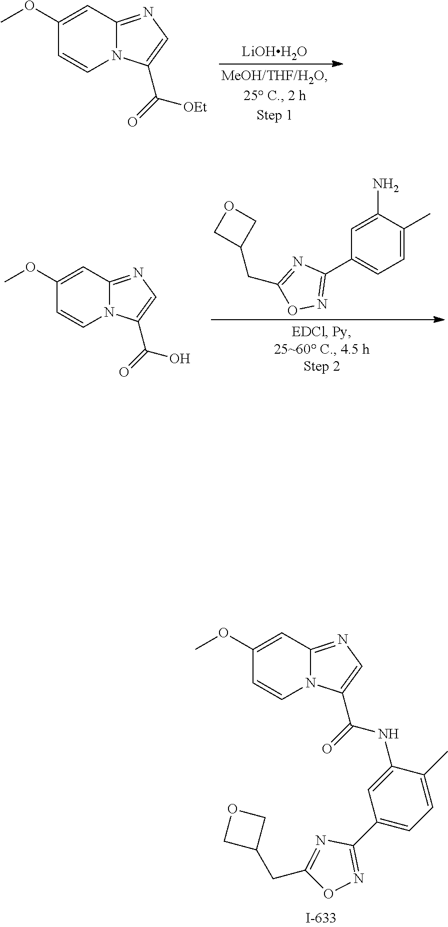

Step 1: To a solution of ethyl 7-methoxyimidazo[1,2-a]pyridine-3-carboxylate (5.00 g, 22.70 mmol, 1.0 eq) in tetrahydrofuran (30 mL) and methanol (10 mL) was added a solution of lithium hydrate monohydrate (1.91 g, 45.41 mmol, 2.0 eq) in water (30 mL). The mixture was stirred at 25° C. for 2 h. The reaction mixture was concentrated under reduced pressure to give a residue. The residue was diluted with water (30 mL) and acidified with citric acid to pH to 3-4. The suspension was filtered and the filter cake was washed with water (20 mL) and dried under vacuum to give 7-methoxyimidazo[1,2-a]pyridine-3-carboxylic acid (3.20 g, 16.64 mmol, 73%) as a white solid. ¹H NMR (400 MHz, DMSO-d₆) δ=9.07 (d, J=7.6 Hz, 1H), 8.10 (s, 1H), 7.17 (d, J=2.4 Hz, 1H), 6.90 (dd, J=2.4, 7.6 Hz, 1H), 3.89 (s, 3H). MS (ESI): m/z for C₉H₈N₂O₃ [M+H]⁺ calcd: 193.1, [M+H]⁺ found: 193.2.

Step 2: A mixture of 7-methoxyimidazo[1,2-a]pyridine-3-carboxylic acid (39 mg, 0.204 mmol, 1.0 eq) and 1-ethyl-(3-(3-dimethylamino)propyl)-carbodiimide hydrochloride (59 mg, 0.306 mmol, 1.5 eq) in pyridine (1 mL) was stirred at 25° C. for 0.5 h. 2-Methyl-5-[5-(oxetan-3-ylmethyl)-1,2,4-oxadiazol-3-yl]aniline (50 mg, 0.204 mmol, 1.0 eq) was added into the mixture and it was stirred at 60° C. for 4 h. The combined reaction mixture was diluted with water (10 mL) and extracted with ethyl acetate (10 mL×3). The combined organic layers were washed with brine (30 mL), dried over with anhydrous sodium sulfate, filtered and concentrated under reduced pressure to give a residue. The residue was purified by Prep-HPLC (formic acid column: YMC-Actus Triart C18 150×30 mm×7 um; mobile phase: [water (formic acid)-acetonitrile]; gradient: 18%-48% B over 10 min) to give 7-methoxy-N-[2-methyl-5-[5-(oxetan-3-ylmethyl)-1,2,4-oxadiazol-3-yl]phenyl]imidazo[1,2-a]pyridine-3-carboxamide (I-633, 25 mg, 0.060 mmol, 15%) as a white solid. ¹H NMR (400 MHz, DMSO-d₆) δ=9.91 (s, 1H), 9.25 (d, J=7.6 Hz, 1H), 8.44 (s, 1H), 8.01 (d, J=1.2 Hz, 1H), 7.77 (dd, J=1.6, 7.6 Hz, 1H), 7.47 (d, J=8.0 Hz, 1H), 7.16 (d, J=2.4 Hz, 1H), 6.87 (dd, J=2.4, 7.6 Hz, 1H), 4.82-4.69 (m, 2H), 4.43 (t, J=6.0 Hz, 2H), 3.89 (s, 3H), 3.48 (dd, J=7.6, 14.4 Hz, 1H), 3.42-3.39 (m, 2H), 2.34 (s, 3H). MS (ESI): m/z for C₂₂H₂₁N₅O₄ [M+H]⁺ calcd: 420.17, [M+H]⁺ found: 420.2.

### Example 88—Preparation of N-[5-[5-[(6S)-5-azaspiro[2.4]heptan-6-yl]-1,2,4-oxadiazol-3-yl]-2-methyl-phenyl]-7-thiazol-4-yl-imidazo[1,2-a]pyridine-3-carboxamide (I-635)

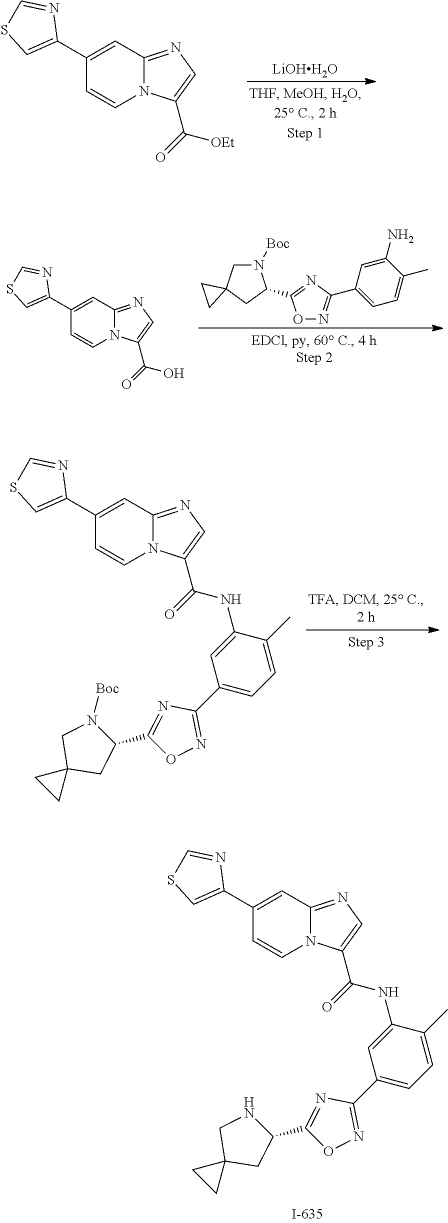 [SMILES: CCOC(=O)c1cnc2cc(-c3cscn3)ccn12 ; "LiOH·H2O" ; O=C(O)c1cnc2cc(-c3cscn3)ccn12 ; Cc1ccc(-c2noc([C@@H]3CC4(CC4)CN3C(=O)OC(C)(C)C)n2)cc1N ; Cc1ccc(-c2noc([C@@H]3CC4(CC4)CN3C(=O)OC(C)(C)C)n2)cc1NC(=O)c1cnc2cc(-c3cscn3)ccn12 ; Cc1ccc(-c2noc([C@@H]3CC4(CC4)CN3)n2)cc1NC(=O)c1cnc2cc(-c3cscn3)ccn12]

Step 1: To a solution of ethyl 7-thiazol-4-ylimidazo[1,2-a]pyridine-3-carboxylate (200 mg, 0.73 mmol, 1.0 eq) in methanol (0.5 mL), tetrahydrofuran (2.5 mL) and water (0.5 mL) was added lithium hydroxide monohydrate (61 mg, 1.46 mmol, 2.0 eq), and the reaction mixture was stirred at 25° C. for 2 h. The reaction mixture was concentrated in vacuo. The residue was diluted with water (50 mL), acidified with 1N hydrochloric acid to pH=5-6. The mixture was filtered, and the filter cake was collected and dried in vacuo to give 7-thiazol-4-ylimidazo[1,2-a]pyridine-3-carboxylic acid (160 mg, 0.65 mmol, 89%) as yellow solid. ¹H NMR (400 MHz, DMSO-d₆) δ=9.33-9.24 (m, 2H), 8.55 (d, J=1.6 Hz, 1H), 8.35 (s, 1H), 8.28 (s, 1H), 7.86 (dd, J=1.6, 7.2 Hz, 1H); MS (ESI): m/z for C₁₁H₇O₂N₃S [M+H]⁺ calcd.:246.1 [M+H]⁺ found: 246.1.

Step 2: To a solution of 7-thiazol-4-ylimidazo[1,2-a]pyridine-3-carboxylic acid (100 mg, 0.41 mmol, 1.0 eq) in pyridine (3.0 mL) was added 3-(ethyliminomethyleneamino)-N,N-dimethyl-propan-1-amine hydrochloride (117.3 mg, 0.61 mmol, 1.5 eq) and the mixture was stirred at 25° C. for 0.5 h. Then tert-butyl (6S)-6-[3-(3-amino-4-methyl-phenyl)-1,2,4-oxadiazol-5-yl]-5-azaspiro[2.4]heptane-5-carboxylate (151.0 mg, 0.41 mmol, 1.0 eq) was added and the reaction mixture was stirred at 60° C. for 3.5 h. The reaction mixture was concentrated in vacuo. The residue was purified by column chromatography (silica gel, petroleum ether/ethyl acetate=5:1 to 0:1) to give tert-butyl (6S)-6-[3-[4-methyl-3-[(7-thiazol-4-ylimidazo[1,2-a]pyridine-3-carbonyl)amino]phenyl]-1,2,4-oxadiazol-5-yl]-5-azaspiro[2.4]heptane-5-carboxylate (40 mg, 0.06 mmol, 16%) as a yellow solid. MS (ESI): m/z for C₃₁H₃₁O₄N₇S [M+H]⁺ calcd.: 598.3 [M+H]⁺ found: 598.3.

Step 3: To a solution of tert-butyl (6S)-6-[3-[4-methyl-3-[(7-thiazol-4-ylimidazo[1,2-a]pyridine-3-carbonyl) amino]phenyl]-1,2,4-oxadiazol-5-yl]-5-azaspiro[2.4]heptane-5-carboxylate (30 mg, 0.05 mmol, 1.0 eq) in dichloromethane (0.6 mL) was added trifluoroacetic acid (461 mg, 4.04 mmol, 80 eq) and the reaction mixture was stirred at 25° C. for 2 h. The reaction mixture was concentrated in vacuo. The residue was purified by prep-HPLC (column: Phenomenex luna C18 150*25 mm*10 um; mobile phase: [water (formic acid)-acetonitrile]; gradient: 8%-38% B over 9 min) to give N-[5-[5-[(6S)-5-azaspiro [2.4]heptan-6-yl]-1,2,4-oxadiazol-3-yl]-2-methyl-phenyl]-7-thiazol-4-yl-imidazo[1,2-a]pyridine-3-carboxamide (I-635, 20 mg, 0.04 mmol, 79%) as an off-white solid. ¹H NMR (400 MHz, DMSO-d₆) δ=10.11 (s, 1H), 9.48 (d, J=7.4 Hz, 1H), 9.31 (d, J=2.0 Hz, 1H), 8.67 (s, 1H), 8.56 (d, J=2.0 Hz, 1H), 8.38 (s, 1H), 8.15 (d, J=1.6 Hz, 1H), 7.86 (m, 2H), 7.55 (d, J=8.0 Hz, 1H), 5.38 (t, J=8.0 Hz, 1H), 3.40-3.36 (m, 1H), 3.31-3.26 (m, 1H), 2.44 (d, J=8.0 Hz, 2H), 2.40 (s, 3H), 0.83-0.71 (m, 4H); MS (ESI): m/z for C₂₆H₂₃02N₇S [M+H]⁺ calcd: 498.1 [M+H]⁺ found: 498.1.

### Example 89—Preparation of N-[5-[5-[(6S)-5-azaspiro[2.4]heptan-6-yl]-1,2,4-oxadiazol-3-yl]-2-methyl-phenyl]-7-(methoxymethyl)imidazo[1,2-a]pyridine-3-carboxamide (I-638)

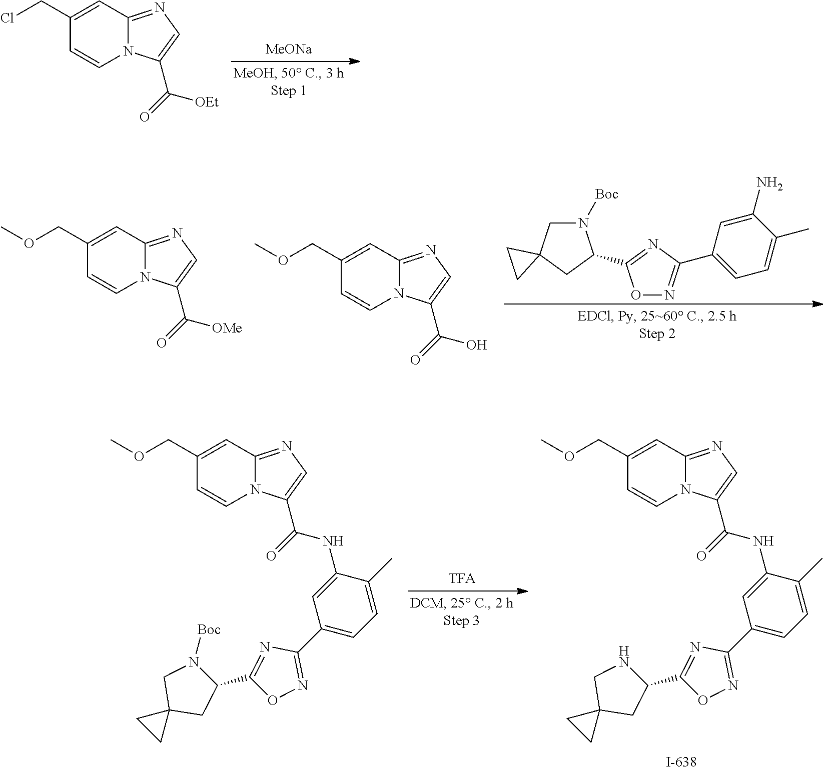

Step 1: To a solution of ethyl 7-(chloromethyl)imidazo[1,2-a]pyridine-3-carboxylate hydrochloride salt (500 mg, 1.82 mmol, 1.0 eq, hydrochloric acid) in methanol (5.0 mL) was added a solution of 5.4 M sodium methanolate in methanol (1.01 mL, 3 eq) and the mixture was stirred at 50° C. for 3 h. The reaction mixture was concentrated in vacuo, diluted with water (50 mL), extracted with ethyl acetate (50 mL), and the organic layer was dried over anhydrous sodium sulfate, filtered and concentrated in vacuo. The residue was purified by column chromatography (silica gel, petroleum ether/ethyl acetate=3:1 to 1:1) to give methyl 7-(methoxymethyl)imidazo[1,2-a]pyridine-3-carboxylate (86 mg, 0.4 mmol, 21%) as yellow solid. The aqueous phase was concentrated in vacuo, the residue was triturated with dichloromethane/methanol=10:1 at 25° C. for 20 min to give 7-(methoxymethyl)imidazo [1,2-a]pyridine-3-carboxylic acid (150 mg, crude) as white solid. ¹H NMR (400 MHz, DMSO-d₆) δ=9.18 (d, J=7.2 Hz, 1H), 8.28 (s, 1H), 7.69 (s, 1H), 7.20 (dd, J=1.6, 7.2 Hz, 1H), 4.55 (s, 2H), 3.88 (s, 3H), 3.36 (s, 3H). MS (ESI): m/z for C₁₁H₁₂03N₂ [M+H]⁺ calcd: 221.1 [M+H]⁺ found: 221.1. ¹H NMR (400 MHz, DMSO-d₆) δ 9.39 (d, J=7.2 Hz, 1H), 8.72 (s, 1H), 7.87 (s, 1H), 7.48 (dd, J=1.2, 7.2 Hz, 1H), 4.66 (s, 2H), 3.40 (s, 3H). MS (ESI): m/z for C₁₀H₁₀O₃N₂[M+H]⁺ calcd: 207.1 [M+H]⁺ found: 207.1.

Step 2: To a solution of 7-(methoxymethyl)imidazo[1,2-a]pyridine-3-carboxylic acid (50 mg, 0.25 mmol, 1 eq) in pyridine (1 mL) was added 3-(ethyliminomethyleneamino)-N,N-dimethyl-propan-1-amine; hydrochloride (69.73 mg, 0.36 mmol, 1.5 eq) and the mixture was stirred at 25° C. for 0.5 h. Then tert-butyl (6S)-6-[3-(3-amino-4-methyl-phenyl)-1,2,4-oxadiazol-5-yl]-5-azaspiro[2.4]heptane-5-carboxylate (80.84 mg, 0.22 mmol, 0.9 eq) was added, and the reaction mixture was stirred at 60° C. for 2 h. The reaction mixture was concentrated in vacuo. The residue was purified by column chromatography (silica gel, petroleum ether/ethyl acetate=5:1 to 0:1) to give tert-butyl (6S)-6-[3-[3-[[7-(methoxymethyl)imidazo[1,2-a]pyridine-3-carbonyl]amino]-4-methyl-phenyl]-1,2,4-oxadiazol-5-yl]-5-azaspiro[2.4]heptane-5-carboxylate (40 mg, 0.07 mmol, 27%) as yellow solid. MS (ESI): m/z for C₃₀H₃₄05N₆ [M+H]⁺ calcd: 559.3, [M+H]⁺ found: 559.3.

Step 3: To a solution of tert-butyl (6S)-6-[3-[3-[[7-(methoxymethyl)imidazo[1,2-a]pyridine-3-carbonyl] amino]-4-methyl-phenyl]-1,2,4-oxadiazol-5-yl]-5-azaspiro[2.4]heptane-5-carboxylate (40 mg, 0.07 mmol, 1.0 eq) in dichloromethane (1.0 mL) was added trifluoroacetic acid (460.50 mg, 4.04 mmol, 0.3 mL, 56 eq) and the reaction mixture was stirred at 25° C. for 2 h. The reaction mixture was concentrated in vacuo. The residue was purified by prep-HPLC (column: Phenomenex luna C18 150*25 mm*10 um; mobile phase: [water (formic acid)-acetonitrile]; gradient:8%-38% B over 9 min) to give N-[5-[5-[(6S)-5-azaspiro[2.4]heptan-6-yl]-1,2,4-oxadiazol-3-yl]-2-methyl-phenyl]-7-(methoxymethyl)imidazo[1,2-a]pyridine-3-carboxamide (I-638, 19 mg, 0.04 mmol, 56%) as yellow solid. ¹H NMR (400 MHz, DMSO-d₆) δ=10.15 (s, 1H), 9.43 (d, J=7.2 Hz, 1H), 8.67 (s, 1H), 8.18-8.10 (m, 1H), 7.86 (dd, J=1.6, 8.0 Hz, 1H), 7.74 (s, 1H), 7.54 (d, J=8.0 Hz, 1H), 7.22 (dd, J=1.6, 7.2 Hz, 1H), 5.37 (t, J=8.0 Hz, 1H), 4.58 (s, 2H), 3.40-3.35 (m, 4H), 3.33-3.25 (m, 1H), 2.44 (d, J=8.0 Hz, 2H), 2.38 (s, 3H), 0.81-0.70 (m, 4H). MS (ESI): m/z for C₂₅H₂₆O₃N₆ [M+H]⁺ calcd: 459.2, [M+H]⁺ found: 459.2.

### Example 90—Preparation of 7-methyl-N-(2-methyl-5-(5-(4,4,4-trifluoro-3-hydroxy-3-methylbutyl)-1,2,4-oxadiazol-3-yl)phenyl)imidazo[1,2-a]pyridine-3-carboxamide (I-752)

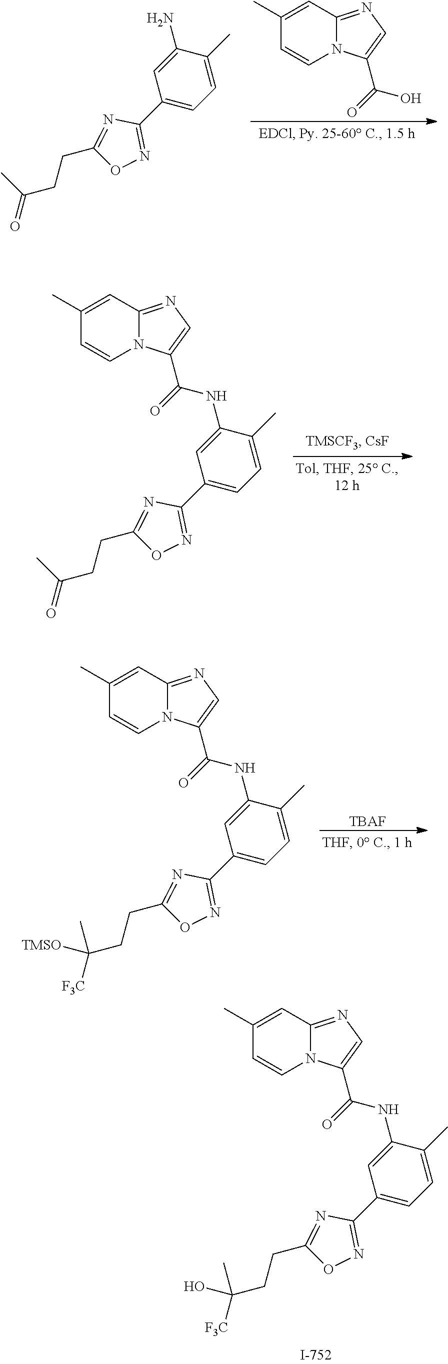

Step 1: To a solution of 7-methylimidazo[1,2-a]pyridine-3-carboxylic acid (500 mg, 1.0 eq) in pyridine (5 mL) was added EDCI (870 mg, 1.6 eq), and the reaction mixture was stirred at 25° C. for 0.5 hour. 4-(3-(3-Amino-4-methylphenyl)-1,2,4-oxadiazol-5-yl)butan-2-one (765 mg, 1.1 eq) was added, then the reaction mixture was stirred at 60° C. for 1 hour. The reaction mixture was diluted with water (50 mL) and extracted with ethyl acetate (50 mL×2). The combined organic layers were washed with brine (30 mL×3), dried over Na₂SO₄, filtered and concentrated under reduced pressure to give a residue. The residue was purified by column chromatography (silica gel, petroleum ether/ethyl acetate=5/1 to 1/1) to give 7-methyl-N-(2-methyl-5-(5-(3-oxobutyl)-1,2,4-oxadiazol-3-yl)phenyl)imidazo[1,2-a]pyridine-3-carboxamide (540 mg) as a white solid. ¹H NMR (400 MHz, METHANOL-d₄) δ=9.36 (d, J=7.2 Hz, 1H), 8.40 (s, 1H), 8.07 (d, J=1.6 Hz, 1H), 7.91-7.82 (m, 1H), 7.50 (d, J=0.8 Hz, 1H), 7.45 (d, J=8.0 Hz, 1H), 7.05-7.00 (m, 1H), 3.20-3.10 (m, 4H), 2.49 (s, 3H), 2.40 (s, 3H), 2.22 (s, 3H).

Step 2: To a solution of 7-methyl-N-(2-methyl-5-(5-(3-oxobutyl)-1,2,4-oxadiazol-3-yl)phenyl)imidazo[1,2-a]pyridine-3-carboxamide (200 mg, 1.0 eq) and CsF (19 mg, 0.25 eq) in toluene (15 mL) and THF (2 mL) was added TMSCF3 (705 mg, 10 eq) dropwise. The mixture was stirred at 25° C. for 12 hours under N₂. The reaction mixture was concentrated in vacuum to give 7-methyl-N-(2-methyl-5-(5-(4,4,4-trifluoro-3-methyl-3-((trimethylsilyl)oxy)butyl)-1,2,4-oxadiazol-3-yl)phenyl)imidazo[1,2-a]pyridine-3-carboxamide (270 mg, crude) as a yellow liquid that was used directly without further purification. MS (ESI): m/z for C₂₆H₃₀F₃N₅O₃Si [M+H]+ calcd.: 546.6 [M+H]+ found: 546.2.

Step 3: To a solution of 7-methyl-N-(2-methyl-5-(5-(4,4,4-trifluoro-3-methyl-3-((trimethylsilyl)oxy)butyl)-1,2,4-oxadiazol-3-yl)phenyl)imidazo[1,2-a]pyridine-3-carboxamide (150 mg, 1.0 eq) in THE (4 mL) was added TBAF (1 M, 1.5 eq) at 0° C. The mixture was stirred at 0° C. for 1 hour. The reaction mixture was diluted with water (30 mL) and extracted with ethyl acetate 120 mL (40 mL×3). The combined organic layers were washed with brine 50 mL (25 mL×2), dried over Na₂SO₄, filtered and concentrated under reduced pressure to give a residue. The residue was purified by column chromatography (silica gel, petroleum ether/ethyl acetate=10/1 to 1/1) to give 7-methyl-N-(2-methyl-5-(5-(4,4,4-trifluoro-3-hydroxy-3-methylbutyl)-1,2,4-oxadiazol-3-yl)phenyl)imidazo[1,2-a]pyridine-3-carboxamide (79 mg) as a yellow solid. ¹H NMR (400 MHz, METHANOL-d₄) δ=9.37 (d, J=7.2 Hz, 1H), 8.41 (s, 1H), 8.10 (d, J=1.6 Hz, 1H), 7.92-7.85 (m, 1H), 7.50 (s, 1H), 7.46 (d, J=8.0 Hz, 1H), 7.05-7.00 (m, 1H), 3.17-3.11 (m, 2H), 2.49 (s, 3H), 2.40 (s, 3H), 2.29 (s, 1H), 2.18 (s, 1H), 1.39 (s, 3H); MS (ESI): m/z for C₂₃H₂₂F₃N₅O₃[M+H]⁺ calcd.: 474.2 [M+H]⁺ found: 474.4.

### Example 91—Preparation of 7-Chloro-N-[2-methyl-5-[5-(oxetan-3-ylmethyl)-1,2,4-oxadiazol-3-yl]phenyl]imidazo[1,2-a]pyridine-3-carboxamide (I-753)

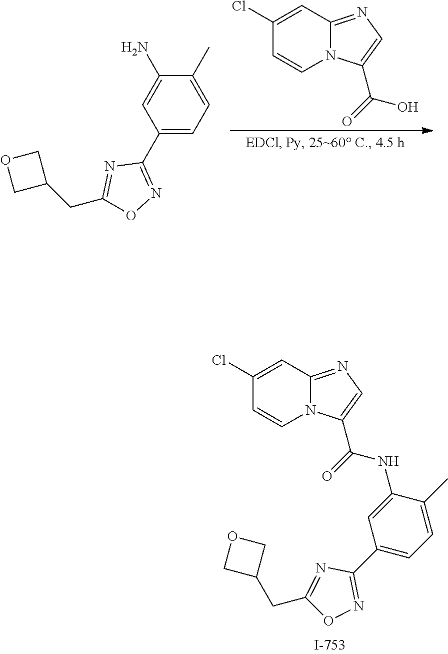 [SMILES: Cc1ccc(-c2noc(CC3COC3)n2)cc1N ; O=C(O)c1cnc2cc(Cl)ccn12 ; Cc1ccc(-c2noc(CC3COC3)n2)cc1NC(=O)c1cnc2cc(Cl)ccn12]

A mixture of 7-chloroimidazo[1,2-a]pyridine-3-carboxylic acid (48 mg, 0.245 mmol, 1.0 eq) and 1-ethyl-(3-(3-dimethylamino)propyl)-carbodiimide hydrochloride (70 mg, 0.367 mmol, 1.5 eq) in pyridine (1 mL) was stirred at 25° C. for 0.5 h. 2-Methyl-5-[5-(oxetan-3-ylmethyl)-1,2,4-oxadiazol-3-yl]aniline (60 mg, 0.245 mmol, 1.0 eq) was added into the mixture and stirred at 60° C. for 4 h. The reaction mixture was diluted with water (10 mL) and extracted with ethyl acetate (10 mL×3). The combined organic layers were washed with brine (30 mL), dried over anhydrous sodium sulfate, filtered, and concentrated under reduced pressure to give a residue. The residue was purified by Prep-HPLC (neutral column: Waters Xbridge 150×25 mm×5 um; mobile phase: [water (ammonium hydrogen carbonate)-acetonitrile]; gradient: 25%-55% B over 15 min) to give 7-chloro-N-[2-methyl-5-[5-(oxetan-3-ylmethyl)-1,2,4-oxadiazol-3-yl]phenyl]imidazo[1,2-a]pyridine-3-carboxamide (6 mg, 0.015 mmol, 6%) as a white solid.

¹H NMR (400 MHz, DMSO-d₆) δ=10.11 (s, 1H), 9.42 (d, J=7.6 Hz, 1H), 8.59 (s, 1H), 8.00 (dd, J=1.6, 14.0 Hz, 2H), 7.79 (s, 1H), 7.49 (d, J=8.0 Hz, 1H), 7.26 (dd, J=2.0, 7.6 Hz, 1H), 4.75 (dd, J=6.0, 7.6 Hz, 2H), 4.44 (t, J=6.0 Hz, 2H), 3.54-3.44 (m, 1H), 3.54-3.44 (m, 1H), 3.43-3.40 (m, 2H), 2.35 (s, 3H); MS (ESI): m/z for C₂₁H₁₈ClN₅O₃[M+H]+ calcd: 424.1, [M+H]+ found: 424.1.

### Example 92—Preparation of N-(2-methyl-5-(1,2,4-oxadiazol-3-yl)phenyl)pyrazolo[1,5-a]pyridine-3-carboxamide (I-640)

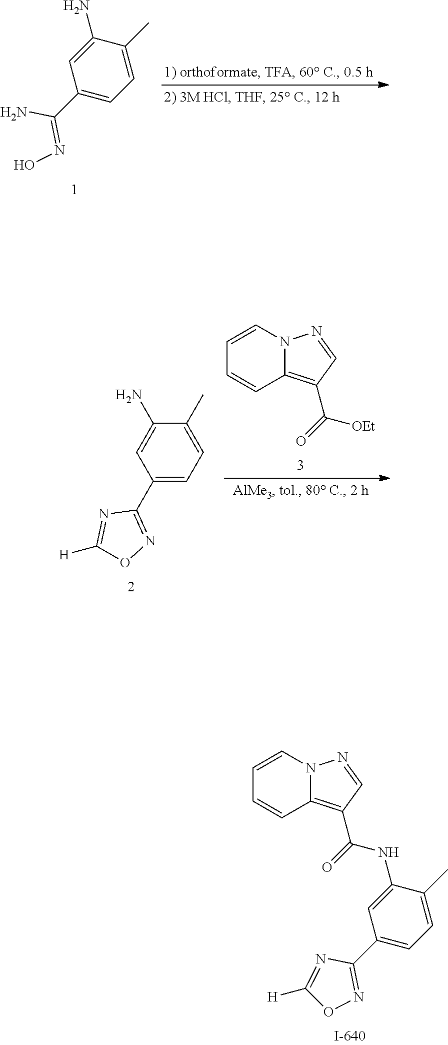

Step 1: To a mixture of 3-amino-N′-hydroxy-4-methyl-benzamidine (1 g, 1.0 eq) and trimethoxymethane (14.52 g, 15.00 mL, 22.60 eq) was added TFA (3.07 g, 26.92 mmol, 2 mL, 4.45 eq). The mixture was stirred at 25° C. for 1 hour and then warmed to to 60° C. The mixture was stirred at 60° C. for 0.5 hour. The mixture was concentrated under vacuum to give residue. The residue was used directly. To a solution of residue above in THE (10 mL) was added aqueous HCl (2 M, 6.0 mL). The mixture was stirred at 25° C. for 12 hours. The mixture was adjusted to pH to 8 with NaHCO₃ (saturated aqueous solution), then extracted with ethyl acetate (30 mL×3). The combined organic layers were washed with brine (30 mL), dried over Na₂SO₄, filtered and concentrated under reduced pressure to give a residue. The residue was purified by column chromatography (silica gel, petroleum ether/ethyl acetate=3/1) to give 2-methyl-5-(1,2,4-oxadiazol-3-yl) aniline (600 mg, 49%) as a white solid. ¹H NMR (400 MHz, DMSO-d₆) δ=9.60 (s, 1H), 7.33 (d, J=1.4 Hz, 1H), 7.21-7.01 (m, 2H), 5.19 (s, 2H), 2.11 (s, 3H); MS (ESI): m/z for C₉H₉N₃0 [M+H]⁺ calcd.: 176.07, [M+H]⁺ found: 176.3.

Step 2: To a solution of 2-methyl-5-(1,2,4-oxadiazol-3-yl) aniline (106 mg, 1.0 eq) in toluene (1 mL) was added Al(CH₃)₃ (2 M, 657 μL, 2.5 eq) under N₂. The mixture was stirred at 25° C. for 0.5 hour. To the mixture was added ethyl pyrazolo[1,5-a]pyridine-3-carboxylate (100 mg, 1.0 eq) and stirred at 80° C. for 2 hours. The mixture was poured into 10 mL of ice/water and adjusted to pH 9 with 1 N NaOH, then extracted with dichloromethane (30 mL×3). The combined organic layers were washed with brine (30 mL), dried over Na₂SO₄, filtered and concentrated under reduced pressure to give a residue. The residue was purified by prep-HPLC (column: C18 150×30 mm; mobile phase: [water(formic acid)-acetonitrile]; gradient:35%-65% B over 7 min) to give N-(2-methyl-5-(1,2,4-oxadiazol-3-yl)phenyl)pyrazolo[1,5-a]pyridine-3-carboxamide (18 mg) as a white solid. ¹H NMR (400 MHz, DMSO-d6) δ=9.75 (s, 1H), 9.70 (s, 1H), 8.85 (d, J=7.2 Hz, 1H), 8.78 (s, 1H), 8.24 (d, J=8.8 Hz, 1H), 8.14 (d, J=1.2 Hz, 1H), 7.82 (d, J=1.6 Hz, 1H), 7.49 (d, J=8.0 Hz, 2H), 7.13 (d, J=1.2 Hz, 1H), 2.37 (s, 3H); MS (ESI): m/z for C₁₇H₁₃N₅O₂ [M+H]⁺ calcd.: 320.11, [M+H]⁺ found: 320.3.

### Example 93—Preparation of 7-((2-hydroxy-2-methylpropoxy)methyl)-N-(2-methyl-5-(1,2,4-oxadiazol-3-yl)phenyl)imidazo[1,2-a]pyridine-3-carboxamide (I-641)

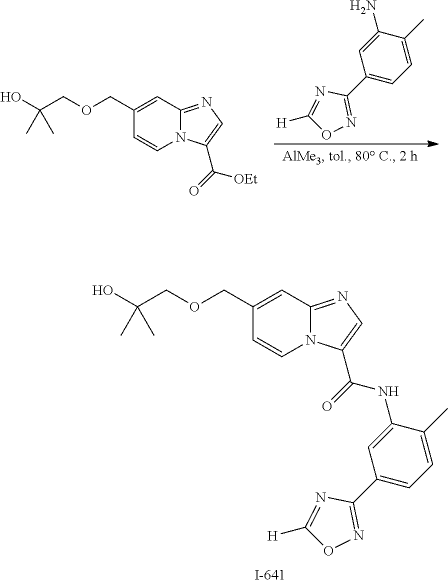 [SMILES: CCOC(=O)c1cnc2cc(COCC(C)(C)O)ccn12 ; Cc1ccc(-c2ncon2)cc1N ; Cc1ccc(-c2ncon2)cc1NC(=O)c1cnc2cc(COCC(C)(C)O)ccn12]

To a solution of 2-methyl-5-(1,2,4-oxadiazol-3-yl) aniline (90 mg, 1.0 eq) in toluene (3 mL) was added trimethylaluminium in toluene (2 M, 2.5 eq), then the reaction mixture was stirred at 25° C. for 0.5 hour. Ethyl 7-((3-hydroxy-2,2-dimethylpropoxy)methyl)imidazo[1,2-a]pyridine-3-carboxylate (150 mg, 1.0 eq) was added, then the reaction mixture was stirred at 80° C. for 1 hour. The reaction mixture was quenched with saturated aqueous ammonium chloride solution (50 mL) then extracted with dichloromethane (50 mL×3). The combined organic layers were washed with brine (25 mL×2), dried over Na₂SO₄, filtered, and concentrated under reduced pressure to give a residue. The residue was purified by prep-HPLC (neutral condition; column: Waters Xbridge 150×25 mm×5 um; mobile phase: [water(NH₄HCO3)-acetonitrile]; gradient:25%-55% B over 9 min) to give 7-((2-hydroxy-2-methylpropoxy)methyl)-N-(2-methyl-5-(1,2,4-oxadiazol-3-yl)phenyl)imidazo[1,2-a]pyridine-3-carboxamide (39 mg) as a white solid. ¹H NMR (400 MHz, CD₃OD) δ=9.46 (d, J=7.2 Hz, 1H), 9.26 (s, 1H), 8.46 (s, 1H), 8.16 (d, J=1.6 Hz, 1H), 7.94 (dd, J=1.8, 8.0 Hz, 1H), 7.73 (s, 1H), 7.49 (d, J=8.0 Hz, 1H), 7.17 (dd, J=1.6, 7.2 Hz, 1H), 4.71 (s, 2H), 3.41 (s, 2H), 2.42 (s, 3H), 1.26 (s, 6H); MS (ESI): m/z for C₂₂H₂₃N₅O₄ [M+H]⁺ calcd.: 422.1 [M+H]⁺ found: 422.1.

### Example 94—Preparation of N-[5-[5-[(1R,2S)-2-fluorocyclopropyl]-1,2,4-oxadiazol-3-yl]-2-methyl-phenyl]-7-[(2-hydroxy-2-methyl-propoxy)methyl]imidazo[1,2-a]pyridine-3-carboxamide (I-71)

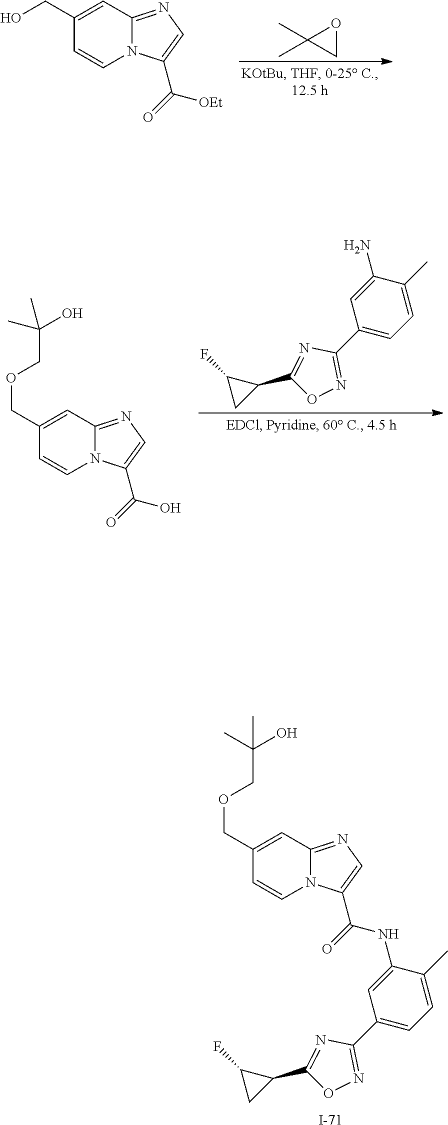

Step 1: To a stirred solution of ethyl 7-(hydroxymethyl)imidazo[1,2-a]pyridine-3-carboxylate (1.00 g, 4.54 mmol) in THF (10 mL) was added potassium tert-butoxide (1.53 g, 13.6 mmol) at 0° C. After 0.5 h, 2,2-dimethyloxirane (654 mg, 9.08 mmol, 2 eq) was added. The mixture was stirred at 25° C. for 12 h, then was concentrated in vacuo. The residue obtained was diluted with water (10 mL) and adjusted to pH 6 with aqueous hydrochloric acid (1 M). The residue was washed with ethyl acetate (30 mL×3). The aqueous phase was filtered and concentrated under reduced pressure to give a crude residue that was purified by reverse-phase HPLC (0.1% formic acid condition) to give 7-[(2-hydroxy-2-methyl-propoxy)methyl]imidazo[1,2-a]pyridine-3-carboxylic acid (200 mg, 0.756 mmol, 17%) as a yellow solid. ¹H NMR (400 MHz, DMSO-d₆) δ=9.21 (d, J=7.2 Hz, 1H), 8.20 (s, 1H), 8.13 (s, 1H), 7.71 (s, 1H), 7.32-7.05 (m, 1H), 4.64 (s, 2H), 4.57-4.44 (m, 1H), 3.26 (s, 2H), 1.12 (s, 6H).

Step 2: To a stirred solution of 7-[(2-hydroxy-2-methyl-propoxy)methyl]imidazo[1,2-a]pyridine-3-carboxylic acid (183 mg, 0.694 mmol) in pyridine (5 mL) was added 1-ethyl-3-(3-dimethylaminopropyl)carbodiimide (221 mg, 1.16 mmol). The mixture was stirred at 25° C. for 0.5 h, then 5-[5-[(1R,2S)-2-fluorocyclopropyl]-1,2,4-oxadiazol-3-yl]-2-methyl-aniline (135 mg, 0.578 mmol) was added and the mixture heated at 60° C. for 4 h. The reaction mixture was cooled then diluted with water (20 mL) and extracted with ethyl acetate (20 mL×3). The combined organic phases were washed with brine (20 mL×3), dried (Na₂SO₄), filtered and concentrated under reduced pressure to give a residue that was purified by prep-HPLC (column: Welch Ultimate XB-SiOH 250*50*10 um; mobile phase: [Hexane-ethanol (0.1% NH₃·H₂O)]; B %: 1%-35%, 15 min) and prep-HPLC (column: Waters Xbridge 150*25 mm*5 um; mobile phase: [water(NH4HCO3)-acetonitrile]; B %: 38%-68%, 8 min) to give N-[5-[5-[(1R,2S)-2-fluorocyclopropyl]-1,2,4-oxadiazol-3-yl]-2-methyl-phenyl]-7-[(2-hydroxy-2-methyl-propoxy)methyl]imidazo[1,2-a]pyridine-3-carboxamide (107 mg, 0.221 mmol, 38%) as a white solid.

¹H NMR (400 MHz, DMSO-d₆) δ=10.00 (s, 1H), 9.40 (d, J=7.6 Hz, 1H), 8.56 (s, 1H), 8.02 (d, J=1.6 Hz, 1H), 7.80-7.75 (m, 1H), 7.72 (s, 1H), 7.48 (d, J=8.0 Hz, 1H), 7.17-7.09 (m, 1H), 5.42-5.17 (m, 1H), 4.65 (s, 2H), 4.45 (s, 1H), 3.27 (s, 2H), 3.13-2.98 (m, 1H), 2.35 (s, 3H), 2.04-1.85 (m, 1H), 1.65-1.52 (m, 1H), 1.14 (s, 6H); MS (ESI): m/z for C₂₅H₂₆N₅O₄F [M+H]⁺ calculated: 480.20, [M+H]⁺ found: 480.3.

### Example 95—Analytical Data for 4-Piperidinyl-imidazo[4,5-b]pyridines and Related

### Compounds

The compounds listed in Table 2 below were prepared using experimental procedures analogous to those described in the Examples and Detailed Description. Table 2 also list each compound's ¹H NMR characterization data and mass-to-charge ratio observed by high-resolution MS or LC/MS. Chemical structures are presented in Table 1 above.

TABLE 2

| Compound | Mass Spec. | Chemical Shift Data |
|---|---|---|
| No. | m/z | (ppm, DMSO-d₆, RT, unless stated otherwise) |
| I-71 | 480.2 | δ 10.00 (s, 1H), 9.40 (d, J = 7.2 Hz, 1H), 8.56 (s, 1H), 8.02 (d, |
| | | J = 2.0 Hz, 1H), 7.77 (dd, J = 1.6, 7.6 Hz, 1H), 7.72 (s, 1H), 7.48 |
| | | (d, J = 8.0 Hz, 1H), 7.13 (dd, J = 1.6, 7.2 Hz, 1H), 5.41-5.14 |
| | | (m, 1H), 4.65 (s, 2H), 4.45 (s, 1H), 3.27 (s, 2H), 3.13-2.99 (m, |
| | | 1H), 2.35 (s, 3H), 2.00-1.88 (m, 1H), 1.63-1.53 (m, 1H), 1.14 |
| | | (s, 6H) |
| I-72 | 478.2 | δ 10.00 (s, 1H), 9.39 (d, J = 7.2 Hz, 1H), 8.56 (s, 1H), 8.02 (d, |
| | | J = 1.6 Hz, 1H), 7.77 (dd, J = 1.6, 8.0 Hz, 1H), 7.67 (s, 1H), 7.48 |
| | | (d, J = 8.0 Hz, 1H), 7.11 (dd, J = 1.6, 7.2 Hz, 1H), 5.39-5.18 |
| | | (m, 1H), 5.02 (d, J = 5.2 Hz, 1H), 4.48 (s, 2H), 4.37-4.17 (m, |
| | | 2H), 3.11-3.01 (m, 1H), 2.35 (s, 3H), 2.28-2.21 (m, 2H), 2.09- |
| | | 2.02 (m, 2H), 1.99-1.93 (m, 1H), 1.58 (dd, J = 6.4, 13.2 Hz, |
| | | 1H) |
| I-73 | 464.1 | δ 10.01 (s, 1H), 9.41 (d, J = 6.8 Hz, 1H), 8.57 (s, 1H), 8.07- |
| | | 7.99 (m, 1H), 7.81-7.74 (m, 1H), 7.71 (s, 1H), 7.48 (d, J = 8.0 |
| | | Hz, 1H), 7.15 (dd, J = 1.6, 7.2 Hz, 1H), 5.42-5.16 (m, 1H), |
| | | 4.72-4.65 (m, 3H), 4.57 (s, 2H), 4.53-4.45 (m, 2H), 3.13- |
| | | 2.95 (m, 1H), 2.35 (s, 3H), 1.99-1.90 (m, 1H), 1.64-1.51 (m, |
| | | 1H) |
| I-75 | 414.1 | δ 9.74 (s, 1H), 8.85 (d, J = 7.2 Hz, 1H), 8.78 (s, 1H), 8.24 (d, J = |
| | | 8.8 Hz, 1H), 8.11 (d, J = 1.6 Hz, 1H), 7.83-7.75 (m, 1H), 7.56- |
| | | 7.43 (m, 2H), 7.18-7.05 (m, 1H), 6.23-5.88 (m, 2H), 4.38- |
| | | 4.08 (m, 1H), 3.27 (d, J = 4.0 Hz, 1H), 3.17 (d, J = 9.2 Hz, 1H), |
| | | 2.36 (s, 3H) |
| I-76 | 444.1 | δ 9.89 (s, 1H), 9.27 (d, J = 7.2 Hz, 1H), 8.46 (s, 1H), 8.08 (d, J = |
| | | 1.6 Hz, 1H), 7.85-7.78 (m, 1H), 7.49 (d, J = 8.0 Hz, 1H), 7.16 |
| | | (d, J = 2.4 Hz, 1H), 6.93-6.80 (m, 1H), 6.26-5.82 (m, 2H), |
| | | 4.33-4.16 (m, 1H), 3.90 (s, 3H), 3.27 (d, J = 3.6 Hz, 1H), 3.18- |
| | | 3.10 (m, 1H), 2.36 (s, 3H) |
| I-77 | 392.2. | δ 10.04 (s, 1H), 9.46 (d, J = 6.8 Hz, 1H), 8.60 (s, 1H), 8.11 (d, |
| | | J = 1.6 Hz, 1H), 7.85 (dd, J = 1.6, 8.0 Hz, 1H), 7.79 (d, J = 9.2 |
| | | Hz, 1H), 7.56-7.49 (m, 2H), 7.21-7.15 (m, 1H), 5.53-5.36 |
| | | (m, 1H), 2.38 (s, 3H), 1.67-1.54 (m, 1H), 0.86-0.56 (m, 4H) |
| I-78 | 390.2 | δ 10.03 (s, 1H), 9.46 (d, J = 7.2 Hz, 1H), 8.59 (s, 1H), 8.10 (d, |
| | | J = 1.6 Hz, 1H), 7.87-7.75 (m, 2H), 7.56-7.46 (m, 2H), 7.22- |
| | | 7.13 (m, 1H), 6.21 (d, J = 6.0 Hz, 1H), 4.44-4.34 (m, 1H), 2.37 |
| | | (s, 3H), 1.42-1.26 (m, 1H), 0.66-0.36 (m, 4H) |
| I-79 | 404.1 | δ 10.04 (s, 1H), 9.46 (d, J = 7.2 Hz, 1H), 8.60 (s, 1H), 8.08 (d, |
| | | J = 1.6 Hz, 1H), 7.84-7.77 (m, 2H), 7.56-7.48 (m, 2H), 7.22- |
| | | 7.16 (m, 1H), 6.19 (d, J = 5.6 Hz, 1H), 4.91-4.76 (m, 1H), 2.84- |
| | | 2.76 (m, 1H), 2.37 (s, 3H), 2.07-1.99 (m, 2H), 1.94-1.89 (m, |
| | | 2H), 1.88-1.76 (m, 2H) |
| I-80 | 377.4 | δ 9.74 (s, 1H), 8.85 (d, J = 7.2 Hz, 1H), 8.79 (s, 1H), 8.25 (d, J = |
| | | 8.8 Hz, 1H), 8.11 (s, 1H), 7.82-7.77 (m, 1H), 7.57-7.51 (m, |
| | | 1H), 7.48 (d, J = 8.0 Hz, 1H), 7.13 (t, J = 6.8 Hz, 1H), 5.10- |
| | | 5.01 (m, 1H), 4.23-4.12 (m, 1H), 3.16-2.98 (m, 2H), 2.37 (s, |
| | | 3H), 1.22 (d, J = 6.4 Hz, 3H) |
| I-81 | 378.1 | δ 9.74 (s, 1H) 8.76-8.89 (m, 2H) 8.24 (d, J = 8.8 Hz, 1H) 8.10 |
| | | (s, 1H) 7.78 (m, 1H) 7.43-7.57 (m, 2H) 7.08-7.17 (m, 1H) |
| | | 4.95-5.14 (m, 1H) 4.08-4.24 (m, 1H) 2.98-3.14 (m, 2H) |
| | | 2.36 (s, 3H) 1.21 (d, J = 6.0 Hz, 3H) |
| I-82 | 428.1 | δ 10.16-10.25 (m, 1H) 9.58 (d, J = 7.6 Hz, 1H) 8.71 (s, 1H) |
| | | 8.04-8.11 (m, 2H) 7.83 (m, 1H) 7.50 (d, J = 8.0 Hz, 1H) 7.34- |
| | | 7.40 (m, 1H) 7.04-7.34 (m, 1H) 4.13-4.21 (m, 1H) 2.98- |
| | | 3.13 (m, 2H) 2.37 (s, 3H) 1.21 (d, J = 6.0 Hz, 3H) |
| I-83 | 428.2 | δ 10.26 (s, 1H) 9.59 (d, J = 7.2 Hz, 1H) 8.77 (s, 1H) 8.08 (br s, |
| | | 2H) 7.83 (br d, J = 7.6 Hz, 1H) 7.50 (d, J = 8.0 Hz, 1H) 7.40 (br d, |
| | | J = 7.2 Hz, 1H) 7.06-7.35 (m, 1H) 4.17 (br d, J = 6.0 Hz, 1H) |
| | | 3.00-3.10 (m, 2H) 2.37 (s, 3H) 1.21 (d, J = 6.0 Hz, 3H) |
| I-84 | 466.2 | (400 MHz, CDCl₃) δ 8.37 (d, J = 5.6 Hz, 1H), 7.33 (d, J = 2.4 |
| | | Hz, 1H), 6.63 (dd, J = 2.4, 5.6 Hz, 1H), 5.21-5.00 (m, 1H), |
| | | 4.39 (s, 2H), 2.79-2.68 (m, 1H), 2.06-2.00 (m, 2H) |
| I-85 | 478.1 | δ 10.00 (s, 1H), 9.39 (d, J = 7.6 Hz, 1H), 8.56 (s, 1H), 8.02 (d, |
| | | J = 1.6 Hz, 1H), 7.77 (dd, J = 2.0, 8.0 Hz, 1H), 7.67 (s, 1H), 7.48 |
| | | (d, J = 8.0 Hz, 1H), 7.11 (dd, J = 1.6, 7.2 Hz, 1H), 5.41-5.15 |
| | | (m, 1H), 5.04 (d, J = 6.6 Hz, 1H), 4.48 (s, 2H), 3.77-3.55 (m, |
| | | 2H), 3.11-2.98 (m, 1H), 2.60-2.54 (m, 2H), 2.35 (s, 3H), 2.01- |
| | | 1.87 (m, 1H), 1.82-1.73 (m, 2H), 1.65-1.51 (m, 1H) |
| I-86 | 436.2 | δ 9.97 (s, 1H), 9.13 (d, J = 2.4 Hz, 1H), 8.52 (s, 1H), 8.00 (d, J = |
| | | 1.6 Hz, 1H), 7.80-7.74 (m, 1H), 7.70 (d, J = 9.6 Hz, 1H), 7.48 |
| | | (d, J = 8.0 Hz, 1H), 7.37-7.30 (m, 1H), 5.44-5.13 (m, 1H), |
| | | 4.68-4.43 (m, 1H), 3.13-2.97 (m, 1H), 2.35 (s, 3H), 2.04- |
| | | 1.84 (m, 1H), 1.66-1.52 (m, 1H), 1.32 (s, 3H), 1.30 (s, 3H) |
| I-87 | 462.1 | δ 10.12 (s, 1H), 9.52 (d, J = 7.6 Hz, 1H), 8.63 (s, 1H), 8.01 (s, |
| | | 1H), 7.86 (s, 1H), 7.79 (dd, J = 1.2, 8.0 Hz, 1H), 7.49 (d, J = 8.0 |
| | | Hz, 1H), 7.27 (dd, J = 1.6, 7.2 Hz, 1H), 5.41-5.15 (m, 1H), |
| | | 3.12-2.98 (m, 1H), 2.35 (s, 3H), 2.02-1.86 (m, 1H), 1.58 (qd, |
| | | J = 6.8, 13.2 Hz, 1H) |
| I-88 | 436.3 | δ 9.88 (s, 1H), 9.24 (d, J = 7.6 Hz, 1H), 8.43 (s, 1H), 8.00 (s, |
| | | 1H), 7.79-7.73 (m, 1H), 7.47 (d, J = 8.0 Hz, 1H), 7.15 (d, J = |
| | | 2.4 Hz, 1H), 6.84-6.80 (m, 1H), 5.39-5.18 (m, 1H), 4.85- |
| | | 4.77 (m, 1H), 3.09-3.01 (m, 1H), 2.34 (s, 3H), 1.99-1.90 (m, |
| | | 1H), 1.61-1.54 (m, 1H), 1.34 (s, 3H), 1.33 (s, 3H) |
| I-89 | 408.3 | δ 9.99 (s, 1H), 9.13 (d, J = 2.4 Hz, 1H), 8.54 (s, 1H), 8.08 (s, |
| | | 1H), 7.85-7.79 (m, 1H), 7.72 (d, J = 9.6 Hz, 1H), 7.50 (d, J = |
| | | 8.0 Hz, 1H), 7.35-7.30 (m, 1H), 5.04 (d, J = 5.2 Hz, 1H), 4.23- |
| | | 4.12 (m, 1H), 3.84 (s, 3H), 3.13-2.99 (m, 2H), 2.37 (s, 3H), |
| | | 1.22 (d, J = 6.4 Hz, 3H) |
| I-90 | 440.2 | δ 9.92 (s, 1H), 9.26 (d, J = 7.6 Hz, 1H), 8.45 (s, 1H), 8.05 (d, J = |
| | | 1.2 Hz, 1H), 7.83-7.79 (m, 1H), 7.49 (d, J = 8.0 Hz, 1H), 7.16 |
| | | (d, J = 2.8 Hz, 1H), 6.90-6.85 (m, 1H), 3.89 (s, 3H), 3.23 (d, J = |
| | | 7.2 Hz, 2H), 2.35 (s, 3H), 2.26-2.19 (m, 1H), 1.80-1.72 (m, |
| | | 1H), 1.51-1.44 (m, 1H) |
| I-91 | 454.3 | δ 9.88 (s, 1H), 9.24 (d, J = 7.6 Hz, 1H), 8.43 (s, 1H), 8.00 (s, |
| | | 1H), 7.79-7.73 (m, 1H), 7.47 (d, J = 8.0 Hz, 1H), 7.15 (d, J = |
| | | 2.4 Hz, 1H), 6.84-6.80 (m, 1H), 5.39-5.18 (m, 1H), 4.85- |
| | | 4.77 (m, 1H), 3.10-2.99 (m, 1H), 2.34 (s, 3H), 2.00-1.88 (m, |
| | | 1H), 1.61-1.54 (m, 1H), 1.34 (s, 3H), 1.33 (s, 3H) |
| I-92 | 422.2 | δ 9.99 (s, 1H), 9.11 (d, J = 2.4 Hz, 1H), 8.53 (s, 1H), 8.02 (d, J = |
| | | 1.2 Hz, 1H), 7.80-7.76 (m, 1H), 7.73-7.69 (m, 1H), 7.49 (d, |
| | | J = 8.0 Hz, 1H), 7.35-7.29 (m, 1H), 5.45-5.10 (m, 1H), 4.06 |
| | | (d, J = 7.2 Hz, 2H), 3.12-3.01 (m, 1H), 2.36 (s, 3H), 2.01-1.89 |
| | | (m, 1H), 1.64-1.54 (m, 1H), 1.38 (t, J = 7.2 Hz, 3H) |
| I-93 | 422.2 | δ 9.88 (s, 1H), 9.25 (d, J = 7.6 Hz, 1H), 8.44 (s, 1H), 8.00 (d, J = |
| | | 1.6 Hz, 1H), 7.79-7.72 (m, 1H), 7.47 (d, J = 8.0 Hz, 1H), 7.14 |
| | | (d, J = 2.4 Hz, 1H), 6.89-6.81 (m, 1H), 5.43-5.12 (m, 1H), |
| | | 4.17 (q, J = 7.2 Hz, 2H), 3.12-2.97 (m, 1H), 2.34 (s, 3H), 2.02- |
| | | 1.85 (m, 1H), 1.63-1.53 (m, 1H), 1.39 (t, J = 7.2 Hz, 3H) |
| I-94 | 408.4 | δ 9.90 (s, 1H), 9.28 (d, J = 7.6 Hz, 1H), 8.46 (s, 1H), 8.07 (d, J = |
| | | 1.6 Hz, 1H), 7.85-7.76 (m, 1H), 7.49 (d, J = 8.0 Hz, 1H), 7.17 |
| | | (d, J = 2.4 Hz, 1H), 6.93-6.85 (m, 1H), 5.04 (d, J = 5.2 Hz, 1H), |
| | | 4.26-4.14 (m, 1H), 3.91 (s, 3H), 3.14-2.99 (m, 2H), 2.36 (s, |
| | | 3H), 1.25-1.19 (m, 3H) |
| I-95 | 408.2 | δ 9.89 (s, 1H), 9.27 (d, J = 7.6 Hz, 1H), 8.45 (s, 1H), 8.06 (s, |
| | | 1H), 7.84-7.75 (m, 1H), 7.48 (d, J = 8.0 Hz, 1H), 7.16 (d, J = |
| | | 2.4 Hz, 1H), 6.92-6.83 (m, 1H), 5.03 (d, J = 5.2 Hz, 1H), 4.22- |
| | | 4.10 (m, 1H), 3.90 (s, 3H), 3.12-2.98 (m, 2H), 2.35 (s, 3H), |
| | | 1.21 (d, J = 6.4 Hz, 3H) |
| I-96 | 440.2 | δ 9.92 (s, 1H), 9.26 (d, J = 7.6 Hz, 1H), 8.46 (s, 1H), 8.10-8.04 |
| | | (m, 1H), 7.85-7.77 (m, 1H), 7.49 (d, J = 8.0 Hz, 1H), 7.16 (d, |
| | | JJ = 2.4 Hz, 1H), 6.91-6.84 (m, 1H), 3.89 (s, 3H), 3.88-3.83 |
| | | (m, 1H), 3.24-3.15 (m, 2H), 3.12-3.01 (m, 2H), 2.35 (s, 3H) |
| I-97 | 362.1 | δ 10.03 (s, 1H), 9.47 (d, J = 6.8 Hz, 1H), 8.60 (s, 1H), 8.15- |
| | | 8.05 (m, 1H), 7.88-7.76 (m, 2H), 7.58-7.48 (m, 2H), 7.19 (t, |
| | | J = 6.8 Hz, 1H), 4.52-4.47 (m, 1H), 3.38-3.35 (m, 2H), 2.38 |
| | | (s, 3H) |
| I-98 | 412.1 | δ 10.04 (s, 1H), 9.45 (d, J = 7.2 Hz, 1H), 8.59 (s, 1H), 8.06 (s, |
| | | 1H), 7.87-7.74 (m, 2H), 7.58-7.47 (m, 2H), 7.24-7.13 (m, |
| | | 1H), 3.85-3.54 (m, 1H), 2.64-2.54 (m, 1H), 2.42-2.36 (m, |
| | | 3H), 2.36-2.22 (m, 1H) |
| I-99 | 396.1 | δ 10.04 (s, 1H), 9.46 (d, JJ = 7.2 Hz, 1H), 8.60 (s, 1H), 8.12 (s, |
| | | 1H), 7.87-7.82 (m, 1H), 7.79 (d, J = 9.2 Hz, 1H), 7.56-7.50 |
| | | (m, 2H), 7.18 (t, J = 6.8 Hz, 1H), 5.95-5.73 (m, 1H), 5.57 (d, |
| | | J = 4.8 Hz, 1H), 4.31-4.17 (m, 1H), 2.38 (s, 3H), 1.28-1.18 |
| | | (m, 3H) |
| I-100 | 499.3 | δ 10.57 (s, 1H), 9.55 (d, J = 7.2 Hz, 1H), 9.03 (s, 1H), 8.10 (d, |
| | | J = 1.6 Hz, 1H), 7.94 (s, 1H), 7.90-7.85 (m, 1H), 7.55 (d, J = |
| | | 8.4 Hz, 1H), 7.45 (d, J = 7.2 Hz, 1H), 5.34 (t, J = 8.0 Hz, 1H), |
| | | 4.76 (s, 2H), 3.75 (d, J = 12.0 Hz, 4H), 3.59-3.55 (m, 4H), |
| | | 3.12-3.07 (m, 1H), 2.99-2.93 (m, 1H), 2.40 (s, 3H) |
| I-101 | 396.2 | δ 9.81 (s, 1H), 9.19 (dd, J = 2.0, 4.4 Hz, 1H), 8.79 (s, 1H), 8.26 |
| | | (dd, J = 6.0, 9.6 Hz, 1H), 8.02 (d, J = 1.2 Hz, 1H), 7.75 (dd, J = |
| | | 1.6, 8.0 Hz, 1H), 7.64 (ddd, J = 2.4, 8.4, 10.0 Hz, 1H), 7.46 (d, |
| | | J = 8.0 Hz, 1H), 5.41-5.15 (m, 1H), 3.12-2.97 (m, 1H), 2.34 |
| | | (s, 3H), 2.01-1.86 (m, 1H), 1.58 (qd, J = 6.8, 13.2 Hz, 1H) |
| I-102 | 412.1 | δ 10.13 (s, 1H), 9.57-9.49 (m, 1H), 8.61 (s, 1H), 8.01 (d, J = |
| | | 1.6 Hz, 1H), 7.85 (d, J = 9.6 Hz, 1H), 7.79-7.68 (m, 1H), 7.60- |
| | | 7.58 (m, 1H), 7.49 (d, J = 8.0 Hz, 1H), 5.42-5.15 (m, 1H), |
| | | 3.12-2.99 (m, 1H), 2.35 (s, 3H), 2.02-1.87 (m, 1H), 1.59- |
| | | 1.48 (m, 1H) |
| I-103 | 396.2 | δ 10.05 (s, 1H), 9.47 (t, J = 6.8 Hz, 1H), 8.57 (s, 1H), 8.01 (s, |
| | | 1H), 7.78 (m, J = 7.3 Hz, 1H), 7.68 (dd, J = 2.5, 9.9 Hz, 1H), |
| | | 7.48 (d, J = 8.3 Hz, 1H), 7.23 (m = 2.5, 7.6 Hz, 1H), 5.41-5.13 |
| | | (m, 1H), 3.09-3.03 (m, 1H), 2.35 (s, 3H), 2.03-1.87 (m, 1H), |
| | | 1.64-1.51 (m, 1H) |
| I-104 | 412.0 | δ 10.12 (br s, 1H), 9.53 (br s, 1H), 8.61 (br s, 1H), 8.01 (br s, |
| | | 1H), 7.91-7.71 (m, 2H), 7.67-7.36 (m, 2H), 5.44-5.11 (m, |
| | | 1H), 3.05 (br s, 1H), 2.35 (br s, 3H), 2.04-1.84 (m, 1H), 1.75- |
| | | 1.47 (m, 1H) |
| I-105 | 440.2 | δ 9.98 (s, 1H), 9.38 (d, J = 7.2 Hz, 1H), 8.55 (s, 1H), 8.08 (s, |
| | | 1H), 7.82 (d, J = 6.8 Hz, 1H), 7.64 (s, 1H), 7.50 (d, J = 7.6 Hz, |
| | | 1H), 7.11 (d, J = 6.4 Hz, 1H), 5.53 (t, J = 5.6 Hz, 1H), 4.62 (d, |
| | | J = 5.6 Hz, 2H), 3.92-3.82 (m, 1H), 3.24-3.03 (m, 4H), 2.37 |
| | | (s, 3H) |
| I-106 | 433.2 | δ 10.05 (s, 1H), 9.41 (s, 1H), 8.58 (s, 1H), 8.32 (d, J = 2.0 Hz, |
| | | 1H), 7.85 (d, J = 9.6 Hz, 1H), 7.79 (m, 1H), 7.73 (d, J = 8.8 Hz, |
| | | 1H), 7.49 (d, J = 8.0 Hz, 1H), 5.42-5.09 (m, 1H), 4.16-4.02 |
| | | (m, 3H), 3.98-3.85 (m, 2H), 3.13-3.00 (m, 2H), 2.36 (s, 3H), |
| | | 2.01-1.87 (m, 1H), 1.64-1.51 (m, 1H) |
| I-107 | 428.2 | δ 10.03 (s, 1H), 9.45 (d, J = 6.8 Hz, 1H), 8.59 (s, 1H), 8.04 (d, J = |
| | | 1.6 Hz, 1H), 7.84-7.73 (m, 2H), 7.57-7.46 (m, 2H), 7.22- |
| | | 7.14 (m, 1H), 3.05-2.97 (m, 1H), 2.92-2.79 (m, 1H), 2.36 (s, |
| | | 3H), 1.70 (t, J = 7.6 Hz, 2H) |
| I-108 | 391.9 | δ 9.97 (s, 1H), 9.28 (s, 1H), 8.53 (s, 1H), 8.03 (d, J = 1.6 Hz, |
| | | 1H), 7.77-7.72 (m, 1H), 7.69 (d, J = 9.2 Hz, 1H), 7.48 (d, J = |
| | | 8.0 Hz, 1H), 7.39-7.5 (m, 1H), 5.44-5.13 (m, 1H), 3.10-3.01 |
| | | (m, 1H), 2.36 (d, J = 3.6 Hz, 6H), 2.01-1.88 (m, 1H), 1.59- |
| | | 1.54 (m, 1H) |
| I-109 | 378.2 | δ 9.73 (s, 1H), 8.85 (d, J = 6.8 Hz, 1H), 8.77 (s, 1H), 8.23 (d, J = |
| | | 8.8 Hz, 1H), 8.04 (s, 1H), 7.81-7.68 (m, 1H), 7.57-7.50 (m, |
| | | 1H), 7.46 (d, J = 8.0 Hz, 1H), 7.25-7.03 (m, 1H), 5.43-5.15 |
| | | (m, 1H), 3.16-2.94 (m, 1H), 2.35 (s, 3H), 2.01-1.82 (m, 1H), |
| | | 1.67-1.51 (m, 1H) |
| I-110 | 408.1 | δ 10.44 (s, 1H) 9.55 (d, J = 7.1 Hz, 1H) 8.94 (s, 1H) 8.08 (d, |
| | | J = 1.5 Hz, 1H) 7.92 (s, 1H) 7.83 (m, 1H) 7.51 (d, J = 8.0 Hz, 1H) |
| | | 7.44 (d, J = 8.5 Hz, 1H) 4.75 (s, 2H) 3.59-3.63 (m, 4 H) 2.67 (s, |
| | | 3H) 2.38 (s, 3H) |
| I-111 | 499.18 | δ 10.60 (s, 1H) 9.55 (d, J = 6.8 Hz, 1H) 9.05 (s, 1H) 8.10 (d, |
| | | J = 1.6 Hz, 1H) 7.94 (s, 1H) 7.88 (m, 1H) 7.55 (d, J = 8.0 Hz, 1H) |
| | | 7.46 (d, J = 8.4 Hz, 1H) 5.35 (t, J = 8.4 Hz, 1H) 4.76 (s, 2H) 3.74 |
| | | (d, J = 11.6 Hz, 4 H) 3.59-3.62 (m, 4 H) 3.07-3.13 (m, 1H) |
| | | 2.94-3.01 (m, 1H) 2.40 (s, 3H) |
| I-112 | 583.17 | (400 MHz, CDCl₃) δ 10.56 (m, 1H) 9.91-10.10 (m, 1H) 9.76- |
| | | 9.89 (m, 1H) 8.25-8.31 (m, 1H) 8.19 (s, 1H) 7.90 (d, J = 8.4 Hz, |
| | | 1H) 7.39 (d, J = 8.0 Hz, 2H) 4.79 (s, 2H) 3.87 (s, 2H) 3.75 (d, |
| | | J = 2.4 Hz, 2H) 3.62-3.70 (m, 1H) 3.05-3.15 (m, 4 H) 2.51 (s, |
| | | 3H) |
| I-113 | 476.1 | δ 10.04 (s, 1H), 9.46 (d, J = 7.2 Hz, 1H), 8.59 (s, 1H), 8.11 (d, J = |
| | | 1.6 Hz, 1H), 7.89-7.74 (m, 2H), 7.57-7.49 (m, 2H), 7.22- |
| | | 7.13 (m, 1H), 5.16-5.04 (m, 2H), 4.93-4.80 (m, 1H), 3.21- |
| | | 3.07 (m, 1H), 2.84-2.61 (m, 1H), 2.37 (s, 3H) |
| I-114 | 414.2 | δ 9.46 (d, J = 6.8 Hz, 1H), 8.59 (s, 1H), 8.09 (s, 1H), 7.75- |
| | | 7.85 (m, 2H), 7.60-7.44 (m, 2H), 7.18 (t, J = 6.8 Hz, 1H), 6.28- |
| | | 5.83 (m, 1H), 4.35-4.17 (m, 1H), 3.28 (d, J = 3.8 Hz, 1H), |
| | | 3.20-3.07 (m, 1H), 2.37 (s, 3H) |
| I-115 | 408.1 | δ 9.99 (s, 1H), 9.37 (d, J = 7.2 Hz, 1H), 8.55 (s, 1H), 8.02 (s, |
| | | 1H), 7.75-7.79 (m, 1H), 7.64 (s, 1H), 7.48 (d, J = 8.0 Hz, 1H), |
| | | 7.11 (d, J = 7.2 Hz, 1H), 5.56 (s, 1H), 5.41-5.12 (m, 1H), 4.62 |
| | | (s, 2H), 3.12-2.96 (m, 1H), 2.35-2.34 (m, 1H), 2.35 (s, 2H), |
| | | 2.02-1.86 (m, 1H), 1.53-1.64 (m, 1H) |
| I-116 | 433.2 | δ 10.07 (s, 1H), 9.41 (d, J = 7.2 Hz, 1H), 8.58 (s, 1H), 8.41 (s, |
| | | 1H), 8.02 (d, J = 1.6 Hz, 1H), 7.83-7.73 (m, 2H), 7.48 (d, J = |
| | | 8.0 Hz, 1H), 7.26 (d, J = 7.2 Hz, 1H), 5.44-5.10 (m, 1H), 4.19 |
| | | (s, 2H), 4.17-4.13 (m, 1H), 4.05 (s, 2H), 3.09-3.01 (m, 1H), |
| | | 2.35 (s, 3H), 2.02-1.85 (m, 1H), 1.58 (m, 1H) |
| I-117 | 446.2 | δ 10.02 (s, 1H), 9.53-9.26 (m, 1H), 8.57 (s, 1H), 8.10 (d, J = |
| | | 1.6 Hz, 1H), 7.86-7.80 (m, 1H), 7.72 (s, 1H), 7.50 (d, J = 8.0 |
| | | Hz, 1H), 7.18-7.10 (m, 1H), 4.74 (t, J = 5.4 Hz, 1H), 4.63 (s, |
| | | 2H), 4.48-4.32 (m, 2H), 4.32-4.24 (m, 1H), 4.20 (s, 2H), 3.59 |
| | | (s, 3H), 3.58-3.51 (m, 3H), 2.37 (s, 3H) |
| I-118 | 382.1 | δ 9.68 (s, 1H), 8.15 (s, 1H), 7.95 (d, J = 1.6 Hz, 1H), 7.81-7.72 |
| | | (m, 2H), 7.45 (d, J = 8.0 Hz, 1H), 5.40-5.20 (m, 1H), 4.23 (t, J = |
| | | 5.6 Hz, 2H), 3.11-3.00 (m, 1H), 2.81 (t, J = 6.4 Hz, 2H), |
| | | 2.30 (s, 3H), 1.93-1.80 (m, 4H), 1.57-1.59 (m, 1H) |
| I-119 | 408.1 | δ 10.41 (s, 1H) 9.22 (d, J = 2.4 Hz, 1H) 8.89 (s, 1H) 8.06 (s, 1H) |
| | | 7.93 (d, J = 9.6 Hz, 1H) 7.81-7.87 (m, 1H) 7.66 (m, 1H) 7.51 |
| | | (d, J = 8.0 Hz, 1H) 4.14-4.20 (m, 1H) 3.89 (s, 3H) 3.00-3.11 |
| | | (m, 2H) 2.38 (s, 3H) 1.21 (d, J = 6.0 Hz, 3H) |
| I-120 | 463.2 | δ 9.99 (s, 1H), 9.38 (d, J = 7.2 Hz, 1H), 8.55 (s, 1H), 8.10 (d, J = |
| | | 1.6 Hz, 1H), 7.83 (m, 1H), 7.64 (s, 1H), 7.50 (d, J = 8.0 Hz, |
| | | 1H), 7.11 (m, 1H), 5.54 (t, J = 5.6 Hz, 1H), 4.62 (d, J = 4.8 Hz, |
| | | 2H), 4.37 (d, J = 7.2 Hz, 2H), 4.32-4.25 (m, 1H), 4.25-4.15 |
| | | (m, 2H), 3.59 (s, 3H), 2.37 (s, 3H) |
| I-121 | 428.1 | δ 10.22 (s, 1H), 9.09 (d, J = 2.0 Hz, 1H), 8.57 (s, 1H), 8.22 (s, |
| | | 1H), 7.87 (m, 1H), 7.77 (d, J = 8.4 Hz, 1H), 7.73 (d, J = 9.6 Hz, |
| | | 1H), 7.34 (m, 1H), 5.43-5.18 (m, 1H), 3.83 (s, 3H), 3.14-3.03 |
| | | (m, 1H), 2.01-1.90 (m, 1H), 1.65-1.56 (m, 1H) |
| I-123 | 386.1 | δ 10.04 (s, 1H), 9.45 (d, J = 6.8 Hz, 1H), 8.59 (s, 1H), 8.02 (d, J = |
| | | 1.2 Hz, 1H), 7.78 (d, J = 8.4 Hz, 2H), 7.55-7.46 (m, 2H), |
| | | 7.17 (t, J = 6.8 Hz, 1H), 2.79 (dd, J = 4.4, 7.6 Hz, 1H), 2.35 (s, |
| | | 3H), 1.81 (dd, J = 4.2, 7.8 Hz, 1H), 1.67 (t, J = 4.2 Hz, 1H), |
| | | 1.10-0.99 (m, 3H), 0.92-0.85 (m, 1H) |
| I-124 | 424.2 | δ 10.03 (s, 1H), 9.45 (d, J = 6.8 Hz, 1H), 8.59 (s, 1H), 8.05 (s, |
| | | 1H), 7.85-7.75 (m, 2H), 7.57-7.46 (m, 2H), 7.18 (t, J = 6.8 |
| | | Hz, 1H), 3.26 (d, J = 7.2 Hz, 2H), 2.87-2.74 (m, 2H), 2.72- |
| | | 2.60 (m, 1H), 2.48-2.40 (m, 2H), 2.36 (s, 3H) |
| I-125 | 410.2 | δ 10.04 (s, 1H), 9.46 (d, J = 7.2 Hz, 1H), 8.59 (s, 1H), 8.07 (d, J = |
| | | 1.6 Hz, 1H), 7.87-7.73 (m, 2H), 7.58-7.45 (m, 2H), 7.18 (t, |
| | | J = 6.8 Hz, 1H), 3.24 (d, J = 7.2 Hz, 2H), 2.37 (s, 3H), 2.28- |
| | | 2.15 (m, 1H), 1.82-1.70 (m, 1H), 1.53-1.42 (m, 1H) |
| I-126 | 410.0 | δ 10.07 (s, 1H), 9.46 (d, J = 7.2 Hz, 1H), 8.60 (s, 1H), 8.06 (s, |
| | | 1H), 7.83-7.80 (m, 2H), 7.58-7.47 (m, 2H), 7.18 (t, J = 6.8 |
| | | Hz, 1H), 2.60-2.53 (m, 1H), 2.37 (s, 3H), 2.28-2.17 (m, 1H), |
| | | 1.71 (s, 3H) |
| I-127 | 388.2 | δ 10.03 (s, 1H), 9.45 (d, J = 6.8 Hz, 1H), 8.58 (s, 1H), 8.02 (d, J = |
| | | 1.6 Hz, 1H), 7.82-7.76 (m, 2H), 7.55-7.51 (m, 1H), 7.51- |
| | | 7.46 (m, 1H), 7.20-7.15 (m, 1H), 2.35 (s, 3H), 2.28 (t, J = 7.0 |
| | | Hz, 1H), 1.30 (d, J = 7.0 Hz, 2H), 1.27 (s, 3H), 1.16 (s, 3H) |
| I-128 | 545.2 | δ 10.05 (s, 1H), 9.43 (d, J = 7.2 Hz, 1H), 8.59 (s, 1H), 8.10 (d, J = |
| | | 1.6 Hz, 1H), 7.83 (m, 1H), 7.72 (s, 1H), 7.50 (d, J = 8.0 Hz, |
| | | 1H), 7.13 (m, 1H), 4.80 (s, 2H), 4.37 (d, J = 7.2 Hz, 2H), 4.28 |
| | | (m, 1H), 4.20 (m, 4H), 3.59 (s, 3H), 2.37 (s, 3H) |
| I-129 | 410.0 | δ 10.02 (s, 1H), 9.45 (d, J = 7.2 Hz, 1H), 8.57 (s, 1H), 7.99- |
| | | 7.89 (m, 1H), 7.78 (d, J = 9.2 Hz, 1H), 7.54-7.47 (m, 1H), |
| | | 7.36 (s, 1H), 7.16 (t, J = 6.4 Hz, 1H), 3.69 (d, J = 8.0 Hz, 1H), |
| | | 2.55 (s, 3H), 2.46-2.34 (m, 2H), 2.32-2.30 (m, 3H) |
| I-130 | 434.2 | δ 10.02 (s, 1H), 9.41 (s, 1H), 8.57 (s, 1H), 8.06-7.99 (m, 1H), |
| | | 7.87-7.80 (m, 1H), 7.79-7.71 (m, 2H), 7.48 (d, J = 8.0 Hz, |
| | | 1H), 5.42-5.16 (m, 1H), 5.02-4.94 (m, 2H), 4.64 (t, J = 6.3 |
| | | Hz, 2H), 4.43-4.33 (m, 1H), 3.13-2.99 (m, 1H), 2.35 (s, 3H), |
| | | 2.01-1.88 (m, 1H), 1.64-1.53 (m, 1H) |
| I-131 | 490.1 | δ 10.03 (s, 1H), 9.42 (d, J = 7.2 Hz, 1H), 8.58 (s, 1H), 8.02 (d, J = |
| | | 1.2 Hz, 1H), 7.80-7.76 (m, 1H), 7.72 (s, 1H), 7.48 (d, J = 8.0 |
| | | Hz, 1H), 7.16-7.12 (m, 1H), 5.40-5.16 (m, 1H), 4.80 (s, 2H), |
| | | 4.20 (q, J = 9.2 Hz, 2H), 3.14-2.96 (m, 1H), 2.35 (s, 3H), 2.03- |
| | | 1.86 (m, 1H), 1.63-1.52 (m, 1H) |
| I-132 | 378.2 | δ 9.99 (s, 1H), 9.41 (d, J = 7.2 Hz, 1H), 8.57 (s, 1H), 8.08 (d, J = |
| | | 1.6 Hz, 1H), 7.83-7.76 (m, 1H), 7.67 (s, 1H), 7.48 (d, J = 8.0 |
| | | Hz, 1H), 7.15-7.09 (m, 1H), 4.55 (s, 2H), 3.37 (s, 3H), 2.66 (s, |
| | | 3H), 2.36 (s, 3H) |
| I-133 | 446.2 | δ 10.03 (s, 1H), 9.44 (d, J = 7.2 Hz, 1H), 8.59 (s, 1H), 8.09 (d, J = |
| | | 1.6 Hz, 1H), 7.83-7.78 (m, 1H), 7.73 (s, 1H), 7.50 (d, J = 8.0 |
| | | Hz, 1H), 7.17-7.12 (m, 1H), 4.81 (s, 2H), 4.21 (q, J = 9.2 Hz, |
| | | 2H), 2.67 (s, 3H), 2.37 (s, 3H) |
| I-134 | 384.0 | δ 10.13 (s, 1H), 9.57 (d, J = 7.2 Hz, 1H), 8.69 (s, 1H), 8.10 (d, J = |
| | | 1.6 Hz, 1H), 8.04 (s, 1H), 7.85-7.79 (m, 1H), 7.50 (d, J = 8.0 |
| | | Hz, 1H), 7.36-7.04 (m, 2H), 2.67 (s, 3H), 2.38 (s, 3H) |
| I-135 | 483.0 | δ 10.14 (s, 1H), 9.56 (d, J = 7.2 Hz, 1H), 8.68 (s, 1H), 8.11 (s, |
| | | 1H), 8.04 (s, 1H), 7.88-7.81 (m, 1H), 7.51 (d, J = 8.0 Hz, 1H), |
| | | 7.36-7.01 (m, 2H), 4.42-4.33 (m, 2H), 4.32-4.25 (m, 1H), |
| | | 4.24-4.16 (m, 2H), 3.60 (s, 3H), 2.38 (s, 3H) |
| I-136 | 422.0 | δ 10.01 (s, 1H), 9.40 (d, J = 7.2 Hz, 1H), 8.56 (s, 1H), 8.02 (d, J = |
| | | 1.6 Hz, 1H), 7.77 (m, 1H), 7.67 (s, 1H), 7.48 (d, J = 8.0 Hz, |
| | | 1H), 7.12 (m, 1H), 5.39-5.17 (m, 1H), 4.54 (s, 2H), 3.36 (s, |
| | | 3H), 3.12-2.99 (m, 1H), 2.35 (s, 3H), 1.94 (s, 1H), 1.58 (m, |
| | | 1H) |
| I-137 | 447.1 | δ 10.03 (s, 1H), 9.44 (d, J = 6.8 Hz, 1H), 8.57 (s, 1H), 8.11- |
| | | 8.01 (m, 1H), 7.85-7.73 (m, 2H), 7.65 (d, J = 8.0 Hz, 1H), |
| | | 7.45 (d, J = 1.6 Hz, 2H), 7.21-7.05 (m, 1H), 4.38-4.22 (m, |
| | | 1H), 3.79-3.67 (m, 1H), 3.51 (s, 3H), 2.63-2.55 (m, 3H), 2.43 |
| | | (d, J = 2.0 Hz, 1H), 2.34 (s, 3H) |
| I-138 | 376.1 | δ 10.04 (s, 1H), 9.50-9.39 (m, 1H), 8.63-8.54 (m, 1H), 8.12 |
| | | (s, 1H), 7.91-7.83 (m, 1H), 7.79 (d, J = 9.2 Hz, 1H), 7.56- |
| | | 7.49 (m, 2H), 7.22-7.15 (m, 1H), 4.99-4.95 (m, 1H), 4.85 (t, J = |
| | | 6.4 Hz, 1H), 4.77-4.63 (m, 1H), 2.37 (s, 3H), 2.33 (s, 1H), |
| | | 2.17 (t, J = 8.4 Hz, 1H) |
| I-139 | 404.1 | δ 10.02 (s, 1H), 9.46 (d, J = 6.8 Hz, 1H), 8.59 (s, 1H), 8.07 (d, J = |
| | | 1.6 Hz, 1H), 7.82-7.76 (m, 2H), 7.54-7.48 (m, 2H), 7.18 |
| | | (m, 1H), 5.30 (s, 1H), 3.54-3.45 (m, 1H), 2.43 (m, 3H), 2.36 |
| | | (s, 3H), 2.35-2.31 (m, 1H), 1.34 (s, 3H) |
| I-140 | 477 | δ 10.01 (s, 1H), 9.41 (d, J = 7.2 Hz, 1H), 8.57 (s, 1H), 8.10 (d, J = |
| | | 1.2 Hz, 1H), 7.81-7.86 (m, 1H), 7.67 (s, 1H), 7.50 (d, J = 8.0 |
| | | Hz, 1H), 7.09-7.14 (m, 1H), 4.54 (s, 2H), 4.37 (d, J = 8.0 Hz, |
| | | 2H), 4.32-4.24 (m, 1H), 4.20 (d, J = 5.6 Hz, 2H), 3.59 (s, 3H), |
| | | 3.36 (s, 3H), 2.37 (s, 3H) |
| I-141 | 390.1 | δ 10.02 (s, 1H), 9.46 (d, J = 6.8 Hz, 1H), 8.59 (s, 1H), 8.08 (d, J = |
| | | 1.2 Hz, 1H), 7.87-7.74 (m, 2H), 7.60-7.45 (m, 2H), 7.18 (t, |
| | | J = 6.8 Hz, 1H), 5.40 (d, J = 7.2 Hz, 1H), 4.21-4.09 (m, 1H), |
| | | 3.39-3.33 (m, 1H), 2.73-2.65 (m, 2H), 2.36 (s, 3H), 2.28- |
| | | 2.19 (m, 2H) |
| I-142 | 378.1 | δ 10.03 (s, 1H), 9.46 (d, J = 7.2 Hz, 1H), 8.59 (s, 1H), 8.09- |
| | | 8.03 (m, 1H), 7.84-7.76 (m, 2H), 7.55-7.47 (m, 2H), 7.17 (m, |
| | | 1H), 5.36-5.12 (m, 1H), 2.71-2.67 (m, 1H), 2.36 (s, 3H), 1.97- |
| | | 1.82 (m, 1H), 1.62 (m, 1H). |
| I-143 | 400.2 | δ 10.02 (s, 1H), 9.46 (d, J = 6.8 Hz, 1H), 8.59 (s, 1H), 8.11- |
| | | 8.07 (m, 1H), 7.86-7.76 (m, 2H), 7.55-7.47 (m, 2H), 7.22- |
| | | 7.15 (m, 2H), 2.69 (s, 3H), 2.37 (s, 3H), 1.30 (s, 6H) |
| I-144 | 390.2 | δ 10.05 (s, 1H), 9.46 (d, J = 6.8 Hz, 1H), 8.60 (d, J = 1.2 Hz, |
| | | 1H), 8.09-8.01 (m, 1H), 7.85-7.74 (m, 2H), 7.58-7.47 (m, |
| | | 2H), 7.25-7.13 (m, 1H), 4.81-4.72 (m, 2H), 4.45 (t, J = 6.0 |
| | | Hz, 2H), 3.57-3.46 (m, 1H), 3.45-3.39 (m, 2H), 2.37 (s, 3H) |
| I-145 | 376.2 | δ 10.05 (s, 1H), 9.47 (d, J = 6.8 Hz, 1H), 8.61 (s, 1H), 8.13 (d, J = |
| | | 1.6 Hz, 1H), 7.87-7.84 (m, 1H), 7.80 (d, J = 9.2 Hz, 1H), |
| | | 7.59-7.48 (m, 2H), 7.20-7.16 (m, 1H), 6.02-5.09 (m, 1H), |
| | | 4.85-4.71 (m, 2H), 3.20-3.16 (m, 1H), 3.06-2.98 (m, 1H), |
| | | 2.39 (s, 3H) |
| I-146 | 441.1 | δ 10.04 (s, 1H), 9.46 (d, J = 6.8 Hz, 1H), 8.60 (s, 1H), 8.17 (d, J = |
| | | 7.6 Hz, 1H), 8.08 (s, 1H), 7.81 (m, 2H), 7.55-7.50 (m, 2H), |
| | | 7.19 (t, J = 6.8 Hz, 1H), 5.06-4.99 (m, 1H), 3.03 (s, 3H), 2.37 |
| | | (s, 3H), 1.59 (d, J = 7.2 Hz, 3H) |
| I-147 | 441.2 | δ 10.05 (s, 1H), 9.47 (d, J = 6.8 Hz, 1H), 8.60 (s, 1H), 8.26- |
| | | 8.13 (m, 1H), 8.12-8.05 (m, 1H), 7.89-7.73 (m, 2H), 7.58- |
| | | 7.46 (m, 2H), 7.19 (t, J = 6.8 Hz, 1H), 5.11-4.94 (m, 1H), 3.05 |
| | | (s, 3H), 2.38 (s, 3H), 1.60 (d, J = 7.2 Hz, 3H) |
| I-148 | 434.2 | δ 9.52 (d, J = 7.2 Hz, 1H), 8.46 (s, 1H), 8.21 (s, 1H), 7.89-7.73 |
| | | (m, 2H), 7.61 (s, 1H), 7.36 (d, J = 8.0 Hz, 1H), 7.18 (d, J = 7.2 |
| | | Hz, 1H), 4.95-5.20 (m, 3H), 4.78 (t, J = 6.4 Hz, 2H), 4.30 (t, J = |
| | | 7.2 Hz, 1H), 2.81-2.64 (m, 1H), 2.41 (s, 3H), 1.83 (s, 1H), |
| | | 1.55-1.66 (m, 1H) |
| I-149 | 480.2 | δ 10.00 (s, 1H), 9.40 (d, J = 7.2 Hz, 1H), 8.56 (s, 1H), 8.02 (d, J = |
| | | 2.0 Hz, 1H), 7.77 (dd, J = 1.6, 7.6 Hz, 1H), 7.72 (s, 1H), 7.48 |
| | | (d, J = 8.0 Hz, 1H), 7.13 (dd, J = 1.6, 7.2 Hz, 1H), 5.41-5.14 |
| | | (m, 1H), 4.65 (s, 2H), 4.45 (s, 1H), 3.27 (s, 2H), 3.13-2.99 (m, |
| | | 1H), 2.35 (s, 3H), 2.00-1.88 (m, 1H), 1.63-1.53 (m, 1H), 1.14 |
| | | (s, 6H) |
| I-150 | 400.2 | δ 10.02 (s, 1H), 9.46 (d, J = 6.8 Hz, 1H), 8.59 (s, 1H), 8.11- |
| | | 8.07 (m, 1H), 7.86-7.76 (m, 2H), 7.55-7.47 (m, 2H), 7.22- |
| | | 7.15 (m, 2H), 2.69 (s, 3H), 2.37 (s, 3H), 1.30 (s, 6H) |
| I-151 | 368.1 | δ 9.92 (s, 1H), 8.87 (d, J = 7.2 Hz, 1H), 8.81 (s, 1H), 8.34 (d, J = |
| | | 2.0 Hz, 1H), 8.24 (d, J = 8.8 Hz, 1H), 7.87 (dd, J = 2.0, 8.4 |
| | | Hz, 1H), 7.76 (d, J = 8.4 Hz, 1H), 7.62-7.51 (m, 1H), 7.19- |
| | | 7.09 (m, 1H), 3.07-2.97 (m, 2H), 1.34 (t, J = 7.6 Hz, 3H) |
| I-152 | 524.3 | δ 10.23 (s, 1H), 8.70 (s, 2H), 8.12-8.06 (m, 1H), 7.87 (dd, J = |
| | | 1.6, 8.0 Hz, 1H), 7.78 (d, J = 9.6 Hz, 1H), 7.53 (d, J = 8.0 Hz, |
| | | 1H), 7.46 (dd, J = 2.0, 9.6 Hz, 1H), 4.35-4.27 (m, 3H), 4.23- |
| | | 4.17 (m, 2H), 3.24-3.16 (m, 2H), 3.02 (q, J = 7.2 Hz, 2H), |
| | | 2.37 (s, 3H), 1.80-1.66 (m, 2H), 1.22 (t, J = 7.2 Hz, 3H), 1.00 |
| | | (t, J = 7.6 Hz, 3H) |
| I-153 | 391.3 | δ 10.60 (s, 1H), 9.08-9.01 (m, 1H), 8.76 (d, J = 1.6 Hz, 1H), |
| | | 8.06-8.02 (m, 1H), 7.89-7.82 (m, 2H), 7.61 (dd, J = 2.4, 9.6 |
| | | Hz, 1H), 7.51 (d, J = 8.0 Hz, 1H), 3.02 (t, J = 7.6 Hz, 4H), 2.37 |
| | | (s, 3H), 1.34 (t, J = 7.6 Hz, 3H), 1.22 (t, J = 7.2 Hz, 3H) |
| I-154 | 414.1 | δ 10.02 (s, 1H), 9.46 (d, J = 7.2 Hz, 1H), 8.59 (s, 1H), 8.13- |
| | | 8.08 (m, 1H), 7.87-7.75 (m, 2H), 7.58-7.46 (m, 2H), 7.18 (t, J = |
| | | 6.8 Hz, 1H), 6.23-5.90 (m, 2H), 4.34-4.15 (m, 1H), 3.31- |
| | | 3.28 (m, 1H), 3.17 (br d, J = 9.2 Hz, 1H), 2.37 (s, 3H) |
| I-155 | 364.1 | δ 9.7 (s, 1H) 8.8 (d, J = 6.8 Hz, 1H) 8.8 (s, 1H) 8.2 (d, J = 8.8 |
| | | Hz, 1H) 8.1-8.1 (m, 1H) 7.8 (dd, J = 7.9, 1.6 Hz, 1H) 7.5-7.6 |
| | | (m, 1H) 7.5 (d, J = 7.9 Hz, 1H) 7.1 (t, J = 6.8 Hz, 1H) 3.9 (t, J = |
| | | 6.2 Hz, 2H) 3.1 (t, J = 6.2 Hz, 2H) 2.4 (s, 3H) |
| I-156 | 407.2 | δ 10.14 (s, 1H), 9.55-9.39 (m, 1H), 8.69 (s, 1H), 8.12 (d, J = |
| | | 1.2 Hz, 1H), 7.89-7.81 (m, 2H), 7.70-7.59 (m, 1H), 7.54 (d, J = |
| | | 8.0 Hz, 1H), 7.31-7.23 (m, 1H), 5.71-5.52 (m, 1H), 5.39- |
| | | 5.32 (m, 1H), 3.82-3.61 (m, 2H), 2.95-2.60 (m, 2H), 2.38 (s, |
| | | 3H) |
| I-157 | 378.1. | δ 10.01 (s, 1H), 9.46 (d, J = 6.8 Hz, 1H), 8.59 (s, 1H), 8.08 (d, J = |
| | | 1.2 Hz, 1H), 7.84-7.76(m, 2H), 7.55-7.47 (m, 2H), 7.17 (t, |
| | | J = 6.8 Hz, 1H), 5.02 (d, J = 5.2 Hz, 1H), 4.21-4.12 (m, 1H), |
| | | 3.12-3.00 (m, 2H), 2.37 (s, 3H), 1.21 (d, J = 6.4 Hz, 3H) |
| I-158 | 377.9 | δ 10.02 (s, 1H), 9.46 (d, J = 6.8 Hz, 1H), 8.59 (s, 1H), 8.08 (s, |
| | | 1H), 7.85-7.76 (m, 2H), 7.57-7.46 (m, 2H), 7.19 (t, J = 6.8 |
| | | Hz, 1H), 5.23-4.83 (m, 1H), 4.18-4.16 (m, 1H), 3.12-2.98 |
| | | (m, 2H), 2.37 (s, 3H), 1.21 (d, J = 6.0 Hz, 3H) |
| I-159 | 407.3 | δ 10.16 (s, 1H), 9.49 (d, J = 7.2 Hz, 1H), 8.70 (s, 1H), 8.13 (d, J = |
| | | 1.6 Hz, 1H), 7.91-7.83 (m, 2H), 7.70-7.61 (m, 1H), 7.55 (d, |
| | | J = 8.0 Hz, 1H), 7.28 (dt, J = 0.8, 6.8 Hz, 1H), 5.71-5.49 (m, |
| | | 1H), 5.37 (dd, J = 5.6, 10.0 Hz, 1H), 3.85-3.71 (m, 1H), 3.69- |
| | | 3.51 (m, 1H), 3.10-2.86 (m, 1H), 2.84-2.70 (m, 1H), 2.39 (s, |
| | | 3H) |
| | 408.1 | δ 10.22 (s, 1H), 9.45 (d, J = 7.2 Hz, 1H), 8.63 (s, 1H), 8.29 (d, J = |
| | | 2.0 Hz, 1H), 7.91 (d, J = 8.4 Hz, 1H), 7.83-7.75 (m, 2H), |
| | | 7.59-7.50 (m, 1H), 7.20 (t, J = 6.4 Hz, 1H), 3.03 (q, J = 7.6 |
| | | Hz, 2H), 1.35 (t, J = 7.6 Hz, 3H) |
| I-161 | 395.2 | δ 10.31 (s, 1H), 9.91-9.63 (m, 1H), 9.46 (d, J = 7.2 Hz, 1H), |
| | | 8.70 (s, 1H), 8.37 (s, 1H), 7.97 (br d, J = 8.4 Hz, 1H), 7.85 (d, J = |
| | | 8.8 Hz, 2H), 7.62 (t, J = 8.0 Hz, 1H), 7.26 (t, J = 6.8 Hz, 1H), |
| | | 5.93 (t, J = 8.8 Hz, 1H), 4.24-4.09 (m, 1H), 4.05-3.93 (m, |
| | | 1H), 3.10-2.79 (m, 2H) |
| I-162 | 501.2 | δ 10.41 (s, 1H), 9.52 (d, J = 7.2 Hz, 1H), 8.78 (s, 1H), 8.38- |
| | | 8.29 (m, 1H), 7.99-7.87 (m, 2H), 7.82 (d, J = 8.4 Hz, 1H), |
| | | 7.73 (t, J = 8.0 Hz, 1H), 7.35 (t, J = 6.8 Hz, 1H), 5.73 (t, J = 8.4 |
| | | Hz, 1H), 4.15 (q, J = 8.4 Hz, 1H), 3.93-3.78 (m, 1H), 3.33- |
| | | 3.16 (m, 2H), 2.76-2.61 (m, 2H), 1.79-1.60 (m, 2H), 0.97 (t, J = |
| | | 7.2 Hz, 3H) |
| I-163 | 392.2. | δ 10.01 (s, 1H), 9.45 (d, J = 7.2 Hz, 1H), 8.57 (s, 1H), 7.91 (s, |
| | | 1H), 7.78 (d, J = 9.2 Hz, 1H), 7.55-7.49 (m, 1H), 7.35 (s, 1H), |
| | | 7.17 (t, J = 6.8 Hz, 1H), 5.43-5.14 (m, 1H), 3.12-3.00 (m, |
| | | 1H), 2.55 (s, 3H), 2.31 (s, 3H), 2.02-1.87 (m, 1H), 1.60-1.55 |
| | | (m, 1H) |
| I-164 | 429.1 | δ 10.32 (s, 1H), 9.45 (d, J = 7.0 Hz, 1H), 8.64 (s, 1H), 7.89 (t, J = |
| | | 7.2 Hz, 2H), 7.80 (d, J = 9.2 Hz, 1H), 7.57-7.52 (m, 1H), |
| | | 7.48-7.42 (m, 1H), 7.23-7.17 (m, 1H), 4.26 (s, 2H), 3.88 (t, J = |
| | | 12.4 Hz, 4H) |
| I-165 | 391.1 | δ 9.63 (s, 1H), 9.03 (d, J = 7.2 Hz, 1H), 8.28 (s, 1H), 8.07 (d, J = |
| | | 1.2 Hz, 1H), 7.77 (d, J = 8.0 Hz, 1H), 7.45 (d, J = 8.0 Hz, |
| | | 1H), 6.63-6.55 (m, 2H), 6.39 (d, J = 2.0 Hz, 1H), 3.17-3.07 |
| | | (m, 2H), 3.01 (q, J = 7.2 Hz, 2H), 2.34 (s, 3H), 1.34 (t, J = 7.6 |
| | | Hz, 3H), 1.21 (t, J = 7.2 Hz, 3H) |
| I-166 | 524.2 | δ 9.64 (s, 1H), 9.03 (d, J = 7.6 Hz, 1H), 8.28 (s, 1H), 8.10 (d, J = |
| | | 1.4 Hz, 1H), 7.80 (dd, J = 1.6, 7.9 Hz, 1H), 7.47 (d, J = 8.0 |
| | | Hz, 1H), 6.68-6.53 (m, 2H), 6.39 (d, J = 2.3 Hz, 1H), 4.31 (br |
| | | d, J = 2.6 Hz, 3H), 4.21 (br s, 2H), 3.23-3.17 (m, 2H), 3.12 (br |
| | | dd, J = 5.3, 6.9 Hz, 2H), 2.35 (s, 3H), 1.82-1.65 (m, 2H), 1.21 |
| | | (t, J = 7.1 Hz, 3H), 1.00 (t, J = 7.4 Hz, 3H) |
| I-428 | 333.7 | δ 9.71 (s, 1H), 8.85 (d, J = 7.2 Hz, 1H), 8.77 (s, 1H), 8.24 (d, J = |
| | | 8.8 Hz, 1H), 8.10 (d, J = 1.6 Hz, 1H), 7.77 (d, J = 8.0 Hz, |
| | | 1H), 7.56-7.50 (m, 1H), 7.46 (d, J = 8.0 Hz, 1H), 7.12 (t, J = |
| | | 6.8 Hz, 1H), 2.66 (s, 3H), 2.36 (s, 3H) |
| I-167 | 471.1 | δ 10.03 (s, 1H), 9.46 (d, J = 7.2 Hz, 1H), 8.59 (s, 1H), 8.09 (d, J = |
| | | 1.2 Hz, 1H), 7.84-7.77 (m, 2H), 7.55-7.48 (m, 2H), 7.18 (t, |
| | | J = 7.2 Hz, 1H), 4.61 (t, J = 8.0 Hz, 1H), 3.48-3.41 (m, 2H), |
| | | 3.14-3.06 (m, 1H), 2.91-2.85 (m, 1H), 2.73 (td, J = 7.2, 12.0 |
| | | Hz, 2H), 2.46-2.42 (m, 1H), 2.37 (s, 3H), 2.34-2.30 (m, 1H) |
| I-168 | 487.1 | δ 10.03 (s, 1H), 9.46 (d, J = 7.2 Hz, 1H), 8.59 (s, 1H), 8.08 (d, J = |
| | | 1.6 Hz, 1H), 7.84-7.76 (m, 2H), 7.56-7.47 (m, 2H), 7.21- |
| | | 7.15 (m, 1H), 4.64 (t, J = 8.0 Hz, 1H), 4.04 (t, J = 5.2 Hz, 2H), |
| | | 3.49-3.43 (m, 1H), 3.25-3.15 (m, 1H), 3.00-2.91 (m, 1H), |
| | | 2.90-2.81 (m, 1H), 2.55-2.54 (m, 1H), 2.46-2.42 (m, 1H), |
| | | 2.36 (s, 3H) |
| I-169 | 471.1 | δ 10.05 (s, 1H), 9.46 (d, J = 6.8 Hz, 1H), 8.59 (s, 1H), 8.08 (d, J = |
| | | 1.6 Hz, 1H), 7.87-7.76 (m, 2H), 7.56-7.48 (m, 2H), 7.21- |
| | | 7.14 (m, 1H), 5.87 (d, J = 9.2 Hz, 1H), 4.64-4.11 (m, 2H), |
| | | 3.00-2.85 (m, 1H), 2.72-2.65 (m, 1H), 2.37 (s, 3H) |
| I-170 | 378.35 | δ 10.04 (s, 1H), 9.47-9.45 (m, 1H), 8.59 (s, 1H), 8.06 (d, J = 1.6 |
| | | Hz, 1H), 7.83-7.77 (m, 2H), 7.54-7.48 (m, 2H), 7.19-7.16 (m, |
| | | 1H), 5.07 (t, J = 5.6 Hz, 1H), 3.76-3.68 (m, 2H), 2.36 (s, 3H), |
| | | 1.33 (d, J = 6.8 Hz, 3H) |
| I-171 | 378.35 | δ 10.04 (s, 1H), 9.46 (d, J = 7.2 Hz, 1H), 8.59 (s, 1H), 8.06 (d, J = |
| | | 1.6 Hz, 1H), 7.83-7.77 (m, 2H), 7.54-7.48 (m, 2H), 7.17 (t, J = |
| | | 8 Hz, 1H), 5.06 (t, J = 5.6 Hz, 1H), 3.74-3.71 (m, 2H), 2.36 |
| | | (s, 3H), 1.33 (d, J = 6.8 Hz, 3H) |
| I-172 | 354.0 | δ 10.20 (s, 1H), 9.45 (d, J = 7.2 Hz, 1H), 8.63 (s, 1H), 8.30 (d, J = |
| | | 2.0 Hz, 1H), 7.89 (dd, J = 2.0, 8.4 Hz, 1H), 7.82-7.76 (m, |
| | | 2H), 7.54 (m, 1H), 7.23-7.16 (m, 1H), 2.68 (s, 3H) |
| I-173 | 408.2 | δ 9.89 (s, 1H), 9.26 (d, J = 7.6 Hz, 1H), 8.45 (s, 1H), 8.00 (s, |
| | | 1H), 7.80-7.72 (m, 1H), 7.47 (d, J = 8.0 Hz, 1H), 7.16 (d, J = |
| | | 2.4 Hz, 1H), 6.91-6.83 (m, 1H), 5.41-5.16 (m, 1H), 3.90 (s, |
| | | 3H), 3.11-2.99 (m, 1H), 2.34 (s, 3H), 2.03-1.86 (m, 1H), 1.65- |
| | | 1.51 (m, 1H) |
| I-174 | 481.75 | δ 10.47 (s, 1H), 9.58 (d, J = 6.8 Hz, 1H), 8.91 (s, 1H), 8.06 (d, J = |
| | | 1.6 Hz, 1H), 7.98 (d, J = 8.8 Hz, 1H), 7.88-7.82 (m, 2H), 7.53 |
| | | (d, J = 8.4 Hz, 1H), 7.46-7.43 (m, 1H), 5.50 (d, J = 3.6 Hz, 1H), |
| | | 3.71 (d, J = 13.2 Hz, 1H), 3.20-3.14 (m, 1H), 3.14 (s, 3H), 2.38 |
| | | (s, 3H), 2.26-2.23 (m, 1H), 2.01-1.97 (m, 1H), 1.74-1.65 (m, |
| | | 2H), 1.58-1.43 (m, 2H) |
| I-175 | 383.2 | (400 MHz, CDCl₃) δ 9.49 (d, J = 7.2 Hz, 1H), 9.00 (m, 1H), |
| | | 8.18 (s, 1H), 7.84 (d, J = 2.4 Hz, 1H), 7.78-7.74 (m, 1H), |
| | | 7.72-7.68 (m, 1H), 7.41-7.39 (m, 1H), 7.21 (s, 1H), 7.17 (s, |
| | | 1H), 7.04-6.98 (m, 1H), 5.14-4.88 (m, 1H), 2.70-2.65 (m, |
| | | 1H), 1.86-1.72 (m, 1H), 1.62-1.58 (m, 1H) |
| I-176 | 393.2 | δ 9.78 (s, 1H), 8.66 (d, J = 2.0 Hz, 1H), 8.39 (s, 1H), 8.12-8.06 |
| | | (m, 1H), 7.79 (dd, J = 1.6, 8.0 Hz, 1H), 7.54 (d, J = 9.6 Hz, |
| | | 1H), 7.48 (d, J = 8.0 Hz, 1H), 7.19-7.16 (m, 1H), 5.79-5.73 |
| | | (m, 1H), 4.82-4.66 (m, 1H), 3.63 (d, J = 5.6 Hz, 2H), 3.30 (s, |
| | | 3H), 3.08-3.02 (m, 2H), 2.67 (s, 3H) |
| I-177 | 415.1 | δ 10.02 (s, 1H), 9.46 (d, J = 7.2 Hz, 1H), 8.59 (s, 1H), 8.08 (d, J = |
| | | 1.6 Hz, 1H), 7.85-7.76 (m, 2H), 7.56-7.47 (m, 2H), 7.18 |
| | | (m, 1H), 4.70 (t, J = 8.0 Hz, 1H), 3.38 (d, J = 5.2 Hz, 1H), 3.30- |
| | | 3.23 (m, 1H), 2.46-2.40 (m, 2H), 2.37 (s, 3H), 2.11 (m, 1H), |
| | | 0.41-0.32 (m, 2H), 0.30-0.17 (m, 2H) |
| I-178 | 456.8 | δ 10.03 (s, 1H), 9.46 (d, J = 6.8 Hz, 1H), 8.59 (s, 1H), 8.09 (d, J = |
| | | 1.6 Hz, 1H), 7.85-7.76 (m, 2H), 7.55-7.47 (m, 2H), 7.18 (t, |
| | | J = 6.8 Hz, 1H), 4.91 (t, J = 8.0 Hz, 1H), 3.56 (t, J = 7.2 Hz, |
| | | 1H), 3.50-3.45 (m, 1H), 3.44-3.39 (m, 2H), 2.56-2.52 (m, |
| | | 2H), 2.37 (s, 3H) |
| I-179 | 433.1 | δ 10.02 (s, 1H), 9.46 (d, J = 7.2 Hz, 1H), 8.59 (s, 1H), 8.09 (d, J = |
| | | 1.2 Hz, 1H), 7.86-7.74 (m, 2H), 7.56-7.45 (m, 2H), 7.18 (t, |
| | | J = 7.2 Hz, 1H), 4.53 (t, J = 8.0 Hz, 1H), 3.41 (t, J = 7.2 Hz, |
| | | 1H), 3.30-3.22 (m, 1H), 3.15-3.07 (m, 1H), 3.03 (s, 3H), 2.80- |
| | | 2.63 (m, 3H), 2.46-2.39 (m, 2H), 2.37 (s, 3H) |
| I-180 | 403.1 | δ 10.03 (s, 1H), 9.46 (d, J = 6.8 Hz, 1H), 8.59 (s, 1H), 8.09 (d, J = |
| | | 1.6 Hz, 1H), 7.85-7.76 (m, 2H), 7.55-7.48 (m, 2H), 7.18 (t, |
| | | J = 6.8 Hz, 1H), 4.44 (t, J = 8.0 Hz, 1H), 3.40 (d, J = 6.0 Hz, |
| | | 1H), 2.97 (t, J = 8.8 Hz, 1H), 2.69-2.60 (m, 2H), 2.46-2.39 |
| | | (m, 2H), 2.37 (s, 3H), 0.87 (t, J = 7.2 Hz, 3H) |
| I-429 | 402.85 | δ = 9.94 (s, 1H), 9.76(s, 1H), 8.96 (s, 1H), 8.30 (d, J = 8 Hz, |
| | | 1H), 8.02 (s, 1H), 7.76 (d, J = 8 Hz, 2H), 7.47 (d, J = 8 Hz, 1H), |
| | | 5.21-5.36 (m, 1H), 3.05 (m, 1H), 2.34 (s, 3H), 1.95(m, 2H), |
| | | 1.58 (m, 1H) |
| I-181 | 507.2 | δ 10.05 (s, 1H), 9.46 (d, J = 6.8 Hz, 1H), 8.60 (s, 1H), 8.19 (s, |
| | | 1H), 8.03 (d, J = 1.2 Hz, 1H), 7.80-7.75 (m, 2H), 7.67 (s, 1H), |
| | | 7.55-7.49 (m, 2H), 7.20-7.18 (m, 1H), 4.81-4.77 (m, 1H), 3.77 |
| | | (s, 3H), 2.37 (s, 3H), 1.48 (d, J = 7.2 Hz, 3H) |
| I-182 | 421.3 | δ 10.17(s, 1H), 9.98 (s, 1H), 8.67 (s, 1H), 8.28 (bs, 1H), 8.04 (s, |
| | | 1H), 7.97 (d, J = 9.2 Hz, 1H), 7.86 (d, J = 9.2 Hz, 1H), 7.79 (d, |
| | | J = 7.6 Hz, 1H), 7.67 (bs, 1H), 7.50 (d, J = 8.0 Hz, 1H), 5.379- |
| | | 5.373 and 5.216-5.211 (dm, J = 65.2 Hz, 1H), 3.11-3.02 (m, |
| | | 1H), 2.37 (s, 3H), 1.97-1.91 (m, 1H), 1.61-1.56 (m, 1H) |
| I-183 | 432.0 | δ 10.10 (s, 1H), 9.49 (d, J = 6.8 Hz, 1H), 8.65 (s, 1H), 8.10 (s, |
| | | 1H) 7.85-7.83 (m, 2H), 7.62 (t, J = 8 Hz, 1H), 7.51 (d, J = 8 |
| | | Hz, 1H), 7.27-7.24 (m, 1H), 6.85 (brs, 1H), 4.63 (brs, 1H), 3.44 |
| | | (dd, J1 = 15 Hz, J2 = 3.6 Hz, 2H), 3.30-3.24 (m, 1H), 2.37 (s, 3H) |
| I-184 | 519.2 | δ 10.05 (s, 1H), 9.47 (d, J = 6.8 Hz, 1H), 8.60 (s, 1H), 8.13 (s, 1 |
| | | H), 8.04 (s, 1 H), 7.99 (s, 1 H), 7.86 (d, J = 7.6 Hz, 1H), 7.79 |
| | | (d, J = 8.8 Hz, 1H), 7.54-7.51 (m, 2H), 7.18 (t, J = 6.8 Hz, 1H), |
| | | 5.47 (t, J = 8 Hz, 1H), 3.92-3.80 (m, 2H), 3.77 (s, 3H), 2.67-2.60 |
| | | (m, 2H), 2.38 (s, 3H) |
| I-185 | 516.75 | δ 10.06 (s, 1H), 9.47 (d, J = 6.8 Hz 1H), 9.02 (d, J = 2 Hz 1H), |
| | | 8.94-8.95 (m, 1H), 8.61 (s, 1H), 8.28-8.31 (m, 1H), 8.08 (d, J = |
| | | 1.6 Hz 1H), 7.73-7.86 (m, 3H) 7.53 (t, 2H), 7.19 (t, 1H), 5.43 (t, |
| | | 1H), 3.94-3.98 (m, 1H), 3.85-3.89 (m, 1H), 2.67 (m, 2H), 2.38 |
| | | (s, 3H) |
| I-186 | 515.2 | δ 10.06 (s, 1H), 9.47 (d, J = 6.8 Hz, 1H), 8.61 (s, 1H), 8.10 (s, |
| | | 1H), 7.89-7.78 (m, 5H), 7.74-7.72 (m, 2H), 7.53-7.51 (m, 2H), |
| | | 7.19 (t, J = 7.2 Hz, 1H), 5.25 (t, J = 8 Hz, 1H), 3.92-3.87 (m, 1H), |
| | | 3.77-3.71 (m, 1H), 2.67-2.61 (m, 2H), 2.38 (s, 3H) |
| I-187 | 523.2 | δ 9.97 (s, 1H), 9.52 (d, J = 7.2 Hz, 1H), 8.70 (s, 1H), 8.15 (s, |
| | | 1H), 7.85 (d, J = 8.4 Hz, 2H), 7.71-7.61 (m, 1H), 7.53 (d, J = |
| | | 8.0 Hz, 1H), 7.28 (t, J = 7.2 Hz, 1H), 5.73 (dd, J = 7.6, 8.8 Hz, |
| | | 1H), 4.21 (q, J = 8.0 Hz, 1H), 3.94-3.89 (m, 2H), 3.47-3.42 |
| | | (m, 1H), 3.40-3.31 (m, 2H), 3.30-3.20 (m, 1H), 2.80-2.67 |
| | | (m, 2H), 2.41 (s, 3H), 1.93-1.79 (m, 2H), 1.69-1.57 (m, 2H) |
| I-188 | 511.75 | δ 10.04 (s, 1H), 9.46 (d, J = 6.8 Hz, 1H), 8.60(s, 1H), 8.12 (s, |
| | | 1H), 7.84 (d, 8.0 Hz 1H), 7.89(d, J = 8.8 Hz,, 1H), 7.58- |
| | | 7.51(m, 2H), 7.18(t, J = 6.8 Hz, 1H). 5.88(t, J = 7.6 Hz, 1H), |
| | | 4.70-4.62 (m, 2H), 4.27 (q, J1 = 8.4 Hz J2 = 16.0 Hz 1H), 3.98- |
| | | 3.93(m, 1H), 3.70(s, 3H) 2.73-2.65(m, 2H), 2.37(s, 3H) |
| I-189 | 497.2 | δ 10.04 (s, 1H), 9.47 (d, J = 6.8 Hz, 1H), 8.60 (s, 1H), 8.18- |
| | | 8.07 (m, 1H), 7.85 (d, J = 7.6 Hz, 1H), 7.80 (d, J = 9.2 Hz, 1H), |
| | | 7.57-7.50 (m, 2H), 7.19 (t, J = 6.8 Hz, 1H), 5.74 (t, J = 8.0 Hz, |
| | | 1H), 4.21-4.11 (m, 1H), 3.93-3.83 (m, 1H), 3.73-3.67 (m, |
| | | 2H), 3.65-3.51 (m, 2H), 3.29 (s, 3H), 2.71-2.63 (m, 2H), 2.39 |
| | | (s, 3H) |
| I-190 | 408.3 | δ 9.98 (s, 1H), 9.12 (s, 1H), 8.53 (s, 1H), 8.01 (s, 1H), 7.78 (br |
| | | d, J = 8.0 Hz, 1H), 7.71 (br d, J = 9.6 Hz, 1H), 7.48 (br d, J = |
| | | 8.0 Hz, 1H), 7.36-7.26 (m, 1H), 5.40-5.17 (m, 1H), 3.83 (s, |
| | | 3H), 3.12-2.99 (m, 1H), 2.35 (s, 3H), 2.01-1.87 (m, 1H), 1.59 |
| | | (qd, J = 6.8, 13.2 Hz, 1H) |
| I-191 | 363.1 | δ 9.78 (s, 1H), 8.57 (s, 1H), 8.38 (s, 1H), 8.08 (d, J = 1.6 Hz, |
| | | 1H), 7.78 (dd, J = 1.6, 7.6 Hz, 1H), 7.53 (d, J = 9.6 Hz, 1H), |
| | | 7.47 (d, J = 8.0 Hz, 1H), 7.09 (dd, J = 2.4, 9.6 Hz, 1H), 5.85 (d, |
| | | J = 5.2 Hz, 1H), 3.32 (s, 3H), 2.66 (s, 3H), 2.37-2.34 (m, 3H) |
| I-430 | 377.1 | δ 9.77 (s, 1H), 8.63 (s, 1H), 8.39 (s, 1H), 8.12-8.06 (m, 1H), |
| | | 7.79 (dd, J = 1.6, 8.0 Hz, 1H), 7.54 (d, J = 9.6 Hz, 1H), 7.48 (d, |
| | | J = 8.0 Hz, 1H), 7.11 (m, 1H), 5.75 (t, J = 4.8 Hz, 1H), 3.02- |
| | | 2.95 (m, 2H), 2.67 (s, 3H), 2.36 (s, 3H), 1.22 (t, J = 7.2 Hz, 3H) |
| I-192 | 444.8 | δ 11.04-10.82 (m, 1H), 9.60 (, J = 5.6 Hz, 1H), 9.19-9.04 (m, |
| | | 1H), 8.28 (s, 1H), 8.08-8.02 (m, 1H), 8.01-7.90 (m, 2H), 7.86 |
| | | (d, J = 8.4 Hz, 1H), 7.58-7.48 (m, 1H), 5.49-5.41 (m, 1H), |
| | | 3.82 (J = 3.2 Hz, 2H), 3.22-2.95 (m, 3H) |
| I-193 | 429.1 | δ 10.03 (s, 1H), 9.46 (d, J = 6.8 Hz, 1H), 8.59 (s, 1H), 8.08 (d, J = |
| | | 1.2 Hz, 1H), 7.84-7.77 (m, 2H), 7.54-7.48 (m, 2H), 7.18 (t, |
| | | J = 6.4 Hz, 1H), 4.55 (t, J = 8.0 Hz, 1H), 3.28-3.18 (m, 2H), |
| | | 3.15-3.09 (m, 1H), 2.47-2.39 (m, 2H), 2.37 (s, 3H), 1.92- |
| | | 1.87 (m, 2H), 1.83-1.73 (m, 2H), 1.67-1.57 (m, 2H) |
| I-194 | 416.9 | δ 10.02 (s, 1H), 9.49-9.43 (m, 1H), 8.59 (s, 1H), 8.09 (d, J = |
| | | 1.6 Hz, 1H), 7.85-7.76 (m, 2H), 7.56-7.47 (m, 2H), 7.18 (t, J = |
| | | 6.8 Hz, 1H), 4.51 (t, J = 8.0 Hz, 1H), 3.38 (d, J = 4.0 Hz, 1H), |
| | | 3.01 (t, J = 8.8 Hz, 1H), 2.59-2.55 (m, 1H), 2.41-2.35 (m, |
| | | 5H), 0.87 (d, J = 6.4 Hz, 3H), 0.80 (d, J = 6.4 Hz, 3H) |
| I-195 | 451.2 | δ 9.95-9.91 (m, 3H), 8.52 (s, 1H), 8.03 (d, J = 1.6 Hz, 1H), |
| | | 7.78-7.73 (m, 2H), 7.50-7.47 (m, 2H), 5.39-5.20 (m, 1H), |
| | | 3.69 (m, 3H), 3.09-3.02 (m, 1H), 2.33 (s, 3H), 1.98-1.90 (m, |
| | | 1H), 1.63-1.57 (m, 1H) |
| I-196 | 523.85 | δ 9.56 (d, J = 6.8 Hz, 1H), 8.79 (s, 1H), 8.14 (s, 1H), 7.97-7.94 |
| | | (m, 1H), 7.88-7.70 (m, 2H), 7.56-7.51 (m, 1H), 7.44-7.40 (m, |
| | | 1H), 5.76-5.69 (m, 1H), 4.21-4.12 (m, 1H), 3.91-3.87 (m, 3H), |
| | | 3.49-3.43 (m, 1H), 3.34-3.29 (m, 2H), 2.74-2.67 (m, 2H), 2.39 |
| | | (s, 3H), 2.0-7.1.86 (m, 2H), 1.63-1.54 (m, 2H) |
| I-197 | 511.18 | δ 10.05 (s, 1H). 9.47 (d, J = 6.8 Hz, 1H), 8.60 (s, 1H), 8.12 (s, |
| | | 1H), 7.86-7.28 (m, 2H), 7.55-7.51 (m, 2H), 7.20-7.16 (m, 1H), |
| | | 7.58 (t, J = 8 Hz, 1H), 4.71-4.62 (m, 2H), 4.31-4.25 (m, 1H), |
| | | 3.99-3.94 (m, 1H), 3.71 (s, 3H), 2.73-2.66 (m, 2H), 2.38 (s, 3H) |
| I-198 | 439.4 | δ 10.18 (brs, 1H), 9.51 (d, J = 7.2 Hz, 1H), 8.71 (s, 1H), 8.11 |
| | | (d, J = 1.6 Hz, 1H), 7.90-7.85 (m, 2H), 7.71-7.67 (m, 1H), 7.54 |
| | | (d, J = 8.0 Hz, 1H), 7.32 (t, J = 7.2 Hz, 1H), 4.67 (d, J = 7.2 Hz, |
| | | 1H), 3.43-3.30 (m, 2H), 2.38-2.33 (m, 4H), 2.28-2.08 (m, 3H) |
| I-199 | 583.4 | δ 10.97 (s, 1H), 10.04 (s, 1H), 9.34 (s, 1H), 8.71 (d, J = 8.4 Hz, |
| | | 1H), 8.52 (s, 1H), 8.24-8.20 (m, 2H), 8.08 (d, J = 8.4 Hz, 1H), |
| | | 7.98 (s, 1H), 7.80-7.73 (m, 2H), 7.68-7.65 (m, 2H), 7.60 (t, J = |
| | | 8.0 Hz, 1H), 7.49 (d, J = 8.4 Hz, 1H), 7.28-7.25 (m, 1H), 5.39- |
| | | 5.21 (m, 1H), 3.13-3.04 (m, 1H), 2.31 (s, 3H), 2.00-1.93 |
| | | (m, 1H), 1.64-1.57 (m, 1H) |
| I-200 | 418.95 | δ 10.28(s, 1H), 9.12(d, J = 7.6 Hz, 1H), 8.58(s, 1H), 8.01(s, 1H), |
| | | 7.79-7.85(m, 2H), 7.50(d, J = 8 Hz, 1H), 6.94(d, J = 7.2 Hz, 1H), |
| | | 6.58(s, 1H), 5.20-5.36(m, 1H), 3.20-3.28(m, 2H), 3.02-3.11(m, |
| | | 1H), 2.34(s, 3H), 1.92-1.99(m, 1H), 1.55-1.60(m, 1H), 1.23(t, |
| | | J = 7.2 Hz, 3H) |
| I-201 | 407.3 | δ 9.66 (s, 1H), 9.02(d, J = 7.6 Hz, 1H), 8.28(s, 1H), 8.01(d, |
| | | J = 1.6 Hz,, 1H), 7.72-7.74(m, 1H), 7.45(d, J = 8 Hz, 1H), 6.65- |
| | | 6.69(m, 1H), 6.57-6.59(m, 1H), 6.36(d, J = 2 Hz, 1H), 5.19- |
| | | 5.38(m, 1H), 3.04-3.08(m, 1H), 2.75(d, J = 4.8 Hz, 3H), 2.32(s, |
| | | 3H), 1.91-1.97(m, 1H), 1.56-1.62(m, 1H) |
| I-202 | 507.0 | δ 10.01 (s, 1H), 9.12 (d, J = 2.4 Hz, 1H), 8.53 (s, 1H), 8.08 (s, |
| | | 1H), 7.84 (d, J = 8 Hz, 1H), 7.71(d, J = 10 Hz, 1H), 7.50 (d, J = 8 Hz |
| | | 1H), 7.35-7.32 (dd, J1 = 9.6 Hz, J2 = 2 Hz, 1H), 4.95 (d, J = 5.2 Hz, |
| | | 1H), 4.37-4.20(m, 5H) 4.00-3.96 (m, 1H), 3.84-3.82 (m, 2H), |
| | | 3.59 (s, 1H) 2.36 (s, 1H) 1.15 (d, J = 6.4 Hz, 3H) |
| I-203 | 507.0 | δ 10.01 (s, 1H), 9.12 (d, J = 2.4 Hz, 1H), 8.53 (s, 1H), 8.08 (d, |
| | | J = 1.6 Hz, 1H), 7.85-7.83 (dd, J1 = 8 Hz, J2 = 1.6 Hz, 1H), 7.71(d, |
| | | J = 10 Hz, 1H), 7.50 (d, J = 8 Hz 1H), 7.35-7.32 (dd, J1 = 9.6 Hz, J2 = |
| | | 2 Hz, 1H), 4.95 (d, J = 4.8 Hz, 1H), 4.37-4.20(m, 5H) 4.00- |
| | | 3.97 (m, 1H), 3.84-3.82 (m, 2H), 3.59 (s, 1H) 2.36 (s, 1H) 1.15 |
| | | (d, J = 6.4 Hz, 3H) |
| I-204 | 458.0 | δ 9.96 (s, 1H), 9.77 (s, 1H), 8.97 (s, 1H), 8.31 (d, J = 9.6 Hz, |
| | | 1H), 8.10 (s 1H), 7.84-7.75 (m, 1 H), 7.49 (d, J = 8.0 Hz, 1H), |
| | | 4.37-4.19 (m, 5H 3.59 (s, 3H). 2.35 (s, 3H) |
| I-205 | 377.1 | δ 9.94 (s, 1H), 8.84 (d, J = 2.0 Hz, 1H), 8.54 (s, 1H), 8.07 (d, J = |
| | | 1.2 Hz, 1H), 7.81 (dd, J = 1.6, 8.0 Hz, 1H), 7.70 (d, J = 9.6 |
| | | Hz, 1H), 7.58-7.45 (m, 2H), 2.91 (s, 3H), 2.91 (s, 3H), 2.67 (s, |
| | | 3H), 2.37 (s, 3H) |
| I-206 | 417.2 | (400 MHz, MeOD-d4) δ 9.24 (d, J = 2.0 Hz, 1H), 8.74 (s, 1H), |
| | | 8.13 (d, J = 1.6 Hz, 1H), 8.10-8.06 (m, 1H), 7.94-7.90 (m, |
| | | 1H), 7.89 (d, J = 10.0 Hz, 1H), 7.49 (d, J = 8.0 Hz, 1H), 3.30- |
| | | 3.28 (m, 4H), 2.66 (s, 3H), 2.42 (s, 3H), 1.84-1.74 (m, 4H), |
| | | 1.72-1.62 (m, 2H) |
| I-207 | 421.2 | δ 10.14 (s, 1H), 9.46 (d, J = 7.2 Hz, 1H), 8.68 (s, 1H), 8.32-8.30 |
| | | (m, 2H), 8.03 (d, J = 1.6 Hz, 1H), 7.80-7.77 (m, 1H), 7.72 (s, |
| | | 1H), 7.60-7.57 (m, 1H), 7.49 (d, J = 8.0 Hz, 1H), 5.38-5.20 (m, |
| | | 1H), 3.09-3.02 (m, 1H), 2.36 (s, 3H), 1.98-1.91 (m, 1H), 1.63- |
| | | 1.56 (m, 1H) |
| I-208 | 515.2 | δ 10.04 (s, 1H), 9.47 (d, J = 6.8 Hz, 1H), 8.60 (s, 1H), 8.10 (d, J = |
| | | 1.6 Hz, 1H), 7.91-7.85 (m, 2H), 7.85-7.76 (m, 3H), 7.75- |
| | | 7.69 (m, 2H), 7.57-7.48 (m, 2H), 7.18 (t, J = 7.2 Hz, 1H), 5.26 |
| | | (t, J = 8.4 Hz, 1H), 3.94-3.84 (m, 1H), 3.80-3.68 (m, 1H), |
| | | 2.66-2.60 (m, 1H), 2.44-2.42 (m, 1H), 2.38 (s, 3H) |
| I-209 | 467.4 | δ 10.05 (s, 1H), 9.46 (d, J = 7.2 Hz, 1H), 8.59 (s, 1H), 8.06 (d, |
| | | J = 1.2 Hz, 1H), 7.81 (m, 2H), 7.52 (m 2H), 7.18 (t, 1H), 5.31- |
| | | 5.34 (m, 1H), 3.47-3.52(m, 2H), 3.08 (s, 3H) 2.37-2.43 (m, |
| | | 4H), 2.02-2.08 (m, 3H) |
| I-210 | 497.4 | δ 9.64 (d, J = 7.2 Hz, 1H), 8.66 (s, 1H), 8.08 (d, J = 1.6 Hz, |
| | | 1H), 7.90-7.83 (m, 3H), 7.45-7.39 (m, 2H), 5.60 (t, J = 8.4 Hz, |
| | | 1H), 4.13 (q, J1 = 16.4 Hz, J2 = 8.4 Hz, 1H), 3.85-3.80 (m, |
| | | 1H), 3.67-3.66 (m, 2H), 3.41-3.39 (m, 1H), 3.35-3.33 (m, 1H), |
| | | 3.26 (s, 3H), 2.65-2.58 (m, 2H), 2.33 (s, 3H) |
| I-211 | 481.35 | δ 10.17 (s, 1H), 9.51 (d, J = 6.8 Hz, 1H), 8.69 (s, 1H), 8.09 (s, |
| | | 1H), 7.89-7.85 (m, 2H), 7.69-7.65 (m, 1H), 7.53-7.51 (m, 1H), |
| | | 7.31-7.28 (m, 1H), 4.28-4.20 (m, 1H), 4.06-3.99 (m, 1H), 2.93- |
| | | 2.61 (m, 10H), 2.37 (s, 3H) |
| I-212 | 444.4 | δ 10.85 (s, 1H), 9.45 (d, J = 6.8 Hz, 1H), 8.62 (s, 1H), 8.28 (s, |
| | | 1H), 8.10 (d, J = 8.4 Hz 1H), 8.03 (d, J = 7.6 Hz, 1 H), 7.83 (d, |
| | | J = 9.2 Hz, 1H), 7.58 (dd, J = 8.0, 7.6 Hz, 2H), 7.23 (dd, J = 6.8, |
| | | J = 6.8 Hz, 1H), 4.38-4.20 (m, 5H 3.59 (s, 3H) |
| I-213 | 409.14 | δ 10.06 (s, 1H), 9.45 (d, J = 6.8 Hz, 1H), 8.57(s, 1H), 7.78 (d, |
| | | J = 9.2 Hz, 1H), 7.53-7.50(m, 2H), 7.41-7.39 (m, 2H), 7.34(d, |
| | | J = 8 Hz, 1H), 7.17(t, J = 6.8 Hz, 1H), 4.58(t, J = 12 Hz, 4H), 2.26(1, |
| | | 1H)., δ = 10.06 (s, 1H), 9.45 (d, J = 6.8 Hz, 1H), 8.57(s, 1H), |
| | | 7.78 (d, J = 9.2 Hz, 1H), 7.53-7.50(m, 2H), 7.41-7.39 |
| | | (m, 2H), 7.34(d, J = 8 Hz, 1H), 7.17(t, J = 6.8 Hz, 1H), 4.58(t, |
| | | J = 12 Hz, 4H), 2.26(1, 1H) |
| I-214 | 438.16 | δ 10.02 (s, 1H), 9.45 (d, J = 7.2 Hz, 1H), 8.58 (s, 1H), 7.95 (d, |
| | | J = 1.2 Hz, 1H), 7.78 (d, J = 9.2 Hz, 1H), 7.72 (dd, J1 = 8.0 Hz, |
| | | J2 = 1.6 Hz, 1H), 7.54-7.50 (m, 1H), 7.44 (d, J = 8.0 Hz, 1H), |
| | | 7.19-7.16 (m, 1H), 3.75 (t, J = 5.6 Hz, 4H), 2.34 (s, 3H), 2.20- |
| | | 2.10 (m, 4H) |
| I-215 | 519.45 | δ 9.92 (s, 1H), 9.27 (d, J = 7.6 Hz, 1H), 8.45 (s, 1H), 8.08 (s, |
| | | 1H), 7.82 (d, J = 7.6 Hz, 1H), 7.49 (d, J = 8 Hz, 1H), 7.27(d, J = |
| | | 2.4 Hz, 1H), 6.86 (dd, J = 7.6 Hz, 2.4 Hz, 1H), 5.82 (d, J = 6 |
| | | Hz, 1H), 4.53-4.48 (m, 1H), 4.37-4.07 (m, 6H), 3.59 (s, 3H), |
| | | 2.35 (s, 3H), 2.26-2.24 (m, 1H), 2.12-2.08 (m, 1H), 1.51-1.43 |
| | | (m, 2H) |
| I-216 | 552.35 | δ 9.92 (s, 1H), 9.27 (d, J = 7.6 Hz, 1H), 8.46 (s, 1H), 8.08 (s, |
| | | 1H), 7.82 (dd, J = 8.0, 1.6 Hz, 1H), 7.49 (d, J = 8.4 Hz, |
| | | 1H), 7.16 (s, 1H), 6.88 (dd, J = 7.6, 2.8 Hz, 1H), 4.99 (bs, 1H), |
| | | 4.37-4.19 (m, 5H), 4.02-3.94 (m, 3H), 3.59 (s, 3H), 2.36 (s, 3H), |
| | | 1.18 (d, J = 6.0 Hz, 3H) |
| I-217 | 507.45 | δ 9.92 (s, 1H), 9.27 (dd, J = 7.2 Hz & 3.6 Hz, 1H), 8.46 (s, 1H), |
| | | 8.08 (s, 1H), 7.82 (d, J = 7.6 Hz, 1H), 7.49 (d, J = 8.0 Hz, |
| | | 1H), 7.16 (s, 1H), 6.89 (dd, J = 7.6, 2.8 Hz, 1H), 4.98 (bs, 1H), |
| | | 4.37-4.19 (m, 5H), 4.02-3.95 (m, 3H), 3.59 (s, 3H), 2.36 (s, 3H), |
| | | 1.18 (d, J = 6.4 Hz, 3H) |
| I-218 | 419.2 | (400 MHz, MeOD-d4) δ 9.23 (d, J = 2.0 Hz, 1H), 8.76 (s, 1H), |
| | | 8.13 (d, J = 1.6 Hz, 1H), 8.12-8.07 (m, 1H), 7.95-7.92 (m, |
| | | 1H), 7.92-7.89 (m, 1H), 7.49 (d, J = 8.0 Hz, 1H), 3.91-3.85 |
| | | (m, 4H), 3.29-3.25 (m, 4H), 2.66 (s, 3H), 2.43 (s, 3H) |
| I-219 | 349.1 | δ 9.76 (s, 1H), 8.87 (d, J = 1.6 Hz, 1H), 8.37 (s, 1H), 8.15-8.10 |
| | | (m, 1H), 7.78 (dd, J = 1.6, 7.6 Hz, 1H), 7.50 (dd, J = 8.8, 18.0 |
| | | Hz, 2H), 7.07 (dd, J = 2.0, 9.6 Hz, 1H), 5.35-5.16 (m, 2H), |
| | | 2.67 (s, 3H), 2.36 (s, 3H) |
| I-220 | 500.8 | δ 10.23 (s, 1H), 9.45 (d, J = 6.8 Hz, 1H), 8.63 (s, 1H), 8.32 (d, J = |
| | | 2.0 Hz, 1H), 7.94 (d, J = 8.4 Hz, 1H), 7.80 (d, J = 8.4 Hz, |
| | | 2H), 7.58-7.52 (m, 1H), 7.20 (t, J = 6.8 Hz, 1H), 4.36-4.32 |
| | | (m, 2H), 4.32-4.28(m, 1H), 4.26-4.18 (m, 2H), 3.23-3.16 |
| | | (m, 2H), 1.77-1.69 (m, 2H), 1.00 (t, J = 7.2 Hz, 3H) |
| I-221 | 445.0 | δ 10.81-10.65 (m, 1H), 9.57 (d, J = 6.8 Hz, 1H), 9.05-8.92 |
| | | (m, 1H), 8.29 (s, 1H), 8.04-7.94 (m, 2H), 7.91-7.81 (m, 2H), |
| | | 7.47 (t, J = 6.8 Hz, 1H), 5.38 (t, J = 8.0 Hz, 1H), 3.81-3.69 (m, |
| | | 2H), 3.36-3.20 (m, 1H), 3.14-3.07 (m, 1H), 2.98 (d, J = 8.4, |
| | | 15.2 Hz, 1H) |
| I-222 | 499.18 | δ 10.60 (s, 1H) 9.55 (d, J = 6.8 Hz, 1H) 9.05 (s, 1H) 8.10 (d, |
| | | J = 1.6 Hz, 1H) 7.94 (s, 1H) 7.88 (m, 1H) 7.55 (d, J = 8.0 Hz, 1H) |
| | | 7.46 (d, J = 8.4 Hz, 1H) 5.35 (t, J = 8.4 Hz, 1H) 4.76 (s, 2H) 3.74 |
| | | (d, J = 11.6 Hz, 4 H) 3.59-3.62 (m, 4 H) 3.07-3.13 (m, 1H) |
| | | 2.94-3.01 (m, 1H) 2.40 (s, 3H) |
| I-223 | 403.0 | δ 10.03 (s, 1H), 9.48-9.42 (m, 1H), 8.59 (s, 1H), 8.06 (d, J = |
| | | 1.6 Hz, 1H), 7.85-7.76 (m, 2H), 7.56-7.46 (m, 2H), 7.18 (t, J = |
| | | 6.8 Hz, 1H), 5.02-4.94 (m, 1H), 3.85-3.77 (m, 1H), 3.29 (s, |
| | | 1H), 2.89-2.80 (m, 1H), 2.68-2.62 (m, 1H), 2.36 (s, 3H) |
| I-224 | 389.3 | δ 10.12 (s, 1H), 9.50 (d, J = 6.8 Hz, 1H), 8.74 (s, 1H), 8.68 (s, |
| | | 1H), 8.11 (d, J = 1.6 Hz, 1H), 7.87-7.83 (m, 2H), 7.65 (t, J = 8.0 |
| | | Hz, 1H), 7.52 (d, J = 8.0 Hz, 1H), 7.28 (t, J = 7.2 Hz, 1H), 5.05- |
| | | 5.03 (m, 1H), 3.55-3.49 (m, 1H), 3.28-3.24 (m, 1H), 2.38 (s, |
| | | 3H) |
| I-225 | 469.24 | δ 10.43 (s, 1H) 9.43 (d, J = 6.8 Hz, 1H) 8.60 (s, 1H) 8.17 (s, 1H) |
| | | 8.09 (d, J = 8.0 Hz, 1H) 7.88 (d, J = 8.4 Hz, 1H) 7.80 (d, J = 9.2 |
| | | Hz, 1H) 7.51-7.58 (m, 1H) 7.11-7.42 (m, 2H) 4.16-4.50 (m, |
| | | 5 H) 3.59 (s, 3H) |
| I-226 | 552.35 | δ 10.10(s, 1H), 9.38(d, J = 8.0 Hz, 1H), 8.58(s, 1H), 7.99(s, 1H), |
| | | 7.78(d, J = 7.6 Hz, 1H), 7.48(d, J = 7.6 Hz, 1H), 7.31(s, 1H), |
| | | 7.08(d, J = 7.2 Hz, 1H), 5.20-5.35(m, 1H), 3.01-3.09(m, 1H), |
| | | 2.67(s, 3H), 2.35(s, 3H), 2.33(s, 3H), 1.89-1.99(m, 1H), 1.55- |
| | | 1.61(m, 1H) |
| I-227 | 506.2 | δ 9.99 (s, 1H), 9.12 (d, J = 2.4 Hz, 1H), 8.52 (s, 1H), 8.08 (d, |
| | | J = 1.6 Hz, 1H), 7.85-7.82 (m, 1H), 7.71(d, J = 9.6 Hz, 1H), 7.51- |
| | | 7.49 (d, J = 8.4 Hz 1H), 7.34-7.31 (dd, J1 = 9.6 Hz, J2 = 2.4 Hz, |
| | | 1H), 4.93 (d, J = 4.8 Hz, 1H), 4.39-4.20(m, 5H) 4.00-3.97 (m, |
| | | 1H), 3.84-3.82 (m, 2H), 3.59 (s, 1H) 2.36 (s, 1H) 1.16 (d, |
| | | J = 6.4 Hz, 3H) |
| I-228 | 521.45 | δ 9.89 (s, 1H), 9.27(d, J = 7.6 Hz, 1H), 8.45(s, 1H), 8.09(d, J = 1.6 |
| | | Hz, 1H), 7.82 (dd, J = 8 Hz, 1.6 Hz, 1H), 7.49 (d, J = 8 Hz, 1H), |
| | | 7.13 (d, J = 2.4 Hz, 1H), 6.88 (dd, J = 7.6 Hz, 2.8 Hz, 1H), 4.72 |
| | | (s, 1H), 4.37-4.19 (m, 5H), 2.86 (s, 2H), 3.59 (s, 3H), 2.33 (s, 3H), |
| | | 1.23 (s, 6H) |
| I-229 | 377.1 | δ 9.63 (s, 1H), 9.03 (d, J = 7.6 Hz, 1H), 8.29 (s, 1H), 8.10-8.04 |
| | | (m, 1H), 7.75 (d, J = 8.0 Hz, 1H), 7.45 (d, J = 8.0 Hz, 1H), 6.66- |
| | | 6.53 (m, 2H), 6.39 (d, J = 1.6 Hz, 1H), 3.15-3.07 (m, 2H), |
| | | 2.66 (s, 3H), 2.34 (s, 3H), 1.21 (t, J = 7.2 Hz, 3H) |
| I-230 | 414.9 | δ 10.86-10.69 (m, 1H), 9.69-9.57 (m, 1H), 9.31-9.03 (m, |
| | | 1H), 8.10 (s, 1H), 8.04 (d, J = 9.2 Hz, 1H), 7.94 (d, J = 7.2 Hz, |
| | | 1H), 7.88 (d, J = 8.0 Hz, 1H), 7.58-7.48 (m, 2H), 5.32 (t, J = |
| | | 7.6 Hz, 1H), 3.40-3.32 (m, 1H), 3.29-3.21 (m, 1H), 2.46- |
| | | 2.39 (m, 5H), 0.75 (d, J = 2.4 Hz, 4H) |
| I-231 | 414.9 | δ 10.02 (s, 1H), 9.46 (d, J = 6.8 Hz, 1H), 8.59 (s, 1H), 8.07 (s, |
| | | 1H), 7.84-7.76 (m, 2H), 7.55-7.47 (m, 2H), 7.17 (t, J = 6.8 |
| | | Hz, 1H), 4.69 (d, J = 7.6 Hz, 1H), 2.95-2.81 (m, 2H), 2.36 (s, |
| | | 3H), 2.23-2.15 (m, 1H), 2.13-2.06 (m, 1H), 0.63-0.53 (m, |
| | | 4H) |
| I-232 | 417.0 | δ 10.04 (s, 1H), 9.46 (d, J = 7.2 Hz, 1H), 8.59 (s, 1H), 8.17 (s, |
| | | 1H), 7.84-7.77 (m, 2H), 7.55-7.48 (m, 2H), 7.18 (t, J = 6.8 |
| | | Hz, 1H), 4.64 (d, J = 8.4 Hz, 1H), 2.36 (s, 3H), 2.33-2.15 (m, |
| | | 2H), 1.77-1.60 (m, 2H), 1.16 (d, J = 8.0 Hz, 6H) |
| I-233 | 516.35 | δ 10.04 (s, 1H), 9.47(d, J = 7.2 Hz, 1H), 9.02(d, J = 2.0 Hz, 1H), 8.94- |
| | | 8.93 (m, 1H), 8.60 (s, 1H), 8.30-8.28 (m, 1H), 8.09 (s, 1H), |
| | | 7.83-7.75 (m, 3H), 7.54-7.51 (m, 2H), 7.20-7.17 (m, 1H), 5.43 |
| | | (t, J = 8.4 Hz, 1H), 3.99-3.94 (m, 1H), 3.90-3.84 (m, 1H), 2.71- |
| | | 2.58 (m, 2H), 2.38 (s, 3H) |
| I-234 | 467.2 | δ 9.63 (d, J = 7.2 Hz, 1H), 8.64(s, 1H), 8.07(d, J = 1.2 Hz, 1H), 7.88 |
| | | (d, J = 4.0 Hz, 2H), 7.85-7.82 (m, 1H), 7.43-7.39 (m, 2H), |
| | | 5.56(t, J = 8.0 Hz, 1H), 4.16(q, J1 = 8.8 Hz, J2 = 16.0 Hz 1H), 3.78- |
| | | 3.73 (m, 1H), 3.05-3.01 (m, 2H), 2.66-2.62 (m, 2H), 2.33 (s, |
| | | 3H), 1.20 (t, J = 7.6 Hz, 3H |
| I-235 | 496.15 | δ 10.03 (s, 1H), 9.45 (d, J = 6.8 Hz, 1H), 8.59 (s, 1H), 8.10 (d, J = |
| | | 1.6 Hz, 1H), 7.84 (dd, J = 8 Hz, 2 Hz, 1H), 7.78 (d, J = 8.8 |
| | | Hz, 1H), 7.54-7.50 (m, 2H), 7.19-7.16 (m, 1H), 4.35-4.21(m, |
| | | 5H), 3.69 (t, J = 6 Hz, 2H), 3.53 (t, J = 6 Hz, 2H), 3.27 (s, 3H), |
| | | 2.33 (s, 3H) |
| I-236 | 511.2 | δ 10.16 (s, 1H), 9.51 (d, J = 7.2 Hz, 1H), 8.60 (s, 1H), 8.11 (s, |
| | | 1H), 7.88-7.85 (m, 2H), 7.67 (t, J = 8 Hz, 1H), 7.52 (d, J = 8.4 |
| | | Hz, 1H), 7.30 (t, J = 7.2 Hz, 1H), 4.59 (s, 2H), 4.47-4.42 (m, |
| | | 2H), 4.37-4.30 (m, 3H), 3.66 (s, 3H), 2.33 (s, 3H) |
| I-237 | 441.2 | δ 10.03(d, J = 7.2 Hz, 1H), 9.46(d, J = 6.8 Hz, 1H), 8.60(s, 1H), 8.18 |
| | | (d, J = 7.6 Hz, 1H), 8.09 (d, J = 1.6 Hz, 1H), 7.84-7.78 (m, 2H), |
| | | 7.54-7.50 (m, 2H), 7.20-7.16 (m, 1H), 5.06-4.99 (m, 1H), 3.04 |
| | | (s, 3H), 2.37 (s, 3H), 1.57 (d, J = 7.2 Hz, 3H) |
| I-238 | 441.2 | δ 10.03(d, J = 7.2 Hz, 1H), 9.46(d, J = 6.8 Hz, 1H), 8.60(s, 1H), 8.18 |
| | | (d, J = 7.6 Hz, 1H), 8.09 (d, J = 1.6 Hz, 1H), 7.84-7.78 (m, 2H), |
| | | 7.54-7.50 (m, 2H), 7.20-7.16 (m, 1H), 5.06-4.99 (m, 1H), 3.04 |
| | | (s, 3H), 2.37 (s, 3H), 1.57 (d, J = 7.2 Hz, 3H) |
| I-239 | 521.4 | δ 10.19 (s, 1H), 9.19 (d, J = 2.4 Hz 1H), 8.68 (s, 1H), 8.08 (d, J = |
| | | 1.6 Hz 1H), 7.87-7.83 (m, 2H), 7.56-7.51 (m, 2H), 4.38-4.19 (m, |
| | | 5H), 3.79 (s, 2H) 3.59 (s, 3H), 2.37 (s, 3H), 1.24 (s, 6H) |
| I-240 | 434.4 | δ 10.69 (s, 1H), 9.25(d, J = 6.8 Hz, 1H), 8.24 (s, 1H), 8.04 (d, J = |
| | | 9.2 Hz, 1H), 7.85(d, J = 8.0 Hz, 1H), 7.63(td, J = 6.8 Hz, J = |
| | | 0.8 Hz, 1H), 7.51(d, J = 8.0 Hz, 1H), 7.26(t, J = 7.6 Hz, 1H), |
| | | 4.38-4.2(m, 5H) |
| I-241 | 439.05 | δ 10.07 (s, 1H), 9.45 (d, J = 6.8 Hz, 1H), 8.60 (s, 1H), 8.05 (s, |
| | | 1H), 7.77-7.83 (m, 2H), 7.49-7.54 (m, 2H), 7.18 (t, J = 6.8 Hz, |
| | | 1H), 3.77-3.80 (m, 1H), 3.37-3.42 (m, 1H), 3.21-3.28 (m, 1H), |
| | | 2.98-3.02 (m, 1H), 2.36 (s, 3H), 1.66 (s, 3H) |
| I-242 | 481.0 | δ 10.03 (s, 1H), 9.46 (d, J = 6.8 Hz, 1H), 8.60 (s, 1H), 8.12 (d, J = |
| | | 1.6 Hz, 1H), 7.85-7.78 (m, 2H), 7.54-7.50 (m, 2H), 7.19-7.16 |
| | | (m, 1H), 5.71 (t, J = 8.0 Hz, 1H), 4.17-4.11 (m, 1H), 3.85-3.80 |
| | | (m, 1H), 3.34-3.21 (m, 2H), 2.71-2.65 (m, 2H), 2.38 (s, 3H), |
| | | 1.75-1.66 (m, 2H), 0.97 (t, J = 7.2 Hz, 3H) |
| I-243 | 425.0 | δ 8.59 (d, J = 6.8 Hz, 1 H), 8.54 (s, 1H), 8.18 (d, J = 9.2 Hz, 1 H), |
| | | 8.11 (d, J = 1.2 Hz, 1 H), 7.84 (dd, J = 1.6 Hz, 8.0 Hz, 1H), |
| | | 7.44-7.39 (m, 2H), 7.02 (t, J = 6.8 Hz, 1H), 5.48 (t, J = 8.4 Hz, |
| | | 1H), 3.96-3.82 (m, 2 H), 3.18-3.08 (m, 1H), 3.06-2.9 (m, 1H), |
| | | 2.33 (s, 3H) |
| I-244 | 400.2 | δ 9.54 (d, J = 7.2 Hz, 1H), 8.55 (s, 1H), 8.19 (s, 1H), 8.13 (d, |
| | | J = 1.6 Hz, 1H), 7.89 (dd, J1 = 8 Hz, J2 = 1.6 Hz, 1H), 7.43-7.41 |
| | | (m, 1H), 7.27 (dd, J1 = 7.2 Hz, J2 = 1.6 Hz, 1H), 5.83 (t, J = 8.4 Hz, |
| | | 1H),), 4.22-4.17 (m, 1H), 4.11-4.07 (m, 1H), 3.04-2.95 (m, |
| | | 2H), 2.34 (s, 3H) |
| I-245 | 421.0 | δ 9.68 (s, 1H), 9.14(d, J = 8.0 Hz, 1H), 8.34 (s, 1H), 8.02 (s, |
| | | 1H), 7.73(dd, J = 1.6 Hz, 1H), 7.45(d, J = 8.0 Hz, 1H), 6.88- |
| | | 6.91 (m, 1H), 6.59 (d, J = 2.4 Hz, 1H), 5.38-5.35 and 5.22-5.19 |
| | | (dm, J = 63.2 Hz, 1H), 3.05(s, 6H), 2.34 (s, 3H), 1.98-1.91 |
| | | (m, 1H), 1.63-1.54(m, 1H) |
| I-246 | 507.0 | δ 10.01 (s, 1H), 9.12 (d, J = 2 Hz, 1H), 8.53 (s, 1H), 8.08 (d, |
| | | J = 1.6 Hz, 1H), 7.85-7.82 (m, 1H), 7.71(d, J = 10 Hz, 1H), 7.51- |
| | | 7.49 (d, J = 8.4 Hz 1H), 7.35-7.32 (dd, J1 = 9.6 Hz, J2 = 2.4 Hz, |
| | | 1H), 4.93 (d, J = 4.8 Hz, 1H), 4.37-4.19(m, 5H) 4.00-3.97 (m, |
| | | 1H), 3.84-3.82 (m, 2H), 3.59 (s, 1H) 2.36 (s, 1H) 1.16 (d, |
| | | J = 6.4 Hz, 3H) |
| I-247 | 493.0 | δ 9.99 (s, 1H), 9.13 (d, J = 2.4 Hz, 1H), 8.53 (s, 1H), 8.08 (d, |
| | | J = 1.6 Hz, 1H), 7.85-7.82 (m, 1H), 7.71(d, J = 10 Hz, 1H), 7.51- |
| | | 7.49 (m, 1H), 7.35-7.32 (dd, J1 = 9.6 Hz, J2 = 2.4 Hz, 1H), 4.94 (t, |
| | | J = 5.6 Hz, 1H), 4.37-4.19(m, 5H) 4.03-4.00 (m, 2H), 3.76-3.72 |
| | | (m, 2H), 3.59 (s, 1H) 2.36 (s, 1H) |
| I-248 | 439.1 | δ 10.04 (s, 1H), 9.46 (d, J = 8 Hz, 1H), 8.59 (s, 1H), 8.09 (s, |
| | | 1H), 8.01 (s, 1H), 7.80 (m, 2H), 7.53 (t, J = 8 Hz 2H), 7.18 (t, J = |
| | | 7.1 Hz 1H), 5.11 (t, J = 8 Hz, 1H), 3.37-3.43 (m, 1H) 3.15-3.22 |
| | | (m, 1H), 2.90-2.94 (m, 1H), 2.71-2.77 (m, 1H), 2.33 (s, 3H) |
| I-249 | 439.1 | δ 10.04 (s, 1H), 9.46 (d, J = 8 Hz, 1H), 8.59 (s, 1H), 8.09 (s, |
| | | 1H), 8.01 (s, 1H), 7.80 (m, 2H), 7.53 (t, J = 8 Hz 2H), 7.18 (t, J = |
| | | 7.1 Hz 1H), 5.11 (t, J = 8 Hz, 1H), 3.37-3.43 (m, 1H) 3.15-3.22 |
| | | (m, 1H), 2.90-2.94 (m, 1H), 2.71-2.77 (m, 1H), 2.33 (s, 3H) |
| I-250 | 375.2 | δ 10.11 (s, 1H), 9.74 (bs, 1H), 9.48 (d, J = 6.8 Hz, 1H), 8.67 (s, |
| | | 1H), 8.15 (s, 1H), 7.89-7.84 (m, 2H), 7.64-7.60 (m, 1H), 7.55 (d, |
| | | J = 8.0 Hz, 1H), 7.25 (t, J = 6.8 Hz, 1H), 5.91 (t, J = 8.4 Hz, |
| | | 1H), 4.19-4.12 (m, 1H), 4.03-3.96 (m, 1H), 3.03-2.98 (m, 1H), |
| | | 2.90-2.86 (m, 1H), 2.37 (s, 3H) |
| I-251 | 481.0 | δ 9.75 (s, 1H), 8.85 (d, J = 7.6 Hz, 1H), 8.78 (s, 1H), 8.24 (d, |
| | | J = 8.8 Hz, 1H), 8.12(d, J = 1.6 Hz, 1H), 7.81(d, d, J1 = 9.6 Hz |
| | | J2 = 1.6 Hz, 1H), 7.55-7.48 (m, 2H), 7.14-7.10(m, 1H), 4.34- |
| | | 4.27 (m, 3H), 4.24-4.17 (m, 2H), 3.32-3.18 (m, 2H), 2.37(s, |
| | | 3H), 1.78-1.68(m, 2H), 1.00 (t, J = 7.2 Hz, 3H) |
| I-252 | 359.2 | δ 10.25 (s, 1H), 9.54-9.52 (m, 1H), 8.76 (s, 1H), 8.57 (s, 1H), 8.07 |
| | | (d, J = 1.6 Hz, 1H), 7.82 (dd, J1 = 8 Hz, J2 = 1.6 Hz, 1H), 7.51- |
| | | 7.46 (m, 2H), 2.66 (s, 3H), 2.36 (s, 3H) |
| I-253 | 426.95 | δ = 10.46 (s, 1H), 9.46 (d, J = 6.8 Hz, 1H), 8.60 (s, 1H), 8.10 |
| | | (bs, 2H), 7.83-7.78 (m, 2H), 7.54-7.50 (m, 2H), 7.18 (t, J = 6.8 |
| | | Hz, 1H), 4.63 (s, 2H) 3.05 (s, 3H), 2.37 (s, 3H) |
| I-254 | 449.4 | δ 9.66 (s, 1H), 9.48 (d, J = 6.8 Hz, 1H), 8.63 (s, 1H), 8.47 (d, |
| | | J = 2.4 Hz, 1H), 7.88 (dd, J1 = 2.4 Hz, J2 = 8.8 Hz, 1H)7.78 (d, |
| | | J = 8.8 Hz, 1H), 7.55-7.51 (m, 1H), 7.29 (d, J = 8.8 Hz, 1H), 7.20- |
| | | 7.17 (m, 1H), 4.39-4.21 (m, 4H), 3.95 (s, 3H), 3.60 (s, 3H) |
| I-255 | 471.1 | δ 9.5 (s, 1H), 9.14 (d, J = 8.4 Hz, 1H), 8.35 (s, 1H), 8.04(d, |
| | | J = 1.6 Hz, 1H), 7.72(dd, J1 = 1.6 Hz, J2 = 8.0 Hz, 1H), 7.42(d, |
| | | J = 7.6 Hz, 1H), 7.20(s, 1H), 6.83(s, 1H), 5.12-5.32(m, 1H), 2.91- |
| | | 2.95(m, 4H), 2.34(s, 3H), 1.87-1.92(m, 2H), 1.54-1.59(m, 1H) |
| I-256 | 378.4 | δ 10.07 (s, 1H), 9.45 (d, J = 6.8 Hz, 1H), 8.60 (s, 1H), 8.05 (s, |
| | | 1H), 7.77-7.83 (m, 2H), 7.49-7.54 (m, 2H), 7.18 (t, J = 6.8 Hz, |
| | | 1H), 3.77-3.80 (m, 1H), 3.37-3.42 (m, 1H), 3.21-3.28 (m, 1H), |
| | | 2.98-3.02 (m, 1H), 2.36 (s, 3H), 1.66 (s, 3H) |
| I-257 | 378.4 | δ 10.06 (s, 1H), 9.47 (d, J = 6.8 Hz, 1H), 8.62 (s, 1H), 8.08 (s, |
| | | 1H), 7.83-7.79 (m, 2H), 7.56-7.48 (m, 2H), 7.20 (t, J = 5.6 Hz, |
| | | 1H), 5.05 (bs, 1H), 4.21-4.13 (m, 1H), 3.12-2.99 (m, 2H), 2.37 |
| | | (s, 3H), 1.30-1.09 (m, 3H) |
| I-258 | 441.2 | δ 10.04 (s, 1H), 9.46 (d, J = 6.8 Hz, 1H), 8.60 (s, 1H), 8.12 (d, J = |
| | | 1.2 Hz, 1H), 7.85-7.78 (m, 2H), 7.53-7.51 (m, 2H), 7.20-7.10 |
| | | (m, 1H), 5.18 (s, 2H), 2.86 (s, 6H), 2.38 (s, 3H) |
| I-259 | 497.1 | δ 10.48 (s, 1H), 9.93(s, 1H), 9.33 (d, J = 7.6 Hz, 1H), 8.48 (s, |
| | | 1H), 8.01(d, J = 1.6 Hz, 1H), 7.75-7.77(m, 1H), 7.47(d, J = 8 Hz, |
| | | 1H), 7.41(d, J = 1.6 Hz, 1H), 7.05(dd, J1 = 2 Hz, J2 = 7.6 Hz, 1H), |
| | | 5.19-5.38(m, 1H), 3.03-3.08(m, 1H), 2.86-2.87(m, 1H), 2.33(s, |
| | | 3H), 1.90-1.98(m, 1H), 1.54-1.62(m, 1H), 1.01-1.03(m, 4H). |
| I-260 | 539.0 | δ 10.23(s, 1H), 9.40(d, J = 7.2 Hz, 1H), 8.66(s, 1H), 7.98-8.03(m, |
| | | 2H), 7.81-7.82(m, 2H), 7.52(d, J = 2.0 Hz, 1H), 7.48(d, J = 8.0 Hz, |
| | | 1H), 7.18-7.20(m, 2H), 5.18-5.37(m, 1H), 3.01-3.10(m, 1H), |
| | | 2.36(s, 3H), 1.91-1.98(m, 1H), 1.53-1.62(m, 1H) |
| I-261 | 521.5 | δ 9.90 (s, 1H), 9.29 (d, J = 7.6 Hz, 1H), 8.45 (s, 1H), 8.09 (s, |
| | | 1H), 7.82 (dd, J = 7.6 Hz, 1H), 7.49 (d, J = 8 Hz, 1H), 7.13 (s, |
| | | 1H), 6.89 (d, J = 6.4 Hz, 1H), 4.74 (s, 1H), 4.37-4.20 (m, 5H), |
| | | 3.86 (s, 2H), 3.59 (s, 3H), 2.36 (s, 3H), 1.23 (s, 6H) |
| I-262 | 507.45 | δ 9.90 (s, 1H), 9.27 (d, J = 7.6 Hz, 1H), 8.45 (s, 1H), 8.08 (s, |
| | | 1H), 7.83(d, J = 7.6 Hz, 1H), 7.50(d, J = 7.6 Hz, 1H), 7.15(s, 1H), |
| | | 6.87(d, J = 7.2 Hz, 1H), 4.98 (d, J = 4.4 Hz, 1H), 4.44-4.16 (m, |
| | | 5H), 4.01-3.92 (m, 3H), 3.64 (s, 3H), 2.35 (s, 3H), 1.18 (d, J = 6 |
| | | Hz, 3H) |
| I-263 | 507.15 | δ 10.14 (s, 1H), 9.35 (d, J = 7.6 Hz, 1H), 8.61 (s, 1H), 8.08 (d, J = |
| | | 1.6 Hz, 1H), 7.85 (dd, J = 8 Hz, 1.6 Hz, 1H), 7.51 (d, J = 8 |
| | | Hz, 1H), 7.28 (d, J = 2.4 Hz, 1H), 7.08-7.06 (m, 1H), 4.39-4.36 |
| | | (m, 2H), 4.29-4.26 (m, 2H), 4.21-4.28 (m, 2H), 4.05-4.00 (m, |
| | | 3H), 3.59 (s, 3H), 2.36 (s, 3H), 2.19 (d, J = 6 Hz, 3H |
| I-264 | 493.45 | δ 10.13 (s, 1H), 9.35 (d, J = 7.6 Hz, 1H), 8.60 (s, 1H), 8.08 (d, J = |
| | | 1.6 Hz, 1H), 7.85 (dd, J = 8 Hz, 2 Hz, 1H), 7.51 (d, J = 8 Hz, |
| | | 1H), 7.28 (d, J = 2 Hz, 1H), 7.08-7.06 (m, 1H), 4.39-4.35 (m, |
| | | 2H), 4.31-4.18 (m, 5H), 3.80-3.78 (m, 3H), 3.59 (s, 3H), 2.36 |
| | | (s, 3H) |
| I-265 | 458.05 | δ 10.00 (s, 1H), 9.08 (d, J = 7.2 Hz, 1H), 8.93 (s, 1H), 8.72 (s, |
| | | 1H), 8.11 (d, J = 1.6 Hz, 1H), 7.84(dd, J1 = 8.0 Hz, J2 = 1.6 Hz, |
| | | 1H), 7.51-7.44 (m, 2 H), 3.59(s, 3H), 2.36 (s, 3H) |
| I-266 | 450.45 | δ 10.05 (s, 1H), 9.46 (d, J = 6.8 Hz, 1H), 8.60 (s, 1H), 8.07(d, J = |
| | | 1.6, 1H), 7.83-7.78 (m, 2H), 7.55-7.48 (m, 2H), 7.20-7.16 (m, |
| | | 1H), 3.91-3.83 (m, 1H), 2.77 (t, J = 12.4 Hz, 2H), 2.68-2.55 |
| | | (m, 6H), 2.37 (s, 3H) |
| I-267 | 385.35 | δ 10.03 (s, 1H), 9.46-9.44 (m, 1H), 8.59 (s, 1H), 8.03 (d, J = 1.2 |
| | | Hz, 1H), 7.80-7.77 (m, 2H), 7.54-7.48 (m, 2H), 7.19-7.16 (m, |
| | | 1H), 3.37-3.32 (m, 1H), 2.67-2.62 (m, 1H), 2.36 (s, 3H), 1.96- |
| | | 1.93 (m, 1H), 1.93-1.78 (m, 1H) |
| I-268 | 418.0 | δ 10.06 (s, 1H), 9.47 (d, J = 6.8 Hz, 1H), 8.61 (s, 1H), 8.11 (d, J = |
| | | 1.2 Hz, 1H), 7.85-7.78 (m, 2H), 7.53-7.49 (m, 2H), 7.18 (t, J = |
| | | 6.8 Hz, 1H), 6, 07 (s, 2H), 4.27-4.18 (m, 3H), 4.08-4.05 (m, |
| | | 2H), 2.37 (s, 3H) |
| I-269 | 382.1 | δ 10.32 (s, 1H), 9.45 (d, J = 7.0 Hz, 1H), 8.64 (s, 1H), 7.92- |
| | | 7.79 (m, 3H), 7.59-7.52 (m, 1H), 7.44 (t, J = 8.0 Hz, 1H), 7.21 |
| | | (t, J = 6.3 Hz, 1H), 5.46-5.18 (m, 1H), 3.18-3.05 (m, 1H), |
| | | 2.05-1.90 (m, 1H), 1.62 (qd, J = 6.6, 13.3 Hz, 1H) |
| I-270 | 364.35 | δ 10.03 (s, 1H), 9.46 (d, J = 7.2 Hz, 1H), 8.60 (s, 1H), 8.10 (s, |
| | | 1H), 7.84-7.78 (m, 2H), 7.53-7.49 (m, 2H), 7.18 (t, J = 5.6 Hz, |
| | | 1H), 6.18-6.16 (m, 1H), 5.08-5.04 (m, 1H), 2.37 (s, 3H), 1.54 (d, |
| | | J = 6.8 Hz, 3H) |
| I-271 | 364.05 | δ 10.05 (s, 1H), 9.47 (d, J = 6.8 Hz, 1H), 8.61 (s, 1H), 8.10 (s, |
| | | 1H), 7.84-7.78 (m, 2H), 7.56-7.49 (m, 2H), 7.19 (t, J = 5.6 Hz, |
| | | 1H), 6.17 (bs, 1H), 5.07-5.05 (m, 1H), 2.37 (s, 3H), 1.55 (d, J = |
| | | 6.8 Hz, 3H) |
| I-272 | 397.3 | δ 9.43 (d, J = 6.8 Hz, 1H), 8.69 (d, J = 4.4 Hz, 1H), 8.40(s, |
| | | 1H), 8.29(d, J = 8.0 Hz, 1H), 8.15(d, J = 1.6 Hz, 1H), 8.03-8.00 (m, |
| | | 1H), 7.92 (dd, J1 = 8.0 Hz, J2 = 1.6 Hz, 1H), 7.65-7.59 (m, |
| | | 2H), 7.50-7.46 (m, 1H), 7.41 (d, J = 8.0 Hz, 1H), 7.08 (td, J1 = |
| | | 7.2 Hz, J2 = 1.2 Hz, 1H), 2.34 (s, 3H) |
| I-273 | 362.15 | δ 10.04 (brs, 1H), 9.46 (d, J = 7.2 Hz, 1H), 8.59 (s, 1H), 8.06 |
| | | (d, J = 1.6 Hz, 1H), 7.83-7.78 (m, 2H), 7.55-7.48 (m, 2H), 7.18 |
| | | (td, J1 = 6.8 Hz, J2 = 1.2 Hz, 1H), 3.36-3.39 (m, 1H), 2.36 (s, |
| | | 3H), 1.38 (d, J = 7.2 Hz, 6H) |
| I-274 | 552.2 | δ 9.71 (s, 1H), 9.13 (dd, J = 6.4, 1.6 Hz, 1H), 8.55 (s, 1H), 8.31 |
| | | (s, 1H), 8.01 (d, J = 1.6 Hz, 1H), 7.74 (dd, J = 8 Hz, 1.6 Hz, |
| | | 1H), 7.45 (d, J = 8 Hz, 1H), 7.12 (d, J = 8.8 Hz, 2H), 6.97 (d, J = |
| | | 9.2 Hz, 2H), 6.79-6.76 (m, 2H), 5.38-5.35 and 5.22-5.19 (dm, |
| | | J = 62.8 Hz, 1H), 3.11-3.05 (m, 5H), 2.34 (s, 3H), 1.97-1.90 (m, |
| | | 1H), 1.63-1.51 (m, 7H) |
| I-275 | 509.1 | δ 9.76 (s, 1H), 9.17(d, J = 7.6 Hz, 1H), 8.78 (s, 1H), 8.36 (s, |
| | | 1H), 8.01 (s, 1H), 7.75(dd, J = 1.6 Hz, 1H), 7.46(d, J = 8.0 Hz, |
| | | 1H), 7.2 (d, J = 8.0 Hz, 1H), 7.14 (s, 1H), 7.01 (d, J = 8.0 Hz, |
| | | 1H), 6.94(d, J = 2.4 Hz, 1H), 6.85(dd, J = 8.0 Hz, 1H), 5.38- |
| | | 5.36 and 5.22-5.19 (dm, J = 64.8 Hz, 1H), 3.07-3.04(m, 1H), |
| | | 2.88-2.84(m, 4H), 2.34 (s, 3H), 2.05-1.91 (m, 3H), 1.61-1.56(m, |
| | | 1H) |
| I-276 | 535.2 | δ 9.78 (s, 1H), 9.20 (d, J = 7.6 Hz, 1H), 8.91 (s, 1H), 8.37 (s, |
| | | 1H), 8.02 (d, J = 1.6 Hz, 1H), 7.75 (dd, J = 8 Hz, 2 Hz, 1H), |
| | | 7.46 (d, J = 8.4 Hz, 1H), 7.39-7.18 (m, 5H), 6.99 (d, J = 2 Hz, |
| | | 1H), 6.86 (dd, J = 7.6 Hz, 2.4 Hz, 1H), 5.38-5.36 and 5.22-5.19 |
| | | (dm, J = 60.8 Hz, 1H), 3.08-3.01 (m, 1H), 2.34 (s, 3H), 1.99-1.90 |
| | | (m, 1H), 1.63-1.56 (m, 1H) |
| I-277 | 461.5 | δ 9.77 (s, 1H), 9.20 (d, J = 7.6 Hz, 1H), 8.89 (s, 1H), 8.37 (s, |
| | | 1H), 8.02 (s, 1H), 7.75 (dd, J = 8.0, 1.6 Hz, 1H), 7.46 (d, J = |
| | | 8.0 Hz, 1H), 7.38 (t, J = 8.4 Hz, 2H), 7.26 (d, J = 7.6 Hz, 2H), |
| | | 7.06-7.02 (m, 2H), 6.89 (dd, J = 7.6, 2.4 Hz, 1H), 5.38-5.35 and |
| | | 5.22-5.19 (dm, J = 62.8 Hz, 1H), 3.08-3.03 (m, 1H), 2.34 (s, |
| | | 3H), 1.98-1.91 (m, 1H), 1.63-1.56(m, 1H) |
| I-278 | 469.45 | δ 9.66 (s, 1H), 9.13 (d, J = 7.6 Hz, 1H), 8.32 (s, 1H), 8.02 (s, |
| | | 1H), 7.74 (dd, J = 8.0, 1.6 Hz, 1H), 7.45 (d, J = 8.0 Hz, 1H), |
| | | 6.75 (dd, J = 7.6, 2.0 Hz, 1H), 6.44 (d, J = 2.0 Hz, 1H), 5.38- |
| | | 5.36 and 5.22-5.19 (dm, J = 62.8 Hz, 1H), 4.07-4.03 (m, 1H), |
| | | 3.46 (t, J = 8.0 Hz, 1H), 3.24-3.22 (m, 1H), 3.08-3.03 (m, 1H), |
| | | 2.34 (s, 3H), 2.08-1.97 (m, 4H), 1.72-1.71 (m, 1H), 1.61-1.56 |
| | | (m, 1H), 1.15 (d, J = 6.0 Hz, 3H) |
| I-279 | 432.4 | δ 9.64 (s, 1H), 9.01 (d, J = 7.6 Hz, 1H), 8.27 (s, 1H), 8.01 (d, J = |
| | | 1.6 Hz, 1H), 7.73 (dd, J = 8 Hz, 2 Hz, 1H), 7.44 (d, J = 8 Hz, |
| | | 1H),), 6.67-6.61 (m, 2H), 6.44 (d, J = 2 Hz, 1H), 5.38-5.35 and |
| | | 5.21-5.20 (dm, J = 66.4 Hz, 1H), 4.79 (t, J = 5.6 Hz, 1H), 3.60 |
| | | (q, J = 6 Hz, 2H), 3.18 (q, J = 6 Hz, 2H), 3.05-3.04 (m, 1H), |
| | | 2.33 (s, 3H), 1.97-1.90 (m, 1H), 1.61-1.56 (m, 1H) |
| I-280 | 510.07 | δ 9.84 (s, 1H), 8.84 (d, J = 7.2 Hz, 1H), 8.80 (s, 1H), 8.41 (d, |
| | | J = 2.0 Hz, 1 H), 8.09 (d, J = 1.6 Hz, 1H), 7.82 (dd, J1 = 7.6 Hz, |
| | | J2 = 1.2 Hz, 1H)7.48(d, J = 8.0 Hz, 1H), 7.30 (dd, J1 = 7.2 Hz, |
| | | J2 = 2.0 Hz, 1H) 4.37-4.20 (m, 5H), 3.59 (s, 3H), 2.35 (s, 3H) |
| I-281 | 494.9 | δ 10.32 (br s, 1H), 9.45 (br d, J = 6.9 Hz, 1H), 8.64 (s, 1H), |
| | | 7.99-7.85 (m, 2H), 7.79 (br d, J = 9.0 Hz, 1H), 7.54 (br t, J = |
| | | 7.8 Hz, 1H), 7.45 (br t, J = 7.9 Hz, 1H), 7.20 (br t, J = 6.8 Hz, |
| | | 1H), 4.38 (br d, J = 7.1 Hz, 2H), 4.35-4.27 (m, 1H), 4.22 (br s, |
| | | 2H), 3.60 (s, 3H) |
| I-282 | 432.40 | δ 10.04 (s, 1H), 9.46 (d, J = 6.8 Hz, 1H), 8.59 (s, 1H), 8.10 (d, J = |
| | | 1.2 Hz, 1H), 7.85-7.78 (m, 2H), 7.53-7.49 (m, 2H), 7.18 (td, J = |
| | | 6.8 Hz, 0.8 Hz, 1H), 6.45 (q, J = 4 Hz, 1H), 4.26-4.19 (m, |
| | | 3H), 4.07-4.05 (m, 2H), 2.55 (d, J = 4.8 Hz, 3H), 2.37 (s, 3H) |
| I-283 | 348.35 | δ 10.04 (brs, 1H), 9.46 (d, J = 6.8 Hz, 1H), 8.59 (s, 1H), 8.07 |
| | | (d, J = 1.2 Hz, 1H), 7.83-7.78 (m, 2H), 7.55-7.48 (m, 2H), 7.18 |
| | | (t, J = 6.8 Hz, 1H), 3.02 (q, J1 = 14.8 Hz, J2 = |
| | | 7.6 Hz, 2H), 2.37(s, 3H), 1.35(t, J = 7.6 Hz, 3H) |
| I-284 | 535.2 | δ 9.95 (s, 1H), 9.34 (d, J = 1.6 Hz, 1H), 8.49 (s, 1H), 8.34 (s, 1H), |
| | | 7.98 (d, J = 1.2 Hz, 1H), 7.77-7.70 (m, 2H), 7.48-7.46 (m, 1H), |
| | | 7.37-7.34 (m, 1H), 7.27-6.89 (m, 4H), 5.37-5.19 (m, 1H), 3.07- |
| | | 3.03 (m, 1H), 2.33 (s, 3H), 1.97-1.90 (m, 1H), 1.62-1.55 (m, 1H) |
| I-285 | 529.55 | δ 9.72 (s, 1H), 9.16 (d, J = 7.6 Hz, 1H), 8.65 (s, 1H), 8.33 (s, |
| | | 1H), 8.02 (d, J = 1.6 Hz, 1H), 7.74 (dd, J = 7.6 Hz, 1.6 Hz, |
| | | 1H)), 7.45 (d, J = 8.4 Hz, 1H), 6.98 (d, J = 8.8 Hz, 1H), 6.86- |
| | | 6.80 (m, 4H), 5.38-5.35 and 5.22-5.19 (dm, J = 62.8 Hz, 1H), |
| | | 3.76 (s, 6H), 3.09-3.01 (m, 1H), 2.34 (s, 3H), 1.98-1.89 (m, |
| | | 1H), 1.62-1.54 (m, 1H) |
| I-286 | 527.2 | δ 9.87 (s, 1H), δ 9.41 (s, 1H), 9.28 (d, J = 7.6 Hz, 1H), 8.44 (s, |
| | | 1H), 8.02 (d, J = 2 Hz, 1H), 7.95 (d, J = 8.8 Hz, 2H), 7.76 (dd, J = |
| | | 7.6 Hz, 1.6 Hz, 1H), 7.47 (d, J = 8 Hz, 1H), 7.33-7.30 (m, |
| | | 3H), 7.01 (dd, J = 7.6 Hz, 2.4 Hz, 1H), 5.38-5.35 and 5.22-5.20 |
| | | (dm, J = 61.2 Hz, 1H), 3.08-3.03 (m, 1H), 2.52 (s, 3H), 2.35 (s, |
| | | 3H), 1.98-1.92 (m, 1H), 1.63-1.54 (m, 1H) |
| I-287 | 432.15 | δ 10.04 (s, 1H), 9.46 (dd, J = 6.8 Hz, 0.8 Hz, 1H), 8.59 (s, 1H), |
| | | 8.10 (s, 1H), 7.85-7.83 (d, J = 8 Hz, 1.6 Hz, 1H), 7.79 (d, J = |
| | | 9.2 Hz, 1H), 7.53-7.49 (m, 2H), 7.18 (t, J = 6.8 Hz, 1H), 5.09 (t, |
| | | J = 5.6 Hz, 1H), 4.69-4.64 (m, 1H), 4.52-4.49 (m, 1H), 4.37- |
| | | 4.30 (m, 2H), 4.19-4.15 (m, 1H), 3.96 (s, 2H), 2.37 (s, 3H) |
| I-288 | 396.1 | δ 10.10 (s, 1H), 9.47-9.45 (m, 1H), 8.63 (s, 1H), 8.01 (d, J = |
| | | 1.2 Hz, 1H), 7.87-7.85 (m, 1H), 7.79-7.77 (m, 1H), 7.63- |
| | | 7.47 (m, 1H), 7.49 (d, J = 8.0 Hz, 1H), 5.41-5.16 (m, 1H), |
| | | 3.13-2.98 (m, 1H), 2.35 (s, 3H), 2.02-1.87 (m, 1H), 1.62- |
| | | 1.54 (m, 1H) |
| I-289 | 412.9 | δ 10.2 (s, 1H), 8.91 (d, J = 1.6 Hz, 1H), 8.53-8.51 (m, 2H), |
| | | 7.73-7.70 (m, 2 H), 7.50 (d, J = 8.4 Hz, 1H), 5.40-5.21(m, 1H), |
| | | 3.09-3.05 (m, 1H), 2.52 (s, 3H), 1.98-1.92 (m, 1H), 1.63- |
| | | 1.56 (m, 1H) |
| I-290 | 527.2 | δ 9.13-9.12 (m, 1H), 8.23 (s, 1H), 7.92 (d, J = 2.0 Hz, 1H), 7.74 |
| | | (dd, J1 = 7.6 Hz, J2 = 1.6 Hz, 1H), 7.48-7.46 (m, 1H), 7.34-7.32 |
| | | (m, 1H), 7.26 (dd, J1 = 9.6 Hz, J2 = 2.0 Hz, 1H), 6.66-6.64 (m, |
| | | 1H), 6.54-6.51 (m, 2H), 5.11-4.93 (m, 1H), 4.11-4.06 (m, 4H), |
| | | 2.75-2.70 (m, 1H), 2.27 (s, 3H), 1.83-1.75 (m, 1H), 1.54-1.48 (m, |
| | | 1H) |
| I-291 | 433.1 | δ 9.87 (s, 1H), 8.56 (d, J = 2.0 Hz, 1H), 8.44 (s, 1H), 8.00 (d, |
| | | J = 2.0 Hz, 1H), 7.75 (dd, J1 = 8.0 Hz, J2 = 1.6 Hz, 1H), 7.65 (d, |
| | | J = 9.6 Hz, 1H), 7.48 (d, J = 7.6 Hz, 1H), 7.02 (dd, J1 = 9.6 Hz, |
| | | J2 = 2.4 Hz, 1H), 5.38-5.20(m, 1H), 3.84 (t, J = 7.2 Hz, 4H), 3.09- |
| | | 3.03(m, 1H), 2.36-2.31 (m, 5H), 1.98-1.91 (m, 1H), 1.63-1.56 |
| | | (m, 1H) |
| I-292 | 497.5 | δ 9.75 (s, 1H), 9.18 (d, J = 7.6, Hz 1H), 8.74 (s, 1H), 8.36 (s, |
| | | 1H), 8.02 (d, J = 1.6, 1H), 7.75 (d, J = 1.6, 1H), 7.46 (d, J = 8.0 |
| | | Hz, 1H), 7.02 (d, J = 2.4, 1H), 6.88-6.85 (m, 3H), 6.69 (s, 1H), |
| | | 5.38-5.35 and 5.22-5.19 (dm, J = 62.8 Hz, 1H), 6.88-3.33 (m, |
| | | 1H), 3.09-3.01 (m, 1H), 2.34 (s, 3H), 2.31 (s, 6H), 1.98-1.90 |
| | | (m, 1H), 1.63-1.56 (m, 1H) |
| I-293 | 497.5 | δ 9.69 (s, 1H), 9.15(d, J = 7.2 Hz, 1H), 8.27 (s, 1H), 8.23 (s, |
| | | 1H), 8.0 (s, 1H), 7.74(dd, J = 1.6 Hz, 1H), 7.45(d, J = 8.4 Hz, |
| | | 1H), 7.22-7.15 (m, 3H), 6.73(d, J = 6.0 Hz, 1H), 5.89 (s, 1H), |
| | | 5.37-5.35 and 5.22-5.19 (dm, J = 63.2 Hz, 1H), 3.08-3.01(m, |
| | | 1H), 2.33 (s, 3H), 2.19 (s, 6H), 1.98-1.89 (m, 1H), 1.62-1.56(m, |
| | | 1H) |
| I-294 | 433.4 | δ 9.70 (s, 1H), 9.16(d, J = 7.6 Hz, 1H), 8.34 (s, 1H), 8.01 (s, |
| | | 1H), 7.73(dd, J = 1.6 Hz, 1H), 7.45(d, J = 8.0 Hz, 1H), 6.52- |
| | | 6.49 (m, 1H), 6.34 (d, J = 2.4 Hz, 1H), 5.38-5.35 and 5.22- |
| | | 5.19 (dm, J = 63.2 Hz, 1H), 3.97(t, J = 7.2 Hz, 4H), 3.07- |
| | | 3.3.03(m, 1H),), 2.40-2.33 (m, 5H), 2.34 (s, 3H), 1.97-1.91 |
| | | (m, 1H), 1.61-1.56(m, 1H) |
| I-295 | 433.4 | δ 9.70 (s, 1H), 9.16(d, J = 7.6 Hz, 1H), 8.34 (s, 1H), 8.01 (s, |
| | | 1H), 7.73(dd, J = 1.6 Hz, 1H), 7.45(d, J = 8.0 Hz, 1H), 6.52- |
| | | 6.49 (m, 1H), 6.34 (d, J = 2.4 Hz, 1H), 5.38-5.35 and 5.22- |
| | | 5.19 (dm, J = 63.2 Hz, 1H), 3.97(t, J = 7.2 Hz, 4H), 3.07- |
| | | 3.3.03(m, 1H),), 2.40-2.33 (m, 5H), 2.34 (s, 3H), 1.97-1.91 |
| | | (m, 1H), 1.61-1.56(m, 1H) |
| I-296 | 394.35 | δ 11.50 (s, 1H), 10.15 (d, J = 6.8 Hz, 1H), 8.47 (s, 1H), 7.85- |
| | | 7.80 (m, 3H), 7.59-7.52 (m, 2H), 7.21 (td, J = 6.8 Hz, 0.8 Hz, |
| | | 1H), 5.38-5.35 and 5.22-5.19 (dm, J = 65.2 Hz, 1H), 3.08-3.03 |
| | | (m, 1H), 2.32 (s, 3H), 1.98-1.90 (m, 1H), 1.63-1.54 (m, 1H) |
| I-297 | 442.1 | δ 10.07 (s, 1H), 9.47 (d, J = 6.8 Hz, 1H), 8.61 (s, 1H), 8.03 (d, J = |
| | | 1.2 Hz, 1H), 7.81-7.79 (m, 2H), 7.56 (t, J = 5.2 Hz, |
| | | 1H), 7.48(d,, J = 8.4 Hz, 1H), 7.2(dt,, J = 0.8 Hz, J = 6.8 Hz 1H), |
| | | 2.36(s, 3H), 1.99-1.94 (m, 6H), 1.50-1.46 (m, 6H), 0.84(s, 3H) |
| I-298 | 502.12 | δ = 9.99 (s, 1H), 9.12 (d, J = 6.8 Hz, 1H), 8.49-8.50 (m, 1H), |
| | | 8.05(s, 1H), 7.84(s, 1H), 7.48(d, J = 8 Hz, 1 H), 6.91(s, 2H), |
| | | 6.71(s, 1H), 3.84(m, 1H), 3.19(m, 2H), 3.07(m, 2H), 2.35(s, |
| | | 3H), 2.31(s, 3H) |
| I-299 | 497.55 | δ 9.93 (s, 1H), 9.33 (d, J = 2.0 Hz, 1H), 8.48 (s, 1H), 8.14 (s, |
| | | 1H), 8.01 (d, J = 1.6 Hz, 1H), 7.78-7.75 (m, 1H), 7.69 (d, J = 9.6 |
| | | Hz, 1H), 7.47 (d, J = 8.4 Hz, 1H), 7.39-7.36 (m, 1H), 6.66 (s, |
| | | 1H), 6.50 (s, 1H), 5.37-5.19 (m, 1H), 3.08-3.03 (m, 1H), 2.34 |
| | | (s, 3H), 2.19 (s, 6H), 1.98-1.90 (m, 1H), 1.62-1.55 (m, 1H) |
| I-300 | 469.6 | δ 9.93 (s, 1H), 9.37 (d, J = 1.6 Hz, 1H), 8.48 (s, 1H), 8.30 (s, |
| | | 1H), 7.98 (d, J = 1.6 Hz, 1 H), 7.76 (dd, J = 8.0 Hz, J = 9.6 Hz, |
| | | 1H), 7.71 (d, J = 9.6 Hz, 1H), 7.47(d, J = 8.0 Hz, 1H), 7.38(dd, |
| | | J = 2.0 Hz, J = 11.6 Hz, 1H), 7.25(t, J = 8.4 Hz, 2H), 7.06(d, |
| | | J = 7.6 Hz, 2H), 6.85(t, J = 7.6 Hz, 1 H), 5.37-5.19 (m, 1H), 3.07- |
| | | 3.03 (m, 1H), 2.33 (s, 3H), 1.96-1.90 (m, 1H), 1.62-1.54 (m, |
| | | 1H) |
| I-301 | 527.5 | δ 9.76 (s, 1H), 9.16(d, J = 7.6 Hz, 1H), 8.66 (s, 1H), 8.35 (s, |
| | | 1H), 8.02 (s, 1H), 7.75(dd, J = 1.6 Hz, 1H), 7.46(d, J = 8.0 Hz, |
| | | 1H), 6.89-6.85 (m, 2H), 6.81-6.79 (d, J = 2.4 Hz, 1H), 6.75- |
| | | 6.85 (m, 2H), 5.38-5.36 and 5.22-5.19 (dm, J = 63.2 Hz, 1H), |
| | | 4.27-4.23 (m, 2H), 3.08-3.3.03(m, 2H), 3.137(t, J = 6.0 Hz, 2H), |
| | | 2.34 (s, 3H), 1.99-1.91 (m, 1H), 1.63-1.55(m, 1H) |
| I-302 | 483.2 | δ 9.78 (s, 1H), 9.18 (d, J = 7.6 Hz, 1H), 8.81 (s, 1H), 8.36 (s, |
| | | 1H), 8.02-8.01 (m, 1H), 7.75 (dd, J = 8 Hz, 1.6 Hz, 1H), 7.46 |
| | | (d, J = 8.4 Hz, 1H), 7.21-7.15 (m, 4H), 6.95-6.94 (m, 1H), 6.85 |
| | | (dd, J = 7.6 Hz, 2.4 Hz, 1H), 5.38-5.36 and 5.22-5.19 (dm, |
| | | J = 66.8 Hz, 1H), 3.08-3.01 (m, 1H), 2.34 (s, 3H), 2.29 (s, 3H), |
| | | 1.98-1.90 (m, 1H), 1.62-1.56 (m, 1H) |
| I-303 | 474.22 | δ 9.73 (s, 1H), 9.12 (d, J = 7.6 Hz, 1H), 8.36 (s, 1H), 8.017-8.013 |
| | | (m, 1H), 7.75-7.73 (m, 1H), 7.46 (d, J = 8.0 Hz, 1H), 7.06 (dd, |
| | | J = 8.0, 2.4 Hz, 1H), 6.84-6.83 (m, 1H), 5.38-5.36 and 5.21-5.19 |
| | | (dm, J = 66.4 Hz, 1H), 3.90 (d, J = 12.8 Hz, 2H), 3.07-3.04 (m, |
| | | 1H), 2.83 (t, J = 1.2 Hz, 2H), 2.34 (s, 3H), 1.97-1.94 (m, 1H), |
| | | 1.70 (d, J = 12.4 Hz, 2H), 1.61-1.57(m, 2H), 1.23-1.18 (m, 2H), |
| | | 0.93 (d, J = 6.4 Hz, 3H) |
| I-304 | 434.19 | δ 9.64 (s, 1H), 9.01 (d, J = 7.6 Hz, 1H), 8.27 (s, 1H), 8.01 (d, J = |
| | | 1.6 Hz, 1H), 7.73 (dd, J = 8 Hz, 2 Hz, 1H), 7.44 (d, J = 8.4 |
| | | Hz, 1H), 6.64-6.61 (m, 2H), 6.39 (d, J = 2 Hz, 1H), 5.38-5.35 |
| | | and 5.22-5.19 (dm, J = 62.8 Hz, 1H), 3.09-3.03 (m, 3H), 2.35 (s, |
| | | 3H), 1.97-1.91 (m, 1H), 1.63-1.58 (m, 3H), 0.99-0.85 (m, 3H) |
| I-305 | 460.20 | δ 9.73 (s, 1H), 9.12 (d, J = 7.6, Hz 1H), 8.36 (s, 1H), 8.01 (s, |
| | | 1H), 7.74 (d, J = 7.6 Hz, 1H), 7.45 (d, J = 8.0, 1H), 7.06 (d, J = |
| | | 2.0 Hz, 1H), 6.83 (s, 1H), 2.37 (s, 3H), 7.06 (d, J = 2.0 Hz, 1H), |
| | | 5.37-5.36 and 5.21-5.20 (dm, J = 65.2 Hz, 1H), 3.39-3.33 (m, |
| | | 1H), 3.08-3.03 (m, 1H), 2.34 (s, 3H), 1.91 (m, 2H), 1.61-1.58 |
| | | (m, 7H) |
| I-306 | 364.2 | δ 10.52 (s, 1H), 9.62 (d, J = 6.8 Hz, 1H), 9.00 (s, 1H), 8.07 (d, J = |
| | | 1.6 Hz, 1H), 8.05-8.00 (m, 1H), 7.96-7.89 (m, 1H), 7.85 |
| | | (dd, J = 1.6, 7.6 Hz, 1H), 7.56-7.47 (m, 2H), 3.86 (t, J = 6.0 |
| | | Hz, 2H), 3.13 (t, J = 6.0 Hz, 2H), 2.38 (s, 3H) |
| I-307 | 465.1 | (CD₃OD, 400 MHz) δ 9.68 (d, J = 6.8 Hz, 1H), 8.77 (s, 1H), |
| | | 8.13 (s, 1H), 8.04-7.95 (m, 2H), 7.90-7.88 (m, 1H), 7.53 (t, J = |
| | | 6.8 Hz, 1H), 7.44 (d, J = 8.0 Hz, 1H), 3.03-2.95 (m, 2H), 2.88- |
| | | 2.82 (m, 4H), 2.72-2.66 (m, 2H), 2.35 (s, 3H) |
| I-308 | 434.5 | δ = 10.05 (s, 1H), 9.45 (d, J = 6.8 Hz, 1H), 8.59(s, 1H), 8.05 (s, |
| | | 1H), 7.83-7.77(m, 2H), 7.54-7.48(m, 2H), 7.18 (t, J = 6.8 |
| | | Hz, 1H), 4.07(t, J = 8.4 Hz, 1H), 3.26 (s, 3H) 3.01 (s, 3H), 2.43- |
| | | 2.38(m, 1H), 2.36 (s, 3H)2.34-2.29(m, 1H)2.22-2.13(m, 2H) |
| I-309 | 418.2 | δ 10.02 (s, 1H), 9.46 (d, J = 6.8 Hz, 1H), 8.59 (s, 1H), 8.07 (d, J = |
| | | 1.6 Hz, 1H), 7.83-7.77 (m, 2H), 7.54-7.48 (m, 2H), 7.19-7.15 |
| | | (m, 1H), 4.06-3.98 (m, 1H), 3.48-3.41 (m, 1H), 3.38 (q, J = 7.2 |
| | | Hz, 2H), 2.78-2.71 (m 2H), 2.36 (s, 3H), 2.28-2.21 (m, 2H) |
| | | 1.11 (t, J = 6.8 Hz, 3H) |
| I-310 | 418.2 | δ 10.05 (s, 1H), 9.45 (d, J = 6.8 Hz, 1H), 8.59 (s, 1H), 8.06 (d, J = |
| | | 1.2 Hz, 1H), 7.84-7.77 (m, 2H), 7.54-7.48 (m, 2H), 7.18 (t, J = |
| | | 6.8 Hz, 1H), 4.30-4.23 (m, 1H), 3.83-3.76 (m, 1H), 3.37 (q, J = |
| | | 6.8 Hz, 2H), 2.67-2.59 (m 2H), 2.47-2.42 (m, 2H), 2.36 (s, |
| | | 3H), 1.18 (t, J = 6.8 Hz, 3H) |
| I-311 | 428.3 | δ 10.52 (s, 1H), 9.46 (d, J = 7.2 Hz, 1H), 8.59 (s, 1H), 8.03 (s, |
| | | 1H), 7.81-7.77 (m, 2H), 7.49-7.47 (m, 2H), 7.19-7.16 (m, 1H), |
| | | 2.35 (s, 3H), 1.95-1.91 (m, 6H), 1.71-1.64 (m, 7H) |
| I-312 | 511.0 | δ 9.83 (s, 1H), 9.28 (dd, J1 = 1.6 Hz, J2 = 0.8 Hz, 1H), 8.79 (s, |
| | | 1H), 8.18 (d, J = 9.2 Hz, 1H), 8.10 (d, J = 1.6 Hz, 1H), 7.82- |
| | | 7.80 (m, 1H), 7.66 (dd, J 1 = 9.6 Hz, J2 = 1.6 Hz, 1H), 7.48 (d, |
| | | J = 8.0 Hz, 1H), 4.38-4.36 (m, 2H), 4.31-4.24 (m, 1H), 4.21- |
| | | 4.20 (m, 2H), 3.59 (s, 3H), 2.36 (s, 3H) |
| I-313 | 467.1 | δ 9.76 (d, J = 7.2 Hz, 1H), 8.77 (s, 1H), 8.17 (d, J = 1.6 Hz, 1H), |
| | | 8.06-8.0 (m, 2H), 7.96 (dd, J1 = 8.0 Hz, J2 = 2.0 Hz, 1H), 7.58- |
| | | 7.54 (m, 1H), 7.51 (d, J = 8.4 Hz, 1H) 4.29-4.23 (m. 1H), 3.82- |
| | | 3.78 (m, 1H), 2.56-2.46 (m, 4H), 2.45 (s, 3H) |
| I-314 | 399.4 | (CD₃OD, 400 MHz) δ 9.63 (d, J = 7.2 Hz, 1H), 8.64 (s, 1H), |
| | | 8.06 (d, J = 1.6 Hz, 1H), 7.88-7.84 (m, 3H), 7.43-7.39 (m, 2H), |
| | | 4.17-4.13 (m. 1H), 3.72-3.68 (m, 1H), 2.46-2.35 (m, 4H), 2.33 |
| | | (s, 3H) |
| I-315 | 402.0 | δ 9.40 (dd, J1 = 7.2 Hz, J2 = 0.8 Hz, 1H), 8.35 (s, 1H), 8.04- |
| | | 8.02 (m, 1H), 7.83-7.78 (m, 1H), 7.63-7.61 (m, 1H), 7.46-7.42 |
| | | (m, 1H), 7.36-7.34 (m, 1H), 7.06-7.02 (m, 1H), 3.38-3.34 (m, |
| | | 1H), 3.03-2.98 (m, 1H), 2.78-2.75 (m, 1H), 2.51-2.48 (m, 1H), |
| | | 2.35 (s, 3H), 1.86-1.78 (m, 1H), 1.14-1.05 (m, 3H), 0.98-0.95 |
| | | (m, 3H) |
| I-316 | 444.3 | δ 9.42-9.40 (m, 1H), 8.37 (s, 1H), 8.03 (d, J = 1.6 Hz, 1H), |
| | | 7.80 (dd, J1 = 8 Hz, J2 = 1.6 Hz, 1H), 7.64-7.61 (m, 1H), 7.48- |
| | | 7.44 (m, 1H), 7.36 (d, J = 8 Hz, 1H), 7.07-7.03 (m, 1H), 3.65- |
| | | 3.61 (m, 1H), 3.35 (s, 3H), 3.14-3.09 (m, 1H), 2.48-2.42 (m, |
| | | 1H), 2.30 (s, 3H), 2.25-2.08 (m 4H), 1.73-1.61 (m, 2H), 1.47 |
| | | (m, 1H) |
| I-317 | 444.3 | δ 9.42(d, J = 6.8 Hz, 1H), 8.38(s, 1H), 8.02(d, J = 1.6 Hz, 1H), 7.81 (dd, |
| | | J1 = 8 Hz, J2 = 1.6 Hz 1H), 7.64 (d, J = 9.2 Hz 1H), 7.50-7.46 |
| | | (m, 1H), 7.37 (d, J = 8.4 Hz, 1H), 7.09-7.06 (m, 1H), 3.99 (t, |
| | | 7.4 Hz, 1H), 3.46 (dd, J1 = 8 Hz, J2 = 3.4 Hz 1H), 3.31 (s, 3H), |
| | | 2.58-2.50 (m, 2H), 2.31 (s, 3H), 2.15-2.12 (m, 1H), 1.94-1.77 |
| | | (m, 4H), 1.70-1.64 (m, 1H) |
| I-318 | 406.2 | δ 9.42 (d, J = 6.8 Hz, 1H), 8.38 (s, 1H), 8.01 (d, J = 1.6 Hz, |
| | | 1H), 7.79 (dd, J1 = 8 Hz, J2 = 2 Hz 1H), 7.65-7.63 (m, 1H), |
| | | 7.49-7.45 (m, 1H), 7.38-7.36 (m, 1H), 7.09-7.05 (m, 1H), 5.27- |
| | | 5.12 (m, 1H), 3.68-3.59 (m 1H), 2.39-2.31 (m, 2H), 2.30 (s, |
| | | 3H), 2.19-1.95 (m, 4H). |
| I-319 | 406.4 | (CD₃OD, 400 MHz) δ 9.63-9.61 (d, J = 8 Hz, 1H), 8.67 (s, 1H), |
| | | 8.37 (d, J = 2 Hz, 1H), 7.88-7.83 (m, J = 20 Hz, 1H), 7.61-7.59 (d, |
| | | J = 8 Hz, 2H), 7.44-7.40 (q, J = 16 Hz, 1H), 5.13-4.74 (m, J = 156 |
| | | Hz, 1H), 2.80-2.74 (m, J = 24 Hz, 1H), 1.85-1.76 (m, J = 36 |
| | | Hz, 1H), 1.56-1.49 (m, J = 28 Hz, 1H) |
| I-320 | 440.45 | δ 9.63 (d, J = 7.2 Hz, 1H), 8.64 (s, 1H), 8.06 (d, J = 1.6 Hz 1H), |
| | | 7.88-7.84 (m, 3H), 7.43-7.39 (m, 2H), 3.92 (s, 2H), 3.13-2.84 |
| | | (m, 4H), 2.33 (s, 3H) |
| I-321 | 392.45 | δ 10.02 (s, 1H), 9.46 (d, J = 6.8 Hz, 1H), 8.59 (s, 1H), 8.10 (d, J = |
| | | 1.6 Hz, 1H), 7.83-7.78 (m, 2H), 7.53-7.50 (m, 2H), 7.18 (t, J = |
| | | 6.8 Hz, 0.8 Hz, 1H), 4.39 (s, 2H), 3.71 (s, 3H), 2.37 (s, 3H) |
| I-322 | 468.2 | δ 10.02 (s, 1H), 9.46 (d, J = 7.2 Hz, 1H), 8.59 (s, 1H), 8.10 (d, J = |
| | | 1.2 Hz, 1H), 7.83-7.78 (m, 2H), 7.54-7.50 (m, 2H), 7.39-7.29 |
| | | (m, 5H), 7.18 (t, J = 6.8 Hz, 0.8 Hz, 1H), 5.21 (s, 2H), 4.46(s, |
| | | 2H), 2.37(s, 3H) |
| I-323 | 448.55 | δ 9.70 (d, J = 6.8 Hz, 1H), 8.70 (s, 1H), 8.10 (s, 1H), 7.96-7.94 |
| | | (m, 2H), 7.91-7.88 (m, 1H), 7.49-7.46 (m, 2H), 2.59 (s, 2H), |
| | | 2.40 (s, 3H), 2.32 (s, 1H), 2.17 (d, J = 11.2 Hz, 2H), 1.70 (d, J = |
| | | 11.6 Hz, 2H) |
| I-324 | 458.0 | δ 9.43-9.41 (m, 1H), 8.38 (s, 1H), 8.01 (d, J = 1.6 Hz, 1H), |
| | | 7.80-7.78 (m, 1H), 7.65-7.63 (m, 1H), 7.49-7.45 (m, 1H), 7.36 |
| | | (d, J = 8.0 Hz, 1H), 7.08-7.05 (m, 1H), 3.12 (s, 3H), 2.60-2.57 |
| | | (m, 2H), 2.41-2.37 (m, 2H), 2.31 (s, 3H), 1.96-1.93 (m, 2H), |
| | | 1.80-1.69 (m, 6H) |
| I-325 | 454.55 | δ 10.05 (s, 1H), 9.46 (d, J = 6.8 Hz, 1H), 8.59 (s, 1H), 8.08 (s, |
| | | 1H), 7.85-7.78 (m, 2H), 7.54-7.49 (m, 2H), 7.18 (t, J = 6.4 Hz, |
| | | 1H), 6.31 (t, J = 54.8 Hz, 1H), 3.68-3.58 (m, 1H), 3.34 (s, 3H), |
| | | 2.78-2.62 (m, 4H), 2.37 (s, 3H) |
| I-326 | 417.5 | δ 9.42 (d, 1H), 8.38 (s, 1H), 8.04 (d, J = 7.2 Hz, 1H), 7.82 (dd, |
| | | J1 = 2 Hz, J2 = 8 Hz, 1H), 7.64 (m, 1H), 7.48 (m, 1H), 7.38 (d, J = |
| | | 8 Hz 1H), 7.07 (m, 1H), 5.05 (q, J1 = 3.2 Hz, J2 = 5.6 Hz, 1H), |
| | | 2.80 (s, 3H), 2.53 (m, 2H), 2.38 (m, 1H), 2.32 (s, 3H) 2.22 (m, |
| | | 1H) |
| I-327 | 403.45 | δ9.82(d, J = 6.8 Hz, 1H), 8.88(d, J = 3.2 Hz, 1H), 8.18-8.14(m, 2H), |
| | | 8.08 (d, J = 9.2 Hz, 1 H), 7.96 (dd, J = 1.6 Hz, 8.0 Hz, 1H), 7.67 |
| | | (t, J = 6.8 Hz, 1H), 7.51(d, J = 8.0 Hz, 1 H), 5.17-5.14 (m, 1 H), |
| | | 2.75-2.68 (m, 1H), 2.60-2.52 (m, 1H), 2.48-2.38 (s, 5H) |
| I-328 | 439.05, | δ 10.07 (s, 1H), 9.45 (d, J = 6.8 Hz, 1H), 8.60 (s, 1H), 8.05 (s, |
| | | 1H), 7.77-7.83 (m, 2H), 7.49-7.54 (m, 2H), 7.18 (t, J = 6.8 Hz, |
| | | 1H), 3.77-3.80 (m, 1H), 3.37-3.42 (m, 1H), 3.21-3.28 (m, 1H), |
| | | 2.98-3.02 (m, 1H), 2.36 (s, 3H), 1.66 (s, 3H) |
| I-329 | 453.4 | δ 10.03 (s 1H), 9.45 (d J = 8 Hz, 1H), 8.58 (s 1H), 8.0 (d |
| | | J = 1.6 Hz, 1H), 7.79-7.76 (m 2H), 7.52-7.47 (m, 2H), 7.19- |
| | | 7.16 (t, 1H), 5.32-5.17 (dd J = 4 Hz 1H), 2.39-2.30 (m, 6H), |
| | | 1.93-1.95 (m, 4H) |
| I-330 | 439.1 | δ 10.04 (s, 1H), 9.46 (d, J = 8 Hz, 1H), 8.59 (s, 1H), 8.09 (s, |
| | | 1H), 8.01 (s, 1H), 7.80 (m, 2H), 7.53 (t, J = 8 Hz 2H), 7.18 (t, J = |
| | | 7.1 Hz 1H), 5.11 (t, J = 8 Hz, 1H), 3.37-3.43 (m, 1H) 3.15-3.22 |
| | | (m, 1H), 2.90-2.94 (m, 1H), 2.71-2.77 (m, 1H), 2.33 (s, 3H) |
| I-331 | 453.4 | δ 9.63 (d, J = 7.2 Hz, 1H), 8.64 (s, 1H), 8.07 (d, J = 1.6 Hz 1H), |
| | | 7.88-7.84 (m, 3H), 7.43-7.40 (m, 2H), 5.55-5.51 (m, 1H), 4.13- |
| | | 4.07 (m, 1H), 3.85-3.80 (m, 1H), 2.96(s, 3H), 2.67-2.62 (m, |
| | | 2H), 2.33 (s, 3H) |
| I-332 | 540.95 | 10.221 (s, 1H), 9.461-9.444 (d, J = 6.8 Hz, 1H), 8.626 (s, 1H), |
| | | 8.257-8.252 (d, J = 2.0 Hz, 1H), 7.970-7.949 (d, J = 8.4 Hz, 1H), |
| | | 7.873-7.846 (dd, J1 = 2.0 Hz, J2 = 2.4 Hz, 1H), 7.815-7.793 (d, |
| | | J = 8.8 Hz, 1H), 7.568-7.528 (t, 1H), 7.217-7.182 (t, 1H), 4.331- |
| | | 4.230 (m, 3H), 4.130 (bs, 2H), 1.40 (s, 9H) |
| I-333 | 495.5 | δ 10.24 (s, 1H), 9.46-9.44 (d, J = 8 Hz, 1H), 8.63 (s, 1H), 8.32- |
| | | 8.31 (d, J = 4 Hz 1H), 7.94-7.92 (m, J = 8 Hz, 1H), 7.81-7.78 |
| | | (m, J = 1 Hz, 2H), 7.57-7.52 (t, J = 20 Hz, 1H), 7.22-7.18 (t, J = 16 |
| | | Hz, 1H) 4.33-4.24 (m, J = 36 Hz, 3H), 4.13 (brs, 2H), 1.40 (s, 9H) |
| I-334 | 462.2 | δ 10.06 (s, 1H), 9.45 (d, J = 1.2 Hz, 1H), 8.58 (s, 1H), 8.02 (s, |
| | | 1H), 7.79-7.77 (m, 2H), 7.54-7.48 (m, 2H), 7.18 (t, J = 7.2 Hz, |
| | | 1H), 2.69-2.67 (m, 1H), 2.35 (s, 3H), 2.317-2.30(m, 2H), 2.19- |
| | | 2.16(m, 1H), 2.03-2.01(m, 2H), 1.86-1.83 (m, 2H), 1.72- |
| | | 1.70(m, 1H) |
| I-335 | 378.0 | δ 10.04 (s, 1H), 9.47 (d, J = 6.8 Hz, 1H), 8.61 (s, 1H), 8.10 (d, J = |
| | | 0.6 Hz, 1H), 7.83-7.78 (m, 2H), 7.54-7.49 (m, 2H), 7.18 (t, J = |
| | | 6.8 Hz, 1H), 4.24 (s, 2H), 2.37 (s, 3H) |
| I-336 | 392.45 | δ 10.03 (s, 1H), 9.46 (d, J = 6.8 Hz, 1H), 8.59 (s, 1H), 8.07 (s, |
| | | 1H), 7.83-7.77 (m, 2H), 7.54-7.48 (m, 2H), 7.18 (t, J = 6.8 Hz, |
| | | 1H), 4.87 (s, 1H), 3.09 (s, 2H), 2.36 (s, 3H), 1.27 (s, 6H). |
| I-337 | 350.4 | δ 9.60 (d, J = 6.8 Hz, 1H), 8.61 (s, 1H), 8.05 (s, 1H), 7.85-7.82 |
| | | (m, 3H), 7.41-7.35 (m, 2H), 4.75 (d, J = 7.2 Hz, 2H), 2.32 (s, |
| | | 3H) |
| I-338 | 498.95 | 10.221 (s, 1H), 9.458-9.441 (d, J = 6.8 Hz, 1H), 8.626 (s, 1H), |
| | | 8.251-8.252 (d, J = 2.0 Hz, 1H), 7.968-7.947 (d, J = 8.4 Hz, 1H), |
| | | 7.873-7.87 (d, J1 = 1.2 Hz, 1H), 7.852-7.793 (m, 2H), 7.566- |
| | | 7.527 (t, 1H), 7.215-7.181 (t, 1H), 4.38-4.225 (m, 5H), 3.594 |
| | | (s, 3H) |
| I-339 | 453.2 | δ 10.24 (s, 1H), 9.46 (d, J = 6.8 Hz, 1H), 8.64 (s, 1H), 8.33 (d, J = |
| | | 2.0 Hz, 1H), 7.94 (m 1H), 7.85-7.73 (m, 2H), 7.60-7.50 (m, |
| | | 1H), 7.21 (t, J = 6.8 Hz, 1H), 4.38 (d, J = 7.6 Hz, 2H), 4.31 (m |
| | | 1H), 4.22 (d, J = 4.8 Hz, 2H), 3.60 (s, 3H) |
| I-340 | 443.9 | 10.221 (s, 1H), 9.45-9.43 (d, J = 8.0 Hz, 1H), 8.624 (s, 1H), |
| | | 8.175-8.17 (d, J = 2.0 Hz, 1H), 8.002-7.981 (d, J = 8.4 Hz, 1H), |
| | | 7.817-7.786 (m, 2H), 7.569-7.53 (t, 1H), 7.22-7.183 (t, 1H), |
| | | 5.392-5.206 (m, 1H), 3.114-3.058 (m, 1H), 1.998-1.924 (m, |
| | | 1H), 1.644-1.561 (s, 1H) |
| I-341 | 397.07 | δ 10.25 (s, 1H), 9.44 (d, J = 6.8 Hz, 1H), 8.62 (s, 1H), 8.23 (d, |
| | | J = 1.6 Hz, 1H) 7.88-7.85 (m, 1H), 7.81-7.77 (m, 2H), 7.57-7.53 |
| | | (m,, 1H), 7.22-7.18 (m, 1H), 5.39-5.20 (m, 1H), 3.10-3.07 |
| | | (m, 1H), 1.99-1.93 (m, 1H), 1.62-1.57 (m, 1H) |
| I-342 | 432.2 | δ 10.07 (s, 1H), 9.45 (d, J = 6.8 Hz, 1H), 8.60 (s, 1H), 8.05 (s, |
| | | 1H), 7.77-7.83 (m, 2H), 7.49-7.54 (m, 2H), 7.18 (t, J = 6.8 Hz, |
| | | 1H), 3.77-3.80 (m, 1H), 3.37-3.42 (m, 1H), 3.21-3.28 (m, 1H), |
| | | 2.98-3.02 (m, 1H), 2.36 (s, 3H), 1.66 (s, 3H) |
| I-343 | 446.6 | δ = 10.04 (d, J = 8.8 Hz, 1H), 9.46 (dd, J = 2.8 Hz, 7.2 Hz, 1H), |
| | | 8.59 (s, 1H), 8.06 (d, J = 11.2 Hz, 1 H), 7.83-7.77 (m, 2 H), |
| | | 7.54-7.48 (m, 2 H), 7.18 (t, J = 6.8 Hz, 1 H), 3.95-3.90 (m, 0.5 |
| | | H), 3.66-3.55 (m, 6.5 H), 2.67-2.54 (m, 2H), 2.36 (s, 3H), 2.33- |
| | | 2.30 (m, 2H) |
| I-344 | 416.5 | δ 10.03 (S, 1H), 9.45 (d, J = 8 Hz, 1H), 8.58 (S, 1H), 8.07(d, J = |
| | | 1.6 Hz, 1H), 7.83-7.77 (m, 2H), 7.54-7.58 (m, 2H), 7.20-7.16 |
| | | (m, 1H), 4.05 (s, 2H), 2.33 (s, 3H), 2.30-2.29 (m, 2H), 1.95(dd, |
| | | J1 = 4.0 Hz, J2 = 4.4 Hz, 2H), 1.44 (s, 3H) |
| I-345 | 430.55 | δ = 10.03 (s, 1H), 9.45 (d, J = 6.8 Hz, 1 H), 8.59 (s, 1H), 8.06 |
| | | (d, J = 1.2 Hz, 1 H), 7.82-7.77 (m, 2 H), 7.54-7.48 (m, 2 H), |
| | | 7.18 (t, J = 7.2 Hz, 1H), 4.10 (t, J = 6.4 Hz, 2H), 2.45-2.39 (m, 4 |
| | | H), 2.33 (s, 3H), 2.34-2.38 (m, 2H), 1.26 (s, 3H) |
| I-346 | 434.55 | δ = 10.04 (s, 1H), 9.46 (d, J = 7.2 Hz, 1H), 8.59(s, 1H), 8.07 (s, |
| | | 1H), 7.83-7.77(m, 2H), 7.54-7.48(m, 2H), 7.19-7.16 (t, J = 6.8 |
| | | Hz, 1H), 3.65-3.61(m, 1H), 3.12-3 (s, 3H) 3.08 (s, 3H), 2.74- |
| | | 2.67(m, 2H), 2.36 (s, 3H) |
| I-347 | 466.65 | δ 10.04 (s, 1H), 9.46 (d, J = 6.8, Hz, 1H), 8.62 (s, 1H), 8.12 (s, |
| | | 1H), 7.82 (dd, J = 1.6, J = 9.2 Hz, 2H), 7.54-7.50 (m, 2H), 7.29 |
| | | (t, J = 7.6 Hz, 2H), 7.18 (t, J = 6.8 Hz, 1H), 6.97 (t, J = 7.2 Hz, |
| | | 1H), 6.87 (d, J = 8 Hz, 2H), 5.06-5.0 (m, 1H), 4.02-3.95 (m 1H), |
| | | 2.95-2.91 (m, 2H), 2.71-2.66 (m, 2H), 2.37 (s, 3H) |
| I-348 | 466.7 | δ 10.03 (s, 1H), 9.46 (d, J = 7.2, Hz, 1H), 8.59 (s, 1H), 8.08 (s, |
| | | 1H), 7.80 (dd, J = 8.0, J = 8.8 Hz, 2H), 7.54-7.48 (m, 2H), 7.29 |
| | | (t, J = 7.6 Hz, 2H), 7.18 (t, J = 6.8 Hz, 1H), 6.96-6.88 (m, 3H), |
| | | 4.83 (t, J = 7.2 Hz 1H), 3.64 (t. J = 9.6 Hz 1H), 3.06 (d, J = 9.2 |
| | | Hz, 2H), 2.34 (s, 3H) |
| I-349 | 466.0 | δ 10.04 (s, 1H), 9.46 (d, J = 6.8 Hz, 1H), 8.60 (s, 1H), 8.10 (d, |
| | | J = 1.2 Hz, 1H), 7.84 (dd, J1 = 8.0 Hz, J2 = 1.6 Hz, 1H), 7.79 (d, |
| | | J = 8.8 Hz, 1H), 7.61-7.54 (m, 2H), 7.53-7.49 (m, 2H), 7.41- |
| | | 7.38 (m, 2H), 7.31-7.29 (m, 1H), 7.22-7.16 (m, 1H), 5.91(s, |
| | | 1H), 3.59-3.53 (m, 1H), 2.99-2.96 (m, 2H), 2.81-2.76 (m, 2H), |
| | | 2.37 (s, 3H) |
| I-350 | 404.5 | δ 10.05-10.03 (m, 1H), 9.46 (d, J = 6.8 Hz, 1H), 8.59 (s, 1H), |
| | | 8.08-8.06 (m, 1H), 7.82-7.78 (m, 2H), 7.55-7.48 (m, 2H), 7.18 |
| | | (t, J = 6.8 Hz, 0.8 Hz, 1H), 3.98-3.91 (m, 1H), 3.49-3.45 (m, |
| | | 1H), 3.18 (s, 3H), 2.78-2.72 (m, 2H), 2.69-2.58 (m, 1H), 2.38- |
| | | 2.34 (m, 3H), 2.26-2.23(m, 1H) |
| I-351 | 450.6 | δ = 10.06 (s, 1H), 9.46 (d, J = 6.8 Hz, 1 H), 8.59 (s, 1H), 8.07 |
| | | (d, J = 1.2 Hz, 1 H), 7.85-7.77 (m, 2 H), 7.54-7.50 (m, 2 H), |
| | | 7.19-7.16 (m, 1 H), 2.54-2.51 (m, 1 H), 2.37 (s, 3H), 2.28-2.13 |
| | | (m, 3H), 2.07-2.00 (m, 4H) 1.61-1.52 (m, 1H) |
| I-352 | 436.5 | δ 10.05 (s, 1H), 9.45 (d, J = 6.8 Hz, 1H), 8.58 (s, 1H), 8.03 (d, J = |
| | | 1.2 Hz, 1H), 7.81-7.77 (m, 2H), 7.54-7.48 (m, 2H), 7.18 (t, J = |
| | | 6.8 Hz, 1H), 2.60-2.59 (m, 1H), 2.36 (s, 3H), 2.22-2.13 (m, |
| | | 3H), 1.95-1.93(m, 1H), 1.87-1.84 (m, 1H), 1.70-1.65 (m, 1H) |
| I-353 | 486.5 | 10.04(d, J = 9.6 Hz, 1H), 9.45-9.48(m, 1H), 8.59(d, J = 4.4 Hz, |
| | | 1H), 8.07(s, 1H), 7.78-7.81(m, 2H), 7.47-7.52(m, 4H), 7.36(d, |
| | | J = 8.4 Hz, 1H), 7.18-7.20(m, 2H), 4.23-4.31(m, 1H), 3.10- |
| | | 3.16(m, 4H), 2.36(s, 3H) |
| I-354 | 470.5 | δ 10.06 (s, 1H), 9.47 (d, J = 7.2 Hz, 1H), 8.59 (s, 1H), 8.07 (s, |
| | | 1H), 7.83-7.78 (m, 2H), 7.55-7.50 (m, 2H), 7.18 (t, J = 8.0 Hz, |
| | | 1H), 3.39-3.35 (m, 2H), 3.29-3.13 (m, 2H), 3.09-3.03 (m, 2H), |
| | | 2.37(s, 3H), 2.00 (s, 3H) |
| I-355 | 460.45 | δ 9.70 (d, J = 7.2 Hz, 1H), 8.73 (s, 1H), 8.09 (d, J = 1.2 Hz, 1H), |
| | | 8.02-7.94 (m, 2H), 7.88 (dd, J1 = 8.0 Hz, J2 = 1.2 Hz |
| | | 1H), 7.52(t,, J = 6.8 Hz,, 1H), 7.42 (d, J = 8.4 Hz, 1H), 6.58-6.30 |
| | | (m, 1H), 3.26-3.24 (m, 4H), 2.34 (s, 3H) |
| | 453.45 | δ 9.60 (d, J = 6.8 Hz, 1H), 8.59 (s, 1H), 8.07 (d, J = 1.6 Hz 1H), |
| | | 7.87-7.81 (m, 3H), 7.42-7.34 (m, 2H), 5.55-5.51 (m, 1H), 4.12- |
| | | 4.07 (m, 1H), 3.85-3.81 (m, 1H), 2.96(s, 3H), 2.67-2.62 (m, |
| | | 2H), 2.33 (s, 3H) |
| I-357 | 460.55 | δ 10.03 (s, 1H), 9.46 (d, J = 6.8 Hz, 1H), 8.59 (s, 1H), 8.07 (d, J = |
| | | 1.2 Hz, 1H), 7.83-7.77 (m, 2H), 7.54-7.48 (m, 2H), 7.18 (t, J = |
| | | 6.8 Hz, 1H), 5.30 (s, 1H), 5.17 (s, 1H), 3.41-3.37 (m, 1H), |
| | | 2.72-2.61 (m, 2H), 2.36 (s, 3H), 2.33-2.28 (m, 4H), 1.85-1.81 |
| | | (m, 2H), 1.71-1.69 (m, 1H), 1.52-1.50 (m, 1H) |
| I-358 | 401.0 | δ 9.78-9.77 (d, J = 4 Hz, 1H), 8.86 (s, 1H), 8.23-8.22 (d, J = |
| | | 4 Hz, 1H), 8.13-8.05 (m, 2H), 7.99-7.96 (dd, J = 12 Hz, 1H), |
| | | 7.64-7.60 (t, 1H), 7.54-7.52 (d, J = 8 Hz, 1H), 5.01-4.98 (m, |
| | | J = 12 Hz, 1H) 3.59-3.54 (t, 1H) 2.85-2.80 (m, 1H) 2.68-2.64 (m, |
| | | 1H) 2.45 (s, 3H) 2.10-2.06 (m, 1H),) 1.26-1.22 (m, J = 16 Hz, |
| | | 1H) 1.06-1.02 (m, 1H) |
| I-359 | 424.8 | (CD₃OD, 400 MHz) δ 10.74 (s, 1H), 9.63 (d, J = 7.2 Hz, 1H), |
| | | 9.18 (s, 1H), 8.11-8.04 (m, 2H), 8.00-7.94 (m, 1H), 7.91- |
| | | 7.86 (m, 1H), 7.58-7.52 (m, 2H), 5.43 (t, J = 8.8 Hz, 1H), 3.86- |
| | | 3.80 (m, 2H), 3.19-3.11 (m, 1H), 3.05-3.01 (m, 1H), 2.41 (s, |
| | | 3H) |
| I-360 | 334.3 | (CD₃OD, 400 MHz) δ 10.02 (s, 1H), 9.46 (d, J = 6.8 Hz, 1H), |
| | | 8.59 (s, 1H), 8.08 (s, 1H), 7.81-7.78 (m, 2H), 7.54-7.48 (m, |
| | | 2H), 7.18 (t, J = 6.0 Hz, 1H), 2.67 (s, 3H), 2.36 (s, 3H) |
| I-361 | 447.5 | δ 9.81 (s, 1H), 8.68 (d, J = 2.0 Hz, 1H), 8.43 (s, 1H), 8.02 |
| | | (s, 1H), 7.77-7.75 (m, 1H), 7.63 (d, J = 9.6 Hz, 1H), 7.47 (d, J = |
| | | 8.0 Hz, 1H), 7.24-7.21 (m, 1H), 5.38-5.20 (m, 1H), 3.23 (t, J = |
| | | 6.4 Hz, 4H), 3.08-3.03 (m, 1H), 2.35 (s, 3H), 1.99-1.91 (m, |
| | | 5H), 1.63-1.56 (m, 1H) |
| I-362 | 418.5 | δ = 10.04 (s, 1H), 9.46 (d, J = 6.8 Hz, 1H), 8.60 (s, 1H), 8.06 (s, |
| | | 1H), 7.82-7.78 (t, 2H), 7.55-7.48(m, 2H), 7.20-7.17 (t, J = 6.8 |
| | | Hz, 1H), 3.84-3.71 (m, 2H), 3.36-3.16 (m, 2H), 2.99-2.86(m, |
| | | 2H), 2.36 (s, 3H), 2.10 (s, 1H), 1.85-1.82(d, J = 10 Hz 1H), 1.60- |
| | | 1.23 (m, 3H) |
| I-363 | 378.4 | (CD₃OD, 400 MHz) δ 10.06 (s, 1H), 9.47 (d, J = 6.8 Hz, 1H), |
| | | 8.62 (s, 1H), 8.08 (s, 1H), 7.83-7.79 (m, 2H), 7.56-7.48 (m, |
| | | 2H), 7.20 (t, J = 5.6 Hz, 1H), 5.05 (bs, 1H), 4.21-4.13 (m, 1H), |
| | | 3.12-2.99 (m, 2H), 2.37 (s, 3H), 1.30-1.09 (m, 3H) |
| I-364 | 364.4 | δ 10.03 (s, 1H), 9.46 (d, J = 6.8 Hz, 1H), 8.59 (s, 1H), 8.09 (s, |
| | | 1H), 7.84-7.78 (m, 2H), 7.54-7.49 (m, 2H), 7.18 (t, J = 6.8 Hz, |
| | | 1H), 6.16 (d, J = 5.6 Hz, 1H), 5.07-5.04 (m, 1H), 2.37 (s, 3H), |
| | | 1.55-1.53 (m, 3H) |
| I-365 | 447.6 | δ 9.65 (s, 1H), 9.14 (d, J = 7.6 Hz, 1H), 8.33 (s, 1H), 8.02 (d, J = |
| | | 1.6 Hz, 1H), 7.73 (dd,, J = 7.6 Hz, 1.6 Hz, 1H), 7.45 (d, J = |
| | | 8.4 Hz, 1H), 6.73 (dd, J = 7.6 Hz, 2.4 Hz, 1H), 6.44 (d, J = 2 |
| | | Hz, 1H), 5.38-5.20 (m, 1H), 3.40-3.34 (m, 4H), 3.07-3.03 (m, |
| | | 1H), 2.34-2.32 (m, 3H), 2.01-1.94 (m, 5H), 1.61-1.56 (m, 1H) |
| I-366 | 432.1 | δ 9.63 (d, J = 6.8 Hz, 1H), 8.64 (s, 1H), 8.04 (d, J = 1.6 Hz, |
| | | 1H), 7.88-7.87 (m, 2H), 7.82 (dd, J1 = 8.0 Hz, J2 = 1.6 Hz, |
| | | 1H), 7.43-7.37 (m, 2H), 3.76-3.72 (m, 1H), 3.59 (s, 3H), 3.25- |
| | | 3.19 (m, 1H), 2.64-2.59 (m, 4H), 2.32 (s, 3H) |
| I-367 | 414.3 | δ 10.09 (s, 1H), 9.47 (d, J = 6.8 Hz, 1H), 8.62 (s, 1H), 8.08(d, J = |
| | | 1.2, 1H), 7.86-7.79 (m, 2H), 7.56-7.49 (m, 2H), 7.21-7.18 (m, |
| | | 1H), 3.72 (t, J = 8.2, 1H), 2.37 (s, 3H), 2.34-2.27 (m, 2H), 2.15- |
| | | 2.11 (m, 3H), 2.04-2.02 (m, 1H), 1.82-1.67 (m, 4H) |
| I-368 | 416.5 | δ 10.02 (s, 1H), 9.45 (d, J = 7.2 Hz, 1H), 8.59 (s, 1H), 8.07 (s, |
| | | 1H), 7.81-7.78 (m, 2H), 7.45-7.48 (m, 2H), 7.20-7.16 (m, 1H), |
| | | 3.28 (s, 3H), 2.42 (s, 6H), 2.36 (s, 3H) |
| I-369 | 436.5 | (CD₃OD, 400 MHz) δ 9.52 (d, J = 7.2 Hz, 1H), 8.49 (s, 1H), |
| | | 8.12 (d, J = 1.6 Hz, 1H), 7.91 (d, J = 8 Hz, 1H), 7.74 (d, J = 8.8 |
| | | Hz, 1H), 7.60-7.56 (m, 1H), 7.47 (d, J = 8 Hz, 1H), 7.19-7.16 |
| | | (m, 1H), 4.59-4.57 (m, 1H), 4.47-4.42 (m, 2H), 3.80-3.77 (m, |
| | | 1H), 3.70-3.68 (m, 1H), 3.62-3.60 (m, 1H), 2.74-2.69 (m, 2H), |
| | | 2.60-2.57 (m, 2H), 2.42 (s, 3H) |
| I-370 | 385.4 | (CD₃OD, 400 MHz) δ 10.84 (s, 1H), 9.65 (d, J = 7.2 Hz, 1H), |
| | | 9.26 (s, 1H), 8.11-8.06 (m, 2H), 8.03-7.97 (m, 1H), 7.88 (dd, |
| | | J = 1.6, 8.0 Hz, 1H), 7.60-7.54 (m, 2H), 5.46 (t, J = 8.8 Hz, |
| | | 1H), 3.93-3.78 (m, 2H), 3.23-3.12 (m, 1H), 3.09-2.98 (m, |
| | | 1H), 2.41 (s, 3H) |
| I-371 | 503.3 | δ 10.80 (bs, 1H), 10.347 (s, 1H), 10.0 (bs, 1H), 9.530-9.513 (d, |
| | | J = 6.8 Hz, 1H), 8.846 (s, 1H), 8.115-8.111 (d, J = 1.6 Hz, 1H), |
| | | 7.910-7.856 (m, 2H), 7.718-7.679 (t, 1H), 7.557-7.537 (d, J = 8.0 |
| | | Hz, 1H), 7.341-7.307 (t, 1H), 5.301-5.280 (t, 1H), 3.141 (s, |
| | | 2H), 2.395 (s, 3H), 2.371-2.252 (m, 2H), 1.206-1.193 (d, 6H) |
| I-372 | 417.5 | δ 10.03 (bs, 1H), 9.46 (d, J = 6.8 Hz, 1H), 8.59 (s, 1H), 8.07 (s, |
| | | 1H), 7.80 (t, J = 9.6 Hz, 2H), 7.54-7.48 (m, 2H), 7.18 (td, J = |
| | | 6.8 Hz, 1.2 Hz, 1H), 4.17-4.14 (m, 1H), 3.26-3.14 (m, 4H), 2.36 |
| | | (s, 3H), 2.0-2.01 (m, 1H), 1.81-1.79 (m, 3H), 1.36 (s, 3H), 1.30 |
| | | (s, 6H) |
| I-373 | 385.4 | δ 10.03 (s, 1H), 9.46-9.44 (m, 1H), 8.59 (s, 1H), 8.03 (d, J = 1.2 |
| | | Hz, 1H), 7.80-7.77 (m, 2H), 7.54-7.48 (m, 2H), 7.19-7.16 (m, |
| | | 1H), 3.37-3.32 (m, 1H), 2.67-2.62 (m, 1H), 2.36 (s, 3H), 1.96- |
| | | 1.93 (m, 1H), 1.93-1.78 (m, 1H) |
| I-374 | 438.5 | δ 10.08 (s, 1H), 9.46 (d, J = 7.2 Hz, 1 H), 8.60 (s, 1H), 8.06 (d, |
| | | J = 1.2 Hz, 1 H), 7.85-7.78 (m, 2 H), 7.56-7.50 (m, 2 H), 7.19 (t, |
| | | J = 6.8 Hz, 1 H), 2.70-2.62 (m, 1 H), 2.47-2.38 (m, 1H), 2.36 (s, |
| | | 3H), 2.33-2.28 (m, 1H), 2.10-2.06 (m, 1H), 2.00-1.94 (m, 1H), |
| | | 1.57 (d, J = 1.6 Hz, 3H) |
| I-375 | 469.9 | δ 10.06 (s, 1H), 9.45 (d, J = 7.2 Hz, 1H), 8.59 (s, 1H), 8.05 (d, J = |
| | | 1.6 Hz, 1H), 7.83-7.78 (m, 2H), 7.53-7.50 (m, 2H), 7.18 (t, J = |
| | | 7.2 Hz, 1H), 2.87-.82 (m, 2H), 2.70-2.66 (m, 1H), 2.36 (s, |
| | | 3H), 2.02-1.99 (m, 1H), 1.79 (br s, 1H) |
| I-376 | 392.5 | (CD₃OD, 400 MHz) δ 0.037 (s, 1H), 9.45 (d, J = 6.8 Hz, 1H), |
| | | 8.59 (s, 1H), 8.07 (d, J = 1.6 Hz, 1H), 7.84-7.78 (m, 2H), 7.55- |
| | | 7.49 (m, 2H), 7.18 (dt, J = 6.8 Hz, 0.8 Hz, 1H), 5.47-5.3(m, |
| | | 1H), 3.98-3.94 (m, 1H), 2.32-2.40 (m, 4H),, 2.31-2.22 (m, 2H), |
| | | 1.83-1.88 (m, 1H) |
| I-377 | 392.5 | (CD₃OD, 400 MHz) δ 10.06 (s, 1H), 9.46 (d, J = 6.8 Hz, 1H), |
| | | 8.6 (s, 1H), 8.09 (d, J = 1.6 Hz, 1H), 7.86-7.78 (m, 2H), 7.55- |
| | | 7.50 (m, 2H), 7.18 (dt, J = 6.8 Hz, 0.8 Hz, 1H), 5.54-5.35(m, |
| | | 1H), 4.32-4.26 (m, 1H), 2.55-2.47 (m, 3H),, 2.37 (s, 3H), 2.18- |
| | | 2.13 (m, 1H) |
| I-378 | 454.8 | δ 10.07 (s, 1H), 9.46 (d, J = 6.8 Hz, 1H), 8.60 (s, 1H), 8.08(s, |
| | | 1H), 7.85-7.78 (m, 2H), 7.55-7.50 (m, 2H), 7.20-7.16 (m, 1H), |
| | | 3.86 (s, 2H), 3.31-3.21 (m, 5H, merged with DMSO-d₆ |
| | | moisture), 3.10-3.00 (m, 2H), 2.37 (s, 3H) |
| I-379 | 475.3 | δ 10.04 (s, 1H), 9.46 (d, J = 6.8 Hz, 1H), 8.59 (s, 1H), 8.10 (m, |
| | | J = 1.6 Hz, 1H), 8.83 (dd, J = 1.2, J = 9.2 Hz, 2H), 7.53 (t, J = |
| | | 1.2, 2H), 7.18 (t, J = 7.2 Hz, 1H), 5.51 (t, J = 5.6, 1H), 4.06- |
| | | 3.96 (m, 2H), 2.70-2.66 (m, 2H), 2.33 (s. 3H), 1.35 (s, 9H) |
| I-380 | 426.2 | δ 10.06 (s, 1H), 9.46-9.45 (d, J = 5.6 Hz, 1H), 8.59 (s, 1H), 8.09 |
| | | (s, 1H), 7.85-7.78 (dd, 2H), 7.52-7.50 (d, J = 7.2 Hz, 2H), 7.16 |
| | | (s, 2H), 3.47-3.33 (m, 2H), 3.04-3.02 (m, 2H), 2.33 (s, 3H) |
| I-381 | 392.2 | (CD₃OD, 400 MHz) δ 10.03 (s, 1H), 9.46 (d, J = 6.8 Hz, 1H), |
| | | 8.59 (s, 1H), 8.08 (s, 1H), 7.80 (dd, J = 8.4, 15.6 Hz, 2H), 7.57- |
| | | 7.45 (m, 2H), 7.17 (t, J = 6.8 Hz, 1H), 5.29-5.01 (m, 1H), 3.53- |
| | | 3.38 (m, 1H), 2.99-2.82 (m, 2H), 2.70-2.53 (m, 2H), 2.37 (s, |
| | | 3H |
| I-382 | 456.1 | δ 10.13 (s, 1H), 9.62-9.61 (m, 1H), 8.59 (s, 1H), 8.01(d, J = 1.2 |
| | | Hz, 1H), 7.78 (dd, J1 = 8.0 Hz, J2 = 4.0 Hz, 1H), 5.37-5.20 |
| | | (m, 1H), 3.07-3.06(S, 1H), 2.35-2.32 (s, 3H), 1.99-1.91(m, |
| | | 1H)), 1.61-1.56(m, 1H) |
| I-383 | 456.1 | δ 10.10 (s, 1H), 9.35 (d, J = 8.0 Hz, 1H), 8.57 (s, 1H), 8.13 (d, |
| | | J = 1.6 Hz, 1H), 8.00(d J = 1.2 Hz, 1H), 7.78(dd, J1 = 7.6 Hz, J2 = 1.6 |
| | | Hz 1H), 7.49 (d, J = 8.0 Hz, 1H), 7.36 (dd, J1 = 7.6 Hz, J2 = 2.0 Hz, |
| | | 1H)), 5.26(m, 1H), 2.35-2.33 (s, 3H), 1.61-1.56 (m, 1H), 1.23 |
| | | (m, 1H) |
| I-384 | 403.1 | δ 10.25 (s, 1H), 9.93 (s, 1H), 8.70 (s, 1H), 8.02 (s, 1H), 7.96 (d, |
| | | J = 9.2 Hz, 1H), 7.81-7.79 (m, 2H), 7.50 (d, J = 8 Hz, 1H), 5.38- |
| | | 5.21 (m, 1H), 3.09-3.03 (m, 1H), 2.35 (s, 3H), 1.98-1.92 (m, |
| | | 1H)), 1.62-1.56 (m, 1H) |
| I-385 | 424.2 | δ 10.05 (s, 1H), 9.465-9.450 (d, J = 6 Hz, 1H), 8.593 (s, 1H), 8.069 |
| | | (s, 1H), 7.777-7.844 (m, 2H), 7.498-7.516 (t, 2H), 7.180-7.196 (t, 1H), |
| | | 3.257-3.334 (m, 2H), 2.850-2.936 (m, 2H), 2.303-2.366 (s, 3H), |
| | | 1.718 (s, 3H) |
| I-386 | 406.2 | δ 10.06 (brs, 1H), 9.46 (d, J = 6.8 Hz, 1H), 8.59 (s, 1H), 8.05 |
| | | (d, J = 1.6 Hz, 1H), 7.84-7.78 (m, 2H), 7.55-7.49 (m, 2H), 7.20- |
| | | 7.16 (m, 1H), 5.38-5.21 (m, 1H), 3.06-2.98 (m, 2H), 2.47-2.42 |
| | | (m, 2H), 2.36 (s, 3H), 1.70 (s, 3H) |
| I-387 | 406.1 | (CD₃OD, 400 MHz) δ 9.42 (d, J = 7.2 Hz, 1H), 8.38 (s, 1H), |
| | | 8.21 (d, J = 1.6 Hz, 1H), 7.81(dd, J = 2.0 Hz, J = 8.0 Hz, 1H), 7.62(d, |
| | | J = 9.2 Hz, 1H), 7.50-7.36 (m, 1H), 7.37 (d, J = 8.0 Hz 1H), |
| | | 7.09-7.05 (m, 1H), 3.88-3.79 (m, 1H), 2.80-2.72 (m, 2H), 2.59- |
| | | 2.53 (m, 2H), 2.31(s, 3H), 1.42(d, J = 22.0 Hz, 3H) |
| I-388 | 503.2 | δ 10.05 (s, 1H), 9.45 (d, J = 6.8 Hz, 1H), 8.59 (s, 1H), 8.07 (s, |
| | | 1H), 7.84-7.78 (m, 2H), 7.54-7.49 (m, 2H), 7.19-7.16 (m, 1H), |
| | | 3.42-3.38 (m, 2H), 3.27-3.26 (m, 3H), 3.15-3.07 (m, 1H), 3.01- |
| | | 2.90 (m, 2H), 2.36 (s, 3H), 1.96-1.91 (m, 1H) |
| I-389 | 438.2 | δ 10.05 (brs, 1H), 9.45 (d, J = 6.4 Hz, 1H), 8.59 (s, 1H), 8.07 (s, |
| | | 1H), 7.84-7.77 (m, 2H), 7.54-7.50 (m, 2H), 7.19-7.16 (m, 1H), |
| | | 4.24-4.20 (m, 1H), 3.74-3.68 (m, 1H), 3.52-3.46 (m, 1H), 3.30- |
| | | 3.25 (m, 2H), 2.72-2.67 (m, 2H), 2.36 (s, 3H) |
| I-390 | 452.1 | δ 9.53 (d, J = 6.8 Hz, 1H), 8.48 (s, 1H), 8.13 (s, 1H), 7.91 (dd, |
| | | J1 = 8.0 Hz, J2 = 1.6 Hz, 1H), 7.74 (d, J = 9.2 Hz, 1H), 7.59- |
| | | 7.56 (m, 1H), 7.48 (d, J = 8.0 Hz, 1H), 7.19-7.16 (m, 1H), 4.05- |
| | | 3.90 (m, 1H), 3.76-3.73 (m, 2H), 3.47-3.43 (m, 1H), 3.15-3.07 |
| | | (m, 1H), 2.70-2.60 (m, 1H), 2.42 (s, 3H), 1.5 (s, 9H), 1.23-1.20 |
| | | (m, 3H) |
| I-391 | 389.1 | δ 9.66 (d, J = 7.2 Hz, 1H), 8.73 (s, 1H), 8.13 (s, 1H), 7.97-7.87 |
| | | (m, 3H), 7.5 (t, J = 1.6 Hz, 1H), 7.45 (d, J = 7.6 Hz, 1H), 5.1(t, J = |
| | | 7.6 Hz, 1H), 3.52-3.43 (m, 2H), 2.61-2.56 (m, 1H), 2.37-2.34 |
| | | (m, 3H), 2.33-2.31 (m, 1H), 2.20-2.15 (m, 2H) |
| I-392 | 404.1 | δ 10.03 (brs, 1H), 9.46 (d, J = 6.8 Hz, 1H), 8.60 (s, 1H), 8.09 (s, |
| | | 1H), 7.82-7.78 (m, 2H), 7.55-7.48 (m, 2H), 7.20-7.17 (m, 1H), |
| | | 2.69 (s, 6H), 2.37 (s, 3H) |
| I-393 | 375.1 | δ 10.04 (s, 1H), 9.46 (m, 1H), 8.59 (s, 1H), 8.07 (d, J = 1.6 Hz, |
| | | 1H), 7.87-7.71 (m, 2H), 7.56-7.44 (m, 2H), 7.18 (m, 1H), |
| | | 5.50-5.27 (m, 1H), 4.07-3.79 (m, 1H), 2.82-2.78 (m, 2H), |
| | | 2.78-2.69 (m, 2H), 2.36 (s, 3H) |
| I-394 | 375.1 | (400 MHz, CD₃OD) δ 9.68 (d, J = 6.8 Hz, 1H), 8.71 (s, 1H), 8.24 |
| | | (d, J = 1.6 Hz, 1H), 8.01-7.90 (m, 3H), 7.53 (d, J = 8 Hz, 1H), |
| | | 7.47-7.43 (m, 1H), 5.94 (t, J = 8.8 Hz, 1H),), 4.33-4.28 (m, 1H), |
| | | 4.21-4.15 (m, 1H), 3.16-3.04 (m, 2H), 2.45 (s, 3H) |
| I-395 | 475.2 | δ 9.86 (brs, 1H), 9.47 (d, J = 6.8 Hz, 1H), 8.61 (s, 1H), 8.12 (s, |
| | | 1H), 7.83-7.78 (m, 2H), 7.60-7.56 (m, 1H), 7.48 (d, J = 8.0 Hz, |
| | | 1H), 7.23-7.19 (m, 1H), 5.49-5.45 (m, 1H), 4.06-3.93 (m, 2H), |
| | | 2.75-2.70 (m, 2H), 2.38 (s, 3H), 1.25 (s, 9H) |
| I-396 | 390.0 | δ 10.15 (s, 1H), 9.51 (d, J = 6.8 Hz, 1H), 8.68 (s, 1H), 8.08 (d, J = |
| | | 1.6 Hz, 1H), 7.87-7.82 (m, 2H), 7.68-7.64 (m, 1H), 7.51 (d, J = |
| | | 8.0 Hz, 1H), 7.31-7.27 (m, 1H), 5.32-5.28 (m, 1H), 3.96-3.87 |
| | | (m, 3H), 2.39 (s, 3H), 2.33-2.32 (m, 1H), 2.05-1.99 (m, 2H) |
| I-397 | 404.2 | δ 9.72 (d, J = 7.2 Hz, 1H), 8.72 (s, 1H), 8.14 (s, 1H), 7.96-7.95 |
| | | (m, 3H), 7.51-7.47 (m, 2H), 4.84 (d, J = 2.8 Hz, 1H), 4.10-4.07 |
| | | (m, 1H), 3.73-3.67 (m, 1H), 2.42 (s, 3H), 2.07-2.01 (m, 1H), |
| | | 1.97-1.93 (m, 2H), 1.74-1.66 (m, 3H) |
| I-398 | 489 | δ 10.04 (s, 1H), 9.46 (d, J = 6.8 Hz, 1H), 8.59 (s, 1H), 8.06 (d, J = |
| | | 1.6 Hz, 1H), 7.83-7.78 (m, 2H), 7.54-7.48 (m, 2H), 7.20-7.16 |
| | | (m, 1H), 3.91-3.89 (m, 1H), 3.76-3.74 (m, 1H), 3.63-3.60 (m, |
| | | 1H), 3.47-3.37 (m, 3H), 2.36 (m, 3H), 2.23-2.20 (m, 1H), 1.26 |
| | | (s, 9H) |
| I-399 | 489.2 | δ 9.47 (d, J = 6.8 Hz, 1H), 8.45 (s, 1H), 8.10 (s, 1H), 7.83 (d, J = |
| | | 8 Hz, 1H), 7.70 (d, J = 8.8 Hz 1H), 7.55-7.51 (m, 1H), 7.40 (d, |
| | | J = 8 Hz 1H), 7.13-7.10 (m, 1H), 3.80-3.70 (m, 3H), 3.56- |
| | | 3.44(m, 2H), 2.44-2.27 (s, 5H), 1.42 (s, 9H) |
| I-1 | 436.2 | δ 9.67 (s, 1H), 8.66 (s, 1H), 8.58 (s, 1H), 8.11 (d, J = 9.6 Hz, |
| | | 1H), 8.03 (s, 1H), 7.74 (d, J = 7.6 Hz, 1H), 7.45 (d, J = 8.0 Hz, |
| | | 1H), 7.34-7.29 (m, 1H), 5.39-5.18 (m, 1H), 4.73-4.65 |
| | | (m, 1H), 3.10-3.00 (m, 1H), 2.34 (s, 3H), 2.00-1.87 (m, 1H), |
| | | 1.64-1.53 (m, 1H), 1.31 (d, J = 6.0 Hz, 6H) |
| I-2 | 460.3 | δ 10.25 (d, J = 10.0 Hz, 2H), 8.71 (s, 1H), 8.07-7.96 (m, 3H), |
| | | 7.86-7.76 (m, 1H), 7.51 (d, J = 8.0 Hz, 1H), 5.43-5.18 (m, |
| | | 1H), 3.15-2.98 (m, 1H), 2.44 (s, 3H), 2.37 (s, 3H), 1.99-1.90 |
| | | (m, 1H), 1.65-1.54 (m, 1H) |
| I-3 | 475.2 | δ 10.11 (s, 1H), 9.46 (dd, J = 0.9, 7.1 Hz, 1H), 8.66 (s, 1H), |
| | | 8.03 (d, J = 1.6 Hz, 1H), 7.96-7.89 (m, 1H), 7.79 (dd, J = 1.7, |
| | | 7.9 Hz, 1H), 7.50 (d, J = 8.1 Hz, 1H), 7.29 (dd, J = 1.7, 7.2 Hz, |
| | | 1H), 5.48-5.10 (m, 1H), 3.51 (t, J = 6.4 Hz, 4H), 3.06 (dddd, J = |
| | | 1.8, 6.9, 11.1, 16.6 Hz, 1H), 2.37 (s, 3H), 2.03-1.78 (m, 5H), |
| | | 1.59 (qd, J = 6.7, 13.1 Hz, 1H) |
| I-4 | 461.1 | δ 10.01 (s, 1H), 9.82 (dd, J = 0.8, 2.0 Hz, 1H), 8.58 (s, 1H), |
| | | 8.02 (d, J = 1.6 Hz, 1H), 7.93 (dd, J = 2.0, 9.6 Hz, 1H), 7.83- |
| | | 7.76 (m, 2H), 7.48 (d, J = 8.0 Hz, 1H), 5.38-5.19 (m, 1H), |
| | | 3.90-3.86 (m, 2H), 3.10-3.03 (m, 1H), 2.54-2.52 (m, 2H), |
| | | 2.35 (s, 3H), 2.14-2.08 (m, 2H), 1.99-1.89 (m, 1H), 1.61- |
| | | 1.56 (m, 1H) |
| I-5 | 511.1 | δ 0.89-0.96 (m, 4 H), 1.59 (m, 1H), 1.88-2.03 (m, 1H), 2.37 |
| | | (s, 3H), 2.97-3.15 (m, 1H), 4.30 (d, J = 4.4 Hz, 2H), 5.13- |
| | | 5.45 (m, 1H), 7.45-7.57 (m, 2H), 7.74-7.86 (m, 3H), 8.05 (s, |
| | | 1H), 8.58 (s, 1H), 9.47 (s, 1H), 10.03 (s, 1H) |
| I-6 | 480.3 | δ 10.05 (s, 1H), 9.41 (d, J = 7.1 Hz, 1H), 8.59 (s, 1H), 8.15 (d, J = |
| | | 1.2 Hz, 1H), 7.90-7.85 (m, 1H), 7.74 (s, 1H), 7.56 (d, J = 8.0 |
| | | Hz, 1H), 7.20-7.10 (m, 1H), 5.25-5.01 (m, 1H), 4.66 (s, 2H), |
| | | 4.56-4.34 (m, 1H), 3.28 (s, 2H), 2.85-2.72 (m, 1H), 2.40 (s, |
| | | 3H), 1.85-1.72 (m, 1H), 1.45-1.34 (m, 1H), 1.15 (s, 6H) |
| I-7 | 465.1 | δ 1.55-1.64 (m, 1H), 1.89-2.01 (m, 1H), 2.36 (s, 3H), 2.99- |
| | | 3.15 (m, 1H), 3.92 (s, 2H), 4.43 (d, J = 6.0 Hz, 2H), 5.19-5.40 |
| | | (m, 1H), 5.51-5.71 (m, 1H), 7.07-7.14 (m, 1H), 7.49 (d, J = |
| | | 7.2 Hz, 1H), 7.56-7.61 (m, 1H), 7.78 (m, 1H), 8.03 (d, J = 1.6 |
| | | Hz, 1H), 8.47-8.54, (m, 1H), 8.57 (s, 1H), 9.38 (d, J = 7.2 Hz, |
| | | 1H), 10.01 (s, 1H) |
| I-8 | 465.3 | δ 10.01 (s, 1H), 9.38 (s, 1H), 8.57 (s, 1H), 8.52-8.43 (m, 1H), |
| | | 8.03 (d, J = 1.6 Hz, 1H), 7.80-7.73 (m, 2H), 7.52-7.47 (m, |
| | | 2H), 5.38-5.19 (m, 1H), 4.36 (d, J = 6.0 Hz, 2H), 3.84 (s, 2H), |
| | | 3.06 (s, 1H), 2.35 (s, 3H), 1.99-1.90 (m, 1H), 1.64-1.54 (m, |
| | | 1H) |
| I-9 | 449.3 | δ 10.01 (s, 1H), 9.36 (d, J = 0.8 Hz, 1H), 8.56 (s, 1H), 8.47 (t, J = |
| | | 5.6 Hz, 1H), 8.02 (d, J = 1.6 Hz, 1H), 7.79-7.73 (m, 2H), |
| | | 7.50-7.41 (m, 2H), 5.39-5.18 (m, 1H), 4.32 (d, J = 6.0 Hz, |
| | | 2H), 3.11-3.01 (m, 1H), 2.35 (s, 3H), 2.01-1.90 (m, 1H), 1.86 |
| | | (s, 3H), 1.64-1.55 (m, 1H) |
| I-10 | 511.2 | δ 10.01 (s, 1H), 9.40 (d, J = 7.2 Hz, 1H), 8.56 (s, 1H), 8.02 (d, J = |
| | | 1.6 Hz, 1H), 7.87 (t, J = 6.4 Hz, 1H), 7.81-7.75 (m, 1H), |
| | | 7.72 (s, 1H), 7.48 (d, J = 8.0 Hz, 1H), 7.21-7.15 (m, 1H), 5.40- |
| | | 5.16 (m, 1H), 4.34 (d, J = 6.4 Hz, 2H), 3.11-3.01 (m, 1H), |
| | | 2.58-2.53 (m, 1H), 2.35 (s, 3H), 1.94 (s, 1H), 1.62-1.53 (m, |
| | | 1H), 0.95-0.87 (m, 4H) |
| I-11 | 475.2 | δ 10.07-9.99 (m, 1H), 9.49-9.41 (m, 1H), 8.64-8.55 (m, 1H), |
| | | 8.04 (s, 1H), 7.82-7.75 (m, 2H), 7.71 (d, J = 3.6 Hz, 1H), 7.53- |
| | | 7.47 (m, 2H), 5.42-5.14 (m, 1H), 4.31-4.20 (m, 2H), 3.11- |
| | | 3.04 (m, 1H), 2.95 (s, 3H), 2.37 (s, 3H), 2.01-1.92 (m, 1H), |
| | | 1.65-1.52 (m, 1H) |
| I-12 | 470.4 | δ 10.05 (s, 1H), 9.57 (s, 1H), 8.61 (s, 1H), 8.06 (s, 1H), 7.89- |
| | | 7.71 (m, 2H), 7.58-7.38 (m, 2H), 5.44-5.13 (m, 1H), 4.67 (s, |
| | | 2H), 3.04 (d, J = 11.2 Hz, 1H), 2.99 (s, 3H), 2.37 (s, 3H), 2.02- |
| | | 1.85 (m, 1H), 1.69-1.53 (m, 1H) |
| I-13 | 498.2 | δ 10.12 (s, 1H), 9.53 (d, J = 7.8 Hz, 1H), 8.64 (s, 1H), 8.08 (d, J = |
| | | 1.2 Hz, 1H), 7.91-7.77 (m, 2H), 7.51 (d, J = 8.0 Hz, 1H), |
| | | 7.27 (dd, J = 1.6, 7.6 Hz, 1H), 6.23-5.89 (m, 2H), 4.34-4.14 |
| | | (m, 1H), 3.28 (d, J = 4.0 Hz, 1H), 3.20-3.09 (m, 1H), 2.37 (s, |
| | | 3H) |
| I-14 | 461.4 | δ 9.95 (s, 1H), 9.36 (d, J = 8.4 Hz, 1H), 8.53 (s, 1H), 8.02 (d, J = |
| | | 2.0 Hz, 1H), 7.86 (dd, J = 2.4, 7.6 Hz, 1H), 7.82-7.74 (m, |
| | | 2H), 7.48 (d, J = 8.0 Hz, 1H), 5.44-5.15 (m, 1H), 3.94 (t, J = |
| | | 7.2 Hz, 2H), 3.10-3.00 (m, 1H), 2.61-2.57 (m, 2H), 2.35 (s, |
| | | 3H), 2.15-2.07 (m, 2H), 2.00-1.88 (m, 1H), 1.69-1.47 (m, |
| | | 1H) |
| I-15 | 417.1 | δ 10.06 (s, 1H), 9.54 (s, 1H), 8.59 (s, 1H), 8.04 (d, J = 1.6 Hz, |
| | | 1H), 7.83 (d, J = 9.2 Hz, 1H), 7.78 (dd, J = 1.6, 8.0 Hz, 1H), |
| | | 7.52-7.46 (m, 2H), 5.39-5.18 (m, 1H), 4.20 (s, 2H), 3.12- |
| | | 3.00 (m, 1H), 2.36 (s, 3H), 2.01-1.87 (m, 1H), 1.64-1.53 (m, |
| | | 1H) |
| I-16 | 417.3 | δ 10.06 (s, 1H), 9.44 (d, J = 6.8 Hz, 1H), 8.59 (s, 1H), 8.02 (d, J = |
| | | 1.2 Hz, 1H), 7.80-7.72 (m, 2H), 7.48 (d, J = 8.4 Hz, 1H), |
| | | 7.19-7.13 (m, 1H), 5.39-5.18 (m, 1H), 4.23 (s, 2H), 3.11- |
| | | 3.01 (m, 1H), 2.35 (s, 3H), 1.99-1.89 (m, 1H), 1.63-1.54 (m, |
| | | 1H) |
| I-17 | 484.9 | δ10.0 (s, 1H), 9.40 (d, J = 7.2 Hz, 1H), 8.56 (s, 1H), 8.02 (d, J = |
| | | 1.6 Hz, 1H), 7.80-7.75 (m, 2H), 7.70 (s, 1H), 7.48 (d, J = 8.0 |
| | | Hz, 1H), 7.15 (dd, J = 1.6, 7.2 Hz, 1H), 5.39-5.18 (m, 1H), |
| | | 4.30 (d, J = 6.4 Hz, 2H), 3.10-3.01 (m, 1H), 2.96 (s, 3H), 2.35 |
| | | (s, 3H), 2.01-1.88 (m, 1H), 1.58 (qd, J = 6.8, 13.2 Hz, 1H) |
| I-18 | 456.1 | δ 10.25 (s, 1H), 10.01 (d, J = 1.2 Hz, 1H), 8.73 (s, 1H), 8.04- |
| | | 7.98 (m, 2H), 7.91 (dd, J = 2.0, 9.6 Hz, 1H), 7.80 (dd, J = 1.6, |
| | | 8.0 Hz, 1H), 7.50 (d, J = 8.0 Hz, 1H), 5.41-5.17 (m, 1H), 3.36 |
| | | (s, 3H), 3.12-3.00 (m, 1H), 2.37 (s, 3H), 2.02-1.87 (m, 1H), |
| | | 1.64-1.53 (m, 1H) |
| I-19 | 462.2 | δ 10.28-10.05 (m, 1H), 9.53 (d, J = 7.8 Hz, 1H), 8.65 (s, 1H), |
| | | 8.14 (d, J = 2.0 Hz, 1H), 7.92-7.85 (m, 2H), 7.56 (d, J = 8.0 |
| | | Hz, 1H), 7.32-7.24 (m, 1H), 5.24-5.02 (m, 1H), 2.85-2.73 |
| | | (m, 1H), 2.40 (s, 3H), 1.84-1.72 (m, 1H), 1.45-1.33 (m, 1H) |
| I-20 | 426.2 | δ 9.88 (s, 1H), 9.22 (s, 1H), 8.89 (s, 1H), 8.36 (d, J = 9.2 Hz, |
| | | 1H), 8.13 (d, J = 1.6 Hz, 1H), 7.87-7.76 (m, 1H), 7.68 (d, J = |
| | | 9.2 Hz, 1H), 7.50 (d, J = 8.0 Hz, 1H), 7.37-6.98 (m, 1H), 5.07- |
| | | 4.92 (m, 2H), 4.85 (t, J = 6.4 Hz, 2H), 4.76-4.64 (m, 1H), |
| | | 2.37 (s, 3H) |
| I-21 | 407.9 | δ 9.70 (s, 1H), 8.67 (s, 1H), 8.55 (d, J = 2.0 Hz, 1H), 8.12 (d, J = |
| | | 9.6 Hz, 1H), 8.03 (d, J = 1.6 Hz, 1H), 7.76-7.72 (m, 1H), |
| | | 7.45 (d, J = 8.0 Hz, 1H), 7.34-7.30 (m, 1H), 5.39-5.18 (m, |
| | | 1H), 3.87 (s, 3H), 3.10-2.99 (m, 1H), 2.34 (s, 3H), 2.00-1.87 |
| | | (m, 1H), 1.64-1.53 (m, 1H) |
| I-22 | 452.2 | δ 10.0 (s, 1H), 9.40 (d, J = 7.2 Hz, 1H), 8.57 (s, 1H), 8.06 (d, J = |
| | | 1.2 Hz, 1H), 7.80 (dd, J = 2.0, 8.0 Hz, 1H), 7.72 (s, 1H), 7.49 |
| | | (d, J = 8.0 Hz, 1H), 7.13 (dd, J = 1.2, 7.2 Hz, 1H), 5.38-5.10 |
| | | (m, 1H), 4.72 (t, J = 5.2 Hz, 1H), 4.63 (s, 2H), 3.63-3.56 (m, |
| | | 2H), 3.56-3.52 (m, 2H), 2.75-2.64 (m, 1H), 2.36 (s, 3H), 1.96- |
| | | 1.83 (m, 1H), 1.68-1.56 (m, 1H) |
| I-23 | 452.2 | δ 10.61 (s, 1H), 9.58 (d, J = 7.2 Hz, 1H), 9.07 (s, 1H), 8.04- |
| | | 7.93 (m, 2H), 7.86-7.78 (m, 1H), 7.54-7.47 (m, 2H), 5.38- |
| | | 5.20 (m, 1H), 4.78 (s, 2H), 3.64-3.59 (m, 4H), 3.11-3.02 (m, |
| | | 1H), 2.39 (s, 3H), 2.02-1.88 (m, 1H), 1.65-1.51 (m, 1H) |
| I-24 | 460.1 | δ 10.14 (s, 1H), 9.55 (d, J = 7.6 Hz, 1H), 8.65 (s, 1H), 8.19- |
| | | 8.08 (m, 1H), 7.91-7.82 (m, 2H), 7.53 (d, J = 7.6 Hz, 1H), |
| | | 7.32-7.24 (m, 1H), 5.01-4.93 (m, 2H), 4.83 (s, 2H), 4.77- |
| | | 4.67 (m, 1H), 2.38 (s, 3H) |
| I-25 | 384 | (400 MHz, CDCl₃) δ 9.39 (d, J = 7.6 Hz, 1H), 9.20 (d, J = 1.6 |
| | | Hz, 1H), 8.25 (s, 1H), 8.18 (s, 1H), 7.80 (dd, J = 1.6, 8.4 Hz, |
| | | 1H), 7.55 (d, J = 8.4 Hz, 1H), 7.09-7.03 (m, 1H), 6.78 (dd, J = |
| | | 2.4, 7.6 Hz, 1H), 3.94 (s, 3H), 2.68 (s, 3H) |
| I-26 | 417.2 | (400 MHz, CDCl₃) δ 1.64 (br d, J = 6.4 Hz, 1H), 1.79-1.93 (m, |
| | | 1H), 2.42 (s, 3H), 2.57 (s, 3H), 2.68-2.80 (m, 1H), 4.99 (td, J = |
| | | 4.0, 1.71 Hz, 1H), 5.15 (td, J = 4.0, 1.71 Hz, 1H), 7.39 (d, J = |
| | | 7.6 Hz, 1H), 7.65 (s, 1H), 7.85 (d, J = 7.6 Hz, 1H), 8.10 (s, 1H), |
| | | 8.28 (s, 1H), 8.48 (s, 1H), 9.51 (s, 1H) |
| I-27 | 426.2 | δ 1.58 (dd, J = 13.2, 6.32 Hz, 1H), 1.88-2.01 (m, 1H), 2.34 (s, |
| | | 3H), 3.01-3.11 (m, 1H), 4.00 (s, 3H), 5.20 (ddd, J = 6.00, 3.75, |
| | | 1.75 Hz, 1H), 5.36 (ddd, J = 6.00, 3.75, 1.88 Hz, 1H), 7.40 (d, J |
| | | 8.4 Hz, 1H), 7.47 (d, J = 8.00 Hz, 1H), 7.77 (dd, J = 7.6, 1.75 = |
| | | Hz, 1H), 8.00 (d, J = 1.2 Hz, 1H), 8.49 (s, 1H), 9.40 (d, J = 6.8 |
| | | Hz, 1H), 9.98 (s, 1H) |
| I-28 | 420.2 | δ 9.99 (s, 1H), 9.44 (s, 1H), 8.56 (s, 1H), 8.08-8.00 (m, 1H), |
| | | 7.81-7.75 (m, 1H), 7.70 (d, J = 0.8 Hz, 1H), 7.49 (d, J = 8.0 |
| | | Hz, 1H), 5.39-5.19 (m, 1H), 5.06 (d, J = 6.0 Hz, 4H), 3.12- |
| | | 3.01 (m, 1H), 2.36 (s, 3H), 2.03-1.89 (m, 1H), 1.64-1.54 (m, |
| | | 1H) |
| I-29 | 458.3 | δ 10.02 (s, 1H), 9.60 (s, 1H), 8.53 (s, 1H), 8.02 (d, J = 1.8 Hz, |
| | | 1H), 7.86-7.73 (m, 1H), 7.49 (d, J = 8.3 Hz, 1H), 7.39-7.04 |
| | | (m, 2H), 5.43-5.17 (m, 1H), 3.99 (s, 3H), 3.12-3.00 (m, 1H), |
| | | 2.36 (s, 3H), 2.03-1.86 (m, 1H), 1.67-1.50 (m, 1H) |
| I-30 | 465.2 | δ 9.98 (s, 1H), 9.37 (d, J = 7.6 Hz, 1H), 8.55 (d, J = 1.2 Hz, |
| | | 1H), 8.06-7.98 (m, 1H), 7.83 (t, J = 6.0 Hz, 1H), 7.79-7.74 |
| | | (m, 1H), 7.55 (s, 1H), 7.48 (d, J = 8.0 Hz, 1H), 7.15-7.02 (m, |
| | | 1H), 5.44-5.14 (m, 1H), 4.31 (d, J = 6.0 Hz, 2H), 3.59 (s, 3H), |
| | | 3.12-2.99 (m, 1H), 2.37-2.36 (m, 1H), 2.35 (s, 2H), 2.03- |
| | | 1.86 (m, 1H), 1.66-1.52 (m, 1H) |
| I-31 | 493.3 | δ 9.96 (s, 1H), 9.36 (d, J = 7.2 Hz, 1H), 8.54 (s, 1H), 8.42 (t, J = |
| | | 6.4 Hz, 1H), 8.07-7.96 (m, 1H), 7.80-7.72 (m, 1H), 7.53 (s, |
| | | 1H), 7.48 (d, J = 8.0 Hz, 1H), 7.11-7.02 (m, 1H), 5.48 (s, 1H), |
| | | 5.44-5.14 (m, 1H), 4.37 (d, J = 6.0 Hz, 2H), 3.14-2.97 (m, |
| | | 1H), 2.35 (s, 3H), 2.02-1.87 (m, 1H), 1.69-1.48 (m, 1H), 1.30 |
| | | (s, 6H) |
| I-32 | 449.3 | δ 10.08 (s, 1H), 9.41 (d, J = 7.2 Hz, 1H), 8.61 (s, 1H), 8.53 (t, J = |
| | | 6.0 Hz, 1H), 8.02 (d, J = 1.6 Hz, 1H), 7.78 (dd, J = 1.6, 8.0 |
| | | Hz, 1H), 7.61 (s, 1H), 7.49 (d, J = 8.0 Hz, 1H), 7.15 (d, J = 7.2 |
| | | Hz, 1H), 5.40-5.16 (m, 1H), 4.40 (d, J = 6.0 Hz, 2H), 3.10- |
| | | 3.00 (m, 1H), 2.35 (s, 3H), 2.01-1.88 (m, 4H), 1.58 (qd, J = |
| | | 6.8, 13.2 Hz, 1H) |
| I-33 | 505.3 | δ 9.98 (s, 1H), 9.36 (d, J = 7.2 Hz, 1H), 8.59-8.49 (m, 2H), |
| | | 8.05-8.00 (m, 1H), 7.77 (dd, J = 1.6, 8.0 Hz, 1H), 7.52-7.43 |
| | | (m, 2H), 7.07 (dd, J = 1.6, 7.2 Hz, 1H), 5.40-5.17 (m, 1H), |
| | | 4.38 (dd, J = 2.4, 5.6 Hz, 2H), 4.31 (dd, J = 5.2, 8.0 Hz, 1H), |
| | | 3.98-3.90 (m, 1H), 3.84-3.76 (m, 1H), 3.10-3.00 (m, 1H), |
| | | 2.35 (s, 3H), 2.21-2.11 (m, 1H), 2.00-1.80 (m, 4H), 1.58 (qd, |
| | | J = 6.8, 13.2 Hz, 1H) |
| I-34 | 425.4 | δ 10.02 (s, 1H), 9.24 (dd, J = 1.2, 7.6 Hz, 1H), 8.52 (s, 1H), |
| | | 8.01 (d, J = 1.6 Hz, 1H), 7.81-7.74 (m, 1H), 7.48 (d, J = 8.4 |
| | | Hz, 1H), 7.36-7.29 (m, 1H), 5.38-5.19 (m, 1H), 4.02 (s, 3H), |
| | | 3.09-3.01 (m, 1H), 2.35 (s, 3H), 2.00-1.96 (m, 1H), 1.61- |
| | | 1.54 (m, 1H) |
| I-35 | 476.3 | δ 10.11 (s, 1H), 9.80 (s, 1H), 8.57 (s, 1H), 8.05-7.99 (m, 1H), |
| | | 7.81-7.77 (m, 1H), 7.50 (d, J = 8.0 Hz, 1H), 7.42 (s, 1H), 5.43- |
| | | 5.17 (m, 1H), 4.03 (s, 3H), 3.12-3.00 (m, 1H), 2.36 (s, 3H), |
| | | 2.03-1.88 (m, 1H), 1.65-1.54 (m, 1H). |
| I-36 | 458.1 | δ 10.10-10.01 (m, 1H), 9.47-9.38 (m, 1H), 8.63-8.56 (m, |
| | | 1H), 8.16-8.10 (m, 1H), 7.88-7.70 (m, 2H), 7.51 (d, J = 8.0 |
| | | Hz, 1H), 7.20-7.08 (m, 1H), 4.97 (dd, J = 6.0, 8.8 Hz, 2H), |
| | | 4.85 (t, J = 6.4 Hz, 2H), 4.75-4.66 (m, 1H), 3.86 (q, J = 11.2 |
| | | Hz, 2H), 2.37 (s, 3H) |
| I-37 | 384.2 | δ 2.44 (s, 3H), 3.90 (s, 3H), 6.90 (dd, J = 7.6, 2.56 Hz, 1H), |
| | | 7.19 (d, J = 2.8 Hz, 1H), 7.83 (d, J = 8.38 Hz, 1H), 7.96 (dd, J = |
| | | 8.4, 2.06 Hz, 1H), 8.39 (d, J = 2.00 Hz, 1H), 8.51 (s, 1H), 9.26 |
| | | (d, J = 7.6 Hz, 1H), 10.11 (s, 1H) |
| I-38 | 446.2 | δ 10.43-10.36 (m, 1H), 9.61-9.51 (m, 1H), 8.73 (s, 1H), 8.35 |
| | | (d, J = 1.6 Hz, 1H), 8.06 (s, 1H), 7.99-7.93 (m, 1H), 7.84- |
| | | 7.79 (m, 1H), 7.37-7.05 (m, 2H), 5.03-4.92 (m, 2H), 4.90- |
| | | 4.81 (m, 2H), 4.78-4.67 (m, 1H) |
| I-39 | 414.1 | δ 9.73 (s, 1H), 8.85 (d, J = 7.2 Hz, 1H), 8.78 (s, 1H), 8.24 (d, J = |
| | | 8.8 Hz, 1H), 8.11 (d, J = 1.6 Hz, 1H), 7.84-7.75 (m, 1H), |
| | | 7.60-7.37 (m, 2H), 7.18-7.04 (m, 1H), 6.23-5.89 (m, 2H), |
| | | 4.33-4.16 (m, 1H), 3.27 (d, J = 3.6 Hz, 1H), 3.19-3.10 (m, |
| | | 1H), 2.36 (s, 3H) |
| I-40 | 444.2 | δ 9.90 (s, 1H), 9.28 (d, J = 7.6 Hz, 1H), 8.47 (s, 1H), 8.09 (s, |
| | | 1H), 7.82 (dd, J = 1.2, 8.0 Hz, 1H), 7.49 (d, J = 8.0 Hz, 1H), |
| | | 7.17 (d, J = 2.4 Hz, 1H), 6.88 (dd, J = 2.4, 7.6 Hz, 1H), 6.23- |
| | | 5.90 (m, 2H), 4.26 (dd, J = 5.4, 8.4 Hz, 1H), 3.90 (s, 3H), 3.32- |
| | | 3.27 (m, 1H), 3.21-3.10 (m, 1H), 2.37 (s, 3H) |
| I-41 | 476.3 | δ 10.15 (s, 1H), 9.74 (s, 1H), 8.55 (s, 1H), 7.99 (d, J = 1.6 Hz, |
| | | 1H), 7.78 (dd, J = 1.6, 8.0 Hz, 1H), 7.44 (s, 1H), 5.36-5.20 (m, |
| | | 1H), 4.01 (s, 3H), 3.11-3.00 (m, 1H), 2.35 (s, 3H), 2.00-1.88 |
| | | (m, 1H), 1.63-1.54 (m, 1H) |
| I-42 | 422.2 | δ 9.83 (s, 1H), 9.18 (s, 1H), 8.40 (s, 1H), 8.03 (d, J = 1.6 Hz, |
| | | 1H), 7.78-7.72 (m, 1H), 7.47 (d, J = 8.0 Hz, 1H), 7.14 (s, 1H), |
| | | 5.46-5.14 (m, 1H), 3.93 (s, 3H), 3.12-2.97 (m, 1H), 2.35 (s, |
| | | 3H), 2.19 (s, 3H), 2.03-1.85 (m, 1H), 1.63-1.52 (m, 1H) |
| I-43 | 440.2 | δ 10.14 (s, 1H), 9.53 (d, J = 7.2 Hz, 1H), 8.67 (s, 1H), 8.12 (d, J = |
| | | 1.6 Hz, 1H), 7.98 (s, 1H), 7.90-7.82 (m, 1H), 7.52 (d, J = 8.0 |
| | | Hz, 1H), 7.37-7.32 (m, 1H), 5.03-4.93 (m, 2H), 4.85 (t, J = |
| | | 6.4 Hz, 2H), 4.74-4.63 (m, 1H), 2.38 (s, 3H), 2.07 (t, J = 19.2 |
| | | Hz, 3H) |
| I-44 | 442.2 | δ 10.13 (s, 1H), 9.52 (d, J = 7.6 Hz, 1H), 8.66 (s, 1H), 8.06- |
| | | 7.90 (m, 2H), 7.81-7.73 (m, 1H), 7.49 (d, J = 8.0 Hz, 1H), |
| | | 7.39-7.29 (m, 1H), 5.56-5.10 (m, 1H), 3.12-2.98 (m, 1H), |
| | | 2.36 (s, 3H), 2.07 (t, J = 19.2 Hz, 3H), 2.00-1.87 (m, 1H), 1.66- |
| | | 1.51 (m, 1H) |
| I-45 | 460.1 | δ 10.06 (s, 1H), 9.43 (d, J = 7.2 Hz, 1H), 8.59 (s, 1H), 8.02 (d, J = |
| | | 1.6 Hz, 1H), 7.82 (s, 1H), 7.78 (dd, J = 1.6, 8.0 Hz, 1H), 7.48 |
| | | (d, J = 8.0 Hz, 1H), 7.16 (d, J = 7.6 Hz, 1H), 5.40-5.16 (m, |
| | | 1H), 3.86 (q, J = 11.2 Hz, 2H), 3.10-3.00 (m, 1H), 2.35 (s, |
| | | 3H), 2.01-1.88 (m, 1H), 1.63-1.53 (m, 1H) |
| I-46 | 508.3 | δ 9.97 (s, 1H), 9.37 (d, J = 7.2 Hz, 1H), 8.54 (s, 1H), 8.02 (d, J = |
| | | 1.2 Hz, 1H), 7.77 (dd, J = 1.6, 7.6 Hz, 1H), 7.69 (s, 1H), 7.48 |
| | | (d, J = 7.6 Hz, 1H), 7.13-7.08 (m, 1H), 5.40-5.16 (m, 1H), |
| | | 4.62 (s, 2H), 4.27 (s, 1H), 3.12-2.98 (m, 1H), 2.35 (s, 3H), |
| | | 2.01-1.87 (m, 1H), 1.58 (dd, J = 6.4, 13.2 Hz, 1H), 1.22 (s, |
| | | 6H), 1.18 (s, 6H) |
| I-47 | 426.0 | δ 10.10 (s, 1H), 9.26 (d, J = 7.6 Hz, 1H), 8.51 (s, 1H), 8.33 (d, J = |
| | | 2.0 Hz, 1H), 7.95-7.90 (m, 1H), 7.79 (d, J = 8.4 Hz, 1H), |
| | | 7.19 (d, J = 2.8 Hz, 1H), 6.91-6.88 (m, 1H), 5.00-4.95 (m, |
| | | 2H), 4.85 (t, J = 6.4 Hz, 2H), 4.76-4.69 (m, 1H), 3.91 (s, 3H) |
| I-48 | 408.1 | δ 10.06 (s, 1H), 9.47 (d, J = 7.2 Hz, 1H), 8.61 (s, 1H), 8.13 (d, J = |
| | | 1.6 Hz, 1H), 7.89-7.83 (m, 1H), 7.81 (s, 1H), 7.51 (d, J = 8.0 |
| | | Hz, 1H), 7.23-7.17 (m, 1H), 5.64 (s, 1H), 5.52 (s, 1H), 5.01- |
| | | 4.93 (m, 2H), 4.85 (t, J = 6.4 Hz, 2H), 4.75-4.67 (m, 1H), 2.38 |
| | | (s, 3H). |
| I-49 | 410.2 | δ 10.06 (s, 1H), 9.46 (d, J = 7.2 Hz, 1H), 8.60 (s, 1H), 8.02 (d, J = |
| | | 1.6 Hz, 1H), 7.84-7.73 (m, 2H), 7.48 (d, J = 8.0 Hz, 1H), |
| | | 7.25-7.16 (m, 1H), 5.63 (s, 1H), 5.51 (s, 1H), 5.41-5.12 (m, |
| | | 1H), 3.13-2.96 (m, 1H), 2.36 (s, 3H), 2.01-1.85 (m, 1H), 1.65- |
| | | 1.51 (m, 1H) |
| I-50 | 450.3 | δ 10.01 (s, 1H), 9.41 (d, J = 7.2 Hz, 1H), 8.57 (s, 1H), 8.13 (d, J = |
| | | 1.6 Hz, 1H), 7.85 (dd, J = 1.6, 8.0 Hz, 1H), 7.73 (s, 1H), 7.51 |
| | | (d, J = 8.0 Hz, 1H), 7.14 (dd, J = 1.6, 7.2 Hz, 1H), 5.18 (s, 1H), |
| | | 4.99-4.95 (m, 2H), 4.87-4.82 (m, 2H), 4.73-4.71 (m, 1H), |
| | | 4.64 (s, 2H), 3.62-3.57 (m, 2H), 3.57-3.52 (m, 2H), 2.38 (s, |
| | | 3H) |
| I-51 | 406.1 | δ 10.60 (s, 1H), 8.97-8.92 (m, 2H), 8.50 (s, 1H), 8.48-8.42 |
| | | (m, 1H), 7.75-7.70 (m, 1H), 7.59-7.54 (m, 1H), 7.50 (d, J = |
| | | 8.0 Hz, 1H), 2.67 (s, 3H), 2.52 (s, 3H) |
| I-52 | 426.1 | δ 10.17 (s, 1H), 9.57 (d, J = 7.2 Hz, 1H), 8.69 (s, 1H), 8.14 (d, J = |
| | | 1.6 Hz, 1H), 8.05 (s, 1H), 7.90-7.85 (m, 1H), 7.53 (d, J = 8.0 |
| | | Hz, 1H), 7.36-7.04 (m, 2H), 5.02-4.94 (m, 2H), 4.86 (t, J = |
| | | 6.4 Hz, 2H), 4.76-4.67 (m, 1H), 2.39 (s, 3H) |
| I-53 | 412.0 | δ 10.72 (s, 1H), 9.40 (d, J = 7.6 Hz, 1H), 8.87 (s, 1H), 7.91- |
| | | 7.79 (m, 2H), 7.45 (t, J = 8.0 Hz, 1H), 7.37 (d, J = 2.4 Hz, 1H), |
| | | 7.22 (dd, J = 2.4, 7.6 Hz, 1H), 5.43-5.17 (m, 1H), 4.00 (s, 3H), |
| | | 3.15-3.05 (m, 1H), 2.04-1.91 (m, 1H), 1.60 (qd, J = 6.8, 13.2 |
| | | Hz, 1H) |
| I-54 | 444.0 | δ 10.19 (s, 1H), 9.26 (d, J = 7.6 Hz, 1H), 8.51 (s, 1H), 7.97- |
| | | 7.79 (m, 2H), 7.44 (t, J = 8.0 Hz, 1H), 7.18 (d, J = 2.4 Hz, 1H), |
| | | 6.89 (dd, J = 2.4, 7.6 Hz, 1H), 3.90 (s, 3H), 3.27 (d, J = 7.2 Hz, |
| | | 2H), 2.29-2.15 (m, 1H), 1.85-1.71 (m, 1H), 1.58-1.40 (m, |
| | | 1H) |
| I-55 | 444.0 | δ 10.19 (d, J = 3.6, 1H), 9.26 (d, J = 7.6 Hz, 1H), 8.51 (s, 1H), |
| | | 7.94-7.78 (m, 2H), 7.44 (t, J = 8.0 Hz, 1H), 7.18 (d, J = 2.4 |
| | | Hz, 1H), 6.89 (dd, J = 2.4, 7.6 Hz, 1H), 3.90 (s, 3H), 3.27 (d, J = |
| | | 7.2 Hz, 2H), 2.29-2.15 (m, 1H), 1.84-1.71 (m, 1H), 1.55- |
| | | 1.40 (m, 1H) |
| I-56 | 472.1 | δ 10.21 (s, 1H), 9.38 (d, J = 7.2 Hz, 1H), 8.60 (s, 1H), 8.24 (d, J = |
| | | 2.0 Hz, 1H), 7.86 (dd, J = 2.0, 8.4 Hz, 1H), 7.79-7.72 (m, |
| | | 2H), 7.15 (dd, J = 1.6, 7.2 Hz, 1H), 5.40-5.19 (m, 1H), 4.73 (t, |
| | | J = 5.6 Hz, 1H), 4.64 (s, 2H), 3.61-3.57 (m, 2H), 3.56-3.53 |
| | | (m, 2H), 3.14-3.03 (m, 1H), 2.02-1.89 (m, 1H), 1.65-1.55 |
| | | (m, 1H) |
| I-57 | 456.3 | 8 10.75 (s, 1H), 9.52 (d, J = 7.2 Hz, 1H), 8.95 (s, 1H), 7.92 (s, |
| | | 1H), 7.90-7.83 (m, 2H), 7.48-7.41 (m, 2H), 5.42-5.18 (m, |
| | | 1H), 4.74 (s, 2H), 3.62-3.58 (m, 4H), 3.10 (dddd, J = 1.6, 6.8, |
| | | 11.2, 16.4 Hz, 1H), 2.06-1.89 (m, 1H), 1.60 (qd, J = 6.8, 13.2 |
| | | Hz, 1H) |
| I-58 | 470.0 | δ 10.02 (s, 1H), 9.40 (d, J = 7.2 Hz, 1H), 8.57 (s, 1H), 8.06 (s, |
| | | 1H), 7.81 (dd, J = 1.2, 8.0 Hz, 1H), 7.72 (s, 1H), 7.50 (d, J = |
| | | 8.0 Hz, 1H), 7.13 (d, J = 7.2 Hz, 1H), 4.72 (t, J = 4.8 Hz, 1H), |
| | | 4.63 (s, 2H), 3.70 (t, J = 8.2, 11.2 Hz, 1H), 3.58 (t, J = 4.4 Hz, |
| | | 2H), 3.56-3.52 (m, 2H), 2.49-2.42 (m, 2H), 2.36 (s, 3H) |
| I-59 | 466.3 | δ 10.04-9.96 (m, 1H), 9.42-9.34 (m, 1H), 8.58-8.53 (m, 1H), |
| | | 8.02 (d, J = 1.2 Hz, 1H), 7.77 (dd, J = 1.6, 8.0 Hz, 1H), 7.74- |
| | | 7.70 (m, 1H), 7.48 (d, J = 8.0 Hz, 1H), 7.13 (dd, J = 1.6, 7.2 |
| | | Hz, 1H), 5.40-5.15 (m, 1H), 4.76-4.69 (m, 1H), 4.69-4.60 |
| | | (m, 2H), 3.88-3.78 (m, 1H), 3.41-3.39 (m, 1H), 3.32-3.28 |
| | | (m, 1H), 3.10-2.99 (m, 1H), 2.35 (s, 3H), 2.01-1.87 (m, 1H), |
| | | 1.58 (qd, J = 6.8, 13.2 Hz, 1H), 1.16-1.04 (m, 3H) |
| I-60 | 428.1 | δ 10.02 (s, 1H), 9.04 (d, J = 7.2 Hz, 1H), 8.76 (s, 1H), 8.06- |
| | | 7.74 (m, 4H), 7.46 (d, J = 8.2 Hz, 1H), 7.27-7.22 (m, 1H), |
| | | 5.42-5.13 (m, 1H), 3.12-2.96 (m, 1H), 2.34 (s, 3H), 2.00- |
| | | 1.88 (m, 1H), 1.63-1.53 (m, 1H) |
| I-61 | 434.1 | δ 10.23 (s, 1H), 9.45 (d, J = 6.8 Hz, 1H), 8.64 (s, 1H), 8.31 (d, J = |
| | | 2.0 Hz, 1H), 7.95-7.88 (m, 1H), 7.84-7.75 (m, 2H), 7.59- |
| | | 7.51 (m, 1H), 7.24-7.17 (m, 1H), 6.24-5.88 (m, 2H), 4.32- |
| | | 4.19 (m, 1H), 3.30 (d, J = 4.4 Hz, 1H), 3.23-3.09 (m, 1H) |
| I-62 | 434.1 | δ 10.23 (s, 1H), 9.45 (d, J = 6.8 Hz, 1H), 8.64 (s, 1H), 8.31 (d, J = |
| | | 2.0 Hz, 1H), 7.95-7.89 (m, 1H), 7.83-7.76 (m, 2H), 7.58- |
| | | 7.52 (m, 1H), 7.23-7.17 (m, 1H), 6.22-5.90 (m, 2H), 4.32- |
| | | 4.20 (m, 1H), 3.30 (d, J = 4.0 Hz, 1H), 3.21-3.14 (m, 1H) |
| I-63 | 480.2 | δ 9.92 (s, 1H), 9.27 (s, 1H), 8.94 (s, 1H), 8.41 (d, J = 9.2 Hz, |
| | | 1H), 8.10 (d, J = 1.6 Hz, 1H), 7.85-7.80 (m, 1H), 7.79-7.65 |
| | | (m, 1H), 7.53 (d, J = 8.0 Hz, 1H), 7.42-7.04 (m, 1H), 5.48- |
| | | 5.23 (m, 1H), 3.23 (d, J = 5.2 Hz, 1H), 3.17-3.03 (m, 1H), |
| | | 2.07-1.92 (m, 1H), 1.71-1.58 (m, 1H) |
| I-64 | 480.2 | 8 10.45 (s, 1H) 9.55 (d, J = 6.8 Hz, 1H) 8.92 (s, 1H) 8.02 (s, 1H) |
| | | 7.93 (s, 1H) 7.81 (m, 1H) 7.51 (d, J = 8.0 Hz, 1H) 7.44 (d, J = 6.8 |
| | | Hz, 1H) 5.18-5.39 (m, 1H) 4.77 (s, 2H) 3.32 (s, 2H) 3.02- |
| | | 3.10 (m, 1H) 2.35-2.42 (m, 3H)3H) 1.90-2.01 (m, 1H) 1.55- |
| | | 1.64 (m, 2H) 1.14-1.24 (m, 6 H) |
| I-65 | 444.2 | δ 10.57 (s, 1H) 9.25 (d, J = 2.4 Hz, 1H) 9.03 (s, 1H) 8.07 (d, |
| | | J = 1.6 Hz, 1H) 7.97 (d, J = 9.6 Hz, 1H) 7.86 (m, 1H) 7.72 (m, |
| | | 1H) 7.52 (d, J = 8.0 Hz, 1H) 5.92-6.20 (m, 1H) 4.21-4.27 (m, |
| | | 1H) 3.90 (s, 3H) 3.30 (m, 1H) 3.11-3.20 (m, 1H) 2.39 (s, |
| | | 3H)3H) |
| I-66 | 444.2 | δ 10.50 (s, 1H) 9.24 (d, J = 2.4 Hz, 1H) 8.97 (s, 1H) 8.07 (d, |
| | | J = 1.6 Hz, 1H) 7.95 (d, J = 9.6 Hz, 1H) 7.85 (m, 1H) 7.69 (m, |
| | | 1H) 7.52 (d, J = 8.0 Hz, 1H) 5.88-6.24 (m, 1H) 4.22-4.27 (m, |
| | | 1H) 3.89 (s, 3H) 3.30 (m, 1H) 3.12-3.19 (m, 1H) 2.39 (s, |
| | | 3H)3H) |
| I-67 | 432.2 | δ 10.03 (s, 1H), 9.46 (d, J = 7.0 Hz, 1H), 8.60 (s, 1H), 8.10 (d, |
| | | J = 1.2 Hz, 1H), 7.85-7.75 (m, 2H), 7.55-7.48 (m, 2H), 7.18 (t, |
| | | J = 6.8 Hz, 1H), 6.86 (s, 1H), 4.64 (s, 1H), 3.50-3.39 (m, 1H), |
| | | 3.31-3.22 (m, 1H), 2.37 (s, 3H) |
| I-68 | 432.2 | δ 10.03 (s, 1H), 9.46 (d, J = 7.2 Hz, 1H), 8.60 (s, 1H), 8.10 (d, |
| | | J = 1.2 Hz, 1H), 7.85-7.75 (m, 2H), 7.55-7.48 (m, 2H), 7.18 (t, |
| | | J = 6.8 Hz, 1H), 6.86 (s, 1H), 4.64 (s, 1H), 3.50-3.39 (m, 1H), |
| | | 3.31-3.22 (m, 1H), 2.37 (s, 3H) |
| I-69 | 464.2 | δ 10.18 (s, 1H), 9.57 (d, J = 7.2 Hz, 1H), 8.70 (s, 1H), 8.14- |
| | | 8.02 (m, 2H), 7.86-7.81 (m, 1H), 7.52 (d, J = 8.0 Hz, 1H), |
| | | 7.35 (s, 1H), 7.35-7.32 (m, 1H), 7.19 (s, 1H), 7.05 (s, 1H), |
| | | 6.23-5.89 (m, 2H), 4.35-4.19 (m, 1H), 3.28 (d, J = 3.6 Hz, |
| | | 1H), 3.20-3.12 (m, 1H), 2.38 (s, 3H) |
| I-70 | 464.2 | δ 10.19-10.15 (m, 1H), 9.57 (d, J = 7.6 Hz, 1H), 8.69 (s, 1H), |
| | | 8.13-8.04 (m, 2H), 7.87-7.82 (m, 1H), 7.52 (d, J = 8.0 Hz, |
| | | 1H), 7.39-7.01 (m, 2H), 6.01 (s, 2H), 4.35-4.14 (m, 1H), 3.32- |
| | | 3.27 (m, 1H), 3.21-3.09 (m, 1H), 2.38 (s, 3H) |
| I-447 | 475.3 | δ = 10.13 (s, 1H), 9.70 (s, 1H), 8.64 (s, 1H), 8.01 (s, 1H), 7.85- |
| | | 7.73 (m, 2H), 7.68 (d, J = 9.2 Hz, 1H), 7.49 (d, J = 8.0 Hz, 1H), |
| | | 5.40-5.17 (m, 1H), 3.52 (d, J = 6.8 Hz, 4H), 3.13-2.99 (m, |
| | | 1H), 2.36 (s, 3H), 1.97-1.81 (m, 5H), 1.65-1.55 (m, 1H). |
| I-448 | 416.9 | δ = 9.98 (s, 1H), 9.38 (d, J = 7.2 Hz, 1H), 8.55 (s, 1H), 8.09 (d, |
| | | J = 1.2 Hz, 1H), 7.97 (s, 1H), 7.81-7.77 (m, 1H), 7.66 (s, 1H), |
| | | 7.48 (d, J = 8.0 Hz, 1H), 7.11-7.07 (m, 1H), 3.75 (t, J = 9.2 |
| | | Hz, 1H), 3.40-3.33 (m, 2H), 2.66 (s, 3H), 2.59-2.51 (m, 1H), |
| | | 2.36 (s, 3H), 2.27-2.16 (m, 1H). |
| I-449 | 449.3 | δ = 10.22-10.02 (m, 1H), 9.56 (s, 1H), 8.65 (s, 1H), 8.02 (s, |
| | | 1H), 7.89-7.75 (m, 2H), 7.64-7.55 (m, 1H), 7.53-7.44 (m, |
| | | 1H), 5.40-5.18 (m, 1H), 3.34-3.33 (m, 1H), 3.12-3.04 (m, |
| | | 1H), 3.02 (s, 6H), 2.36 (s, 3H), 2.03-1.88 (m, 1H), 1.65-1.55 |
| | | (m, 1H) |
| I-456 | 436.0 | δ = 9.97 (s, 1H), 9.34 (d, J = 7.2 Hz, 1H), 8.54 (s, 1H), 8.02 (d, |
| | | J = 1.2 Hz, 1H), 7.82-7.69 (m, 2H), 7.48 (d, J = 8.0 Hz, 1H), |
| | | 7.29 (dd, J = 1.6, 7.2 Hz, 1H), 5.42-5.17 (m, 2H), 3.12-2.98 |
| | | (m, 1H), 2.35 (s, 3H), 2.03-1.87 (m, 1H), 1.58 (dd, J = 6.4, |
| | | 13.2 Hz, 1H), 1.49 (s, 6H) |
| I-458 | 454.4 | δ = 10.02 (s, 1H), 9.19 (dd, J = 1.2, 7.6 Hz, 1H), 8.51 (s, 1H), |
| | | 8.00 (d, J = 1.6 Hz, 1H), 7.77 (dd, J = 1.6, 8.0 Hz, 1H), 7.48 (d, |
| | | J = 8.0 Hz, 1H), 7.28 (t, J = 7.6 Hz, 1H), 5.41-5.17 (m, 1H), |
| | | 4.83 (td, J = 6.0, 12.0 Hz, 1H), 3.15-2.96 (m, 1H), 2.34 (s, |
| | | 3H), 2.02-1.86 (m, 1H), 1.58 (qd, J = 6.8, 13.2 Hz, 1H), 1.34 |
| | | (d, J = 6.0 Hz, 6H) |
| I-459 | 422.2 | δ = 9.97 (s, 1H), 9.34 (d, J = 7.2 Hz, 1H), 8.53 (s, 1H), 8.03 (d, |
| | | J = 1.6 Hz, 1H), 7.78 (m, 1H), 7.65-7.57 (m, 1H), 7.48 (d, J = |
| | | 8.0 Hz, 1H), 7.09 (m, 1H), 5.41-5.18 (m, 1H), 4.75 (t, J = 5.2 |
| | | Hz, 1H), 3.75-3.67 (m, 2H), 3.06 (m, 1H), 2.85 (t, J = 6.4 Hz, |
| | | 2H), 2.36 (s, 3H), 2.03-1.86 (m, 1H), 1.59 (m, 1H). |
| I-460 | 450.4 | 400 MHz, CDCl3 δ = 9.44 (d, J = 7.2 Hz, 1H), 8.48 (s, 1H), |
| | | 8.26 (s, 1H), 7.85-7.74 (m, 2H), 7.61 (s, 1H), 7.37 (d, J = 8.0 |
| | | Hz, 1H), 7.09-6.99 (m, 1H), 5.22-4.91 (m, 1H), 2.89 (s, 2H), |
| | | 2.80-2.65 (m, 1H), 2.43 (s, 3H), 1.91-1.83 (m, 2H), 1.68- |
| | | 1.58 (m, 1H), 1.31 (s, 6H). |
| I-461 | 452.2 | 400 MHz, CDCl3 δ = 8.58-8.53 (m, 2H), 8.36 (d, J = 9.0 Hz, |
| | | 1H), 8.25 (s, 1H), 7.83-7.76 (m, 1H), 7.47 (s, 1H), 7.43-7.39 |
| | | (m, 1H), 7.35 (d, J = 8.0 Hz, 1H), 5.19-4.96 (m, 1H), 4.65 (s, |
| | | 2H), 3.88-3.81 (m, 2H), 3.73-3.64 (m, 2H), 2.78-2.67 (m, |
| | | 1H), 2.42 (s, 3H), 1.90-1.77 (m, 1H), 1.65-1.58 (m, 1H). |
| I-462 | 520.2 | δ = 10.13-9.78 (m, 1H), 9.40 (d, J = 7.2 Hz, 1H), 8.57 (s, 1H), |
| | | 8.02 (d, J = 1.6 Hz, 1H), 7.85-7.67 (m, 2H), 7.48 (d, J = 8.0 |
| | | Hz, 1H), 7.26-7.03 (m, 1H), 6.64-6.38 (m, 1H), 5.44-5.12 |
| | | (m, 1H), 4.69 (s, 2H), 4.30-4.20 (m, 1H), 3.77-3.66 (m, 1H), |
| | | 3.66-3.54 (m, 1H), 3.11-2.99 (m, 1H), 2.35 (s, 3H), 2.02- |
| | | 1.83 (m, 1H), 1.67-1.46 (m, 1H). |
| I-463 | 520.2 | δ = 10.13-9.78 (m, 1H), 9.40 (d, J = 7.2 Hz, 1H), 8.57 (s, 1H), |
| | | 8.02 (d, J = 1.6 Hz, 1H), 7.85-7.67 (m, 2H), 7.48 (d, J = 8.0 |
| | | Hz, 1H), 7.26-7.03 (m, 1H), 6.64-6.38 (m, 1H), 5.44-5.12 |
| | | (m, 1H), 4.69 (s, 2H), 4.30-4.20 (m, 1H), 3.77-3.66 (m, 1H), |
| | | 3.66-3.54 (m, 1H), 3.11-2.99 (m, 1H), 2.35 (s, 3H), 2.02- |
| | | 1.83 (m, 1H), 1.67-1.46 (m, 1H). |
| I-464 | 450.2 | δ = 9.96 (s, 1H), 9.31 (d, J = 7.6 Hz, 1H), 8.53 (s, 1H), 8.02 (d, |
| | | J = 1.6 Hz, 1H), 7.80-7.74 (m, 1H), 7.60 (d, J = 1.2 Hz, 1H), |
| | | 7.48 (d, J = 8.0 Hz, 1H), 7.31-7.23 (m, 1H), 5.42-5.15 (m, |
| | | 1H), 4.78 (t, J = 5.6 Hz, 1H), 3.51 (d, J = 5.6 Hz, 2H), 3.19- |
| | | 2.95 (m, 1H), 2.35 (s, 3H), 2.07-1.84 (m, 1H), 1.68-1.50 (m, |
| | | 1H), 1.28 (s, 6H) |
| I-465 | 494.4 | δ = 10.02 (s, 1H), 9.41 (d, J = 7.2 Hz, 1H), 8.57 (s, 1H), 8.03 (d, |
| | | J = 1.6 Hz, 1H), 7.81-7.75 (m, 1H), 7.69 (s, 1H), 7.49 (d, J = |
| | | 8.0 Hz, 1H), 7.17-7.08 (m, 1H), 5.43-5.16 (m, 1H), 4.65 (s, |
| | | 2H), 3.38 (s, 2H), 3.15 (s, 3H), 3.11-3.01 (m, 1H), 2.36 (s, |
| | | 3H), 2.02-1.88 (m, 1H), 1.66-1.54 (m, 1H), 1.15 (s, 6H) |
| I-472 | 470.2 | δ = 9.31 (d, J = 7.2 Hz, 1H), 8.47 (s, 1H), 8.07 (d, J = 1.6 Hz, |
| | | 1H), 7.86 (dd, J = 1.6, 7.6 Hz, 1H), 7.46 (d, J = 8.0 Hz, 1H), |
| | | 7.29 (t, J = 6.4 Hz, 1H), 5.25-4.99 (m, 1H), 4.80 (d, J = 1.6 |
| | | Hz, 2H), 3.77-3.71 (m, 2H), 3.69-3.63 (m, 2H), 2.91-2.80 |
| | | (m, 1H), 2.40 (s, 3H), 1.96-1.83 (m, 1H), 1.67-1.55 (m, 1H) |
| I-473 | 498.3 | δ = 10.15 (s, 1H), 9.27 (d, J = 7.2 Hz, 1H), 8.59 (s, 1H), 8.02 |
| | | (d, J = 1.6 Hz, 1H), 7.79 (m, 1H), 7.50 (d, J = 8.0 Hz, 1H), 7.25- |
| | | 7.20 (m, 1H), 5.42-5.17 (m, 1H), 4.73 (d, J = 1.2 Hz, 2H), |
| | | 4.43 (s, 1H), 3.26 (s, 2H), 2.36 (s, 3H), 2.01-1.88 (m, 1H), |
| | | 1.59 (m, 1H), 1.10 (s, 6H). |
| I-476 | 414.0 | δ = 10.55 (s, 1H), 9.62 (d, J = 6.8 Hz, 1H), 9.03 (s, 1H), 8.08 |
| | | (d, J = 1.6 Hz, 1H), 8.03 (d, J = 9.2 Hz, 1H), 7.96-7.90 (m, |
| | | 1H), 7.86 (dd, J = 1.6, 7.9 Hz, 1H), 7.54-7.48 (m, 2H), 5.99- |
| | | 5.99 (m, 1H), 6.35-5.76 (m, 1H), 4.29-4.20 (m, 1H), 3.35- |
| | | 3.24 (m, 1H), 3.20-3.09 (m, 1H), 2.39 (s, 3H). |
| I-479 | 448.0 | δ = 9.90 (s, 1H), 8.89 (s, 1H), 8.43 (s, 1H), 7.99 (d, J = 1.6 Hz, |
| | | 1H), 7.77 (dd, J = 2.0, 8.0 Hz, 1H), 7.61 (d, J = 0.8 Hz, 1H), |
| | | 7.47 (d, J = 8.0 Hz, 1H), 5.42-5.16 (m, 1H), 3.17 (d, J = 1.2 |
| | | Hz, 2H), 3.06 (m, 1H), 2.33 (s, 3H), 2.01-1.88 (m, 1H), 1.59 |
| | | (m, 1H), 1.47 (s, 6H) |
| I-484 | 490.0 | δ = 9.96 (s, 1H), 9.31 (d, J = 7.6 Hz, 1H), 8.49 (s, 1H), 8.00 (d, |
| | | J = 0.8 Hz, 1H), 7.77 (dd, J = 1.4, 8.0 Hz, 1H), 7.53-7.44 (m, |
| | | 2H), 6.97 (dd, J = 2.4, 7.6 Hz, 1H), 5.58-5.52 (m, 1H), 5.41- |
| | | 5.17 (m, 1H), 3.12-2.99 (m, 1H), 2.34 (s, 3H), 2.01-1.87 (m, |
| | | 1H), 1.63-1.54 (m 1H), 1.49 (d, J = 6.4 Hz, 3H). |
| I-485 | 490.1 | δ = 9.96 (s, 1H), 9.31 (d, J = 7.6 Hz, 1H), 8.49 (s, 1H), 8.00 (s, |
| | | 1H), 7.77 (d, J = 8.4 Hz, 1H), 7.52-7.45 (m, 2H), 6.97 (dd, J = |
| | | 2.4, 7.7 Hz, 1H), 5.61-5.49 (m, 1H), 5.40-5.17 (m, 1H), 3.12- |
| | | 3.00 (m, 1H), 2.34 (s, 3H), 2.01-1.87 (m, 1H), 1.61-1.56 (m, |
| | | 1H), 1.49 (d, J = 6.4 Hz, 3H). |
| I-487 | 396.1 | (400 MHz, CD₃OD) δ = 8.58 (d, J = 6.8 Hz, 1H), 8.50 (s, 1H), |
| | | 8.19 (d, J = 0.8 Hz, 1H), 7.83 (dd, J = 1.6, 8.0 Hz, 1H), 7.43 (d, |
| | | J = 8.0 Hz, 1H), 7.28 (dd, J = 8.0, 11.2 Hz, 1H), 7.11-7.03 (m, |
| | | 1H), 5.26-5.00 (m, 1H), 2.92-2.75 (m, 1H), 2.42 (s, 3H), 1.98- |
| | | 1.81 (m, 1H), 1.68-1.54 (m, 1H) |
| I-491 | 422.2 | δ = 9.98 (s, 1H), 9.30 (d, J = 7.2 Hz, 1H), 8.57 (s, 1H), 8.02 (d, |
| | | J = 6.8 Hz, 2H), 7.77 (dd, J = 1.2, 7.6 Hz, 1H), 7.57 (d, J = 6.4 |
| | | Hz, 1H), 7.48 (d, J = 8.0 Hz, 1H), 5.44-5.16 (m, 1H), 3.11- |
| | | 3.00 (m, 1H), 2.36 (s, 3H), 2.00-1.86 (m, 1H), 1.66-1.51 (m, |
| | | 1H). |
| I-492 | 448.1 | δ = 9.82 (s, 1H), 9.27 (s, 1H), 8.38 (s, 1H), 8.02 (d, J = 1.6 Hz, |
| | | 1H), 7.87-7.68 (m, 1H), 7.47 (d, J = 8.0 Hz, 1H), 6.91 (s, 1H), |
| | | 5.47-5.14 (m, 1H), 3.15 (s, 2H), 3.10-3.00 (m, 1H), 2.35 (s, |
| | | 3H), 2.03-1.87 (m, 1H), 1.63-1.54 (m, 1H), 1.48 (s, 6H) |
| I-495 | 470.2 | δ = 10.09 (s, 1H), 9.43 (d, J = 5.6 Hz, 1H), 8.61 (s, 1H), 8.01 |
| | | (d, J = 1.2 Hz, 1H), 7.88 (d, J = 6.8 Hz, 1H), 7.81-7.76 (m, |
| | | 1H), 7.49 (d, J = 8.0 Hz, 1H), 5.39-5.18 (m, 1H), 4.81-4.67 |
| | | (m, 3H), 3.59 (s, 4H), 3.10-3.01 (m, 1H), 2.35 (s, 3H), 2.01- |
| | | 1.88 (m, 1H), 1.64-1.53 (m, 1H). |
| I-497 | 396.2 | δ = 9.87 (s, 1H), 8.90 (s, 1H), 8.10 (m, 1H), 8.04 (d, J = 1.6 Hz, |
| | | 1H), 7.77 (m, 1H), 7.61 (m, 1H), 7.48 (d, J = 7.6 Hz, 1H), 7.11 |
| | | (t, J = 6.4 Hz, 1H), 5.17-5.42 (m, 1H), 3.00-3.13 (m, 1H), 2.36 |
| | | (s, 3H), 1.85-2.02 (m, 1H), 1.53-1.64 (m, 1H) |
| I-502 | 494.3 | (400 MHz, CD₃OD) δ = 8.58 (s, 2H), 8.19 (d, J = 9.2 Hz, 1H), |
| | | 8.06 (s, 1H), 7.88-7.79 (m, 1H), 7.56-7.36 (m, 2H), 5.26-4.99 |
| | | (m, 1H), 3.78 (t, J = 6.0 Hz, 2H), 3.29 (s, 2H), 2.98 (t, J = 6.0 |
| | | Hz, 2H), 2.90-2.76 (m, 1H), 2.40 (s, 3H), 1.98-1.77 (m, 1H), |
| | | 1.67-1.53 (m, 1H), 1.16 (s, 6H) |
| I-507 | 436.2 | (400 MHz, CD₃OD) δ = 9.46 (d, J = 7.2 Hz, 1H), 8.45 (s, 1H), |
| | | 8.09 (d, J = 1.6 Hz, 1H), 7.88 (dd, J = 2.0, 8.0 Hz, 1H), 7.73 (s, |
| | | 1H), 7.46 (d, J = 8.0 Hz, 1H), 7.16 (dd, J = 1.2, 7.2 Hz, 1H), |
| | | 4.71 (s, 2H), 3.41 (s, 2H), 2.65 (s, 3H), 2.41 (s, 3H), 1.26 (s, |
| | | 6H) |
| I-508 | 516.3 | (400 MHz, CD₃OD) δ = 9.46 (d, J = 7.2 Hz, 1H), 8.46 (s, 1H), |
| | | 8.13 (d, J = 1.6 Hz, 1H), 7.91 (dd, J = 2.0, 8.0 Hz, 1H), 7.73 (s, |
| | | 1H), 7.47 (d, J = 8.0 Hz, 1H), 7.17 (dd, J = 1.2, 7.2 Hz, 1H), |
| | | 6.07-5.75 (m, 1H), 4.71 (s, 2H), 4.42-4.24 (m, 1H), 3.41 (s, |
| | | 2H), 3.29-3.12 (m, 2H), 2.41 (s, 3H), 1.26 (s, 6H) |
| I-509 | 516.3 | 400 MHz, CD₃OD) δ = 9.46 (d, J = 7.2 Hz, 1H), 8.46 (s, 1H), |
| | | 8.13 (d, J = 1.6 Hz, 1H), 7.91 (dd, J = 1.6, 7.6 Hz, 1H), 7.73 (s, |
| | | 1H), 7.47 (d, J = 8.0 Hz, 1H), 7.17 (dd, J = 1.2, 7.2 Hz, 1H), |
| | | 6.07-5.73 (m, 1H), 4.71 (s, 2H), 4.41-4.27 (m, 1H), 3.41 (s, |
| | | 2H), 3.30-3.15 (m, 2H), 2.41 (s, 3H), 1.26 (s, 6H) |
| I-510 | 488.3 | δ = 10.06-9.96 (m, 1H), 9.40 (d, J = 7.6 Hz, 1H), 8.57 (s, 1H), |
| | | 8.10 (d, J = 1.6 Hz, 1H), 7.88-7.78 (m, 1H), 7.72 (s, 1H), 7.50 |
| | | (d, J = 8.0 Hz, 1H), 7.20-7.06 (m, 1H), 6.21-5.87 (m, 2H), |
| | | 4.72 (t, J = 5.6 Hz, 1H), 4.64 (s, 2H), 4.36-4.19 (m, 1H), 3.62- |
| | | 3.57 (m, 2H), 3.57-3.52 (m, 2H), 3.28 (d, J = 4.0 Hz, 1H), |
| | | 3.18-3.11 (m, 1H), 2.37 (s, 3H) |
| I-511 | 488.2 | δ = 10.00 (s, 1H), 9.45-9.33 (m, 1H), 8.57 (s, 1H), 8.10 (d, J = |
| | | 1.6 Hz, 1H), 7.82 (dd, J = 1.6, 8.0 Hz, 1H), 7.72 (s, 1H), 7.50 |
| | | (d, J = 8.0 Hz, 1H), 7.13 (dd, J = 1.6, 7.2 Hz, 1H), 6.23-5.87 |
| | | (m, 2H), 4.72 (t, J = 5.6 Hz, 1H), 4.63 (s, 2H), 4.34-4.17 (m, |
| | | 1H), 3.64-3.51 (m, 4H), 3.31-3.25 (m, 1H), 3.20-3.11 (m, |
| | | 1H), 2.37 (s, 3H) |
| I-512 | 446.3 | δ = 10.03 (s, 1H), 9.46 (d, J = 7.2 Hz, 1H), 8.59 (s, 1H), 8.09 (d, |
| | | J = 1.6 Hz, 1H), 7.87-7.76 (m, 2H), 7.57-7.44 (m, 2H), 7.22- |
| | | 7.15 (m, 1H), 6.54 (s, 1H), 3.37 (d, J = 10.4 Hz, 2H), 2.37 (s, |
| | | 3H), 1.48 (s, 3H) |
| I-513 | 460.2 | δ = 10.04 (s, 1H), 9.45 (d, J = 7.2 Hz, 1H), 8.58 (s, 1H), 8.06 |
| | | (d, J = 1.6 Hz, 1H), 7.85-7.75 (m, 2H), 7.56-7.45 (m, 2H), |
| | | 7.18-7.16 (m, 1H), 6.10 (s, 1H), 3.22-3.01 (m, 2H), 2.35 (s, |
| | | 3H), 2.24-2.14 (m, 1H), 2.13-2.02 (m, 1H), 1.30 (s, 3H) |
| I-514 | 396.2 | δ = 9.67 (s, 1H), 7.95 (d, J = 1.6 Hz, 1H), 7.78 (s, 1H), 7.74 (m, |
| | | 1H), 7.44 (d, J = 8.0 Hz, 1H), 5.15-5.41 (m, 1H), 4.39-4.57 (m, |
| | | 1H), 3.93-4.06 (m, 1H), 3.01-3.10 (m, 1H), 2.89-2.98 (m, 1H), |
| | | 2.39 (m, 1H), 2.30 (s, 3H), 1.87-2.02 (m, 3H), 1.51-1.65 (m, |
| | | 2H), 1.07 (d, J = 6.4 Hz, 3H) |
| I-520 | 410.2 | δ = 9.68 (s, 1H), 7.97 (d, J = 1.2 Hz, 1H), 7.82 (s, 1H), 7.73 |
| | | (dd, J = 1.6, 8.0 Hz, 1H), 7.43 (d, J = 8.0 Hz, 1H), 5.41-5.17 |
| | | (m, 1H), 3.99 (s, 2H), 3.13-2.99 (m, 1H), 2.83 (t, J = 6.8 Hz, |
| | | 2H), 2.30 (s, 3H), 2.01-1.85 (m, 1H), 1.67 (t, J = 6.8 Hz, 2H), |
| | | 1.61-1.55 (m, 1H), 1.01 (s, 6H) |
| I-522 | 470.1 | (400 MHz, CD₃OD) δ = 7.99 (d, J = 1.6 Hz, 1H), 7.82 (dd, J = |
| | | 1.8, 8.0 Hz, 1H), 7.75 (s, 1H), 7.42 (d, J = 8.0 Hz, 1H), 5.27- |
| | | 5.00 (m, 1H), 4.67-4.58 (m, 1H), 4.19-4.04 (m, 1H), 3.66- |
| | | 3.60 (m, 2H), 3.59-3.54 (m, 2H), 3.53 (d, J = 6.0 Hz, 2H), |
| | | 3.38 (s, 3H), 3.07 (dd, J = 4.4, 17.2 Hz, 1H), 2.90-2.77 (m, 1H), |
| | | 2.61 (dd, J = 10.4, 17.2 Hz, 1H), 2.35 (s, 3H), 2.32-2.22 (m, |
| | | 1H), 2.22-2.15 (m, 1H), 1.98-1.82 (m, 1H), 1.82-1.70 (m, |
| | | 1H), 1.67-1.54 (m, 1H) |
| I-523 | 442.1 | δ = 10.02 (s, 1H), 9.46 (d, J = 6.8 Hz, 1H), 8.59 (s, 1H), 8.07 |
| | | (d, J = 1.6 Hz, 1H), 7.85-7.76 (m, 2H), 7.56-7.45 (m, 2H), |
| | | 7.18 (m, 1H), 5.97-5.65 (m, 1H), 5.41 (s, 1H), 3.10 (m, 2H), |
| | | 2.36 (s, 3H), 2.11-1.88 (m, 2H), 1.16 (s, 3H) |
| I-524 | 404.1 | δ = 10.04 (s, 1H), 10.18-9.97 (m, 1H), 9.46 (dd, J = 1.2, 6.8 |
| | | Hz, 1H), 8.59 (s, 1H), 8.06 (s, 1H), 7.86-7.73 (m, 2H), 7.56- |
| | | 7.45 (m, 2H), 7.25-7.10 (m, 1H), 5.33-4.86 (m, 1H), 4.51- |
| | | 4.26 (m, 1H), 3.29-3.22 (m, 1H), 2.36 (s, 3H), 2.24-1.61 (m, |
| | | 6H) |
| I-527 | 456.2 | δ = 9.67 (s, 1H), 7.94 (d, J = 1.6 Hz, 1H), 7.79 (s, 1H), 7.73 |
| | | (dd, J = 1.6, 8.0 Hz, 1H), 7.43 (d, J = 8.0 Hz, 1H), 5.43-5.15 |
| | | (m, 1H), 4.61 (s, 1H), 4.55-4.48 (m, 1H), 4.06-3.92 (m, 1H), |
| | | 3.56-3.48 (m, 2H), 3.47-3.40 (m, 4H), 3.10-2.99 (m, 1H), 2.94 |
| | | (dd, J = 4.8, 16.8 Hz, 1H), 2.46 (d, J = 10.8 Hz, 1H), 2.29 (s, |
| | | 3H), 2.17 (td, J = 2.7, 5.2 Hz, 1H), 2.09 (d, J = 13.4 Hz, 1H), |
| | | 2.01-1.87 (m, 1H), 1.69-1.52 (m, 2H) |
| I-528 | 408.3 | δ = 10.07-10.00 (m, 1H), 9.49-9.43 (m, 1H), 8.60 (s, 1H), |
| | | 8.09 (d, J = 1.6 Hz, 1H), 7.87-7.75 (m, 2H), 7.59-7.47 (m, |
| | | 2H), 7.25-7.10 (m, 1H), 5.29 (d, J = 5.6 Hz, 1H), 4.24-4.06 |
| | | (m, 1H), 3.39 (d, J = 5.6 Hz, 2H), 3.30 (s, 3H), 3.17 (dd, J = |
| | | 4.4, 15.2 Hz, 1H), 3.02 (dd, J = 8.4, 15.2 Hz, 1H), 2.37 (s, 3H) |
| I-534 | 348.1 | (400 MHz, CD₃OD) δ = 9.37 (d, J = 7.2 Hz, 1H), 8.41 (s, 1H), |
| | | 8.08 (d, J = 1.2 Hz, 1H), 7.94-7.84 (m, 1H), 7.51 (s, 1H), 7.46 |
| | | (d, J = 8.0 Hz, 1H), 7.04 (d, J = 7.2 Hz, 1H), 2.65 (s, 3H), 2.50 |
| | | (s, 3H), 2.40 (s, 3H) |
| I-538 | 440.1 | δ = 10.03 (s, 1H), 9.45 (d, J = 7.2 Hz, 1H), 8.59 (s, 1H), 8.08 |
| | | (d, J = 1.2 Hz, 1H), 7.85-7.76 (m, 2H), 7.55-7.48 (m, 2H), |
| | | 7.18 (t, J = 6.8 Hz, 1H), 6.25 (s, 1H), 6.20-5.90 (m, 1H), 3.57- |
| | | 3.50 (m, 1H), 2.84-2.77 (m, 2H), 2.53 (d, J = 2.0 Hz, 1H), |
| | | 2.47 (s, 1H), 2.37 (s, 3H). |
| I-539 | 416.3 | δ = 9.96 (s, 1H), 9.32 (d, J = 7.2 Hz, 1H), 8.51 (s, 1H), 8.05 (d, |
| | | J = 1.2 Hz, 1H), 7.83-7.76 (m, 1H), 7.56 (s, 1H), 7.47 (d, J = |
| | | 8.0 Hz, 1H), 7.06-6.99 (m, 1H), 3.16-3.05 (m, 1H), 2.42 (s, |
| | | 3H), 2.35 (s, 3H), 2.15-2.02 (m, 2H), 1.80-1.70 (m, 2H), 1.69- |
| | | 1.55 (m, 3H), 1.50-1.21 (m, 3H). |
| I-543 | 463.3 | δ = 10.02 (s, 1H), 9.45 (d, J = 6.8 Hz, 1H), 8.59 (s, 1H), 8.06 (s, |
| | | 1H), 7.83-7.76 (m, 2H), 7.55-7.47 (m, 2H), 7.18 (t, J = 6.8 |
| | | Hz, 1H), 5.14 (d, J = 4.8 Hz, 1H), 4.19 (t, J = 6.0 Hz, 1H), 3.44 |
| | | (d, J = 3.2 Hz, 4H), 3.21-3.13 (m, 1H), 3.08-3.00 (m, 1H), |
| | | 2.46-2.28 (m, 9H). |
| I-544 | 425.1 | δ = 9.98 (s, 1H), 9.34 (d, J = 7.2 Hz, 1H), 8.69 (s, 1H), 8.54 (s, |
| | | 1H), 8.24 (d, J = 8.0 Hz, 1H), 8.19 (s, 1H), 7.95-7.88 (m, 2H), |
| | | 7.57 (s, 1H), 7.53 (d, J = 8.0 Hz, 1H), 7.03 (d, J = 7.2 Hz, 1H), |
| | | 2.43 (d, J = 3.6 Hz, 6H), 2.38 (s, 3H). |
| I-545 | 428.1 | δ = 9.94 (s, 1H), 9.32 (d, J = 7.2 Hz, 1H), 8.52 (s, 1H), 8.09 (s, |
| | | 1H), 7.83-7.79 (m, 1H), 7.56 (s, 1H), 7.49 (d, J = 8.0 Hz, 1H), |
| | | 7.03 (d, J = 6.4 Hz, 1H), 6.21-5.89 (m, 2H), 4.32-4.19 (m, |
| | | 1H), 3.31-3.26 (m, 1H), 3.19-3.10 (m, 1H), 2.42 (s, 3H), 2.36 |
| | | (s, 3H). |
| I-546 | 412.1 | δ = 10.00 (s, 1H), 9.88 (s, 1H), 9.60 (d, J = 5.2 Hz, 1H), 9.33 |
| | | (d, J = 7.2 Hz, 1H), 8.54 (s, 1H), 8.40-8.36 (m, 1H), 8.19 (s, |
| | | 1H), 7.95-7.90 (m, 1H), 7.59-7.52 (m, 2H), 7.06-7.01 (m, |
| | | 1H), 2.43 (s, 3H), 2.39 (s, 3H). |
| I-550 | 412.1 | δ = 9.99 (s, 1H), 9.52 (dd, J = 1.6, 5.2 Hz, 1H), 9.34 (d, J = 7.2 |
| | | Hz, 1H), 8.57-8.51 (m, 2H), 8.23 (d, J = 1.6 Hz, 1H), 8.04 (dd, |
| | | J = 5.2, 8.8 Hz, 1H), 7.94 (dd, J = 1.6, 8.0 Hz, 1H), 7.59-7.52 |
| | | (m, 2H), 7.03 (dd, J = 1.6, 7.2 Hz, 1H), 2.43 (s, 3H), 2.40 (s, |
| | | 3H) |
| I-551 | 429.1 | δ = 9.99 (s, 1H), 9.33 (d, J = 7.2 Hz, 1H), 8.53 (s, 1H), 8.13 (s, |
| | | 1H), 7.88 (d, J = 8.0 Hz, 1H), 7.57 (s, 1H), 7.52 (d, J = 8.0 Hz, |
| | | 1H), 7.03 (d, J = 7.2 Hz, 1H), 2.74 (s, 3H), 2.49 (s, 3H), 2.43 (s, |
| | | 3H), 2.37 (s, 3H). |
| I-557 | 407.4 | δ = 10.05 (s, 1H), 9.46 (d, J = 7.0 Hz, 1H), 8.60 (s, 1H), 8.32 |
| | | (d, J = 1.6 Hz, 1H), 8.08 (d, J = 1.6 Hz, 1H), 7.87-7.74 (m, |
| | | 2H), 7.60-7.45 (m, 2H), 7.24-7.10 (m, 1H), 4.23 (s, 2H), |
| | | 3.26-3.18 (m, 1H), 3.13-3.01 (m, 1H), 2.94-2.73 (m, 2H), |
| | | 2.44 (s, 3H), 2.37 (s, 3H) |
| I-558 | 435.3 | δ = 10.02 (s, 1H), 9.46 (d, J = 7.0 Hz, 1H), 8.60 (s, 1H), 8.08 |
| | | (d, J = 1.6 Hz, 1H), 7.99 (t, J = 5.7 Hz, 1H), 7.83-7.76 (m, |
| | | 2H), 7.68 (s, 1H), 7.55-7.44 (m, 2H), 7.22-7.14 (m, 1H), 7.03 |
| | | (s, 1H), 5.32 (d, J = 4.2 Hz, 1H), 4.08 (s, 1H), 3.22-3.09 (m, |
| | | 3H), 3.02-2.92 (m, 1H), 2.37 (s, 3H), 1.83 (s, 3H) |
| I-563 | 406.2 | δ = 9.73 (s, 1H), 8.90-8.83 (m, 1H), 8.78 (s, 1H), 8.30-8.20 |
| | | (m, 1H), 8.10 (d, J = 1.6 Hz, 1H), 7.80-7.76 (m, 1H), 7.56- |
| | | 7.51 (m, 1H), 7.47 (d, J = 8.0 Hz, 1H), 7.16-7.10 (m, 1H), |
| | | 4.43 (s, 1H), 3.07-2.98 (m, 2H), 2.36 (s, 3H), 1.95-1.85 (m, |
| | | 2H), 1.15 (s, 6H) |
| I-564 | 337.2 | δ = 9.34 (d, J = 1.6 Hz, 1H), 8.94 (d, J = 1.6 Hz, 1H), 8.53 (s, |
| | | 1H), 3.87 (s, 3H), 2.22 (br t, J = 5.2 Hz, 1H), 1.04-0.93 (m, |
| | | 4H) |
| I-565 | 336.3 | δ = 10.01 (s, 1H), 9.46 (d, J = 7.2 Hz, 1H), 8.59 (s, 1H), 8.08 |
| | | (d, J = 1.6 Hz, 1H), 7.84-7.74 (m, 2H), 7.57-7.45 (m, 2H), |
| | | 7.23-7.13 (m, 1H), 2.67-2.61 (m, 1H), 2.36 (s, 3H). |
| I-567 | 508.2 | δ = 9.99 (s, 1H), 9.41 (d, J = 7.2 Hz, 1H), 8.56 (s, 1H), 8.07 (s, |
| | | 1H), 7.80 (d, J = 7.6 Hz, 1H), 7.72 (s, 1H), 7.48 (d, J = 8.0 Hz, |
| | | 1H), 7.13 (d, J = 7.6 Hz, 1H), 4.65 (s, 2H), 4.45 (s, 1H), 4.41 (s, |
| | | 1H), 3.27 (s, 2H), 3.07-2.99 (m, 2H), 2.36 (s, 3H), 1.94-1.85 |
| | | (m, 2H), 1.14 (d, J = 2.8 Hz, 12H) |
| I-569 | 384.1 | δ = 9.84 (s, 1H), 9.20 (s, 1H), 8.88 (s, 1H), 8.35 (d, J = 9.2 Hz, |
| | | 1H), 8.09 (d, J = 1.6 Hz, 1H), 7.78 (dd, J = 1.6, 8.0 Hz, 1H), |
| | | 7.68 (d, J = 9.2 Hz, 1H), 7.47 (d, J = 8.0 Hz, 1H), 7.16 (t, J = |
| | | 54.8 Hz, 1H), 2.66 (s, 3H), 2.36 (s, 3H) |
| I-572 | 432.1 | δ = 9.96 (s, 1H), 9.32 (d, J = 6.8 Hz, 1H), 8.52 (s, 1H), 8.11 (d, |
| | | J = 1.6 Hz, 1H), 7.97 (d, J = 5.2 Hz, 1H), 7.90-7.78 (m, 1H), |
| | | 7.57 (s, 1H), 7.52 (d, J = 8.0 Hz, 1H), 7.05-7.00 (m, 1H), 6.03- |
| | | 5.90 (m, 1H), 2.43 (s, 3H), 2.38 (s, 3H) |
| I-573 | 499.3 | δ = 10.04 (s, 1H), 9.43 (d, J = 7.2 Hz, 1H), 8.86 (d, J = 4.0 Hz, |
| | | 1H), 8.59 (s, 1H), 8.35 (d, J = 8.0 Hz, 1H), 8.21 (d, J = 1.6 Hz, |
| | | 1H), 8.16-8.09 (m, 1H), 7.93 (dd, J = 1.6, 8.0 Hz, 1H), 7.78- |
| | | 7.71 (m, 2H), 7.55 (d, J = 8.0 Hz, 1H), 7.14 (dd, J = 1.6, 7.2 |
| | | Hz, 1H), 4.66 (s, 2H), 4.45 (s, 1H), 3.28 (s, 2H), 2.40 (s, 3H), |
| | | 1.14 (s, 6H) |
| I-577 | 471.2 | δ = 10.06 (s, 1H), 9.43 (d, J = 7.2 Hz, 1H), 8.89-8.83 (m, 1H), |
| | | 8.61 (s, 1H), 8.35 (d, J = 8.0 Hz, 1H), 8.21 (d, J = 1.6 Hz, 1H), |
| | | 8.16-8.10 (m, 1H), 7.94-7.92 (m, 1H), 7.77-7.71 (m, 2H), 7.55 |
| | | (d, J = 8.0 Hz, 1H), 7.16 (dd, J = 1.6, 7.2 Hz, 1H), 4.65 (s, 2H), |
| | | 3.61-3.53 (m, 4H), 2.40 (s, 3H) |
| I-578 | 462.1 | δ = 9.98 (s, 1H), 9.35 (d, J = 7.1 Hz, 1H), 8.56 (s, 1H), 8.10 (d, |
| | | J = 1.6 Hz, 1H), 7.86-7.79 (m, 1H), 7.63-7.53 (m, 3H), 7.50 |
| | | (d, J = 8.1 Hz, 1H), 7.10-7.02 (m, 1H), 3.45 (s, 2H), 2.44 (s, |
| | | 3H), 2.34 (s, 3H) |
| I-579 | 448.1 | δ = 9.96 (s, 1H), 9.32 (d, J = 7.2 Hz, 1H), 8.93 (s, 2H), 8.52 (s, |
| | | 1H), 8.12 (d, J = 1.6 Hz, 1H), 7.84 (dd, J = 1.6, 8.0 Hz, 1H), |
| | | 7.57 (s, 1H), 7.52 (d, J = 8.0 Hz, 1H), 7.03 (dd, J = 1.6, 7.2 Hz, |
| | | 1H), 2.43 (s, 3H), 2.38 (s, 3H) |
| I-581 | 515.1 | δ = 10.06 (s, 1H), 9.42 (d, J = 7.2 Hz, 1H), 8.61 (s, 1H), 8.09 (s, |
| | | 1H), 7.82 (d, J = 8.0 Hz, 1H), 7.75 (s, 1H), 7.50 (d, J = 8.0 Hz, |
| | | 1H), 7.14 (d, J = 6.8 Hz, 1H), 6.24-5.90 (m, 2H), 4.65 (s, 2H), |
| | | 4.29-4.20 (m, 1H), 3.74 (d, J = 4.4 Hz, 2H), 3.28 (d, J = 3.6 |
| | | Hz, 1H), 3.17 (d, J = 9.2 Hz, 1H), 2.99 (s, 2H), 2.54 (s, 6H), |
| | | 2.37 (s, 3H) |
| I-584 | 444.1 | δ = 9.94 (s, 1H), 9.33 (d, J = 7.2 Hz, 1H), 8.52 (s, 1H), 8.08 (d, J = |
| | | 1.6 z, 1H), 7.81 (dd, J = 1.6, 8.0 Hz, 1H), 7.56 (s, 1H), 7.49 |
| | | (d, J = 8.0 Hz, 1H), 7.03 (dd, J = 1.2, 7.2 Hz, 1H), 6.87 (s, 2H), |
| | | 5.84 (s, 1H), 3.31 (s, 2H), 2.43 (s, 3H), 2.36 (s, 3H) |
| I-588 | 516.2 | δ = 9.97 (s, 1H), 9.37 (d, J = 7.2 Hz, 1H), 8.55 (s, 1H), 8.10 (d, |
| | | J = 1.2 Hz, 1H), 7.82 (dd, J = 1.6, 8.0 Hz, 1H), 7.70 (s, 1H), |
| | | 7.50 (d, J = 8.0 Hz, 1H), 7.12 (dd, J = 1.2, 7.2 Hz, 1H), 6.22- |
| | | 5.82 (m, 2H), 4.74 (t, J = 5.6 Hz, 1H), 4.59 (s, 2H), 4.34-4.15 |
| | | (m, 1H), 3.38 (d, J = 6.0 Hz, 2H), 3.29-3.11 (m, 2H), 2.37 (s, |
| | | 3H), 1.19 (s, 6H) |
| I-589 | 408.1 | δ = 9.02 (d, J = 7.2 Hz, 1H), 8.54 (s, 1H), 7.75 (s, 1H), 7.16 |
| | | (dd, J = 1.6, 7.2 Hz, 1H), 7.06 (s, 2H), 6.82 (d, J = 7.8 Hz, 1H), |
| | | 6.62 (dd, J = 1.6, 7.6 Hz, 1H), 5.43 (s, 1H), 5.31 (s, 1H), 5.01 |
| | | (s, 2H), 2.47 (s, 3H), 2.04 (s, 3H) |
| I-590 | 487.1 | δ = 10.06 (s, 1H), 9.42 (d, J = 7.2 Hz, 1H), 8.59 (s, 1H), 8.10 |
| | | (d, J = 1.6 Hz, 1H), 7.83 (dd, J = 1.6, 8.0 Hz, 1H), 7.78 (s, 1H), |
| | | 7.50 (d, J = 8.0 Hz, 1H), 7.16 (dd, J = 1.2, 7.2 Hz, 1H), 6.31- |
| | | 5.87 (m, 2H), 4.65 (s, 2H), 4.33-4.15 (m, 2H), 3.62-3.58 (m, |
| | | 3H), 3.30 (dd, J = 4.0, 15.6 Hz, 1H), 3.19-3.15 (m, 1H), 2.95- |
| | | 2.89 (m, 2H), 2.37 (s, 3H) |
| I-591 | 420.2 | δ = 10.04 (s, 1H), 9.48 (d, J = 7.2 Hz, 1H), 8.62 (s, 1H), 8.12 |
| | | (d, J = 1.1 Hz, 1H), 7.89-7.77 (m, 2H), 7.60-7.48 (m, 2H), |
| | | 7.19 (t, J = 6.8 Hz, 1H), 5.44 (d, J = 5.6 Hz, 1H), 4.66-4.56 |
| | | (m, 2H), 4.55-4.49 (m, 1H), 4.40 (t, J = 6.4 Hz, 1H), 4.33- |
| | | 4.24 (m, 1H), 3.17-3.05 (m, 2H), 2.98 (d, J = 8.4 Hz, 1H), |
| | | 2.39 (s, 3H) |
| I-593 | 501.2 | δ = 10.02 (s, 1H), 9.43 (d, J = 7.2 Hz, 1H), 8.58 (s, 1H), 8.09 (s, |
| | | 1H), 7.86-7.79 (m, 2H), 7.50 (d, J = 8.0 Hz, 1H), 7.16 (d, J = |
| | | 7.2 Hz, 1H), 6.24-5.89 (m, 2H), 4.68 (s, 2H), 4.32-4.15 (m, |
| | | 1H), 3.74 (t, J = 4.8 Hz, 2H), 3.28 (d, J = 4.0 Hz, 2H), 3.19- |
| | | 3.12 (m, 2H), 2.61 (s, 3H), 2.37 (s, 3H) |
| I-598 | 515.2 | δ = 10.06 (s, 1H), 9.41 (d, J = 7.2 Hz, 1H), 8.58 (s, 1H), 8.36 (s, |
| | | 1H), 8.09 (d, J = 1.2 Hz, 1H), 7.82 (dd, J = 1.6, 8.0 Hz, 1H), |
| | | 7.78 (s, 1H), 7.50 (d, J = 8.0 Hz, 1H), 7.15 (dd, J = 1.2, 7.2 Hz, |
| | | 1H), 6.22-5.91 (m, 1H), 4.69-4.62 (m, 2H), 4.30-4.22 (m, |
| | | 2H), 3.59-3.51 (m, 2H), 3.29 (m, 1H), 3.20-3.08 (m, 2H), |
| | | 2.45 (s, 3H), 2.37 (s, 3H), 1.13 (d, J = 6.4 Hz, 3H) |
| I-599 | 515.3 | δ = 10.08 (s, 1H), 9.42 (d, J = 6.4 Hz, 1H), 8.60 (s, 1H), 8.38 (s, |
| | | 1H), 8.09 (s, 1H), 7.91-7.73 (m, 2H), 7.49 (d, J = 8.0 Hz, 1H), |
| | | 7.14 (d, J = 6.8 Hz, 1H), 6.21-5.92 (m, 1H), 4.65 (s, 2H), 4.32- |
| | | 4.21 (m, 1H), 3.57 (s, 2H), 3.34-3.25 (m, 1H), 3.22-3.09 (m, |
| | | 2H), 2.46 (s, 3H), 2.37 (s, 3H), 1.15 (s, 3H) |
| I-600 | 615.3 | δ = 10.00 (s, 1H), 9.40 (d, J = 7.2 Hz, 1H), 8.56 (s, 1H), 8.09 (s, |
| | | 1H), 7.84-7.80 (m, 1H), 7.66 (s, 1H), 7.50 (d, J = 8.0 Hz, 1H), |
| | | 7.09 (d, J = 7.2 Hz, 1H), 6.21-5.90 (m, 2H), 4.69-4.52 (m, |
| | | 2H), 4.46-4.18 (m, 2H), 3.56-3.40 (m, 2H), 3.30-3.10 (m, |
| | | 2H), 2.67 (s, 3H), 2.37 (s, 3H), 1.38 (s, 9H), 1.05 (s, 3H) |
| I-604 | 414.0 | δ = 10.11 (s, 1H), 9.44 (dd, J = 0.6, 7.6 Hz, 1H), 8.60 (s, 1H), |
| | | 8.09 (d, J = 1.6 Hz, 1H), 7.99 (dd, J = 0.8, 2.0 Hz, 1H), 7.83 |
| | | (dd, J = 1.6, 8.0 Hz, 1H), 7.50 (d, J = 8.0 Hz, 1H), 7.26 (dd, J = |
| | | 2.4, 7.6 Hz, 1H), 6.41-6.08 (m, 1H), 5.30-4.96 (m, 1H), 4.91 |
| | | (t, J = 6.0 Hz, 1H), 3.78 (d, J = 6.0 Hz, 2H), 2.36 (s, 3H) |
| I-605 | 430.1 | δ = 10.15 (s, 1H), 9.64 (d, J = 7.2 Hz, 1H), 8.60 (s, 1H), 8.06- |
| | | 7.91 (m, 2H), 7.79 (dd, J = 1.6, 8.0 Hz, 1H), 7.49 (d, J = 8.0 |
| | | Hz, 1H), 5.41-5.19 (m, 1H), 3.13-3.00 (m, 1H), 2.35 (s, 3H), |
| | | 2.00-1.90 (m, 1H), 1.58 (dd, J = 6.4, 13.2 Hz, 1H) |
| I-606 | 396.1 | δ = 10.05 (s, 1H), 9.58-9.43 (m, 1H), 8.58 (s, 1H), 8.07 (s, |
| | | 1H), 7.82 (dd, J = 1.6, 8.0 Hz, 1H), 7.68 (dd, J = 2.4, 9.6 Hz, |
| | | 1H), 7.49 (d, J = 8.0 Hz, 1H), 7.27-7.19 (m, 1H), 5.03 (d, J = |
| | | 5.2 Hz, 1H), 4.26-4.08 (m, 1H), 3.14-2.98 (m, 2H), 2.36 (s, |
| | | 3H), 1.22 (d, J = 6.4 Hz, 3H) |
| I-607 | 408.1 | δ = 10.02 (s, 1H), 9.35 (d, J = 7.2 Hz, 1H), 8.57 (s, 1H), 8.09 |
| | | (d, J = 1.4 Hz, 1H), 7.82 (dd, J = 1.6, 8.0 Hz, 1H), 7.60 (s, 1H), |
| | | 7.50 (d, J = 8.0 Hz, 1H), 7.08 (dd, J = 1.6, 7.2 Hz, 1H), 6.41 (s, |
| | | 1H), 5.10 (t, J = 5.6 Hz, 1H), 3.80-3.72 (m, 2H), 3.30 (s, 3H), |
| | | 2.44 (s, 3H), 2.37 (s, 3H) |
| I-608 | 408.2 | δ = 10.22 (s, 1H), 9.41 (d, J = 7.2 Hz, 1H), 8.74 (s, 1H), 8.09 |
| | | (d, J = 1.2 Hz, 1H), 7.83 (dd, J = 1.6, 8.0 Hz, 1H), 7.70 (s, 1H), |
| | | 7.51 (d, J = 8.0 Hz, 1H), 7.22 (d, J = 7.2 Hz, 1H), 6.65-6.15 |
| | | (m, 1H), 5.10 (t, J = 5.6 Hz, 1H), 3.75 (d, J = 5.6 Hz, 2H), 3.30 |
| | | (s, 3H), 2.49-2.48 (m, 3H), 2.37 (s, 3H) |
| I-609 | 424.3 | δ = 9.49-9.41 (m, 1H), 8.61 (s, 1H), 8.08 (d, J = 1.6 Hz, 1H), |
| | | 7.98 (d, J = 2.1 Hz, 1H), 7.91-7.81 (m, 1H), 7.50 (d, J = 8.0 |
| | | Hz, 1H), 7.32-7.23 (m, 1H), 5.65 (s, 1H), 3.20 (s, 2H), 2.37 (s, |
| | | 3H), 0.78-0.65 (m, 4H) |
| I-610 | 463.1 | δ = 10.13 (s, 1H), 9.65 (d, J = 7.2 Hz, 1H), 8.60 (s, 1H), 8.03- |
| | | 7.95 (m, 2H), 7.74 (dd, J = 1.6, 7.6 Hz, 1H), 7.46 (d, J = 8.0 |
| | | Hz, 1H), 4.78-4.72 (m, 4H), 2.35 (s, 3H) |
| I-611 | 459.1 | δ = 9.95 (s, 1H), 9.41 (d, J = 6.8 Hz, 1H), 8.48 (s, 1H), 7.95 (d, |
| | | J = 1.6 Hz, 1H), 7.71 (dd, J = 1.6, 8.0 Hz, 1H), 7.42 (dd, J = |
| | | 8.4, 15.6 Hz, 2H), 4.74 (t, J = 12.4 Hz, 4H), 4.00 (s, 3H), 2.33 |
| | | (s, 3H) |
| I-612 | 408.3 | δ = 10.06 (s, 1H), 9.57-9.43 (m, 1H), 8.57 (s, 1H), 8.07 (d, J = |
| | | 1.2 Hz, 1H), 7.86-7.79 (m, 1H), 7.72-7.63 (m, 1H), 7.49 (d, |
| | | J = 8.0 Hz, 1H), 7.27-7.18 (m, 1H), 5.65 (s, 1H), 3.19 (s, 2H), |
| | | 2.36 (s, 3H), 0.70 (d, J = 3.6 Hz, 4H) |
| I-614 | 414.1 | δ = 10.12 (s, 1H), 9.44 (d, J = 7.6 Hz, 1H), 8.62 (s, 1H), 8.09 |
| | | (d, J = 1.2 Hz, 1H), 7.99 (d, J = 2.0 Hz, 1H), 7.83 (dd, J = 1.6, |
| | | 8.0 Hz, 1H), 7.50 (d, J = 8.0 Hz, 1H), 7.27 (dd, J = 2.4, 7.6 Hz, |
| | | 1H), 4.91 (t, J = 6.0 Hz, 1H), 3.78 (d, J = 6.0 Hz, 2H), 2.37 (s, |
| | | 3H) |
| I-617 | 529.3 | δ = 9.57 (d, J = 7.2 Hz, 1H), 9.06-8.99 (m, 1H), 8.14-8.00 |
| | | (m, 2H), 7.84 (dd, J = 1.6, 8.0 Hz, 1H), 7.60-7.47 (m, 2H), |
| | | 6.23-5.86 (m, 1H), 4.82 (s, 2H), 4.29-4.16 (m, 1H), 3.60 (s, |
| | | 3H), 3.33-3.23 (m, 1H), 3.20-3.09 (m, 1H), 2.49 (s, 2H), 2.37 |
| | | (s, 3H), 1.28 (s, 6H) |
| I-618 | 506.4 | δ = 10.01 (s, 1H), 9.41 (d, J = 7.2 Hz, 1H), 8.57 (s, 1H), 8.08 (s, |
| | | 1H), 7.81 (d, J = 8.0 Hz, 1H), 7.72 (s, 1H), 7.49 (d, J = 8.0 Hz, |
| | | 1H), 7.13 (d, J = 7.2 Hz, 1H), 4.65 (s, 2H), 4.46 (s, 1H), 4.35- |
| | | 4.30 (m, 1H), 3.77 (q, J = 7.2 Hz, 1H), 3.68-3.58 (m, 1H), |
| | | 3.27 (s, 2H), 3.24-3.11 (m, 2H), 2.36 (s, 3H), 2.09 (dd, J = 5.2, |
| | | 13.2 Hz, 1H), 1.90-1.76 (m, 2H), 1.69-1.60 (m, 1H), 1.14 (s, |
| | | 6H) |
| I-619 | 463.2 | δ = 10.15 (s, 1H), 9.60 (d, J = 5.2 Hz, 1H), 8.63 (s, 1H), 8.24 |
| | | (d, J = 7.2 Hz, 1H), 7.95 (d, J = 1.2 Hz, 1H), 7.76-7.71 (m, |
| | | 1H), 7.46 (d, J = 8.0 Hz, 1H), 4.74 (t, J = 12.4 Hz, 4H), 2.34 (s, |
| | | 3H) |
| I-620 | 473.2 | δ = 10.03 (s, 1H), 9.38 (s, 1H), 8.56 (s, 1H), 8.03-7.93 (m, |
| | | 2H), 7.75-7.70 (m, 1H), 7.45 (d, J = 8.0 Hz, 1H), 4.75 (t, J = |
| | | 12.4 Hz, 4H), 2.80-2.73 (m, 2H), 2.34 (s, 3H), 1.24 (t, J = 7.2 |
| | | Hz, 3H) |
| I-621 | 504.3 | δ = 10.02-9.93 (m, 1H), 9.38 (d, J = 7.2 Hz, 1H), 8.56 (s, 1H), |
| | | 8.09-8.06 (m, 1H), 7.80 (dd, J = 1.6, 8.0 Hz, 1H), 7.65 (s, 1H), |
| | | 7.49 (d, J = 8.0 Hz, 1H), 7.10 (dd, J = 1.6, 7.2 Hz, 1H), 4.85- |
| | | 4.76 (m, 1H), 4.72 (s, 2H), 4.35-4.24 (m, 1H), 3.81-3.73 (m, |
| | | 1H), 3.68-3.58 (m, 3H), 3.29-3.10 (m, 2H), 2.36 (s, 3H), 2.15- |
| | | 2.04 (m, 1H), 1.93-1.79 (m, 2H), 1.72-1.60 (m, 1H), 0.86- |
| | | 0.78 (m, 2H), 0.64-0.58 (m, 2H) |
| I-623 | 423.1 | δ = 10.11 (s, 1H), 9.43 (d, J = 7.2 Hz, 1H), 8.61 (s, 1H), 8.22 (s, |
| | | 1H), 8.07 (s, 1H), 7.99 (d, J = 2.0 Hz, 1H), 7.83 (dd, J = 1.6, |
| | | 8.0 Hz, 1H), 7.50 (d, J = 8.0 Hz, 1H), 7.27 (dd, J = 2.4, 7.6 Hz, |
| | | 1H), 3.96-3.85 (m, 2H), 2.70-2.62 (m, 2H), 2.52 (s, 2H), 2.36 |
| | | (s, 3H) |
| I-625 | 445.1 | δ = 9.81 (s, 1H), 9.22 (d, J = 0.8 Hz, 1H), 8.80 (s, 1H), 8.23 (d, |
| | | J = 9.6 Hz, 1H), 7.98 (d, J = 0.8 Hz, 1H), 7.72-7.68 (m, 1H), |
| | | 7.62-7.57 (m, 1H), 7.43 (d, J = 8.0 Hz, 1H), 4.74 (t, J = 12.4 |
| | | Hz, 4H), 2.33 (s, 3H) |
| I-626 | 429.1 | δ = 9.81 (s, 1H), 9.22 (d, J = 0.8 Hz, 1H), 8.80 (s, 1H), 8.23 (d, |
| | | J = 9.6 Hz, 1H), 7.98 (d, J = 0.8 Hz, 1H), 7.72-7.68 (m, 1H), |
| | | 7.62-7.57 (m, 1H), 7.43 (d, J = 8.0 Hz, 1H), 4.74 (t, J = 12.4 |
| | | Hz, 4H), 2.33 (s, 3H) |
| I-632 | 459.1 | δ = 9.85 (s, 1H), 9.26-9.19 (m, 1H), 8.81 (s, 1H), 8.23 (d, J = |
| | | 9.6 Hz, 1H), 8.08 (d, J = 1.6 Hz, 1H), 7.80 (dd, J = 1.6, 8.0 Hz, |
| | | 1H), 7.60 (dd, J = 1.6, 9.2 Hz, 1H), 7.48 (d, J = 8.0 Hz, 1H), |
| | | 4.89-4.78 (m, 1H), 3.90-3.75 (m, 1H), 3.31-3.22 (m, 2H), |
| | | 2.92-2.59 (m, 2H), 2.35 (s, 3H) |
| I-634 | 460.1 | δ = 10.22 (s, 1H), 9.55 (d, J = 6.8 Hz, 1H), 8.70 (s, 1H), 8.07- |
| | | 7.98 (m, 2H), 7.82-7.77 (m, 1H), 7.49 (d, J = 8.0 Hz, 1H), |
| | | 5.39-5.18 (m, 1H), 3.12-3.00 (m, 1H), 2.35 (s, 3H), 2.11 (t, J = |
| | | 19.2 Hz, 3H), 2.01-1.89 (m, 1H), 1.63-1.54 (m, 1H) |
| I-636 | 443.1 | δ = 9.83 (s, 1H), 9.24-9.17 (m, 1H), 8.80 (s, 1H), 8.26 (dd, J = |
| | | 5.6, 9.8 Hz, 1H), 8.08 (s, 1H), 7.80 (dd, J = 1.6, 8.0 Hz, 1H), |
| | | 7.72-7.58 (m, 1H), 7.48 (d, J = 8.0 Hz, 1H), 4.83 (q, J = 7.2 |
| | | Hz, 1H), 3.83 (q, J = 7.2 Hz, 1H), 3.31-3.18 (m, 2H), 2.87- |
| | | 2.61 (m, 2H), 2.35 (s, 3H) |
| I-637 | 490.3 | δ = 10.00 (s, 1H), 9.37 (d, J = 7.2 Hz, 1H), 8.55 (s, 1H), 8.03 |
| | | (d, J = 1.6 Hz, 1H), 7.79 (dd, J = 1.6, 8.0 Hz, 1H), 7.65 (s, 1H), |
| | | 7.48 (d, J = 8.0 Hz, 1H), 7.10 (dd, J = 1.6, 7.2 Hz, 1H), 4.80- |
| | | 4.68 (m, 4H), 4.50-4.40 (m, 2H), 3.61 (d, J = 4.8 Hz, 2H), |
| | | 3.55-3.38 (m, 4H), 2.35 (s, 3H), 0.86-0.78 (m, 2H), 0.67- |
| | | 0.57 (m, 2H) |
| I-639 | 479.2 | δ = 10.15 (s, 1H), 9.52 (d, J = 7.2 Hz, 1H), 8.68 (s, 1H), 8.11 |
| | | (d, J = 1.6 Hz, 1H), 8.00 (s, 1H), 7.85 (dd, J = 1.6, 8.0 Hz, 1H), |
| | | 7.53 (d, J = 8.0 Hz, 1H), 7.35 (dd, J = 1.6, 7.2 Hz, 1H), 5.10 (t, |
| | | J = 7.4 Hz, 1H), 3.20-3.09 (m, 2H), 2.38 (s, 3H), 2.32 (dd, J = |
| | | 5.6, 7.6 Hz, 2H), 2.08 (t, J = 19.2 Hz, 3H), 0.79-0.57 (m, 4H) |
| I-750 | 424.2 | 1H NMR (400 MHz, DMSO-d6) δ = 10.11 (s, 1H), 9.43 (d, J = |
| | | 7.2 Hz, 1H), 8.60 (s, 1H), 8.05 (d, J = 1.6 Hz, 1H), 7.98 (d, J = |
| | | 1.6 Hz, 1H), 7.82 (dd, J = 1.6, 8.0 Hz, 1H), 7.49 (d, J = 8.0 Hz, |
| | | 1H), 7.26 (dd, J = 2.4, 7.2 Hz, 1H), 5.38 (d, J = 6.4 Hz, 1H), |
| | | 4.51-4.40 (m, 1H), 3.82-3.65 (m, 1H), 2.65-2.55 (m, 2H), |
| | | 2.44-2.37 (m, 2H), 2.36 (s, 3H) |
| I-749 | 424.2 | 1H NMR (400 MHz, DMSO-d6) δ = 10.11 (s, 1H), 9.44 (d, J = |
| | | 7.2 Hz, 1H), 8.61 (s, 1H), 8.07 (s, 1H), 7.99 (d, J = 2.0 Hz, 1H), |
| | | 7.82 (dd, J = 1.6, 8.0 Hz, 1H), 7.50 (d, J = 8.0 Hz, 1H), 7.27 |
| | | (dd, J = 2.0, 7.2 Hz, 1H), 5.42 (d, J = 7.2 Hz, 1H), 4.24-4.09 |
| | | (m, 1H), 3.33-3.28 (m, 1H), 2.80-2.64 (m, 2H), 2.37 (s, 3H), |
| | | 2.32-2.17 (m, 2H) |
| I-748 | 420.2 | 1H NMR (400 MHz, DMSO-d6) δ = 9.89 (s, 1H), 9.27 (d, J = |
| | | 7.6 Hz, 1H), 8.45 (s, 1H), 8.05 (d, J = 1.6 Hz, 1H), 7.70-7.85 |
| | | (m, 1H), 7.47 (d, J = 7.9 Hz, 1H), 7.17 (d, J = 2.4 Hz, 1H), 6.85 |
| | | (dd, J = 2.4, 7.6 Hz, 1H), 5.25-5.17 (m, 1H), 3.98-3.90 (m, |
| | | 1H), 3.90-3.83 (m, 2H), 3.70-3.80 (m, 1H), 2.66 (s, 3H), 2.36- |
| | | 2.26 (m, 4H), 2.10-1.95 (m, 1H) |
| I-747 | 523.2 | 1H NMR (400 MHz, DMSO-d6) δ = 10.09 (s, 1H), 9.46-9.39 |
| | | (m, 1H), 8.60 (s, 1H), 8.06 (d, J = 1.2 Hz, 1H), 8.01-7.95 (m, |
| | | 1H), 7.81 (dd, J = 1.6, 8.0 Hz, 1H), 7.49 (d, J = 8.0 Hz, 1H), |
| | | 7.32 (d, J = 8.0 Hz, 1H), 7.26 (dd, J = 2.0, 7.2 Hz, 1H), 4.15- |
| | | 3.97 (m, 1H), 3.57-3.47 (m, 1H), 2.72-2.62 (m, 2H), 2.39- |
| | | 2.27 (m, 5H), 1.37 (s, 9H) |
| I-746 | 423.1 | 1H NMR (400 MHz, DMSO-d6) δ = 10.85 (s, 1H), 9.58 (d, J = |
| | | 7.6 Hz, 1H), 9.24 (s, 1H), 8.48 (s, 2H), 8.21 (d, J = 2.0 Hz, 1H), |
| | | 8.04 (d, J = 1.2 Hz, 1H), 7.83 (dd, J = 1.6, 8.0 Hz, 1H), 7.56 |
| | | (dd, J = 2.0, 7.6 Hz, 1H), 7.50 (d, J = 8.0 Hz, 1H), 3.86-3.67 |
| | | (m, 2H), 2.78-2.68 (m, 2H), 2.65-2.53 (m, 2H), 2.38 (s, 3H) |
| I-745 | 427.1 | 1H NMR (400 MHz, DMSO-d6) δ = 9.96 (s, 1H), 9.35 (d, J = |
| | | 2.0 Hz, 1H), 9.28 (d, J = 7.6 Hz, 1H), 8.89 (dd, J = 1.6, 4.8 Hz, |
| | | 1H), 8.59-8.54 (m, 1H), 8.47 (s, 1H), 8.16 (d, J = 1.6 Hz, 1H), |
| | | 7.91 (dd, J = 1.6, 8.0 Hz, 1H), 7.71 (dd, J = 4.8, 8.0 Hz, 1H), |
| | | 7.54 (d, J = 8.0 Hz, 1H), 7.17 (d, J = 2.4 Hz, 1H), 6.88 (dd, J = |
| | | 2.4, 7.6 Hz, 1H), 3.90 (s, 3H), 2.38 (s, 3H) |
| I-744 | 338 | 1H NMR (400 MHz, DMSO-d6) δ = 10.07 (s, 1H), 9.70 (s, 1H), |
| | | 9.48 (dd, J = 6.4, 7.2 Hz, 1H), 8.58 (s, 1H), 8.10 (d, J = 1.6 Hz, |
| | | 1H), 7.86 (dd, J = 1.6, 7.8 Hz, 1H), 7.68 (dd, J = 2.4, 9.8 Hz, |
| | | 1H), 7.51 (d, J = 8.0 Hz, 1H), 7.25-7.21 (m, 1H), 2.36 (s, 3H) |
| I-743 | 426.2 | 1H NMR (400 MHz, DMSO-d6) δ = 9.95 (s, 1H), 9.28 (d, J = |
| | | 7.6 Hz, 1H), 8.47 (s, 1H), 8.20 (d, J = 7.2 Hz, 2H), 8.15 (s, 1H), |
| | | 7.90 (d, J = 8.0 Hz, 1H), 7.74 (d, J = 7.2 Hz, 1H), 7.63-7.71 |
| | | (m, 2H), 7.53 (d, J = 8.0 Hz, 1H), 7.17 (d, J = 2.4 Hz, 1H), 6.88 |
| | | (dd, J = 2.4, 7.6 Hz, 1H), 3.90 (s, 3H), 2.38 (s, 3H) |
| I-742 | 394.1 | 1H NMR (400 MHz, DMSO-d6) δ = 9.89 (s, 1H), 9.26 (d, J = |
| | | 7.6 Hz, 1H), 8.45 (s, 1H), 8.07 (s, 1H), 7.81 (dd, J = 1.2, 8.0 |
| | | Hz, 1H), 7.49 (d, J = 8.0 Hz, 1H), 7.16 (d, J = 2.4 Hz, 1H), 6.87 |
| | | (dd, J = 2.4, 7.6 Hz, 1H), 6.16 (d, J = 5.6 Hz, 1H), 4.99-5.12 |
| | | (m, 1H), 3.90 (s, 3H), 2.36 (s, 3H), 1.54 (d, J = 6.8 Hz, 3H) |
| I-741 | 382.2 | 1H NMR (400 MHz, DMSO-d6) δ = 10.06 (s, 1H), 9.49 (dd, J = |
| | | 6.0, 7.2 Hz, 1H), 8.59 (s, 1H), 8.09 (d, J = 1.2 Hz, 1H), 7.83 |
| | | (dd, J = 1.6, 8.0 Hz, 1H), 7.69 (dd, J = 2.4, 10.0 Hz, 1H), 7.51 |
| | | (d, J = 8.0 Hz, 1H), 7.15-7 .30 (m, 1H), 6.17 (d, J = 6.0 Hz, |
| | | 1H), 4.99-5.14 (m, 1H), 2.37 (s, 3H), 1.55 (d, J = 7.2 Hz, 3H) |
| I-739 | 366.1 | 1H NMR (400 MHz, DMSO-d6) δ = 10.05 (s, 1H), 9.48 (dd, J = |
| | | 6.4, 7.6 Hz, 1H), 8.57 (s, 1H), 8.05 (s, 1H), 7.81 (dd, J = 1.6, |
| | | 8.0 Hz, 1H), 7.68 (dd, J = 2.4, 9.6 Hz, 1H), 7.49 (d, J = 8.0 Hz, |
| | | 1H), 7.25-7.15 (m, 1H), 3.05-2.95 (m, 2H), 2.35 (s, 3H), 1.34 (t, |
| | | J = 7.6 Hz, 3H) |
| I-738 | 397.1 | 1H NMR (400 MHz, DMSO-d6) δ = 10.31 (s, 1H), 9.54 (d, J = |
| | | 6.8 Hz, 1H), 9.34 (s, 1H), 8.89 (d, J = 4.0 Hz, 1H), 8.80 (s, 1H), |
| | | 8.55 (d, J = 7.6 Hz, 1H), 8.17 (s, 1H), 7.91 (dd, J = 8.4, 16.8 |
| | | Hz, 2H), 7.76-7.64 (m, 2H), 7.55 (d, J = 8.0 Hz, 1H), 7.32 (t, |
| | | J = 6.4 Hz, 1H), 2.40 (s, 3H) |
| I-737 | 378.2 | 1H NMR (400 MHz, DMSO-d6) δ = 9.89 (s, 1H), 9.27 (d, J = |
| | | 7.6 Hz, 1H), 8.45 (s, 1H), 8.06 (d, J = 1.2 Hz, 1H), 7.80 (dd, J = |
| | | 1.6, 8.0 Hz, 1H), 7.47 (d, J = 8.0 Hz, 1H), 7.16 (d, J = 2.4 Hz, |
| | | 1H), 6.87 (dd, J = 2.4, 7.6 Hz, 1H), 3.90 (s, 3H), 3.05-2.95 (m, |
| | | 2H), 2.35 (s, 3H), 1.34 (t, J = 7.6 Hz, 3H) |
| I-736 | 410.2 | 1H NMR (400 MHz, DMSO-d6) δ = 10.01 (s, 1H), 9.34 (d, J = |
| | | 7.2 Hz, 1H), 8.54 (s, 1H), 8.25-8.15 (m, 3H), 7.90 (dd, J = 1.6, |
| | | 7.6 Hz, 1H), 7.80-7.72 (m, 1H), 7.70-7.65 (m, 2H), 7.58 (d, J = |
| | | 0.8 Hz, 1H), 7.53 (d, J = 8.0 Hz, 1H), 7.04 (dd, J = 1.6, 7.2 |
| | | Hz, 1H), 2.43 (s, 3H), 2.38 (s, 3H) |
| I-735 | 408.2 | 1H NMR (400 MHz, DMSO-d6) δ = 10.01 (s, 1H), 9.41 (d, J = |
| | | 7.2 Hz, 1H), 8.57 (s, 1H), 8.10 (s, 1H), 7.82 (dd, J = 1.6, 8.0 |
| | | Hz, 1H), 7.67 (s, 1H), 7.49 (d, J = 8.0 Hz, 1H), 7.12 (dd, J = |
| | | 1.2, 7.2 Hz, 1H), 6.22-6.19 (m, 1H), 6.18-6.14 (m, 1H), 6.16 |
| | | (s, 1H), 5.11-4.99 (m, 1H), 4.54 (s, 2H), 3.36 (s, 3H), 2.37 (s, |
| | | 3H), 1.54 (d, J = 6.4 Hz, 2H), 1.57-1.51 (m, 1H) |
| I-734 | 392.1 | 1H NMR (400 MHz, DMSO-d6) δ = 9.77 (s, 1H), 9.22 (s, 1H), |
| | | 8.79 (s, 1H), 8.30-8.22 (m, 1H), 8.13 (d, J = 1.6 Hz, 1H), 8.00 |
| | | (dd, J = 1.2, 9.2 Hz, 1H), 7.85-7.75 (m, 2H), 7.48 (d, J = 8.0 |
| | | Hz, 1H), 6.93 (d, J = 2.4 Hz, 1H), 5.15-5.05 (m, 1H), 3.92 (s, |
| | | 3H), 3.70-3.60 (m, 1H), 3.40-3.35(m, 2H), 2.70-2.60 (m, |
| | | 2H), 2.37 (s, 3H) |
| I-733 | 440.1 | 1H NMR (400 MHz, DMSO-d6) δ = 10.07 (s, 1H), 9.42 (d, J = |
| | | 8.0 Hz, 1H), 8.59 (s, 1H), 8.13-8.25 (m, 3H), 7.92 (dd, J = 2.0, |
| | | 8.0 Hz, 1H), 7.71-7.78 (m, 1H), 7.64-7.71 (m, 3H), 7.54 (d, J = |
| | | 8.0 Hz, 1H), 7.12 (dd, J = 1.6, 7.2 Hz, 1H), 4.55 (s, 2H), 3.37 |
| | | (s, 3H), 2.39 (s, 3H) |
| I-732 | 441.1 | 1H NMR (400 MHz, DMSO-d6) δ = 9.36 (d, J = 7.2 Hz, 1H), |
| | | 9.28 (d, J = 1.6 Hz, 1H), 8.87-8.81 (m, 1H), 8.56-8.48 (m, |
| | | 2H), 8.13 (d, J = 1.2 Hz, 1H), 7.88 (dd, J = 1.2, 8.0 Hz, 1H), |
| | | 7.71-7.62 (m, 2H), 7.50 (d, J = 8.0 Hz, 1H), 7.09 (d, J = 7.2 |
| | | Hz, 1H), 4.51 (s, 2H), 3.34 (s, 3H), 2.34 (s, 3H) |
| I-731 | 434.2 | 1H NMR (400 MHz, DMSO-d6) δ = 9.71 (s, 1H), 8.84 (s, 1H), |
| | | 8.76 (s, 1H), 8.21 (d, J = 9.2 Hz, 1H), 8.10 (d, J = 1.2 Hz, 1H), |
| | | 7.81 (dd, J = 1.6, 8.0 Hz, 1H), 7.52 (dd, J = 0.8, 9.2 Hz, 1H), |
| | | 7.46 (d, J = 8.0 Hz, 1H), 5.44 (s, 1H), 4.62 (s, 2H), 3.49 (s, 2H), |
| | | 2.66 (s, 3H), 2.35 (s, 3H), 0.63-0.56 (m, 2H), 0.52-0.47 (m, |
| | | 2H) |
| I-730 | 444.2 | 1H NMR (400 MHz, DMSO-d6) δ = 9.77 (s, 1H), 9.22 (s, 1H), |
| | | 8.79 (s, 1H), 8.31-8.23 (m, 1H), 8.13 (d, J = 1.6 Hz, 1H), 8.01 |
| | | (dd, J = 1.6, 9.2 Hz, 1H), 7.86-7.75 (m, 2H), 7.48 (d, J = 8.0 |
| | | Hz, 1H), 6.93 (d, J = 2.4 Hz, 1H), 6.17 (d, J = 5.6 Hz, 1H), 5.06 |
| | | (quin, J = 6.4 Hz, 1H), 3.92 (s, 3H), 2.38 (s, 3H), 1.55 (d, J = |
| | | 6.8 Hz, 3H) |
| I-729 | 400.1 | 1H NMR (400 MHz, DMSO-d6) δ = 9.78 (s, 1H), 9.70 (s, 1H), |
| | | 9.21 (s, 1H), 8.78 (s, 1H), 8.26 (d, J = 9.2 Hz, 1H), 8.14 (d, J = |
| | | 1.6 Hz, 1H), 8.00 (dd, J = 1.6, 9.2 Hz, 1H), 7.91-7.73 (m, 2H), |
| | | 7.50 (d, J = 8.0 Hz, 1H), 6.92 (d, J = 2.4 Hz, 1H), 3.92 (s, 3H), |
| | | 2.37 (s, 3H) |
| I-727 | 470.2 | 1H NMR (400 MHz, DMSO-d6): δ ppm 10.02 (d, J = 7.6 Hz, |
| | | 1H), 9.43 (d, J = 7.2 Hz, 1H), 8.58 (s, 1H), 8.13-8.10 (m, 1H), |
| | | 8.08 (dd, J = 1.6, 5.6 Hz, 1H), 7.85-7.79 (m, 2H), 7.65 (dd, J = |
| | | 1.6, 7.2 Hz, 1H), 7.49 (d, J = 8.0 Hz, 1H), 7.00 (d, J = 2.4 Hz, |
| | | 1H), 5.40 (dd, J = 6.8, 10.8 Hz, 1H), 4.52-4.09 (m, 1H), 3.93 |
| | | (s, 3H), 3.78-3.68 (m, 0.5H), 3.30-3.25 (m, 0.5H), 2.74-2.66 |
| | | (m, 1H), 2.64-2.56 (m, 1H), 2.42-2.39 (m, 0.7H), 2.37 (s, |
| | | 3H), 2.35-2.30 (m, 0.3H), 2.29-2.18 (m, 1H) |
| I-726 | 411.1 | 1H NMR (400 MHz, DMSO-d6) δ = 9.99 (s, 1H), 9.40-9.25 |
| | | (m, 2H), 8.88 (dd, J = 1.6, 4.8 Hz, 1H), 8.60-8.50 (m, 2H), |
| | | 8.17 (d, J = 1.6 Hz, 1H), 7.91 (dd, J = 1.6, 8.0 Hz, 1H), 7.75- |
| | | 7.65 (m, 1H), 7.60-7.49 (m, 2H), 7.02 (dd, J = 1.6, 7.2 Hz, 1H), |
| | | 2.42 (s, 3H), 2.38 (s, 3H) |
| I-725 | 438.1 | 1H NMR (400 MHz, DMSO-d6) δ = 10.10 (s, 1H), 9.43 (d, J = |
| | | 7.6 Hz, 1H), 8.60 (s, 1H), 8.06 (d, J = 1.6 Hz, 1H), 7.99 (d, J = |
| | | 2.0 Hz, 1H), 7.82 (dd, J = 1.6, 8.0 Hz, 1H), 7.50 (d, J = 8.0 Hz, |
| | | 1H), 7.26 (dd, J = 2.4, 7.6 Hz, 1H), 4.31 (dd, J = 5.2, 7.6 Hz, |
| | | 1H), 3.72-3.83 (m, 1H), 3.58-3.68 (m, 1H), 3.12-3.27 (m, |
| | | 2H), 2.36 (s, 3H), 2.05-2.13 (m, 1H), 1.79-1.91 (m, 2H), 1.62- |
| | | 1.72 (m, 1H) |
| I-724 | 438.2 | 1H NMR (400 MHz, DMSO-d6) δ = 10.10 (s, 1H), 9.43 (dd, J = |
| | | 0.8, 7.6, 1H), 8.60 (s, 1H), 8.06 (d, J = 1.6 Hz, 1H), 7.98 (d, |
| | | J = 1.6 Hz, 1H), 7.81 (dd, J = 2.0, 8.0, 1H), 7.49 (d, J = 8.0 Hz, |
| | | 1H), 7.26 (dd, J = 2.4, 7.6 Hz, 1H), 4.25-4.36 (m, 1H), 3.72- |
| | | 3.81 (m, 1H), 3.60-3.66 (m, 1H), 3.11-3.27 (m, 2H), 2.36 (s, |
| | | 3H), 2.02-2.15 (m, 1H), 1.80-1.94 (m, 2H), 1.61-1.72 (m, |
| | | 1H) |
| I-723 | 477.2 | 1H NMR (400 MHz, DMSO-d6) δ = 9.79 (s, 1H), 9.36 (d, J = |
| | | 1.6 Hz, 1H), 9.23 (s, 1H), 8.93-8.86 (m, 1H), 8.80 (s, 1H), |
| | | 8.61-8.55 (m, 1H), 8.26 (s, 1H), 8.21 (d, J = 1.6 Hz, 1H), |
| | | 8.04-7.99 (m, 1H), 7.94-7.87 (m, 1H), 7.81 (d, J = 2.4 Hz, 1H), |
| | | 7.74-7.68 (m, 1H), 7.53 (d, J = 8.0 Hz, 1H), 6.93 (d, J = 2.4 |
| | | Hz, 1H), 3.92 (s, 3H), 2.39 (s, 3H) |
| I-722 | 428.2 | 1H NMR (400 MHz, DMSO-d6) δ = 9.77 (s, 1H), 9.23 (s, 1H), |
| | | 8.79 (s, 1H), 8.33-8.24 (m, 1H), 8.11 (d, J = 1.2 Hz, 1H), |
| | | 8.05-7.97 (m, 1H), 7.85-7.75 (m, 2H), 7.48 (d, J = 7.6 Hz, 1H), |
| | | 6.93 (d, J = 2.4 Hz, 1H), 3.92 (s, 3H), 3.05-2.97 (m, 2H), 2.37 |
| | | (s, 3H) |
| I-721 | 447.2 | 1H NMR (400 MHz, DMSO-d6) δ = 10.06 (s, 1H), 9.48 (dd, J = |
| | | 0.8, 7.2 Hz, 1H), 9.30 (d, J = 2.0 Hz, 1H), 8.63 (s, 1H), 8.54 |
| | | (d, J = 2.0 Hz, 1H), 8.36 (d, J = 0.8 Hz, 1H), 8.11 (d, J = 1.6 Hz, |
| | | 1H), 7.86-7.80 (m, 2H), 7.51 (d, J = 8.0 Hz, 1H), 6.16 (d, J = |
| | | 5.6 Hz, 1H), 5.10-5.02 (m, 1H), 2.38 (s, 3H), 1.55 (d, J = 6.4 |
| | | Hz, 3H) |
| I-720 | 431.2 | 1H NMR (400 MHz, DMSO-d6) δ = 10.06 (s, 1H), 9.48 (dd, J = |
| | | 0.8, 7.2 Hz, 1H), 9.30 (d, J = 2.0 Hz, 1H), 8.62 (s, 1H), 8.54 |
| | | (d, J = 2.0 Hz, 1H), 8.36 (d, J = 0.8 Hz, 1H), 8.09 (d, J = 1.6 Hz, |
| | | 1H), 7.81 (t, J = 1.6 Hz, 1H), 7.85-7.79 (m, 1H), 7.50 (d, J = |
| | | 8.0 Hz, 1H), 3.06-2.98 (m, 2H), 2.38 (s, 3H), 1.35 (t, J = 7.6 |
| | | Hz, 3H) |
| I-719 | 470.2 | 1H NMR (400 MHz, DMSO-d6) δ = 10.07 (s, 1H), 9.42 (d, J = |
| | | 7.2 Hz, 1H), 8.60 (s, 1H), 8.13-8.25 (m, 3H), 7.92 (dd, J = 1.6, |
| | | 8.0 Hz, 1H), 7.71-7.78 (m, 2H), 7.65-7.70 (m, 2H), 7.54 (d, J = |
| | | 8.0 Hz, 1H), 7.15 (dd, J = 1.6, 7.6 Hz, 1H), 4.64 (s, 2H), |
| | | 3.53-3.61 (m, 4H), 2.39 (s, 3H) |
| I-718 | 363.1 | 1H NMR (400 MHz, DMSO-d6) δ = 9.71 (s, 2H), 9.15 (d, J = |
| | | 7.6 Hz, 1H), 8.36 (s, 1H), 8.12 (d, J = 1.6 Hz, 1H), 7.86-7.79 |
| | | (m, 1H), 7.49 (d, J = 8.0 Hz, 1H), 6.95-6.80 (m, 1H), 6.60 |
| | | (d, J = 2.4 Hz, 1H), 3.04 (s, 6H), 2.37 (s, 3H) |
| I-717 | 405.1 | 1H NMR (400 MHz, DMSO-d6) δ = 9.79 (s, 1H), 9.70 (s, 1H), |
| | | 9.18 (d, J = 8.0 Hz, 1H), 8.40 (s, 1H), 8.11 (d, J = 1.6 Hz, 1H), |
| | | 7.83 (m, 1H), 7.49 (d, J = 8.0 Hz, 1H), 7.09 (m, 1H), 6.90 (d, |
| | | J = 2.4 Hz, 1H), 3.82-3.69 (m, 4H), 3.31-3.28 (m, 4H), 2.36 (s, |
| | | 3H) |
| I-716 | 470.2 | 1H NMR (400 MHz, DMSO-d6): δ ppm 10.01 (s, 1H), 9.43 (d, |
| | | J = 7.2 Hz, 1H), 8.58 (s, 1H), 8.13-8.07 (m, 2H), 7.85-7.78 |
| | | (m, 2H), 7.65 (dd, J = 1.6, 7.2 Hz, 1H), 7.49 (d, J = 8.0 Hz, 1H), |
| | | 6.99 (d, J = 2.4 Hz, 1H), 5.40 (d, J = 7.2 Hz, 1H), 4.21-4.09 |
| | | (m, 1H), 3.93 (s, 3H), 3.32-3.28 (m, 1H), 2.75-2.65 (m, 2H), |
| | | 2.37 (s, 3H), 2.29-2.19 (m, 2H). |
| I-715 | 470.3 | 1H NMR (400 MHz, DMSO-d6): δ ppm 10.03 (s, 1H), 9.43 (d, |
| | | J = 7.2 Hz, 1H), 8.58 (s, 1H), 8.13-8.06 (m, 2H), 7.85-7.80 |
| | | (m, 2H), 7.65 (dd, J = 1.6, 7.2 Hz, 1H), 7.49 (d, J = 8.0 Hz, 1H), |
| | | 6.99 (d, J = 2.4 Hz, 1H), 5.38 (d, J = 6.2 Hz, 1H), 4.52-4.41 |
| | | (m, 1H), 3.93 (s, 3H), 3.78-3.69 (m, 1H), 2.63-2.56 (m, 2H), |
| | | 2.45-2.38 (m, 2H), 2.37 (s, 3H) |
| I-714 | 458.1 | 1H NMR (400 MHz, DMSO-d6) δ = 10.06 (s, 1H), 9.49 (d, J = |
| | | 7.6 Hz, 1H), 9.30 (d, J = 1.6 Hz, 1H), 8.63 (s, 1H), 8.54 (d, J = |
| | | 2.0 Hz, 1H), 8.36 (s, 1H), 8.12 (d, J = 1.2 Hz, 1H), 7.78-7.86 |
| | | (m, 2H), 7.50 (d, J = 8.0 Hz, 1H), 4.97-5.30 (m, 1H), 3.59- |
| | | 3.73 (m, 1H), 3.48 (s, 1H), 2.63-2.68 (m, 2H), 2.38 (s, 3H) |
| I-713 | 484.2 | 1H NMR (400 MHz, DMSO-d6) δ = 9.96 (s, 1H), 9.38 (d, J = |
| | | 7.2 Hz, 1H), 8.55 (s, 1H), 8.08 (s, 1H), 8.01 (s, 1H), 7.90 (s, |
| | | 1H), 7.80 (dd, J = 1.6, 8.0 Hz, 1H), 7.74 (s, 1H), 7.57 (dd, J = |
| | | 1.6, 7.2 Hz, 1H), 7.49 (d, J = 8.0 Hz, 1H), 4.36-4.26 (m, 1H), |
| | | 3.81-3.71 (m, 4H), 3.67-3.60 (m, 1H), 3.25-3.13 (m, 2H), |
| | | 2.37 (s, 3H), 2.15-2.04 (m, 1H), 1.94-1.78 (m, 2H), 1.72- |
| | | 1.61 (m, 1H) |
| I-712 | 403.1 | 1H NMR (400 MHz, DMSO-d6) δ = 9.77-9.72 (m, 1H), 9.70 |
| | | (s, 1H), 9.13 (d, J = 8.0 Hz, 1H), 8.40-8.33 (m, 1H), 8.11 |
| | | (d, J = 1.6 Hz, 1H), 7.82 (dd, J = 1.6, 8.0 Hz, 1H), 7.49 (d, J = |
| | | 8.0 Hz, 1H), 7.05 (dd, J = 2.8, 7.6 Hz, 1H), 6.83 (d, J = 2.0 Hz, |
| | | 1H), 3.38-3.34 (m, 4H), 2.36 (s, 3H), 1.61 (s, 6H) |
| I-711 | 412.1 | 1H NMR (400 MHz, DMSO-d6) δ = 10.90 (s, 1H), 9.45 (d, J = |
| | | 6.8 Hz, 1H), 8.66 (s, 1H), 7.90-7.95 (m, 1H), 7.84-7.89 (m, |
| | | 1H), 7.79 (d, J = 9.2 Hz, 1H), 7.50-7.57 (m, 1H), 7.19 (t, J = |
| | | 6.4 Hz, 1H), 4.75 (t, J = 12.4 Hz, 4H), 2.30 (s, 3H). |
| I-710 | 505.3 | 1H NMR (400 MHz, DMSO-d6) δ = 9.88 (s, 1H), 9.26 (d, J = |
| | | 7.6 Hz, 1H), 8.44 (s, 1H), 8.06 (s, 1H), 7.79 (dd, J = 1.6, 8.0 Hz, |
| | | 1H), 7.47 (d, J = 8.0 Hz, 1H), 7.13 (d, J = 2.4 Hz, 1H), 6.85 (dd, |
| | | J = 2.4, 7.6 Hz, 1H), 5.10-5.01 (m, 1H), 4.20-4.11 (m, 3H), |
| | | 3.12-2.95 (m, 2H), 2.56-2.52 (m, 2H), 2.47-2.41 (m, 4H), |
| | | 2.35 (s, 3H), 1.99-1.84 (m, 2H), 1.78-1.61 (m, 4H), 1.21 (d, |
| | | J = 6.0 Hz, 3H) |
| I-709 | 519.3 | 1H NMR (400 MHz, DMSO-d6) δ = 9.87 (s, 1H), 9.26 (d, J = |
| | | 7.8 Hz, 1H), 8.45 (s, 1H), 8.06 (d, J = 1.6 Hz, 1H), 7.80 (dd, J = |
| | | 1.6, 8.0 Hz, 1H), 7.48 (d, J = 8.0 Hz, 1H), 7.14 (d, J = 2.4 Hz, |
| | | 1H), 6.86 (dd, J = 2.4, 7.6 Hz, 1H), 4.99 (d, J = 5.6 Hz, 1H), |
| | | 4.16 (t, J = 6.4 Hz, 2H), 3.98-3.86 (m, 1H), 3.15-3.08 (m, |
| | | 1H), 3.02-2.95 (m, 1H), 2.53 (d, J = 7.2 Hz, 2H), 2.46-2.42 |
| | | (m, 4H), 2.35 (s, 3H), 1.98-1.89 (m, 2H), 1.72-1.65 (m, 4H), |
| | | 1.57-1.42 (m, 2H), 0.92 (t, J = 7.2 Hz, 3H) |
| I-708 | 471.3 | 1H NMR (400 MHz, DMSO-d6 + D20) 9.41 (d, J = 7.2 Hz, 1H), |
| | | 9.34 (d, J = 2.0 Hz, 1H), 8.89 (d, J = 1.6 Hz, 1H), 8.65-8.51 |
| | | (m, 2H), 8.18 (d, J = 1.6 Hz, 1H), 7.92 (dd, J = 1.6, 8.0 Hz, 1H), |
| | | 7.77-7.67 (m, 2H), 7.54 (d, J = 8.0 Hz, 1H), 7.14 (dd, J = 1.2, |
| | | 7.2 Hz, 1H), 4.63 (s, 2H), 3.61-3.56 (m, 2H), 3.56-3.52 (m, |
| | | 2H), 2.39 (s, 3H) |
| I-707 | 485.2 | 1H NMR (400 MHz, DMSO-d6) δ = 10.02 (s, 1H), 9.43 (d, J = |
| | | 7.6 Hz, 1H), 8.58 (s, 1H), 8.11 (d, J = 0.8 Hz, 1H), 7.96 (d, J = |
| | | 1.6 Hz, 1H), 7.83 (d, J = 2.4 Hz, 1H), 7.77-7.70 (m, 1H), 7.67- |
| | | 7.62 (m, 1H), 7.44 (d, J = 8.0 Hz, 1H), 7.00 (d, J = 2.4 Hz, |
| | | 1H), 5.15 (s, 1H), 4.42 (s, 1H), 3.94 (s, 3H), 3.69-3.60 (m, |
| | | 3H), 3.45 (d, J = 10.4 Hz, 1H), 2.35 (s, 3H), 2.11-1.88 (m, 2H) |
| I-706 | 363.3 | 1H NMR (400 MHz, DMSO-d6): δ ppm 9.69 (s, 1H), 9.65 (s, |
| | | 1H), 9.03 (d, J = 7.6 Hz, 1H), 8.28 (s, 1H), 8.11 (d, J = 1.6 Hz, |
| | | 1H), 7.81 (dd, J = 1.6, 8.0 Hz, 1H), 7.48 (d, J = 8.0 Hz, 1H), |
| | | 6.62-6.55 (m, 2H), 6.39 (d, J = 2.0 Hz, 1H), 3.17-3.07 (m, |
| | | 2H), 2.35 (s, 3H), 1.21 (t, J = 7.2 Hz, 3H). |
| I-703 | 487.1 | 1H NMR (400 MHz, DMSO-d6) δ = 10.06 (s, 1H), 9.48 (d, J = |
| | | 7.2 Hz, 1H), 9.29 (d, J = 1.6 Hz, 1H), 8.62 (s, 1H), 8.53 (d, J = |
| | | 1.6 Hz, 1H), 8.35 (s, 1H), 8.09 (s, 1H), 7.82 (d, J = 7.6 Hz, 2H), |
| | | 7.50 (d, J = 8.0 Hz, 1H), 4.31 (dd, J = 5.2, 7.2 Hz, 1H), 3.74- |
| | | 3.81 (m, 1H), 3.58-3.69 (m, 1H), 3.14-3.23 (m, 2H), 2.38 (s, |
| | | 3H), 2.10 (dd, J = 5.6, 13.6 Hz, 1H), 1.79-1.93 (m, 2H), 1.62- |
| | | 1.73 (m, 1H) |
| I-754 | 422.2 | 1H NMR (400 MHz, DMSO-d6) δ = 10.00 (s, 1H), 9.40 (d, J = |
| | | 7.2 Hz, 1H), 8.57 (s, 1H), 8.08 (d, J = 1.6 Hz, 1H), 7.81 (dd, J = |
| | | 1.6, 8.0 Hz, 1H), 7.72 (s, 1H), 7.49 (d, J = 8.0 Hz, 1H), 7.13 |
| | | (dd, J = 1.2, 7.2 Hz, 1H), 4.77-4.69 (m, 1H), 4.63 (s, 2H), 3.63- |
| | | 3.52 (m, 4H), 3.08-2.97 (m, 2H), 2.36 (s, 3H), 1.39-1.28 (m, |
| | | 3H) |
| I-700 | 394.2 | 1H NMR (400 MHz, DMSO-d6) δ = 10.02 (s, 1H), 9.70 (s, |
| | | 1H), 9.40 (d, J = 7.2 Hz, 1H), 8.57 (s, 1H), 8.12 (d, J = 1.6 Hz, |
| | | 1H), 7.84-7.86 (m, 1H), 7.72 (s, 1H), 7.51 (d, J = 8.0 Hz, 1H), |
| | | 7.12-7.14 (m, 1H), 4.72 (t, J = 5.6 Hz, 1H), 4.63 (s, 2H), 3.57- |
| | | 3.64 (m, 2H), 3.52-3.57 (m, 2H), 2.37 (s, 3H) |
| I-699 | 439.1 | 1H NMR (400 MHz, DMSO-d6) δ = 10.63 (s, 1H), 9.98 (d, J = |
| | | 7.2 Hz, 1H), 9.13 (s, 1H), 8.52 (d, J = 1.6 Hz, 1H), 8.47 (s, 1H), |
| | | 8.30-8.25 (m, 1H), 7.98 (d, J = 7.6 Hz, 1H), 7.85-7.75 (m, |
| | | 1H), 5.67 (d, J = 3.6 Hz, 1H), 5.01-4.90 (m, 1H), 4.24-4.12 |
| | | (m, 3H), 4.04-3.92 (m, 1H), 2.88 (s, 3H), 2.64-2.46 (m, 2H) |
| I-698 | 485.3 | 1H NMR (400 MHz, DMSO-d6) δ = 10.02 (s, 1H), 9.51 (d, J = |
| | | 3.2 Hz, 1H), 8.58 (s, 1H), 8.11 (s, 1H), 7.95 (d, J = 1.6 Hz, 1H), |
| | | 7.83 (d, J = 2.3 Hz, 1H), 7.77-7.71 (m, 1H), 7.69-7.63 (m, |
| | | 1H), 7.48-7.34 (m, 1H), 7.04-6.91 (m, 1H), 5.15 (d, J = 3.6 |
| | | Hz, 1H), 4.42 (s, 1H), 3.94 (s, 3H), 3.68-3.58 (m, 3H), 3.48- |
| | | 3.41 (m, 1H), 2.35 (s, 3H), 2.10-1.90 (m, 2H) |
| I-697 | 439.1 | 1H NMR (400 MHz, DMSO-d6) δ = 10.10 (s, 1H), 9.44 (d, J = |
| | | 7.6 Hz, 1H), 8.60 (s, 1H), 8.02-7.89 (m, 2H), 7.77-7.63 (m, |
| | | 1H), 7.45 (d, J = 8.0 Hz, 1H), 7.32-7.17 (m, 1H), 5.14 (d, J = |
| | | 3.6 Hz, 1H), 4.42 (s, 1H), 3.71-3.59 (m, 3H), 3.45 (d, J = 10.8 |
| | | Hz, 1H), 2.34 (s, 3H), 2.13-1.91 (m, 2H) |
| I-696 | 469.3 | 1H NMR (400 MHz, DMSO-d6) δ = 9.76 (s, 1H), 9.21 (s, 1H), |
| | | 8.78 (s, 1H), 8.26 (d, J = 9.2 Hz, 1H), 8.09 (s, 1H), 8.00 (d, J = |
| | | 9.6 Hz, 1H), 7.84-7.74 (m, 2H), 7.47 (d, J = 8.0 Hz, 1H), 6.92 |
| | | (d, J = 2.0 Hz, 1H), 4.52 (dd, J = 5.6, 8.0 Hz, 1H), 3.91 (s, 3H), |
| | | 3.65-3.40(m, 1H), 3.00-2.85 (m, 2H), 2.36 (s, 3H), 2.25- |
| | | 2.14 (m, 1H), 2.10-1.97 (m, 1H), 1.89-1.82 (m, 1H), 1.80- |
| | | 1.71 (m, 1H) |
| I-695 | 447.2 | 1H NMR (400 MHz, DMSO-d6) δ = 9.79 (s, 1H), 9.38 (s, 1H), |
| | | 9.29 (d, J = 2.0 Hz, 1H), 8.83 (s, 1H), 8.41 (d, J = 2.0 Hz, 1H), |
| | | 8.31 (d, J = 9.2 Hz, 1H), 8.19-8.10 (m, 2H), 7.80 (dd, J = 1.6, |
| | | 8.0 Hz, 1H), 7.49 (d, J = 8.0 Hz, 1H), 6.16 (d, J = 5.6 Hz, 1H), |
| | | 5.10-5.01 (m, 1H), 2.38 (s, 3H), 1.55 (d, J = 6.8 Hz, 3H) |
| I-694 | 403.1 | 1H NMR (400 MHz, DMSO-d6) δ = 10.08 (s, 1H), 9.72 (s, |
| | | 1H), 9.49 (d, J = 7.2 Hz, 1H), 9.30 (d, J = 1.6 Hz, 1H), 8.64 (s, |
| | | 1H), 8.55 (d, J = 1.6 Hz, 1H), 8.37 (s, 1H), 8.14 (d, J = 1.2 Hz, |
| | | 1H), 7.90-7.80 (m, 2H), 7.53 (d, J = 8.0 Hz, 1H), 2.39 (s, 3H) |
| I-693 | 484.2 | 1H NMR (400 MHz, DMSO-d6) δ = 9.97 (s, 1H), 9.39 (d, J = |
| | | 7.2 Hz, 1H), 8.55 (s, 1H), 8.08 (d, J = 1.2 Hz, 1H), 8.01 (s, 1H), |
| | | 7.91 (s, 1H), 7.80 (dd, J = 1.6, 8.0 Hz, 1H), 7.75 (s, 1H), 7.57 |
| | | (dd, J = 1.6, 7.2 Hz, 1H), 7.49 (d, J = 8.0 Hz, 1H), 4.35-4.26 |
| | | (m, 1H), 3.81-3.70 (m, 4H), 3.67-3.59 (m, 1H), 3.28-3.12 |
| | | (m, 2H), 2.37 (s, 3H), 2.15-2.04 (m, 1H), 1.92-1.80 (m, 2H), |
| | | 1.71-1.61 (m, 1H) |
| I-692 | 484.2 | 1H NMR (400 MHz, DMSO-d6) δ = 9.97 (s, 1H), 9.39 (d, J = |
| | | 7.2 Hz, 1H), 8.55 (s, 1H), 8.09 (d, J = 1.6 Hz, 1H), 8.01 (s, 1H), |
| | | 7.91 (s, 1H), 7.80 (dd, J = 1.6, 8.0 Hz, 1H), 7.75 (s, 1H), 7.57 |
| | | (dd, J = 1.6, 7.2 Hz, 1H), 7.49 (d, J = 8.0 Hz, 1H), 4.36-4.27 |
| | | (m, 1H), 3.81-3.71 (m, 4H), 3.67-3.60 (m, 1H), 3.28-3.12 |
| | | (m, 2H), 2.37 (s, 3H), 2.15-2.04 (m, 1H), 1.93-1.79 (m, 2H), |
| | | 1.72-1.61 (m, 1H) |
| I-691 | 455.2 | 1H NMR (400 MHz, DMSO-d6) δ = 9.77 (s, 1H), 9.22 (s, 1H), |
| | | 8.79 (s, 1H), 8.30-8.22 (m, 1H), 8.13 (d, J = 1.6 Hz, 1H), 8.00 |
| | | (dd, J = 1.6, 9.2 Hz, 1H), 7.84-7.75 (m, 2H), 7.48 (d, J = 8.0 |
| | | Hz, 1H), 6.93 (d, J = 2.4 Hz, 1H), 5.11 (t, J = 7.6 Hz, 1H), 3.92 |
| | | (s, 3H), 3.71-3.59 (m, 1H), 3.40-3.35 (m, 2H), 2.71-2.60 (m, |
| | | 2H), 2.37 (s, 3H) |
| I-690 | 439.1 | 1H NMR (400 MHz, DMSO-d6): δ ppm 10.10 (s, 1H), 9.43 |
| | | (dd, J = 0.8, 7.6 Hz, 1H), 8.59 (s, 1H), 8.04-7.88 (m, 2H), 7.73 |
| | | (dd, J = 1.6, 8.0 Hz, 1H), 7.43 (d, J = 8.0 Hz, 1H), 7.26 (dd, J = |
| | | 2.4, 7.6 Hz, 1H), 5.14 (d, J = 3.6 Hz, 1H), 4.41 (s, 1H), 3.69- |
| | | 3.57 (m, 3H), 3.44 (d, J = 11.2 Hz, 1H), 2.33 (s, 3H), 2.11- |
| | | 1.99 (m, 1H), 1.97-1.87 (m, 1H) |
| I-689 | 485.3 | 1H NMR (400 MHz, DMSO-d6): δ ppm 10.01 (s, 1H), 9.45- |
| | | 9.41 (m, 1H), 8.57 (s, 1H), 8.11 (s, 1H), 7.95 (d, J = 1.6 Hz, |
| | | 1H), 7.83 (d, J = 2.4 Hz, 1H), 7.72 (dd, J = 1.6, 8.0 Hz, 1H), |
| | | 7.65 (dd, J = 1.6, 7.2 Hz, 1H), 7.43 (d, J = 8.4 Hz, 1H), 6.99 (d, |
| | | J = 2.4 Hz, 1H), 5.14 (d, J = 3.2 Hz, 1H), 4.41 (s, 1H), 3.93 (s, |
| | | 3H), 3.70-3.57 (m, 3H), 3.44 (d, J = 11.2 Hz, 1H), 2.34 (s, |
| | | 3H), 2.10-2.00 (m, 1H), 1.98-1.88 (m, 1H) |
| I-688 | 519.3 | 1H NMR (400 MHz, DMSO-d6) δ = 9.89 (s, 1H), 9.26 (d, J = |
| | | 7.6 Hz, 1H), 8.45 (s, 1H), 8.06 (d, J = 1.6 Hz, 1H), 7.80 (dd, J = |
| | | 1.6, 8.0 Hz, 1H), 7.48 (d, J = 8.0 Hz, 1H), 7.14 (d, J = 2.4 Hz, |
| | | 1H), 6.86 (dd, J = 2.8, 7.6 Hz, 1H), 5.00 (d, J = 5.6 Hz, 1H), |
| | | 4.15 (t, J = 6.0 Hz, 2H), 3.96-3.84 (m, 1H), 3.12 (dd, J = 4.0, |
| | | 14.8 Hz, 1H), 3.04-2.93 (m, 1H), 2.54 (t, J = 7.2 Hz, 2H), 2.47- |
| | | 2.41 (m, 4H), 2.37-2.33 (m, 1H), 2.35 (s, 2H), 2.00-1.90 (m, |
| | | 2H), 1.73-1.64 (m, 4H), 1.58-1.43 (m, 2H), 0.92 (t, J = 7.2 |
| | | Hz, 3H) |
| I-687 | 390.1 | 1H NMR (400 MHz, DMSO-d6) δ = 9.99 (s, 1H), 9.44 (d, J = |
| | | 7.2 Hz, 1H), 8.57 (s, 1H), 8.08 (d, J = 1.6 Hz, 1H), 7.80 (dd, J = |
| | | 2.0, 8.0 Hz, 1H), 7.73 (s, 1H), 7.49 (d, J = 8.0 Hz, 1H), 7.29 |
| | | (dd, J = 2.0, 7.2 Hz, 1H), 4.98 (dd, J = 6.0, 8.4 Hz, 2H), 4.70 (t, |
| | | J = 6.4 Hz, 2H), 4.33-4.46 (m, 1H), 2.66 (s, 3H), 2.36 (s, 3H) |
| I-686 | 505.3 | 1H NMR (400 MHz, DMSO-d6) δ = 9.90 (s, 1H), 9.27 (d, J = |
| | | 7.6 Hz, 1H), 8.40 (d, J = 3.2 Hz, 1H), 8.07 (d, J = 1.2 Hz, 1H), |
| | | 7.85-7.76 (m, 1H), 7.49 (d, J = 8.0 Hz, 1H), 7.15 (d, J = 2.4 |
| | | Hz, 1H), 6.91-6.67 (m, 1H), 5.11-5.00 (m, 1H), 4.22-4.12 |
| | | (m, 3H), 3.16-3.00 (m, 2H), 2.57-2.53 (m, 2H), 2.46-2.44 |
| | | (m, 4H), 2.36 (s, 3H), 1.99-1.90 (m, 2H), 1.73-1.65 (m, 4H), |
| | | 1.22 (d, J = 6.4 Hz, 3H) |
| I-685 | 433.1 | 1H NMR (400 MHz, DMSO-d6) δ = 10.33 (s, 1H), 9.72 (s, |
| | | 1H), 9.54 (d, J = 7.2 Hz, 1H), 8.83 (s, 1H), 8.51 (s, 1H), 8.36 (s, |
| | | 1H), 8.13 (d, J = 1.6 Hz, 1H), 7.95-7.85 (m, 2H), 7.53 (d, J = |
| | | 8.0 Hz, 1H), 4.85 (s, 2H), 2.39 (s, 3H) |
| I-684 | 400.1 | 1H NMR (400 MHz, DMSO-d6) δ = 10.04 (s, 1H), 9.71 (s, |
| | | 1H), 9.46-9.38 (m, 1H), 8.63-8.56 (m, 1H), 8.17-8.07 (m, |
| | | 2H), 7.91-7.78 (m, 2H), 7.65 (dd, J = 1.6, 7.2 Hz, 1H), 7.51 (d, |
| | | J = 8.0 Hz, 1H), 6.99 (d, J = 2.4 Hz, 1H), 3.93 (s, 3H), 2.38 (s, |
| | | 3H) |
| I-683 | 407.3 | 1H NMR (400 MHz, DMSO-d6) δ = 10.05 (s, 1H), 9.48 (dd, J = |
| | | 6.4, 7.2 Hz, 1H), 8.58 (s, 1H), 8.07 (d, J = 1.2 Hz, 1H), 7.82 |
| | | (dd, J = 1.6, 8.0 Hz, 1H), 7.68 (dd, J = 2.4, 9.8 Hz, 1H), 7.49 (d, |
| | | J = 8.0 Hz, 1H), 7.23 (dt, J = 2.4, 7.6 Hz, 1H), 4.95 (t, J = 8.0 |
| | | Hz, 1H), 4.02-3.86 (m, 1H), 3.75-3.44 (m, 1H), 2.75-2.68 |
| | | (m, 1H), 2.36 (s, 3H), 2.24-2.17 (m, 1H), 1.16 (d, J = 6.0 Hz, |
| | | 3H) |
| I-682 | 398.2 | 1H NMR (400 MHz, DMSO-d6) δ = 10.12 (s, 1H), 9.54 (d, J = |
| | | 7.2 Hz, 1H), 8.68 (s, 1H), 8.09 (d, J = 1.6 Hz, 1H), 7.99 (s, 1H), |
| | | 7.82 (m, 1H), 7.50 (d, J = 8.0 Hz, 1H), 7.35 (m, 1H), 2.67 (s, |
| | | 3H), 2.37 (s, 3H), 2.08 (t, J = 19.2 Hz, 3H) |
| I-681 | 472.3 | 1H NMR (400 MHz, DMSO-d6) δ = 10.00 (s, 1H), 9.42 (d, J = |
| | | 7.2 Hz, 1H), 8.58 (s, 1H), 8.10 (dd, J = 1.2, 4.4 Hz, 2H), 7.77- |
| | | 7.85 (m, 2H), 7.65 (dd, J = 1.6, 7.2 Hz, 1H), 7.49 (d, J = 8.0 Hz, |
| | | 1H), 6.99 (d, J = 2.4 Hz, 1H), 5.00 (d, J = 5.6 Hz, 1H), 3.88- |
| | | 3.97 (m, 4H), 3.12 (dd, J = 4.4, 14.8 Hz, 1H), 2.95-3.03 (m, |
| | | 1H), 2.37 (s, 3H), 1.43-1.60 (m, 2H), 0.92 (t, J = 7.2 Hz, 3H) |
| I-680 | 475.2 | 1H NMR (400 MHz, DMSO-d6) δ = 10.05 (s, 1H), 9.48 (d, J = |
| | | 7.2 Hz, 1H), 9.30 (d, J = 2.0 Hz, 1H), 8.63 (s, 1H), 8.54 (d, J = |
| | | 1.6 Hz, 1H), 8.36 (d, J = 0.8 Hz, 1H), 8.10 (d, J = 1.6 Hz, 1H), |
| | | 7.79-7.88 (m, 2H), 7.50 (d, J = 8.0 Hz, 1H), 5.00 (d, J = 5.6 |
| | | Hz, 1H), 3.85-3.98 (m, 1H), 3.12 (dd, J = 4.4, 14.8 Hz, 1H), |
| | | 2.94-3.05 (m, 1H), 2.38 (s, 3H), 1.42-1.61 (m, 2H), 0.92 (t, J = |
| | | 7.2 Hz, 3H) |
| I-679 | 487.2 | 1H NMR (400 MHz, DMSO-d6) δ = 10.06 (s, 1H), 9.49 (dd, J = |
| | | 0.8, 7.2 Hz, 1H), 9.30 (d, J = 2.0 Hz, 1H), 8.63 (s, 1H), 8.54 |
| | | (d, J = 2.0 Hz, 1H), 8.36 (d, J = 0.8 Hz, 1H), 8.09 (d, J = 1.6 Hz, |
| | | 1H), 7.80-7.83 (m, 2H), 7.50 (d, J = 8.1 Hz, 1H), 4.27-4.37 |
| | | (m, 1H), 3.74-3.83 (m, 1H), 3.61-3.66 (m, 1H), 3.12-3.24 |
| | | (m, 2H), 2.38 (s, 3H), 2.04-2.16 (m, 1H), 1.79-1.95 (m, 2H), |
| | | 1.60-1.74 (m, 1H) |
| I-678 | 487.1 | 1H NMR (400 MHz, DMSO-d6) δ = 10.06 (s, 1H), 9.49 (d, J = |
| | | 7.2 Hz, 1H), 9.30 (d, J = 2.0 Hz, 1H), 8.63 (s, 1H), 8.54 (d, J = |
| | | 2.0 Hz, 1H), 8.36 (s, 1H), 8.09 (d, J = 1.6 Hz, 1H), 7.80-7.83 |
| | | (m, 2H), 7.50 (d, J = 8.0 Hz, 1H), 4.25-4.37 (m, 1H), 3.74- |
| | | 3.83 (m, 1H), 3.60-3.67 (m, 1H), 3.12-3.28 (m, 2H), 2.38 (s, |
| | | 3H), 2.05-2.15 (m, 1H), 1.80-1.93 (m, 2H), 1.62-1.71 (m, |
| | | 1H) |
| I-677 | 364.2 | 1H NMR (400 MHz, DMSO-d6) δ = 10.03 (s, 1H), 9.71 (s, |
| | | 1H), 9.41 (d, J = 7.2 Hz, 1H), 8.57 (s, 1H), 8.12 (d, J = 1.6 Hz, |
| | | 1H), 7.85 (dd, J = 1.6, 8.0 Hz, 1H), 7.67 (s, 1H), 7.51 (d, J = 8.0 |
| | | Hz, 1H), 7.12 (dd, J = 1.6, 7.2 Hz, 1H), 4.55 (s, 2H), 3.36 (s, |
| | | 3H), 2.37 (s, 3H) |
| I-676 | 439.2 | 1H NMR (400 MHz, DMSO-d6): δ ppm 10.13 (s, 1H), 9.53 (d, |
| | | J = 7.2 Hz, 1H), 8.67 (s, 1H), 8.10 (d, J = 1.6 Hz, 1H), 7.99 (s, |
| | | 1H), 7.84 (dd, J = 1.6, 8.0 Hz, 1H), 7.51 (d, J = 8.0 Hz, 1H), |
| | | 7.35 (dd, J = 2.0, 7.2 Hz, 1H), 5.11 (t, J = 7.6 Hz, 1H), 3.71- |
| | | 3.59 (m, 1H), 3.41-3.38 (m, 1H), 2.69-2.62 (m, 2H), 2.37 (s, |
| | | 3H), 2.07 (t, J = 18.8 Hz, 3H) |
| I-675 | 368.2 | 1H NMR (400 MHz, DMSO-d6) δ = 10.08 (s, 1H), 9.43 (d, J = |
| | | 7.6 Hz, 1H), 8.60 (s, 1H), 8.06 (d, J = 1.2 Hz, 1H), 7.98 (d, J = |
| | | 2.0 Hz, 1H), 7.80 (dd, J = 1.6, 8.0 Hz, 1H), 7.49 (d, J = 8.0 Hz, |
| | | 1H), 7.26 (dd, J = 2.0, 7.6 Hz, 1H), 2.66 (s, 3H), 2.36 (s, 3H) |
| I-674 | 442.2 | 1H NMR (400 MHz, DMSO-d6): δ ppm 10.13 (s, 1H), 9.53 |
| | | (d, J = 7.2 Hz, 1H), 8.67 (s, 1H), 8.09 (d, J = 1.6 Hz, 1H), 7.99 |
| | | (s, 1H), 7.82 (dd, J = 1.6, 8.0 Hz, 1H), 7.50 (d, J = 8.0 Hz, 1H), |
| | | 7.35 (dd, J = 1.6, 7.2 Hz, 1H), 5.03 (d, J = 5.2 Hz, 1H), 4.23- |
| | | 4.11 (m, 1H), 3.13-2.98 (m, 2H), 2.37 (s, 3H), 2.07 (t, J = 19.2 |
| | | Hz, 3H), 1.21 (d, J = 6.0 Hz, 3H). |
| I-673 | 442.2 | 1H NMR (400 MHz, DMSO-d6) δ = 10.13 (s, 1H), 9.53 (d, J = |
| | | 7.2 Hz, 1H), 8.67 (s, 1H), 8.09 (d, J = 1.6 Hz, 1H), 7.99 (s, 1H), |
| | | 7.82 (dd, J = 1.6, 8.0 Hz, 1H), 7.50 (d, J = 8.0 Hz, 1H), 7.35 |
| | | (dd, J = 1.6, 7.2 Hz, 1H), 5.03 (d, J = 5.2 Hz, 1H), 4.31-4.04 |
| | | (m, 1H), 3.13-2.96 (m, 2H), 2.37 (s, 3H), 2.07 (t, J = 19.2 Hz, |
| | | 3H), 1.21 (d, J = 6.0 Hz, 3H) |
| I-672 | 461.2 | 1H NMR (400 MHz, DMSO-d6) δ = 10.05 (s, 1H), 9.46 (d, J = |
| | | 7.6 Hz, 1H), 8.61 (s, 1H), 8.40 (s, 1H), 8.28 (s, 1H), 8.08 (d, J = |
| | | 1.2 Hz, 1H), 7.82 (dd, J = 1.6, 8.0 Hz, 1H), 7.76 (dd, J = 1.6, |
| | | 7.2 Hz, 1H), 7.49 (d, J = 8.0 Hz, 1H), 6.21 (t, J = 6.0 Hz, 1H), |
| | | 4.84 (d, J = 5.6 Hz, 2H), 3.02 (q, J = 7.6 Hz, 2H), 2.37 (s, 3H), |
| | | 1.35 (t, J = 7.6 Hz, 3H) |
| I-671 | 475.1 | 1H NMR (400 MHz, DMSO-d6) δ = 10.05 (s, 1H), 9.49 (d, J = |
| | | 7.2 Hz, 1H), 9.30 (d, J = 2.0 Hz, 1H), 8.63 (s, 1H), 8.54 (d, J = |
| | | 2.0 Hz, 1H), 8.36 (s, 1H), 8.10 (d, J = 1.6 Hz, 1H), 7.79-7.85 |
| | | (m, 2H), 7.50 (d, J = 8.0 Hz, 1H), 5.00 (d, J = 5.6 Hz, 1H), 3.86- |
| | | 3.96 (m, 1H), 3.12 (dd, J = 4.4, 14.8 Hz, 1H), 2.95-3.04 (m, |
| | | 1H), 2.38 (s, 3H), 1.44-1.60 (m, 2H), 0.92 (t, J = 7.2 Hz, 3H) |
| I-670 | 472.3 | 1H NMR (400 MHz, METHANOL-d4) δ = 9.47 (d, J = 7.2 Hz, |
| | | 1H), 8.46 (s, 1H), 8.12 (s, 1H), 8.04 (s, 1H), 7.95-7.85 (m, 1H), |
| | | 7.68 (d, J = 2.0 Hz, 1H), 7.64-7.59 (m, 1H), 7.50-7.40 (m, |
| | | 1H), 6.84 (d, J = 2.0 Hz, 1H), 4.10-4.03 (m, 1H), 3.98 (s, 3H), |
| | | 3.16-3.02 (m, 2H), 2.41 (s, 3H), 1.70-1.53 (m, 2H), 1.02 (t, |
| | | J = 7.2 Hz, 3H) |
| I-669 | 422.2 | 1H NMR (400 MHz, DMSO-d6) δ = 10.01 (s, 1H), 9.41 (d, J = |
| | | 7.2 Hz, 1H), 8.57 (s, 1H), 8.08 (d, J = 1.2 Hz, 1H), 7.81 (dd, J = |
| | | 1.6, 8.0 Hz, 1H), 7.67 (s, 1H), 7.49 (d, J = 8.0 Hz, 1H), 7.11 (d, |
| | | J = 7.2 Hz, 1H), 5.15-5.00 (m, 1H), 4.54 (s, 2H), 4.17 (d, J = |
| | | 4.4 Hz, 1H), 3.36 (s, 3H), 3.14-2.96 (m, 2H), 2.36 (s, 3H), |
| | | 1.21 (d, J = 6.0 Hz, 3H) |
| I-668 | 407.3 | 1H NMR (400 MHz, DMSO-d6) δ = 10.07 (s, 1H), 9.53-9.45 |
| | | (m, 1H), 8.58 (s, 1H), 8.08 (d, J = 1.2 Hz, 1H), 7.89-7.79 (m, |
| | | 1H), 7.72-7.64 (m, 1H), 7.50 (d, J = 8.0 Hz, 1H), 7.29-7.15 |
| | | (m, 1H), 5.03-4.90 (m, 1H), 4.10-3.96 (m, 1H), 2.72-2.62 |
| | | (m, 1H), 2.43-2.29 (m, 4H), 1.29 (d, J = 6.4 Hz, 3H) |
| I-667 | 472.3 | 1H NMR (400 MHz, DMSO-d6) δ = 9.97 (s, 1H), 9.39 (d, J = |
| | | 7.2 Hz, 1H), 8.55 (s, 1H), 8.09 (d, J = 1.2 Hz, 1H), 8.01 (s, 1H), |
| | | 7.91 (s, 1H), 7.81 (dd, J = 1.6, 8.0 Hz, 1H), 7.75 (s, 1H), 7.57 |
| | | (dd, J = 1.6, 7.2 Hz, 1H), 7.49 (d, J = 8.0 Hz, 1H), 5.12-4.92 |
| | | (m, 1H), 3.97-3.87 (m, 1H), 3.73 (s, 3H), 3.12 (dd, J = 4.4, |
| | | 14.8 Hz, 1H), 3.04-2.94 (m, 1H), 2.37 (s, 3H), 1.62-1.40 (m, |
| | | 2H), 0.92 (t, J = 7.2 Hz, 3H) |
| I-666 | 475.2 | 1H NMR (400 MHz, DMSO-d6) δ: 10.05-9.96 (m, 1H), 9.41 |
| | | (d, J = 6.8 Hz, 1H), 8.57 (s, 1H), 8.29 (s, 1H), 8.07 (d, J = 1.6 |
| | | Hz, 1H), 7.81 (dd, J = 1.6, 7.6 Hz, 1H), 7.67 (s, 1H), 7.49 (d, J = |
| | | 8.0 Hz, 1H), 7.12 (dd, J = 1.6, 7.2 Hz, 1H), 4.55 (s, 2H), 3.37 |
| | | (s, 3H), 3.16 (t, J = 7.2 Hz, 2H), 2.77 (t, J = 7.2 Hz, 2H), 2.39 |
| | | (d, J = 4.4 Hz, 4H), 2.36 (s, 3H), 1.52-1.42 (m, 4H), 1.41- |
| | | 1.32 (m, 2H) |
| I-665 | 436.3 | 1H NMR (400 MHz, DMSO-d6) δ = 10.01 (s, 1H), 9.41 (d, J = |
| | | 7.2 Hz, 1H), 8.57 (s, 1H), 8.08 (d, J = 1.6 Hz, 1H), 7.81 (dd, J = |
| | | 1.6, 8.0 Hz, 1H), 7.67 (s, 1H), 7.49 (d, J = 8.0 Hz, 1H), 7.12 |
| | | (dd, J = 1.6, 7.2 Hz, 1H), 5.00 (d, J = 6.0 Hz, 1H), 4.54 (s, 2H), |
| | | 3.97-3.85 (m, 1H), 3.36 (s, 3H), 3.12 (dd, J = 4.4, 14.8 Hz, |
| | | 1H), 3.03-2.93 (m, 1H), 2.36 (s, 3H), 1.60-1.41 (m, 2H), 0.92 |
| | | (t, J = 7.2 Hz, 3H) |
| I-664 | 432.1 | 1H NMR (400 MHz, DMSO-d6) δ = 10.03 (s, 1H), 9.41 (d, J = |
| | | 7.2 Hz, 1H), 8.58 (s, 1H), 8.17 (d, J = 1.6 Hz, 1H), 8.13 (s, 1H), |
| | | 7.87 (dd, J = 1.6, 8.0 Hz, 1H), 7.67 (s, 1H), 7.56 (d, J = 8.0 Hz, |
| | | 1H), 7.12 (dd, J = 1.6, 7.2 Hz, 1H), 4.55 (s, 2H), 3.37 (s, 3H), |
| | | 2.40 (s, 3H) |
| I-662 | 422.2 | 1H NMR (400 MHz, DMSO-d6): δ ppm 10.00 (s, 1H), 9.41 |
| | | (d, J = 6.8 Hz, 1H), 8.57 (s, 1H), 8.08 (d, J = 1.6 Hz, 1H), 7.81 |
| | | (dd, J = 1.6, 8.0 Hz, 1H), 7.69-7.65 (m, 1H), 7.49 (d, J = 8.0 |
| | | Hz, 1H), 7.12 (dd, J = 1.6, 7.2 Hz, 1H), 5.11-4.96 (m, 1H), |
| | | 4.55 (s, 2H), 4.25-4.08 (m, 1H), 3.36 (s, 3H), 3.12-2.98 (m, |
| | | 2H), 2.36 (s, 3H), 1.21 (d, J = 6.4 Hz, 3H) |
| I-661 | 507.3 | 1H NMR (400 MHz, DMSO-d6) δ = 9.88 (s, 1H), 9.27 (d, J = |
| | | 7.6 Hz, 1H), 8.45 (s, 1H), 8.06 (d, J = 1.6 Hz, 1H), 7.78 (dd, J = |
| | | 1.6, 8.0 Hz, 1H), 7.47 (d, J = 8.0 Hz, 1H), 7.14 (d, J = 2.4 Hz, |
| | | 1H), 6.87 (dd, J = 2.4, 7.6 Hz, 1H), 5.07-4.96 (m, 1H), 4.31 (t, |
| | | J = 8.8 Hz, 1H), 4.15 (t, J = 5.6 Hz, 2H), 4.06 (dd, J = 5.6, 8.4 |
| | | Hz, 1H), 3.91-3.81 (m, 1H), 3.65-3.57 (m, 1H), 3.46-3.48 |
| | | (m, 2H), 3.23-3.27 (m, 1H), 2.66 (s, 3H), 2.35 (s, 3H), 2.07- |
| | | 1.96 (m, 2H) |
| I-660 | 563.3 | 1H NMR (400 MHz, DMSO-d6) δ = 9.88 (s, 1H), 9.27 (d, J = |
| | | 7.6 Hz, 1H), 8.45 (s, 1H), 8.06 (d, J = 1.6 Hz, 1H), 7.78 (dd, J = |
| | | 2.0, 8.0 Hz, 1H), 7.47 (d, J = 8.0 Hz, 1H), 7.15 (d, J = 2.4 Hz, |
| | | 1H), 6.86 (dd, J = 2.8, 7.8 Hz, 1H), 4.89-4.72 (m, 1H), 4.67- |
| | | 4.52 (m, 4H), 4.11 (t, J = 5.8 Hz, 2H), 3.43-3.38 (m, 2H), 2.66 |
| | | (s, 3H), 2.35 (s, 3H), 1.91-1.97 (m, 2H), 1.36 (s, 9H) |
| I-659 | 453.2 | 1H NMR (400 MHz, DMSO-d6): δ ppm 10.13 (s, 1H), 9.53 |
| | | (d, J = 7.2 Hz, 1H), 8.67 (s, 1H), 8.10 (d, J = 1.6 Hz, 1H), 7.99 |
| | | (s, 1H), 7.84 (dd, J = 1.6, 8.0 Hz, 1H), 7.51 (d, J = 8.0 Hz, 1H), |
| | | 7.35 (dd, J = 1.6, 7.2 Hz, 1H), 4.40 (t, J = 8.0 Hz, 1H), 3.43- |
| | | 3.38 (m, 1H), 3.07-2.98 (m, 1H), 2.49-2.39 (m, 2H), 2.39- |
| | | 2.34 (m, 6H), 2.07 (t, J = 19.2 Hz, 3H) |
| I-658 | 382.2 | 1H NMR (400 MHz, DMSO-d6) δ = 10.28 (s, 1H), 9.41 (d, J = |
| | | 7.2 Hz, 1H), 8.61 (s, 1H), 8.40 (dd, J = 2.0, 7.2 Hz, 1H), 7.83- |
| | | 7.91 (m, 1H), 7.68 (s, 1H), 7.53 (dd, J = 8.8, 10.4 Hz, 1H), 7.14 |
| | | (dd, J = 1.6, 7.2 Hz, 1H), 4.55 (s, 2H), 3.37 (s, 3H), 2.68 (s, 3H) |
| I-657 | 418.2 | 1H NMR (400 MHz, DMSO-d6) δ = 10.04 (s, 1H), 9.22 (s, |
| | | 1H), 8.83 (s, 1H), 8.47 (dd, J = 2.0, 7.6 Hz, 1H), 8.27 (d, J = 9.6 |
| | | Hz, 1H), 8.03 (dd, J = 1.6, 9.2 Hz, 1H), 7.82-7.88 (m, 1H), 7.81 |
| | | (d, J = 2.4 Hz, 1H), 7.51 (dd, J = 8.8, 10.4 Hz, 1H), 6.93 (d, J = |
| | | 2.0 Hz, 1H), 3.92 (s, 3H), 2.68 (s, 3H) |
| I-656 | 436.3 | 1H NMR (400 MHz, DMSO-d6) δ = 10.00 (s, 1H), 9.41 (d, J = |
| | | 7.2 Hz, 1H), 8.57 (s, 1H), 8.08 (d, J = 1.6 Hz, 1H), 7.81 (dd, J = |
| | | 1.6, 8.0 Hz, 1H), 7.67 (s, 1H), 7.49 (d, J = 8.0 Hz, 1H), 7.12 (dd, |
| | | J = 1.6, 7.2 Hz, 1H), 5.00 (d, J = 5.6 Hz, 1H), 4.55 (s, 2H), 3.98- |
| | | 3.84 (m, 1H), 3.36 (s, 3H), 3.09-3.14 (m, 1H), 3.03-2.95 (m, |
| | | 1H), 2.36 (s, 3H), 1.59-1.40 (m, 2H), 0.92 (t, J = 7.2 Hz, 3H) |
| I-655 | 419.2 | 1H NMR (400 MHz, DMSO-d6) δ = 10.01 (s, 1H), 9.42 (d, J = |
| | | 7.2 Hz, 1H), 8.57 (s, 1H), 8.10 (d, J = 1.4 Hz, 1H), 7.82 (dd, J = |
| | | 1.6, 8.0 Hz, 1H), 7.67 (s, 1H), 7.50 (s, 1H), 7.14-7.09 (m, 1H), |
| | | 5.11 (t, J = 7.6 Hz, 1H), 4.55 (s, 2H), 3.68-3.61 (m, 1H), 3.38- |
| | | 3.34 (m, 5 H), 2.69-2.62 (m, 2H), 2.37 (s, 3H) |
| I-654 | 483.3 | 1H NMR (400 MHz, DMSO-d6) δ = 9.76 (s, 1H), 9.22 (s, 1H), |
| | | 8.79 (s, 1H), 8.27 (d, J = 9.2 Hz, 1H), 8.12 (d, J = 1.6 Hz, 1H), |
| | | 8.01 (dd, J = 1.6, 9.2 Hz, 1H), 7.82-7.78 (m, 2H), 7.48 (d, J = |
| | | 8.0 Hz, 1H), 6.93 (d, J = 2.4 Hz, 1H), 3.92 (s, 3H), 3.83 (dd, J = |
| | | 6.4, 8.8 Hz, 1H), 3.09-3.03 (m, 1H), 2.45 (d, J = 8.0 Hz, 1H), |
| | | 2.37 (s, 3H), 2.34 (s, 3H), 2.33-2.25 (m, 1H), 2.15-2.07 (m, |
| | | 1H), 1.98-1.85 (m, 2H) |
| I-652 | 470.2 | 1H NMR (400 MHz, DMSO-d6) δ = 9.80-9.69 (m, 1H), 9.21 |
| | | (s, 1H), 8.78 (s, 1H), 8.26 (d, J = 9.2 Hz, 1H), 8.11 (d, J = 1.6 |
| | | Hz, 1H), 8.00 (dd, J = 1.6, 9.2 Hz, 1H), 7.84-7.76 (m, 2H), |
| | | 7.47 (d, J = 8.0 Hz, 1H), 6.92 (d, J = 2.4 Hz, 1H), 5.39 (d, J = |
| | | 7.2 Hz, 1H), 4.22-4.08 (m, 1H), 3.92 (s, 3H), 3.37-3.34 (m, |
| | | 1H), 2.72-2.68 (m, 2H), 2.37 (s, 3H), 2.31-2.17 (m, 2H) |
| I-651 | 419.2 | 1H NMR (400 MHz, DMSO-d6) δ = 10.01 (s, 1H), 9.42 (d, J = |
| | | 6.8 Hz, 1H), 8.58 (s, 1H), 8.11 (d, J = 1.6 Hz, 1H), 7.89-7.77 |
| | | (m, 1H), 7.68 (s, 1H), 7.50 (d, J = 8.0 Hz, 1H), 7.17-7.07 (m, |
| | | 1H), 5.12 (t, J = 7.6 Hz, 1H), 4.55 (s, 2H), 3.70-3.57 (m, 1H), |
| | | 3.41-3.35 (m, 5H), 2.73-2.61 (m, 2H), 2.38 (s, 3H) |
| I-650 | 439.1 | 1H NMR (400 MHz, DMSO-d6) δ = 10.14 (s, 1H), 9.54 (d, J = |
| | | 7.2 Hz, 1H), 8.68 (s, 1H), 8.11 (d, J = 1.6 Hz, 1H), 8.00 (s, 1H), |
| | | 7.87-7.80 (m, 1H), 7.51 (d, J = 8.0 Hz, 1H), 7.39-7.32 (m, |
| | | 1H), 5.14-5.10 (m, 1H), 3.70-3.54 (m, 1H), 3.41-3.35 (m, |
| | | 2H), 2.70-2.62 (m, 2H), 2.38 (s, 3H), 2.08 (t, J = 19.2 Hz, 3H) |
| I-649 | 472.2 | 1H NMR (400 MHz, DMSO-d6) δ = 10.05 (s, 1H), 9.44 (d, J = |
| | | 7.2 Hz, 1H), 8.61 (s, 1H), 8.09 (d, J = 1.2 Hz, 1H), 7.90 (d, J = |
| | | 0.8 Hz, 1H), 7.85-7.80 (m, 2H), 7.50 (d, J = 8.0 Hz, 1H), |
| | | 7.42-7.36 (m, 2H), 5.00 (d, J = 6.0 Hz, 1H), 3.97-3.88 (m, 1H), |
| | | 3.86 (s, 3H), 3.12 (dd, J = 4.4, 14.8 Hz, 1H), 3.03-2.95 (m, |
| | | 1H), 2.37 (s, 3H), 1.60-1.41 (m, 2H), 0.92 (t, J = 7.2 Hz, 3H) |
| I-648 | 466.2 | 1H NMR (400 MHz, DMSO-d6) δ = 9.92 (s, 1H), 9.27 (d, J = |
| | | 7.6 Hz, 1H), 8.46 (s, 1H), 8.06 (d, J = 1.6 Hz, 1H), 7.90-7.78 |
| | | (m, 1H), 7.49 (d, J = 8.0 Hz, 1H), 7.17 (d, J = 2.4 Hz, 1H), |
| | | 6.94-6.79 (m, 1H), 4.63-4.57 (m, 1H), 4.51-4.45 (m, 1H), 4.35 (t, |
| | | J = 6.8 Hz, 1H), 3.90 (s, 3H), 3.82 (t, J = 4.8 Hz, 1H), 3.66- |
| | | 3.62 (m, 1H), 3.59-3.54 (m, 1H), 2.70-2.61 (m, 2H), 2.54- |
| | | 2.47 (m, 3H), 2.36 (s, 3H) |
| I-647 | 470.2 | 1H NMR (400 MHz, DMSO-d6) δ = 10.12 (s, 1H), 9.44 (d, J = |
| | | 7.6 Hz, 1H), 8.61 (s, 1H), 8.06 (s, 1H), 7.99 (d, J = 2.0 Hz, 1H), |
| | | 7.91-7.78 (m, 1H), 7.51 (d, J = 8.0 Hz, 1H), 7.34-7.16 (m, |
| | | 1H), 4.64-4.55 (m, 1H), 4.50-4.42 (m, 1H), 4.35 (t, J = 6.8 |
| | | Hz, 1H), 3.83 (t, J = 4.8 Hz, 1H), 3.67-3.60 (m, 1H), 3.59- |
| | | 3.53 (m, 1H), 2.71-2.61 (m, 2H), 2.54-2.47 (m, 2H), 2.37 (s, |
| | | 3H) |
| I-646 | 466.3 | 1H NMR (400 MHz, DMSO-d6) δ = 9.90 (s, 1H), 9.28 (d, J = |
| | | 7.6 Hz, 1H), 8.46 (s, 1H), 8.07 (d, J = 1.6 Hz, 1H), 7.85-7.76 |
| | | (m, 1H), 7.48 (d, J = 8.0 Hz, 1H), 7.17 (d, J = 2.4 Hz, 1H), |
| | | 6.93-6.83 (m, 1H), 4.65-4.55 (m, 1H), 4.51-4.43 (m, 1H), 4.10 (t, |
| | | J = 7.2 Hz, 1H), 3.90 (s, 3H), 3.68-3.60 (m, 1H), 3.60-3.54 |
| | | (m, 1H), 3.53-3.42 (m, 1H), 2.85-2.73 (m, 2H), 2.36 (s, 3H), |
| | | 2.33-2.20 (m, 2H) |
| I-643 | 470.1 | 1H NMR (400 MHz, DMSO-d6) δ = 10.10 (s, 1H), 9.44 (d, J = |
| | | 7.6 Hz, 1H), 8.61 (s, 1H), 8.08 (d, J = 1.6 Hz, 1H), 7.99 (d, J = |
| | | 2.0 Hz, 1H), 7.89-7.78 (m, 1H), 7.50 (d, J = 8.0 Hz, 1H), 7.31- |
| | | 7.22 (m, 1H), 4.63-4.54 (m, 1H), 4.51-4.42 (m, 1H), 4.11 (t, |
| | | J = 7.2 Hz, 1H), 3.68-3.62 (m, 1H), 3.59-3.55 (m, 1H), 3.53- |
| | | 3.43 (m, 1H), 2.84-2.72 (m, 2H), 2.37 (s, 3H), 2.34-2.22 (m, |
| | | 2H) |

### Example 96—HTRF Biochemical Assay for wtKIT, PDGFRα and CSF-1R

Exemplary compounds were tested for ability to inhibit the phosphorylation of a peptide substrate by the tyrosine kinase wt KIT, PDGFRα, or CSF-1R. Assay procedures and results are described below.

### Part I—Procedures for HTRF Assay

Enzyme, substrate, and cofactors (ATP and Mn²⁺) are combined in a well of a microtiter plate and incubated for 3 hours at 25° C. At the end of the incubation, the reaction is quenched by the addition of an EDTA-containing buffer.

### Assay Parameters:

### Peptide Substrate

The substrate used in the CSF-1R and PDGFRα assay is FAM-KKKKEEIYFFF-CONH₂ (FAM is carboxyfluorescein). Peptide should be >95% purity. The substrate for the KIT assay is FAM-GEEPLYWSFPAKKK-NH₂.

### Assay Setup & Conditions

1. To a well of a 384-wellplate add 5 μL of 2× enzyme buffer (or control).
2. Add 100 nL of 100× compound. Enzyme and compound may be pre-incubated at this time if desired.
3. Add 5 μL of 2× substrate buffer.
4. Incubate plate at 25° C. for 3 hours.
5. Add 10 uL of anti-phosphotyrosine antibody buffer.
6. Read plate in BioTek Synergy Reader.
   Reaction Conditions for wtKIT Assay

### Final Assay Reaction Mixture

100 mM HEPES, pH 7.5 0.1% BSA
0.01% Triton x-100
1 mM DTT
10 mM MnCl₂
10 μM Sodium Orthovanadate
10 μM Beta-Glycerophosphate
400 μM ATP
1% DMSO (from compound)
1 μM FAM-KKKKEEIYFFF-CONH₂, 5.0 nM wtKIT Enzyme (1888289AM)*
*Specific activity may vary from lot to lot. Enzyme concentration may need to be adjusted to yield 10-20% conversion of substrate to product.

TABLE 3
Protein Lots and assay conditions

| | | | Vendor and | | ATP | Substrate | Incubation |
|---|---|---|---|---|---|---|---|
| Protein | | Assay | catalog | [Enzyme], | Conc | Conc | Time |
| lot | Assay | Platform | number | nM | (μM) | (μM) | (h) |
| 1 | CSF-1R | Caliper | Invitrogen.PV | 0.25 | 100 | 1 | 3 |
| | | MSA | 3249.662393 | | | | |
| | | | N | | | | |
| 2 | KIT | Caliper | Invitrogen.P3 | 4 | 400 | 1 | 17 |
| | | MSA | 081.1888289 | | | | |
| | | | AF | | | | |
| 3 | PDGF | Caliper | Millipore.14- | 0.5 | 35 | 1 | 3 |
| | R-a | MSA | 467.26321_A | | | | |
| | | | CTI | | | | |

### Part II—Results

Experimental results are provided in Table 4, below. The symbol “****” indicates an IC₅₀ less than or equal to 0.05 μM. The symbol “***” indicates an IC₅₀ in the range of greater than 0.05 μM and less than or equal to 0.5 μM. The symbol “**” indicates an IC₅₀ in the range of greater than 0.5 μM and less than or equal to 5 μM. The symbol “*” indicates an IC₅₀ in the range of greater than 5 μM to 30 μM.

TABLE 4

| | KIT | CSF-1R | PDFGRα |
|---|---|---|---|
| | Biochemical | Biochemical | Biochemical |
| Compound | IC₅₀ avg | IC₅₀ avg | IC₅₀ avg |
| No. | (μM) | (μM) | (μM) |
| I-71 | **** | **** | * |
| I-72 | **** | **** | ** |
| I-73 | **** | **** | *** |
| I-75 | **** | *** | ** |
| I-76 | **** | **** | *** |
| I-77 | **** | *** | * |
| I-78 | **** | *** | ** |
| I-79 | **** | *** | *** |
| I-80 | *** | *** | * |
| I-81 | *** | ** | * |
| I-84 | **** | **** | ** |
| I-85 | **** | **** | ** |
| I-86 | **** | **** | *** |
| I-87 | *** | *** | * |
| I-88 | **** | **** | ** |
| I-89 | **** | *** | ** |
| I-90 | **** | **** | *** |
| I-91 | **** | *** | *** |
| I-92 | **** | **** | *** |
| I-93 | **** | **** | *** |
| I-94 | **** | **** | *** |
| I-95 | **** | *** | ** |
| I-96 | **** | **** | *** |
| I-97 | **** | *** | ** |
| I-98 | **** | *** | ** |
| I-99 | *** | *** | *** |
| I-100 | **** | *** | *** |
| I-101 | **** | *** | * |
| I-102 | **** | **** | ** |
| I-103 | **** | **** | ** |
| I-104 | **** | *** | *** |
| I-105 | **** | **** | ** |
| I-106 | **** | **** | *** |
| I-107 | **** | **** | *** |
| I-108 | **** | **** | *** |
| I-109 | **** | *** | ** |
| I-110 | **** | ** | ** |
| I-111 | **** | *** | *** |
| I-112 | **** | **** | ** |
| I-113 | **** | *** | *** |
| I-122 | **** | *** | ** |
| I-114 | **** | *** | * |
| I-115 | **** | *** | ** |
| I-116 | *** | ** | * |
| I-117 | **** | *** | * |
| I-119 | **** | ** | ** |
| I-121 | **** | **** | *** |
| I-120 | *** | * | * |
| I-132 | **** | * | ** |
| I-142 | **** | *** | ** |
| I-149 | **** | ** | ** |
| I-154 | **** | *** | ** |
| I-157 | *** | ** | * |
| I-173 | **** | **** | *** |
| I-189 | **** | ** | ** |
| I-205 | **** | | |
| I-206 | **** | | |
| I-207 | **** | | |
| I-208 | *** | | |
| I-209 | *** | | |
| I-210 | *** | | |
| I-211 | *** | | |
| I-213 | *** | | |
| I-214 | *** | | |
| I-215 | *** | | |
| I-216 | **** | | |
| I-217 | **** | | |
| I-218 | **** | | |
| I-219 | **** | | |
| I-222 | *** | | |
| I-223 | *** | | |
| I-224 | *** | | |
| I-226 | *** | | |
| I-227 | *** | | |
| I-228 | *** | | |
| I-229 | **** | | |
| I-230 | **** | | |
| I-232 | *** | | |
| I-233 | *** | | |
| I-234 | *** | | |
| I-235 | *** | | |
| I-236 | *** | | |
| I-237 | *** | | |
| I-238 | *** | | |
| I-239 | *** | | |
| I-242 | **** | | |
| I-243 | **** | *** | *** |
| I-244 | *** | | |
| I-245 | **** | | |
| I-246 | *** | | |
| I-247 | *** | | |
| I-248 | *** | | |
| I-249 | *** | | |
| I-250 | *** | | |
| I-256 | *** | | |
| I-257 | **** | | |
| I-258 | *** | | |
| I-259 | **** | | |
| I-260 | *** | | |
| I-261 | **** | | |
| I-262 | **** | | |
| I-263 | **** | | |
| I-264 | **** | | |
| I-265 | *** | | |
| I-266 | **** | | |
| I-288 | **** | *** | ** |
| I-296 | **** | **** | ** |
| I-306 | *** | ** | ** |
| I-335 | *** | | |
| I-336 | *** | *** | *** |
| I-359 | *** | | |
| I-360 | **** | | |
| I-369 | **** | * | * |
| I-370 | *** | | |
| I-373 | **** | | |
| I-394 | *** | | |
| I-396 | **** | *** | ** |
| I-118 | *** | ** | * |
| I-129 | **** | **** | ** |
| I-212 | *** | | |
| I-220 | **** | | |
| I-221 | *** | | |
| I-225 | **** | ** | * |
| I-240 | *** | | |
| I-339 | **** | ** | ** |
| I-450 | **** | | |
| I-451 | **** | | |
| I-452 | **** | | |
| I-453 | **** | | |
| I-454 | **** | | |
| I-455 | **** | | |
| I-456 | **** | | |
| I-457 | **** | | |
| I-458 | *** | | |
| I-459 | *** | | |
| I-460 | *** | | |
| I-461 | **** | | |
| I-462 | *** | | |
| I-463 | *** | | |
| I-464 | *** | | |
| I-465 | *** | | |
| I-466 | **** | | |
| I-467 | **** | | |
| I-468 | **** | | |
| I-469 | **** | | |
| I-470 | **** | | |
| I-471 | **** | | |
| I-472 | *** | | |
| I-473 | *** | | |
| I-474 | **** | | |
| I-475 | **** | | |
| I-476 | **** | | |
| I-477 | **** | | |
| I-478 | **** | | |
| I-479 | **** | | |
| I-480 | **** | | |
| I-481 | **** | | |
| I-482 | **** | | |
| I-483 | **** | | |
| I-484 | *** | | |
| I-485 | *** | | |
| I-486 | **** | | |
| I-487 | *** | | |
| I-490 | **** | | |
| I-491 | *** | | |
| I-492 | *** | | |
| I-493 | **** | | |
| I-494 | **** | | |
| I-495 | **** | | |
| I-496 | **** | | |
| I-497 | *** | | |
| I-498 | **** | | |
| I-499 | **** | | |
| I-500 | **** | | |
| I-501 | **** | | |
| I-502 | ** | | |
| I-503 | **** | | |
| I-506 | **** | | |
| I-507 | *** | | |
| I-508 | **** | | |
| I-509 | **** | | |
| I-510 | *** | | |
| I-511 | *** | | |
| I-512 | *** | | |
| I-513 | *** | | |
| I-514 | *** | | |
| I-515 | **** | | |
| I-516 | **** | | |
| I-517 | **** | | |
| I-518 | **** | | |
| I-519 | **** | | |
| I-520 | *** | | |
| I-640 | *** | | |
| I-521 | **** | | |
| I-522 | *** | | |
| I-641 | ** | | |
| I-523 | **** | | |
| I-524 | *** | | |
| I-525 | **** | | |
| I-526 | **** | | |
| I-527 | *** | | |
| I-528 | *** | | |
| I-529 | **** | | |
| I-530 | **** | | |
| I-531 | **** | | |
| I-532 | **** | | |
| I-533 | **** | | |
| I-534 | *** | | |
| I-535 | **** | | |
| I-536 | **** | | |
| I-537 | **** | | |
| I-538 | *** | | |
| I-539 | **** | | |
| I-540 | **** | | |
| I-541 | **** | | |
| I-542 | **** | | |
| I-543 | *** | | |
| I-544 | *** | | |
| I-545 | *** | | |
| I-546 | *** | | |
| I-547 | **** | | |
| I-548 | **** | | |
| I-549 | **** | | |
| I-550 | *** | | |
| I-551 | **** | | |
| I-552 | **** | | |
| I-553 | **** | | |
| I-554 | **** | | |
| I-555 | **** | | |
| I-556 | **** | | |
| I-557 | * | | |
| I-558 | ** | | |
| I-559 | **** | | |
| I-560 | **** | | |
| I-561 | **** | | |
| I-562 | **** | | |
| I-563 | *** | | |
| I-564 | *** | | |
| I-565 | *** | | |
| I-566 | **** | | |
| I-567 | *** | | |
| I-568 | **** | | |
| I-569 | *** | | |
| I-570 | **** | | |
| I-571 | **** | | |
| I-572 | *** | | |
| I-573 | *** | | |
| I-574 | **** | | |
| I-575 | **** | | |
| I-576 | **** | | |
| I-577 | **** | | |
| I-578 | *** | | |
| I-579 | *** | | |
| I-580 | **** | | |
| I-581 | *** | | |
| I-582 | **** | | |
| I-583 | **** | | |
| I-584 | ** | | |
| I-585 | **** | | |
| I-586 | **** | | |
| I-587 | **** | | |
| I-588 | *** | | |
| I-589 | ** | | |
| I-590 | **** | | |
| I-591 | *** | | |
| I-592 | **** | | |
| I-593 | *** | | |
| I-594 | **** | | |
| I-595 | **** | | |
| I-597 | **** | | |
| I-598 | *** | | |
| I-599 | *** | | |
| I-600 | *** | | |
| I-601 | **** | | |
| I-602 | **** | | |
| I-603 | **** | | |
| I-604 | ** | | |
| I-605 | *** | | |
| I-606 | *** | | |
| I-607 | ** | | |
| I-608 | *** | | |
| I-609 | **** | | |
| I-610 | *** | | |
| I-611 | **** | | |
| I-612 | *** | | |
| I-613 | **** | | |
| I-614 | ** | | |
| I-615 | **** | | |
| I-616 | **** | | |
| I-617 | ** | | |
| I-618 | **** | | |
| I-619 | *** | | |
| I-620 | *** | | |
| I-621 | **** | | |
| I-622 | **** | | |
| I-623 | ** | | |
| I-624 | **** | | |
| I-625 | **** | | |
| I-626 | *** | | |
| I-627 | **** | | |
| I-628 | **** | | |
| I-629 | **** | | |
| I-630 | **** | | |
| I-631 | **** | | |
| I-632 | *** | | |
| I-633 | **** | | |
| I-752 | **** | | |
| I-753 | **** | | |
| I-665 | **** | | |
| I-666 | *** | | |
| I-667 | **** | | |
| I-668 | *** | | |
| I-669 | **** | | |
| I-670 | **** | | |
| I-671 | **** | | |
| I-672 | **** | | |
| I-673 | *** | | |
| I-674 | *** | | |
| I-675 | **** | | |
| I-676 | **** | | |
| I-677 | *** | | |
| I-678 | **** | | |
| I-679 | **** | | |
| I-680 | **** | | |
| I-681 | **** | | |
| I-682 | *** | | |
| I-683 | *** | | |
| I-684 | **** | | |
| I-685 | **** | | |
| I-686 | **** | | |
| I-687 | *** | | |
| I-688 | **** | | |
| I-689 | **** | | |
| I-690 | *** | | |
| I-691 | **** | | |
| I-692 | **** | | |
| I-693 | **** | | |
| I-694 | **** | | |
| I-695 | **** | | |
| I-696 | **** | | |
| I-697 | *** | | |
| I-698 | **** | | |
| I-699 | *** | | |
| I-700 | *** | | |
| I-703 | **** | | |
| I-706 | **** | | |
| I-707 | **** | | |
| I-708 | *** | | |
| I-709 | **** | | |
| I-710 | **** | | |
| I-711 | *** | | |
| I-712 | *** | | |
| I-713 | **** | | |
| I-714 | **** | | |
| I-715 | **** | | |
| I-716 | **** | | |
| I-717 | **** | | |
| I-718 | **** | | |
| I-719 | **** | | |
| I-720 | **** | | |
| I-721 | **** | | |
| I-722 | **** | | |
| I-723 | **** | | |
| I-724 | **** | | |
| I-725 | **** | | |
| I-726 | *** | | |
| I-727 | **** | | |
| I-729 | **** | | |
| I-730 | **** | | |
| I-731 | *** | | |
| I-732 | *** | | |
| I-733 | **** | | |
| I-734 | **** | | |
| I-735 | ** | | |
| I-736 | **** | | |
| I-737 | **** | | |
| I-738 | *** | | |
| I-739 | **** | | |
| I-741 | ** | | |
| I-742 | *** | | |
| I-743 | ** | | |
| I-744 | ** | | |
| I-745 | **** | | |
| I-746 | ** | | |
| I-747 | *** | | |
| I-748 | *** | | |
| I-749 | *** | | |
| I-750 | *** | | |
| I-754 | **** | | |
| I-447 | *** | | |
| I-448 | ** | | |
| I-449 | *** | | |
| I-634 | *** | | |
| I-635 | **** | | |
| I-636 | *** | | |
| I-637 | *** | | |
| I-638 | **** | | |
| I-639 | **** | | |
| I-643 | **** | *** | * |
| I-646 | **** | **** | ** |
| I-647 | **** | *** | * |
| I-648 | **** | **** | ** |
| I-649 | **** | **** | *** |
| I-650 | *** | *** | * |
| I-651 | **** | *** | * |
| I-652 | **** | **** | *** |
| I-654 | **** | **** | *** |
| I-655 | **** | *** | * |
| I-656 | **** | *** | * |
| I-657 | **** | *** | ** |
| I-658 | ** | ** | * |
| I-659 | *** | ** | * |
| I-660 | **** | *** | ** |
| I-661 | **** | *** | ** |
| I-662 | *** | *** | * |
| I-664 | *** | *** | ** |

### Example 97—Caliper Biochemical Assay for wtKIT, PDGFRα and CSF-1R

Part I—Procedures for Biochemical caliper wtKIT Assay

Enzyme, substrate, and cofactors (ATP and Mn²⁺) are combined in a well of a microtiter plate and incubated for 3 hours at 25° C. At the end of the incubation, the reaction is quenched by the addition of an EDTA-containing buffer. Substrate and product are separated electrophoretically using the microfluidic-based LabChip 3000 Drug Discovery System from Caliper Life Sciences* and quantitated by fluorescence intensity.

### Assay Parameters:

### Peptide Substrate

The substrate used in the CSF-1R and PDGFRα assay is FAM-KKKKEEIYFFF-CONH₂ (FAM is carboxyfluorescein). Peptide should be >95% purity. The substrate for the KIT assay is FAM-GEEPLYWSFPAKKK-NH₂.

### Assay Setup & Conditions

1. To a well of a 384-well plate add 5 μL of 2× enzyme buffer (or control)
2. Add 100 nL of 100× compound.
3. Add 5 μL of 2× substrate buffer.
4. Incubate plate at 25° C. for 3 hours.
5. Add 10 uL of anti-phosphotyrosine antibody buffer.
6. Read plate LabChip 3000 Drug Discovery System

### Reaction Conditions

3 hours at 25° C. where 100% Inhibitor: No enzyme.

### Final Assay Reaction Mixture

100 mM HEPES, pH 7.5 0.1% BSA
0.01% Triton X-100
1 mM DTT
10 mM MnCl₂
10 μM Sodium Orthovanadate
10 μM Beta-Glycerophosphate
400 μM ATP
1% DMSO (from compound)
1 μM FAM-KKKKEEIYFFF-CONH₂ 5.0 nM wtKIT Enzyme (1888289AM)*
*Specific activity may vary from lot to lot. Enzyme concentration may need to be adjusted to yield 10-20% conversion of substrate to product.

TABLE 5
Materials & Buffers

ITEM VENDOR PART NUMBER
Enzyme
c-KIT Invitrogen P3081 (lot # 1888289AM)
Substrate
FAM-KKKKEEIYFFF-CONH2 SynPep Custom synthesis
Antibody
Monoclonal Anti-Phosphotyrosine-Biotin antibody
Sigma B1531
Streptavidin-Tb cryptate ScisBio 610SATLA
Control Inhibitor
Staurosporine Biomol EI-156
Buffer Components
HEPES, free acid Calbiochem 391338
HEPES, sodium salt Calbiochem 391333
Triton X-100 Sigma T8787
BSA Sigma A3059
Manganese Chloride Teknova M0350
ATP disodium salt Sigma A7699
DTT (Cleland's Reagent) Calbiochem 233153
Sodium Orthovanadate Sigma S6508
Beta-Glycerophosphate Calbiochem 35675
EDTA, disodium salt, dihydrate VWR VW1474-01
DMSO VWR BJ081-4
Coating Reagent 3 Caliper Life Sciences 760050
Sodium Hydroxide, 50% VWR VW3246-1
Hydrochloric Acid, concentrated JT Baker 9530-33
Sodium Carbonate Mallinckrodt 7521
Sodium Bicarbonate Sigma S-6297

Materials:
1× Core Buffer
100 mM HEPES, pH 7.5 0.1% BSA
0.01% Triton X-100
10 mM MnCl2
1 mM DTT
10 μM Sodium Orthovanadate 10 μM Beta-Glycerophosphate
2× Enzyme Buffer
1× Core Buffer
10 nM c-KIT Enzyme (lot 1888289AM)
2× Substrate Buffer
1× Core Buffer
800 μM ATP
2 μM FAM-KKKKEEIYFFF-CONH₂
2× Anti-phosphotyrosine antibody Buffer
50 mM HEPES
0.05% Brij-35
145 ng/mL Anti-Phosphotyrosine-Biotin antibody 20 ng/ML Streptavidin

### Part II—Results

Experimental results are provided in Table 6, below. The symbol “††††” indicates an IC₅₀ less than or equal to 0.05 μM. The symbol “†††” indicates an IC₅₀ in the range of greater than 0.05 μM and less than or equal to 0.5 μM. The symbol “††” indicates an IC₅₀ in the range of greater than 0.5 μM and less than or equal to 5 μM. The symbol “†” indicates an IC₅₀ in the range of greater than 5 μM to 30 μM.

TABLE 6

| | KIT | CSF-1R | PDGFRα |
|---|---|---|---|
| Compound | Biochemical | Biochemical | Biochemical Avg |
| No. | IC₅₀ avg (μM) | IC₅₀ (μM) | IC₅₀ (μM) |
| I-71 | †††† | †††† | †† |
| I-88 | †††† | †††† | ††† |
| I-114 | †††† | ††† | †† |
| I-123 | †††† | †††† | †† |
| I-124 | †††† | †††† | ††† |
| I-125 | †††† | †††† | ††† |
| I-126 | †††† | ††† | †† |
| I-127 | †††† | †††† | ††† |
| I-128 | †††† | ††† | † |
| I-130 | †††† | †††† | †††† |
| I-131 | †††† | †††† | ††† |
| I-132 | †††† | †† | †† |
| I-133 | †††† | †† | † |
| I-134 | †††† | †† | †† |
| I-135 | †††† | ††† | † |
| I-136 | †††† | †††† | ††† |
| I-137 | ††† | †† | †† |
| I-138 | †††† | †††† | †† |
| I-139 | †††† | ††† | ††† |
| I-140 | †††† | ††† | † |
| I-141 | †††† | †† | †† |
| I-142 | †††† | ††† | †† |
| I-143 | †††† | †††† | ††† |
| I-144 | †††† | ††† | †† |
| I-145 | †††† | ††† | †† |
| I-146 | †††† | ††† | †† |
| I-147 | †††† | ††† | † |
| I-148 | †††† | ††† | †† |
| I-149 | †††† | †††† | † |
| I-150 | †††† | †††† | ††† |
| I-151 | †††† | ††† | †† |
| I-152 | †††† | ††† | †† |
| I-153 | †††† | †††† | ††† |
| I-154 | †††† | †††† | †† |
| I-155 | †††† | †† | † |
| I-156 | †††† | ††† | †† |
| I-157 | †††† | ††† | †† |
| I-158 | †††† | ††† | †† |
| I-159 | ††† | ††† | † |
| I-165 | †††† | †††† | ††† |
| I-166 | †††† | †††† | ††† |
| I-428 | †††† | †† | †† |
| I-167 | †††† | ††† | †† |
| I-168 | †††† | ††† | †† |
| I-170 | †† | †††† | †† |
| I-171 | †††† | †† | †† |
| I-173 | †††† | †††† | ††† |
| I-174 | ††† | †††† | ††† |
| I-176 | †††† | †††† | ††† |
| I-177 | †††† | ††† | ††† |
| I-178 | †††† | †††† | ††† |
| I-179 | ††† | †† | † |
| I-180 | †††† | ††† | †† |
| I-429 | †††† | ††† | † |
| I-181 | †††† | †††† | †† |
| I-182 | †††† | ††† | † |
| I-183 | ††† | ††† | † |
| I-184 | †††† | ††† | †† |
| I-185 | †††† | †††† | ††† |
| I-186 | ††† | ††† | †† |
| I-187 | ††† | ††† | ††† |
| I-188 | †††† | ††† | ††† |
| I-189 | ††† | ††† | †† |
| I-190 | †††† | †††† | †† |
| I-191 | †††† | †††† | ††† |
| I-430 | †††† | †††† | ††† |
| I-193 | †††† | †††† | ††† |
| I-194 | †††† | †††† | ††† |
| I-195 | †††† | †††† | ††† |
| I-196 | †††† | †††† | ††† |
| I-197 | †††† | ††† | †† |
| I-198 | †††† | ††† | †† |
| I-199 | †††† | †††† | ††† |
| I-200 | †††† | †††† | †††† |
| I-201 | †††† | †††† | †††† |
| I-202 | †††† | †††† | ††† |
| I-203 | †††† | ††† | †† |
| I-204 | †††† | ††† | † |
| I-205 | †††† | †††† | ††† |
| I-206 | †††† | | |
| I-207 | †††† | | |
| I-208 | †††† | ††† | ††† |
| I-209 | †††† | ††† | †† |
| I-210 | †††† | | |
| I-211 | †††† | | |
| I-213 | †††† | | |
| I-214 | †††† | | |
| I-215 | †††† | †††† | †† |
| I-216 | †††† | †††† | ††† |
| I-217 | †††† | †††† | ††† |
| I-218 | †††† | | |
| I-219 | †††† | ††† | †† |
| I-222 | ††† | | |
| I-223 | †††† | | |
| I-224 | †††† | | |
| I-226 | ††† | | |
| I-227 | †††† | | |
| I-228 | †††† | †††† | ††† |
| I-229 | †††† | | |
| I-230 | †††† | †††† | ††† |
| I-232 | †††† | †††† | ††† |
| I-233 | ††† | | |
| I-234 | †††† | †††† | ††† |
| I-235 | †††† | ††† | † |
| I-236 | †††† | †† | † |
| I-237 | ††† | †† | † |
| I-238 | †††† | ††† | † |
| I-239 | ††† | | |
| I-242 | †††† | | |
| I-243 | †††† | ††† | ††† |
| I-244 | ††† | | |
| I-245 | †††† | †††† | †††† |
| I-246 | †††† | | |
| I-247 | †††† | | |
| I-248 | ††† | | |
| I-249 | †††† | | |
| I-250 | †††† | | |
| I-251 | †††† | ††† | † |
| I-252 | ††† | † | † |
| I-253 | ††† | †† | †† |
| I-255 | †††† | †††† | †† |
| I-256 | †††† | ††† | † |
| I-257 | †††† | ††† | †† |
| I-258 | †††† | ††† | ††† |
| I-259 | †††† | †††† | ††† |
| I-260 | †††† | | |
| I-261 | †††† | †††† | ††† |
| I-262 | †††† | †††† | ††† |
| I-263 | †††† | †††† | ††† |
| I-264 | †††† | †††† | ††† |
| I-265 | ††† | † | † |
| I-266 | †††† | †††† | ††† |
| I-267 | †††† | †††† | †††† |
| I-268 | ††† | | |
| I-270 | ††† | †††† | † |
| I-271 | †††† | ††† | †† |
| I-272 | †††† | ††† | †† |
| I-273 | †††† | †††† | †† |
| I-274 | †††† | | |
| I-275 | †††† | | |
| I-276 | †††† | | |
| I-277 | †††† | | |
| I-278 | †††† | | |
| I-279 | †††† | †††† | †††† |
| I-280 | †††† | †††† | † |
| I-282 | †††† | † | † |
| I-283 | †††† | †††† | † |
| I-284 | †††† | | |
| I-285 | †††† | | |
| I-286 | †††† | | |
| I-287 | ††† | † | † |
| I-288 | †††† | ††† | †† |
| I-289 | ††† | | |
| I-290 | †††† | | |
| I-291 | †††† | †††† | ††† |
| I-292 | †††† | | |
| I-293 | ††† | | |
| I-294 | †††† | | |
| I-295 | †††† | †††† | †††† |
| I-296 | †††† | †††† | ††† |
| I-297 | †††† | | |
| I-298 | †††† | †††† | †††† |
| I-299 | †††† | †††† | ††† |
| I-300 | †††† | †††† | †††† |
| I-301 | †††† | †††† | †††† |
| I-302 | †††† | †††† | †††† |
| I-303 | †††† | | |
| I-304 | †††† | †††† | †††† |
| I-305 | †††† | †††† | ††† |
| I-306 | †††† | ††† | † |
| I-307 | †††† | †††† | †† |
| I-308 | †††† | | |
| I-309 | †††† | ††† | †† |
| I-310 | ††† | | |
| I-311 | ††† | †††† | †††† |
| I-312 | †††† | †††† | †† |
| I-313 | †††† | | |
| I-314 | †††† | ††† | †† |
| I-315 | †††† | | |
| I-316 | †††† | | |
| I-317 | †††† | | |
| I-318 | †††† | †††† | ††† |
| I-319 | †††† | †††† | ††† |
| I-320 | †††† | †††† | ††† |
| I-321 | †††† | ††† | †† |
| I-322 | †††† | ††† | † |
| I-323 | †††† | †††† | ††† |
| I-324 | †††† | †††† | ††† |
| I-325 | †††† | †††† | ††† |
| I-326 | ††† | †† | † |
| I-327 | ††† | †††† | † |
| I-328 | †††† | ††† | †† |
| I-329 | †††† | †††† | †††† |
| I-330 | †††† | †††† | ††† |
| I-331 | †††† | †††† | ††† |
| I-334 | †††† | | |
| I-335 | ††† | †† | †† |
| I-336 | ††† | | |
| I-337 | †††† | ††† | ††† |
| I-342 | †††† | | |
| I-343 | †††† | | |
| I-344 | †††† | †††† | †† |
| I-345 | ††† | | |
| I-346 | †††† | | |
| I-347 | †††† | ††† | †† |
| I-348 | †††† | ††† | †† |
| I-349 | †††† | | |
| I-350 | †††† | | |
| I-351 | †††† | | |
| I-352 | †††† | | |
| I-353 | †††† | | |
| I-354 | †††† | | |
| I-355 | †††† | | |
| I-356 | †††† | ††† | †† |
| I-357 | ††† | | |
| I-358 | †††† | | |
| I-359 | †††† | ††† | †† |
| I-360 | †††† | †† | †† |
| I-361 | †††† | †††† | ††† |
| I-362 | †††† | ††† | †† |
| I-363 | †††† | ††† | †† |
| I-364 | ††† | †††† | † |
| I-365 | †††† | †††† | †††† |
| I-366 | †††† | | |
| I-367 | †††† | †††† | †††† |
| I-368 | †††† | ††† | ††† |
| I-369 | †††† | ††† | †† |
| I-370 | †††† | †††† | †† |
| I-371 | †††† | | |
| I-372 | ††† | | |
| I-373 | †††† | | |
| I-374 | †††† | | |
| I-375 | †††† | | |
| I-376 | †††† | †††† | ††† |
| I-377 | †††† | †††† | †† |
| I-378 | †††† | | |
| I-379 | ††† | | |
| I-380 | †††† | | |
| I-381 | †††† | †††† | ††† |
| I-382 | †††† | †††† | ††† |
| I-383 | †††† | †††† | ††† |
| I-384 | †††† | †††† | † |
| I-385 | †††† | | |
| I-386 | †††† | | |
| I-387 | †††† | †††† | ††† |
| I-388 | †††† | † | †† |
| I-389 | ††† | | |
| I-390 | ††† | | |
| I-391 | †††† | | |
| I-392 | †††† | †††† | †† |
| I-393 | †††† | †††† | †† |
| I-394 | †††† | ††† | † |
| I-395 | ††† | | |
| I-396 | †††† | ††† | †† |
| I-397 | †††† | | |
| I-398 | ††† | | |
| I-399 | ††† | | |
| I-129 | †††† | †††† | †† |
| I-160 | †††† | †††† | †† |
| I-161 | †††† | †††† | ††† |
| I-162 | †††† | ††† | †† |
| I-163 | †††† | †††† | ††† |
| I-164 | †††† | † | † |
| I-172 | †††† | ††† | †† |
| I-175 | †††† | †††† | † |
| I-192 | †††† | ††† | †† |
| I-212 | ††† | | |
| I-220 | †††† | ††† | † |
| I-221 | †††† | ††† | † |
| I-225 | †††† | ††† | † |
| I-240 | †††† | † | † |
| I-254 | ††† | †† | † |
| I-269 | †††† | ††† | † |
| I-281 | †††† | †† | † |
| I-332 | ††† | | |
| I-333 | †††† | | |
| I-338 | †††† | | |
| I-339 | †††† | †††† | †† |
| I-340 | †††† | †††† | ††† |
| I-341 | †††† | †††† | ††† |

### Example 98—Cell-Based Assay for Inhibiting wtKIT

Exemplary compounds were tested for ability to inhibit KIT phosphorylation using M-07e cells as monitored by pKIT ELISA. M-07e cells are also represented as M-07E, M-07e, M07-e, M07e, Mo7e, MO7e, M07E and MO7E. Assay procedures and results are described below.

Part I—Procedures for Determining KIT Inhibition inM-07e Cells Using pKTT ELISA
Reagents and consumables:
M-07e cells: Initially obtained from Accegen, cat #ABC-TC1313
p-cKIT Capture ELISA antibody: anti-Human Phospho-CD117/c-kit, R&D cat #DYC3527-5
Anti-pY-HIRP antibody: R&D cat #DYC3527-5
25× wash buffer: Quantikine ELISA Wash Buffer 1, R&D cat #WA126
PBS: R&D cat #DY006
96 well ELISA plates: Clear Polystyrene Microplates, R&D Cat #DY990
96 well Tissue culture plates: Corning® 96 Well TC-Treated Microplates, cat #CLS3894
RPMI medium: ATCC® RPMI-1640 Medium, Cat #30-2001
FBS: Fetal Bovine Serum, certified, United States Gibco, Thermo-Fisher 16000044
GM-CSF: Recombinant Human GM-CSF Protein, R&D cat #215-GM
SCF: Recombinant Human SCF Protein, R&D cat #255-SC

### Protocol:

1. Prepare cells:
   a. Culture M-07e cells in RPMI medium+20% serum+GM-CSF+penstrep
      To make 50 mL of growth medium
         i. 40 mL RPMI media
         ii. 10 mL FBS
         iii. 10 uL of GM-CSF (100 ug/mL stock)
         iv. 0.5 mL pen strep (100×)
   b. Harvest cells
   c. Re-suspend cells in 100% RPMI media (without serum) to final concentration of 1 million cells/mL
   d. Transfer 100 uL of cell solution to 11 columns of a 96 well flat bottom cell culture plate
   e. Incubate cells over night at 37 C
2. Prepare ELISA Plates:
   a. Dilute capture antibody to concentration 4 ug/mL in PBS
   b. Coat 96 well microplates with 100 uL of capture antibody
   c. Seal plates and incubate at RT overnight
   d. Wash plates with 300 uL of wash buffer
   e. Repeat wash
   f. Treat plates with 300 uL of blocking buffer at RT for 1 hour
3. Addition of compounds:
   a. Prepare compound dilutions in RPMI media
      i. Top test concentration is 1 uM
      ii. Serially dilute compounds 3× for a total of 8 concentrations
   b. Add 10 uL of 10× compound dilutions to cells
      i. No compounds added in column 11 (for controls)
   c. Incubate cells with compounds for 2 hours at 37 C
   d. Stimulate cells with SCF for 5 min at concentration of 100 ng/mL (final)
      i. In column 11 stimulate top 4 wells. Leave bottom 4 wells unstimulated
   e. Remove cell culture media with multichannel pipet
   f. Add 110 uL of Lysis buffer to all wells
   g. Incubate plates at RT for 30 min while shaking
4. Transfer 100 uL of cell lysis to each prepared Elisa plate
5. Add 100 uL of the standard pc-KIT dilutions to the standard wells in column 12
   a. Top concentration=2000 pg/mL
   b. 2× dilutions
6. Incubate ELISA plate with cell lysate for 2 hours at RT
7. Wash Plate with wash buffer (wash 1)
8. Repeat wash (wash 2)
9. Repeat wash (wash 3) (do not allow plate to dry)
10. Add 100 uL of diluted anti-pY-HRP (1/1000 dilution in detection antibody dilution buffer) and incubate 2 hours at RT
11. Wash plate with wash buffer (wash 1)
12. Repeat wash (wash 2)
13. Repeat wash (wash 3)
14. Add 100 uL of substrate solution to each well
15. Incubate plates 20 min
   Note: Plate development is indicated by aqua color
16. Add 50 uL of stop solution to each well
17. Determine optical density on Bitek Synergy Neo2:
   a. Make new experiment

Select absorbance 1 set to 450 nm

| | |
|---|---|
| Buffer | |
| PBS | Use PBS from R&D for capture antibody dilution |
| Wash Buffer | 25x wash concentrate (R&D) diluted to 1x buffer in H2O |
| Block Buffer | 2% BSA in PBS |
| Lysis buffer | In DI water |
| | 1% NP-40 Alternative |
| | 20 mM Tris 7.5 |
| | 137 mM NaCl |
| | 10% Glycerol |
| | 2 mM EDTA |
| | 1 mM Sodium Orthovanadate |
| | Complete protease inhibitor (Roche) (2 tab/20 mL) |
| | Phosphostop (Roche)(2 tabs/20 mL) |
| Detection | In DI water |
| antibody | 20 mM TRIS, pH 7.3 |
| dilution | 137 mM NaCl |
| buffer | 0.05% Tween 20 |
| | 0.1% BSA |

### Part II—Results

Experimental results are provided in Table 7, below. The symbol “++++” indicates an IC₅₀ less than or equal to 0.05 μM. The symbol “+++” indicates an IC₅₀ in the range of greater than 0.05 μM and less than or equal to 0.5 μM. The symbol “++” indicates an IC₅₀ in the range of greater than 0.5 μM and less than or equal to 5 μM. The symbol “+” indicates an IC₅₀ in the range of greater than 5 μM to 30 μM.

TABLE 7

| Compound | M-07e pKIT |
|---|---|
| No. | IC₅₀ (μM) |
| I-71 | ++++ |
| I-72 | ++++ |
| I-73 | ++++ |
| I-75 | ++++ |
| I-76 | ++++ |
| I-77 | ++++ |
| I-78 | ++++ |
| I-79 | ++++ |
| I-80 | ++++ |
| I-81 | ++++ |
| I-82 | +++ |
| I-83 | ++++ |
| I-84 | ++++ |
| I-85 | ++++ |
| I-86 | ++++ |
| I-87 | ++++ |
| I-88 | ++++ |
| I-89 | ++++ |
| I-90 | ++++ |
| I-91 | ++++ |
| I-92 | ++++ |
| I-93 | ++++ |
| I-94 | ++++ |
| I-95 | ++++ |
| I-96 | ++++ |
| I-97 | ++++ |
| I-98 | ++++ |
| I-99 | ++++ |
| I-100 | ++++ |
| I-101 | +++ |
| I-102 | ++++ |
| I-103 | +++ |
| I-104 | ++++ |
| I-105 | +++ |
| I-106 | +++ |
| I-107 | ++++ |
| I-108 | ++++ |
| I-109 | ++++ |
| I-110 | +++ |
| I-111 | ++++ |
| I-112 | ++++ |
| I-113 | ++++ |
| I-122 | ++++ |
| I-114 | ++++ |
| I-115 | +++ |
| I-116 | +++ |
| I-117 | ++++ |
| I-119 | ++++ |
| I-121 | ++++ |
| I-120 | ++++ |
| I-123 | ++++ |
| I-124 | ++++ |
| I-125 | ++++ |
| I-126 | ++++ |
| I-127 | ++++ |
| I-131 | +++ |
| I-134 | ++ |
| I-136 | ++++ |
| I-138 | ++++ |
| I-140 | +++ |
| I-141 | ++++ |
| I-142 | ++++ |
| I-144 | ++++ |
| I-148 | ++++ |
| I-149 | ++++ |
| I-151 | ++++ |
| I-154 | ++++ |
| I-155 | +++ |
| I-157 | ++++ |
| I-158 | ++++ |
| I-428 | ++++ |
| I-167 | ++++ |
| I-173 | ++++ |
| I-429 | +++ |
| I-184 | ++ |
| I-185 | ++++ |
| I-190 | ++++ |
| I-191 | ++++ |
| I-195 | ++++ |
| I-204 | +++ |
| I-205 | ++++ |
| I-213 | +++ |
| I-214 | +++ |
| I-216 | ++++ |
| I-217 | +++ |
| I-230 | ++++ |
| I-235 | +++ |
| I-237 | +++ |
| I-238 | +++ |
| I-242 | +++ |
| I-251 | ++++ |
| I-255 | +++ |
| I-259 | ++++ |
| I-261 | ++++ |
| I-262 | ++++ |
| I-263 | ++++ |
| I-266 | ++++ |
| I-267 | ++++ |
| I-271 | ++++ |
| I-280 | ++++ |
| I-288 | +++ |
| I-296 | ++++ |
| I-306 | ++++ |
| I-314 | ++++ |
| I-319 | ++++ |
| I-336 | ++++ |
| I-337 | ++++ |
| I-359 | +++ |
| I-360 | ++++ |
| I-367 | ++++ |
| I-370 | ++++ |
| I-373 | ++++ |
| I-376 | ++++ |
| I-377 | ++++ |
| I-381 | ++++ |
| I-387 | ++++ |
| I-393 | ++++ |
| I-118 | +++ |
| I-160 | +++ |
| I-162 | +++ |
| I-164 | ++++ |
| I-172 | ++ |
| I-175 | +++ |
| I-212 | +++ |
| I-220 | +++ |
| I-221 | +++ |
| I-225 | ++++ |
| I-240 | +++ |
| I-269 | +++ |
| I-281 | ++++ |
| I-339 | ++++ |
| I-341 | +++ |

### Example 99—Permeability Assays and/or Inhibition Assays and/or Efflux Assays in Caco-2, MDCKII-MDR1, and MDCKII-BCRP Cell Lines

Exemplary compounds were tested for cell permeability in Caco-2, MDCKII-MDR1 and MDCKII-BCRP cell lines with efflux transporter engagement inferred from the efflux ratios determined. Procedures and results are described below. As described above and herein, it has been surprisingly found that certain compounds of the present invention have reduced brain penetration. This feature of certain compounds is in part due to efflux transporter engagement. See, for instance, exemplary such compounds included in data tables below.

### Part I—Procedures for Assays

### 5. Caco-2 Permeability Assay

### Methodology

### Materials

corning 96-well insert plate Cat:351131
corning 96-well acceptor plate Cat:353925

### Caco-2 Culture

Caco-2 cells purchased from ATCC were seeded onto polyethylene membranes (PET) in 96-well Corning Insert plates at 1×105 cells/cm², and refreshed medium every 4˜5 days until to the 21^{st} to 28^{th} day for confluent cell monolayer formation.

### Experimental Procedures

The transport buffer in the study was HBSS with 10.0 mM HEPES at pH 7.40±0.05. Test compound was tested at 2.00 μM bi-directionally in duplicate. Digoxin was tested at 10.0 μM bi-directionally in duplicate, while nadolol and metoprolol were tested at 2.00 μM in A to B direction in duplicate. Final DMSO concentration was adjusted to less than 1%. The plate was incubated for 2 hours in CO2 incubator at 37±1° C., with 5% CO2 at saturated humidity without shaking. And all samples after mixed with acetonitrile containing internal standard were centrifuged at 3200×g for 10 min. For nadolol and metoprolol, 200 μL supernatant solution was diluted with 600 μL ultra-pure water for LC-MS/MS analysis. For digoxin and test compounds, 200 μL supernatant solution was diluted with 200 μL ultra-pure water for LC-MS/MS analysis. Concentrations of test and control compounds in starting solution, donor solution, and receiver solution were quantified by LC-MS/MS methodologies, using peak area ratio of analyte/internal standard.

After transport assay, lucifer yellow rejection assay was applied to determine the Caco-2 cell monolayer integrity.

After transport assay, Lucifer yellow rejection assay was applied to determine the Caco-2 cell monolayer integrity. And permeation of lucifer yellow through the monolayer was measured to evaluate the cellular integrity.

### Data Analysis

The apparent permeability coefficient Pₐₚₚ (cm/s) was calculated using the equation: Paₚₚ=(dCᵣ/dt)×Vr/(A×C₀), where dCᵣ/dt is the cumulative concentration of compound in the receiver chamber as a function of time (μM/s); Vᵣ is the solution volume in the receiver chamber (0.075 mL on the apical side, 0.25 mL on the basolateral side); A is the surface area for the transport, i.e. 0.0804 cm² for the area of the monolayer; C₀ is the initial concentration in the donor chamber (μM).

The efflux ratio was calculated using the equation: Efflux Ratio=Pₐₚₚ (BA)/Pₐₚₚ(AB).

Percent recovery was calculated using the equation: % Solution Recovery=100×[(Vᵣ×Cᵣ)+(V_{d}×C_{d})]/(V_{d}×C₀), where V_{d} is the volume in the donor chambers (0.075 mL on the apical side, 0.25 mL on the basolateral side); C_{d} and Cᵣ are the final concentrations of transport compound in donor and receiver chambers, respectively.

### B. MDCKII-BCRP Assay

The reference compound Teriflunomide and test compounds are tested in the bidirectional assay. Test compound and reference compounds incubations were tested in duplicates.

The transport assay can be performed between days 12 to day 15 using ready to use PreadyPort™ BCRP-MDCKII monolayer in 96 well plate.

PreadyPort™ plate was removed from the incubator and placed in the laminar flow hood.

The medium was aspirated from the apical and basal compartments and 75 and 235 μL assay buffer was added to each of the 96 wells of apical and basal compartments of the PreadyPort™ plate.

The plates were pre-incubated for 15 min at 37° C. with 5% CO₂ in humidified atmosphere. After preincubation the assay buffer was removed from the apical and basal inserts of the plate.

In apical to basolateral (A→B) transport, 75 μL of test or reference compound working solution to apical inserts and 235 μL of blank assay buffer were added to the corresponding basal compartments.

In basolateral to apical transport, 235 μL of test or reference compound working solution to the basal compartments and 75 μL of blank assay buffer solution were added to corresponding apical inserts.

The sandwich plate was incubated for 120 min in incubator at 5% CO₂ and 37° C.

### Sample Processing

After incubation, 60 μL was transferred from receiver and donor compartments to collection plate.

For donor and C₀ samples 20-fold dilution was accomplished by mixing 60 μL of collected sample with 1140 μL of transport buffer and from that solution aliquot 60 μL transfer Both receiver and donor samples were processed by adding 5 volumes of acetonitrile containing internal standard (60 μL of sample+240 μL of acetonitrile containing glyburide as internal standard, 125 ng/mL) and vortexing the samples. The compound concentration was measured in all samples using respective calibration standards by LC-MS/MS.

### Note:

Any changes in the procedure should be captured in LNB.
Replicates and test concentration can be only modified with due approval of the group leader.
Choice of buffers, pH, and temperature can be modified as requested by the end user.

### Data Analysis

Calculate the Pₐₚₚ and recovery using the given formula:

$$Permeability ( cm / s ) : {P}_{app} = \frac{d Q}{d t} \times \frac{V}{{C}_{0}} \times \frac{1}{A}$$

$$Recovery \% = 100 \times \frac{( donor volume \times Cend , d ) + ( acceptorvolume \times Cend , a )}{donor volume \times C HBSS}$$

Where: dQ/dt=Permeability rate in μM/see
   C₀=Initial concentration in μM
   A=Membrane surface area (0.33 cm²)
   V=Volume of receiver chamber
   C_{end, a}=concentration in acceptor well at end of incubation
   C_{end, d}=concentration in donor well at end of incubation
   C_{HBSS}=loading concentration measured in HBSS

### Classification of Transport is as Follows:

| | |
|---|---|
| <2 × 10⁻⁶ cm/sec | Low permeability |
| 2 × 10⁻⁶ to 20 × 10⁻⁶ cm/sec | Medium permeability |
| >20 × 10⁻⁶ cm/sec | High permeability |

### Calculation for Efflux Ratio:

$$Efflux ratio = \frac{{Pe}_{app} B \to A}{{Pe}_{app} A \to B}$$

### Criteria for Efflux Ratio:

| | |
|---|---|
| <2 | Non efflux Substrate |
| 2-5 | Mild efflux Substrate |
| >5 | Efflux Substrate |

### LYR:

$$\% Translocation of Lucifer Yellow = \frac{R F U}{R F U dosing solution} \times 100$$

### C. Permeability in MDR1-MDCKII Cell Protocol

MDR1-MDCKII cells (obtained from Piet Borst at the Netherlands Cancer Institute) were seeded onto polyethylene membranes (PET) in 96-well insert systems at 2.5×10⁵ cells/mL until to 4-7 days for confluent cell monolayer formation.

Test compounds were diluted with the transport buffer (HBSS with 10 mM Hepes, pH7.4) from DMSO stock solution to a concentration of 2.00 μM (DMSO<1%) and applied to the apical or basolateral side of the cell monolayer. Permeation of the test compounds from A to B direction or B to A direction was determined in duplicate. Digoxin was tested at 10.0 μM from A to B direction or B to A direction as well, while nadolol and metoprolol were tested at 2.00 M in A to B direction in duplicate. The plate was incubated for 2.5 hours in CO2 incubator at 37±1° C., with 5% CO2 at saturated humidity without shaking. In addition, the efflux ratio of each compound was also determined. Test and reference compounds were quantified by LC-MS/MS analysis based on the peak area ratio of analyte/IS.

After transport assay, Lucifer yellow rejection assay are applied to determine the cell monolayer integrity. Buffers are removed from both apical and basolateral chambers, followed by the addition of 75 μL of 100 μM lucifer yellow in transport buffer and 250 μL transport buffer in apical and basolateral chambers, respectively. The plate is incubated for 30 minutes at 37° C. with 5% CO2 and saturated humidity without shaking. After 30 minutes incubation, 20 μL of lucifer yellow samples are taken from the apical sides, followed by the addition of 60 μL of Transport Buffer. And then 80 μL of lucifer yellow samples are taken from the basolateral sides. The relative fluorescence unit (RFU) of lucifer yellow is measured at 425/528 nm (excitation/emission) with an Envision plate reader.

### Data Analysis

The apparent permeability coefficient Pₐₚₚ (cm/s) was calculated using the equation:

$${P}_{app} = ( {dC}_{r} / dt ) \times {V}_{r} / ( A \times {C}_{0} )$$

Where dCᵣ/dt is the cumulative concentration of compound in the receiver chamber as a function of time (μM/s); Vᵣ is the solution volume in the receiver chamber (0.075 mL on the apical side, 0.25 mL on the basolateral side); A is the surface area for the transport, i.e. 0.0804 cm² for the area of the monolayer; C₀ is the initial concentration in the donor chamber (μM). The efflux ratio was calculated using the equation:

$$Efflux Ratio = {P}_{app} ( BA ) / {P}_{app} ( AB )$$

### Percent Recovery was Calculated Using the Equation:

$$\% Recovery = 100 \times [ ( {V}_{r} \times {C}_{r} ) + ( {V}_{d} \times {C}_{d} ) ] / ( {V}_{d} \times {C}_{0} )$$

Where V_{d} is the volume in the donor chambers (0.075 mL on the apical side, 0.25 mL on the basolateral side); C_{d} and Cᵣ are the final concentrations of transport compound in donor and receiver chambers, respectively.

### Part II—Results

TABLE 8

| | | | MDCKII- | MDCKII- | MDCK- | MDCK- |
|---|---|---|---|---|---|---|
| | Caco-2: | Caco-2: | MDR1: | MDR1: | BCRP: | BCRP: |
| Compound | Pₐₚₚ A-B | Efflux | Pₐₚₚ A-B | Efflux | Pₐₚₚ A-B | Efflux |
| No. | (10⁻⁶ cm/s) | Ratio | (10⁻⁶ cm/s) | Ratio | (10⁻⁶ cm/s) | Ratio |
| I-71 | 6.65 | 6.78 | 5.94 | 5.15 | | |
| I-83 | | | | | 0.96 | 25.3 |
| I-84 | 11.8 | 1.8 | 12.1 | 1.78 | 20.7 | 2.28 |
| I-87 | 7.81 | 0.66 | 4.58 | 0.74 | 30.4 | 0.71 |
| I-88 | 6.24 | 0.79 | 3.92 | 0.86 | 29 | 0.89 |
| I-90 | | | | | 2.89 | 1.64 |
| I-91 | | | | | 1.11 | 1.76 |
| I-93 | | | | | 21 | 1.16 |
| I-94 | | | | | 5.19 | 8.04 |
| I-96 | | | | | 8.58 | 1.44 |
| I-434 | | | | | 15.4 | 1.1 |
| I-435 | | | | | 16.8 | 1.02 |
| I-109 | | | | | 88.7 | 1.12 |
| I-112 | | | | | 6.8 | 4.01 |
| I-113 | | | | | 15.5 | 1.28 |
| I-436 | | | | | 15.5 | 1.6 |
| I-400 | | | | | 11.7 | 1.44 |
| I-437 | | | | | 10 | 1.46 |
| I-438 | | | | | 7.56 | 1.78 |
| I-122 | | | | | 1.63 | 29.4 |
| I-114 | 6.74 | 4.4 | 7.8 | 3.67 | 3.03 | 8.07 |
| I-123 | | | | | 12.4 | 0.94 |
| I-124 | | | | | 9.07 | 1.14 |
| I-125 | | | | | 9.07 | 1.6 |
| I-134 | | | | | 7.85 | 2.12 |
| I-138 | 16.6 | 1.42 | 22 | 1.01 | 10.4 | 5.11 |
| I-148 | 8.17 | 2.85 | 11.7 | 1.45 | 4.44 | 7.82 |
| I-149 | 12.5 | 0.73 | 12.1 | 0.71 | 9.33 | 1.56 |
| I-154 | 5.78 | 5.15 | 9.15 | 2.24 | | |
| I-157 | 6.65 | 1.87 | 14.3 | 1.9 | 2.22 | 14.8 |
| I-158 | 6 | 1.8 | 15.3 | 1.83 | 3.33 | 10.7 |
| I-167 | 18.1 | 0.44 | 22 | 0.73 | | |
| I-173 | 15.9 | 0.68 | 14.8 | 0.64 | 12.6 | 0.92 |
| I-204 | | | | | 3.27 | 6.11 |
| I-205 | | | | | 23.5 | 1.66 |
| I-211 | | | | | 1.91 | 14.1 |
| I-213 | | | | | 20.6 | 1.42 |
| I-216 | | | | | 0.49 | 43.3 |
| I-217 | | | | | 0.86 | 28.1 |
| I-219 | | | | | 0.79 | 37 |
| I-228 | | | | | 1.08 | 22.9 |
| I-235 | | | | | 3.11 | 6.86 |
| I-237 | 0.50 | 57.6 | 1.43 | 20.5 | 1.66 | 11.5 |
| I-238 | | | | | 0.92 | 22.7 |
| I-246 | | | | | 1.81 | 16.9 |
| I-251 | 12 | 2.37 | 11 | 2.28 | 8.23 | 2.55 |
| I-252 | | | | | 11.1 | 1.96 |
| I-256 | | | | | 3.76 | 9.12 |
| I-257 | | | | | 1.26 | 28.5 |
| I-258 | | | | | 1.17 | 25.7 |
| I-259 | | | | | 0.56 | 27.5 |
| I-260 | | | | | 1.54 | 8.45 |
| I-262 | | | | | 0.54 | 46.5 |
| I-263 | | | | | 0.56 | 45.4 |
| I-264 | | | | | 0.71 | 24.2 |
| I-265 | | | | | 1 | 19.4 |
| I-267 | | | | | 7.77 | 2.66 |
| I-268 | | | | | 0.31 | 11.6 |
| I-270 | 8.5 | 3.35 | 17.2 | 1.73 | 1.3 | 20.8 |
| I-271 | 6.26 | 2.93 | 18.5 | 1.36 | 1.25 | 29.6 |
| I-272 | | | | | 6.58 | 3.4 |
| I-273 | | | | | 20.3 | 0.56 |
| I-274 | | | | | 0.000847 | 71.8 |
| I-277 | | | | | 0.24 | 7.82 |
| I-278 | 0.51 | 3.14 | 0.51 | 1.56 | | |
| I-279 | 0.21 | 109 | 1.5 | 23.8 | 0.22 | 31.6 |
| I-280 | 14 | 0.86 | 11.8 | 1.19 | 5.07 | 2.55 |
| I-282 | 0.14 | 152 | 0.47 | 70.3 | 0.55 | 28.1 |
| I-287 | 0.13 | 135 | 0.83 | 27.9 | 11.8 | 0.65 |
| I-288 | 17.4 | 0.56 | 20.6 | 0.51 | 7.81 | 1.01 |
| I-293 | | | | | 0.02 | 4.55 |
| I-295 | | | | | 6.12 | 1.58 |
| I-296 | 1.83 | 19.2 | 3.61 | 12.7 | 1.08 | 42.5 |
| I-298 | | | | | 0.58 | 28.7 |
| I-299 | | | | | 0.01 | 13.7 |
| I-304 | 4.31 | 0.92 | 3.65 | 1.08 | 5.05 | 3.43 |
| I-306 | 7.03 | 3.66 | 14.5 | 2.88 | 1.46 | 23.2 |
| I-307 | 9.28 | 1.79 | 8.84 | 1.93 | 7.39 | 3.77 |
| I-308 | | | | | 16.3 | 1.13 |
| I-309 | | | | | 11.3 | 1.07 |
| I-310 | | | | | 12 | 0.81 |
| I-312 | | | | | 13.3 | 1.42 |
| I-313 | | | | | 6.89 | 3.44 |
| I-314 | 11.6 | 2.42 | 17.7 | 1.35 | 7.64 | 3.83 |
| I-316 | | | | | 14.7 | 1.03 |
| I-318 | | | | | 13.9 | 0.93 |
| I-319 | 16 | 0.83 | 10 | 0.76 | 13.1 | 1.02 |
| I-320 | | | | | 1.75 | 16 |
| I-321 | | | | | 4.73 | 3.4 |
| I-322 | | | | | 1.38 | 2.7 |
| I-325 | | | | | 11.4 | 1.39 |
| I-326 | | | | | 1.07 | 28.3 |
| I-327 | 0.27 | 102 | 0.78 | 38.7 | 0.28 | 73.2 |
| I-328 | 18.2 | 1.02 | 20.8 | 0.93 | 6.06 | 4.55 |
| I-329 | | | | | 0.22 | 40.6 |
| I-330 | | | | | 1.28 | 24.9 |
| I-335 | | | | | 1.99 | 0.37 |
| I-336 | 9.39 | 2.62 | 15.4 | 1.85 | 1.92 | 19.9 |
| I-337 | | | | | 3.73 | 4.71 |
| I-342 | | | | | 30 | 0.47 |
| I-343 | | | | | 8.04 | 2.36 |
| I-344 | | | | | 11.5 | 1.48 |
| I-346 | | | | | 40.1 | 1.33 |
| I-347 | | | | | 0.17 | 1.28 |
| I-348 | | | | | 0.13 | 2.37 |
| I-349 | | | | | 16.6 | 2.56 |
| I-350 | | | | | 8.86 | 1.52 |
| I-353 | | | | | 0.28 | 1.31 |
| I-354 | | | | | 7.97 | 1.03 |
| I-355 | | | | | 12.3 | 0.91 |
| I-357 | | | | | 0.47 | 63.9 |
| I-358 | | | | | 7.81 | 3.69 |
| I-359 | 9.88 | 1.19 | 21.4 | 0.81 | 4.68 | 7.14 |
| I-360 | 12.7 | 0.36 | | | 9.54 | 1.52 |
| I-361 | | | | | 1.1 | 0.54 |
| I-362 | | | | | 8.71 | 1.35 |
| I-363 | 8.85 | 2.55 | 13 | 2.01 | 3.24 | 5.58 |
| I-364 | 8.39 | 3.09 | 16 | 2.02 | 2.19 | 9.18 |
| I-365 | | | | | 4.73 | 1.21 |
| I-366 | | | | | 10.2 | 1.31 |
| I-367 | | | | | 0.83 | 1.18 |
| I-368 | | | | | 15 | 1.09 |
| I-369 | 20.6 | 0.77 | 24.4 | 0.87 | 8.93 | 1.62 |
| I-370 | 11.8 | 2.17 | 17 | 1.05 | 5.69 | 3.97 |
| I-371 | 12 | 1.36 | 15.9 | 0.78 | 6.7 | 2.33 |
| I-373 | 15.2 | 2.02 | | | 4.51 | 4.48 |
| I-375 | | | | | 11 | 1.33 |
| I-376 | 19.5 | 0.85 | 8.41 | 0.54 | 6.24 | 1.02 |
| I-377 | | | | | 7.81 | 0.98 |
| I-378 | | | | | 16 | 0.77 |
| I-380 | 7.5 | 3.45 | 10.9 | 2.34 | 1.21 | 14.2 |
| I-381 | 17.1 | 0.97 | 5.95 | 0.55 | 10.1 | 1.25 |
| I-382 | | | | | 8.32 | 1.04 |
| I-383 | | | | | 7.55 | 1.74 |
| I-384 | 16.5 | 0.90 | 17.9 | 0.88 | 0.59 | 12.9 |
| I-385 | | | | | 14.3 | 1.05 |
| I-386 | | | | | 11.4 | 1.56 |
| I-387 | 14.5 | 0.77 | 7.59 | 0.48 | 13.8 | 0.82 |
| I-388 | | | | | 0.4 | 38.9 |
| I-389 | 1.25 | 25.4 | 3.53 | 10.9 | 0.87 | 54.3 |
| I-390 | | | | | 12.9 | 14.9 |
| I-391 | | | | | 2.13 | 12.1 |
| I-392 | | | | | 7.51 | 2.82 |
| I-393 | 18.1 | 0.81 | 4.66 | 0.51 | 9.64 | 1.57 |
| I-394 | 6.31 | 3.48 | 6.21 | 3.08 | 1.42 | 23.2 |
| I-395 | | | | | 19 | 3.28 |
| I-396 | | | | | 8.25 | 2.86 |
| I-397 | | | | | 8.04 | 2.83 |
| I-398 | | | | | 20.3 | 1.33 |
| I-399 | | | | | 6.48 | 2.78 |
| I-162 | 12.6 | 0.67 | 16.2 | 0.63 | | |
| I-172 | 12.9 | 0.49 | 22.7 | 0.46 | 6.02 | 0.81 |
| I-175 | 21.9 | 0.33 | 26.2 | 0.58 | | |
| I-220 | 16.5 | 0.25 | 16.3 | 0.71 | 26.4 | 1.17 |
| I-225 | 9.62 | 1.37 | 15.9 | 1.1 | 11.2 | 3.27 |
| I-240 | | | | | 14.5 | 2 |
| I-269 | 27.4 | 0.55 | 23.6 | 0.67 | 17.7 | 0.72 |
| I-281 | 18.9 | 0.82 | 24.1 | 0.86 | 18.9 | 1.32 |
| I-332 | | | | | 2.63 | 1.75 |
| I-338 | | | | | 23.1 | 0.82 |
| I-339 | 22.2 | 0.56 | 22.8 | 0.84 | 13 | 1.75 |
| I-340 | | | | | 80.4 | 0.28 |
| I-341 | 16.2 | 0.56 | 17.7 | 0.82 | 10.1 | 0.84 |

### Example 100—Three Time Point Assay for Plasma, Brain and Testes Distribution

Exemplary compounds were tested for distribution in plasma, brain and testes tissue following oral dosing of the compounds in rats. Procedures and results are described below.

### Part I—Procedures for Three Time Point Assay for Plasma, Brain and Testes Distribution

### Animal Husbandry:

Male SD Rats were group-housed (up to four animals/sex/cage) in polysulfone cages with certified aspen wood bedding during acclimation and study period.

Environment controls were set to maintain a temperature range of 20-26° C., a relative humidity range of 40 to 70%, and a 12-hour light/12-hour dark cycle. The light/dark cycle was interrupted as needed for study-related activities. The temperature and relative humidity were continuous monitored by Vaisala ViewLinc Monitoring system.

### Animal Fasting Detail Information:

Certified rodent diet and water was provided to all animals ad libitum, unless fasting for study procedures.

Water was autoclaved before it was provided to the animals. Water samples were periodically analyzed by a certified laboratory for specified microorganisms and environment contaminants. The diet was routinely analyzed by the manufacturer for specified microorganisms, nutritional components and environmental contaminants. Results were reviewed and assessed by veterinary staff and archived.

### Dose Formulation

### Formulation for PO:

Appropriate amount of test article was accurately weighed and mixed with an appropriate volume of vehicle to get a clear solution or a uniform suspension; vortexing or sonication in water bath may also be need. Animals were dosed within four hours after the formulation is prepared.

Formulation samples were removed from each of the formulation solutions or suspensions, transferred into 1.5 mL of polypropylene microcentrifuge tubes and run dose validation by LC/UV or LC-MS/MS.

### Dose Administration:

For PO dosing, the dose formulation was administered via oral gavage following facility SOPs. The dose volume was determined by the animals' body weight collected on the morning of dosing day. The dose was 10 mg/kg.

### Sample Collection

### Blood Collection:

Each blood collection (about 1 mL per time point at one or more of 0.5 h, 2 h, 3 h, 5.3 h, and 7 h) was performed from jugular vein or other suitable site of each animal into pre-chilled commercial EDTA-K2 tubes and placed on wet ice until centrifugation.

### Plasma Processing:

Blood samples were processed for plasma by centrifugation at approximately 4° C., 3,200 g for 10 min. Plasma was collected respectively and transferred into pre-labeled 96 well plate or polypropylene tubes, quick frozen over dry ice and kept at −60° C. or lower until LC-MS/MS analysis.

### Brain and Testis Tissue Processing:

Rat brain was perfused with PBS to remove blood before harvest.

After perfusion, brain and testis were homogenized using homogenizing buffer (15 mM PBS (pH7.4):MeOH=2:1) at the ratio of 1:9 (1 g tissue with 9 mL buffer, the dilution ratio is 10). The brain homogenate was kept at −60° C. or lower until LC-MS/MS analysis. General sample processing procedure (brain homogenate & testis homogenate) using 96-well plate:
1). An aliquot of 40 μL unknown sample, calibration standard, quality control and dilution quality control (if have), single blank, and double blank sample was added to the 96-well plate respectively;
2). Each sample (except the double blank) was quenched with 400 μL of IS1 respectively (double blank sample was quenched with 400 μL of I), and then the mixture was vortex-mixed for 10 min at 800 rpm and centrifuged for 15 min at 3220×g, 4° C.;
3). An aliquot of 50 μL supernatant was transferred to another clean 96-well plate and centrifuged for 5 min at 3220×g, 4° C., then the supernatant was directly injected for LC-MS/MS analysis.

### Clinical Observations

All animals were observed at dosing and each scheduled collection. All abnormalities were recorded.

### Dose Formulation Concentration Verification

A LC-UV or LC-MS/MS method was developed with a calibration curve consisting of 6 calibration standards.
The concentrations of the test compound in dose formulation samples were determined by the LC-UV or LC-MS/MS method.
Acceptance criteria for an analytical run: at least of 5 of 6 calibration standards should be within +20% of nominal values by using LC-UV method and ±30% of nominal values by using LC-MS/MS method.

### Bioanalytical Method Development and Sample Analysis

### LC-MS/MS Method Development:

1. A LC-MS/MS method for the quantitative determination of test compound in biological matrix was developed under Non-GLP compliance.
2. A calibration curve with at least 6 non-zero calibration standards was applied for the method including LLOQ.
3. A set of QCs consisting of low, middle, and high concentrations were applied for the method.
4. N in 1 cassette LC-MS/MS method was developed for samples coming from different studies and the interference among all cassette analytes was evaluated during the method development.
5. Cassette administration assay was performed if the mass difference (Δₘₐₛₛ) among different analytes was more than 4 Da. In this case, interference evaluation was not necessary.

If the Δₘₐₛₛ among different analytes was less than 4 Da, there was a potential risk that interference would occur during LC-MS/MS analysis. Interference among analytes was evaluated but the LC separation of those analytes by using a generic method was tried. If these analytes could not be separated, the experiment record was documented.
6. Biological sample in matrix other than plasma was diluted with plasma first and then quantified against plasma calibration curve. And the corresponding dilution QC to ensure the dilution accuracy and no significant matrix difference, was inserted into analytical run.

### Sample Analysis:

1. If sample number within a batch was ≤12, at least one set of standard curve separated with two parts through begin and end of the sequence, was included in the run, and QCs were not required. The recommended injection order was C8, C6, C4, C2, study samples, C7, C5, C3, C1.
2. If sample number within a batch was >12, one standard curve and two sets of QCs with low, middle and high concentrations were applied for bioanalysis. Meanwhile, QCs number should be more than 5% of study sample number.
3. Samples with the same types of matrix in different studies were quantified in one analysis run by using the developed N in 1 cassette LC-MS/MS method.
4. Biological samples in matrix other than plasma were diluted with plasma and then quantified against plasma calibration curve. The corresponding dilution QC to ensure the dilution accuracy and no significant matrix difference, will be inserted into analytical run. Optionally, biological samples were quantified against calibration curves in their own corresponding matrix.

### Acceptance Criteria:

1. Linearity: ≥75% STDs is back calculated to within ±20% of their nominal values in biofluid and within ±25% of their nominal values in tissue and feces sample. If the endpoints, such as LLOQ and ULOQ, on the calibration curve are eliminated, the calibration curve will be truncated. The truncated calibration curve should consist of at least 75% of the initial STDs.
2. Accuracy: ≥67% QCs is back calculated to within +20% of their nominal values for biofluid and within ±25% of their nominal values for tissue and feces samples.
3. Specificity: The mean calculated concentration in the single blank matrix should be ≤50% LLOQ.
4. Sensitivity:
   4.1 If the biological samples in matrix other than plasma were diluted with plasma and quantified against plasma calibration curve, the LLOQ of plasma calibration curve were targeted to ≤2 ng/mL, which LLOQ is equivalent to ≤4 ng/mL in biological matrix other than plasma (if dilution 2 folds is applied).
   4.2 If the biological samples were quantified against the calibration curves prepared by their corresponding matrix, the LLOQ was targeted to ≤3 ng/mL.
5. Carryover: The mean calculated carry-over peak area in the blank matrix immediately after the highest standard injection should be less than that of LLOQ. If the carryover couldn't meet the criteria, the impact of the carryover on unknown samples should be evaluated according to the below procedure:

Carryover evaluation should be estimated according to absolute carryover. Carryover contribution is calculated by the area ratio of the blank with the highest carryover (Area max of carryover blank) to the ULOQ with the minimum calculated value (Area min of ULOQ); Carryover impact is calculated by the area ratio of one injection (Area of one injection) to the following injection (Area of the following injection); Absolute carryover is calculated by carryover contribution multiplies carryover impact, the value of absolute carryover should be below the acceptable accuracy of the studies (e.g., 200 or 25K).

$$Carryover contribution = Areamax of carryover blank / Areamin of ULOQ$$

$$Carryover impact = Area of one injection / Area of the following injection$$

$$Absolute carryover = Carryover contribution * Carryover impact$$

### Requirements

### Part II—Results

TABLE 9

| | Time | Plasma | Brain | Testes | | |
|---|---|---|---|---|---|---|
| Compound | point | concentration | concentration | concentration | Kₚ | Kₚ |
| No. | (h) | (ng/ml) | (ng/g) | (ng/g) | brain/plasma | testes/plasma |
| I-40 | 0.5 | 712 | 63.2 | 123 | 0.09 | 0.17 |
| | 2 | 1770 | 144 | 568 | 0.08 | 0.32 |
| | 7 | 764 | 65.4 | 300 | 0.09 | 0.41 |
| I-63 | 0.5 | 1750 | 350 | 355 | 0.21 | 0.21 |
| | 2 | 4320 | 810 | 1080 | 0.19 | 0.25 |
| | 7 | 4020 | 1170 | 1300 | 0.30 | 0.35 |
| I-71 | 0.5 | 1810 | 91.8 | 166 | 0.05 | 0.09 |
| | 2 | 3420 | 169 | 653 | 0.05 | 0.19 |
| | 7 | 4110 | 126 | 739 | 0.03 | 0.18 |
| I-87 | 0.5 | 454 | 248 | 159 | 0.60 | 0.38 |
| | 2 | 1480 | 954 | 989 | 0.68 | 0.71 |
| | 7 | 4970 | 1730 | 1650 | 0.36 | 0.34 |
| I-88 | 0.5 | 2640 | 722 | 480 | 0.27 | 0.19 |
| | 2 | 8390 | 1200 | 1330 | 0.14 | 0.16 |
| | 7 | 9200 | 1250 | 1950 | 0.15 | 0.21 |
| I-114 | 0.5 | 1540 | 106 | 256 | 0.07 | 0.17 |
| | 2 | 1680 | 113 | 482 | 0.07 | 0.29 |
| | 7 | 693 | 45.5 | 248 | 0.07 | 0.38 |
| I-296 | 0.5 | 1840 | 28 | 89.5 | 0.02 | 0.05 |
| | 2 | 2760 | 39 | 213 | 0.02 | 0.08 |
| | 7 | 1290 | 19 | 138 | 0.02 | 0.11 |
| I-432 | 0.5 | 800 | 366 | 312 | 0.46 | 0.40 |
| | 2 | 599 | 247 | 322 | 0.38 | 0.50 |
| | 7 | 102 | 35.3 | 46 | 0.34 | 0.52 |
| I-433 | 0.5 | 2350 | 1240 | 800 | 0.51 | 0.35 |
| | 2 | 2910 | 1330 | 1150 | 0.46 | 0.40 |
| | 7 | 3430 | 1580 | 1350 | 0.45 | 0.39 |
| I-76 | 3 | 598 | 55.9 | 246 | 0.09 | 0.41 |
| I-84 | 2 | 5900 | 400 | 655 | 0.07 | 0.11 |
| I-149 | 5.3 | 3220 | 1660 | 1620 | 0.52 | 0.50 |
| I-154 | 2 | 1370 | 143 | 683 | 0.11 | 0.50 |
| I-173 | 2 | 8980 | 4470 | 3240 | 0.50 | 0.36 |
| I-225 | 2 | 3420 | 506 | 1140 | 0.15 | 0.33 |
| I-237 | 2 | 1090 | 0 | 192 | 0 | 0.18 |
| I-238 | 2 | 791 | 0 | 131 | 0 | 0.17 |
| I-256 | 2 | 1880 | 105 | 477 | 0.06 | 0.25 |
| I-257 | 2 | 127 | 0 | 27.1 | 0 | 0.21 |
| I-258 | 2 | 746 | 0 | 62.8 | 0 | 0.11 |
| I-270 | 2 | 422 | 25.6 | 39.3 | 0.06 | 0.09 |
| I-271 | 2 | 1760 | 325 | 1020 | 0.18 | 0.58 |
| I-281 | 2 | 3980 | 768 | 1880 | 0.19 | 0.47 |
| I-295 | 2 | 2080 | 377 | 1020 | 0.18 | 0.49 |
| I-299 | 2 | 216 | 101 | 198 | 0.47 | 0.92 |
| I-307 | 2 | 389 | 82.4 | 464 | 0.21 | 1.19 |
| I-314 | 2 | 1920 | 289 | 732 | 0.15 | 0.38 |
| I-328 | 2 | 60.3 | 25.4 | 56.2 | 0.42 | 0.93 |
| I-330 | 2 | 117 | 0 | 100 | 0 | 0.86 |
| I-335 | 2 | 16.7 | 0 | 0 | 0 | 0 |
| I-339 | 2 | 1200 | 603 | 947 | 0.50 | 0.79 |
| I-340 | 2 | 2230 | 3660 | 3010 | 1.64 | 1.35 |
| I-359 | 2 | 2950 | 52.7 | 345 | 0.02 | 0.12 |
| I-360 | 2 | 3650 | 3420 | 2960 | 0.94 | 0.81 |
| I-363 | 2 | 815 | 46 | 104 | 0.06 | 0.13 |
| I-369 | 2 | 1630 | 1940 | 2060 | 1.19 | 1.27 |
| I-373 | 2 | 3680 | 845 | 1740 | 0.23 | 0.47 |
| I-380 | 2 | 477 | 28.2 | 44.8 | 0.06 | 0.09 |
| I-384 | 2 | 40.6 | 21.4 | 60.7 | 0.53 | 1.49 |
| I-389 | 2 | 2030 | 63 | 1830 | 0.03 | 0.91 |

### Example 101—Hepatocyte CYP Induction Assay

Exemplary compounds were tested for ability to induce CYP3A4 and/or CYP1A2 in hepatocytes. Procedures and results are described below.

### Part I—Procedures for CYP Induction Assay

### Materials and Methods

### Hepatocytes

Cryopreserved human hepatocytes were purchased from BioIVT (Baltimore, MD, USA), and they were stored in liquid nitrogen before using. The detailed information of hepatocytes is as follows:

### Control Compound

Positive control was purchased from commercial vender.

The detailed information is as follows:

| Reference | | | Catalog |
|---|---|---|---|
| Compound | Application | Source | Number |
| Rifampicin | positive control | Sigma-Aldrich | R3501 |

### Reagents and Consumables

| | | Catalog |
|---|---|---|
| Name | Source | Number |
| Acetonitrile | Merck Chemical | 1.00030.4008 |
| Dexamethasone | Sigma-Aldrich | D1756 |
| Dimethyl sulfoxide | Sigma-Aldrich | D2650 |
| Fetal bovine serum | AusgeneX | FBS500-S |
| HEPES Buffer Solution (1M) | Gibco | 15630-080 |
| Methanol | Merck Chemical | 1.06007.4008 |
| Matrigel | BD Biosciences | 4E+05 |
| HBSS | Gibco | 14025-076 |
| Incubation Medium | BioIVT | Z99009 |
| Plating Medium | BioIVT | S03316 |
| Penicillin & Streptomycin | HyClone | SV30010 |
| Opti THAW Hepatocyte media | Xenotech | K8000 |
| TaqMan universal PCR MasterMix | Applied Biosystems | 4E+06 |
| Eukaryotic 18S rRNA Endogenous | Applied Biosystems | 4319413E |
| Control | | |
| Human CYP3A4 20X Gene | Applied Biosystems | Hs00604506- |
| Expression Assay labeled with | | m1 |
| FAM/MGB | | |
| iScript cDNA Synthesis Kit | Bio-Rad | 2E+06 |
| Rneasy 96 QIAcube HT Kit | QIAGEN | 74171 |
| QIAcube HT Kit Plasticware | QIAGEN | 1E+06 |
| Lactate dehydrogenase cytotoxicity | Beyotime | C0016 |
| assay kit | | |
| Collagen-coated 96-well plates | Corning | 4E+05 |
| PCR Microplate | Axygen | PCR-96-FLT- |
| | | C |
| Optical 96-Well Reaction Plate | Bio-Rad | HSP-9601 |
| Optical 384-Well Reaction Plate | Bio-Rad | HSP-3805 |

### Apparatus

Water purification system (PURELAB Classic, ELGA, England)
Pipettors (Single/Multiple channels, Eppendorf, Germany)
CO₂ Incubator (Thermo, HERA CELL 240i, Germany)
QIAcube HT System (Qiagen, QIAcube HT, USA)
C1000 Touch Thermal Cycler (BIO-RAD, C1000 Touch, USA)
BIO-RAD CFX 384 Touch Real Time PCR System (BIO-RAD, CFX 384 Touch, USA)
NanoDrop ND-2000 Spectrophotometer (Thermo, NanoDrop ND2000, USA)
Centrifuge (Eppendorf, 5810R, Germany)
Microplate spectrophotometer (Molecular Devices, Spectra Max M2^{e}, USA)
Microplate Shaker (Thermo, 100-240V, 50-60 Hz, China)
Shaker (IKA, MS3 digital,
Germany)

### Preparation of Solution

### Preparation of Stock Solution:

| | | | Volume | |
|---|---|---|---|---|
| | | | of | |
| | Weight | | solvent | Concentration |
| Name | (mg) | solvent | (mL) | (mM) |
| Rifampicin | 9.99 | DMSO | 1.208 | 10 |

Note:
The stock solution was stored ≤ −30° C.

### Preparation of Dosing Solution

Fresh dosing solutions of test compounds, and Rifampicin were prepared in the incubation medium on the day of dosing. The detailed information of the dosing solution is listed in the following table.

| | | Concentration |
|---|---|---|
| Compound | | of Organic |
| Name | Final Concentration | Solvent |
| | 0.100, 1.00, 3.00, 10.0 | 0.1% DMSO |
| | and 30.0 μM | |
| Rifampicin | 10.0 μM | (v/v) |

### Hepatocyte Preparations

Cryopreserved hepatocytes were thawed and counted to determine yield, viability was measured. Hepatocytes at the concentrations of 0.7 million/mL were transferred to collagen-coated 96-well plates for attachment (0.1 mL viable cells/well). After allowing 4 to 5 hours for attachment of cells, the plating medium was replaced with Incubation Medium containing 2% (v/v) Matrigel™ (sandwich medium) and the hepatocytes were incubated until use.

### Hepatocyte Incubations

All incubations were conducted at 37° C., 5% CO₂, and saturated humidity.

The sandwich medium was removed and the hepatocytes were treated with incubation solutions containing test articles in vehicle (0.1% DMSO), or positive control for 24 hours after the cultures were established. The incubation solution was aspirated and replaced with incubation solution containing the same concentrations of test articles, vehicle, or positive controls for an additional 24 hours. The total treatment period was 48 hours. Triplicate were used for each concentration of test compound and positive control.

After treatment period of 48 hours, the incubation solution was aspirated. Hepatocytes were washed once with pre-warmed HBSS and incubated with 140 μL of RLT (lysis buffer from Rneasy 96 Kit) supplemented with 1% β-mercaptoethanol. Plates were then stored at ≤−60° C. freezer until RNA analysis.

### Cytotoxicity Assessment

Cytotoxic potential of test articles was evaluated by determining the activity of lactate dehydrogenase (LDH) in the incubation medium following incubations with test compounds and vehicle for 24 hours and 48 hours, respectively, using a commercially available LDH kit. Cell lysis solution was used as a positive control and incubation medium was used as a blank control in this assay.

### RNA Analysis

Before RNA extraction, the frozen plate was thawed and cell lysates were transferred to 96-well plates. RNA isolation was performed using QIAcube HT System. RNA concentration was measured with NanoDrop ND-2000 spectrophotometer. The purity of RNA preparation was calculated by the ratio of the OD260 nm/OD280 nm and the acceptable range is between 1.8 and 2.2. Reverse transcription was performed to obtain cDNA. Quantification of the selective genes by real time quantitative Polymerase Chain Reaction (qPCR) was performed with TaqMan universal PCR Master Mix on the BIO-RAD CFX 384 Touch Real Time PCR System. The following setting were used: 50° C. for 2 minutes, 95° C. for 10 minutes, 40 cycles of 95° C. for 15 seconds and 60° C. for 1 minute. 18S rRNA was used as the internal standard.

### Data Analysis

### Gene Expression

To account for variation in RNA yields and reverse transcription polymerase chain reaction (RT-PCR), the gene of interest in all samples was normalized to an internal control gene (18S ribosomal RNA) (Ct_{target gene}−Ct_{18S}=ΔCt).

The relative quantitation or change in mRNA level of selected genes induced by each test compound was expressed in relation to the vehicle control sample (Δctcompound−ΔCt_{vehicle}=ΔΔCt). Fold changes in gene expression were determined by taking 2 to the power of this value (2^{-ΔΔt}).

The percent of positive control was calculated with the formula below and reported.

% of positive control [(fold change in treated sample)−1]/[(fold change in positive control)−1]×100

### Classification and Criteria

| Parameters | Classification | Criteria |
|---|---|---|
| Gene expression for PC | Acceptable | >4 fold of |
| Gene expression | Non-inducer | vehicle control |
| for test compound | Inducer | <2 fold of |
| | Possible | vehicle control |
| | | and ≤ 20% of |
| | | PC |
| | | ≥2 fold of |
| | | vehicle control |
| | | and increased |
| | | in a |
| | | concentration- |
| | | dependent |
| | | manner |
| | | <2 fold of |
| | | vehicle control |
| | | and > 20% of |
| | | PC |

### Part II—Results

Experimental results are provided in Table 10, below. The symbol “+” indicates induction fold of less than or equal to 2-fold. The symbol “++” indicates induction fold of greater than 2-fold to less than or equal to 3-fold. The symbol “+++” indicates induction fold of greater than 3-fold to less than or equal to 10-fold. The symbol “++++” indicates induction fold of greater than 10-fold.

TABLE 10

| | CYP 3A4 | CYP 3A4 |
|---|---|---|
| | induction | induction |
| Compound | mRNA: Mean | mRNA: |
| No. | fold at 10 μM | Donor |
| I-39 | ++ | BXW |
| I-40 | ++ | BXW |
| I-40 | + | BXW |
| I-40 | +++ | VKB |
| I-40 | ++ | NFX |
| I-45 | ++ | BXW |
| I-51 | ++ | BXW |
| I-63 | +++ | BXW |
| I-63 | +++ | BXW |
| I-63 | +++ | NFX |
| I-63 | ++++ | VKB |
| I-71 | + | BXW |
| I-71 | + | BXW |
| I-71 | + | VKB |
| I-71 | + | NFX |
| I-84 | + | BXW |
| I-87 | + | BXW |
| I-88 | + | BXW |
| I-88 | + | BXW |
| I-88 | + | NFX |
| I-88 | + | VKB |
| I-114 | + | BXW |
| I-114 | + | BXW |
| I-114 | + | XSM |
| I-114 | + | NFX |
| I-118 | + | BXW |
| I-119 | + | BXW |
| I-121 | +++ | BXW |
| I-138 | + | BXW |
| I-142 | ++ | BXW |
| I-149 | + | BXW |
| I-149 | + | BXW |
| I-149 | +++ | XSM |
| I-149 | ++ | NFX |
| I-157 | + | BXW |
| I-173 | +++ | BXW |
| I-190 | +++ | BXW |
| I-220 | +++ | BXW |
| I-225 | +++ | BXW |
| I-256 | + | BXW |
| I-271 | + | BXW |
| I-281 | + | BXW |
| I-283 | ++++ | RSE |
| I-288 | ++++ | RSE |
| I-296 | + | BXW |
| I-296 | + | BX |
| I-296 | ++ | XSM |
| I-296 | + | NFX |
| I-314 | +++ | BXW |
| I-327 | + | BXW |
| I-327 | + | XSM |
| I-327 | + | NFX |
| I-359 | + | BXW |
| I-363 | + | BXW |
| I-363 | + | XSM |
| I-363 | + | NFX |
| I-370 | + | BXW |
| I-370 | + | XSM |
| I-370 | + | NFX |
| I-373 | +++ | RSE |
| I-394 | + | BXW |
| I-394 | + | XSM |
| I-394 | + | NFX |
| Compound A | +++ | RSE |
| Compound B | ++ | BXW |
| Compound C | +++ | BXW |

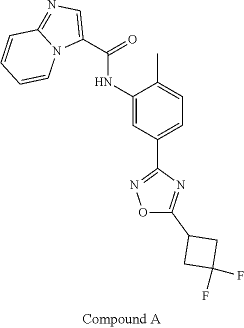 [SMILES: Cc1ccc(-c2noc(C3CC(F)(F)C3)n2)cc1NC(=O)c1cnc2ccccn12]

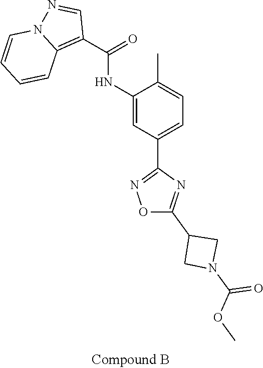 [SMILES: COC(=O)N1CC(c2nc(-c3ccc(C)c(NC(=O)c4cnn5ccccc45)c3)no2)C1]

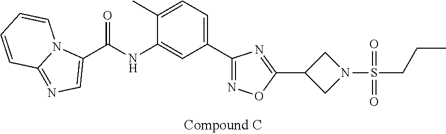 [SMILES: CCCS(=O)(=O)N1CC(c2nc(-c3ccc(C)c(NC(=O)c4cnc5ccccn45)c3)no2)C1]

### Example 102—Kinetic Solubility Assay Procedure

10 μL of 10 mM in DMSO of test article and control compounds was added into lower chambers of whatman miniuniprep vials, respectively. 490 μL of 50 mM PB (pH 7.4) was then added into lower chambers of the whatman miniuniprep vials, respectively. The solubility samples were then vortexed for at least 2 minutes. Miniuniprep vials were shaken for 24 hours at RT at the speed of 800 rpm, centrifuged 20 minutes (eg. 4000 rpm) and the filtrates were analyzed by UPLC-UV/HPLC-UV system to calculate the concentration with standard curve.

Filter Membrane: Miniuniprep (PTFE Filter Media with Polypropylene Housing) Cat. No. UN203NPUORG, GE Halthcare Whatman.

Experimental results are provided in Table 11, below. The symbol “****” indicates a solubility less than or equal to 2 μM. The symbol “***” indicates a solubility greater than 2 μM and less than or equal to 10 μM. The symbol “**” indicates a solubility greater than 10 μM and less than or equal to 50 μM. The symbol “*” indicates a solubility greater than 50 μM.

TABLE 11

| Compound No. | Solubility (μM) |
|---|---|
| I-1 | **** |
| I-2 | **** |
| I-3 | *** |
| I-4 | **** |
| I-5 | **** |
| I-6 | *** |
| I-7 | **** |
| I-8 | **** |
| I-9 | **** |
| I-10 | **** |
| I-11 | **** |
| I-12 | **** |
| I-13 | **** |
| I-14 | **** |
| I-15 | **** |
| I-16 | **** |
| I-17 | **** |
| I-18 | **** |
| I-19 | **** |
| I-20 | **** |
| I-21 | **** |
| I-22 | **** |
| I-23 | * |
| I-24 | **** |
| I-25 | **** |
| I-26 | **** |
| I-27 | **** |
| I-28 | **** |
| I-29 | **** |
| I-30 | **** |
| I-31 | **** |
| I-32 | **** |
| I-33 | *** |
| I-34 | **** |
| I-35 | **** |
| I-36 | **** |
| I-37 | **** |
| I-38 | **** |
| I-39 | **** |
| I-40 | **** |
| I-41 | **** |
| I-42 | **** |
| I-43 | **** |
| I-44 | **** |
| I-45 | **** |
| I-46 | **** |
| I-47 | **** |
| I-48 | **** |
| I-49 | **** |
| I-50 | *** |
| I-51 | **** |
| I-52 | **** |
| I-53 | **** |
| I-54 | **** |
| I-55 | **** |
| I-56 | **** |
| I-57 | **** |
| I-58 | ** |
| I-59 | * |
| I-60 | **** |
| I-61 | **** |
| I-62 | **** |
| I-63 | **** |
| I-64 | ** |
| I-65 | **** |
| I-66 | **** |
| I-67 | **** |
| I-68 | **** |
| I-69 | **** |
| I-70 | **** |
| I-71 | ** |
| I-72 | **** |
| I-73 | **** |
| I-74 | **** |
| I-75 | **** |
| I-76 | **** |
| I-77 | **** |
| I-78 | **** |
| I-79 | **** |
| I-80 | * |
| I-81 | ** |
| I-82 | **** |
| I-83 | **** |
| I-84 | ** |
| I-85 | **** |
| I-86 | **** |
| I-87 | **** |
| I-88 | **** |
| I-90 | **** |
| I-91 | **** |
| I-92 | **** |
| I-93 | **** |
| I-94 | **** |
| I-95 | **** |
| I-96 | **** |
| I-97 | ** |
| I-98 | **** |
| I-99 | * |
| I-100 | * |
| I-434 | **** |
| I-435 | **** |
| I-101 | **** |
| I-102 | **** |
| I-103 | **** |
| I-104 | **** |
| I-105 | **** |
| I-106 | * |
| I-107 | **** |
| I-108 | **** |
| I-109 | **** |
| I-110 | * |
| I-111 | * |
| I-112 | ** |
| I-113 | **** |
| I-436 | **** |
| I-400 | **** |
| I-437 | **** |
| I-438 | **** |
| I-122 | **** |
| I-114 | **** |
| I-115 | **** |
| I-116 | * |
| I-117 | * |
| I-118 | * |
| I-119 | **** |
| I-121 | **** |
| I-120 | **** |
| I-123 | **** |
| I-124 | **** |
| I-125 | **** |
| I-126 | **** |
| I-128 | **** |
| I-129 | **** |
| I-130 | **** |
| I-131 | **** |
| I-132 | **** |
| I-133 | **** |
| I-134 | **** |
| I-135 | **** |
| I-136 | **** |
| I-137 | **** |
| I-138 | **** |
| I-139 | * |
| I-140 | *** |
| I-141 | ** |
| I-143 | **** |
| I-144 | ** |
| I-145 | ** |
| I-146 | * |
| I-147 | ** |
| I-148 | ** |
| I-149 | **** |
| I-150 | **** |
| I-154 | **** |
| I-155 | ** |
| I-157 | * |
| I-159 | * |
| I-160 | **** |
| I-165 | **** |
| I-166 | **** |
| I-161 | **** |
| I-162 | * |
| I-163 | **** |
| I-164 | **** |
| I-167 | *** |
| I-168 | ** |
| I-170 | ** |
| I-171 | * |
| I-172 | **** |
| I-173 | **** |
| I-174 | *** |
| I-175 | **** |
| I-176 | ** |
| I-177 | **** |
| I-178 | **** |
| I-179 | * |
| I-180 | * |
| I-181 | * |
| I-182 | ** |
| I-183 | **** |
| I-184 | **** |
| I-185 | **** |
| I-186 | *** |
| I-187 | ** |
| I-188 | *** |
| I-189 | ** |
| I-190 | *** |
| I-191 | **** |
| I-192 | **** |
| I-193 | **** |
| I-194 | *** |
| I-195 | *** |
| I-196 | ** |
| I-197 | *** |
| I-198 | *** |
| I-199 | **** |
| I-200 | *** |
| I-201 | *** |
| I-202 | *** |
| I-203 | **** |
| I-204 | *** |
| I-205 | **** |
| I-206 | **** |
| I-207 | **** |
| I-208 | **** |
| I-209 | **** |
| I-210 | ** |
| I-211 | *** |
| I-212 | *** |
| I-213 | **** |
| I-214 | *** |
| I-216 | ** |
| I-217 | ** |
| I-218 | **** |
| I-219 | *** |
| I-220 | **** |
| I-221 | **** |
| I-224 | * |
| I-225 | ** |
| I-226 | * |
| I-227 | ** |
| I-228 | ** |
| I-229 | **** |
| I-230 | ** |
| I-231 | ** |
| I-232 | ** |
| I-233 | ** |
| I-234 | ** |
| I-235 | *** |
| I-236 | *** |
| I-237 | ** |
| I-238 | * |
| I-239 | ** |
| I-240 | ** |
| I-241 | **** |
| I-242 | *** |
| I-243 | * |
| I-244 | * |
| I-245 | **** |
| I-246 | **** |
| I-247 | *** |
| I-248 | *** |
| I-249 | *** |
| I-250 | * |
| I-251 | **** |
| I-252 | *** |
| I-253 | ** |
| I-254 | *** |
| I-255 | ** |
| I-256 | * |
| I-257 | * |
| I-445 | **** |
| I-258 | *** |
| I-259 | ** |
| I-260 | *** |
| I-261 | ** |
| I-262 | ** |
| I-263 | * |
| I-264 | ** |
| I-265 | ** |
| I-266 | **** |
| I-268 | * |
| I-269 | *** |
| I-270 | * |
| I-271 | * |
| I-272 | **** |
| I-273 | *** |
| I-274 | **** |
| I-275 | *** |
| I-276 | ** |
| I-277 | ** |
| I-278 | **** |
| I-279 | ** |
| I-280 | **** |
| I-281 | *** |
| I-282 | ** |
| I-283 | ** |
| I-284 | *** |
| I-285 | **** |
| I-286 | **** |
| I-287 | *** |
| I-288 | **** |
| I-289 | *** |
| I-290 | *** |
| I-291 | *** |
| I-292 | *** |
| I-293 | *** |
| I-294 | ** |
| I-295 | **** |
| I-296 | ** |
| I-297 | **** |
| I-298 | ** |
| I-299 | **** |
| I-300 | **** |
| I-301 | **** |
| I-302 | **** |
| I-303 | **** |
| I-304 | **** |
| I-305 | **** |
| I-306 | * |
| I-307 | ** |
| I-308 | *** |
| I-309 | *** |
| I-310 | **** |
| I-311 | **** |
| I-312 | **** |
| I-313 | *** |
| I-314 | *** |
| I-315 | **** |
| I-316 | **** |
| I-317 | ** |
| I-318 | *** |
| I-319 | *** |
| I-320 | *** |
| I-321 | **** |
| I-322 | **** |
| I-323 | **** |
| I-324 | **** |
| I-325 | **** |
| I-326 | * |
| I-327 | * |
| I-328 | *** |
| I-329 | **** |
| I-330 | ** |
| I-331 | ** |
| I-332 | **** |
| I-333 | **** |
| I-334 | **** |
| I-335 | * |
| I-336 | * |
| I-337 | * |
| I-338 | **** |
| I-339 | **** |
| I-340 | **** |
| I-341 | **** |
| I-342 | *** |
| I-343 | ** |
| I-344 | *** |
| I-345 | **** |
| I-346 | *** |
| I-347 | **** |
| I-348 | *** |
| I-349 | **** |
| I-350 | ** |
| I-351 | **** |
| I-352 | **** |
| I-353 | **** |
| I-354 | **** |
| I-355 | **** |
| I-356 | ** |
| I-357 | *** |
| I-358 | ** |
| I-359 | * |
| I-360 | ** |
| I-361 | **** |
| I-362 | *** |
| I-363 | ** |
| I-364 | * |
| I-365 | *** |
| I-366 | **** |
| I-367 | **** |
| I-368 | **** |
| I-369 | ** |
| I-370 | * |
| I-371 | * |
| I-372 | ** |
| I-373 | *** |
| I-374 | **** |
| I-375 | **** |
| I-376 | **** |
| I-377 | *** |
| I-378 | **** |
| I-379 | ** |
| I-380 | **** |
| I-381 | **** |
| I-382 | **** |
| I-383 | **** |
| I-384 | **** |
| I-385 | **** |
| I-386 | **** |
| I-387 | *** |
| I-388 | *** |
| I-389 | ** |
| I-390 | *** |
| I-391 | * |
| I-392 | *** |
| I-393 | *** |
| I-394 | * |
| I-395 | ** |
| I-396 | **** |
| I-397 | *** |
| I-398 | *** |
| I-399 | *** |
| Compound A | **** |
| I-446 | ** |

### Example 103—BCRP Inhibition: Vesicular Transport Assays

Vesicular transport assays are performed with inside-out membrane vesicles prepared from cells overexpressing human ABC transporters. The transporters are expressed by SOLVO Biotechnology mammalian (HEK293) cells. The mammalian cells are stably transfected with the ABC transporter BCRP. Test articles are incubated with membrane vesicle preparations and the probe substrate (E3S, 1 μM). Incubations are conducted in the presence of 4 mM ATP or AMP to distinguish between transporter-mediated uptake and passive diffusion into the vesicles. Test compounds are added to the reaction mixture in 0.75 μL of solvent (1% of the final incubation volume). Reaction mixtures are preincubated for 15 minutes at 32±1° C. Reactions are initiated by the addition of 25 μL of pre-warmed 12 mM MgATP (or 12 mM AMP in assay buffer as a background control). Reactions are quenched by the addition of 200 μL of ice-cold washing buffer and immediate filtration via glass fiber filters mounted to a 96-well plate (filter plate). The filters are washed (5×200 μL of ice-cold washing buffer), dried and the amount of substrate inside the filtered vesicles is determined by liquid scintillation counting.

### Example 104—NanoBRET Cellular Assay for wtKIT and CSF-1R

Exemplary compounds were tested for their ability to inhibit target engagement of KIT or CSF-1R kinases through competitive displacement of a Nano-Luc luciferase kinase fusion in HEK293 cells.

The NanoBRET target engagement assay employs an energy transfer technique designed to measure molecular proximity in living cells. The assay measures the apparent affinity of test compounds by competitive displacement of the NanoBRET tracer, reversibly bound to a NanoLuc luciferase-kinase fusion construct in cells. The intracellular binding affinity and selectivity are physiologically relevant and fundamental to the pharmacological mechanism of the compounds. While biochemical and biophysical assays identify the kinase inhibitors in vitro, the NanoBRET target engagement assay serves to determine the direct interaction of the compounds binding to target kinases in cells.

### Part 1—Procedures

HEK293 cells transiently expressing NanoLuc-DDR1 fusion vector were seeded into 384-well plates and treated with the Tracer K-4 and compound for 1 hour. The BRET signal was measured on an EnVision 2104 multilabel microplate reader. NanoBRET Target Engagement Assay in HEK293 cells transiently transfected with KIT and CSF1R-NanoLuc Fusion Vector. HEK293 cells were transfected with 1 pg KIT or CSF1R-NanoLuc Fusion Vector and 9 pg carrier DNA. The transfected cells were treated with test compounds (starting at 10 μM, 10-dose with 3-fold dilution) and reference compound (desatinib) (starting at 1 μM, 10-dose with 3-fold dilution). Percent inhibition due to compound activity was calculated to determine IC₅₀.

### Part II—Results

Experimental results are provided in Table 12, below. The symbol “****” indicates a IC₅₀ less than or equal to 0.1 μM. The symbol “***” indicates a IC₅₀ greater than 0.1 μM and less than or equal to 1.0 μM. The symbol “**” indicates a IC₅₀ greater than 1.0 μM and less than or equal to 10 μM. The symbol “*” indicates a IC₅₀ greater than 10 μM.

TABLE 12

| Compound | KIT IC₅₀ | CSF-1R IC₅₀ |
|---|---|---|
| No. | (μM) | (μM) |
| I-71 | **** | * |
| I-88 | **** | * |
| I-110 | **** | |
| I-114 | **** | * |
| I-296 | **** | |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

LENGTHY TABLES
The patent application contains a lengthy table section. A copy of the table is available in electronic form from the USPTO web site (https://seqdata.uspto.gov/?pageRequest=docDetail&DocID=US20240254119A1). An electronic copy of the table will also be available from the USPTO upon request and payment of the fee set forth in 37 CFR 1.19(b)(3).

1. A compound represented by Formula I: 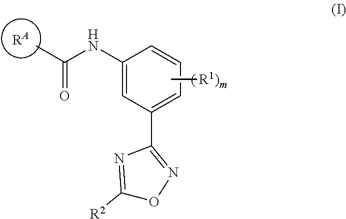
or a pharmaceutically acceptable salt thereof; wherein:
R¹ represents independently for each occurrence, —CN, C₁₋₆ alkyl, or C₁₋₆ haloalkyl;
R² is a 3 membered saturated monocyclic carbocyclic ring wherein R² is substituted with p occurrences of R⁶
R⁶ represents independently for each occurrence oxo, halogen, C₁₋₆ aliphatic, C₁₋₆ haloaliphatic, —CN, —NO₂, —OR, —OCR₃, —SR, —NR₂, —S(O)₂R, —S(O)₂NR₂, —S(O)R, —S(O)NR₂, —C(R)₂OR, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, or optionally substituted phenyl;
R^{A} is of any of the following structures: 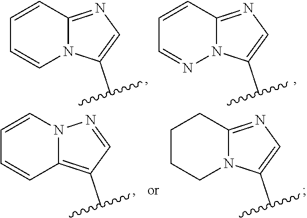
each of which is substituted by n occurrences of R³;
R³ represents independently for each occurrence oxo, —CN, —NO₂, —OR, —OCR₃, —SR, —NR₂, —S(O)₂R, —S(O)₂NR₂, —S(O)R, —S(O)NR₂, —C(R)₂OR, —C(R)₂OCR₃—C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, -L²-R⁵, or an optionally substituted group selected from C₁₋₆ haloaliphatic, phenyl, naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-11 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-3 heteroatoms independently selected from nitrogen, oxygen, and sulfur; each of which is substituted with r instances of R; or:
two R³ groups on adjacent carbon atoms are taken together with the carbon atoms to which they attach to form an optionally substituted 4-7 membered saturated monocyclic ring having 0-3 heteroatoms independently selected from nitrogen, oxygen, and sulfur, substituted with r instances of R;
L² represents independently for each occurrence a C₁₋₆ bivalent saturated or unsaturated, straight or branched hydrocarbon chain wherein one, two, or three methylene units of the chain are independently replaced by —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, —S(O)₂— or -Cy-;
Cy represents independently for each occurrence phenyl, a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
R⁵ represents independently for each occurrence hydrogen, OR, C₁₋₆ aliphatic, C₁₋₆ haloaliphatic, or phenyl fused to a 5-6 membered saturated heterocyclic ring having 1-2 heteroatoms independently selected from nitrogen, oxygen, and sulfur;
each R is independently hydrogen, —CN, halogen, oxo, or an optionally substituted group selected from C₁₋₆ aliphatic; C₁₋₆ haloaliphatic; C₁₋₃ hydroxyalkyl; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur;
m is 0 or 1;
n is 1, 2, 3, 4, or 5;
p is 0, 1, 2, 3, 4, or 5; and
r is 0, 1, 2, 3, 4, or 5.

2-3. (canceled)

4. The compound of claim 1, wherein at least one R is C₁₋₆ alkyl.

5-8. (canceled)

9. The compound of claim 1, wherein the compound is represented by one of the following or a pharmaceutically acceptable salt thereof:

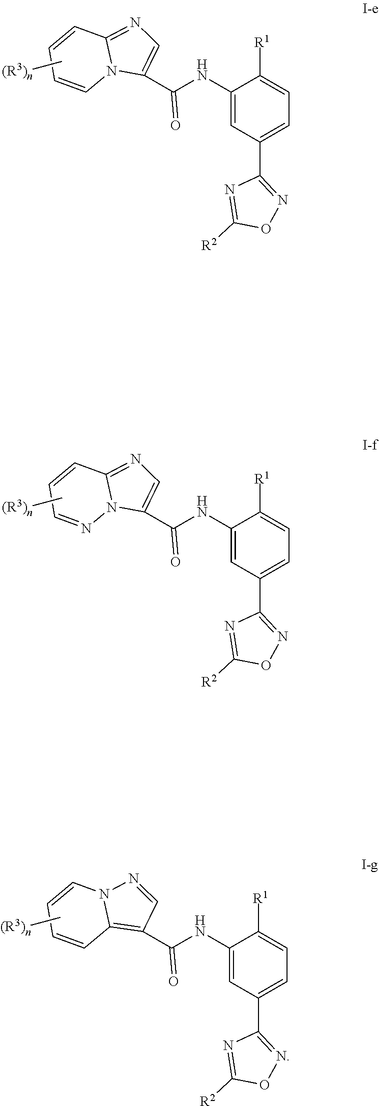

10. The compound of claim 1, wherein the compound is represented by the following or a pharmaceutically acceptable salt thereof:

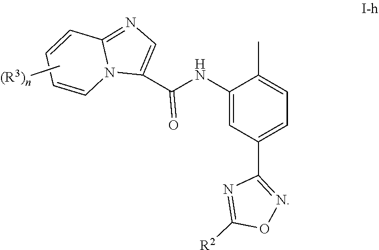

11-16. (canceled)

17. The compound of claim 1, wherein the compound is represented by one of the following or a pharmaceutically acceptable salt thereof:

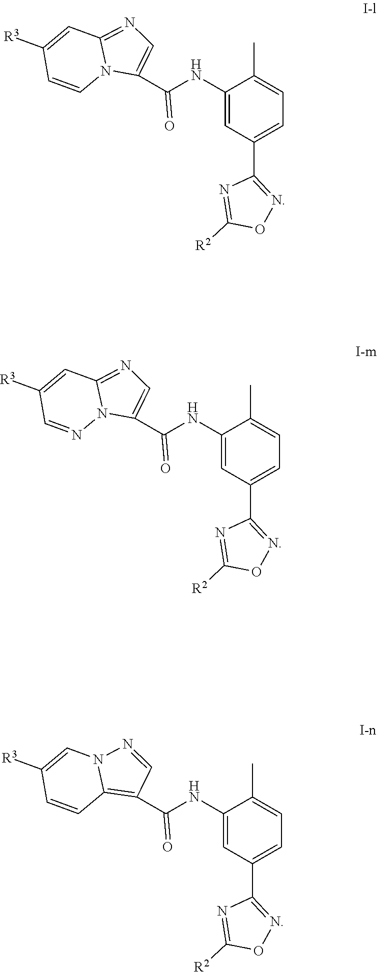

18-44. (canceled)

45. The compound of claim 9, wherein each R³ is independently selected from:

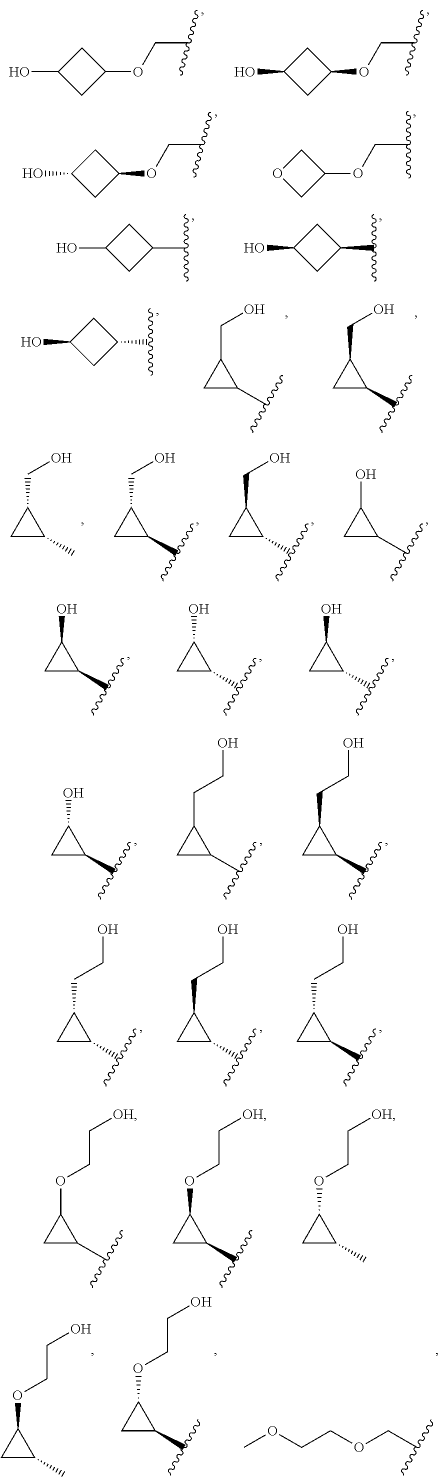

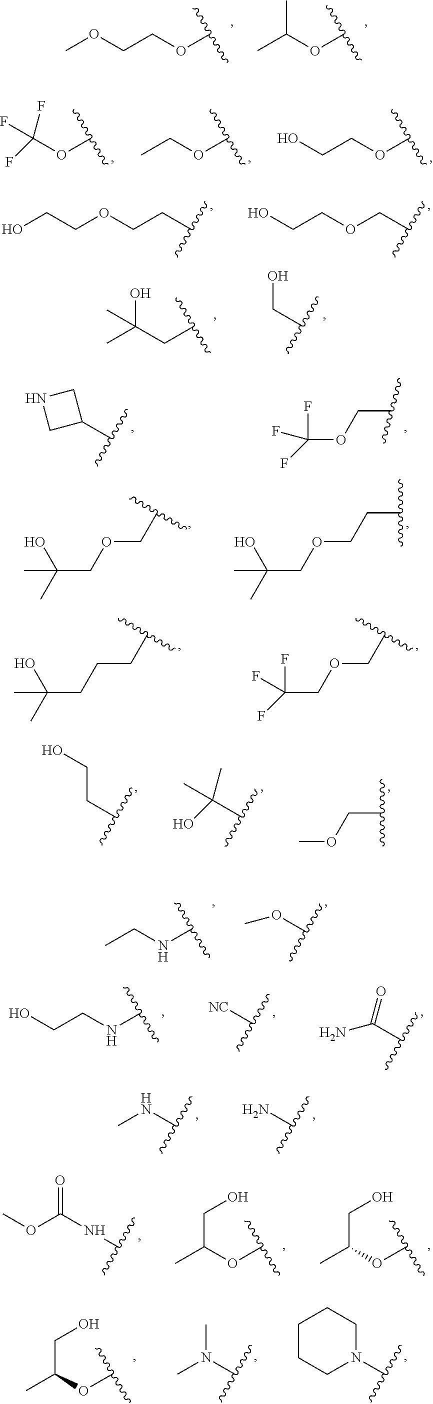

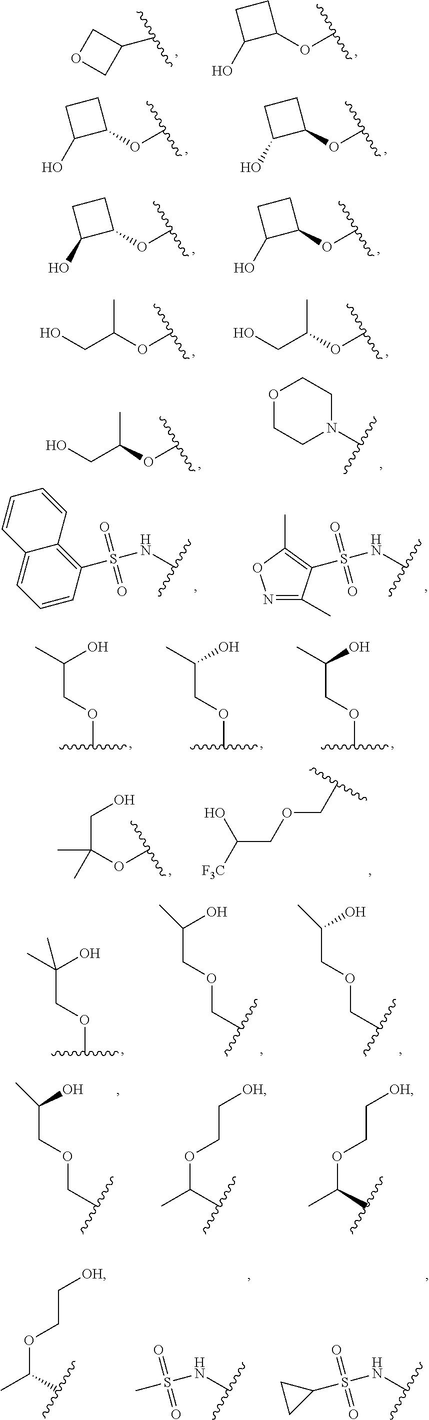

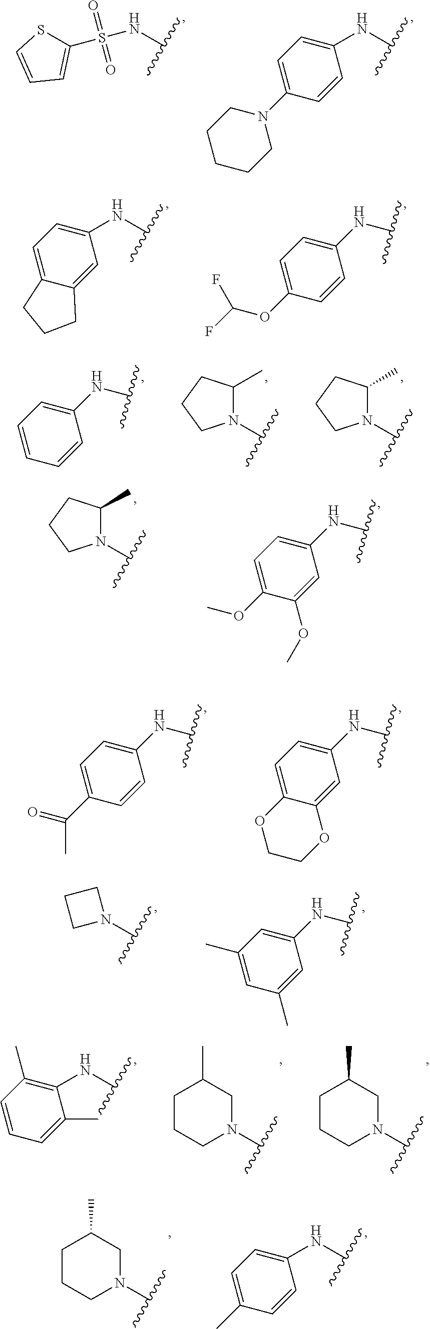

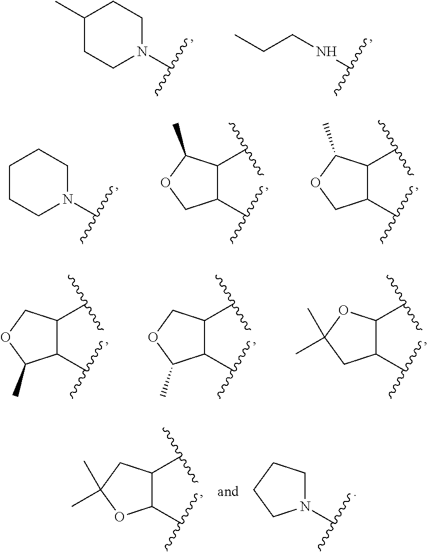

46. The compound of claim 10, wherein each R³ is independently selected from:

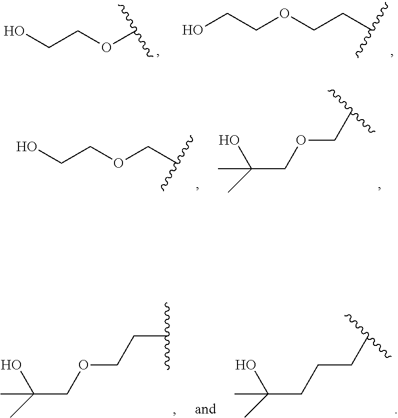

47. (canceled)

48. The compound of claim 10, wherein each R³ is independently selected from:

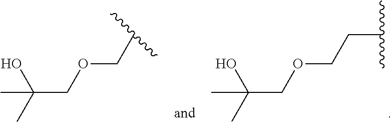

49-60. (canceled)

61. The compound of claim 9, wherein R² is independently selected from:

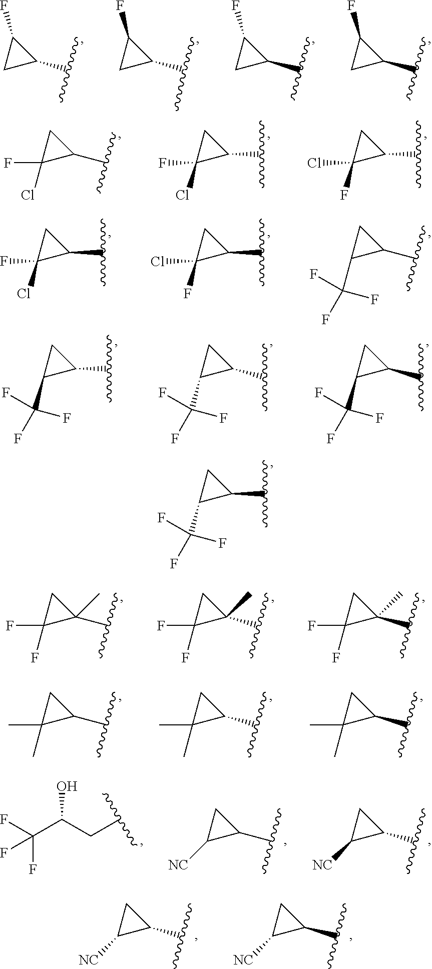

62. The compound of claim 61, wherein R² is selected from:

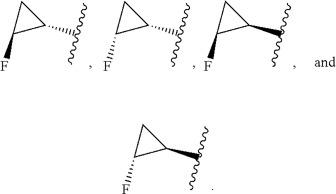

63. The compound of claim 62, wherein R² is

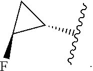 [SMILES: *[C@H]1C[C@@H]1F]

64. (canceled)

65. The compound of claim 1, wherein the compound is represented by one of the following or a pharmaceutically acceptable salt thereof:

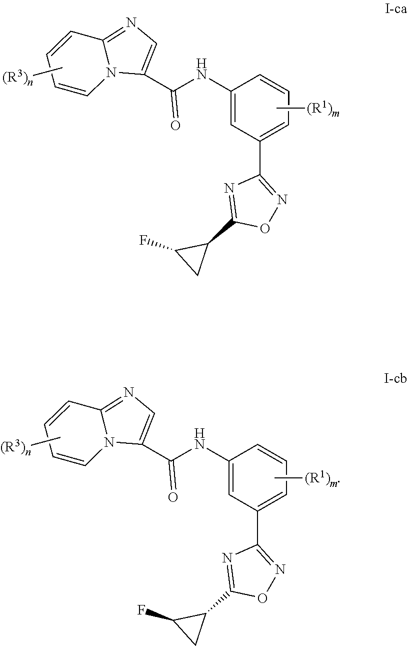

66. The compound of claim 1, wherein the compound is represented by one of the following or a pharmaceutically acceptable salt thereof:

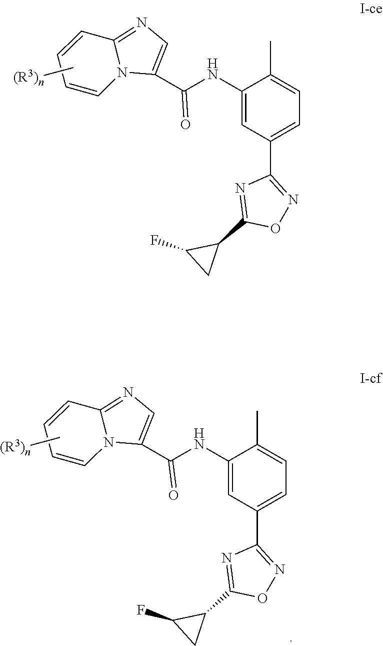

67. The compound of claim 66, wherein n is 1 or 2, wherein at least one R³ is selected from:

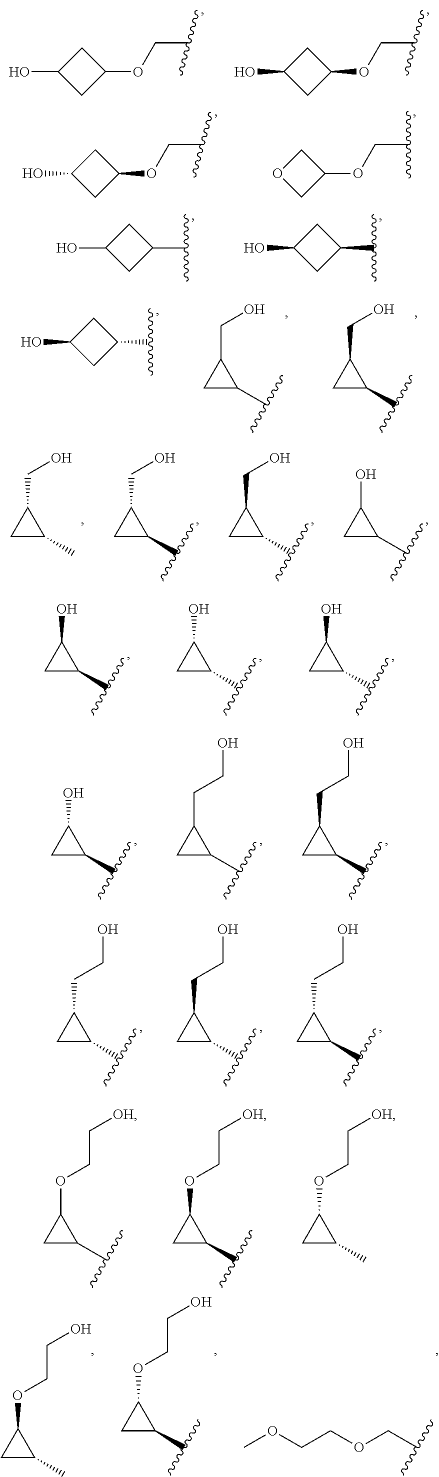

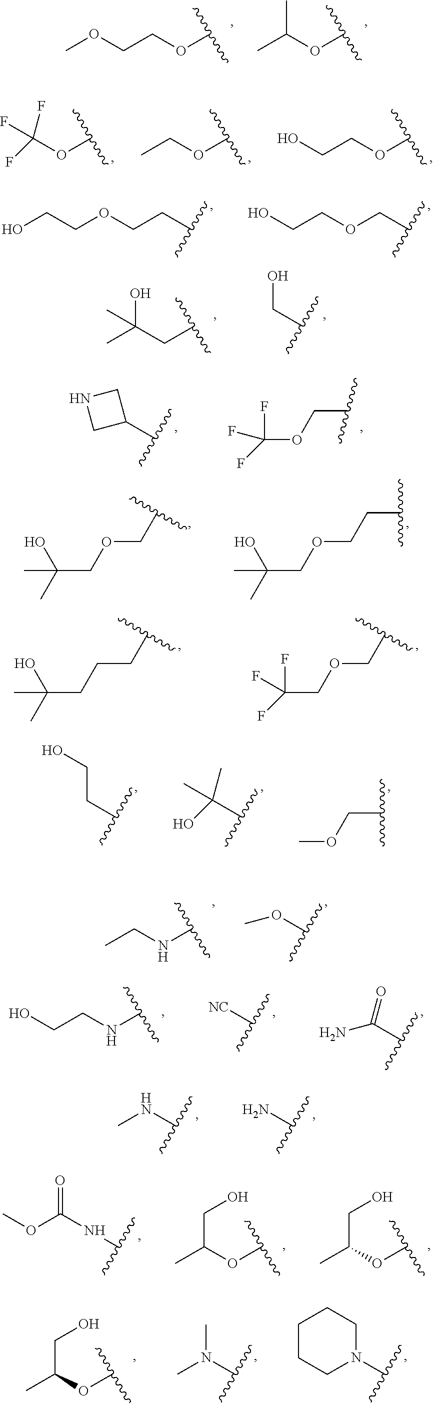

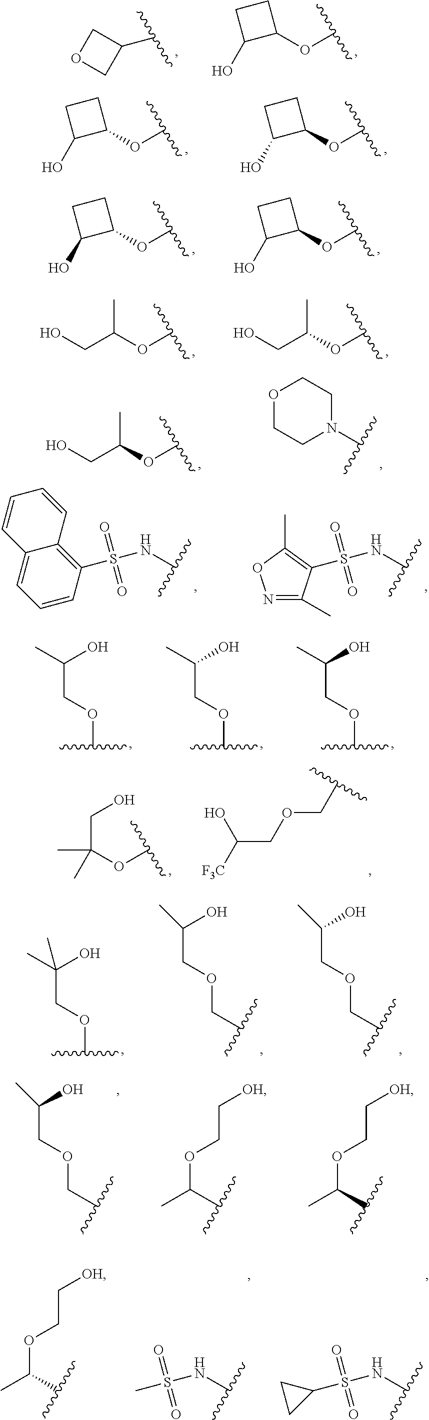

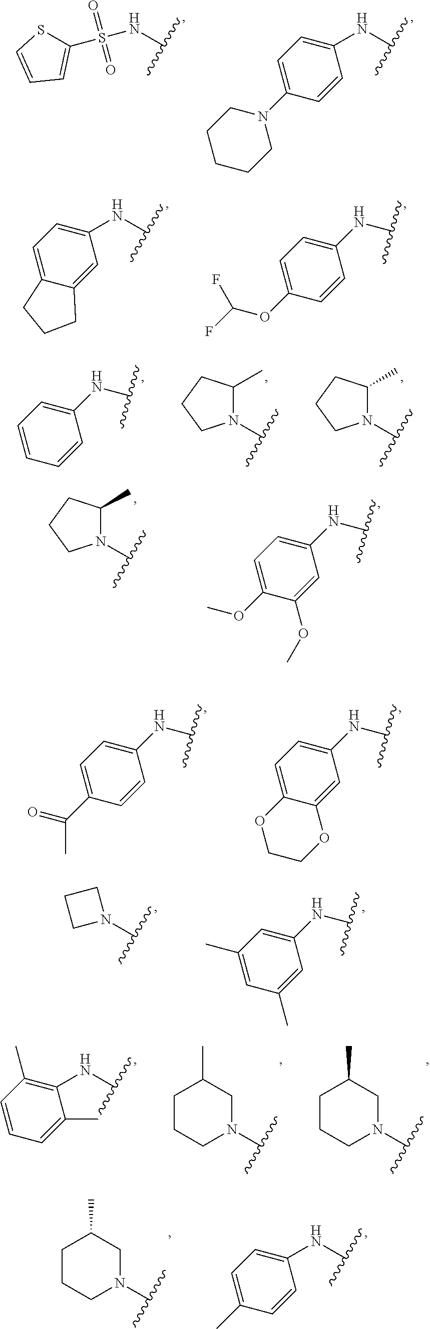

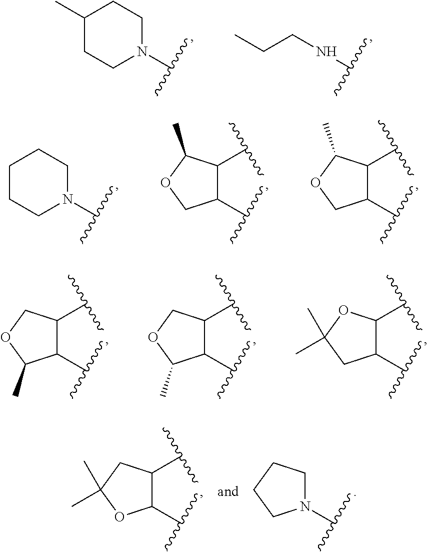

68. The compound of claim 67, wherein each R³ is independently selected from:

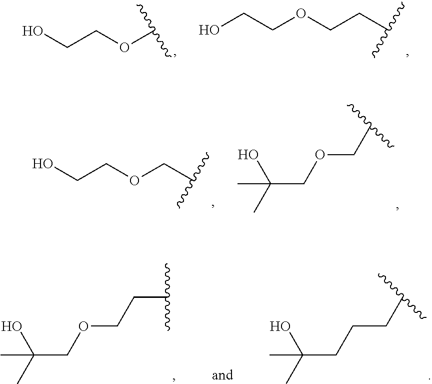

69. The compound of claim 68, wherein n is 1.

70-74. (canceled)

75. A compound, selected from those depicted in Table 1 herein, or a pharmaceutically acceptable salt thereof.

76. A pharmaceutical composition comprising a compound of claim 1 and a pharmaceutically acceptable carrier.

77. A method of inhibiting the activity of a c-kit kinase in a patient, comprising administering to said patient a compound of claim 1.

78. A method of treating a c-kit kinase mediated disease or disorder in a patient, comprising administering to said patient a compound of claim 1.

79. The method according to claim 78, wherein the c-kit kinase mediated disease or disorder is a mast-cell associated disease, a respiratory disease, an inflammatory disorder, an autoimmune disorder, a metabolic disease, a fibrotic disease, a dermatological disease, an allergic disease, a cardiovascular disease, or a neurological disorder.

80. The method according to claim 78, wherein the c-kit kinase mediated disease or disorder is asthma, allergic rhinitis, pulmonary arterial hypertension (PAH), primary pulmonary hypertension (PPH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, food allergy, chronic rhinosinusitis, type I diabetes, type II diabetes, systemic sclerosis, allergic keratoconjunctivitis, vernal keratoconjunctivitis, Crohn's disease, or systemic and cutaneous lupus erythematosus and dermatomyositis.

81. The method according to claim 78, wherein the c-kit kinase mediated disease or disorder is mast cell gastrointestinal disease, prurigo nodularis, allergic conjunctivitis, eosinophilic esophagitis, mast cell activation syndrome, eosinophilic gastritis and/or eosinophilic duodenitis (EG/EoD), ulcerative colitis, eosinophilic gastritis (EG), or eosinophilic colitis (EC).

82. The method of claim 78, wherein the disease or disorder is urticaria.

83. The method of claim 78, wherein the patient is a human.

84. The compound of claim 1, selected from:

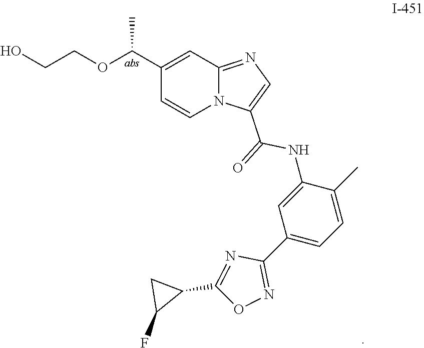 [SMILES: Cc1ccc(-c2noc([C@H]3C[C@@H]3F)n2)cc1NC(=O)c1cnc2cc([C@@H](C)OCCO)ccn12]

85. The compound of claim 1, selected from:

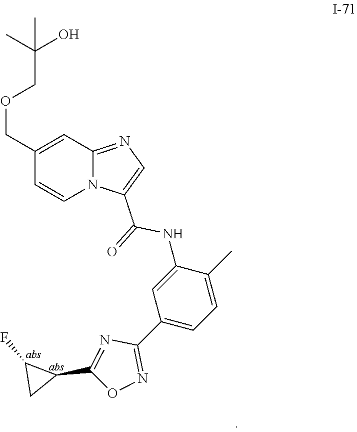 [SMILES: Cc1ccc(-c2noc([C@H]3C[C@@H]3F)n2)cc1NC(=O)c1cnc2cc(COCC(C)(C)O)ccn12]

86. The compound of claim 1, selected from:

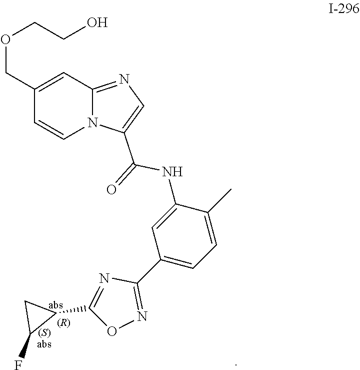

87. The compound of claim 1, selected from:

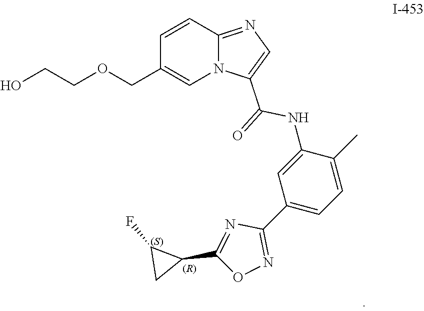

88. The compound of claim 1, selected from:

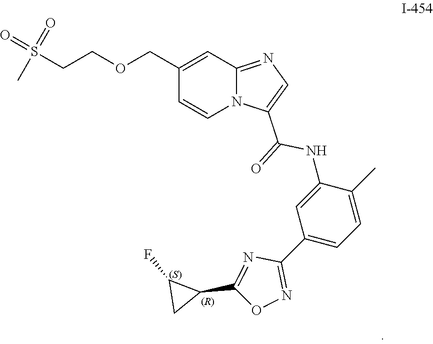

89. The pharmaceutical composition of claim 84, comprising a pharmaceutically acceptable carrier.

90. The pharmaceutical composition of claim 85, comprising a pharmaceutically acceptable carrier.

91. The pharmaceutical composition of claim 86, comprising a pharmaceutically acceptable carrier.

92. The pharmaceutical composition of claim 87, comprising a pharmaceutically acceptable carrier.

93. The pharmaceutical composition of claim 88, comprising a pharmaceutically acceptable carrier.
